(19) United States
(12) Patent Application Publication
(10) Pub. No.: US 2008/0254459 A1
(43) Pub. Date: Oct. 16, 2008

(54) Methods and Biomarkers for Detecting Nanoparticle Exposure

(75) Inventor: Mary Jane Cunningham, The Woodlands, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: (HARC) HOUSTON ADVANCED RESEARCH CENTER, The Woodlands, TX (US)

(21) Appl. No.: 11/817,661

(22) PCT Filed: Mar. 6, 2006
(86) PCT No.: PCT/US06/07942

§ 371 Date: Aug. 31, 2007

Related U.S. Application Data
(60) Provisional application No. 60/658,881, filed on Mar. 5, 2005.

(51) Int. Cl. C12Q 1/68

(52) U.S. Cl. 435/6

(57) ABSTRACT

Methods for gene expression profiling for exposure to nanoscale particulates or nanomaterials is provided together with identified biomarkers for nanomaterial exposure. A toxicogenomic exposure profile for nanomaterial contact is provided in accordance with a systems biology approach by iteratively sampling a test system several times after contact with nanomaterials of various chemical types.

### CROSS REFERENCE TO RELATED APPLICATIONS

This application claims priority to U.S. Provisional Application Ser. No. 60/658,881, filed Mar. 5, 2005, which is incorporated herein by reference in its entirety.

### STATEMENT REGARDING GOVERNMENT INTERESTS

This work was supported in part by the following United States Government grants: SGER Award No. BES-0436366 from the National Science Foundation. The Government may have certain rights in this invention.

### FIELD OF THE INVENTION

This invention relates generally to biomarkers for detection of nanoparticle exposure. The present invention relates more particularly to nanoparticle toxicity assessment using gene expression array profiling.

### BACKGROUND OF THE INVENTION

Without limiting the scope of the invention, its background is described in connection with gene expression profiling of cells exposed to nanomaterials and the identification of biomarkers for nanomaterial exposure. Nanomaterials are being developed and manufactured on a commercial scale. However, preliminary reports, referring primarily to carbon nanotubes, are mixed as to their toxicity.

Two studies done at Warsaw University by Huczko et al. showed no inflammation or toxicity when SWNT (single-walled carbon nanotubes) were instilled into the lungs of guinea pigs, or with dermal or optic contact in humans and rabbits, respectively. Huczko A, et al. Fullerene Sci. Technol. 9(2) (2001) 251-254; Huczko A and H Lange. Fullerene Sci. Technol. 9(2) (2001) 247-250. In addition, Pantarotto et al. (J Chem. Commun. (Cambridge) (1) (2004) 16-17) did not observe any toxicity at 1-10 micromolar levels in human 3T6 cells in culture. Warheit et al. (Toxicol. Sci. 77(1) (2004): 117-125) conducted an inhalation study with rats and found similar histopathological findings (the presence of granulomas) but interpreted these findings as “inconclusive” and may be “artifactual.” Even though 15% mortality was observed in the rats, it was concluded that the SWNT agglomerates led to the physical occlusion of the animals' airways causing suffocation and mortality was not due to toxicity of the SWNT themselves. Recently, a study by Maynard et al. (J. Toxicol. Environ. Health A 67(1) (2004) 87-104) evaluated nanotube deposition during their manufacture and handling and concluded that the risk of adverse effects from exposure is low.

However, in other studies, SWNT were found to be cytotoxic and produce oxidative stress in an immortalized human embryonic kidney (HEK) cell line. See Shvedova A A et al. J. Toxicol. Environ. Health A 66(20) (2003) 1909-1926. Lam et al. (Toxicol. Sci. 77(1) (2004) 126-134) exposed mice to SWNT and interpreted the results as the nanotubes being more toxic than quartz dust, which is already known to be a causative factor in silicosis. In addition, three recent publications reported toxicity of quantum dots and fullerene molecules. Sayes et al. (Nano Letters 4(10) (2004) 1881-1887) and Derfus et al. (Nano Letters 4(1) (2004) 11-18) showed cytotoxicity in human dermal fibroblasts and rat hepatocytes respectively. All of these reports assessed the toxicity of SWNT by traditional toxicity assays such as dermal absorption and inhalation (e.g. mice, rats, guinea pigs, rabbits).

Toxicogenomics is a term that has recently been applied to the study of toxicity using genomics, proteomics, metabolomics and other “OMIC” technologies. These technologies include: genotyping for adverse effects by investigating the incidence of SNPs in a species, gene expression profiling using gene expression microarray (GEM) and protein expression profiling using either protein arrays or two-dimensional gel electrophoresis and mass spectroscopy.

Gene expression profiling has been widely applied to monitor gene expression of various perturbations of cells and tissues using GEM. In the pharmaceutical arena, GEM analysis is now being used as a screening tool for thousands of drug candidates. By gene expression profiles, it is possible to characterize profiles which match known toxic compounds and thereby screen out unsuccessful candidates and reduce the number of failures further in the development pipeline.

OMIC technologies, including using GEM profiling, are now being applied to environmental toxicology. See, e.g. Cunningham M. J. et al. Annals of the New York Academy of Sciences 919 (2000) 52-67; U.S. Pat. No. 6,403,778 “Toxicological response markers”, Incyte Genomics; U.S. Pat. No. 6,372,431 “Mammalian toxicological response markers”, Incyte Genomics.

However, systems for broad toxicity assessment by gene expression profiling of nanomaterials are not available. Methods are needed for identifying nanomaterial or nanoparticle exposure, both generally and specifically by gene expression profiling. Biomarkers for nanomaterial exposure are further needed that can be used to monitor research and development, quality assurance and manufacturing processes of nanomaterials as well as environmental exposure of humans and other species to these materials.

### BRIEF SUMMARY OF THE INVENTION

The present invention is directed to a method of gene expression profiling for detecting exposure to nanoscale particulates or nanomaterials. Biomarkers have been identified that indicate such exposure. In one embodiment, a toxicogenomic exposure profile for nanomaterial contact is developed in accordance with a comprehensive systems biology approach by iteratively sampling a test system several times after contact with nanomaterials of various chemical types.

In one embodiment, methods and systems are provided for monitoring the fate of disposal and dispersal of nanomaterials in the environment.

In another embodiment, gene expression profiles of cell exposure to nanoscale materials are provided.

In another embodiment, biomarkers are provided for monitoring nanoparticle exposure in humans and other species as well as in-field monitoring of both internal and external environments. One embodiment provides diagnostic kits for such monitoring.

In one embodiment, a method is provided for detecting exposure of a cell to a nanomaterial comprising: a) generating a cDNA or cRNA population from a cell that has been in contact with, or is suspected of having been in contact with, a nanomaterial; b) contacting the cDNA or cRNA under hybridization conditions with a microarray comprising a plurality of polynucleotide sequences that each represent genes or gene specific portions of genes, said microarray including one or more biomarker genes or gene specific portion of the biomarker genes that are up or down regulated by exposure to the nanomaterial; and c) determining a relative degree of hybridization with the polynucleotide sequences comprising the microarray, as compared with a control sample; wherein an increase or decrease relative degree of hybridization with the biomarker gene polynucleotide sequence indicates contact of the cell with the nanomaterial.

By the phrase “genes or gene specific portions of gene” it is meant, in accordance with the understanding of those of skill in the art, that microarrays typically utilize gene specific oligonucleotide sequences of less than approximately 100 nucleotides and not full coding regions. Those of skill in the art are able to readily generate gene specific portions of the biomarker genes identified by the present inventors, such as by comparison with other known genes using sequence comparision software and search engines such as the NCBI BLASTn resource.

In one embodiment of the method, the nanomaterial is selected from the group consisting of FC, SiO₂, CB, TiO₂, and CNT. In another embodiment, the microarray includes polynucleotide sequences that each represent genes or gene specific portions of biomarker genes or gene families selected from the group set out on FIGS. 9A-C, and combinations thereof. In one embodiment of the invention, biomarker genes Kallikrein 5, Nice-1, and combinations thereof are provided as indicative of nanomaterial exposure, either alone or together with one or members of the group set out on FIGS. 9A-C, and combinations thereof.

In another embodiment of the invention, the microarray includes polynucleotide sequences that each represent genes or gene specific portions of SWNT biomarker genes selected from the group consisting of: DNA-damage-inducible transcript 3 (DDIT3); serum/glucocorticoid regulated kinase (SGK); N-myc downstream regulated gene 1 (NDRG1); AXIN1 up-regulated (AXUD1); and combinations thereof.

In another embodiment, biomarker genes for nanoparticle exposure are provided including Kallikrein 5 and/or Nice-1 in addition to one or more of Cystic fibrosis antigen Clone 24421; Hypothetical protein LOC221810; (LGALS7); S100 calcium binding protein A8 (S100A8); Uridine phosphorylase (UP); Bone morphogenetic protein receptor type IA (BMPR1A); Neurexin 2 (NRXN2); Rh type C glycoprotein (RHCG); Stromal cell-derived factor 2-like 1 (SDF2L1); Hypothetical protein SMAP31 (SMAP31); DNA-damage-inducible transcript 3 (DDIT3); serum/glucocorticoid regulated kinase (SGIK); N-myc downstream regulated gene 1 (NDRG1); AXIN1 up-regulated (AXUD1); and combinations thereof.

In one embodiment of the present invention, a method is provided for detecting a toxicogenomic change in gene expression in cells exposed to a nanomaterial comprising: a) generating a control cDNA or cRNA population from a population of control cells; b) contacting a test cell population with a composition comprising a nanomaterial; c) generating a test cDNA or cRNA population from the test cells after contact with the composition comprising the nanomaterial; d) contacting the control and test cDNA or cRNA populations under hybridization conditions with microarrays comprising a plurality of polynucleotide sequences that each represent genes or gene specific portions of genes, said microarray including a nanomaterial biomarker set; and e) determining a relative degree of microarray hybridization between with the control and test cDNA or cRNA; wherein an increase or decrease relative degree of hybridization with one or more of the nanoparticle biomarker set between the control and test cDNA or cRNA indicates toxicogenomic change in gene expression in cells exposed one or more components of the composition comprising the nanomaterial.

In one embodiment, a method is provided for detecting a toxicogenomic change in gene expression in cells exposed to a nanomaterial comprising: a) generating a control cDNA population from a population of control cells; b) contacting a test cell population with a composition comprising a nanomaterial; c) generating a test cDNA population from the test cells after contact with the composition comprising the nanomaterial; d) contacting the control and test cDNA populations under hybridization conditions with microarrays comprising a plurality of polynucleotide sequences that each represent genes or gene specific portions of genes, said microarray including a nanomaterial biomarker set of polynucleotide sequences representing genes or gene specific portions of genes encoding Nice-1 and kallikrein-5 and one or more additional genes selected from the group consisting of the genes identified on FIGS. 9A-C; and determining a relative degree of microarray hybridization between with the control and test cDNA; wherein an increase or decrease relative degree of hybridization with one or more of the nanoparticle biomarker set between the control and test cDNA indicates toxicogenomic change in gene expression in cells exposed one or more components of the composition comprising the nanomaterial.

In one embodiment of the invention, the biomarker set includes polynucleotide sequences representing genes or gene specific portions of genes identified on any one of FIGS. 9A-9C, FIG. 21, FIG. 22, FIG. 23 and FIG. 24.

In another embodiment of the invention, the biomarker set includes polynucleotide sequences representing genes or gene specific portions of a plurality of genes selected from the identified on any one of FIGS. 9A-9C and FIG. 24.

In one embodiment of the invention, a biomarker set is provided for identifying exposure of a cell to a nanomaterial wherein the biomarker set identifies up or down regulation of a plurality of the genes selected from the genes set out on any one of FIGS. 9A-C, 21, 22, 23 and 24. The biomarker set can be for detection of cDNA, cRNA or protein that relate directly to up or down regulated expression of the plurality of genes.

In another embodiment of the invention, a biomarker set is provided for identifying nanoparticle exposure type on the basis of relative toxicity by up or down regulation of a plurality of genes selected from the genes set out on any one of FIGS. 15 and 16.

In one embodiment of the invention, relative toxicity is identified by differential gene expression of one or more of the genes selected from the group consisting of: Homo sapiens cDNA FLJ10941 fis, clone OVARC1001243 (ACCN AK001803); Homo sapiens neurofibromin 1 (neurofibromatosis, von Recklinghausen disease, Watson disease) (NF1), mRNA (ACCN NM_{—}000267), Homo sapiens CDC-like kinase1 (CLK1), mRNA (ACCN NM_{—}004071); Homo sapiens mRNA; cDNA DKFZp56402423 (from clone DKFZp56402423) (ACCN AL390214); Homo sapiens mRNA for KIAA0624 protein, partial cds (AB014524); and Homo sapiens cDNA: FLJ22917 fis, clone KAT06430 (AK026570).

In another embodiment, a visual method for identification of nanoparticle exposure by cells is provided, including comparing GEM profiles from exposed or putatively exposed cells with GEM profiles from control cells by three dimensional display of principal component analysis data.

### BRIEF DESCRIPTION THE DRAWINGS

For a more complete understanding of the present invention, including features and advantages, reference is now made to the detailed description of the invention along with the accompanying figures:

FIG. 1 presents GEM results for SiO₂ nanoparticle exposure in HEK cells.

FIG. 2 presents GEM results for TiO₂ nanoparticle exposure in HEK cells.

FIG. 3A-C presents GEM results for CB nanoparticle exposure in HEK cells.

FIG. 4A presents expression values for Ferronyl Iron (Carbonyl Iron-Low Dose) for the genes that are predominantly down regulated at low dose. FIG. 4B presents expression values for Ferronyl Iron (Carbonyl Iron-High Dose) for the same genes in FIG. 4A that are predominantly down regulated at low dose.

FIG. 5A presents GEM results for the genes primarily up-regulated by Ferronyl iron nanoparticle exposure at low dose in HEK cells.

FIG. 5B presents GEM results for the genes primarily up-regulated by Ferronyl iron nanoparticle exposure at high dose in HEK cells.

FIG. 6A-P present GEM results for low dose SiO₂ nanoparticle exposure over time in HEK cells.

FIG. 7A-O present GEM results for high dose SiO₂ nanoparticle exposure over time in HEK cells.

FIG. 8 presents GEM results for SWNT nanoparticle exposure at high and low doses at 24 hours in HEK cells.

FIGS. 9A, B and C present summary results identifying biomarkers of nanoparticle exposure.

FIG. 10 presents MTT assay cytotoxicity curves for FC (FIG. 10A), SiO2 (FIG. 10B), SWNT (FIG. 10C) and CB (FIG. 10D).

FIG. 11 graphically depicts principal components analysis for nanomaterial exposure.

FIG. 12A1-5 presents GEM results for genes predominantly up-regulated in response to TiO₂ nanoparticle exposure in HEK cells.

FIG. 12B1:-2 presents GEM results for genes predominantly down-regulated in response to TiO₂ nanoparticle exposure in HEK cells.

FIG. 13A1-13 presents GEM results for genes predominantly down-regulated in response to CB nanoparticle exposure in HEK cells.

FIG. 13B1-17 presents GEM results for genes predominantly up-regulated in response to CB nanoparticle exposure in HEK cells.

FIG. 14A1-4 presents GEM results for genes predominantly down-regulated in response to SiO₂ nanoparticle exposure in HEK cells.

FIG. 14B1-7 presents GEM results for genes predominantly up-regulated in response to SiO₂ nanoparticle exposure in HEK cells.

FIGS. 15A and B represents LDA Analysis of the data of FIGS. 12 (TiO₂), 13 (CB) and 14 (SiO₂)

FIG. 16A-D represents QDA Analysis of the data of FIGS. 12 (TiO₂), 13 (CB) and 14 (SiO₂)

FIG. 17A1-23 presents GEM results for genes predominantly down-regulated in response to low dose CB nanoparticle exposure over time in HEK cells.

FIG. 17B1-32 presents GEM results for genes predominantly up-regulated in response to low dose CB nanoparticle exposure over time in HEK cells.

FIG. 18A1-74 presents GEM results for genes predominantly down-regulated in response to high dose CB nanoparticle exposure over time in HEK cells.

FIG. 18B1-47 presents GEM results for genes predominantly up-regulated in response to high dose CB nanoparticle exposure over time in HEK cells.

FIG. 19A1-10 presents GEM results for genes predominantly down-regulated in response to low dose SWNT nanoparticle exposure over time in HEK cells.

FIG. 19B1-7 presents GEM results for genes predominantly up-regulated in response to low dose SWNT nanoparticle exposure over time in HEK cells.

FIG. 20A1-15 presents GEM results for genes predominantly down-regulated in response to high dose SWNT nanoparticle exposure over time in HEK cells.

FIG. 20B1-39 presents GEM results for genes predominantly up-regulated in response to high dose SWNT nanoparticle exposure over time in HEK cells.

FIG. 21 depicts predictive biomarkers for nanomaterial exposure including genes significantly expressed up or down after exposure with two out of three of the three compounds, TiO₂, CB and SiO₂, or with all three based on the data presented in FIGS. 12A&B (TiO₂), 13A&B (CB), and 14A&B (SiO2).

FIG. 22 depicts predictive biomarkers for exposure to TiO₂, CB, SiO₂ and SWNT at low dose (from the time coure studies).

FIG. 23 depicts predictive biomarkers for exposure to TiO₂, CB, SiO₂ and SWNT at low dose (from the time coure studies).

FIG. 24 is cumulative of genes identified in FIG. 21; genes listed in all LDA and QDA tables depicted in FIGS. 15 and 16, and genes common to all 4 compounds from time course series at both low (FIG. 22) and high dose (FIG. 23).

### DETAILED DESCRIPTION OF THE INVENTION

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

ABBREVIATIONS: The following abbreviations are used throughout this application:

CB Carbon Black

CNT Carbon NanoTube

FBS Fetal Bovine Serum

FC ferronyl iron, a.k.a. carbonyl iron

GEM Gene Expression Microarray

HEK Human Epidermal Keratinocytes

KGM Keratinocyte Growth Medium

MWNT Multi-Walled Carbon NanoTube

NT carbon NanoTubes

OMIC genomic, proteomic, pharmacogenomic, metabolomic

PDL Population Doubling Level

SiO₂ Silica or Silicon Dioxide

SNP Single Nucleotide Polymorphism

SWNT Single-Walled Carbon NanoTubes

TiO₂ Titanium Dioxide

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

For purposes of the present invention, the term “nanoparticle” is used interchangeably with “nanomaterial” and refers to particulates on the nanometer (less than approximately 100 nm) length scale. A nanometer is one billionth of a meter (10⁻⁹ meters). Such “nanoscale” materials have very high relative surface areas, making them particularly useful in composite materials, reactive systems, drug delivery, and energy storage. Nanoparticles may be combined with other materials such as resins to form “nanocomposites.”

Considerable interest exists in carbon-based nanomaterials derived from groundbreaking research in fullerene chemistry. Nanomaterials vary greatly in size, shape and composition. Structural examples of fullerene based (carbon 60 or C₆₀) nanaomaterials include “Bucky Balls”, nanowires, nanofilms, nanocrystals (quantum dots), and nanotubes. Common nano-sized particulates include titanium dioxide (TiO₂) and silicon dioxide (SiO₂). Worldwide efforts are underway to develop and commercialize nanomaterials. However, reports on nanomaterial toxicity do not agree even as to a single chemical entity, carbon nanotubes.

In one embodiment, human cell cultures and gene expression microarrays were used in a systems biology approach in an effort to assess the risk to humans. This approach perturbs a biological system with a possible toxic insult and reiteratively samples it over time. By incorporating several time points, a more complete picture of any toxic response taking place is furnished. Particulate toxicity has been assessed by microarrays in which compounds such as silicon dioxide [SiO₂], titanium dioxide [TiO₂] and carbon black [CB], have been used as reference compounds. Wiethoff A J et al. (Inhal. Toxicol. 15(12) (2003) 1231-1246) assessed the role of neutrophil apoptosis in the resolution of particle-induced pulmonary inflammation and observed gene expression changes in rat lung tissue at 24 h postinstillation with SiO₂ However, a systems biology approach where the cells or tissues are perturbed and reiteratively-sampled over many time points and/or doses was not apparently employed.

In one embodiment, gene expression profiles of cellular exposure to nanoscale materials is provided including compiled reference profiles of nanoscale compounds previously used as controls or known toxins.

In one embodiment, exposure of cells to single-walled carbon nanotubes (SWNT) by gene expression profiling is provided including identification genes (or proteins) expressed after interaction of SWNT with human cells and comparison with similarities with known toxins.

In one embodiment, a systems biology approach is applied in order to predict cellular interactions after perturbations with an ultimate goal of creating a virtual cell. This enables “reverse engineering” of cellular pathways from data compiled after a system is perturbed and reiteratively-sampled over time and/or dose using high-throughput and efficient OMIC technologies to compile the comprehensive data.

The following examples are included for the sake of completeness of disclosure and to illustrate the methods of making the compositions and composites of the present invention as well as to present certain characteristics of the compositions. In no way are these examples intended to limit the scope or teaching of this disclosure.

### EXAMPLE I

In one embodiment, primary human neonatal epidermal keratinocytes (HEK) were treated in vitro with several nanoscale materials. These materials were used to treat randomly-proliferating HEK cultures at 8 time points ranging from 0 to 24 hr. Cell pellets were snap-frozen and stored at −80° C. Biotinylated cRNA probes were synthesized from total RNA isolated from the cell pellets and hybridized onto CODELINK Human I Bioarray microarrays containing oligomers from 9,970 unique human genes (available from GE Healthcare). Approximately 75% of the 9,970 probes passed a set of stringent quality control criteria. After image analysis, the results were analyzed by statistical methods as well as both supervised and unsupervised methods.

A preliminary experiment was performed using HEK samples treated with SiO₂, TiO₂, and CB at 1 mg/mL for 24 hr. The results from the microarrays were analyzed. Results from hierarchical agglomerative clustering (Euclidean distance metric, complete linkage) of the gene expression data showed that the overall profiles for SiO₂ and TiO₂ were more similar to each other than to the profile observed for CB.

Cell Culture: Primary neonatal human epidermal keratinocytes (HEK, Cascade Biologics, Portland, Oreg.) from a male donor were cultured in vitro in serum-free media at 37° C. and 5% CO₂. The cells were preconfluent or randomly-proliferating and at less than 10 population doubling levels at the time of treatment. The cells were seeded into culture at least 16 hours before treatment.

Compounds: TiO₂ was obtained from Sigma Chemical Company, SiO₂ (MIN-U-SIL5 from U.S. Silica Corporation), carbon black (PRINTEX 90, from Degussa Corporation). For the purposes of a preliminary gene expression profiling study, all compounds were used at 1 mg/ml (high concentration) to see if any gene expression changes would be observed.

Culture Treatment: Sets of HEK cultures were each treated with one of the compounds: TiO₂, CB and SiO₂. For each time point, four T-75 culture flasks were used for each compound in order to obtain between 2×10⁶ to 5×10⁶ cells per cell pellet. Taking into consideration 50% cell loss with these treatment concentrations (close to or at LD₅₀ levels), the optimal range of cell number should still be obtained. Cultures designated “0 hour” were cultures unexposed to any nanomaterial. The cell cultures were between 50-70% confluent at the time of treatment and were at the same population doubling level. Preconfluent cultures were used throughout the experiments to ensure that the cells would be randomly proliferating throughout the 24 hr treatment period. The study design incorporated this parameter to ensure that the metabolism of the cells did not change during treatment, which can occur if the cells reach complete confluency during this time. The treatments were done within the same experiment and with the same cell culture to assure consistency within the biological groups.

At 24 hr., the cells were trypsinized, cell counts taken and the cells snap frozen with liquid nitrogen. The culture media was saved for further analysis with assays to detect alanine transaminase (ALT), aspartate transaminase (AST) and lactate dehydrogenase (LDH). These enzyme assays would be independent monitors of toxicity and gene expression and since all enzymes are present on the microarray, levels of activity in these assays could be correlated with the level of activity on the microarray. Microtiter plates (of cultures at the same confluence as the cultures which were treated) were used in a cytotoxicity assay (MTT assay, Promega).

Total RNA Isolation: Frozen cell pellets were lysed in RNAwiz lysis reagent (Ambion) and total RNA was isolated using phenol/chloroform extraction followed by purification over spin columns (Ambion). The concentration and purity of total RNA was measured by spectrophotometry at OD260/280 and the quality of the total RNA sample was assessed using an Agilent Bioanalyzer with the RNA6000 Nano Lab Chip (Agilent Technologies).

Biotinylated cRNA Targets: Biotin-labeled cRNA was prepared by linear amplification of the Poly(A)⁺ RNA population within the total RNA sample. Briefly, 2 micrograms of total RNA were reverse transcribed after priming with a DNA oligonucleotide containing the T7 RNA polymerase promoter 5′ to a d(T)24 sequence. After second-strand cDNA synthesis and purification of double-stranded cDNA, in vitro transcription was performed using T7 RNA polymerase in the presence of biotinylated UTP.

Array Hybridization, Scanning and Image Analysis: Ten micrograms of purified cRNA was fragmented to uniform size and applied to CODELINK 10K Human I Bioarrays (9,970 unique human genes, GE Healthcare) in hybridization buffer. The Human I Bioarray contains 10,458 spotted oligonucleotides, each of approximately 30 bp embedded in a gel matrix and employs one color detection. Of these, 9,970 correspond to “Discovery” genes-unique representatives of human genes, while the remainder are in the following categories: positive controls, negative controls, fiducial and other. Positive controls are probes which will give a positive signal and are usually nonhuman and noncoding. Negative controls are probes which give a negative (no) signal and are usually nonhuman and noncoding. They are used to decide how much fluorescence is associated with the background of the array. Fiducial probes are probes which will always give a signal and are used to align the grid placed over the microarray for the scanning step and to perform image analysis. “Other” is a miscellaneous category of other control probes for mismatch base pairing and masked genes. For experimental purposes, only the Discovery genes which are found in databases such as GenBank and SwissProt were used. Other microarrays known to those skill in the art are expected to be suitable.

Arrays were hybridized at 37° C. for 18 hr in a shaking incubator. Arrays were washed in 0.75X TNT (Tris-NaCl-Tween 20) at 46° C. for 1 hr and stained with Cy5-Streptavidin dye conjugate for 30 min. Dried arrays were scanned with a GENEPIX 4000B (Axon) scanner. Data is initially image analyzed and normalized to the mean intensity of the array using CODELINK (GE Healthcare) and GENESPRING software (Silicon Genetics). To compare individual expression values across arrays, raw intensity data (generated from CodeLink Expression software) from each gene was normalized to the median intensity of the array. Only genes that have values greater than background intensity in at least one condition were used for further analysis.

Data Analysis: After quality checks with controls, control oligos are deleted from further analysis. Only data from Discovery genes are further analyzed. The data is analyzed to exclude quality flags of C, I, L, and S (C=background contamination of the spot; I=irregular shape of the spot; L=low signal or near background; and S=signal of the spot was saturated). Genes whose signal was masked, had any flag or null annotation were excluded from analysis. Only genes with expression signals of “G” (good signal) over all time points and doses are kept. A “good” quality flag is characterized by having a signal that is “good” and within in specifications. The array is scanned and the negative control probes are analyzed for the background signal of the array itself. All negative control probes are analyzed and the outliers discarded. The resulting set is used to compute the mean negative control and this value is normalized over the entire array to get the normalized trim mean negative control. A probe with a “G” quality flag is one which has passed the threshhold set by the normalized trim mean negative control, is above the calculated background and has a regular spot shape.

The biological samples are run in triplicate and the expression values from the triplicate arrays for each compound, timepoint, and dose are averaged and any standard deviations over 10% are checked and the outlying value excluded from the average. All values for untreated 0 hr timepoints for both doses are averaged together, since these cultures were untreated with any compound. Each average gene expression value is divided by its 0 hr control. All expression values greater than 2-fold upregulated or greater than 2-fold downregulated are considered significant. Further analysis by unclassified and classified mathematical modeling algorithms (clustering, Principal Component Analysis [PCA], Integrated Bayesian Inference System [IBIS], Sub-Linear Association Mining [SLAM]) were performed with Improved Outcomes Software GENELINKER Platinum (version 4.5).

These experiments were performed according to the MIAME guidelines (Brazma, A., et al. Nat. Genet. 29(4) (2001) 365-371), which suggest basic guidelines for gene expression microarray experiments. However all experimental design and process parameters have been optimized for enhanced predictability.

### EXAMPLE II

### Optimized Parameters for Design of Expression Profiling

Cell Culture: Master and working cell banks are made to ensure that there are enough cells from the same donor to do all treatment experiments with. Cells are treated at the same time each day of treatment to avoid interference, if any, from circadian rhythm. Cells are treated within a tight range of days if treatments must occur over several days due to limited personnel resources or incubator space. Optimally, all treatments would be done in the same time cycle.

Media, tissue culture flasks, reagents and pipets for the treatments will be from the same lot, as possible. Reagents and cultureware should be certified sterile, if necessary, and if to be used under sterile conditions, checked periodically for contamination. Incubator levels of water, atmosphere (% CO₂) and temperature are checked daily and recorded.

Cells are treated at the same growth phase. The same percentage of confluence is used to avoid variability in growth parameters and metabolism rates. The same cell population doubling level (or cell passage) is maintained throughout all treatments. The optimum range is 0-1 PDL difference. If working with cell lines, the same parameters apply and the same lot from the distributor is used for all experiments. The cells are contamination-free and checks for mycoplasma, bacterial, fungal and mold contamination are made during the various phases of cell culture (cell banks, routine culturing, experimental treatments).

The cells are characterized by visual observation, cytotoxicity assays, cell density experiments and independent enzyme assays. These additional assays and experiments are performed before the treatments to set optimal conditions for each cell type, line or culture. Cytotoxicity and enzymes assays may be used as independent monitoring of cell function alongside gene expression experiments. All enzyme assays use enzymes (or proteins, genes) which are represented on the microarray.

Time points are closely monitored to adhere as tightly as possible to the established time. point. The actual experimental time point does not differ by more than 5 minutes from the established scheduled time point. Enzyme and cytotoxicity incubations steps should occur within 2-3 minutes of the established scheduled time points. Deviations from these parameters and any observations that are not expected are recorded. Optimally, the same model or serial number of laboratory and culture equipment is used to maintain consistency.

Preferably, the same technician should perform the experiments from one treatment cycle to the next. Optionally, in the case of multiple personnel, the same technician is assigned to the same experimental steps from one treatment cycle to the next. Limiting the numbers of personnel performing the various experimental steps decreases variability due to differences in technical expertise.

Experiments using Mammalian Animals: Animal husbandry conditions (no. of animals per cage, bedding, water, food, temperature and lighting conditions, controlled to minimize variability) are maintained the same throughout all treatments. Treatments are done at the same time every day if a range of treatment days is necessary to avoid interference from circadian rhythm. The same vehicle (solvent) is used for control animals as animals which are treated. Tissues from animals treated with vehicle are harvested at the same time as tissues from animals treated with compound (vehicle-matched controls). All experiments are performed on the same sex of animal unless both sexes are incorporated into the experimental design. Attention is paid to maintain consistency of litter mates whether using inbred or outbred lines. Animals from the same strain (and/or litter) should preferably be used as well as the same age throughout the experiments. The appropriate quarantine conditions (as set by ALACC certification) are used upon the arrival of the animals to ensure that they are healthy to undergo the treatments. The veterinarian in charge will set the quarantine conditions and be responsible for releasing the animals for experimental treatment.

Compounds: Compounds should be purchased of as high a purity as possible and stored as recommended by the manufacturer. If the compounds are atmospheric or light sensitive, precautions to avoid degradation if there is exposure should be taken. For example, a compound which is air-sensitive should be stored under a high purity inert gas. Also, if a compound is white light-sensitive, it should be handled under a different color light to avoid degradation and increase in impurities. Full characterization of the compounds prior to treatments is recommended including complete solubility testing. The compounds utilized formed a homogenous particulate suspension, in which the suspensions eventually settled out as precipitates.

The solvent used should be as compatible as possible with cells or animals and not cause any adverse effects. If mild adverse effects are unavoidable, recording of preclinical signs and observations should be made and vehicle matched controls should be incorporated into the experimental design for expression profiling. The expression due to the vehicle will be subtracted out from the expression of the compound under study. Stock solutions should be made immediately prior to the start of treatments. Alternatively, full characterization of the compound under these conditions will need to be made to ensure complete compound integrity at the start of treatments. Cytoxocity assays for culture experiments are conducted for characterization of the compound as well as choice of appropriate doses for the treatments. Compounds were evaluated for cytotoxicity in a MTT assay. Nontoxic and toxic doses were taken from resulting cytotoxicity curves. Methyl methanesulfonate (MMS) was evaluated alongside as a known toxic compound. These assays should be run under as many of the same experimental culture conditions as possible.

Culture Treatment: The design should include enough samplings of cells or tissues to ensure enough material at each harvest point. Enough material is necessary to run at least 3 microarrays and extra for repeat if needed. If toxicity is anticipated, enough remaining cells for at least 3 arrays plus a repeat set of 3. The same number of cells and flasks to be treated should be consistent among experimental groups. Cell counts and media supernatants taken for later characterization of enzymes should be done at each harvest point. The cells should be harvested under the same conditions each time and the approximate time of workup for each time point should be the same. The cells should be rapidly pelleted and snap frozen in liquid nitrogen to avoid degradation of RNA.

Total RNA Isolation, Biotinylated cRNA Targets, Array Hybridization, Scanning and Image Analysis: All procedures and reactions are tightly monitored and recorded. The total RNA purity and quantity is checked before the biotinylation procedure. Biotinylated targets are checked for quality and quantity. All microarrays, reagents and buffers should be of the same lot. All microarrays are quality checked before use for spot consistency and to make sure no anomalies occurred during printing. The spots should be of good round shape and consistent in quantity of probe, size and shape. All procedures for printing should include strict adherence to avoiding the exposure to lint, dust or any other environmental contamination. The same amount of target is applied to each array. Hybridization, washing and scanning steps should occur at the same time for each experimental group. The same scanning parameters and image analysis parameters are to be used with each experimental batch. The resulting flat files and array images should be ultimately archived for future reference.

Data Analysis: A complete statistical analysis of the resulting array data should be done. The reproducibility and variance within an array, between arrays of the triplicate set, between arrays of the experimental group and across all experimental groups should be made. The same preprocessing, filtering and normalization steps of the data should be consistent between and within experimental groups. Different analytical methods may required different preprocessing, filtering and normalization parameters but these parameters should be the same each time a particular analytical method is used. As much as possible characterization of various experimental parameters should be done to assess whether any variation observed is procedural or biological.

### EXAMPLE III

### Time Course Experiments

Timeline Experiments using 0, 2, 4, 6, 8, 12, 18 and 24 hr time points were conducted. The cell culture was the same as above except the population doubling levels (PDL) were kept between PDL11 and 11.5. Cells from the same donor were cultured into cell banks and frozen at PDL 11±0.5 PDL.

Compounds: The same containers and lots for CB and SiO₂ were used in these experiments. Two new compounds were used: Carbonyl iron (ferronyl iron, FC, Degussa Coproration) and Single-walled carbon nanotubes (SWNT). SWNT were manufactured using a modified chemical vapor deposition method (CoMoCAT) involving disproportionation of CO on a silica-supported Co and Mo catalyst in a tubular fluidized bed reactor (developed at Oklahoma University and commercialized by SouthWest Nanotechnologies [SWeNT]). Using this method, heavy metal impurities are very low and results in only 2 configurations of SWNT species.

The table below depicts the mean particle size of each compound. CB, SiO₂, FC, and TiO₂.

| Compound | Designation | Trade Name | Mean Particle Size |
|---|---|---|---|
| Titanium Dioxide | TiO₂ | Ti(IV)O₂ | 25 nm |
| Carbon Black | CB | Printex 90 | 14 nm |
| Carbonyl Iron | FI | Ferronyl Iron | 5.88 μm |
| Silica, α-Quartz | SiO₂ | Min-U-Sil ® 5 | 1.6 μm |
| Single-walled | SWNT | | 0.8 nm (dia) |
| carbon | | | |
| nanotubes | | | |

Prior to these experirnents, all compounds were assayed for cytotoxicity of HEK using the MTT assay. Two doses for each compound were identified: nontoxic and toxic (approximately LD₅₀.

| Compound | Non-Toxic Dose | Toxic Dose |
|---|---|---|
| FC | 0.03 mg/ml | 1 mg/ml |
| CB | 0.01 mg/ml | 0.5 mg/ml |
| SiO₂, α quartz | 0.1 mg/ml | 1 mg/ml |
| SWNT | 0.001 mg/ml | 1 mg/ml |

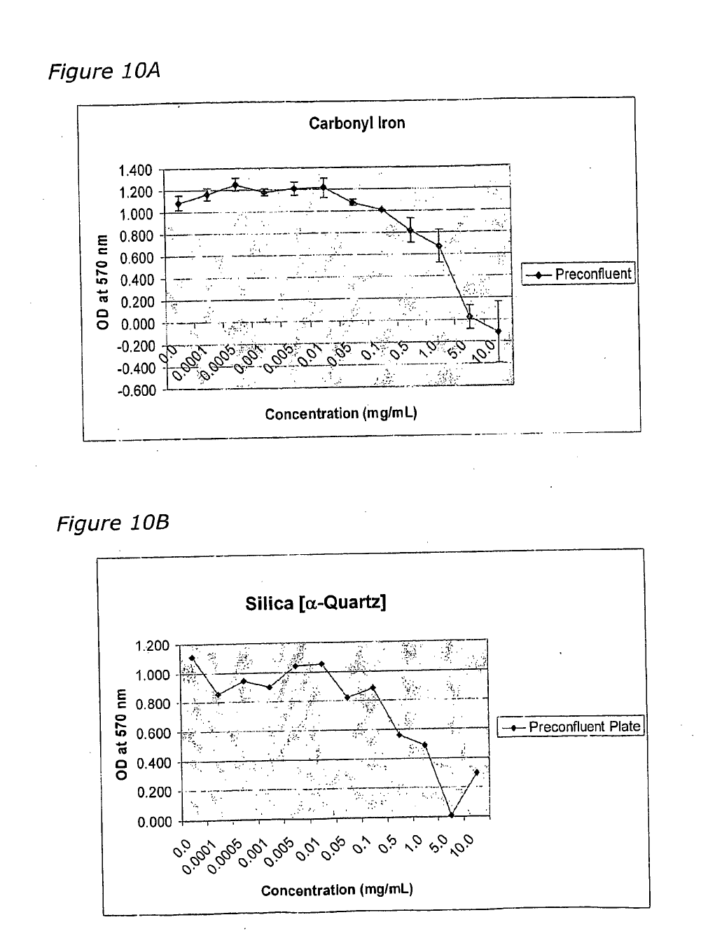

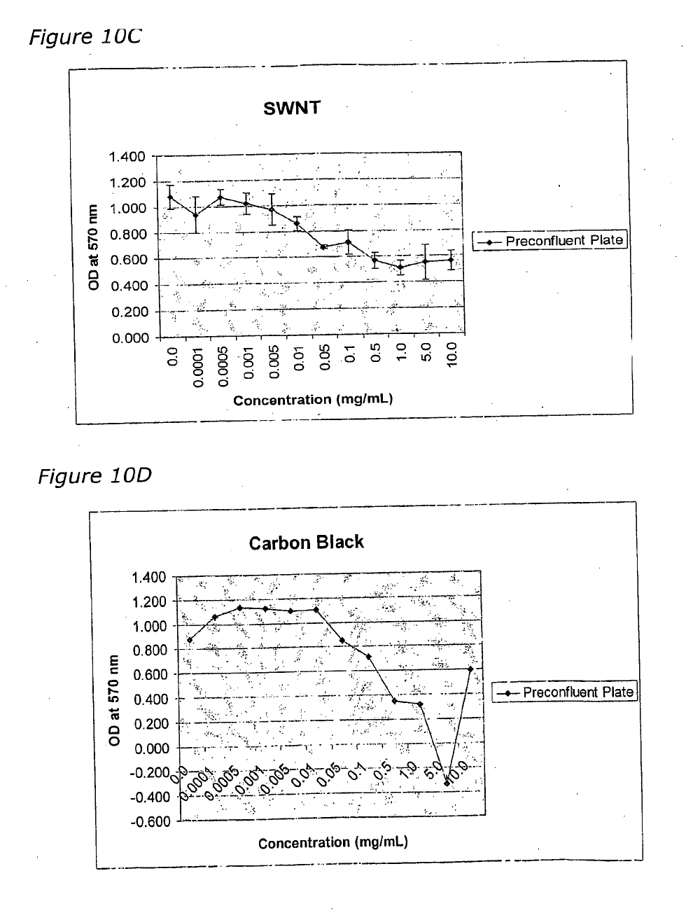

Cytoxicity curves obtained with FC (FIG. 10A), SiO₂ (FIG. 10 B), CB (FIG. 10D) and SWNT (FIG. 10C) are presented in FIG. 10.

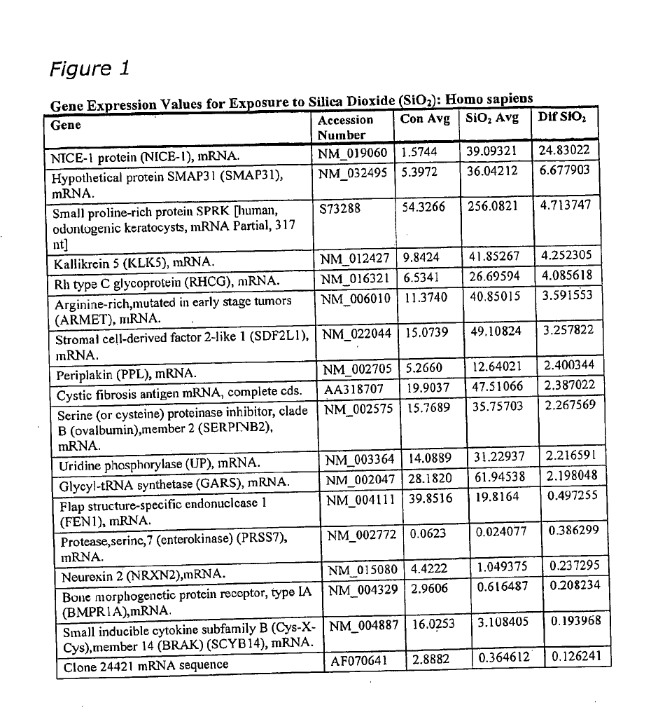

The results for SiO₂ exposure at a toxic dose of 1 mg/ml for 24 hours are presented in FIG. 1.

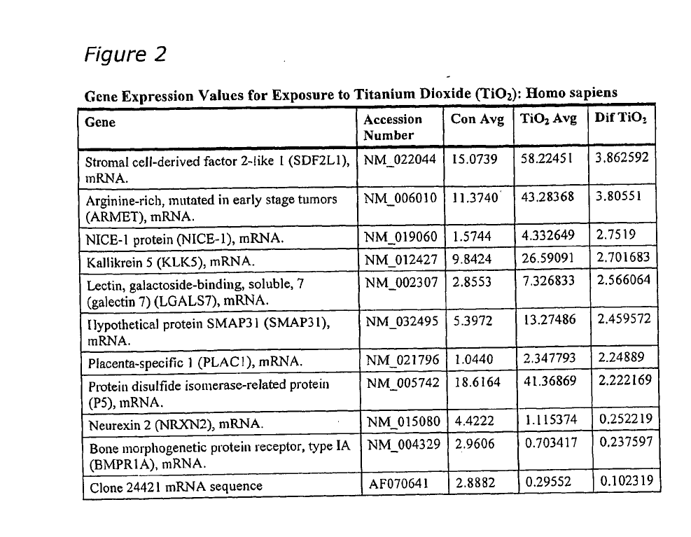

The results for TiO₂ exposure at a toxic dose of 1 mg/ml for 24 hours are presented in are presented in FIG. 2.

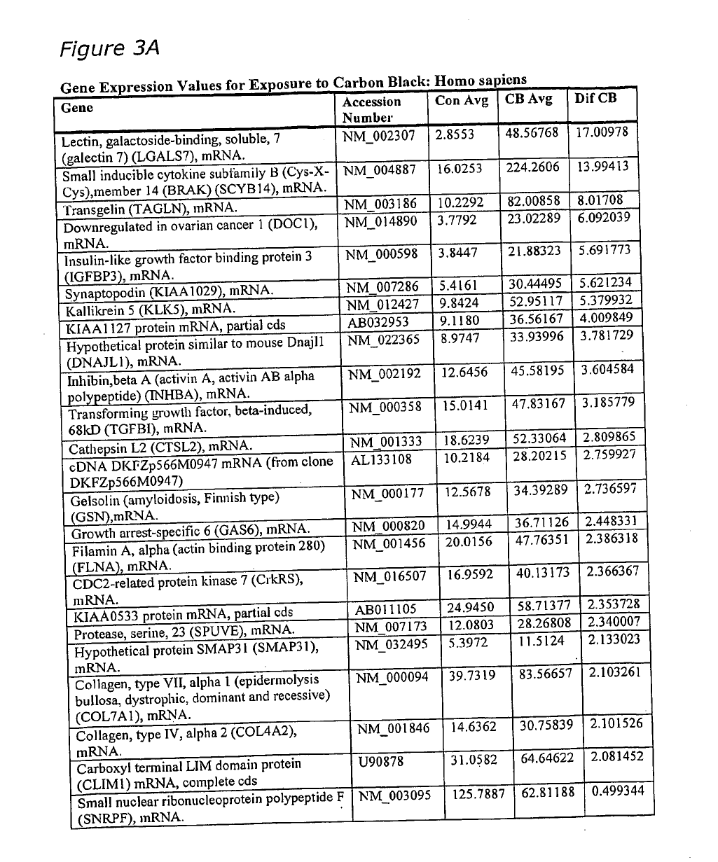

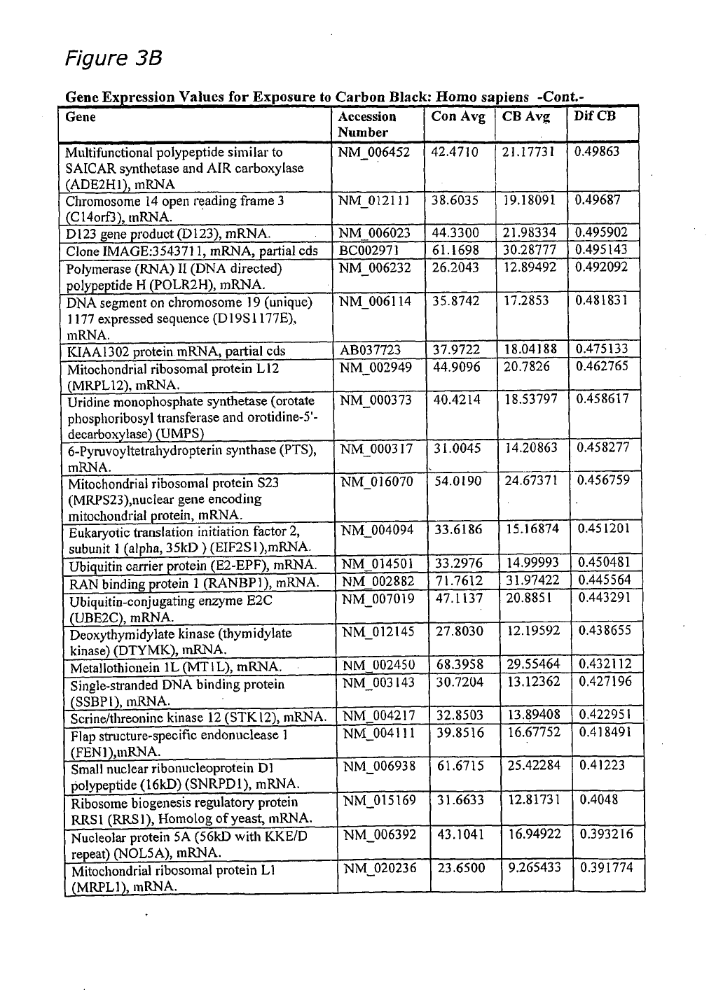

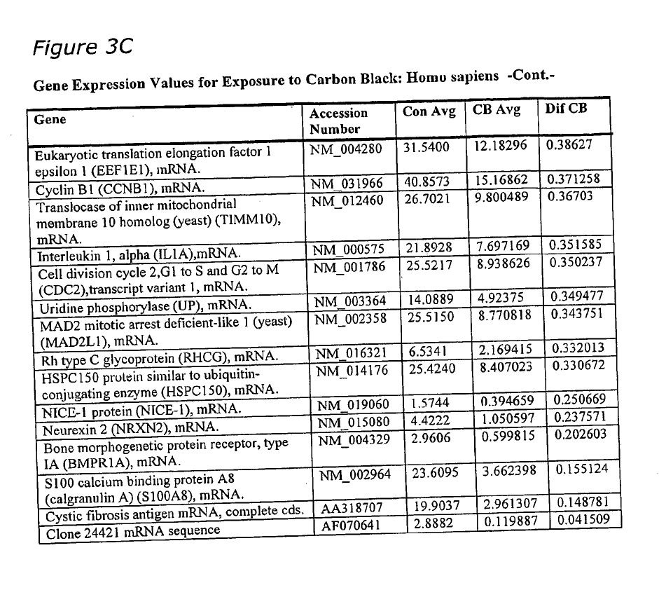

The results for CB exposure at a toxic dose of 1 mg/ml for 24 hours are presented in FIG. 3A-C.

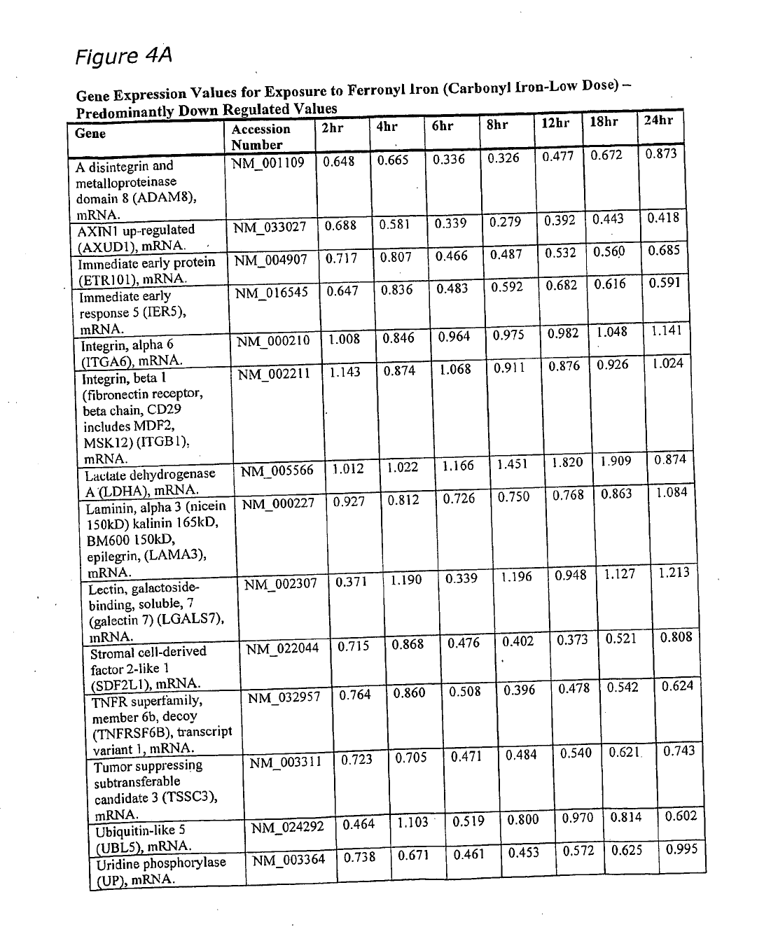

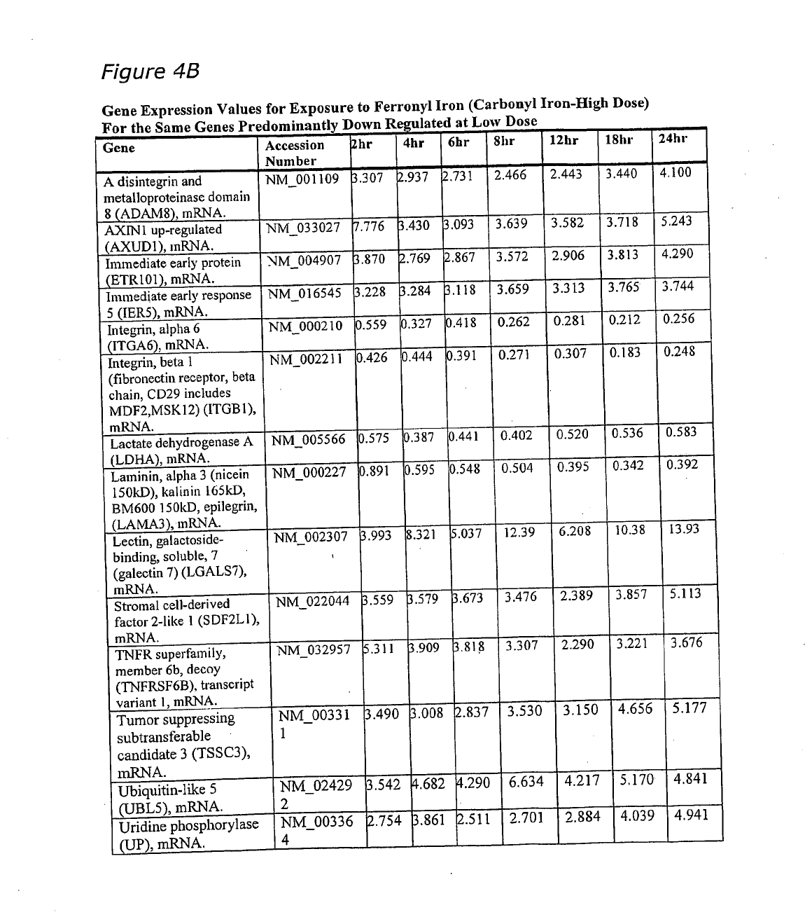

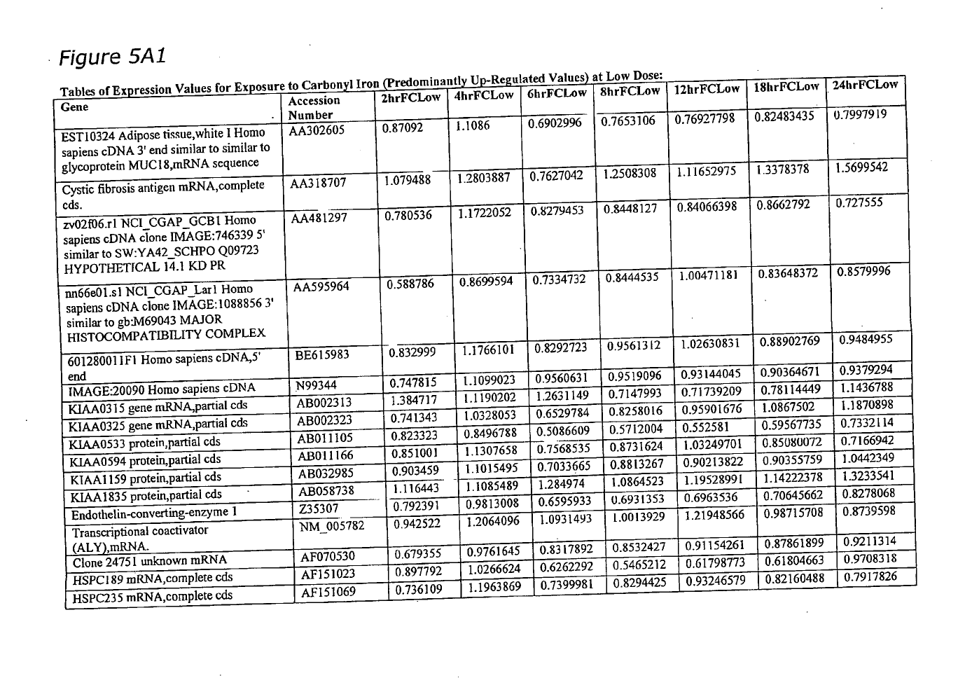

The genes primarily down regulated by exposure to ferronyl iron at low dose (0.03 mg/ml) and over time are presented in FIG. 4A. FIG. 4B presents expression values for Ferronyl Iron (Carbonyl Iron-High Dose) for the same genes in FIG. 4A that are predominantly down regulated at low dose.

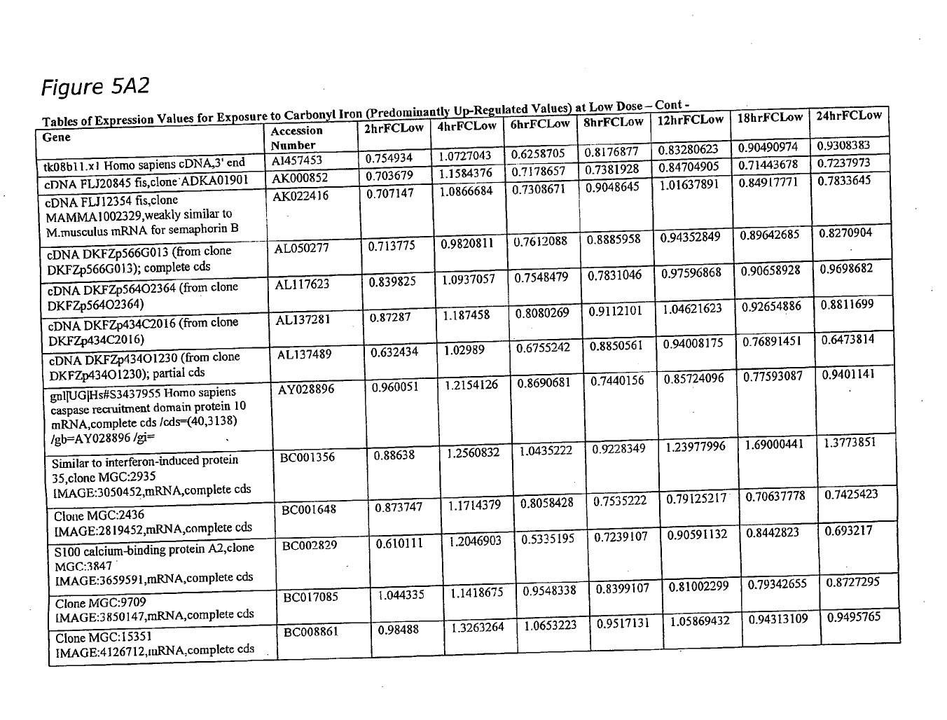

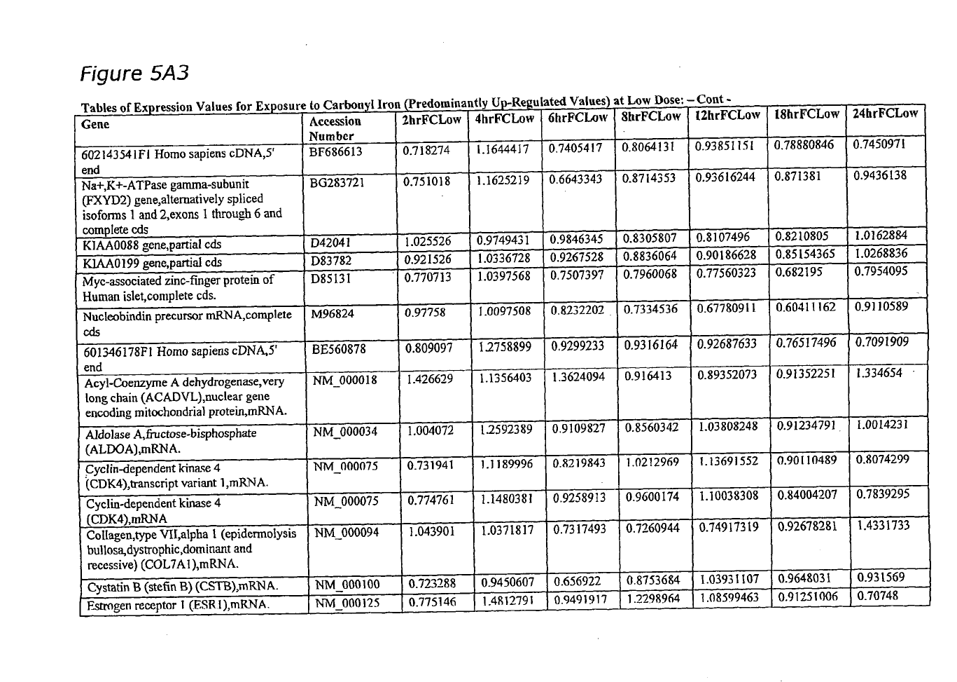

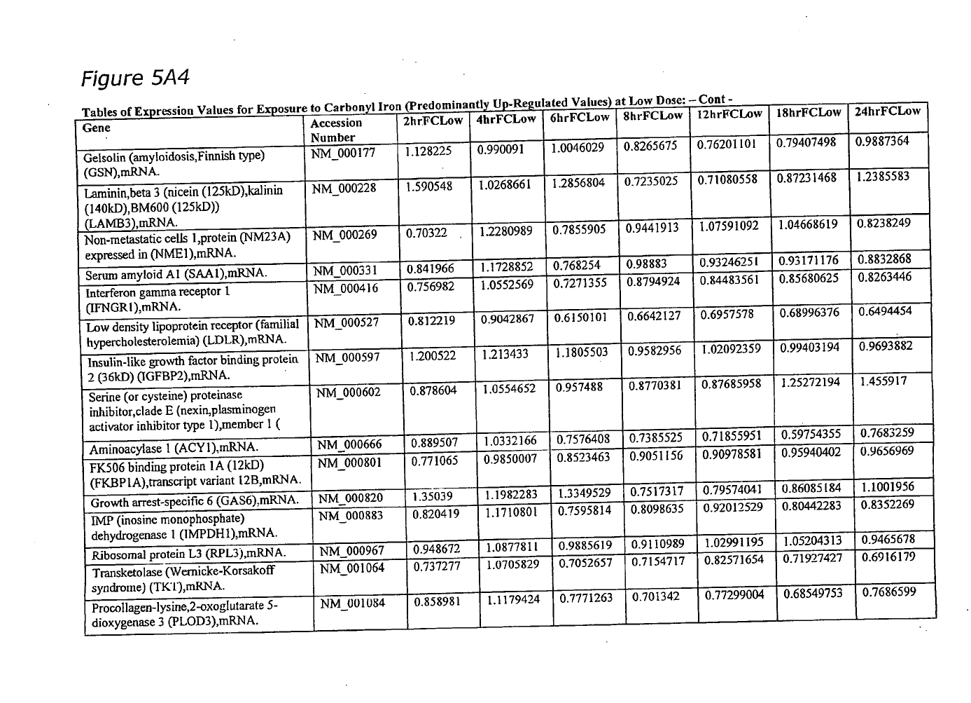

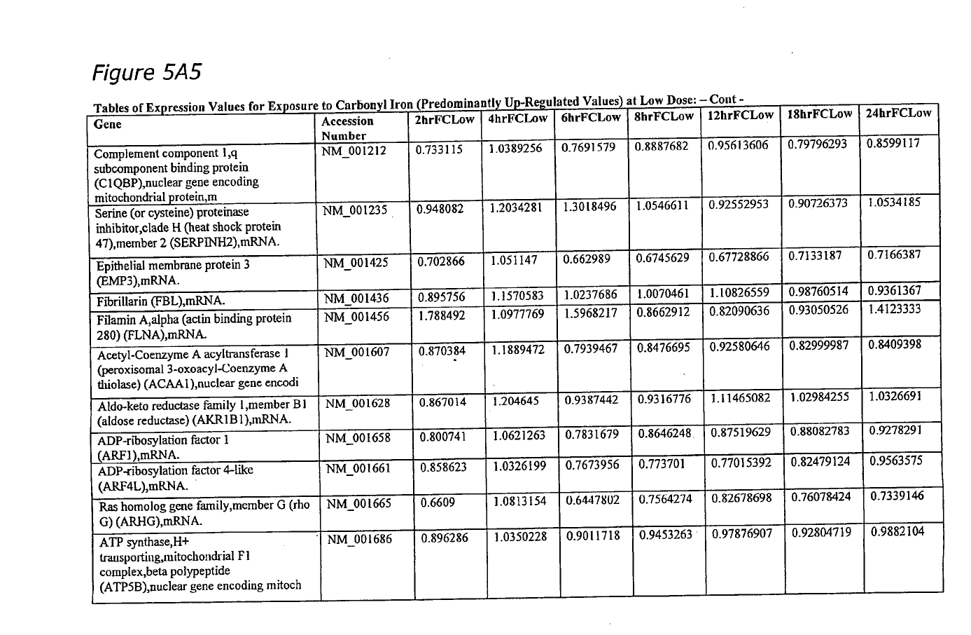

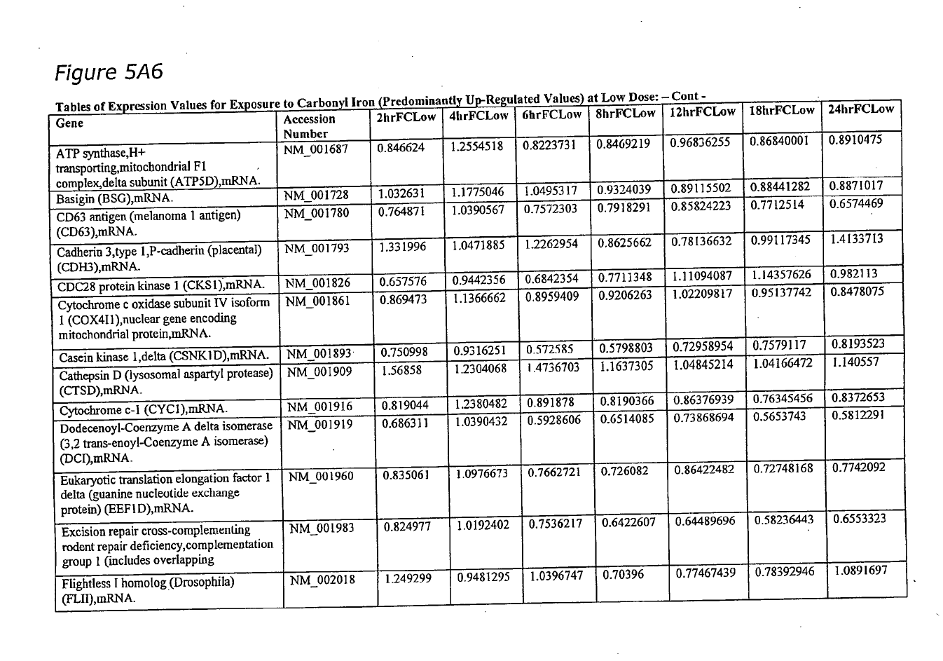

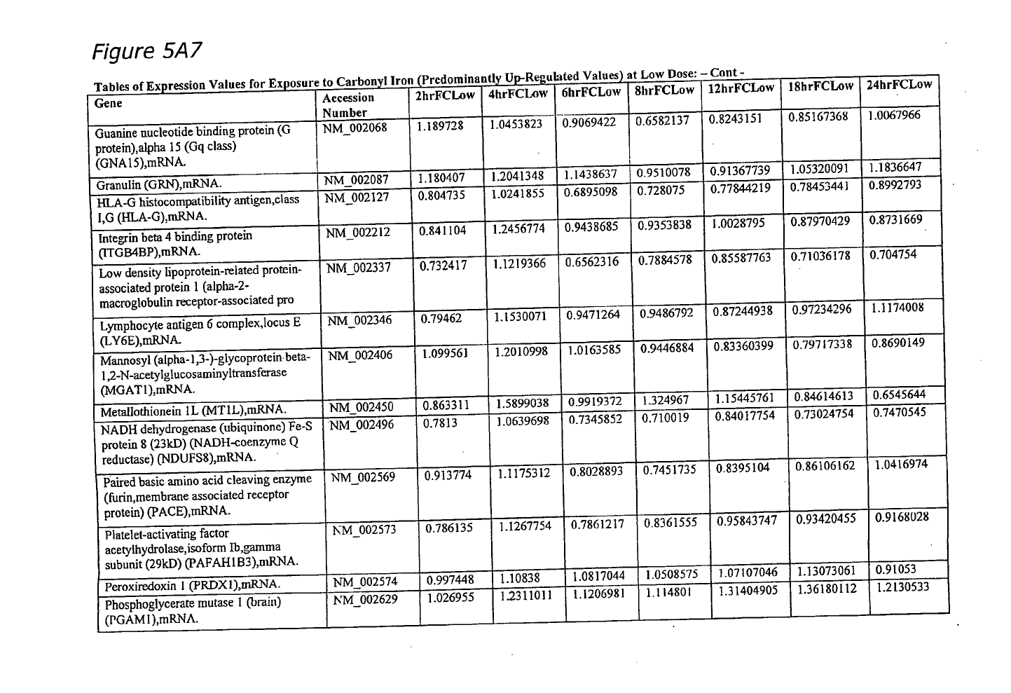

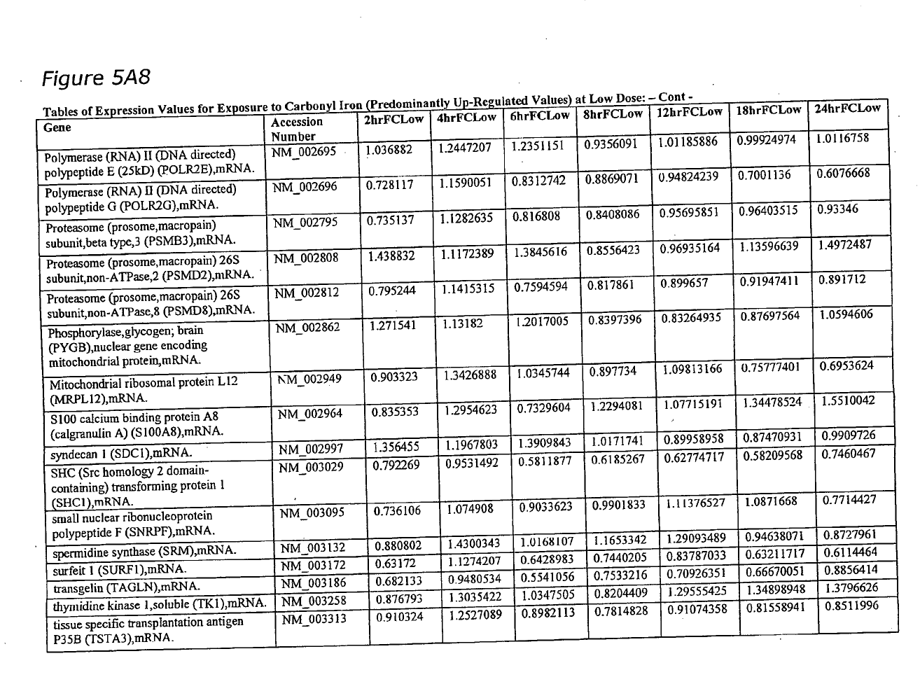

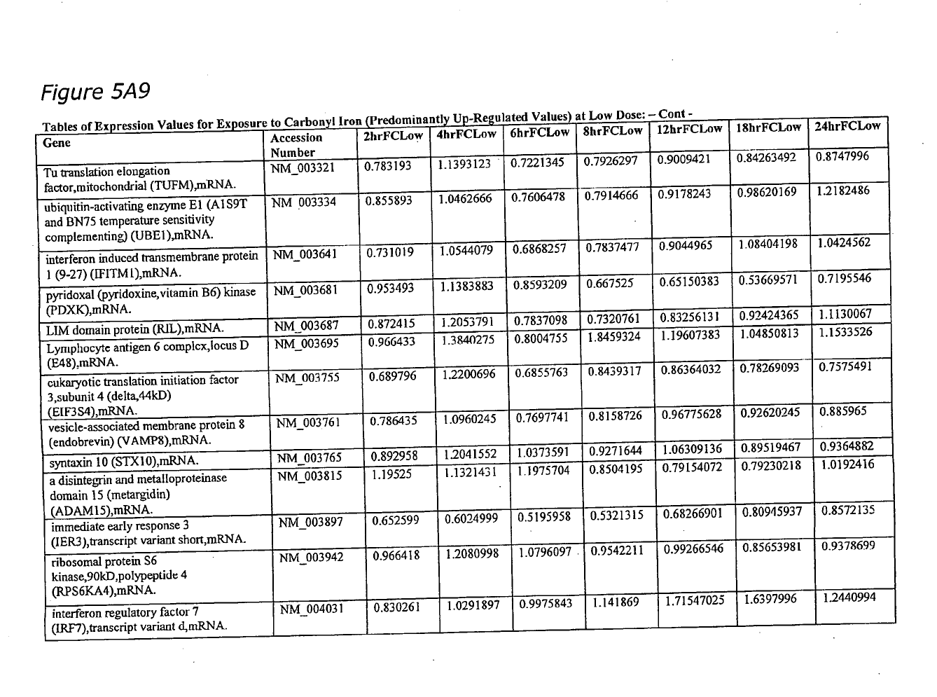

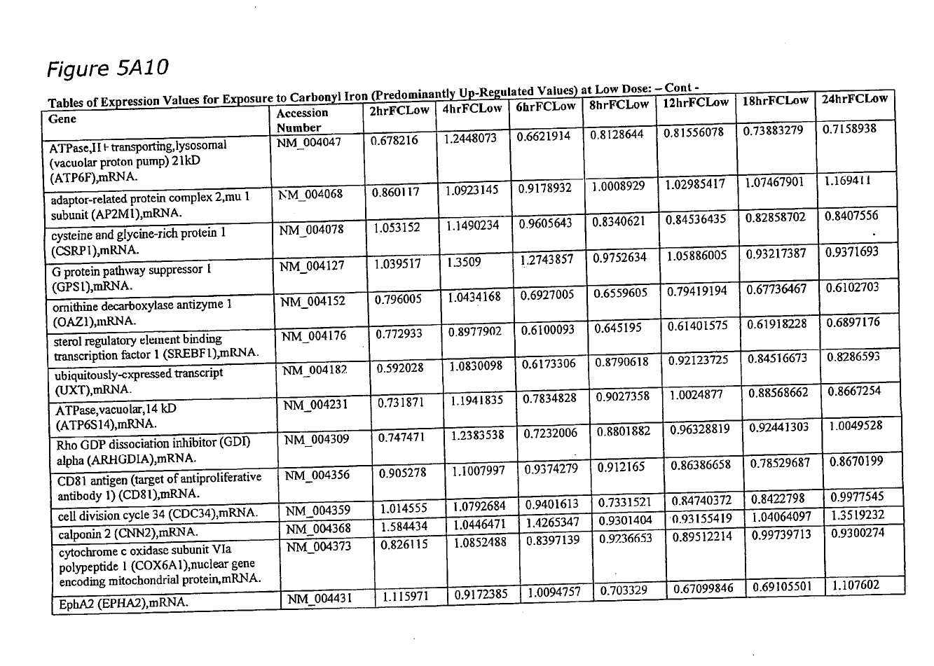

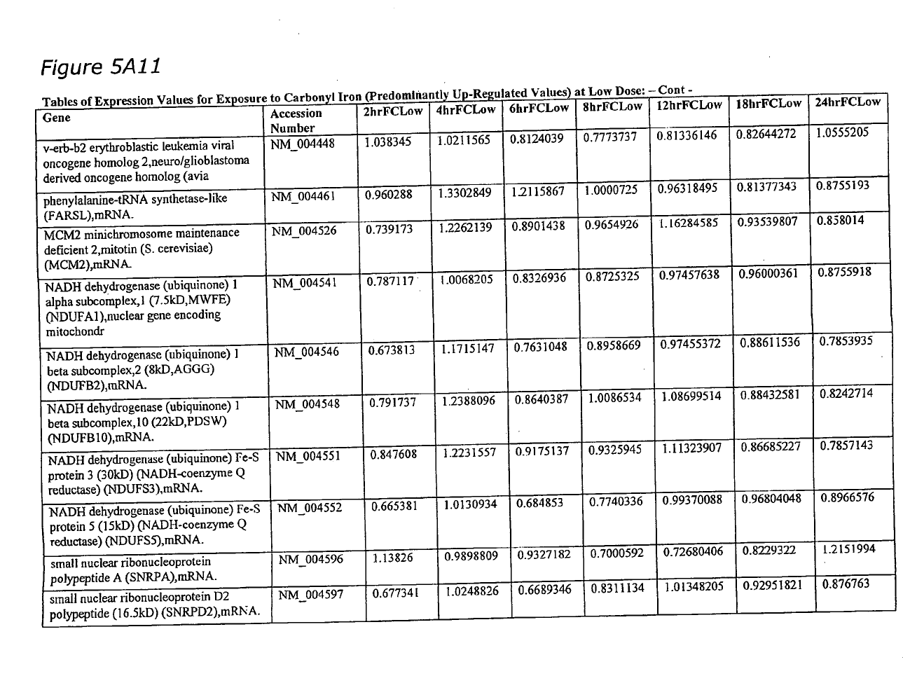

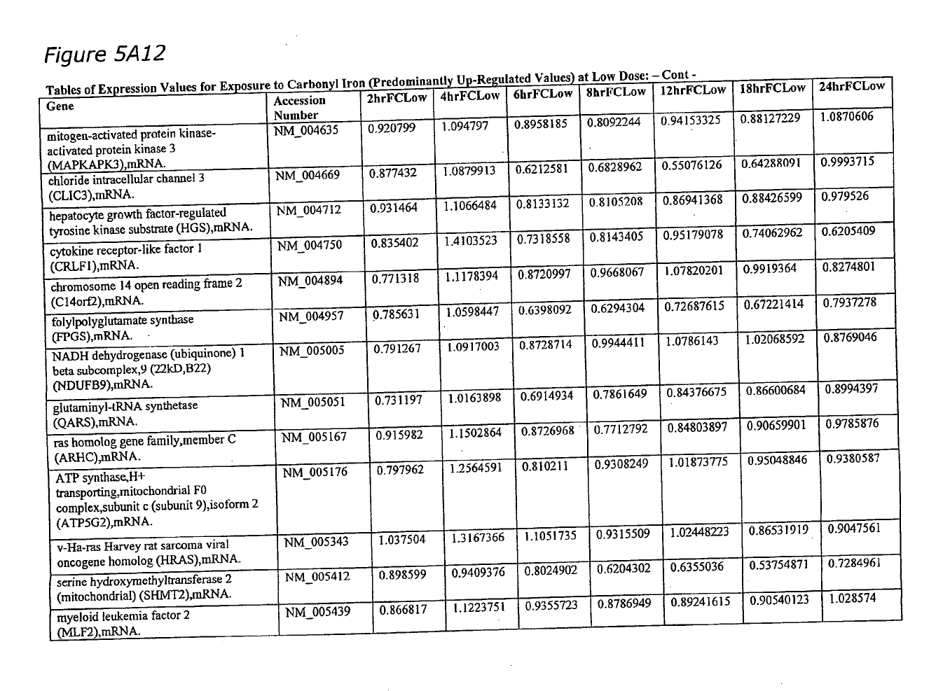

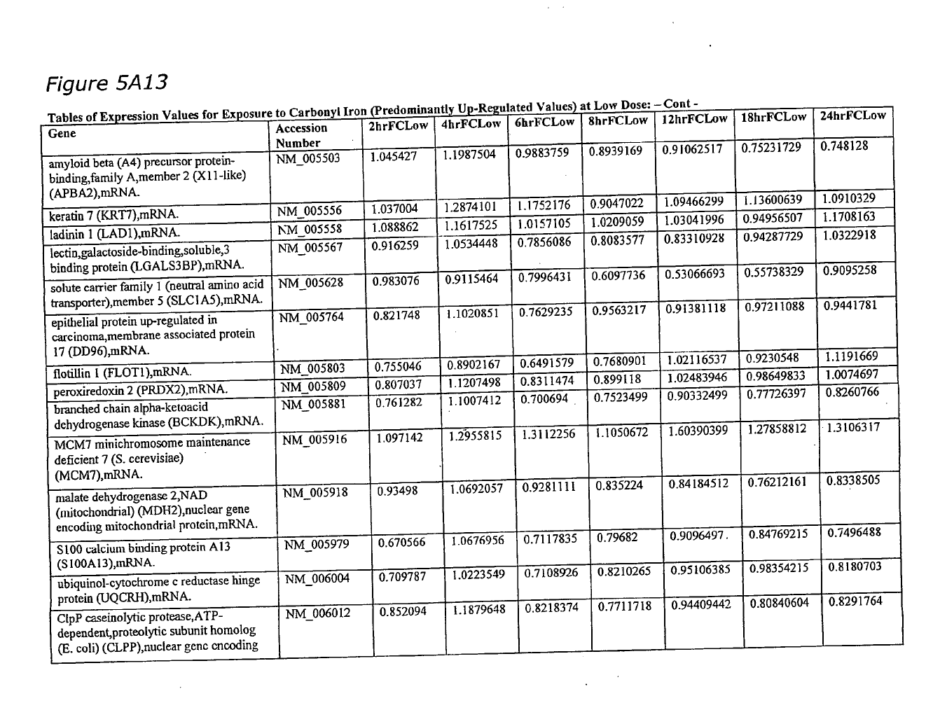

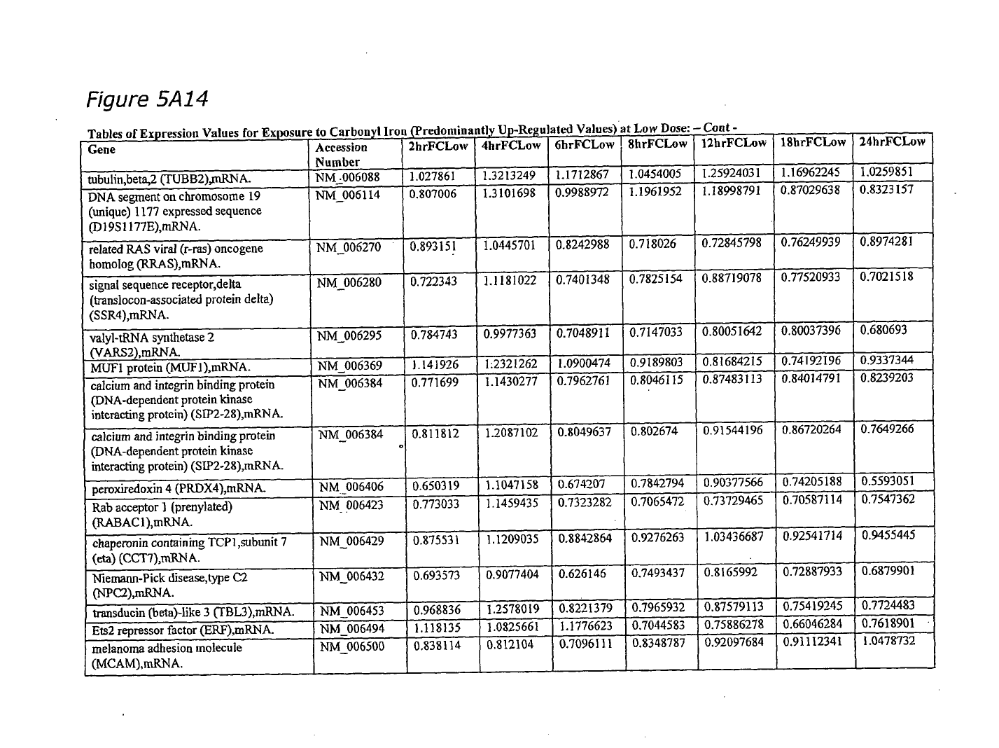

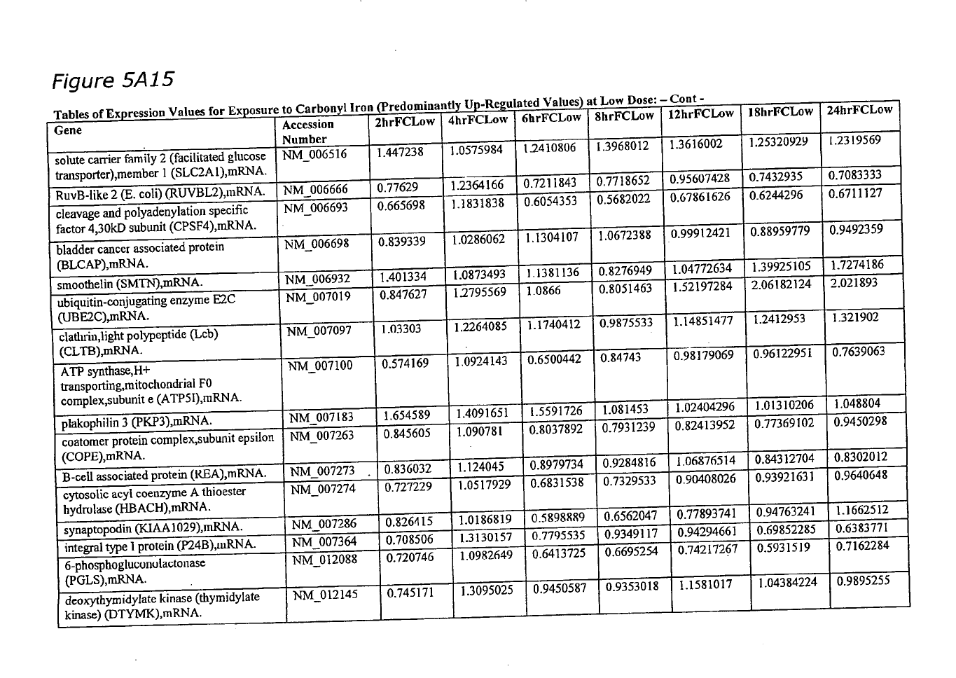

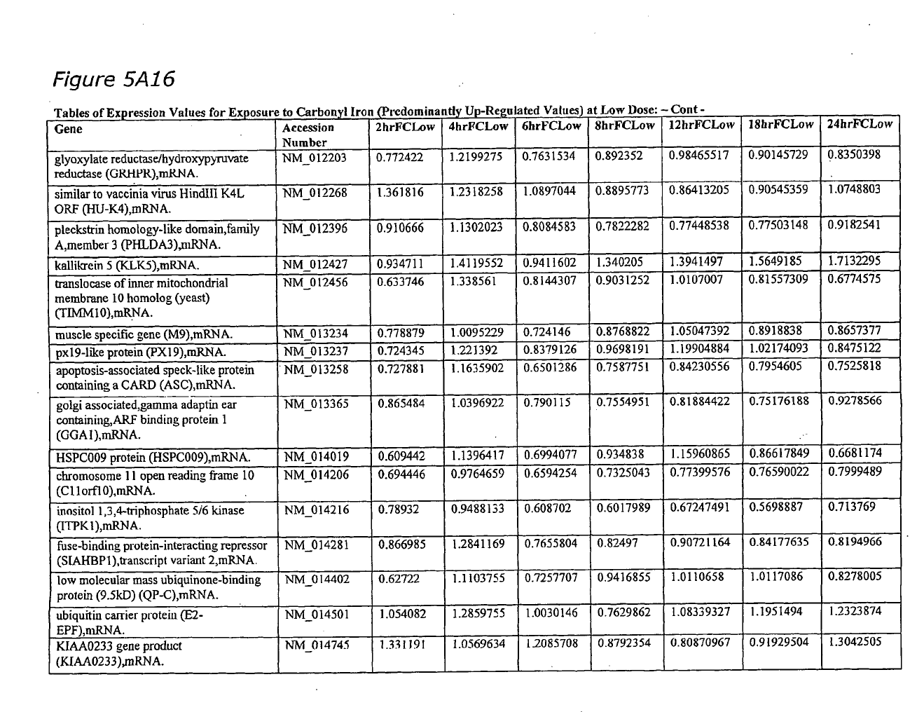

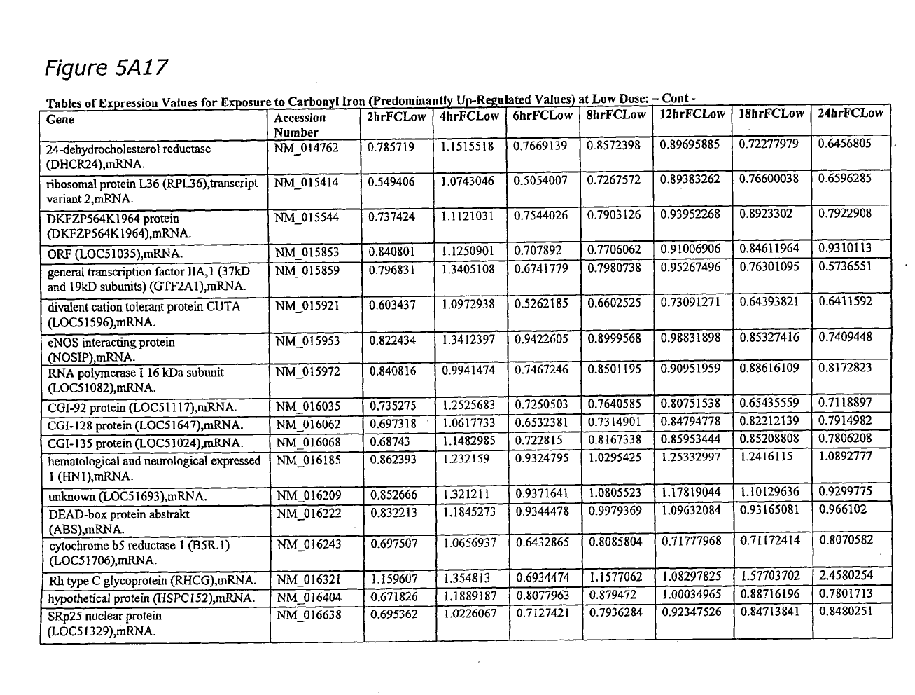

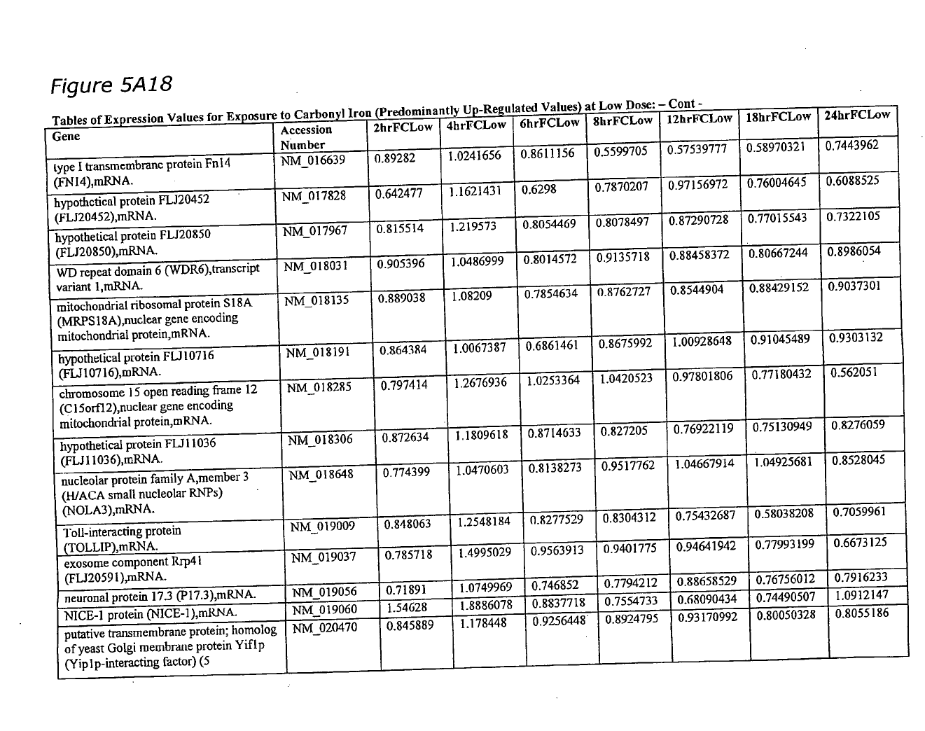

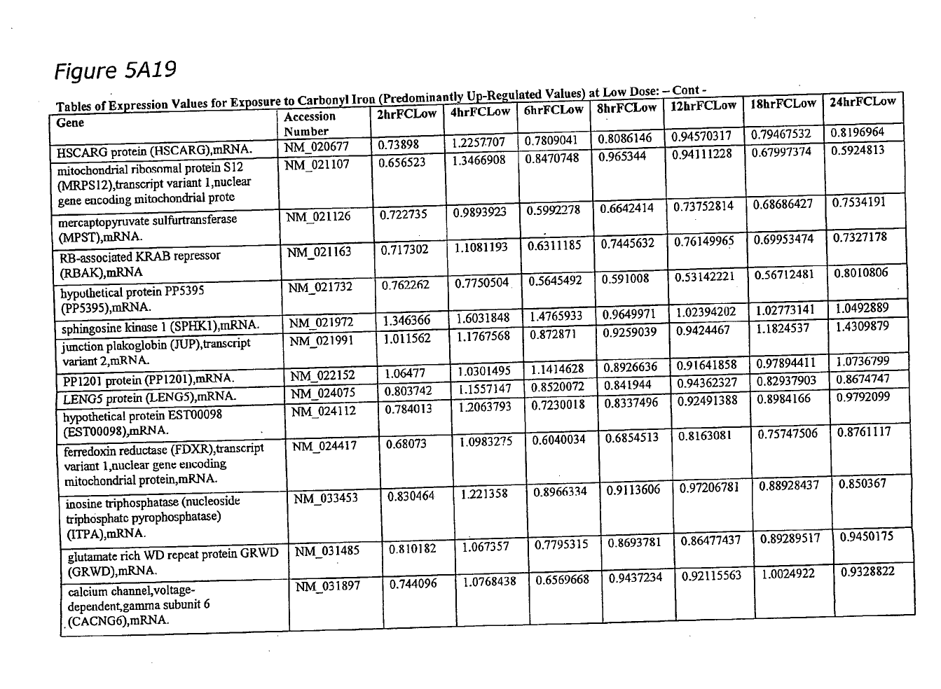

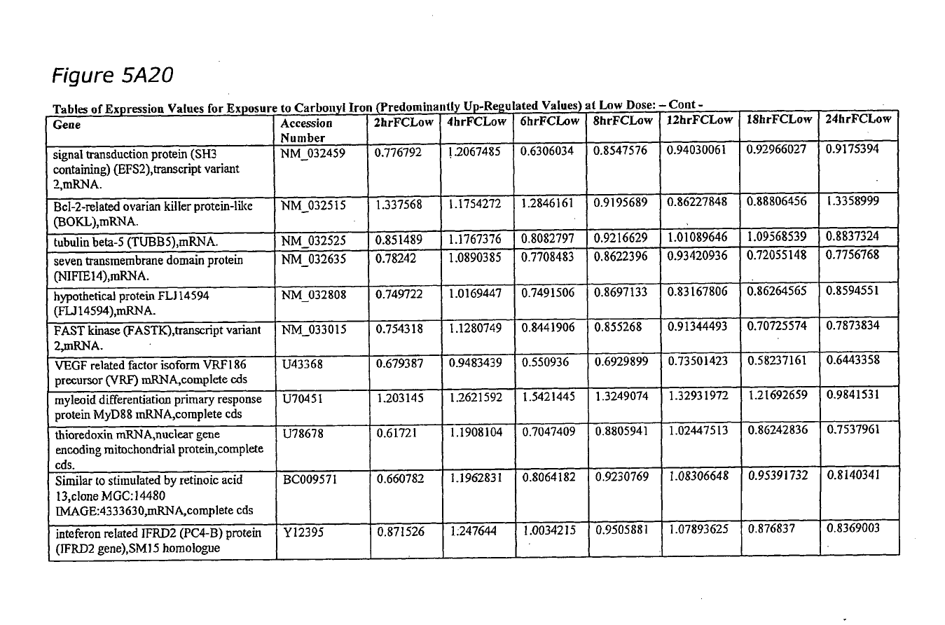

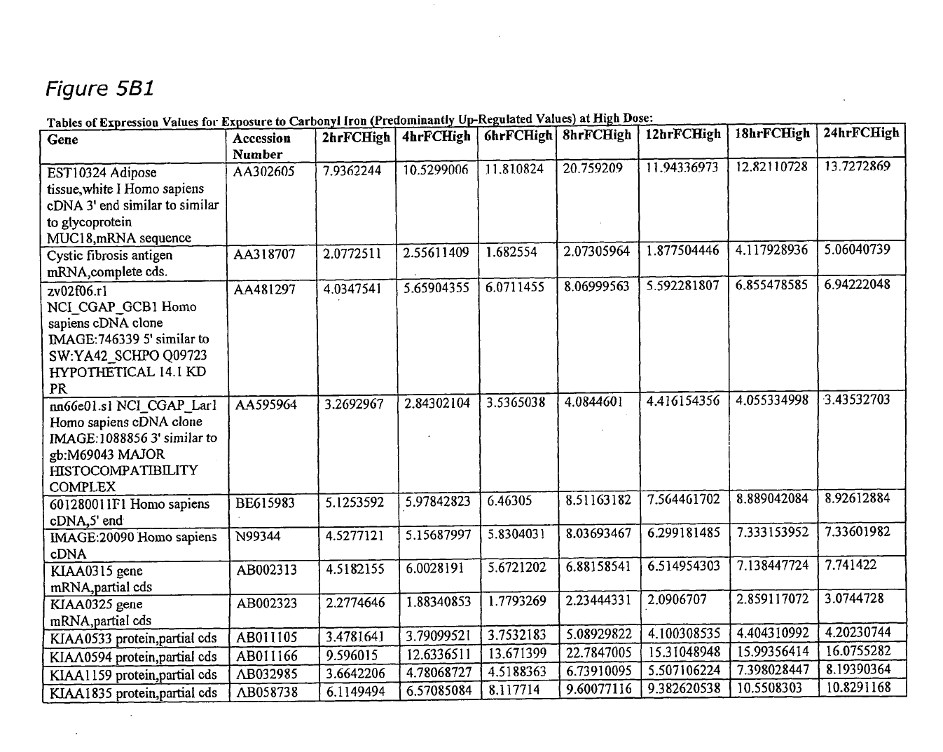

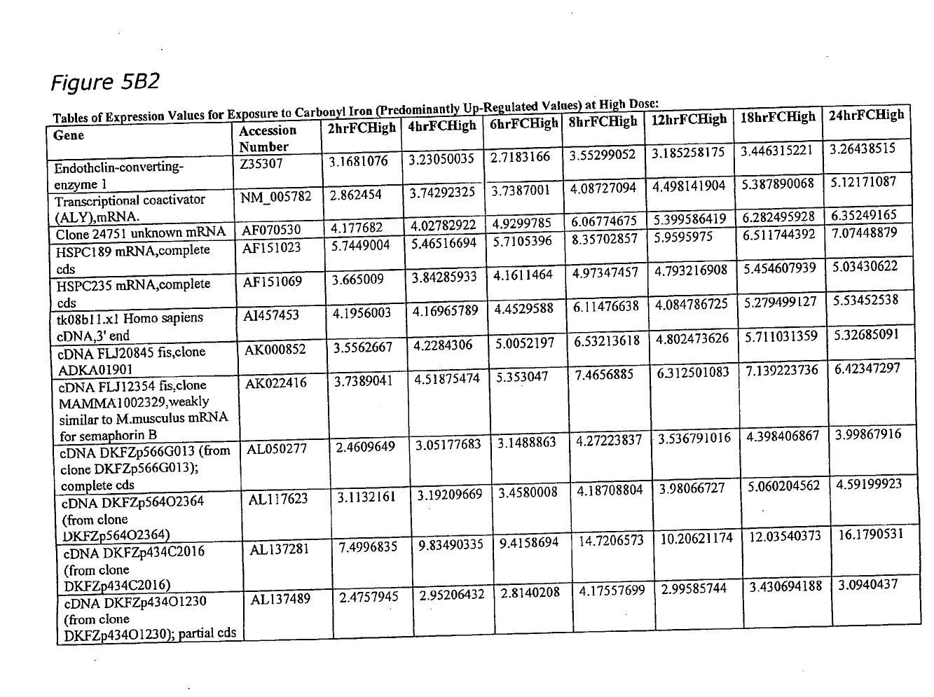

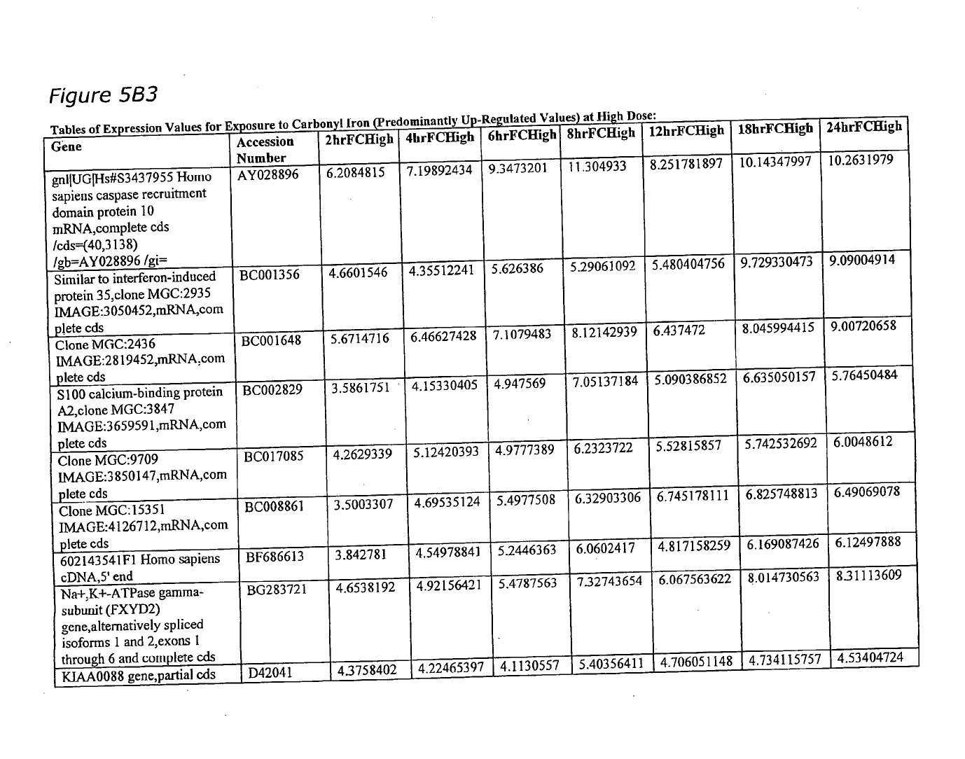

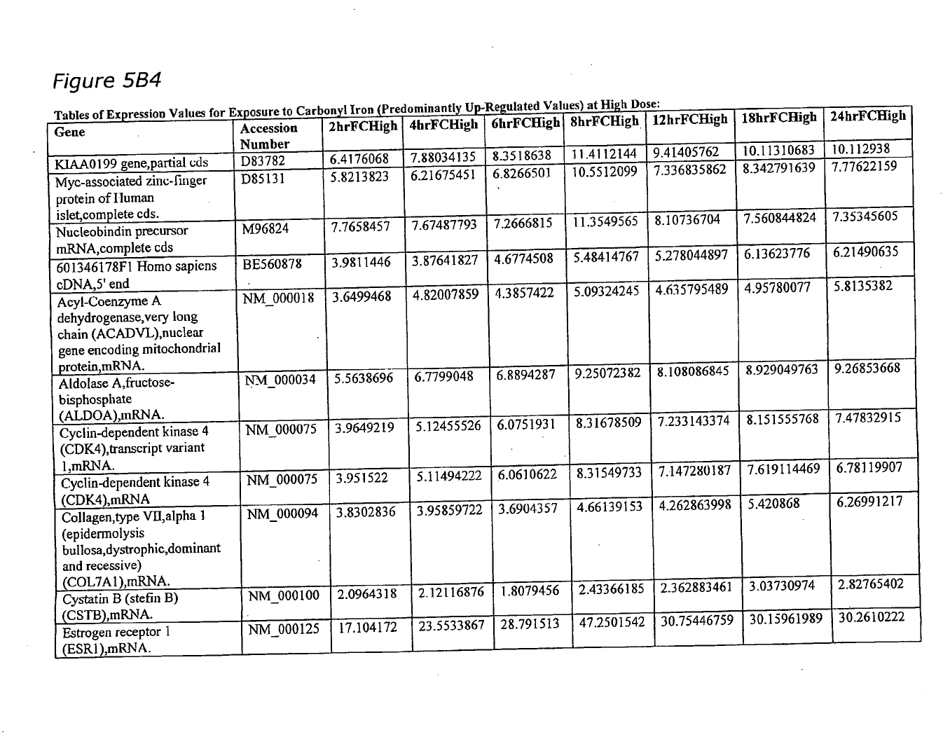

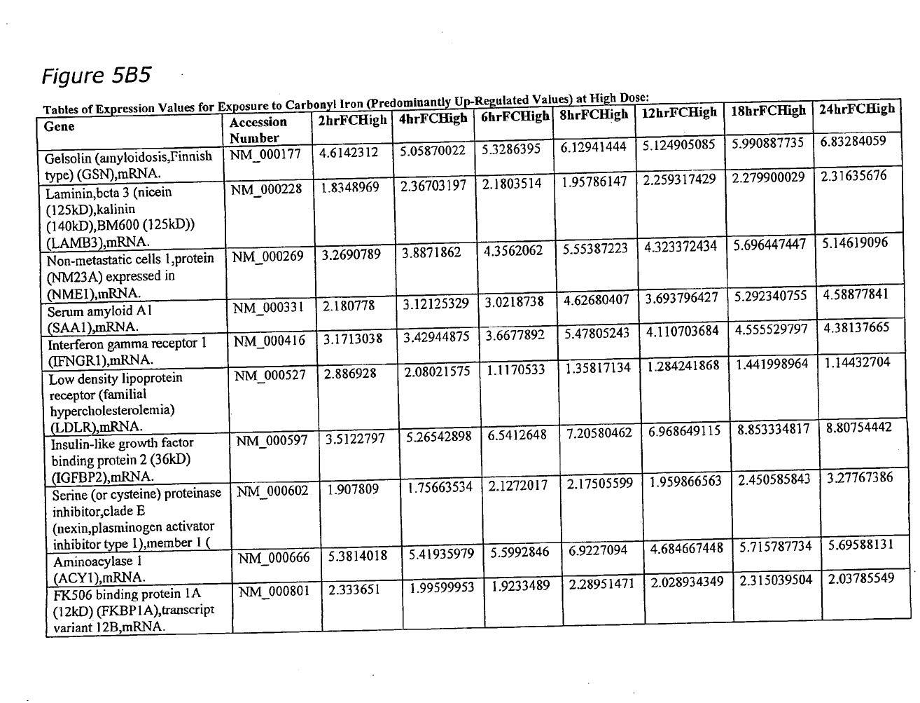

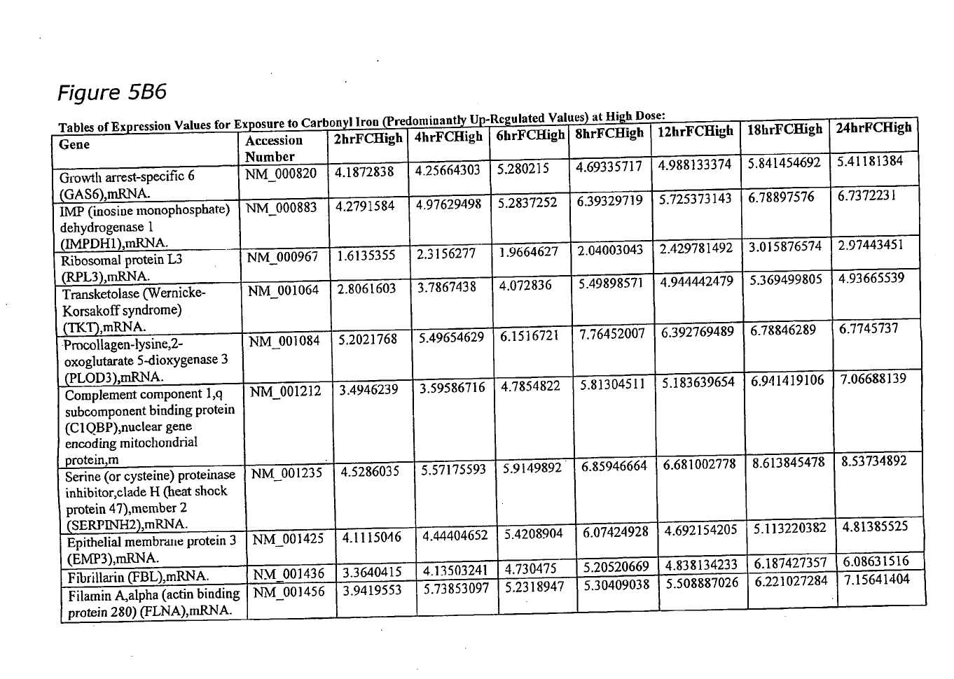

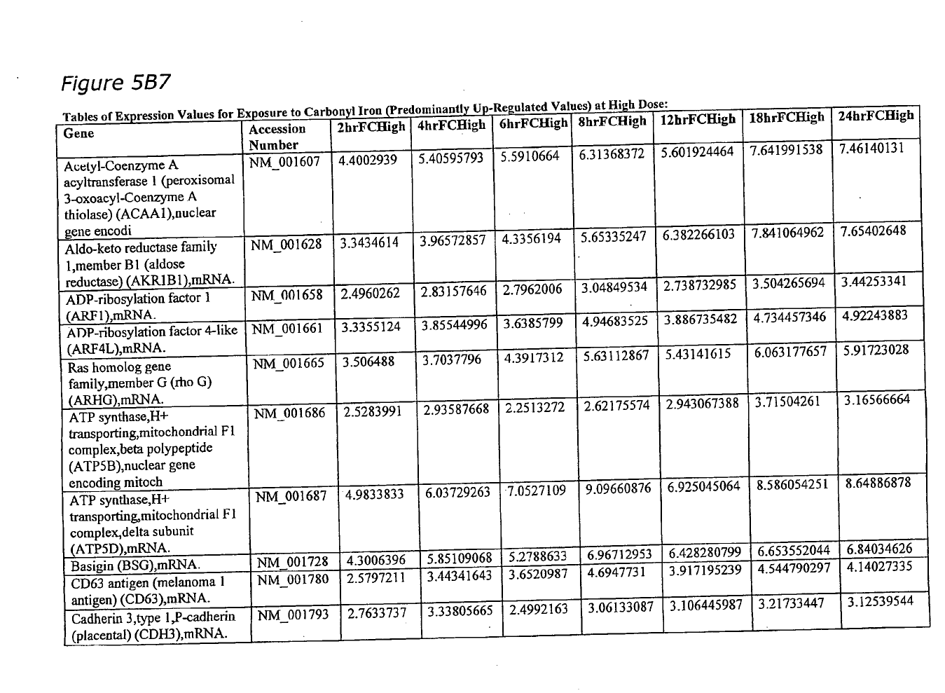

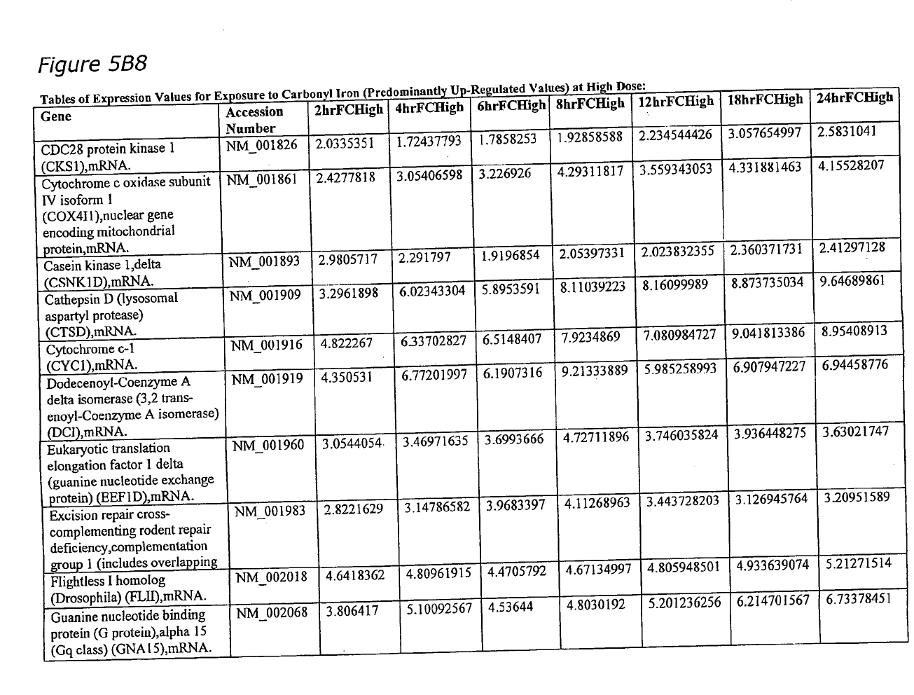

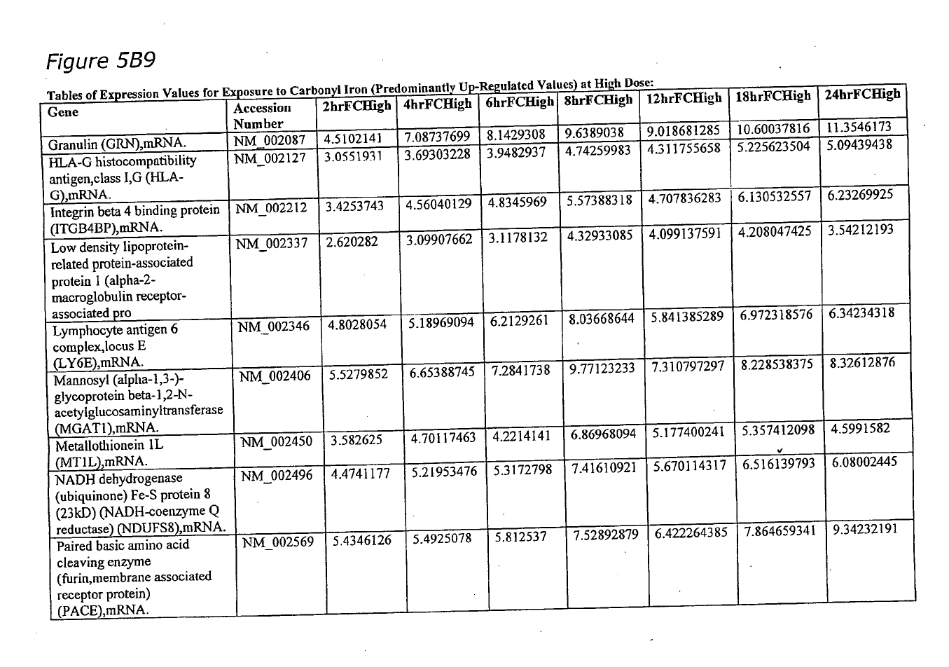

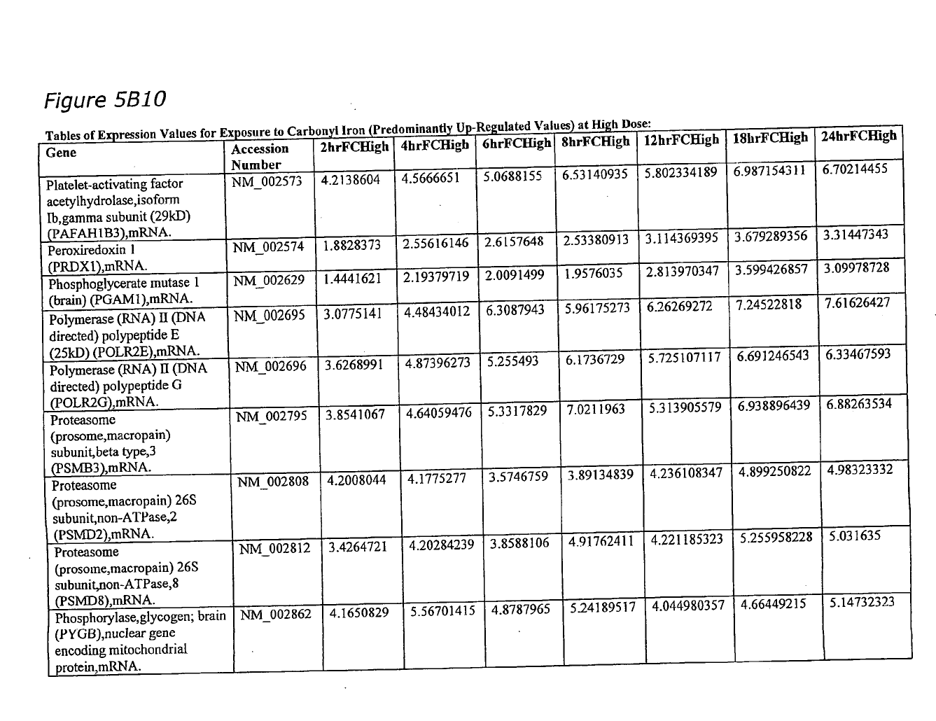

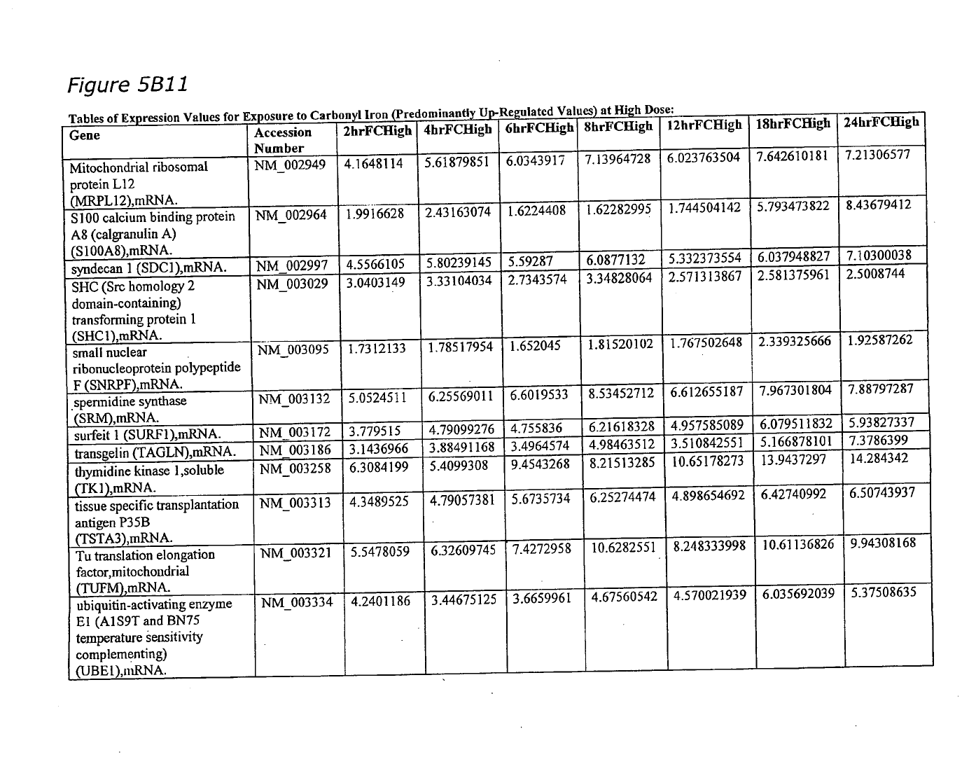

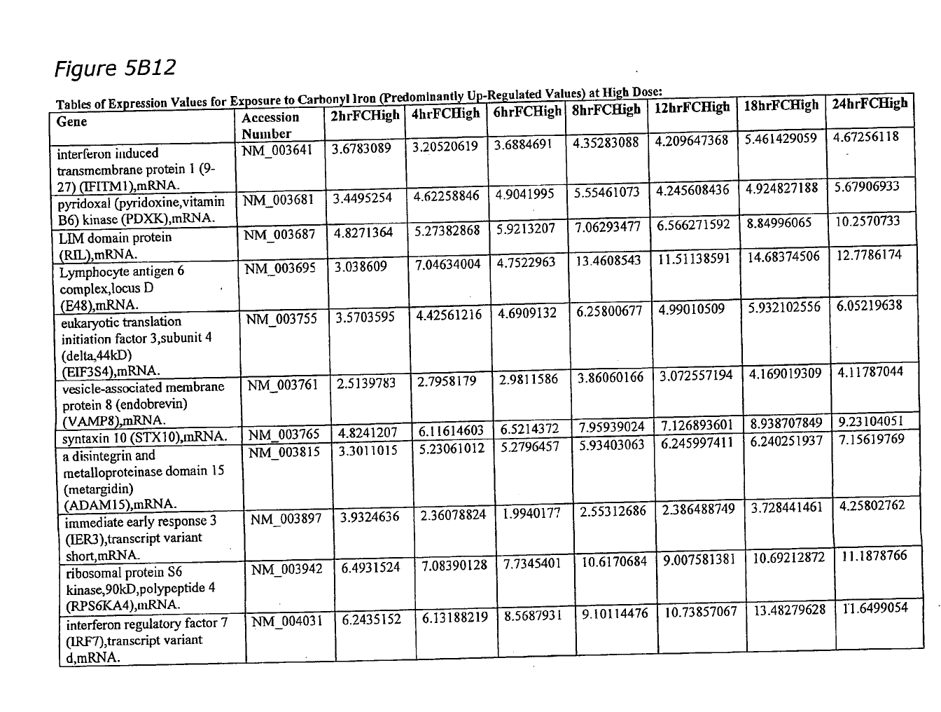

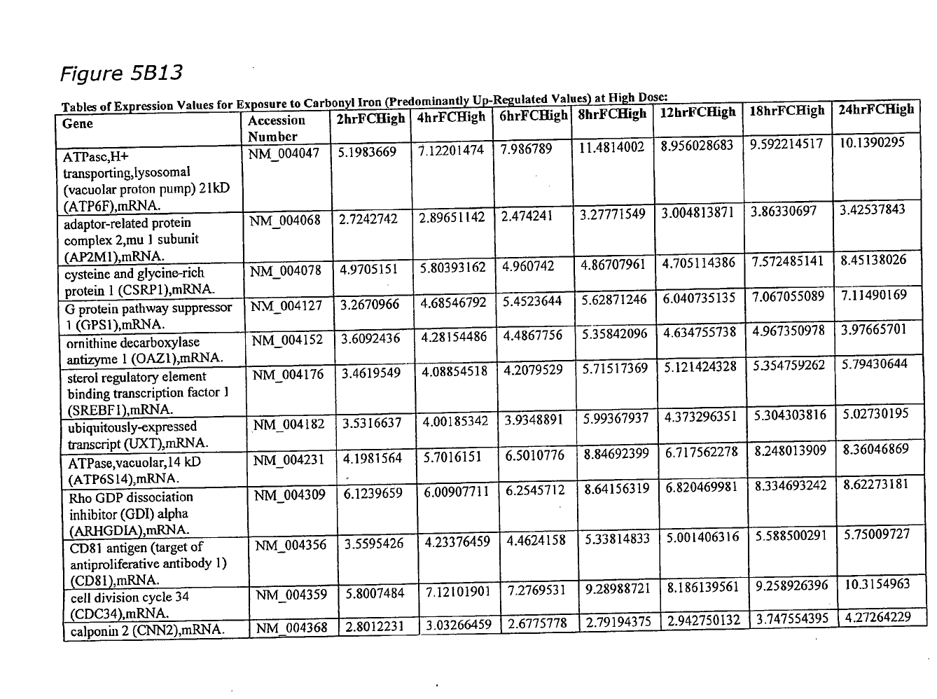

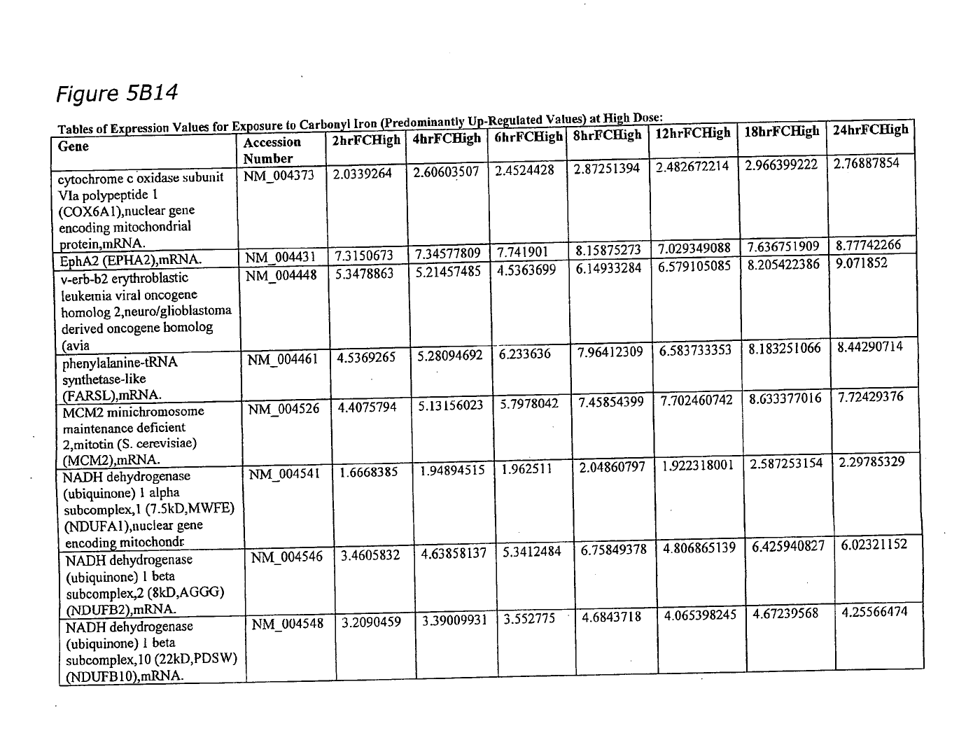

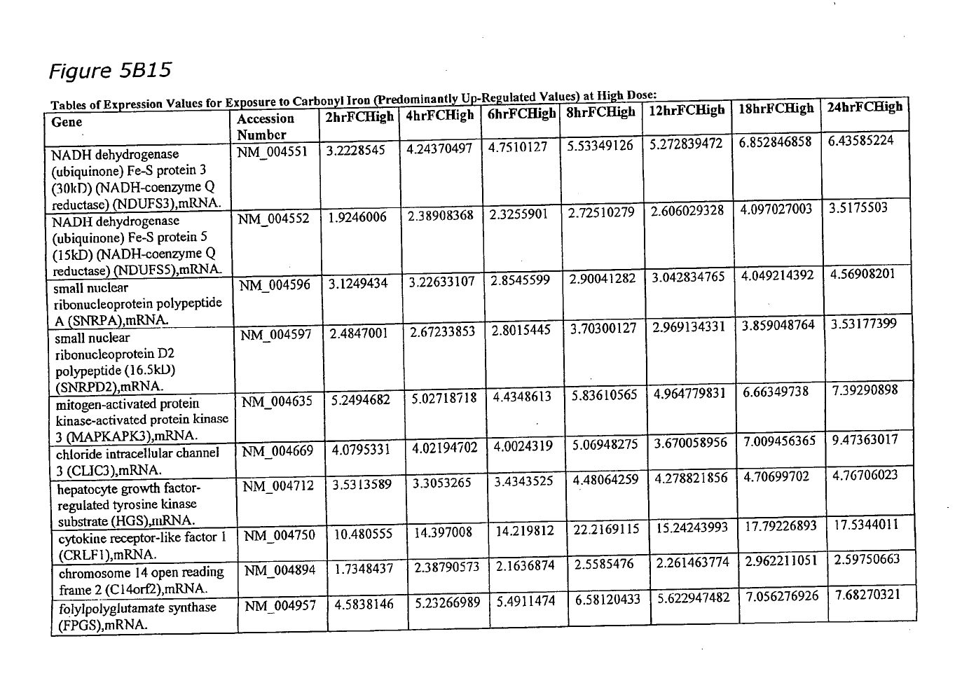

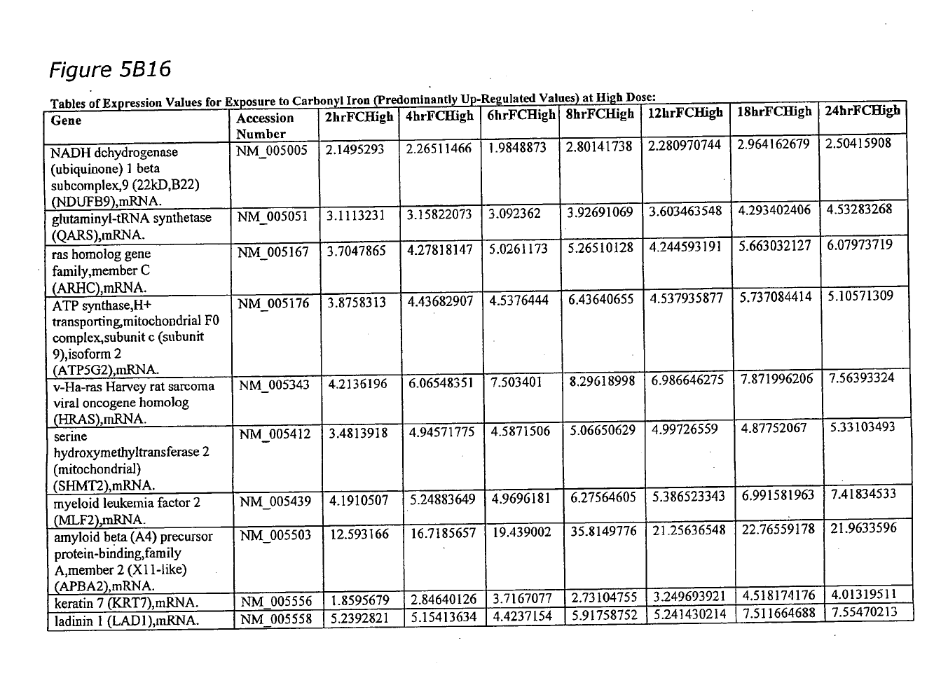

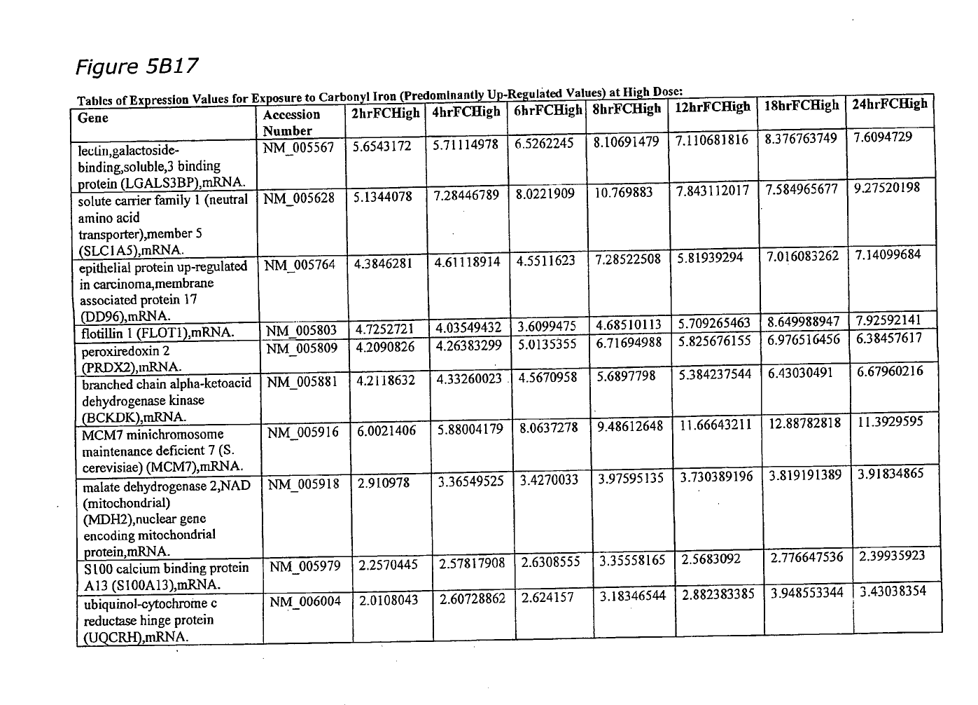

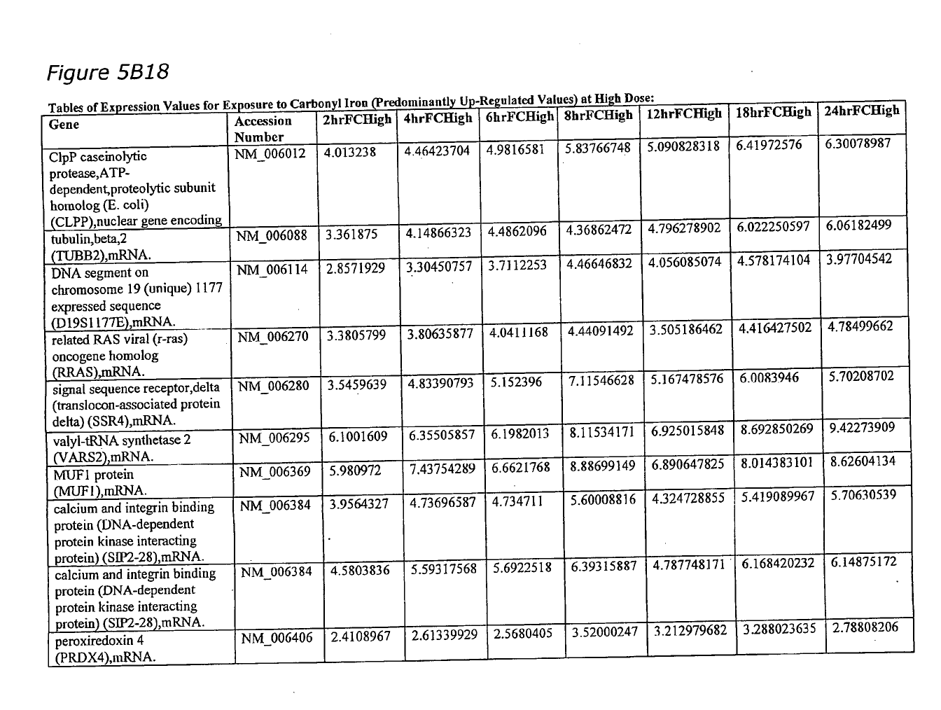

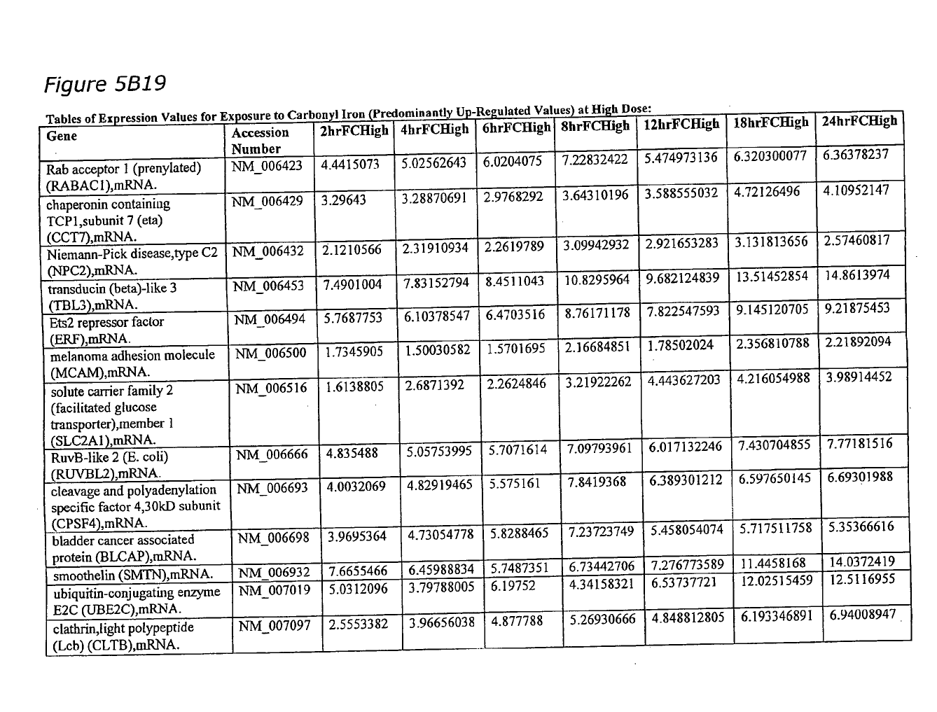

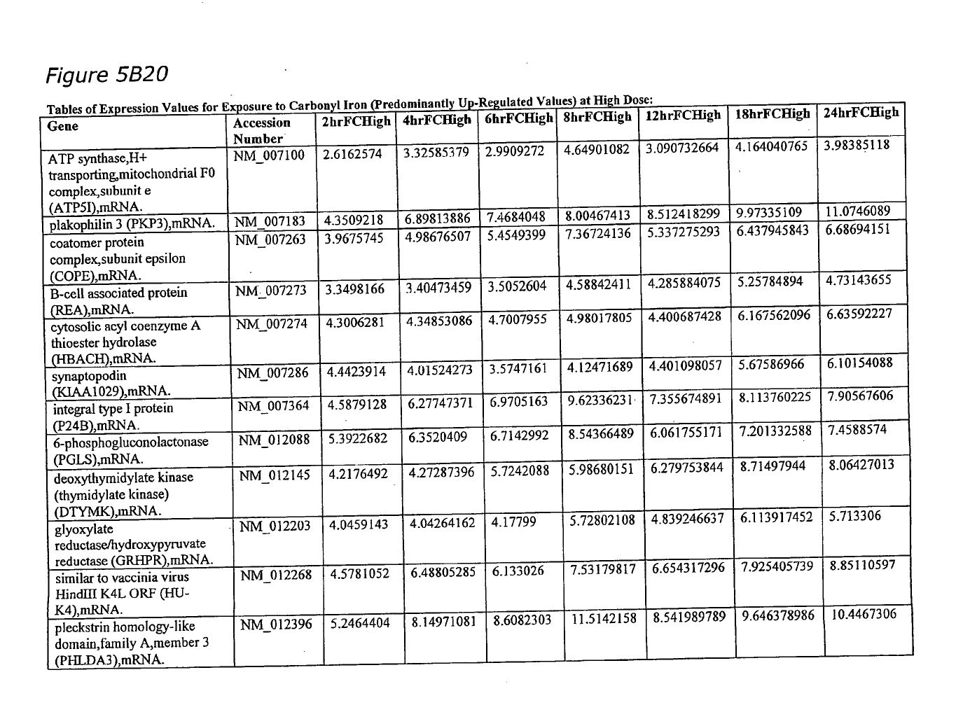

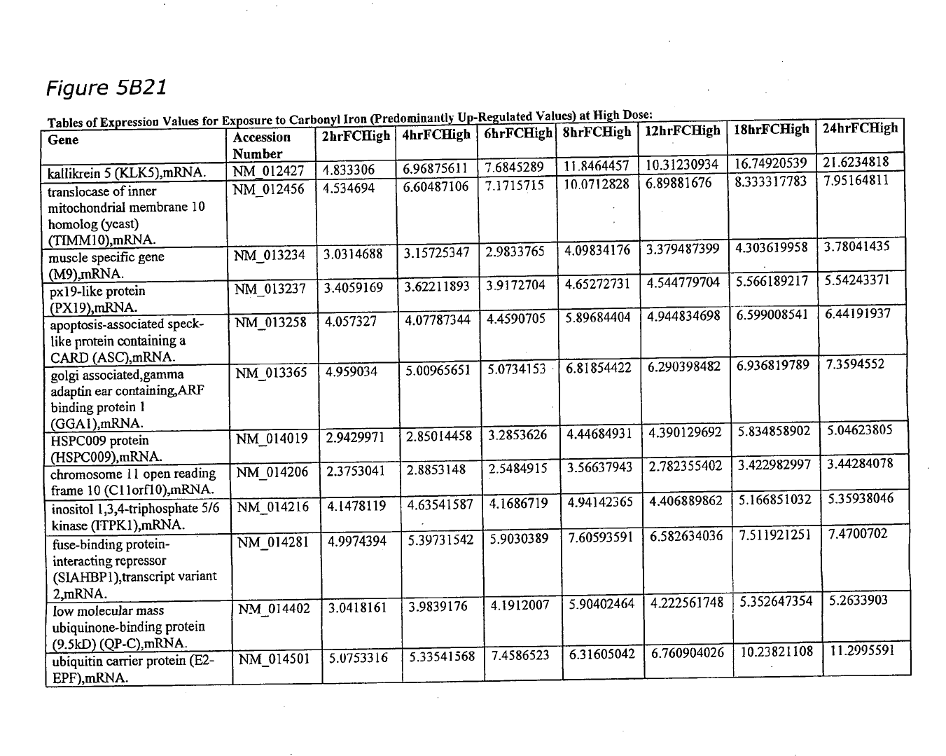

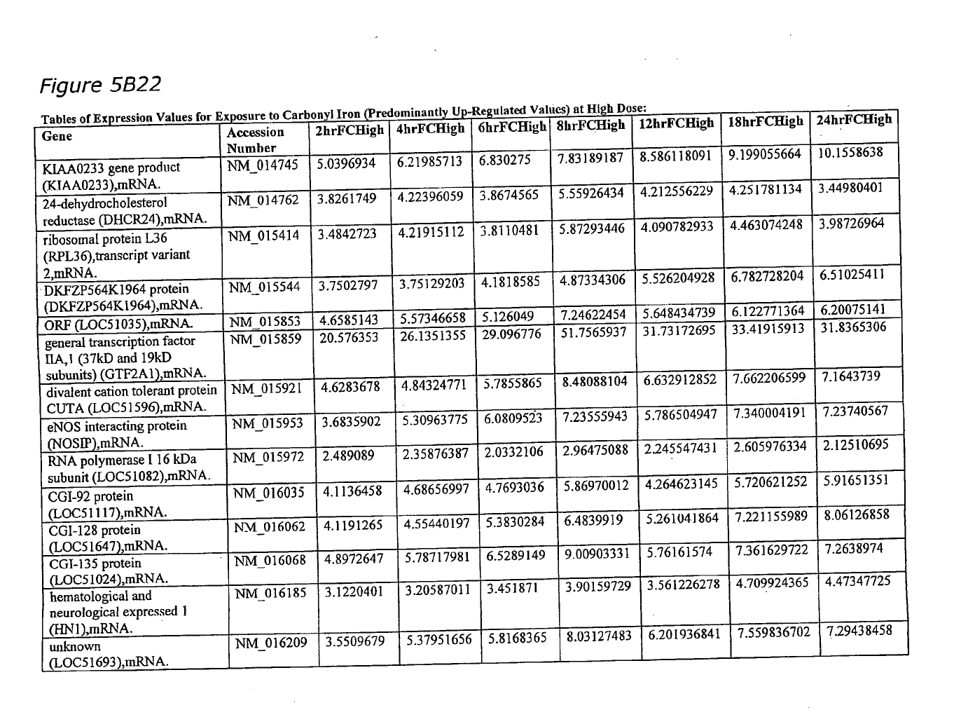

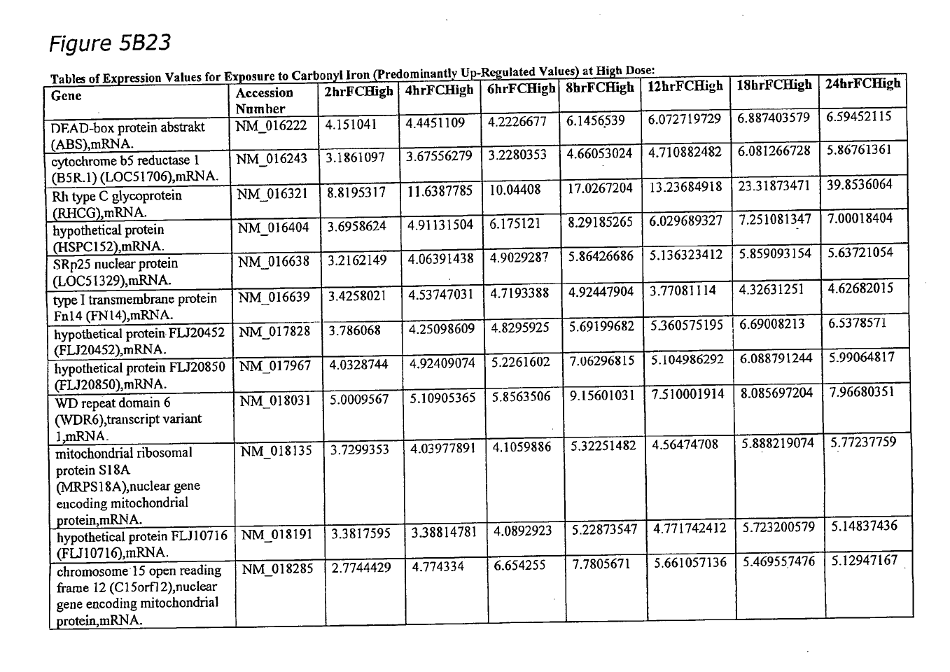

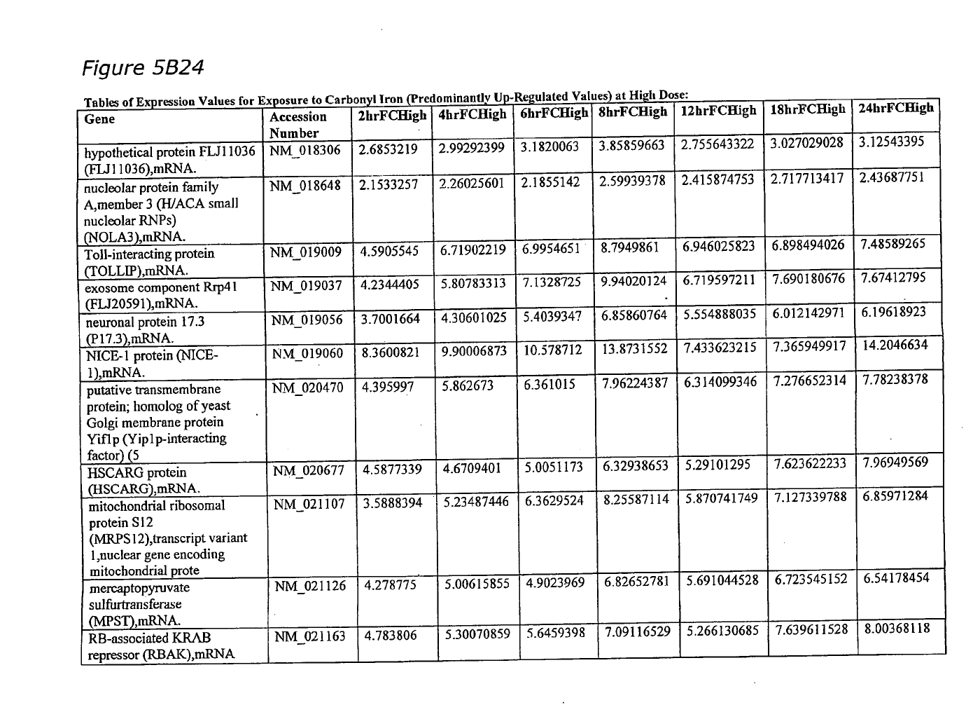

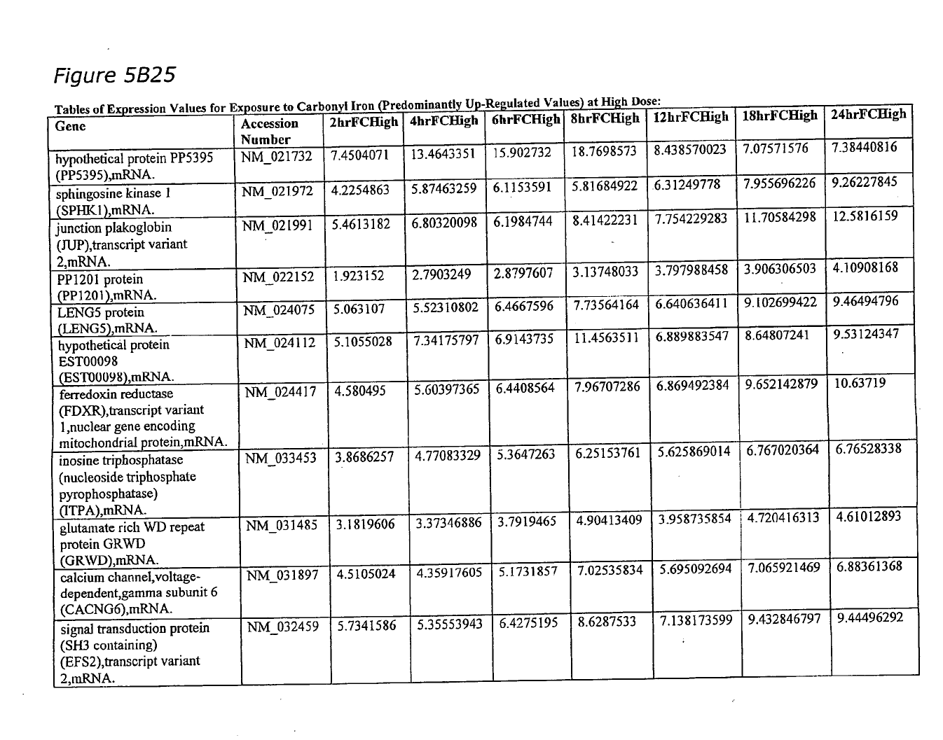

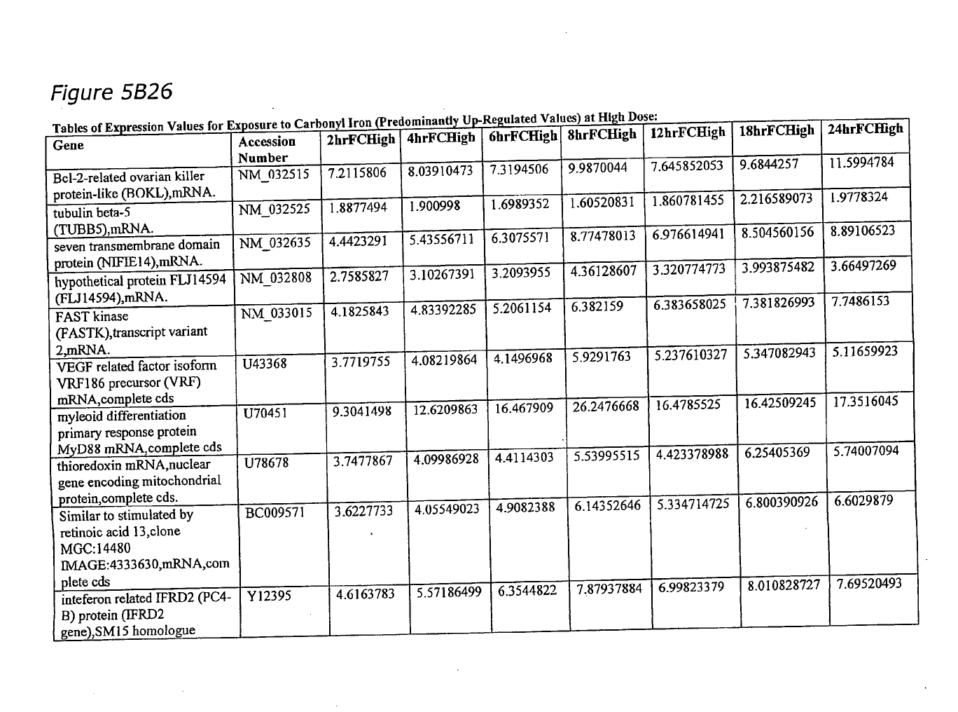

FIG. 5A and B present the genes primarily up-regulated by exposure to ferronyl iron, the data presented for the same genes at low and high dose and over time in HEK cells.

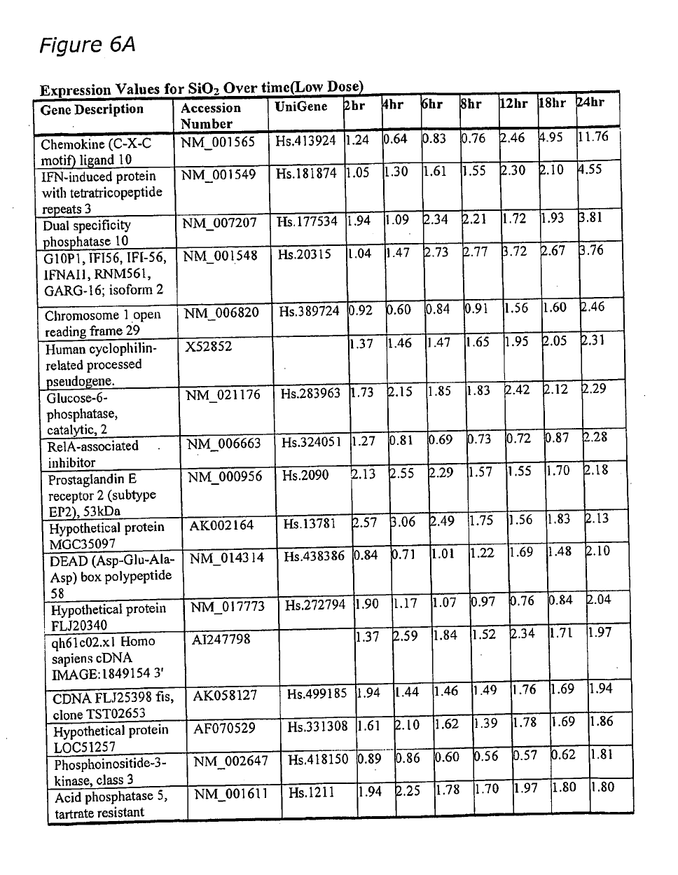

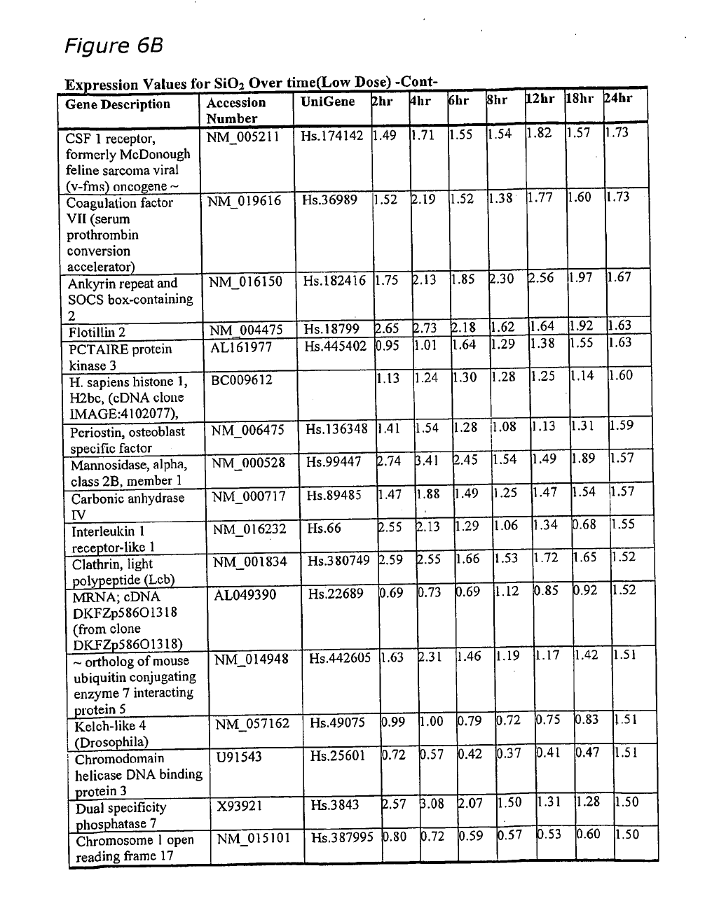

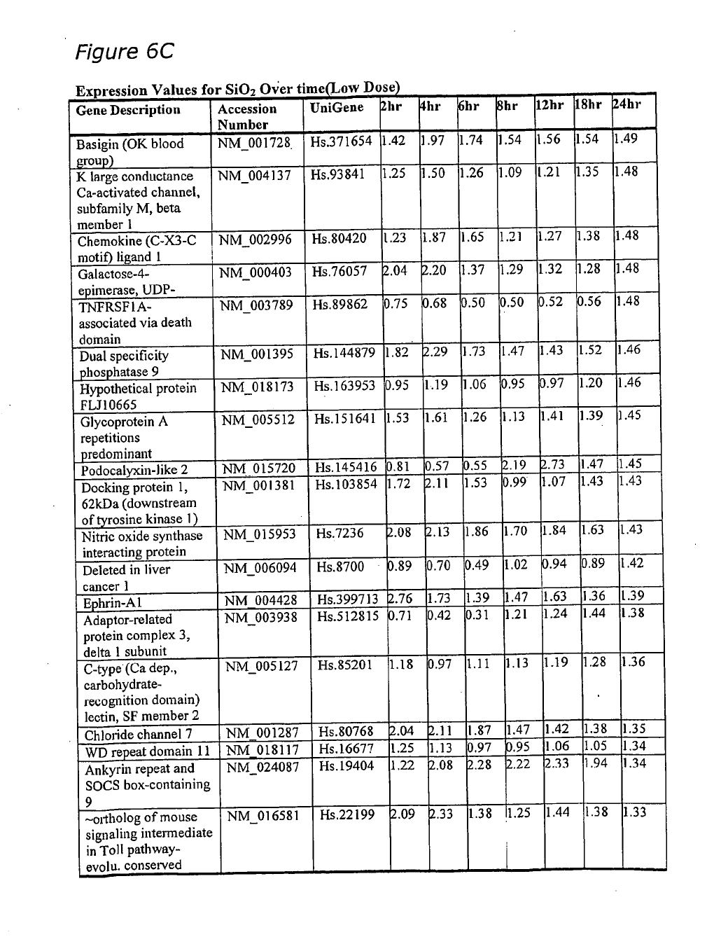

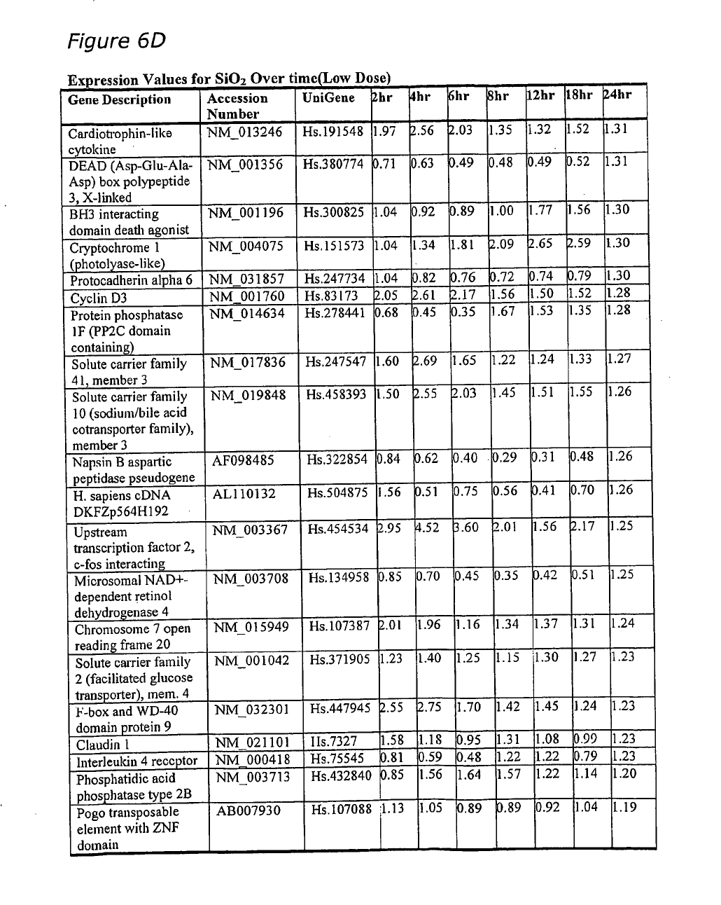

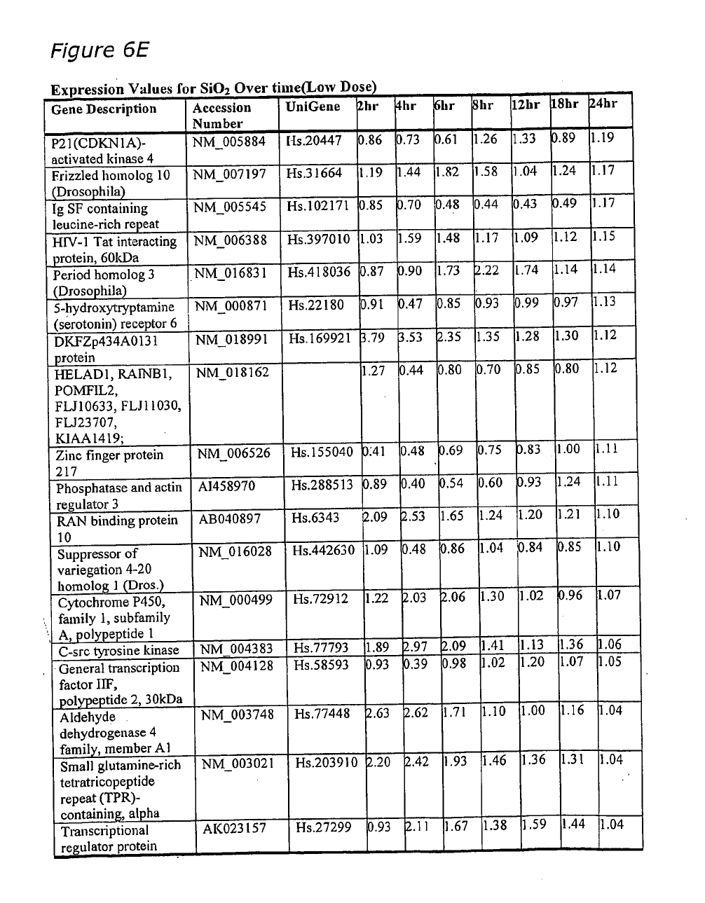

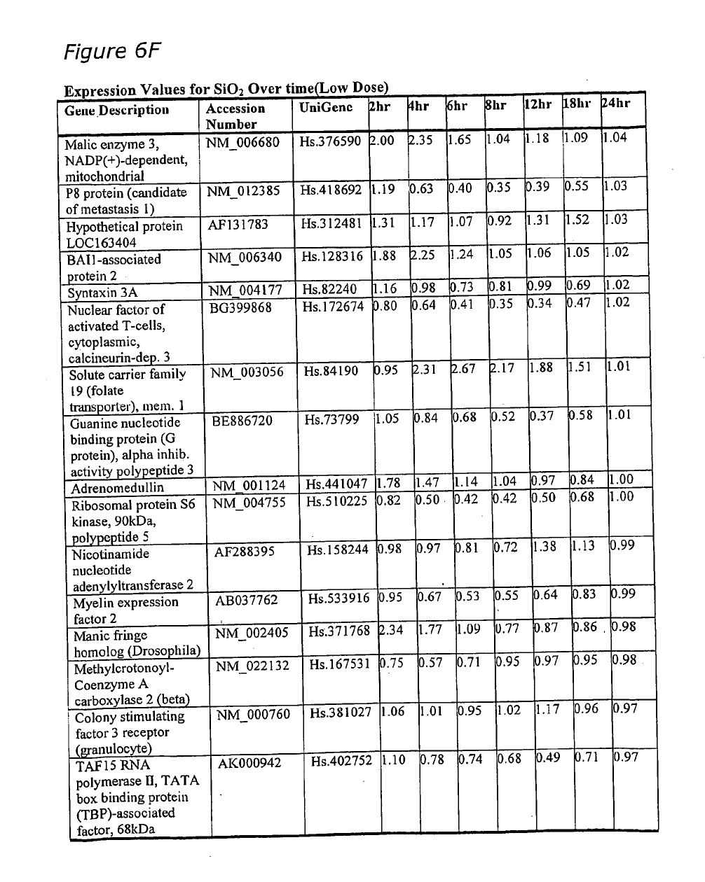

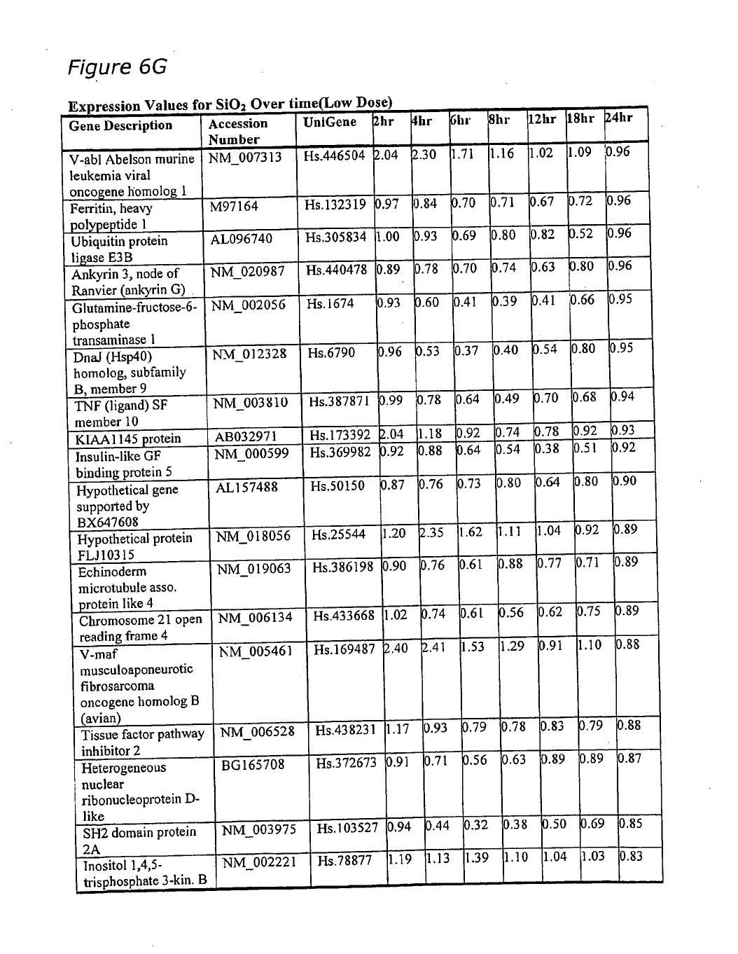

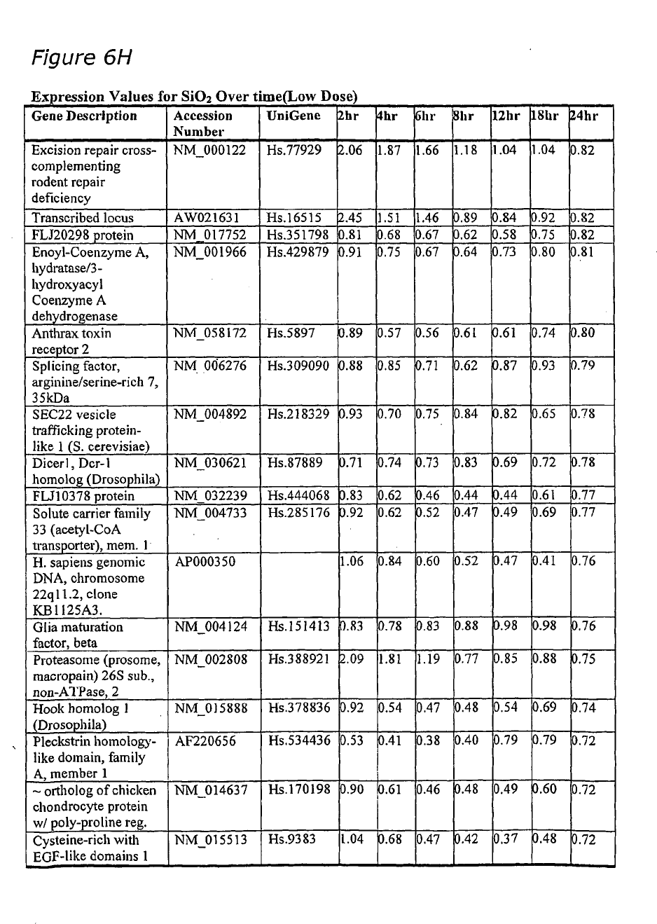

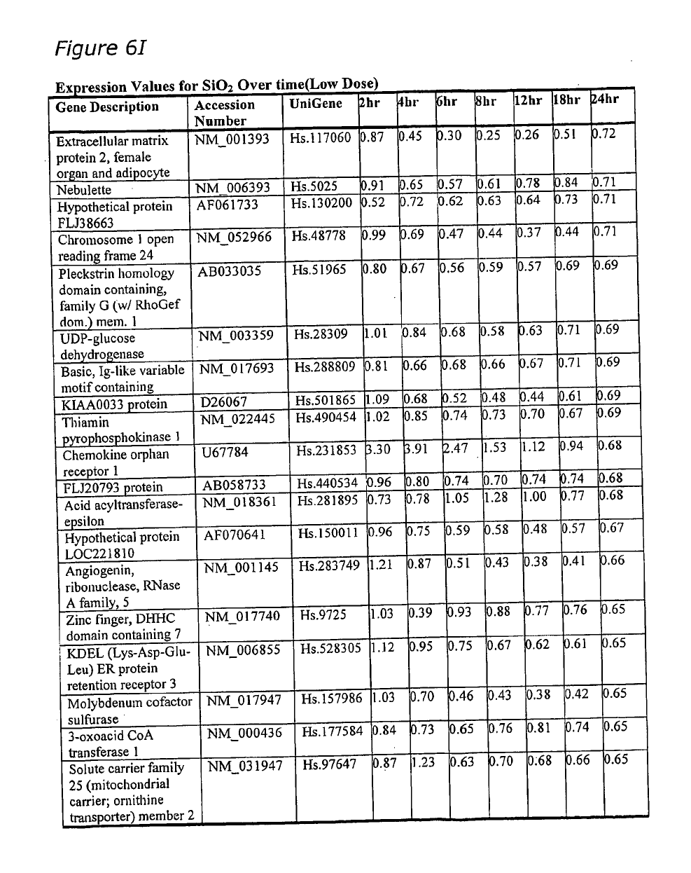

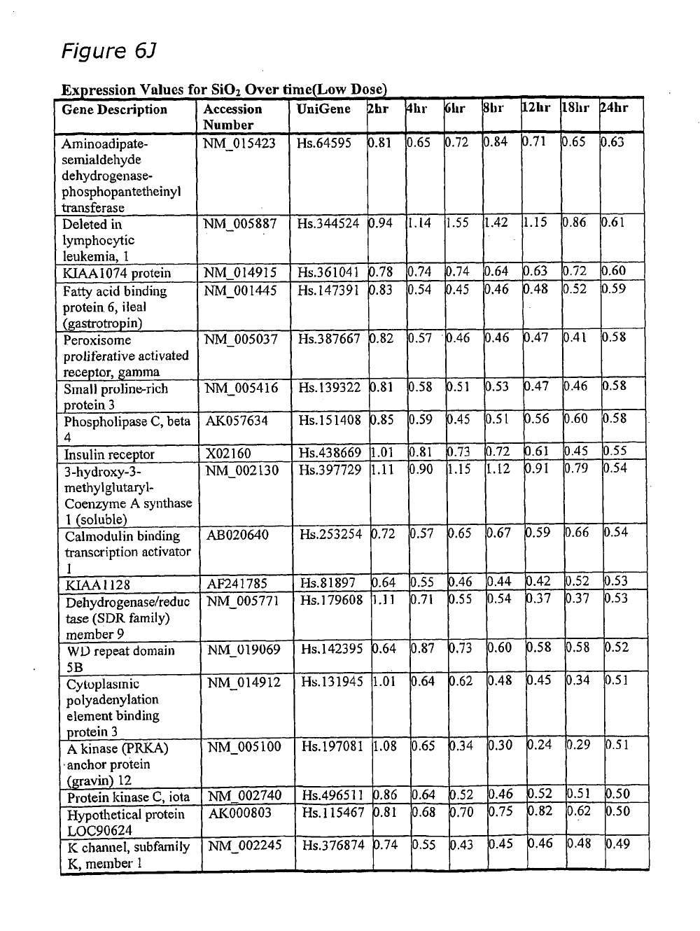

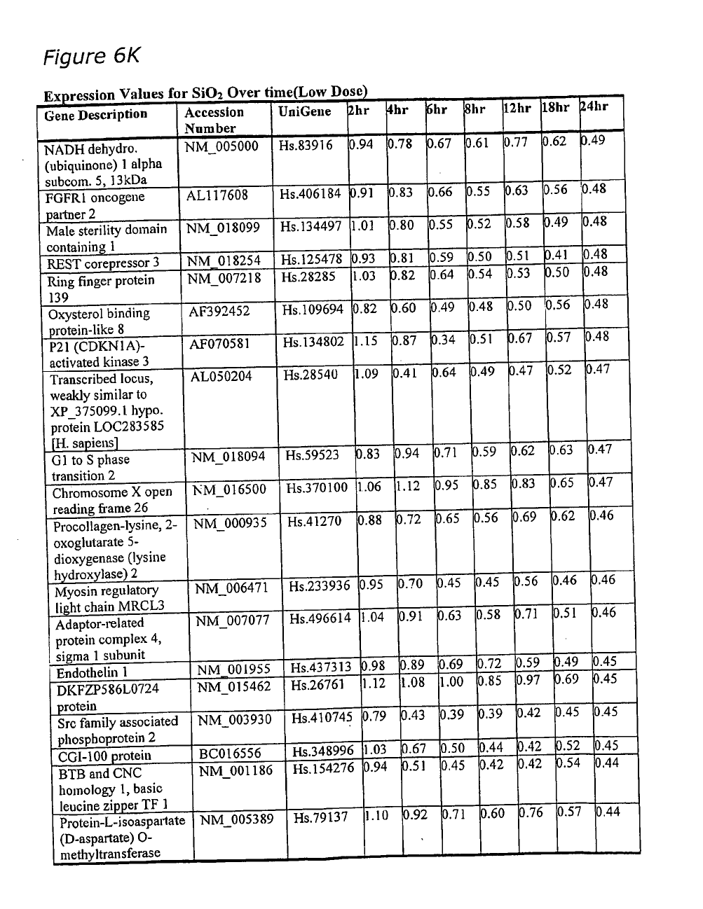

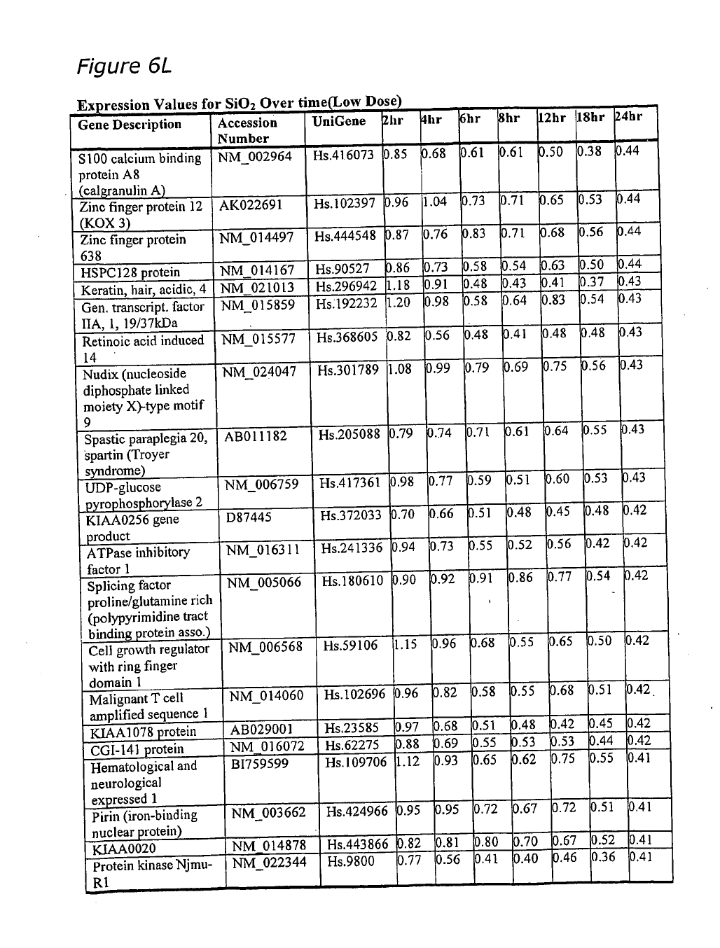

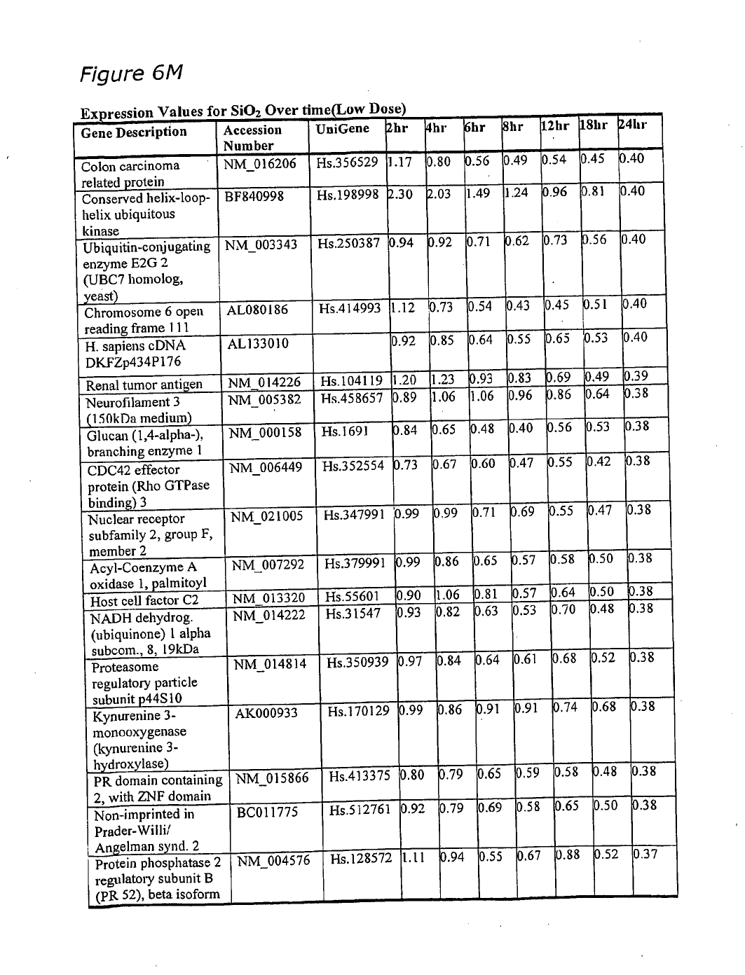

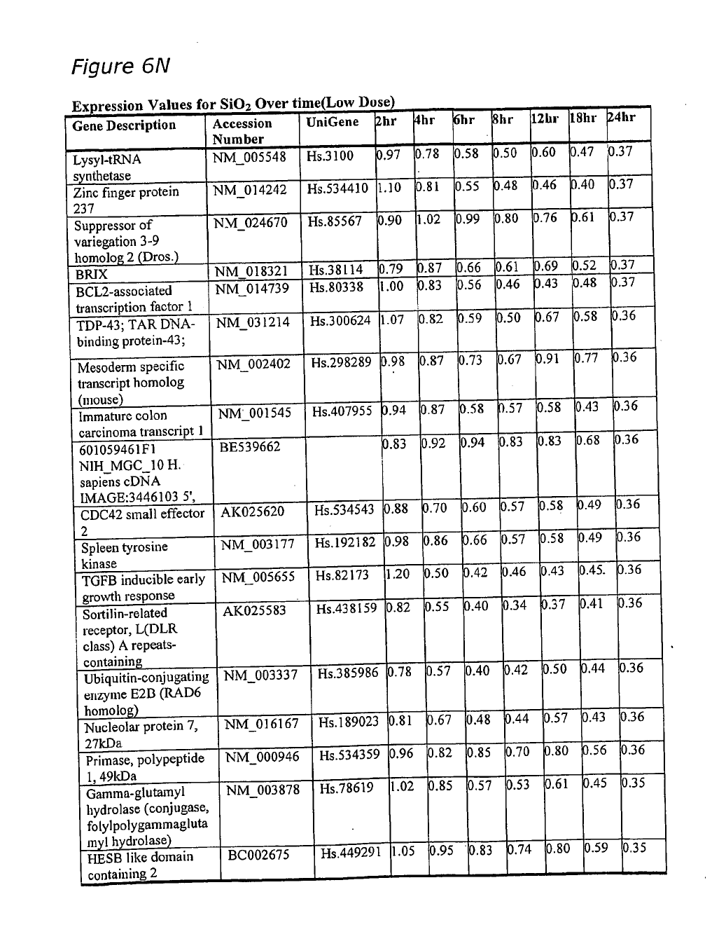

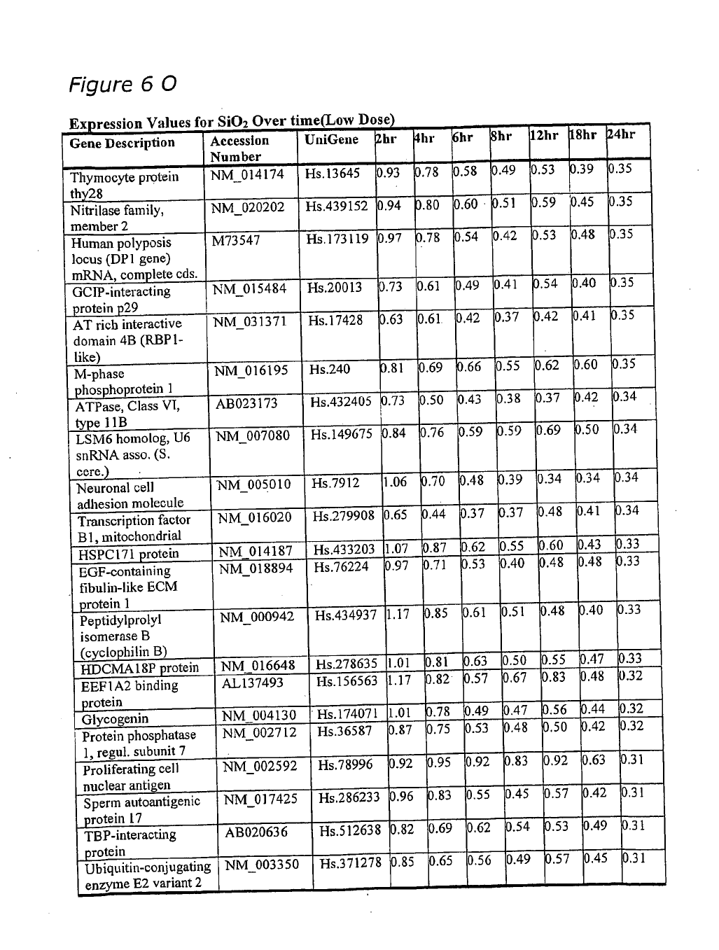

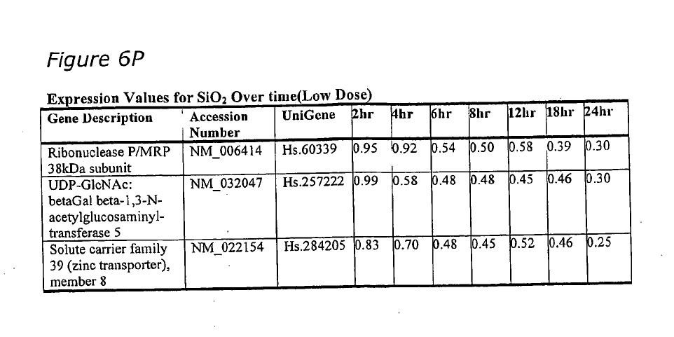

FIG. 6A-P presents GEM results for low dose SiO₂ nonoparticle exposure over time in HEK cells.

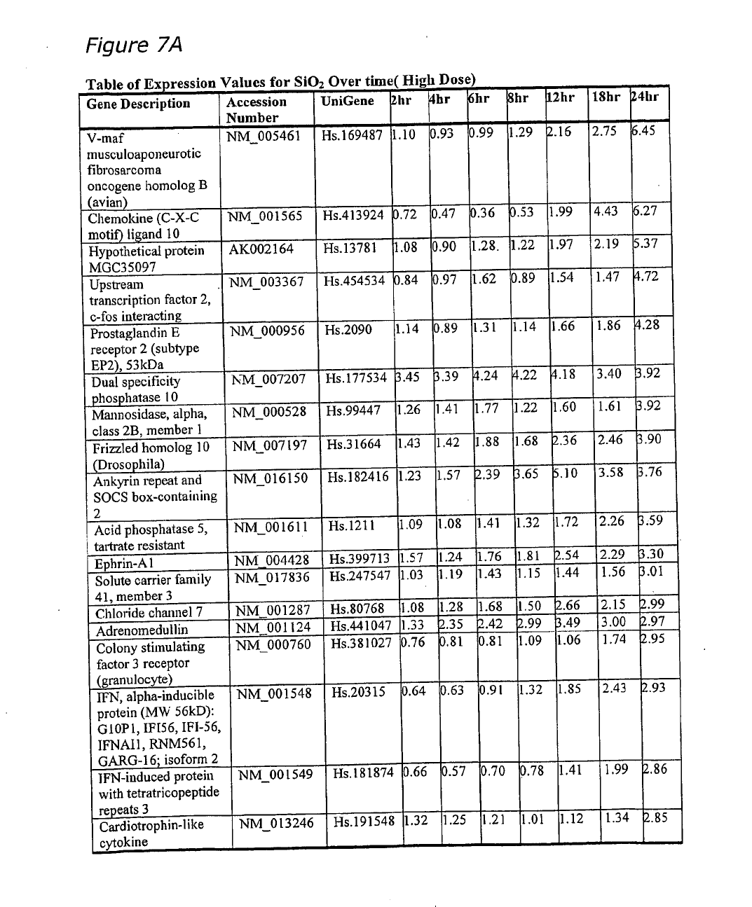

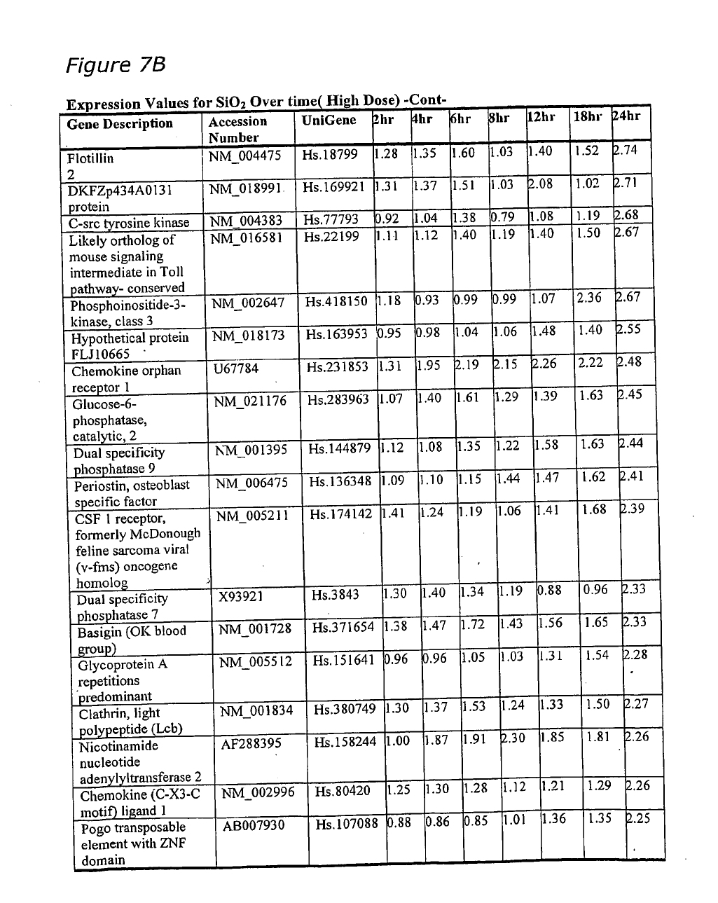

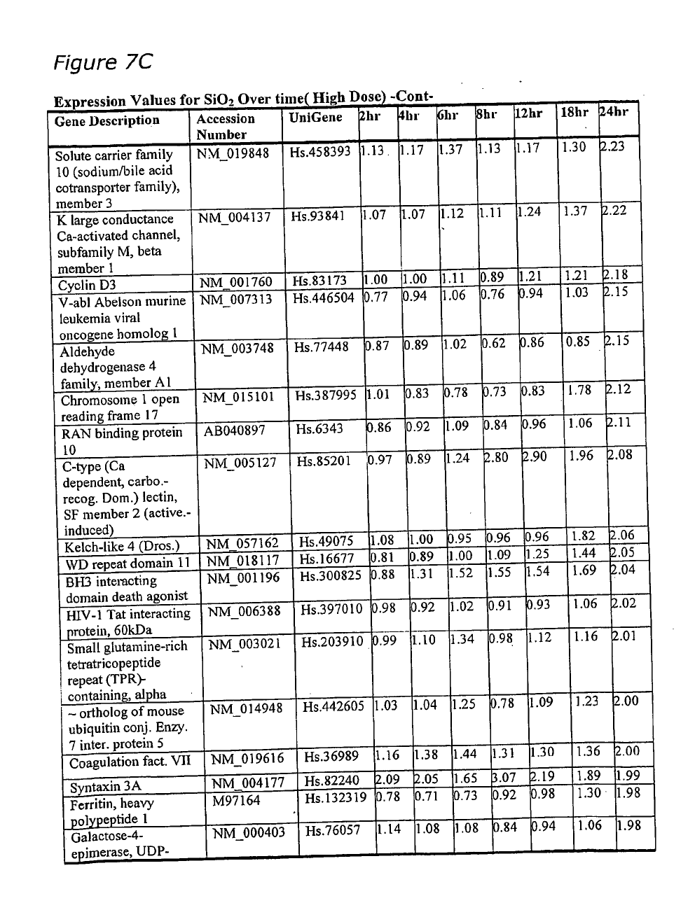

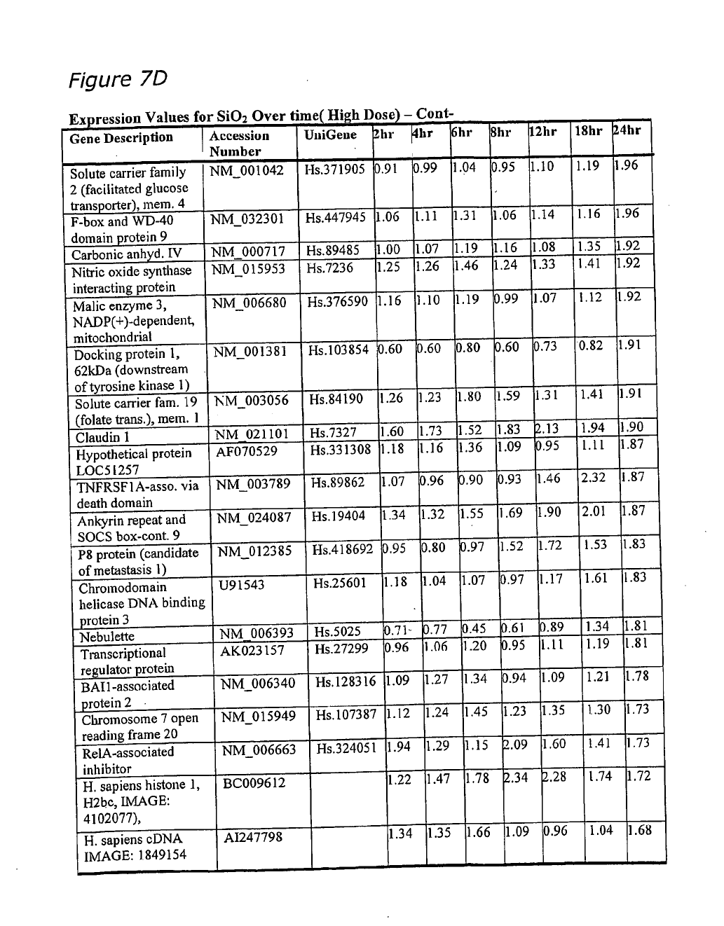

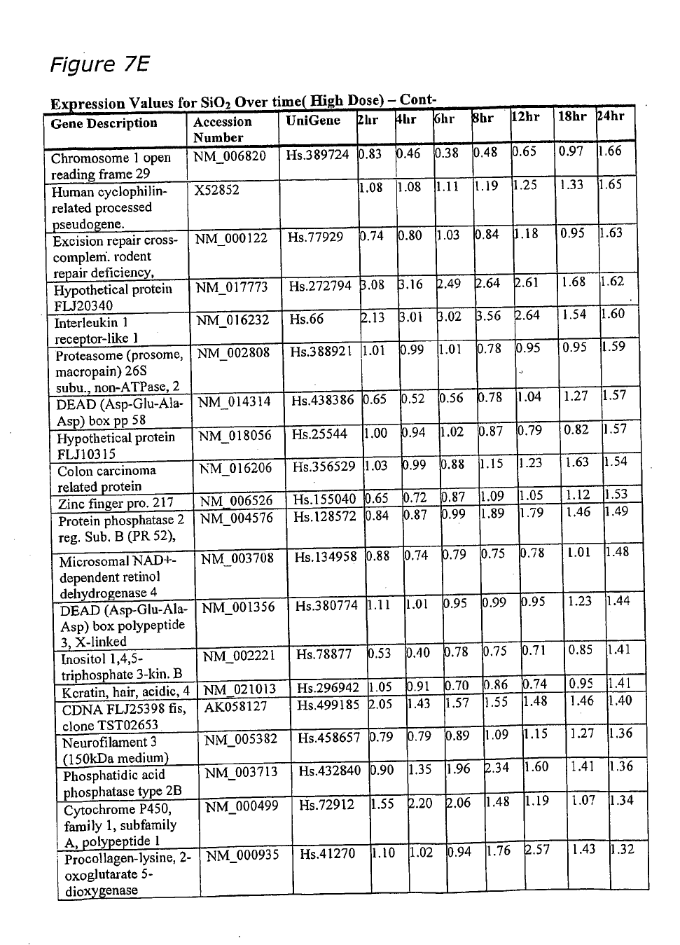

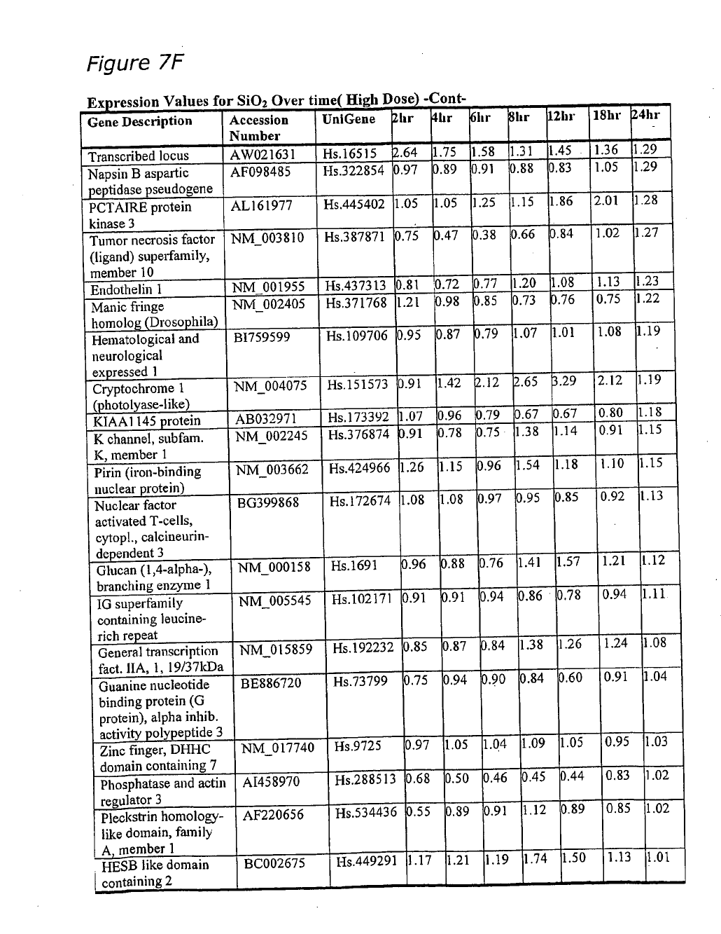

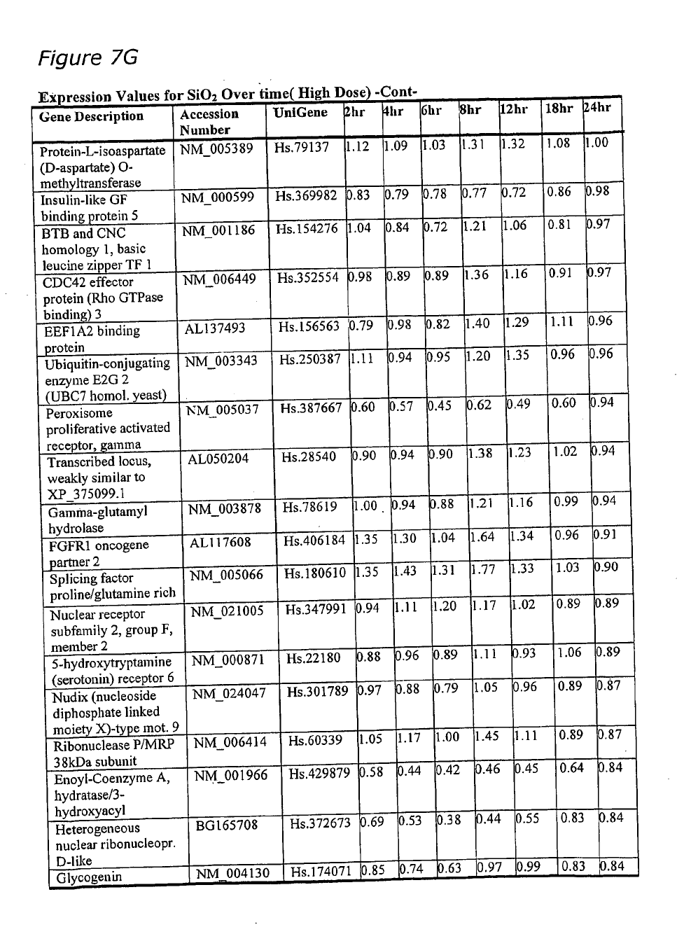

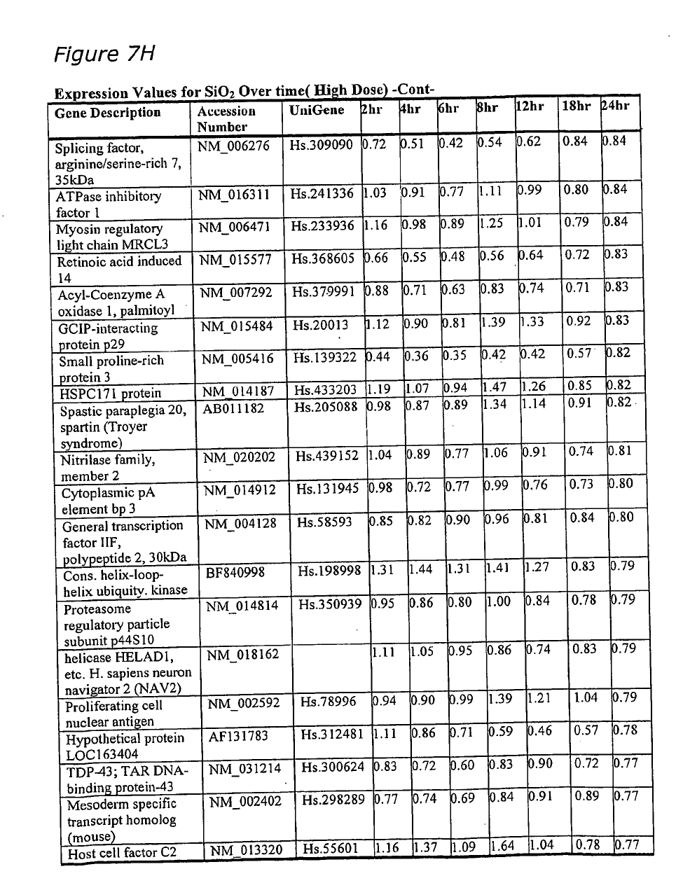

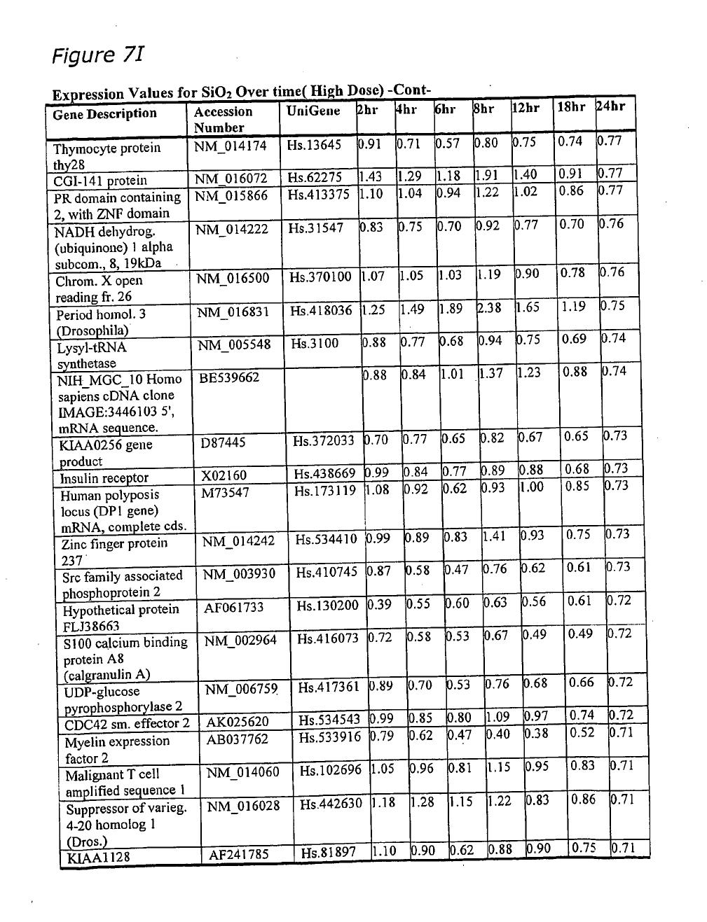

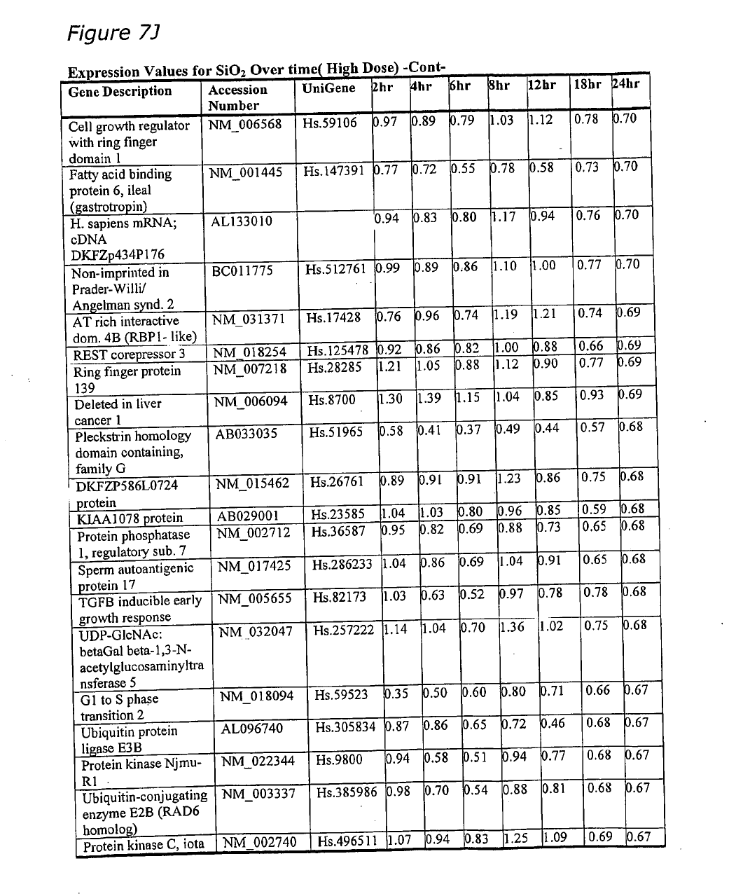

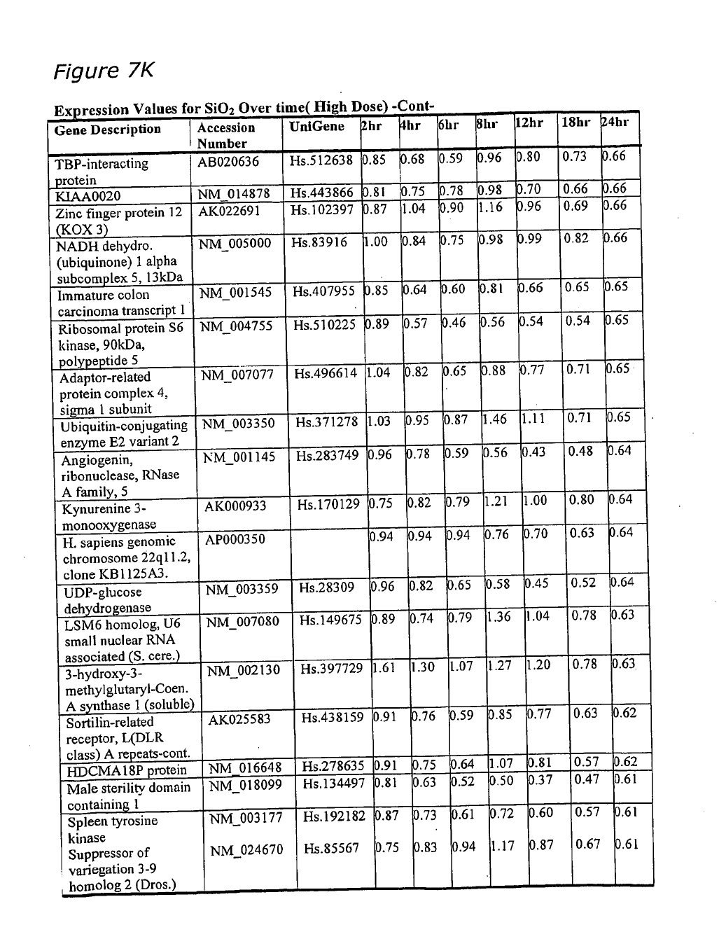

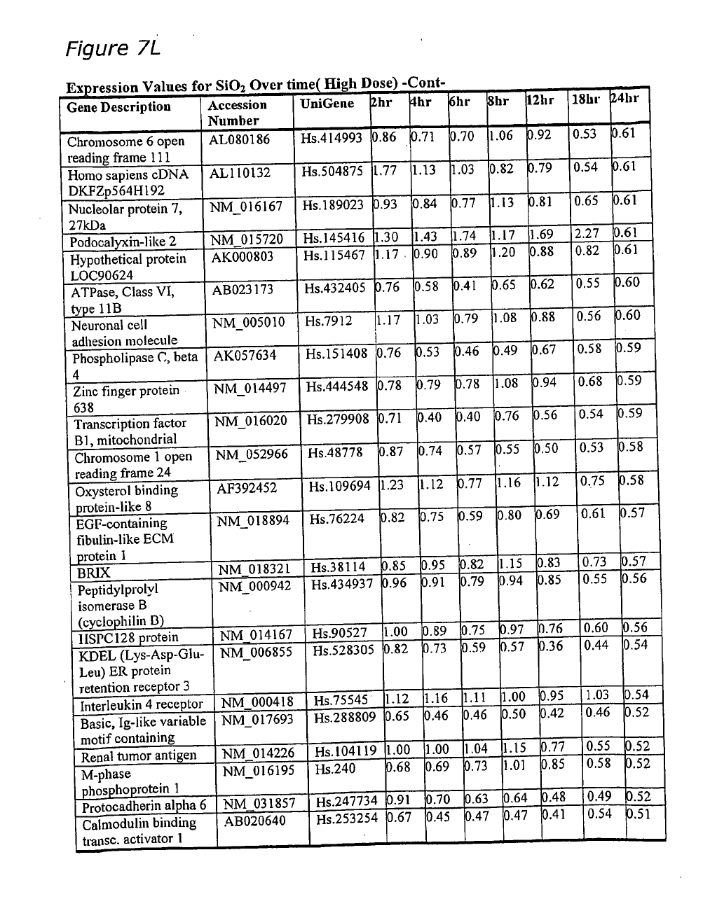

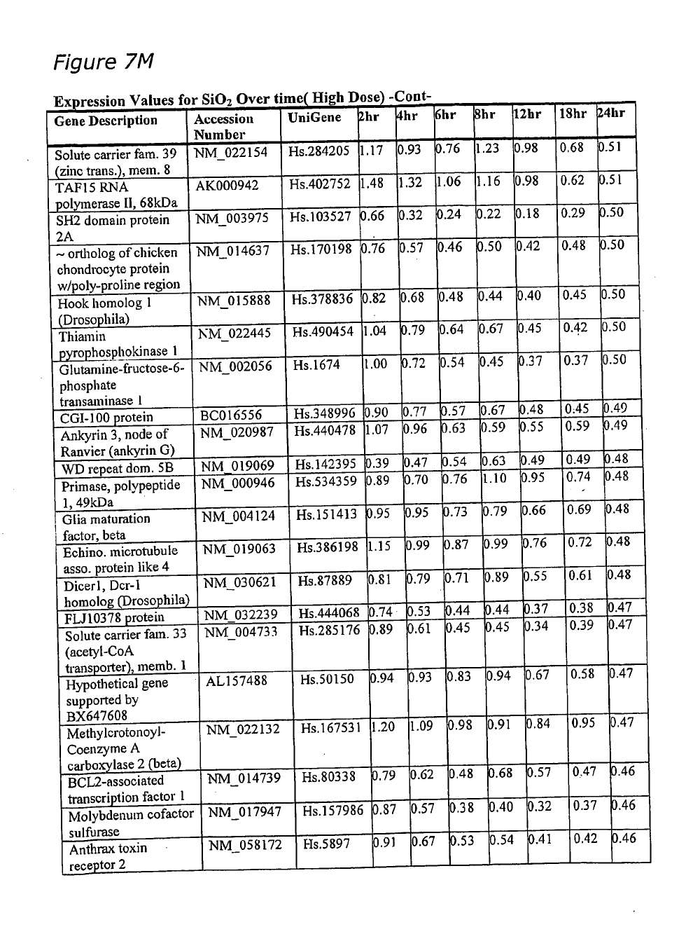

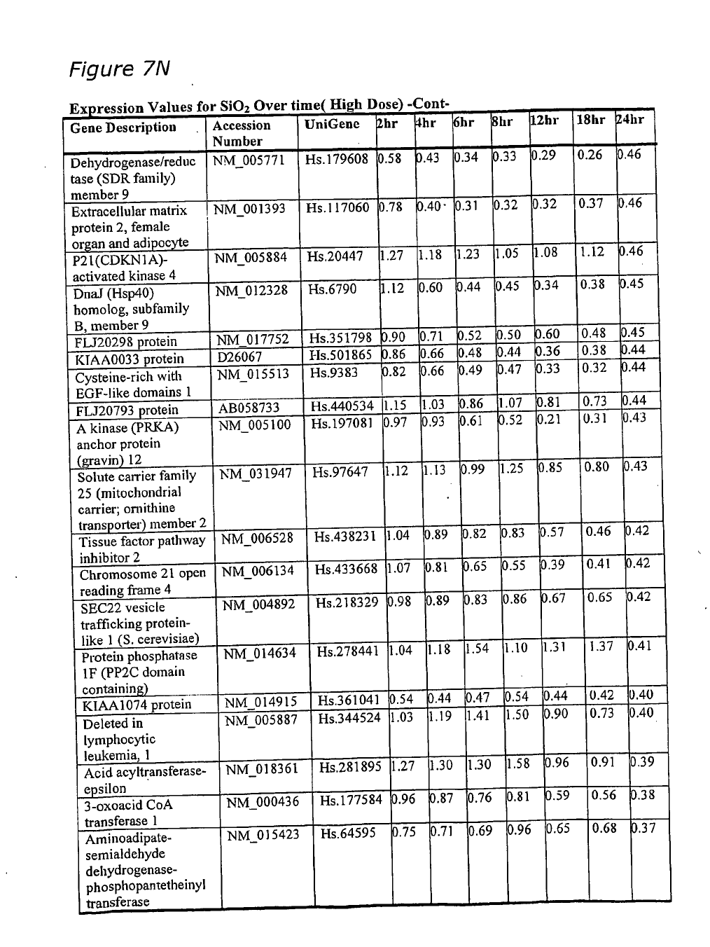

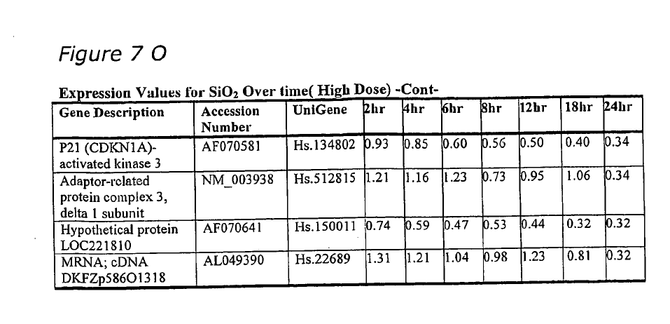

FIG. 7A-O presents GEM results for high dose SiO₂ nonoparticle exposure over time in HEK cells.

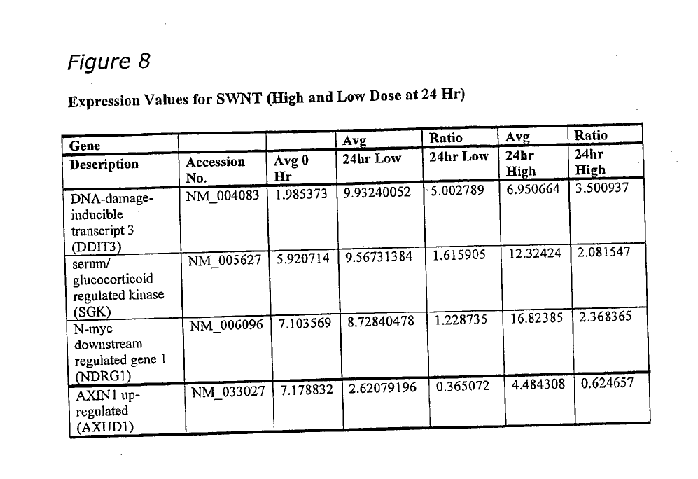

FIG. 8 presents GEM results for SWNT nanoparticle exposure at high and low doses at 24 hours in HEK cells. Upregulation of DNA-damage-inducible transcript 3 (DDIT3), serum/glucocorticoid regulated kinase (SGK), and N-myc downstream regulated gene 1 (NDRG1) was observed with SWNT exposure, while AXIN1 up-regulated (AXUD1) was down regulated.

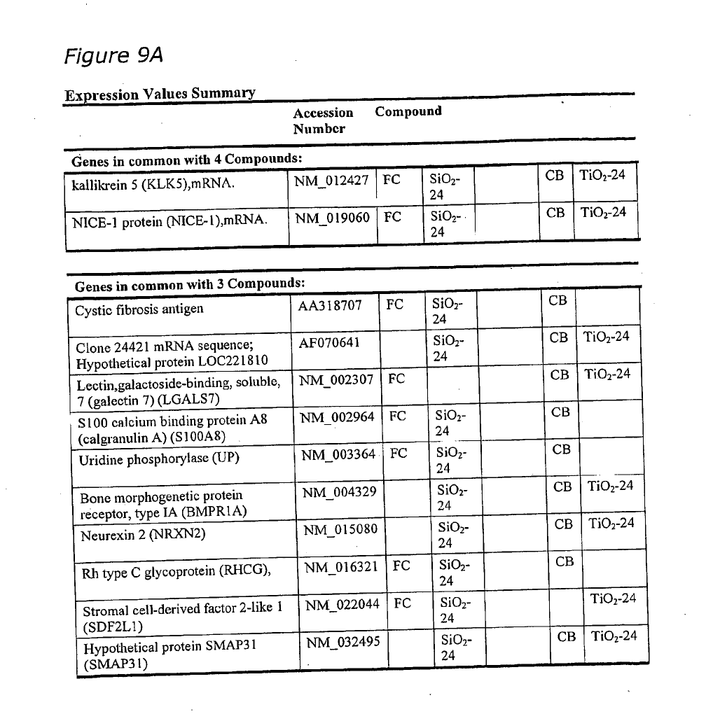

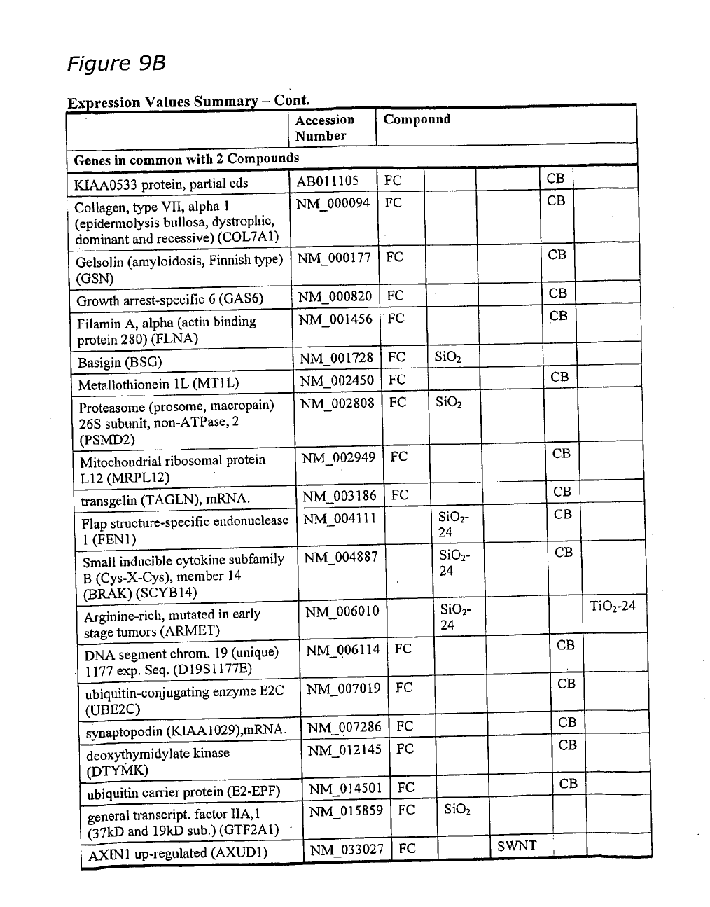

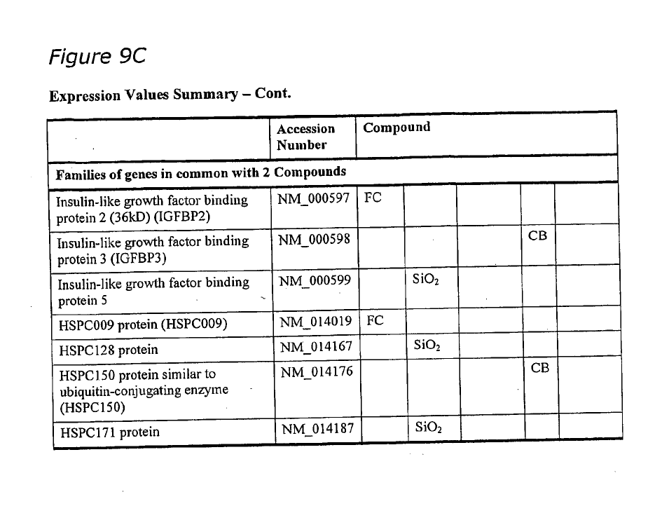

FIG. 9A-C presents summary results identifying biomarkers of nanoparticle exposure. As shown in FIG. 9A, Kallikrein 5 and Nice-1 were upregulated upon exposure to FC, SiO₂, CB, and TiO₂. The following biomarkers were differentially expressed upon exposure to 3 of 4 of FC, SiO₂, CB, and TiO₂: Cystic fibrosis antigen Clone 24421; Hypothetical protein LOC221810; (LGALS7); S100 calcium binding protein A8 (S100A8); Uridine phosphorylase (UP); Bone morphogenetic protein receptor type IA (BMPR1A); Neurexin 2 (NRXN2); Rh type C glycoprotein (RHCG); Stromal cell-derived factor 2-like 1 (SDF2L1); Hypothetical protein SMAP31 (SMAP31); DNA-damage-inducible transcript 3 (DDIT3); serurm/glucocorticoid regulated kinase (SGK); N-myc downstream regulated gene 1 (NDRG1); AXIN1 up-regulated (AXUD1); and combinations thereof. FIG. 9B-C presents biomarkers that were differentially expressed upon exposure to 2 of 5 of FC, SiO₂, CB, TiO₂, and SWNT.

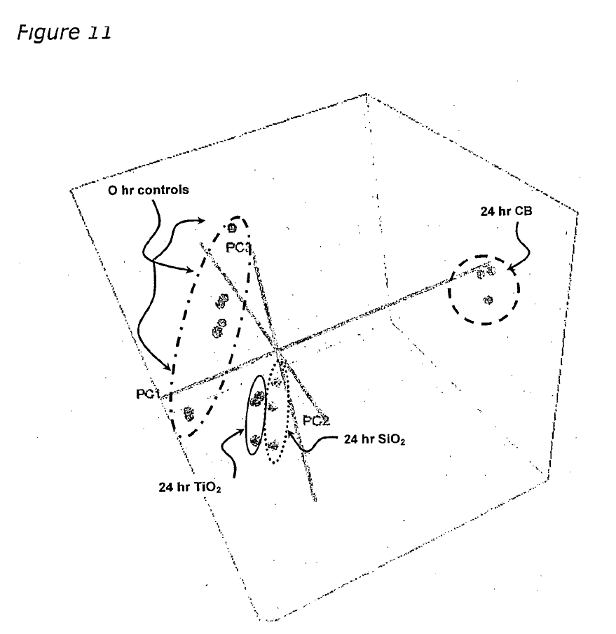

FIG. 11 graphically depicts principal components analysis for nanomaterial exposure and depicts a visual method for identification of nanoparticle exposure by cells, comprising comparing GEM profiles from exposed or putatively exposed cells with GEM profiles from control cells by three dimensional display of principal component analysis data.

### EXAMPLE IV

### GEM Testing with Relaxed Stringency, including further Time Course Experiments

Elimination of candidate markers on the basis of any low quality flags may result in loss of important markers from the results. Thus, the above experiements were repeated with relaxed stringency as to the elimination of markers. Details on the analysis used for full time course data of Carbon Black (CB), Carbonyl Iron (FC), Silica (SiO2), and SWNT were as follows: 1) the data used for the analysis was categorized as High Dose (HD) and Low Dose (LD); 2) normalized intensities (gene expression values) for all microarray probes annotated as “Discovery” (non control probes) and with a quality flag of “good” (fluorescent signal for the probe spot on the array conformed to specifications, was not contaminated, irregular or low intensity) are used; 3) gene expression values are from three microarrays run on the same biological sample (triplicates) at 8 different time points—0, 2, 4, 6, 8, 12, 18 and 24 hours; and 4) MIAME guidelines were followed for all experiments on gene microarrays.

Materials used: The nano materials shown in bold below were used in the experiments

| Sample Name | Trade Name | Size | Mfg | Cat. No. | CAS No. | FW |
|---|---|---|---|---|---|---|
| Titanium | Ti(IV)O₂, 99%* | 25 nm | Sigma-Aldrich | 334662 | 13463-67-7 | 79.9 |
| Dioxide | | | | | | |
| Carbon Black | Monarch 880* | 16 nm | Cabot Corp. | | 1333-86-4 | |
| Carbon Black | Printex 90* | 14 nm | Degussa Corp. | | 1333-86-4 | |
| Carbon Black | FW285* | 11 nm | Degussa Corp. | | 1333-86-4 | |
| Carbonyl Iron | Ferronyl Iron⁺⁺ | 5.88 μm | ISP Technol. | 6140150 | 7439-89-6 | 55.9 |
| Quartz | Min-U-Sil ® 5** | 1.6 μm | U.S. Silica | | 14808-60-7 | |

*can be sterilized by filtering
**can be sterilized by heating
⁺⁺cannot be sterilized by filtering or heating

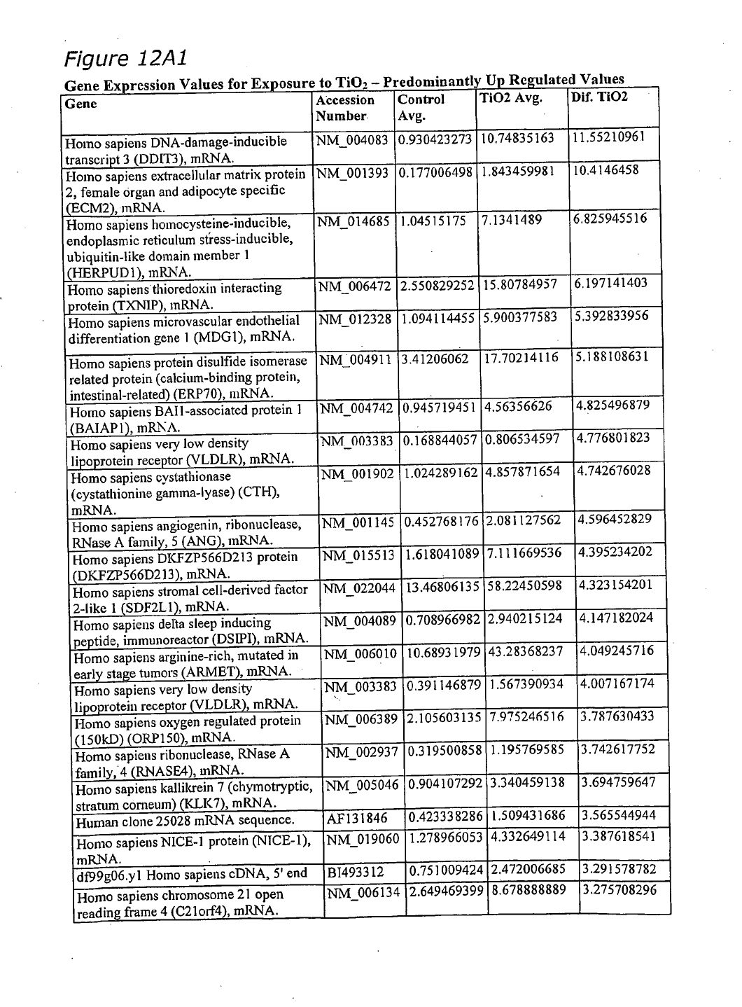

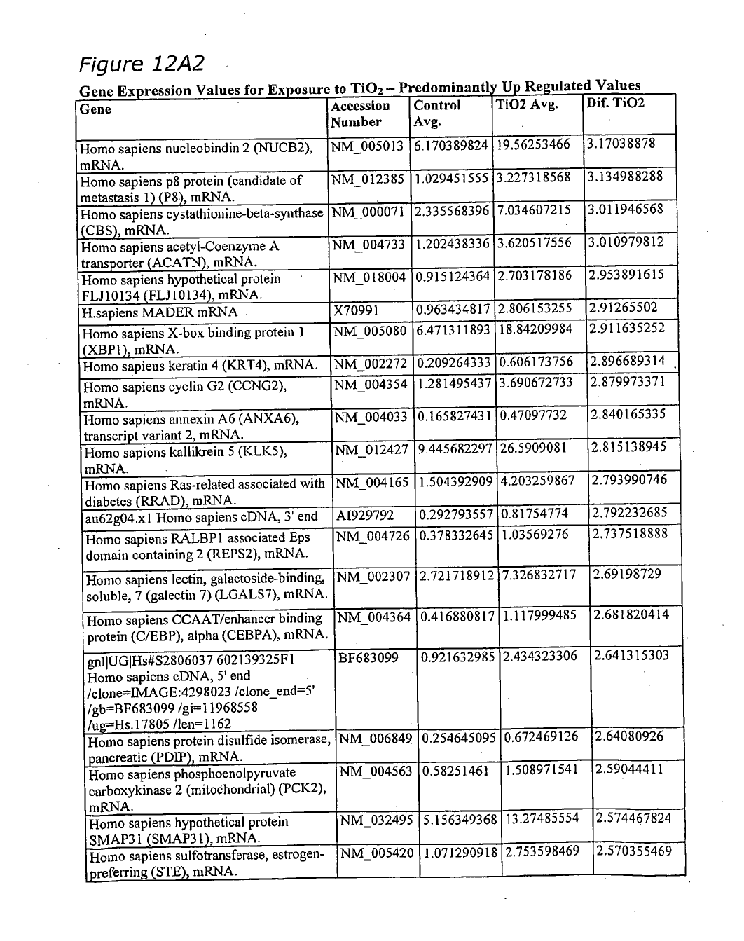

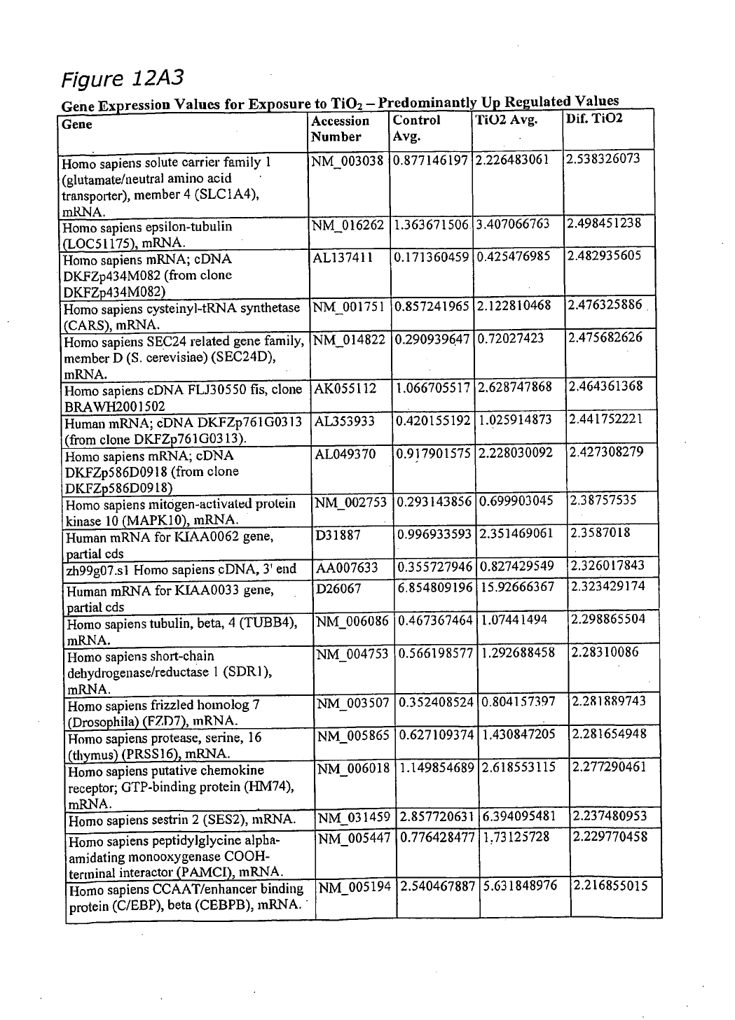

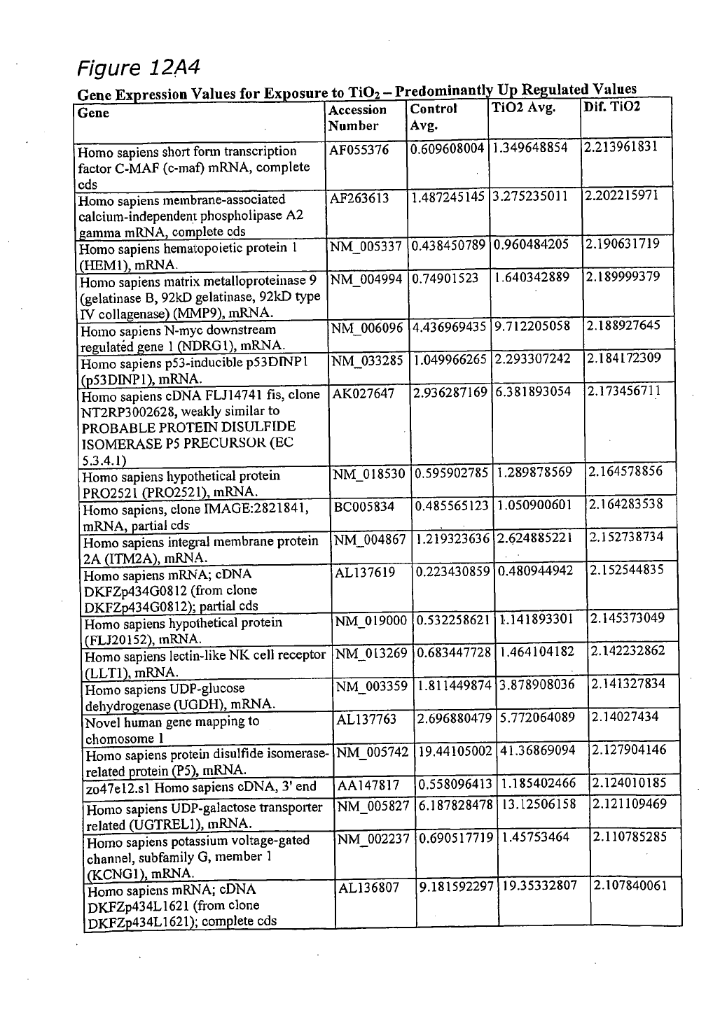

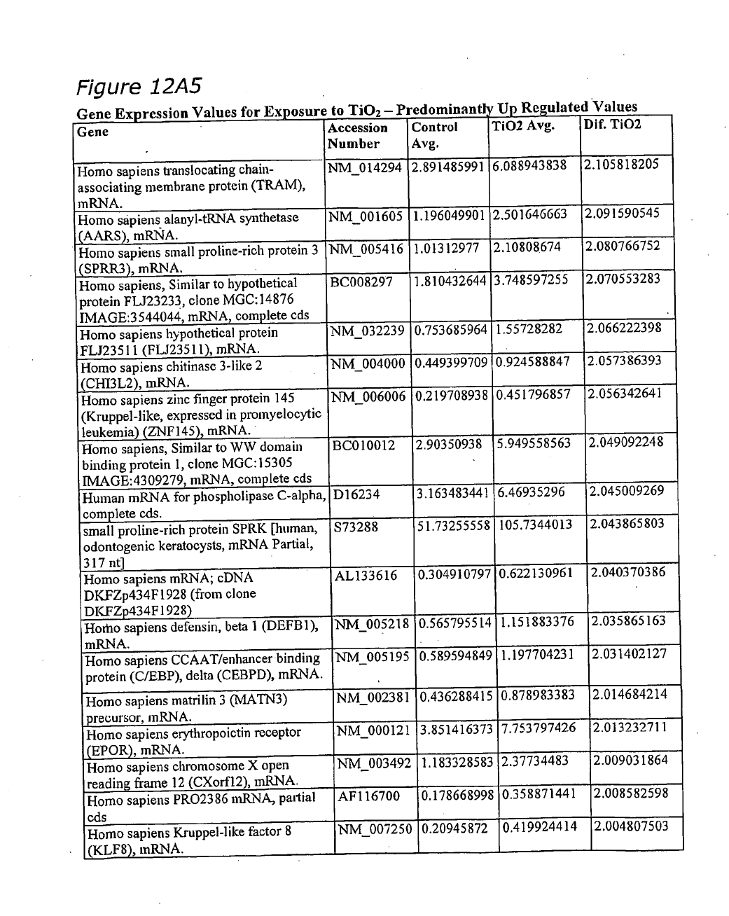

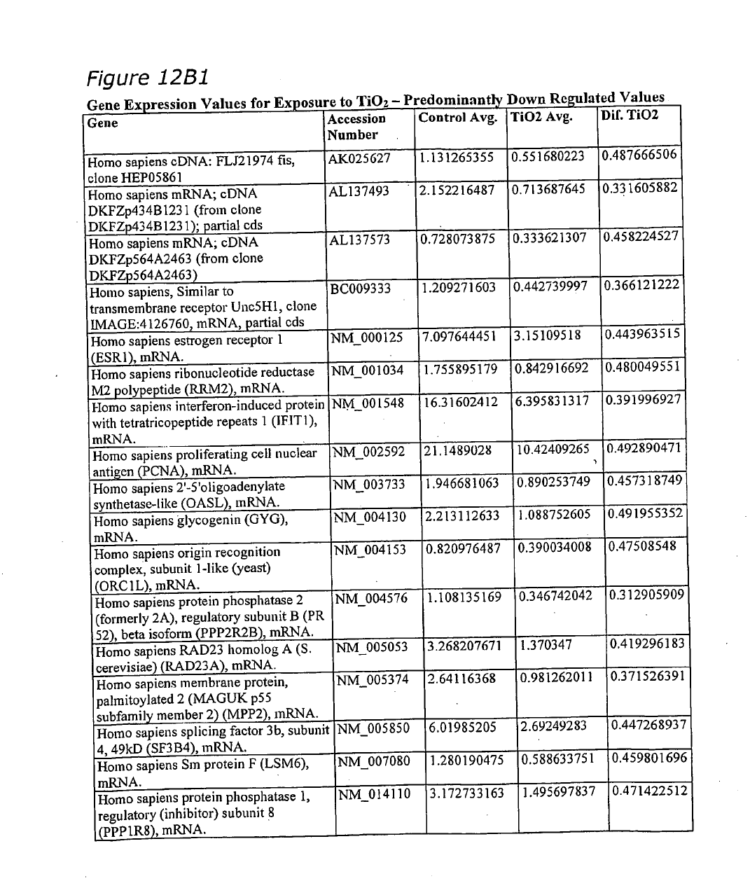

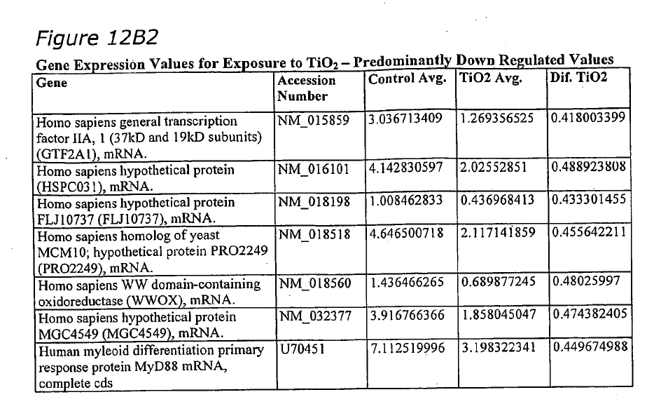

FIG. 12A1-5 presents GEM results for genes predominantly up-regulated in response to TiO₂ nanoparticle exposure in HEK cells.

FIG. 12B1-2 presents GEM results for genes predominantly down-regulated in response to TiO₂ nanoparticle exposure in HEK cells.

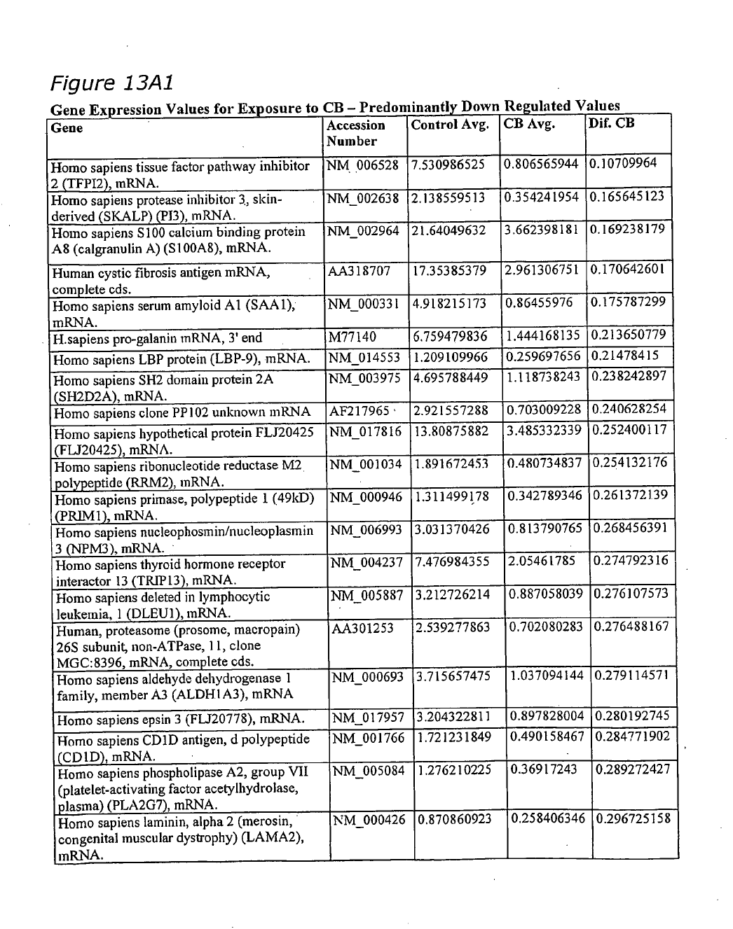

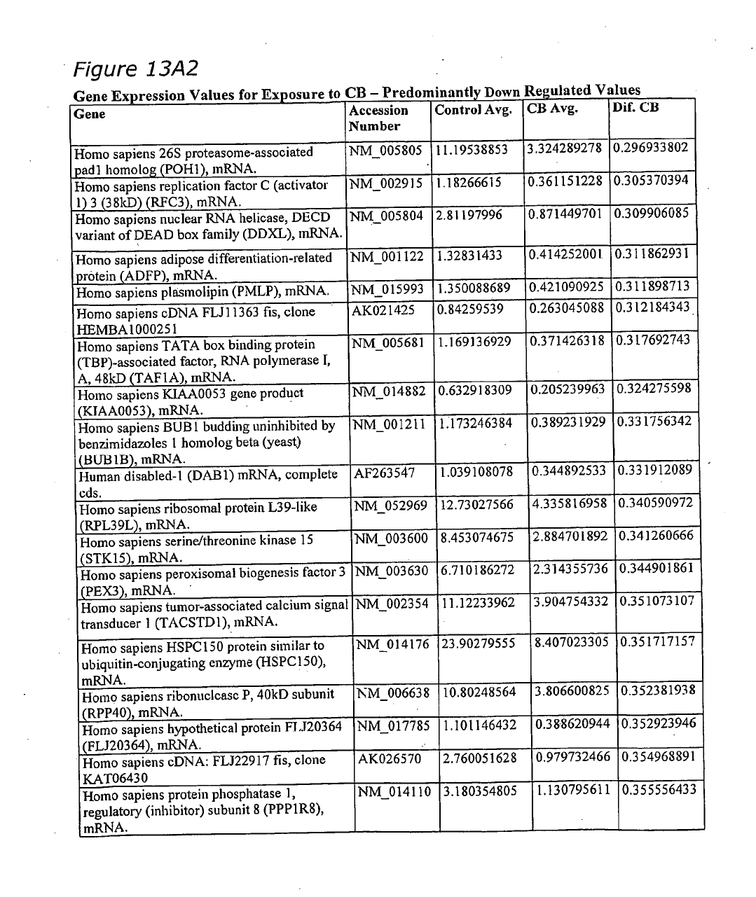

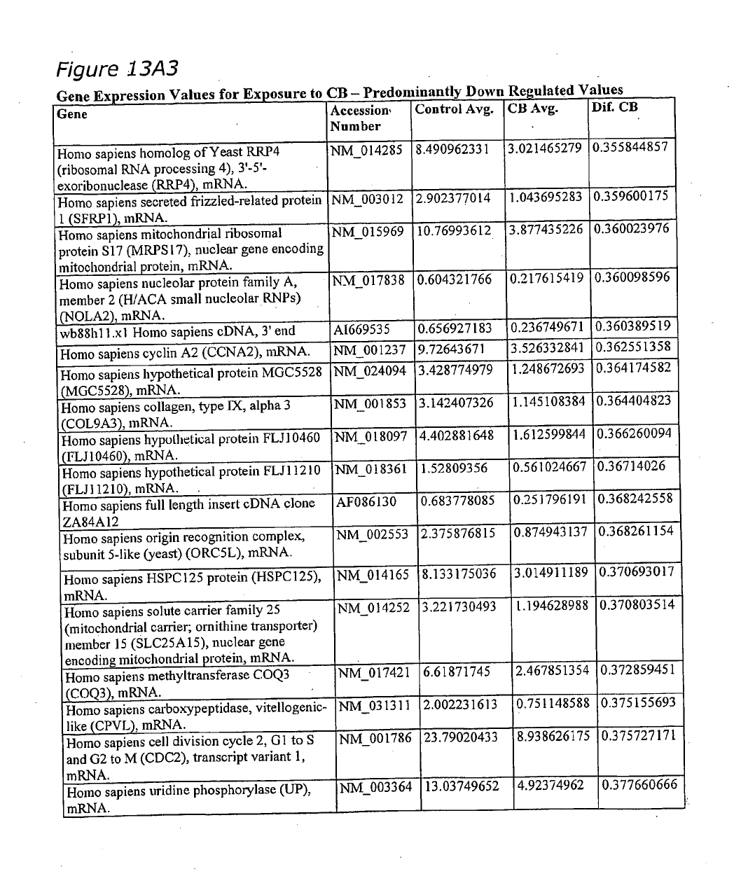

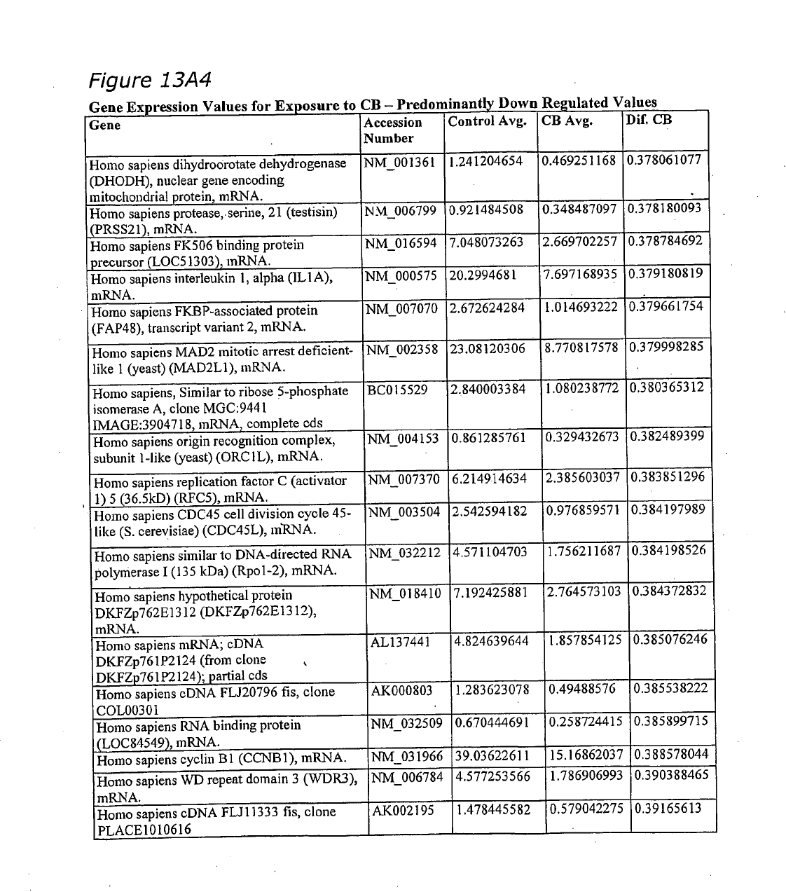

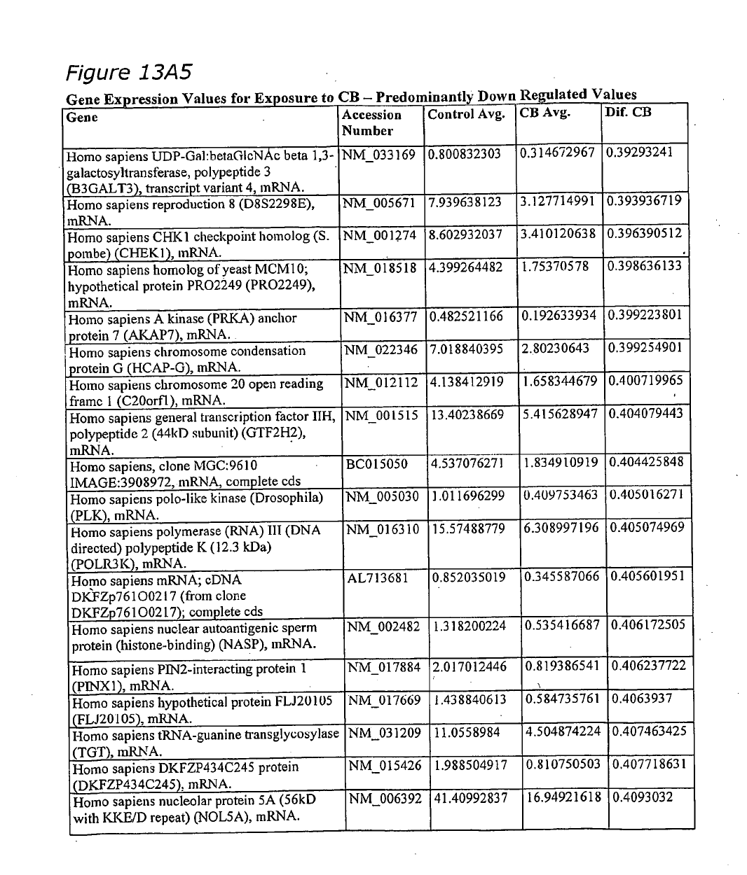

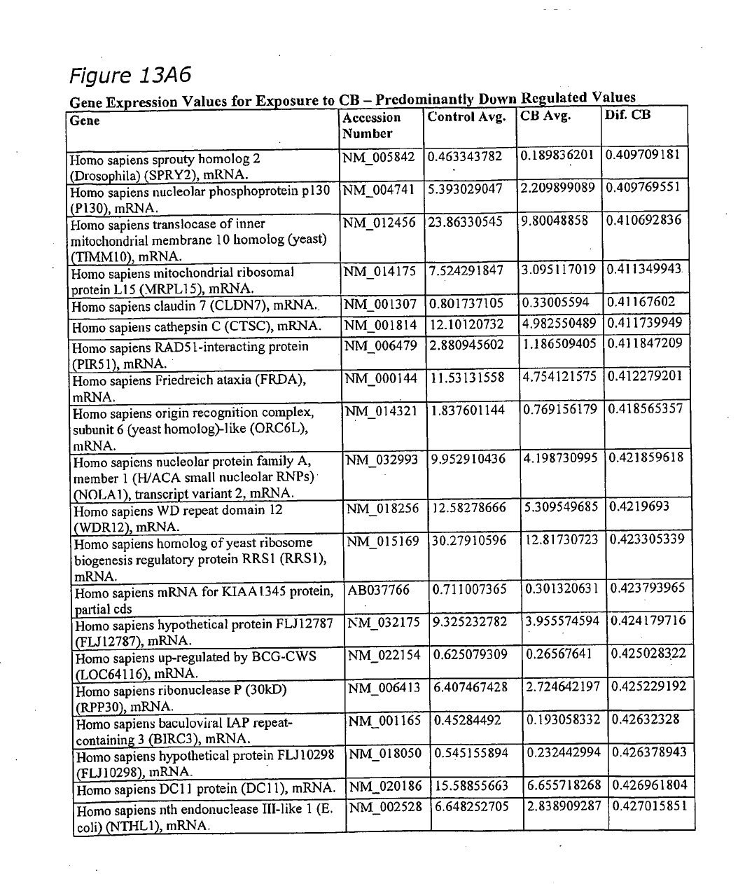

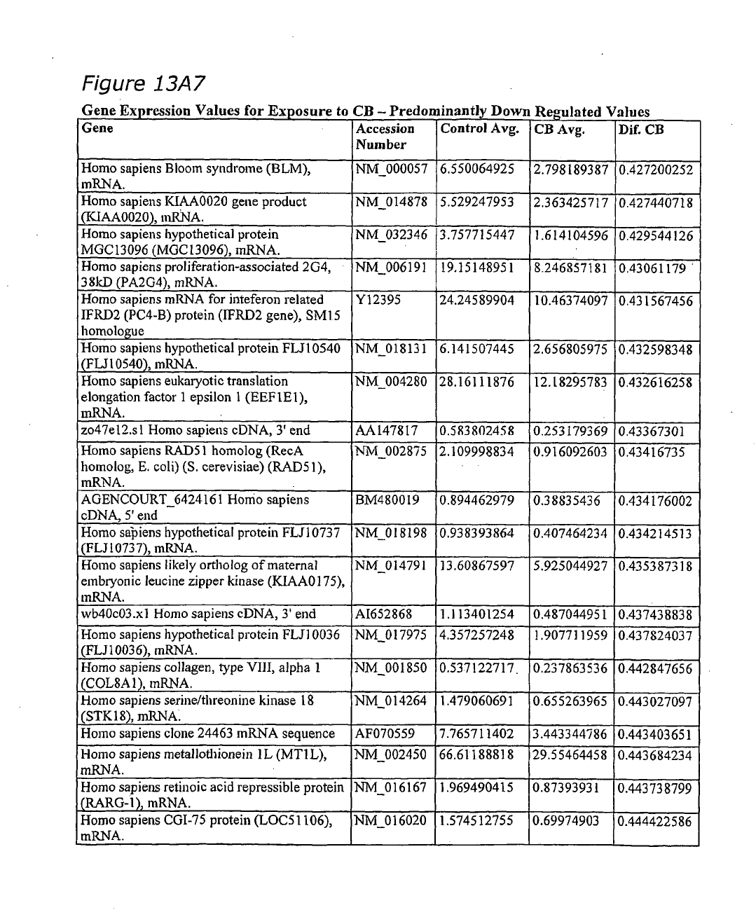

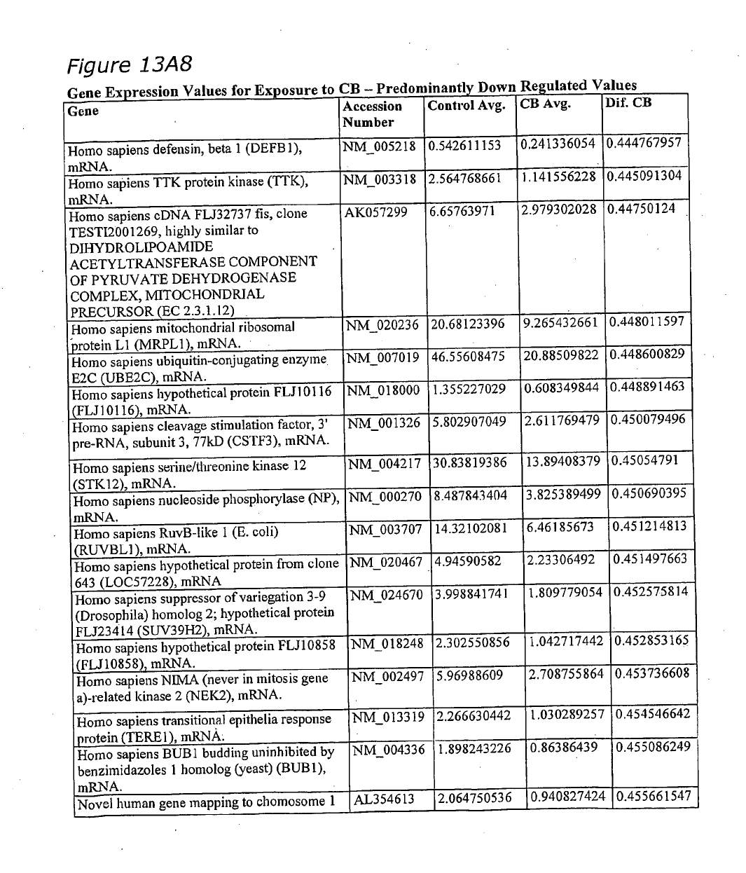

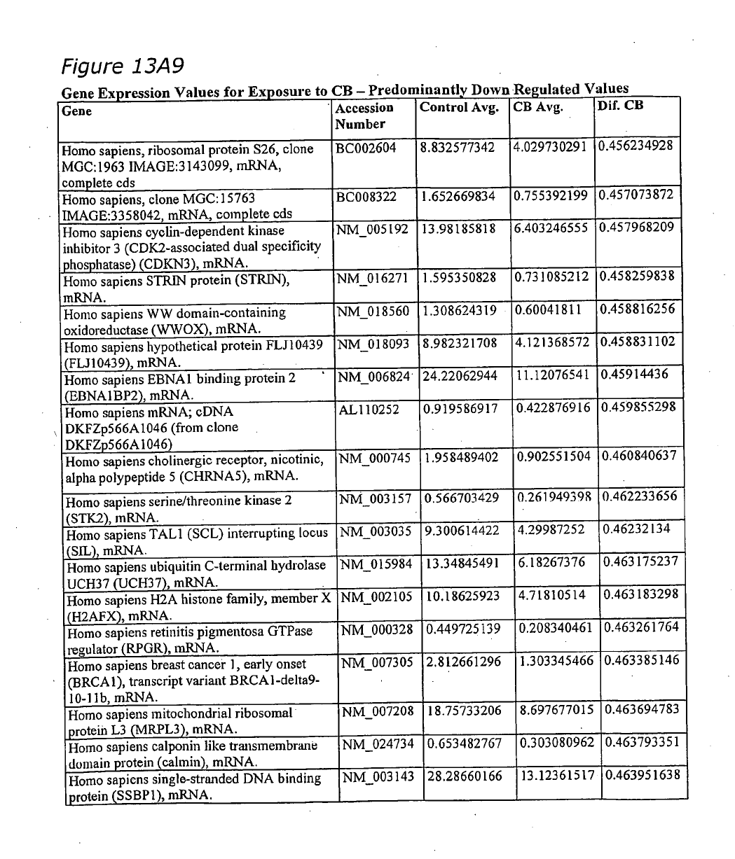

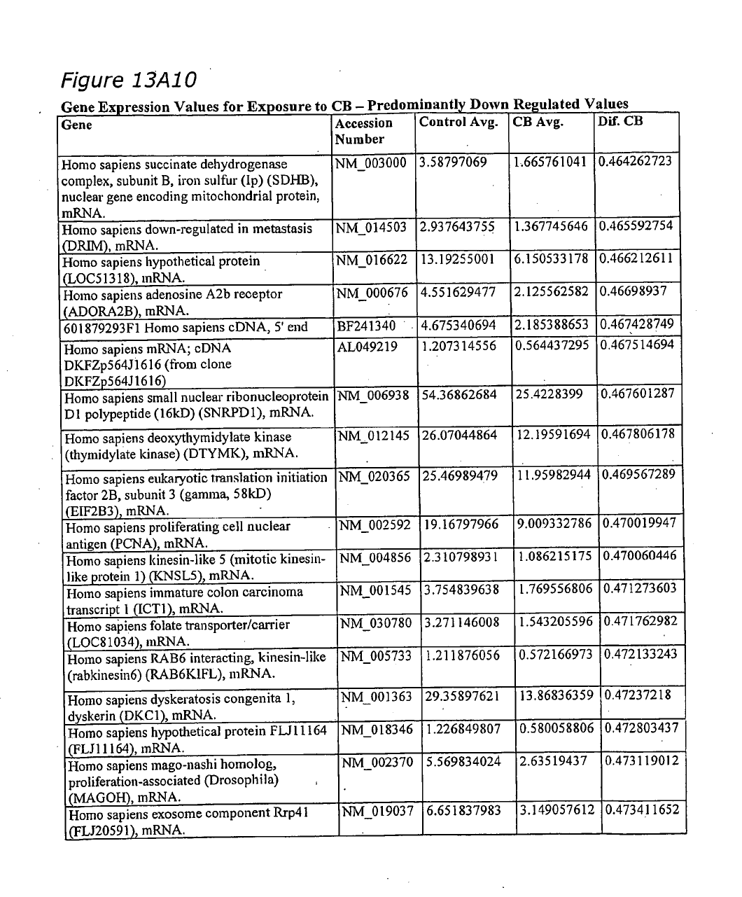

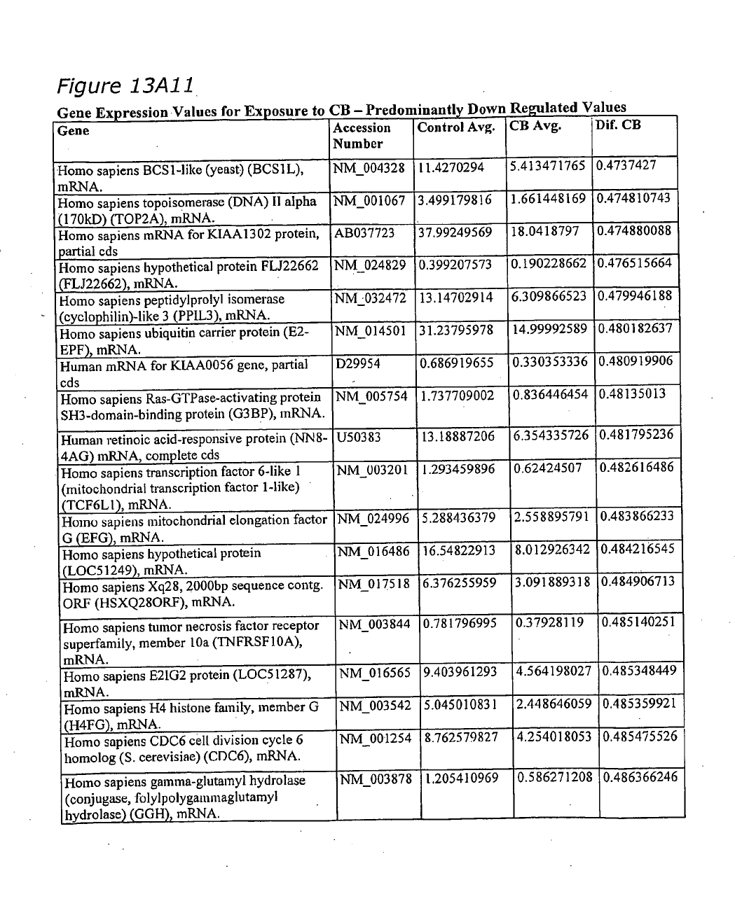

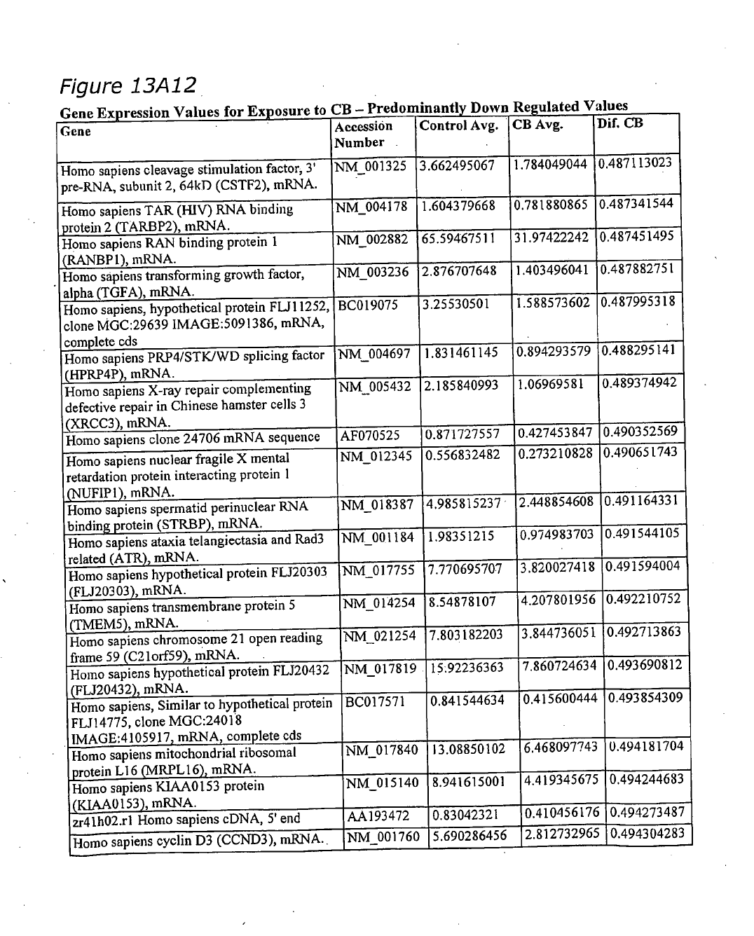

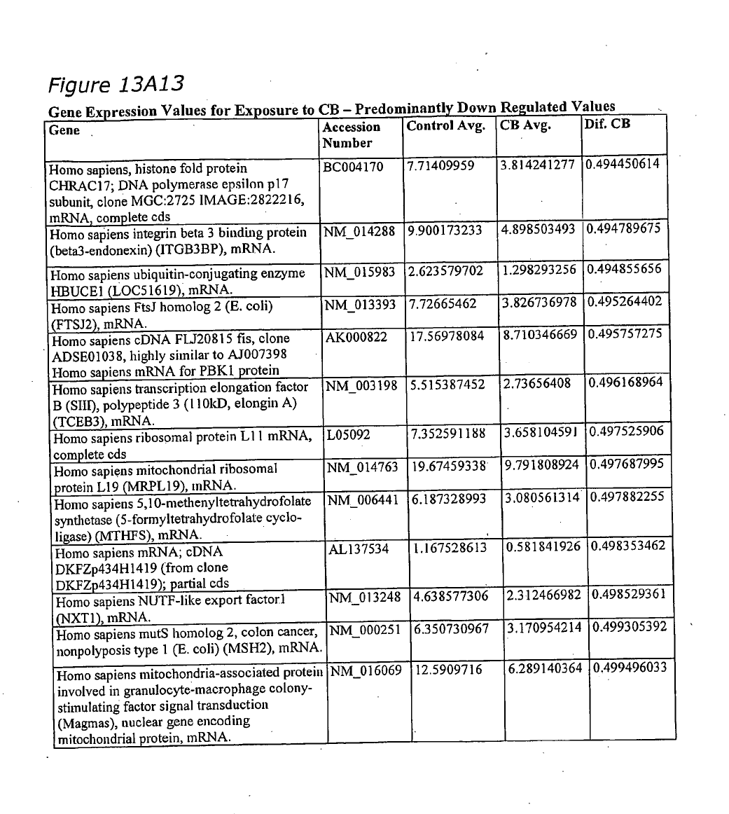

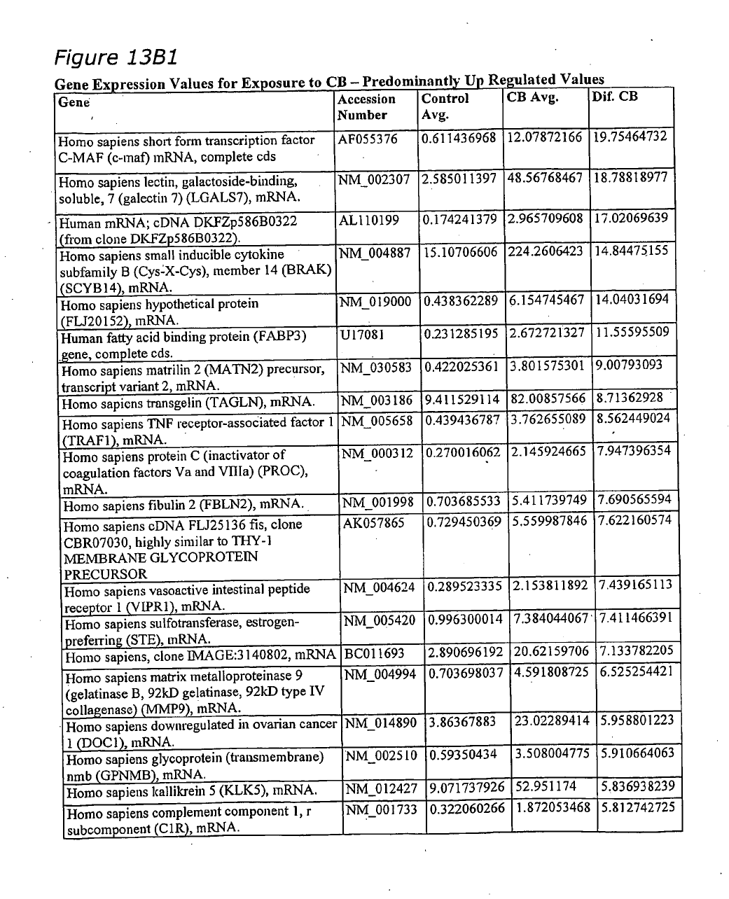

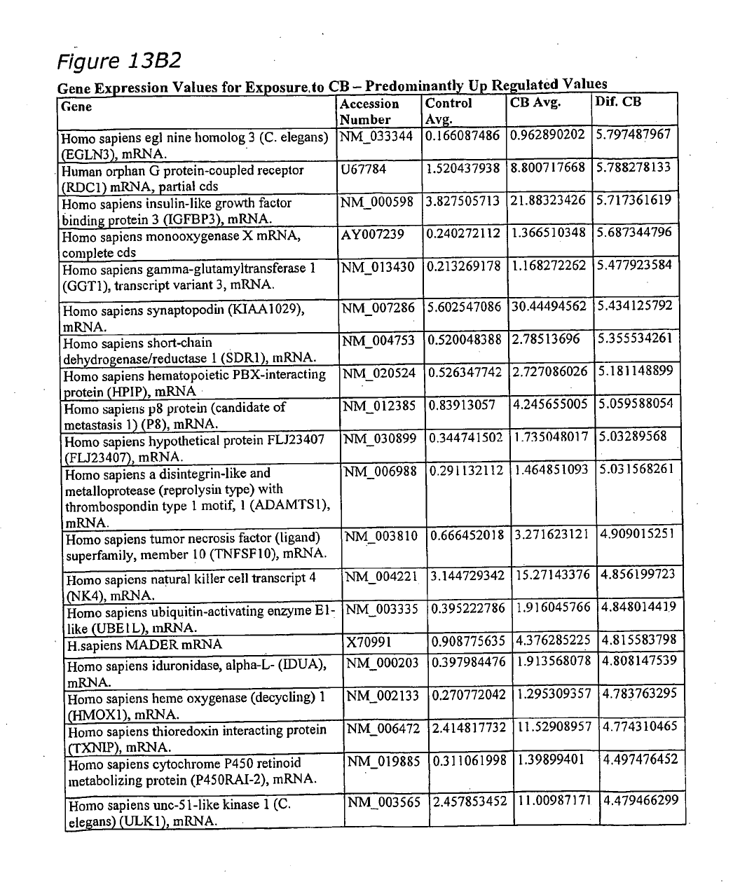

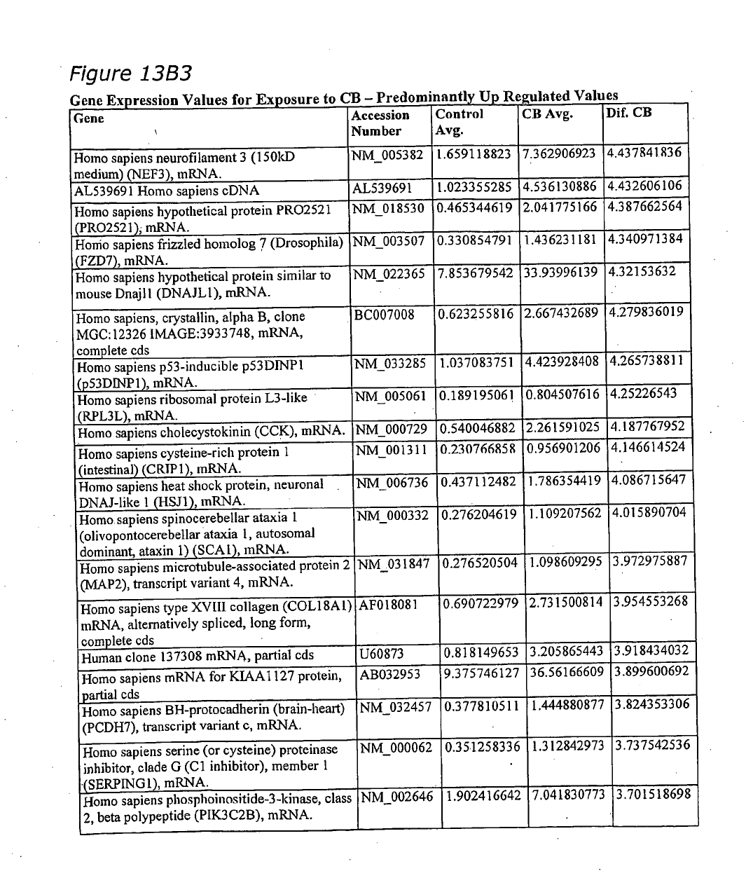

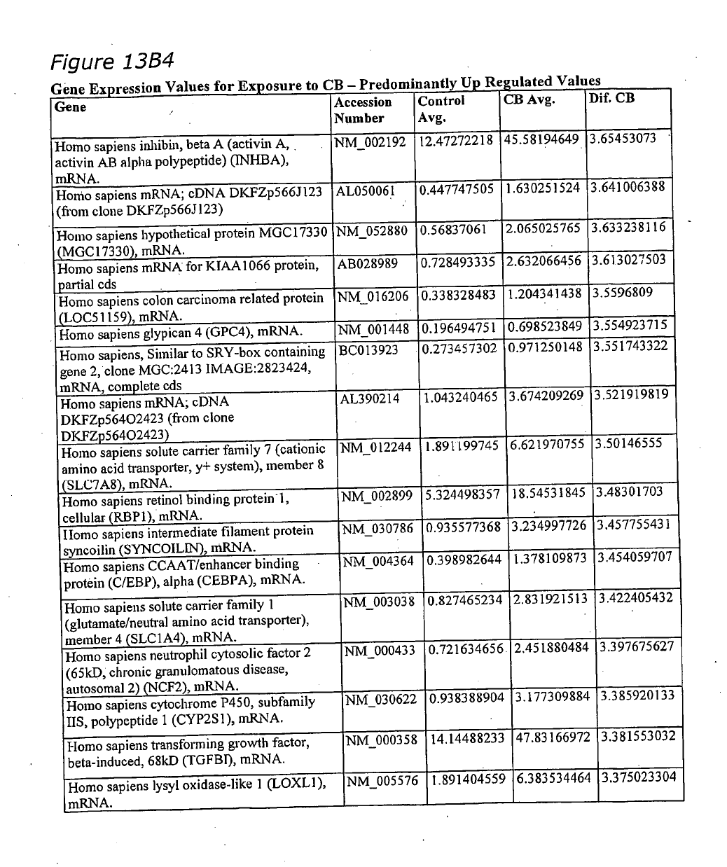

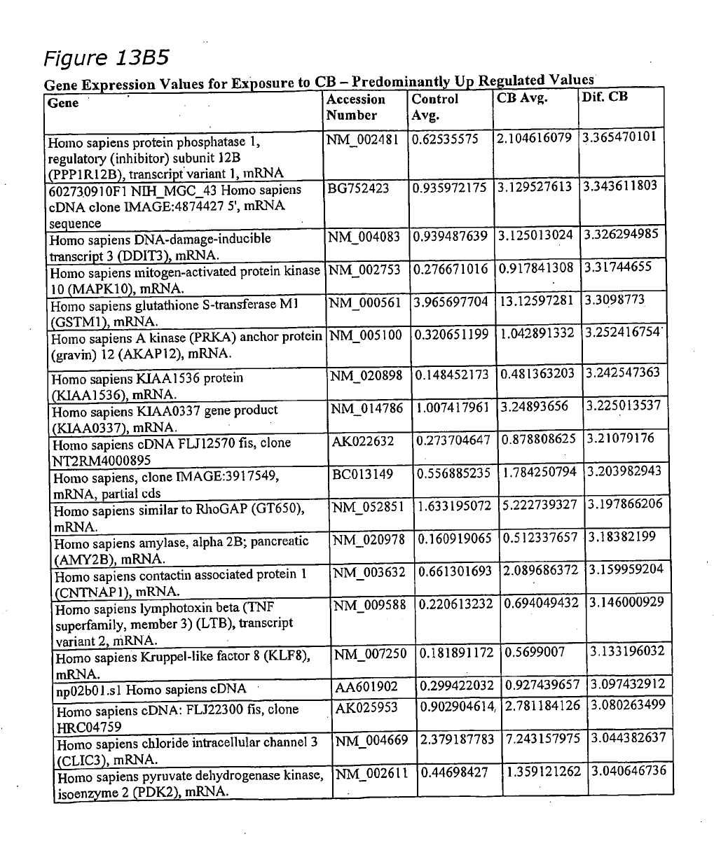

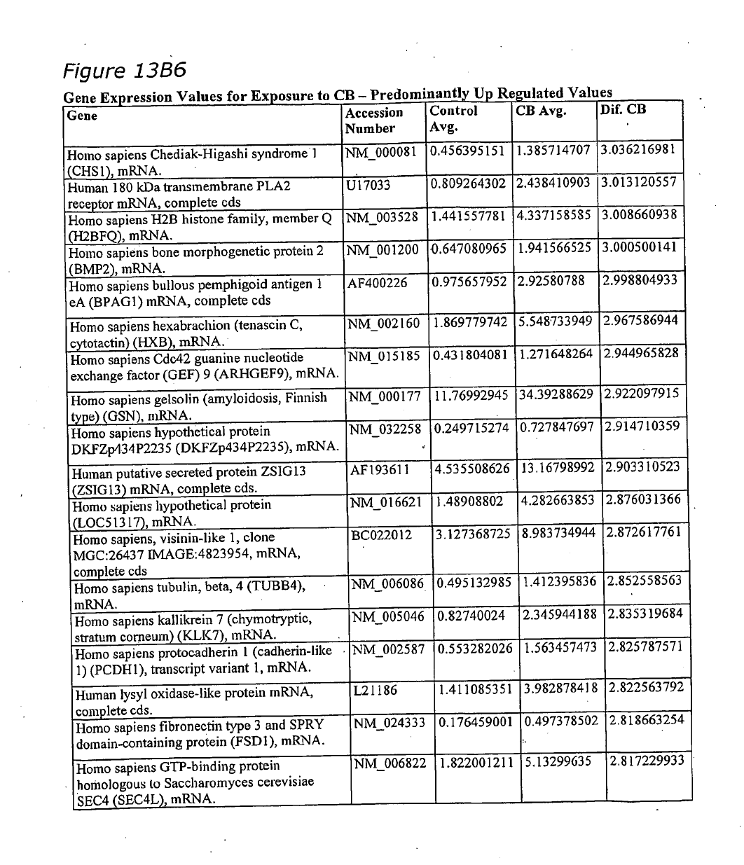

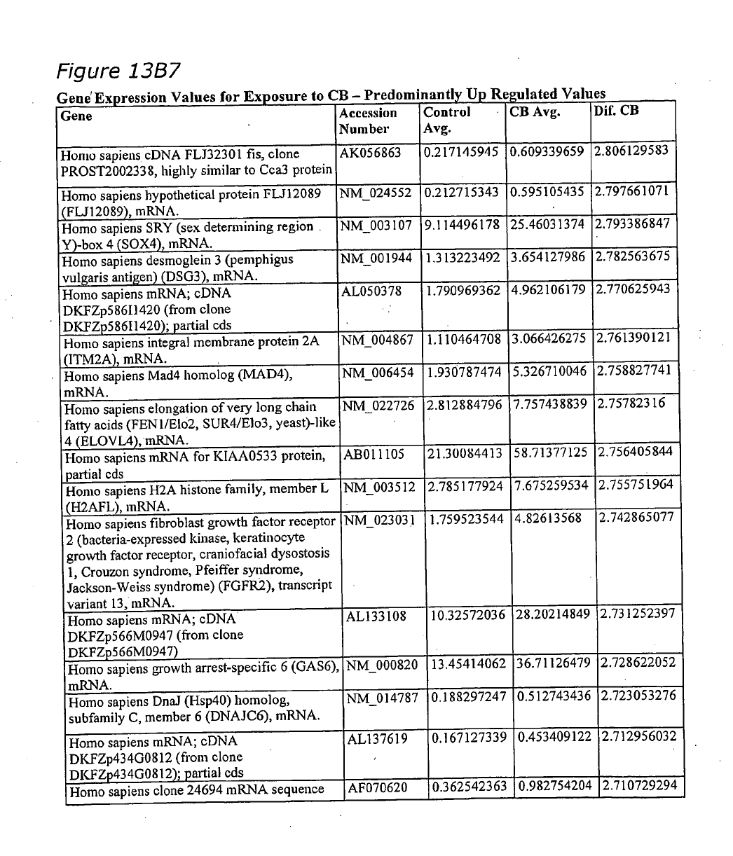

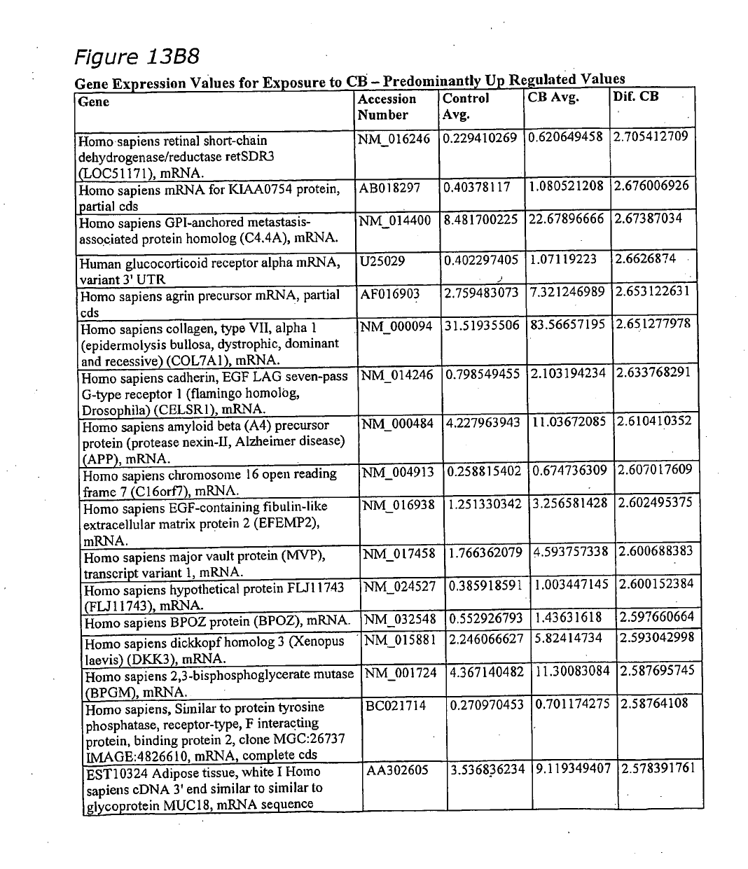

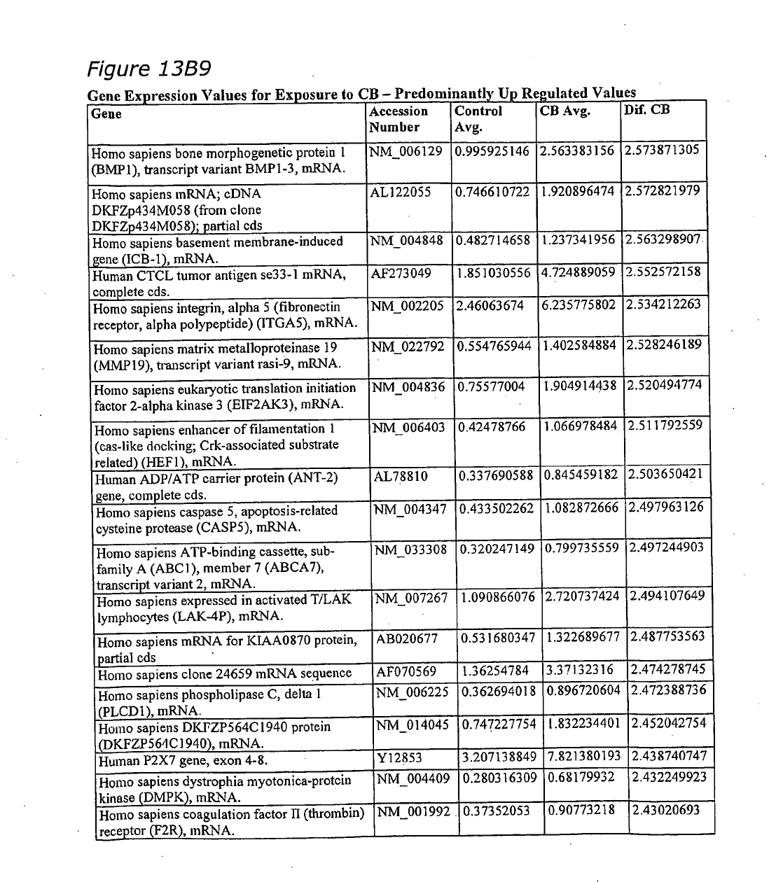

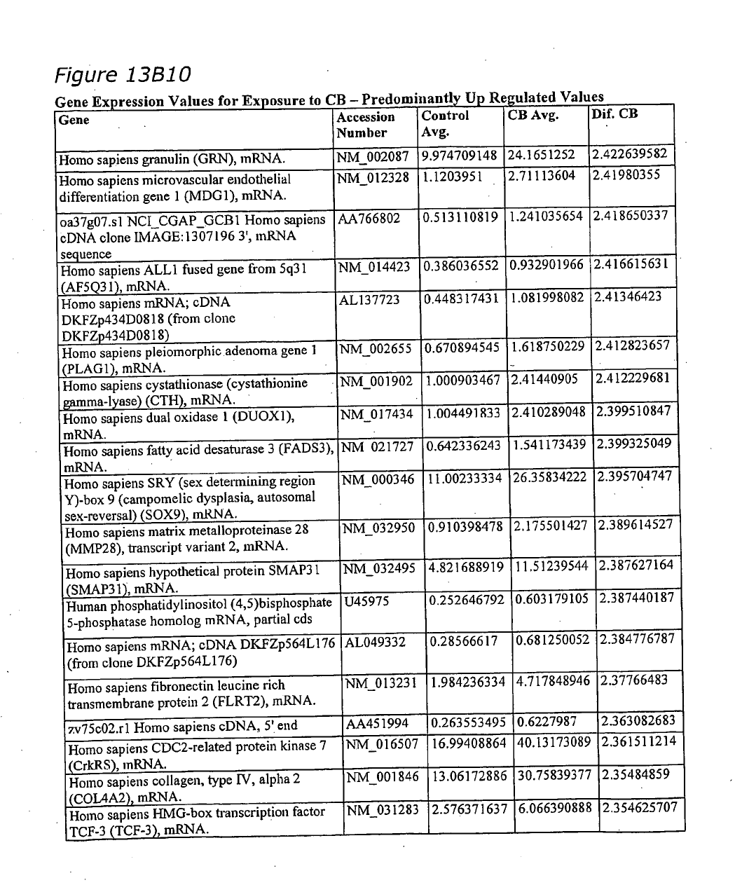

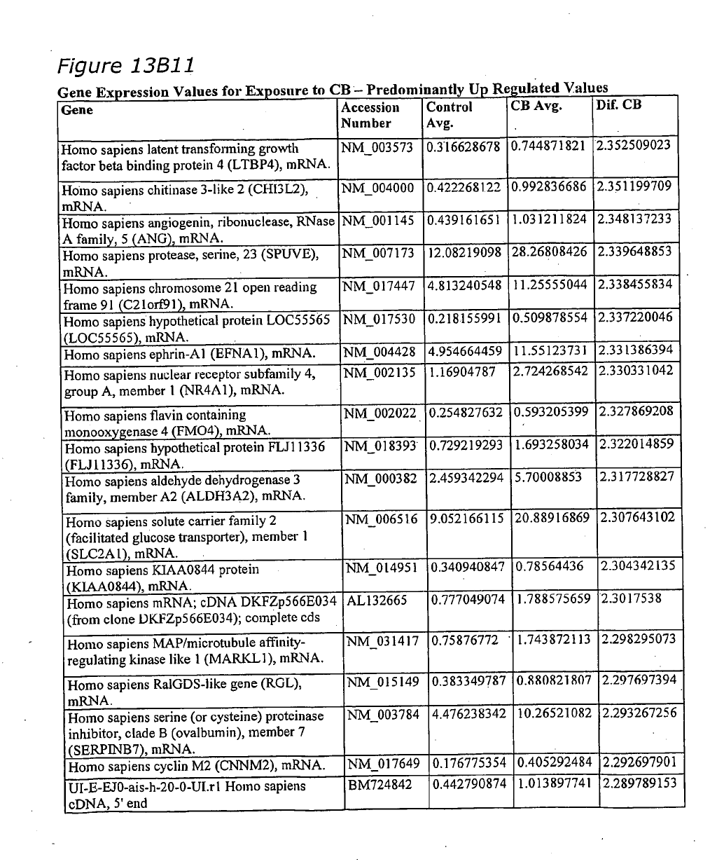

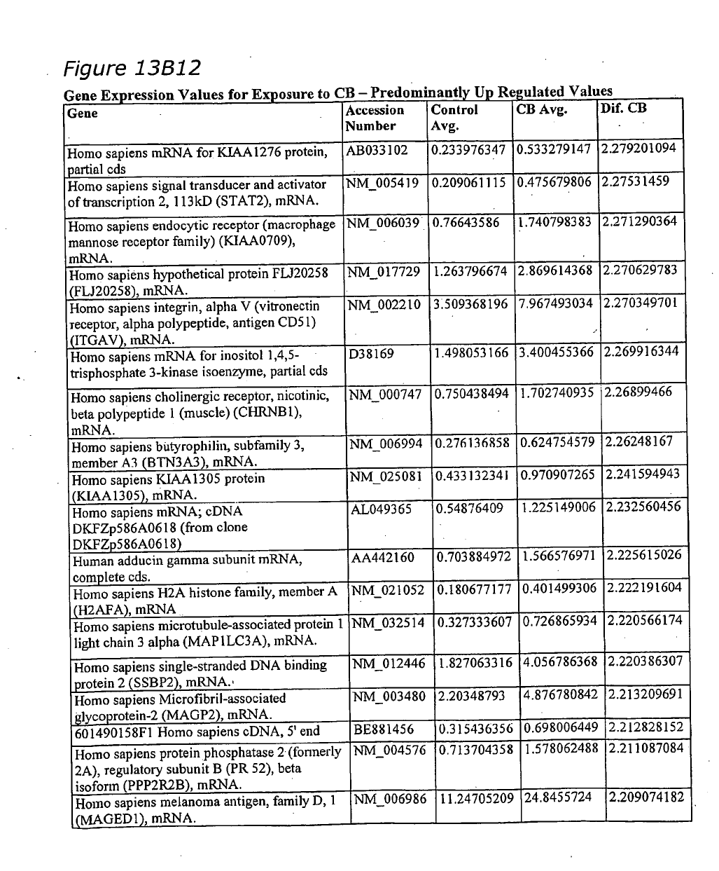

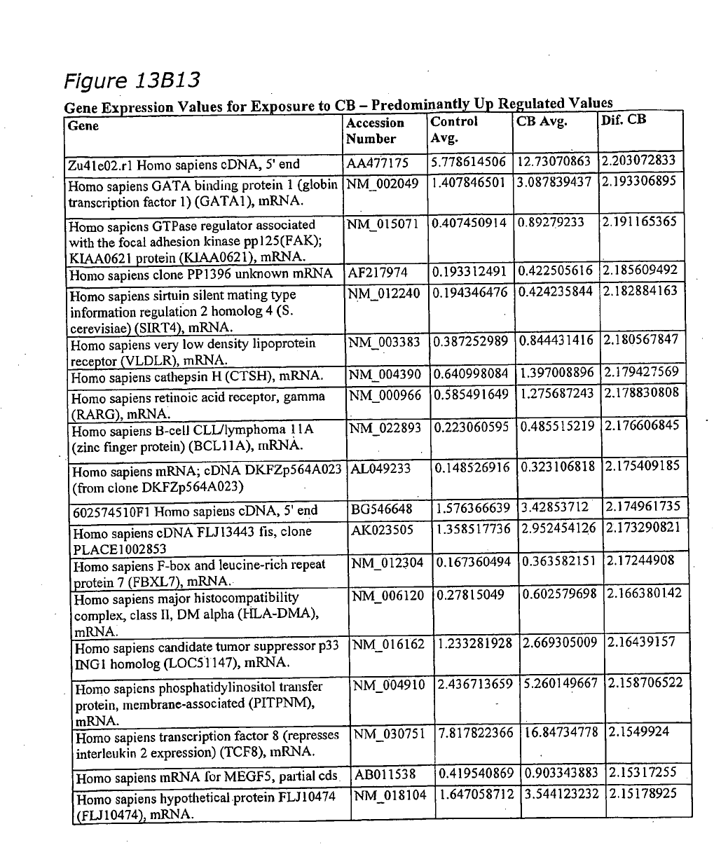

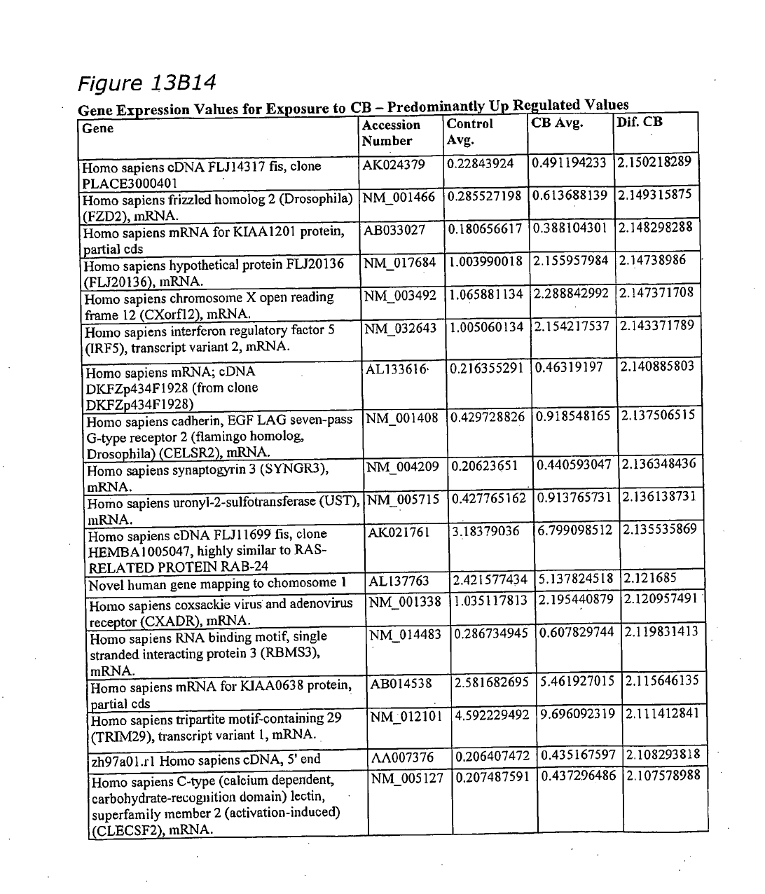

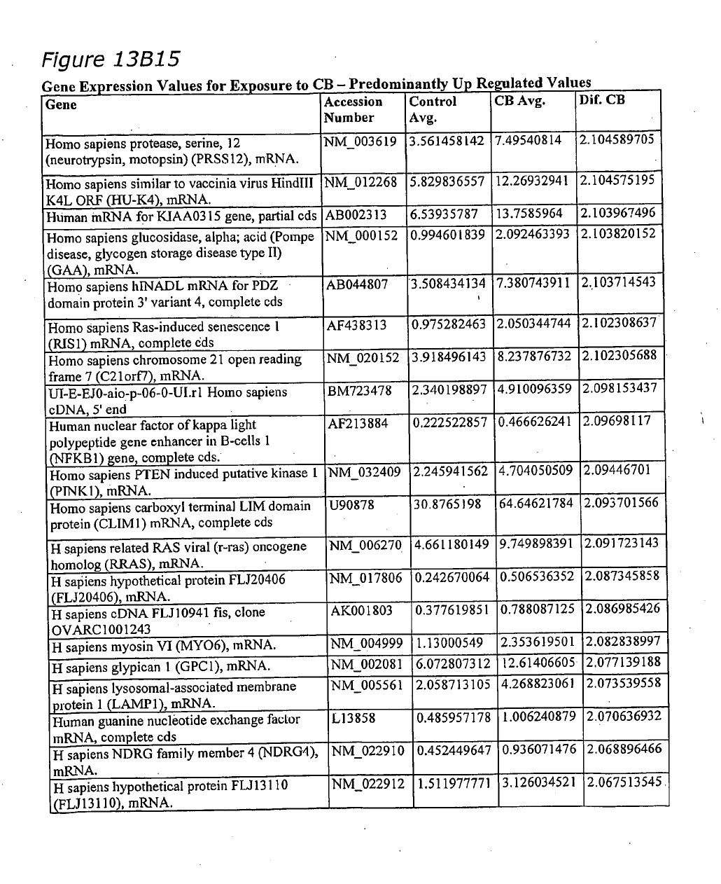

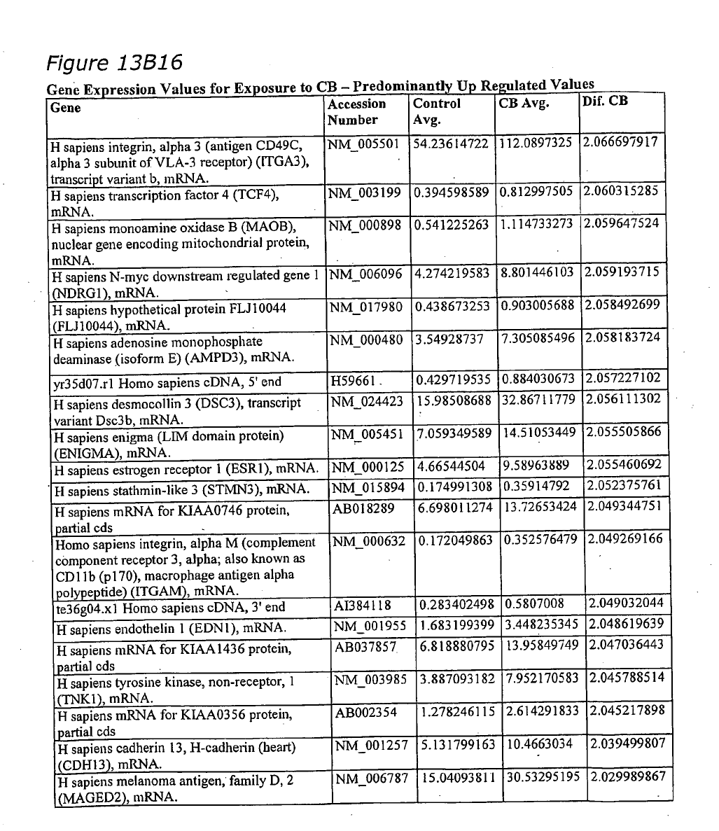

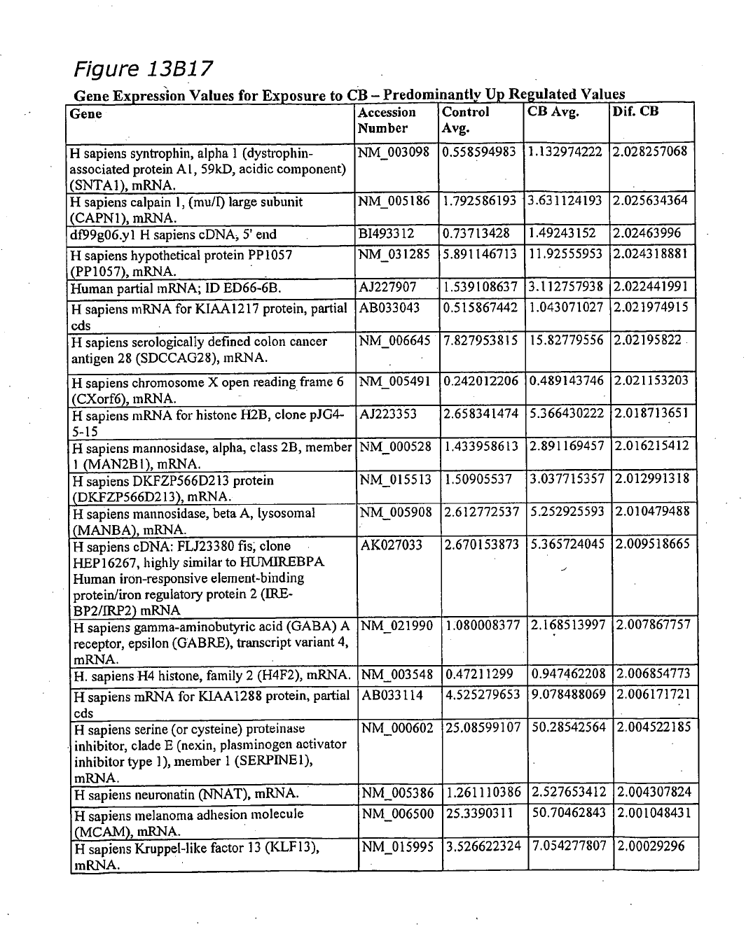

FIG. 13A1-12 presents GEM results for genes predominantly down-regulated in response to CB nanoparticle exposure in HEK cells.

FIG. 13B1-17 presents GEM results for genes predominantly up-regulated in response to CB nanoparticle exposure in HEK cells.

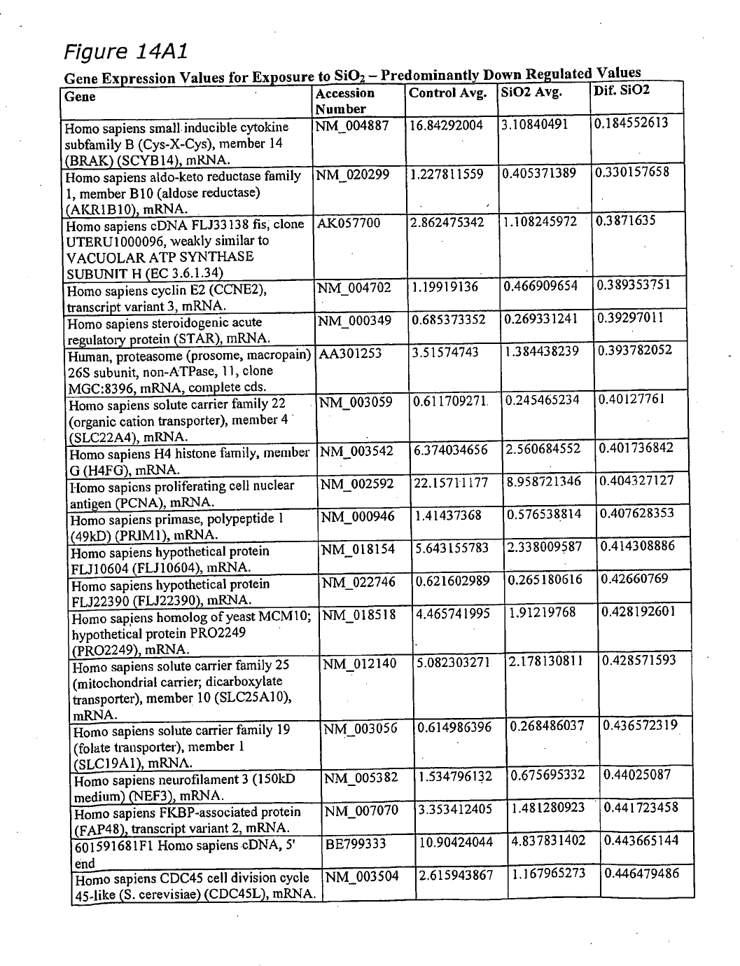

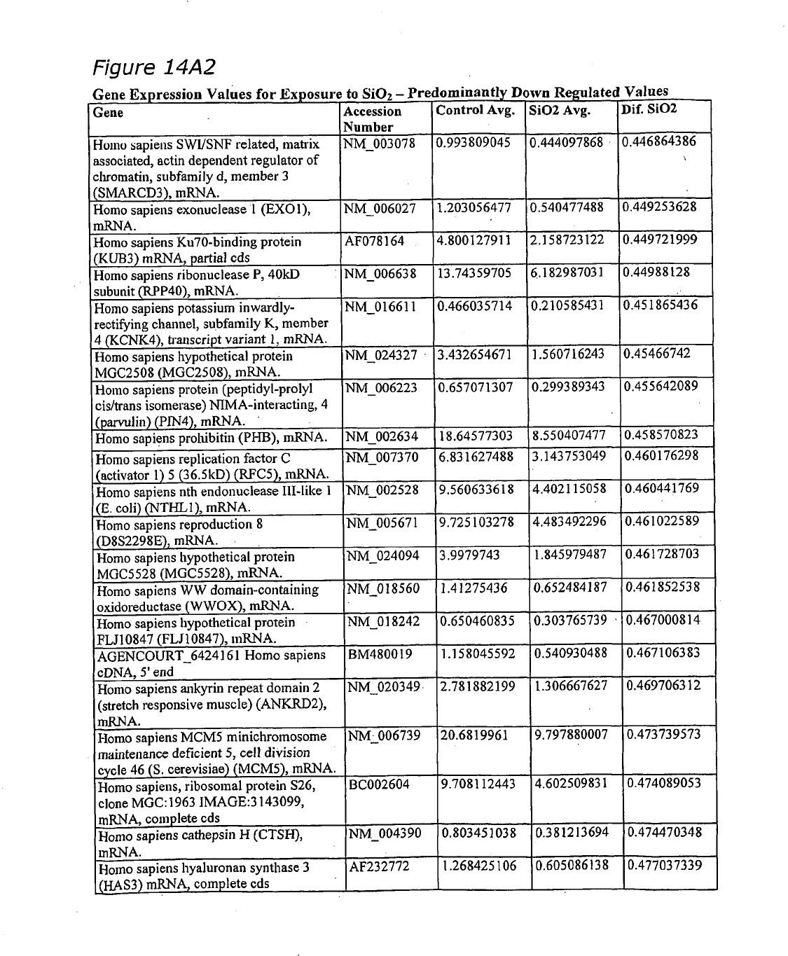

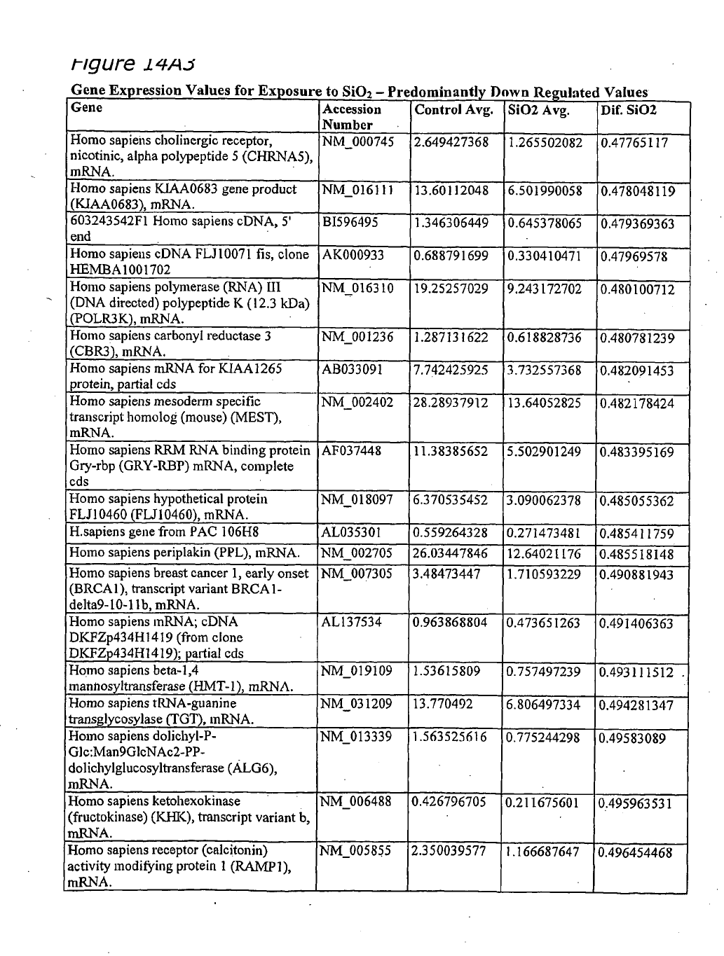

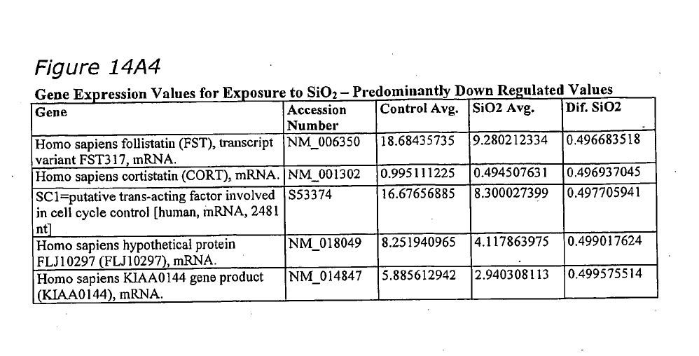

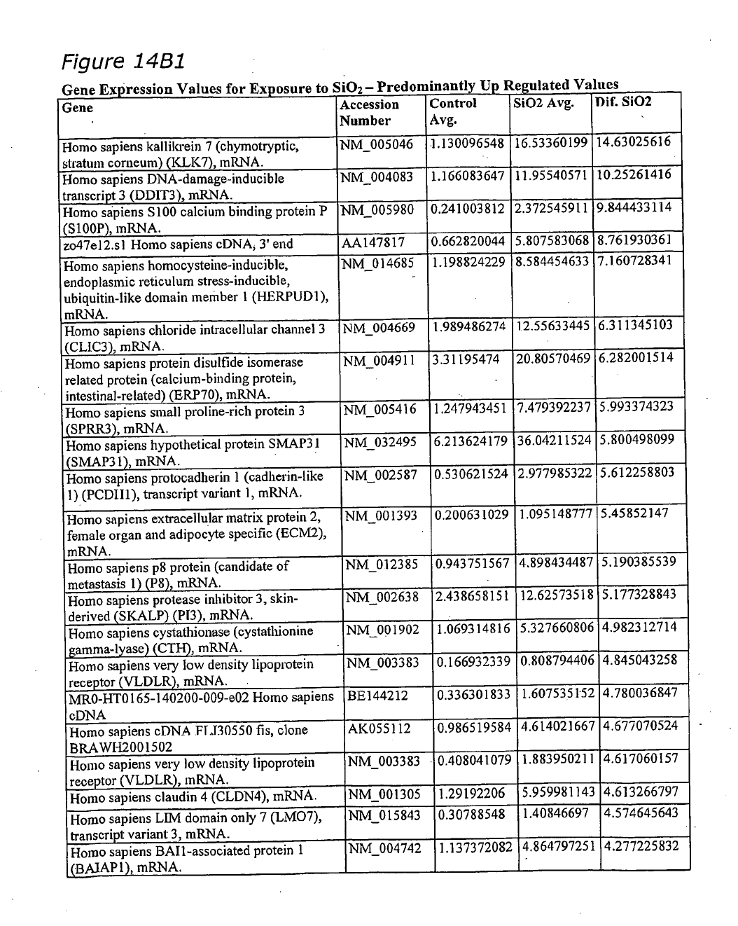

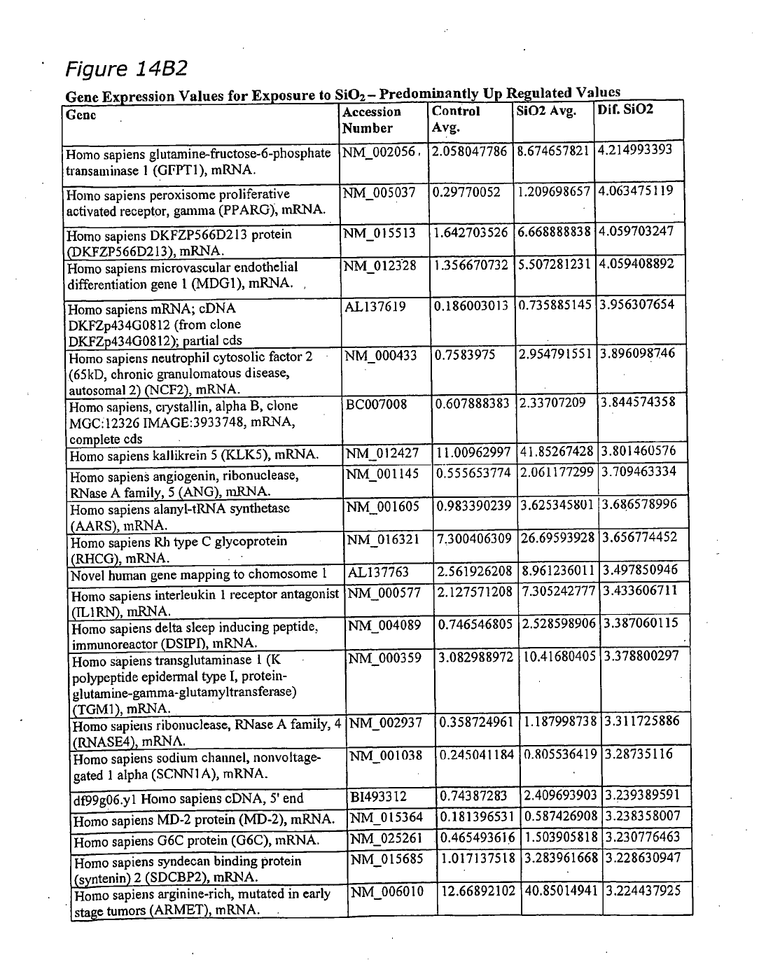

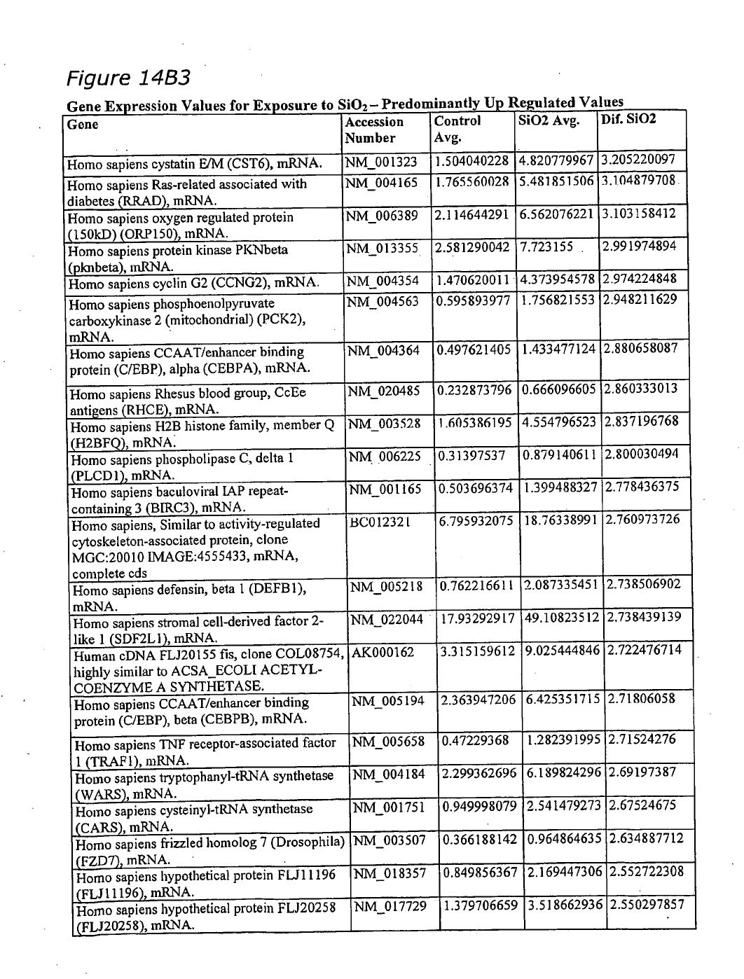

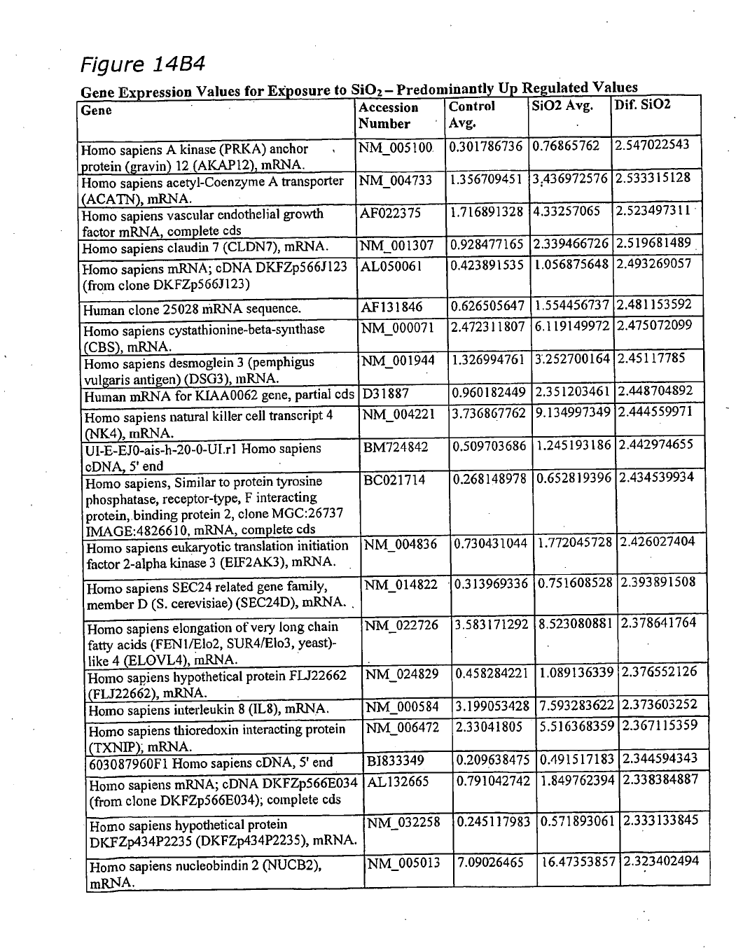

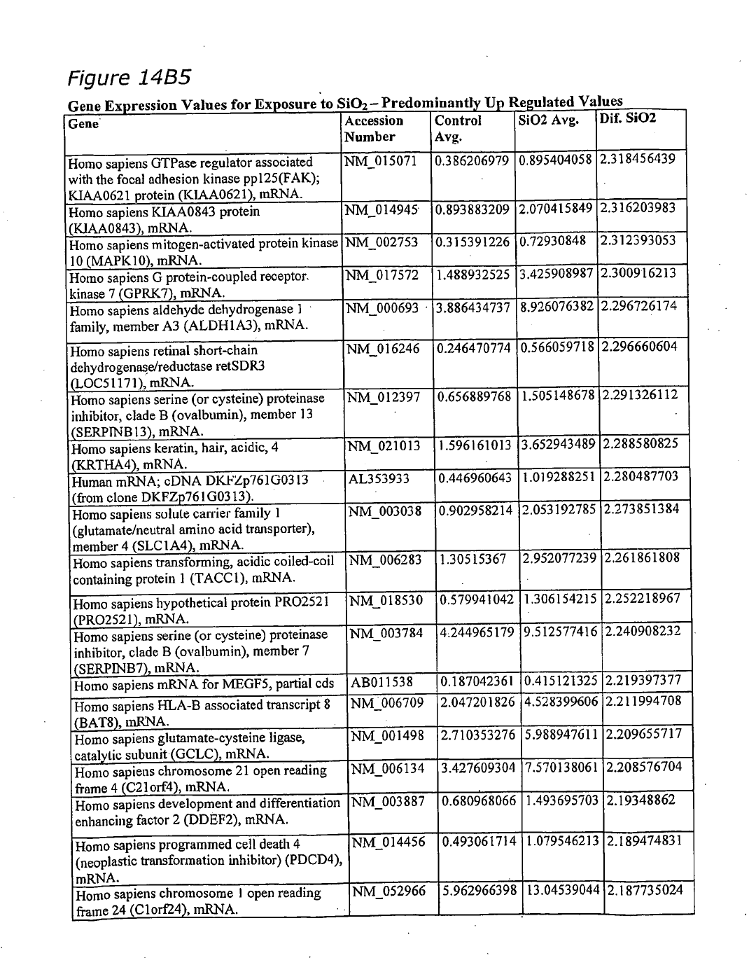

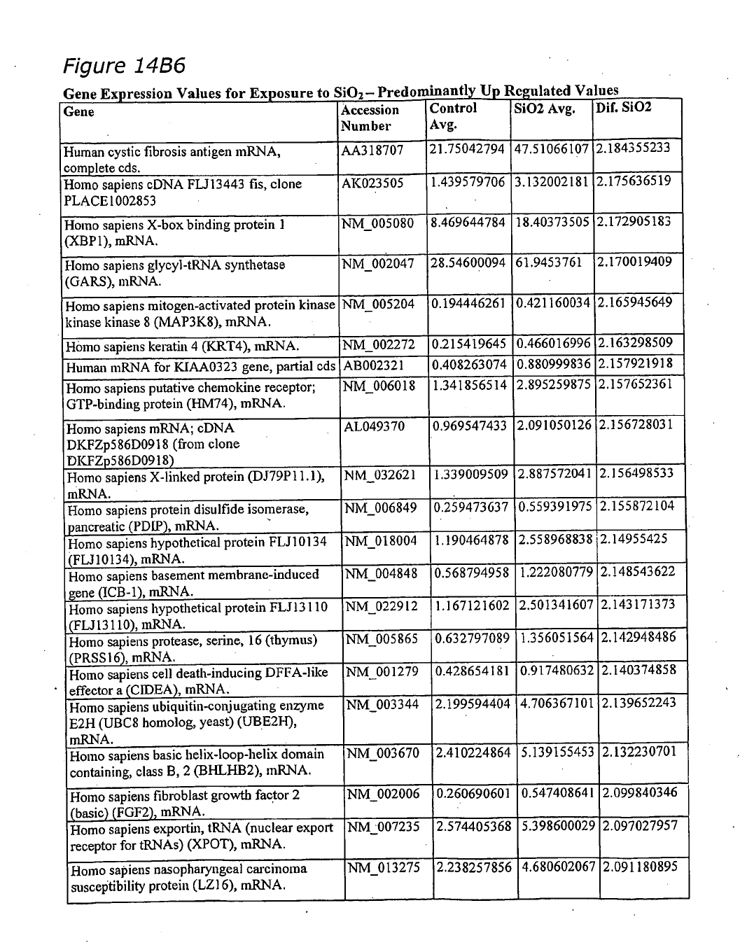

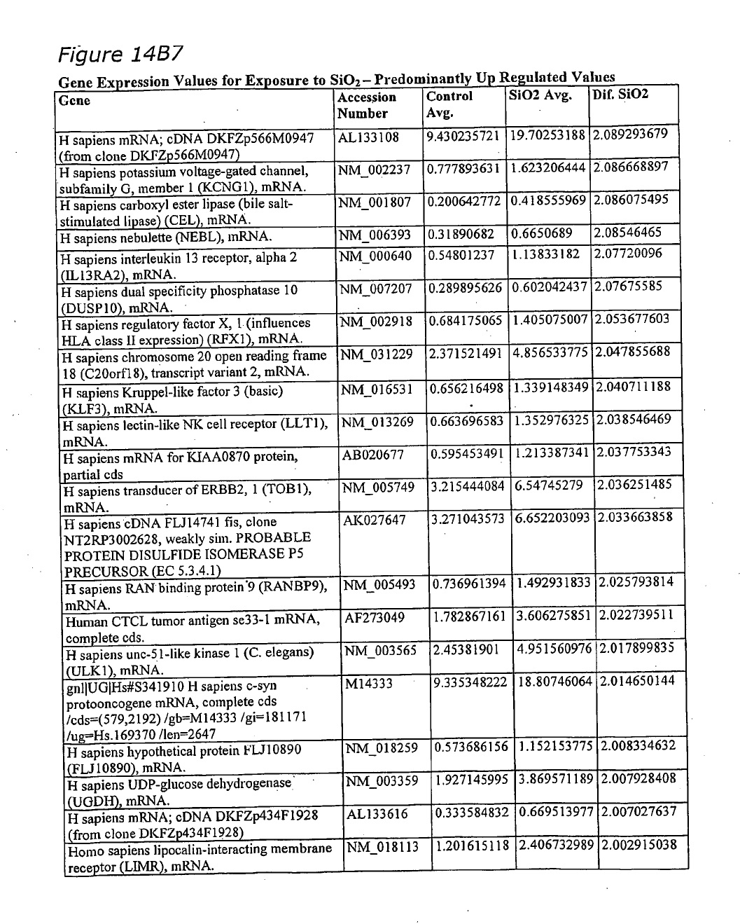

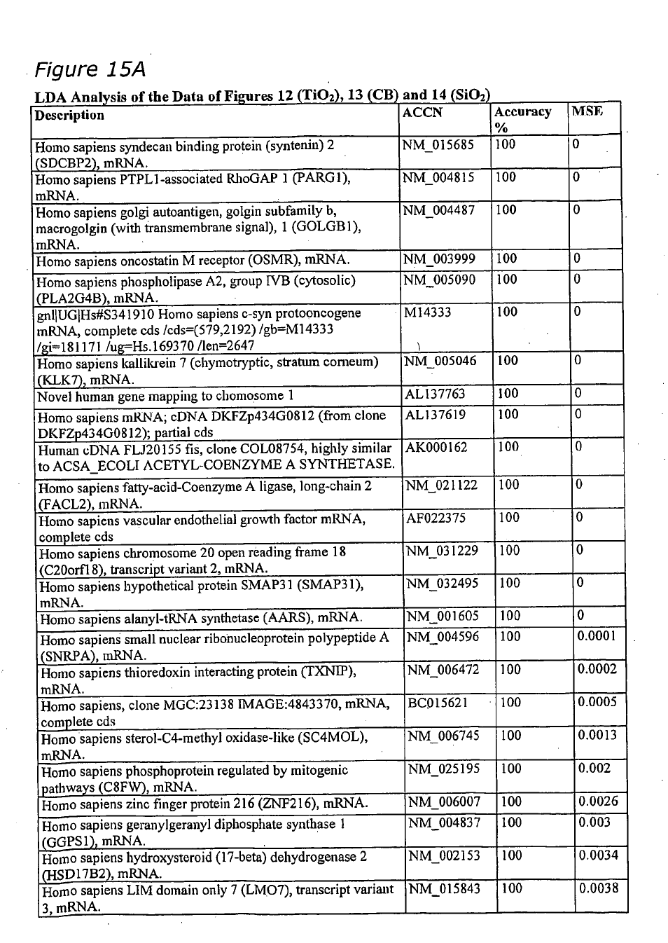

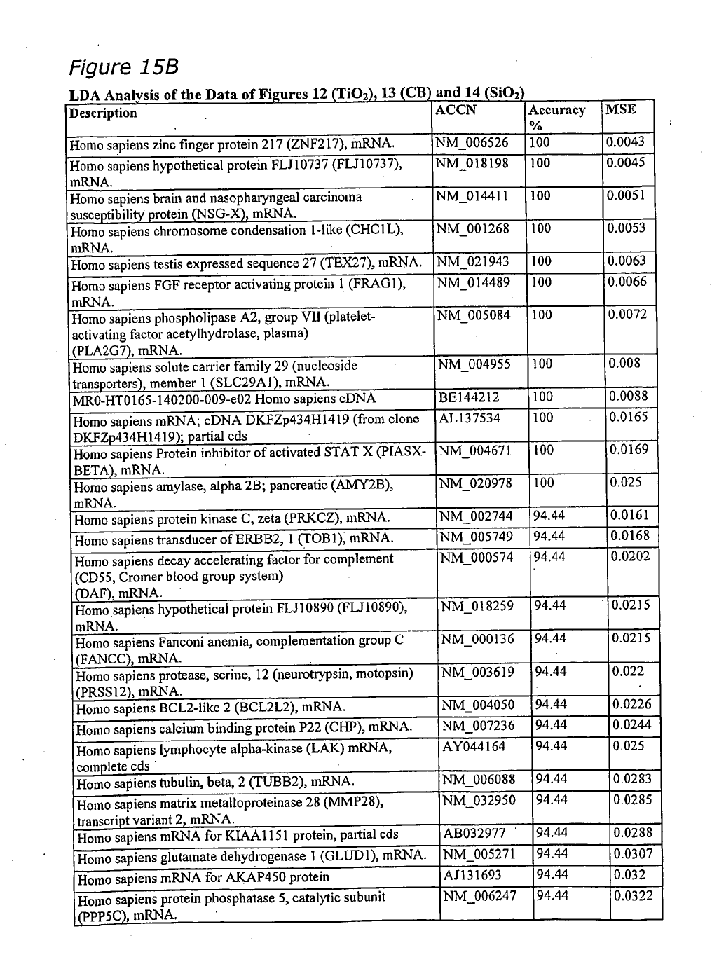

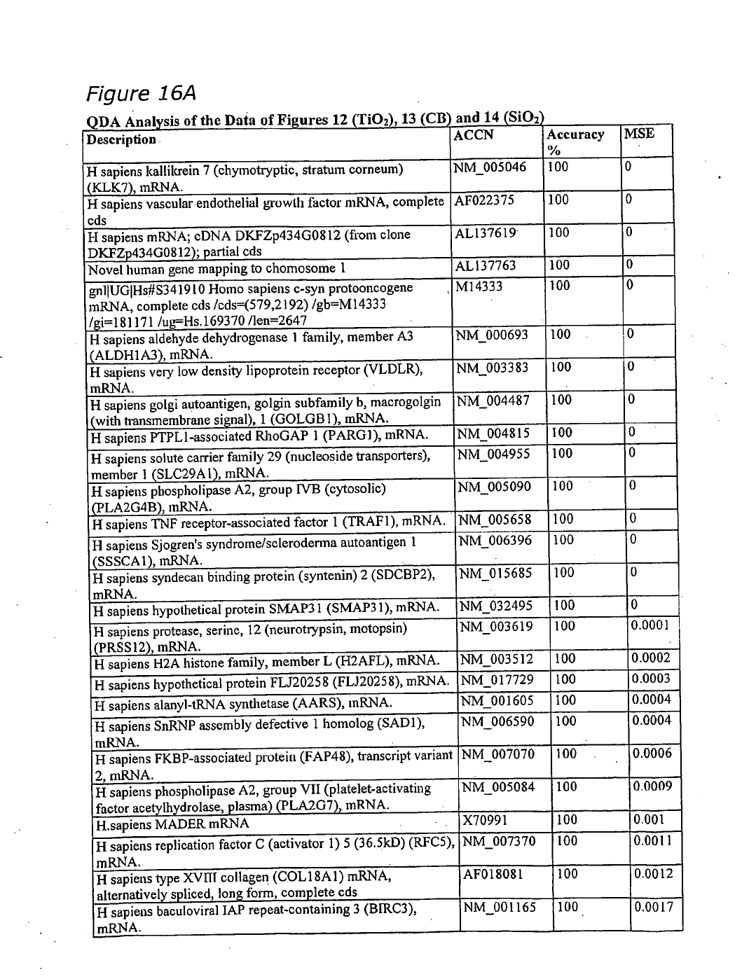

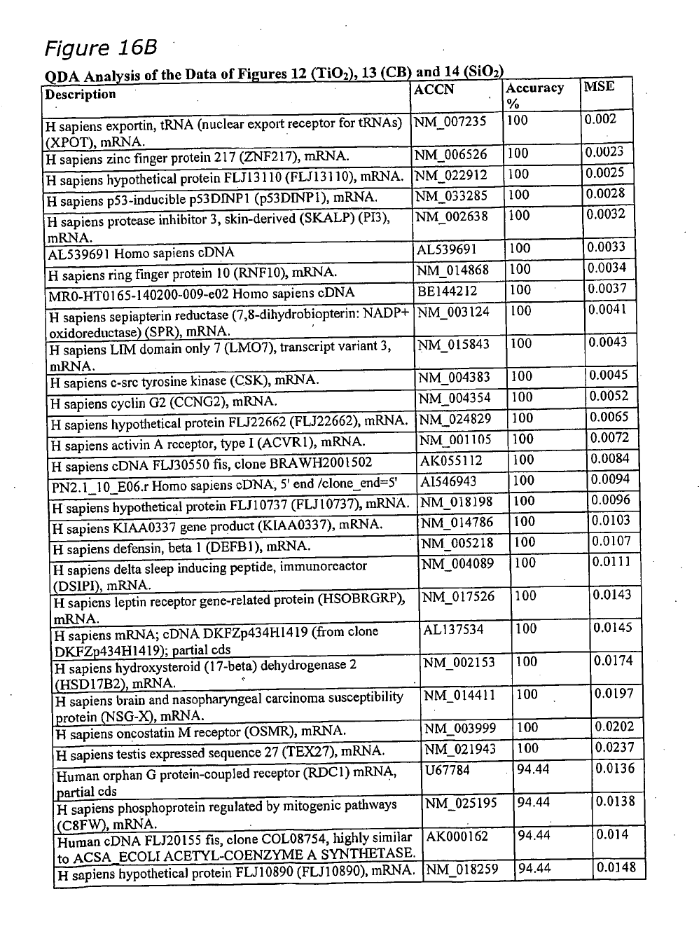

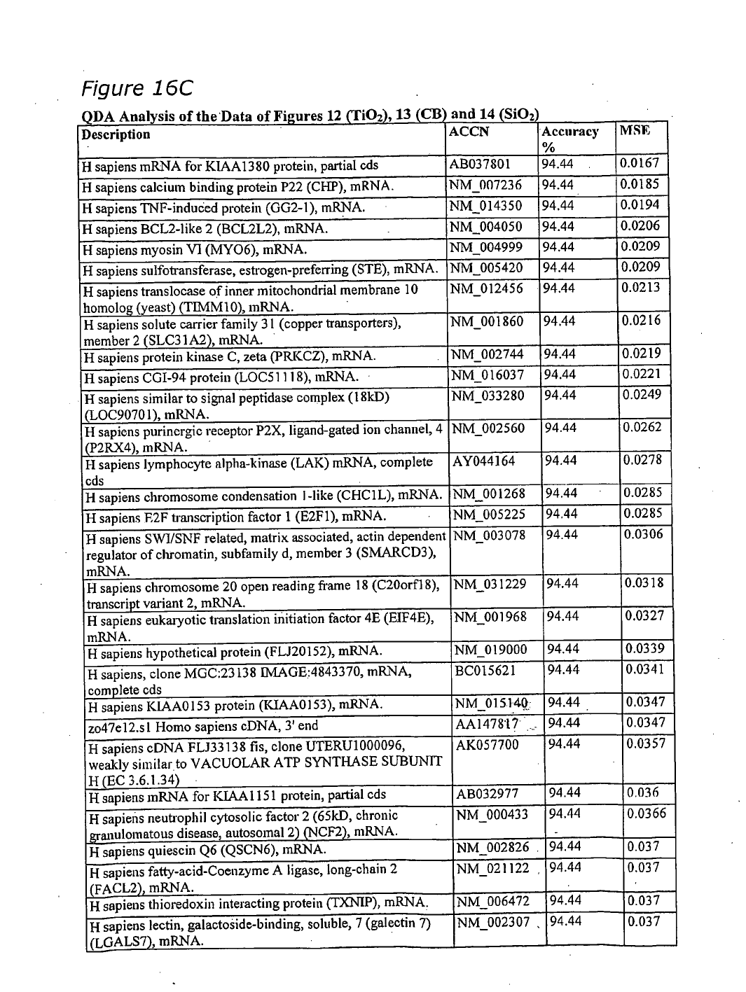

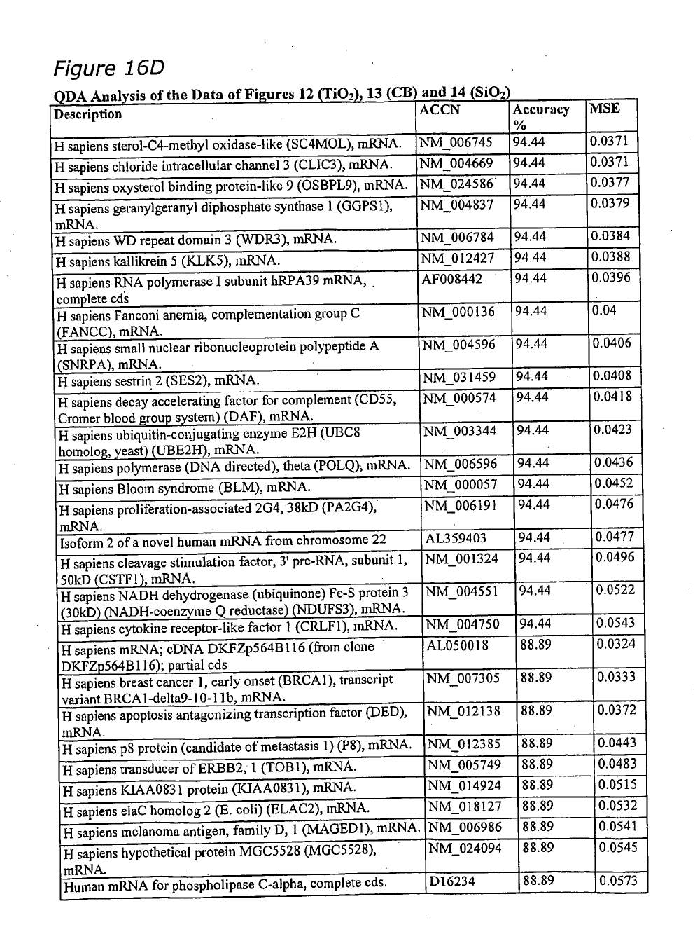

FIG. 14A1-4 presents GEM results for genes predominantly down-regulated in response to SiO₂ nanoparticle exposure in HEK cells.

FIG. 14B1-7 presents GEM results for genes predominantly up-regulated in response to SiO₂ nanoparticle exposure in HEK cells.

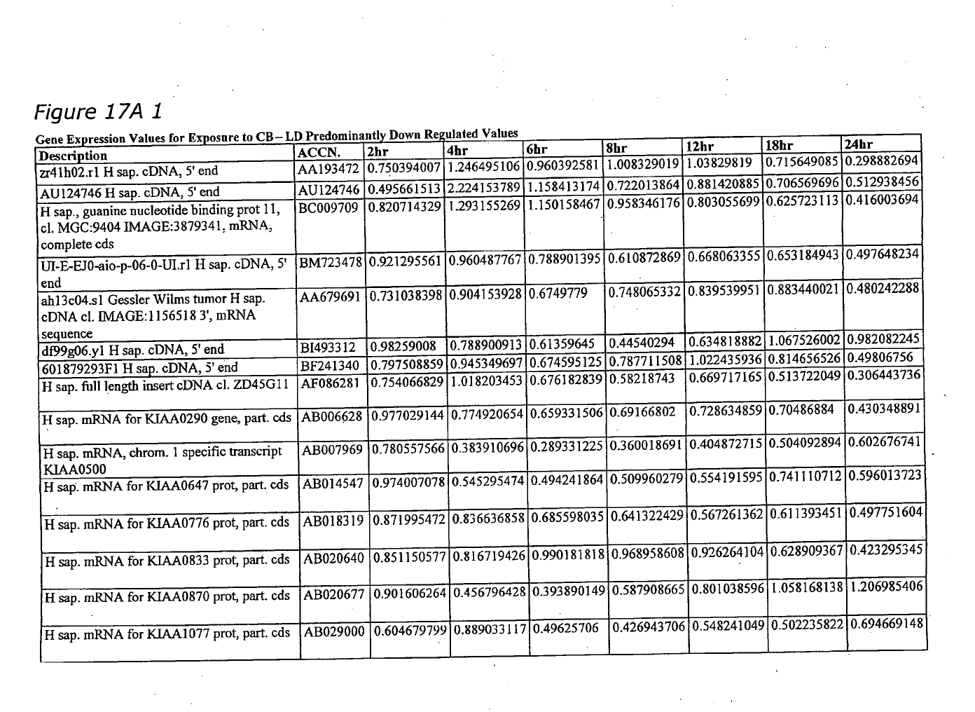

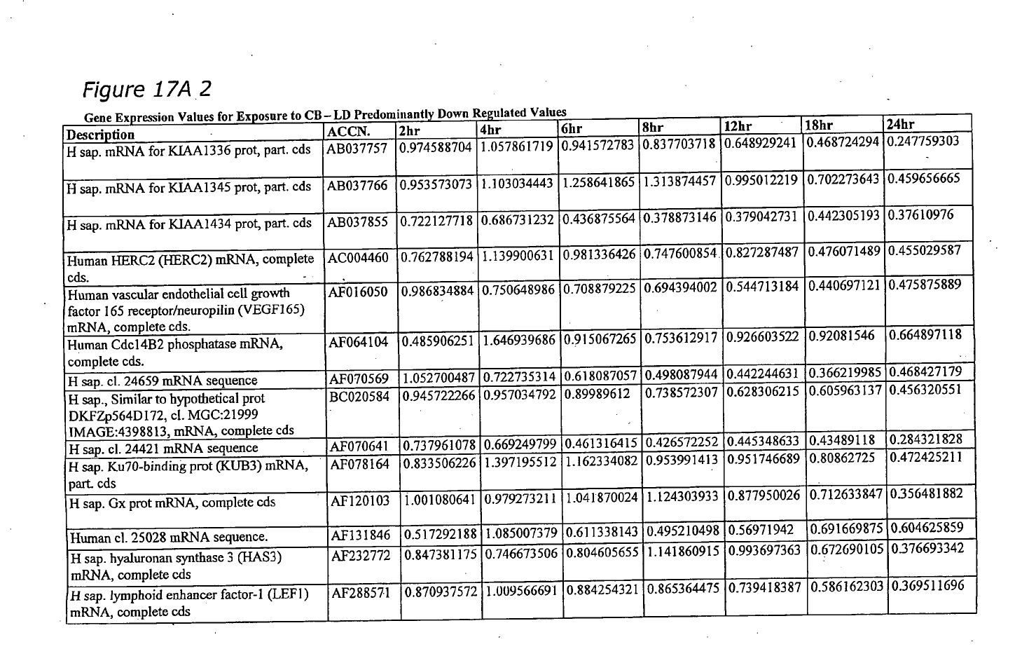

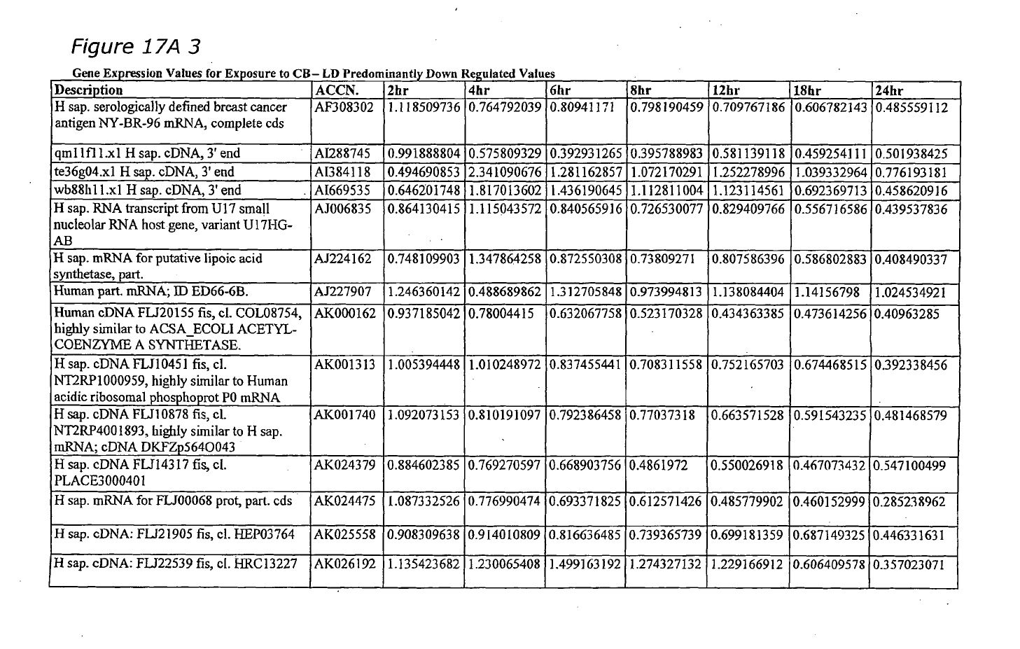

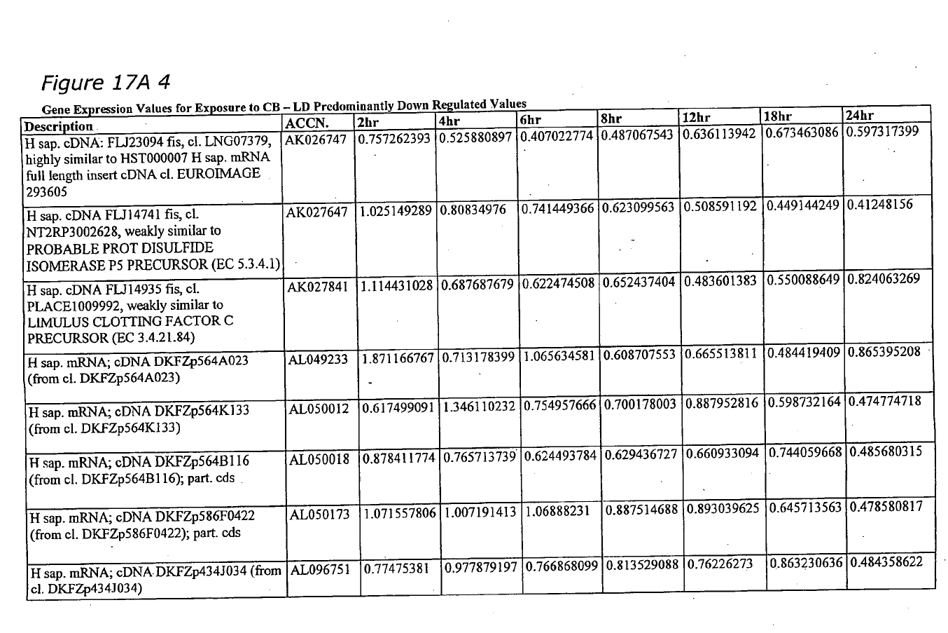

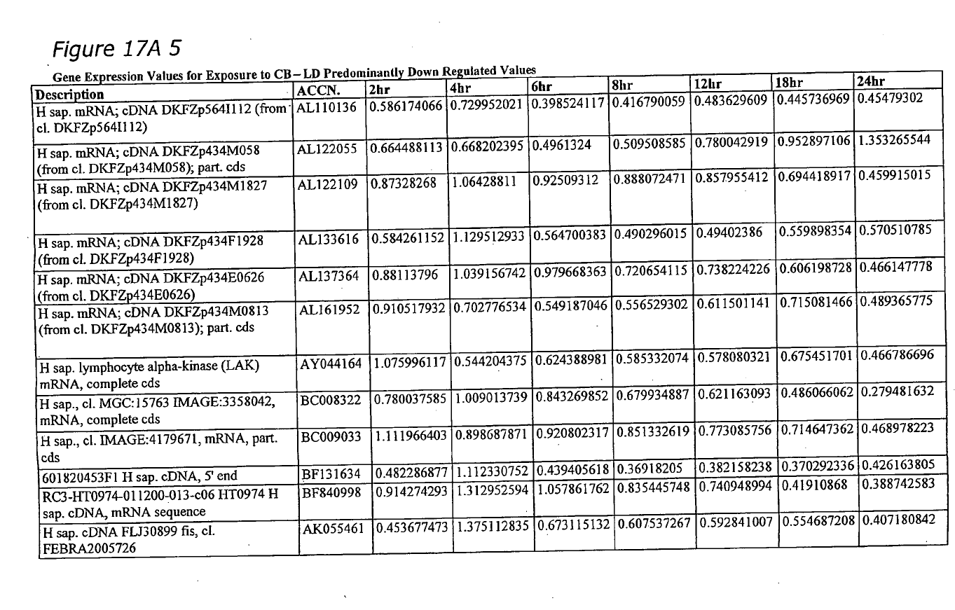

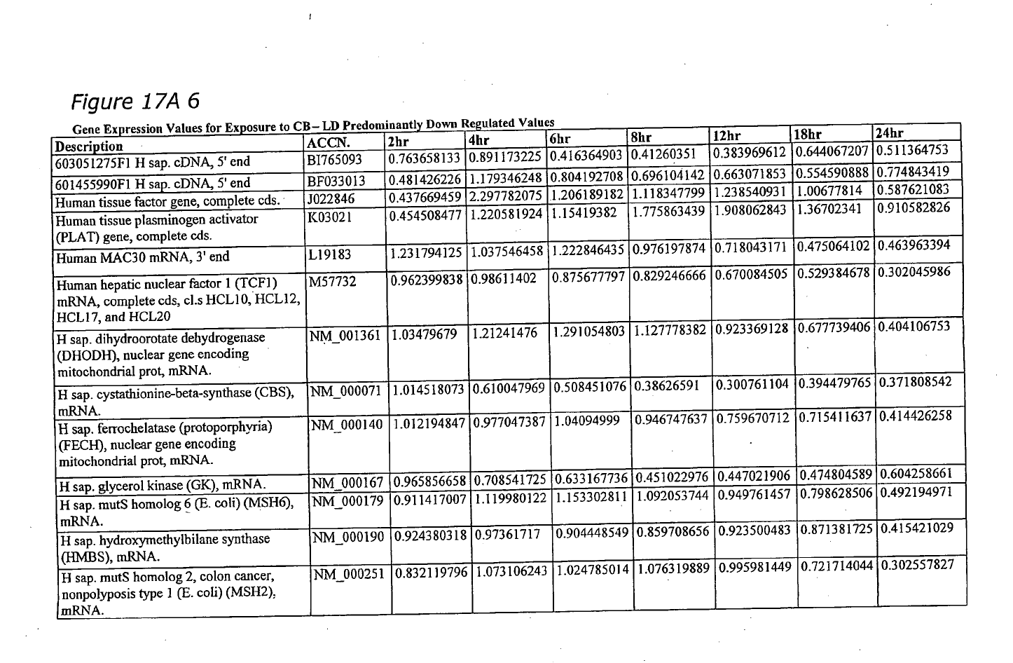

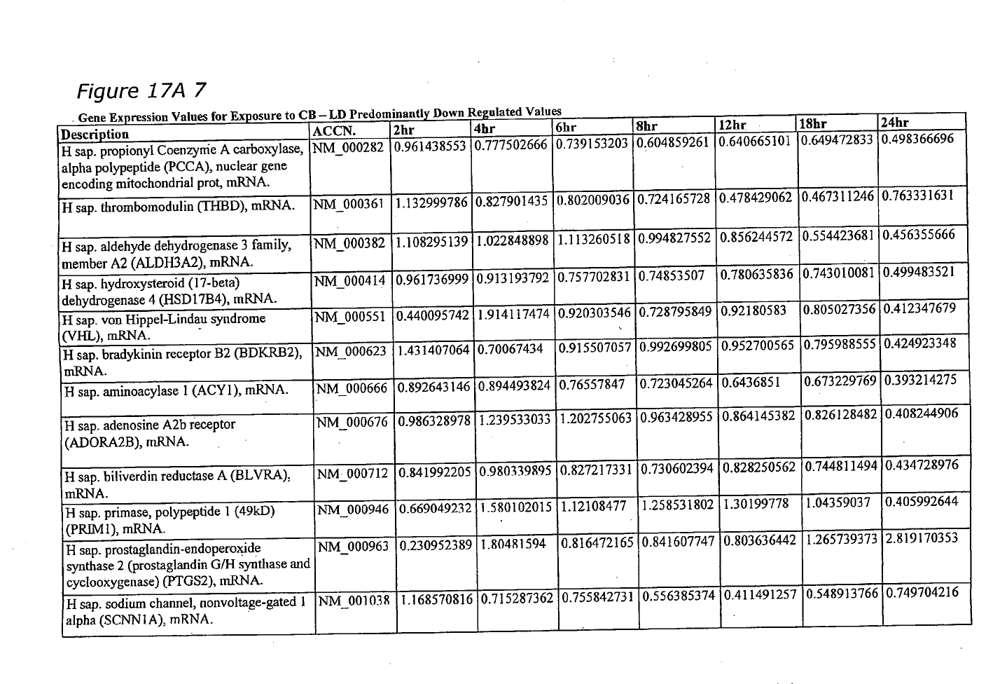

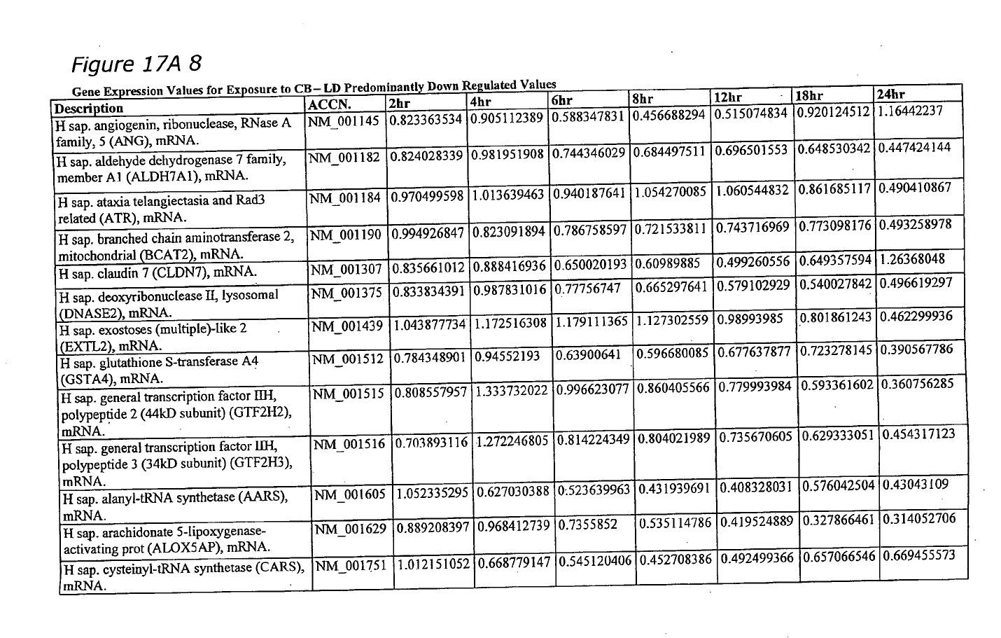

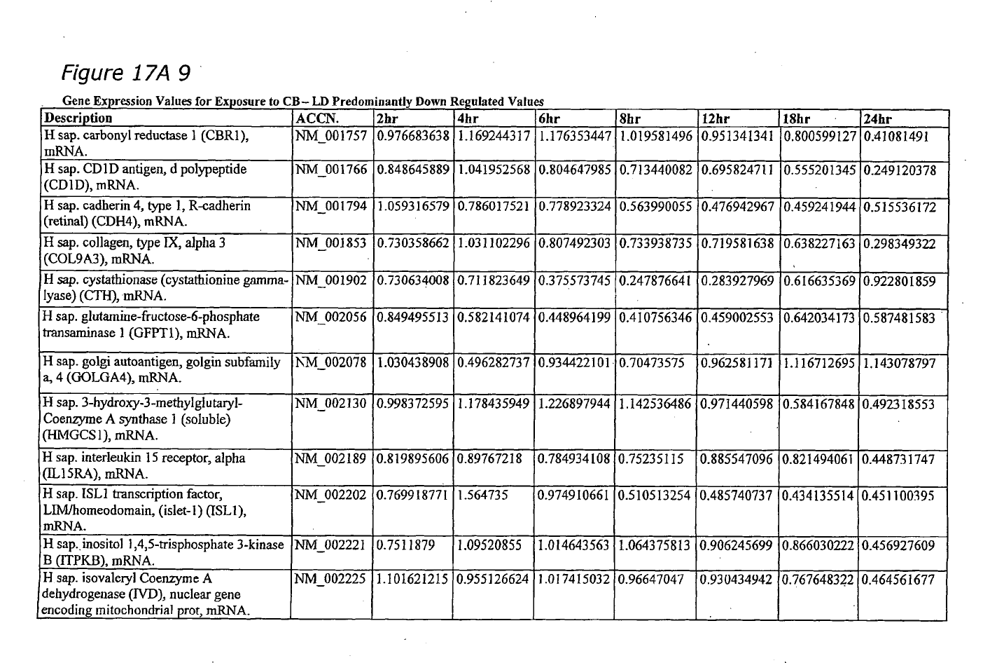

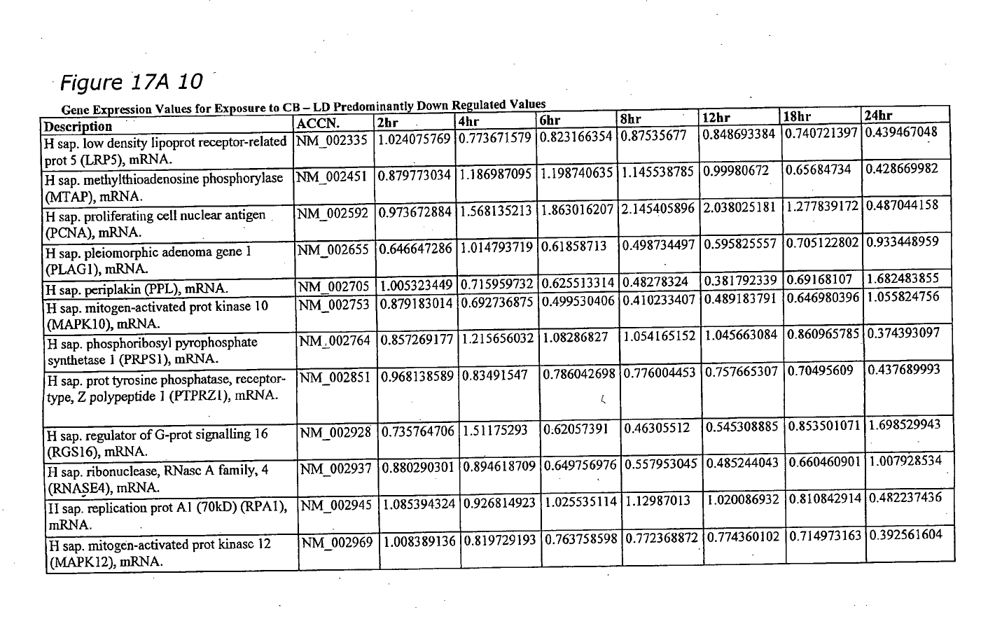

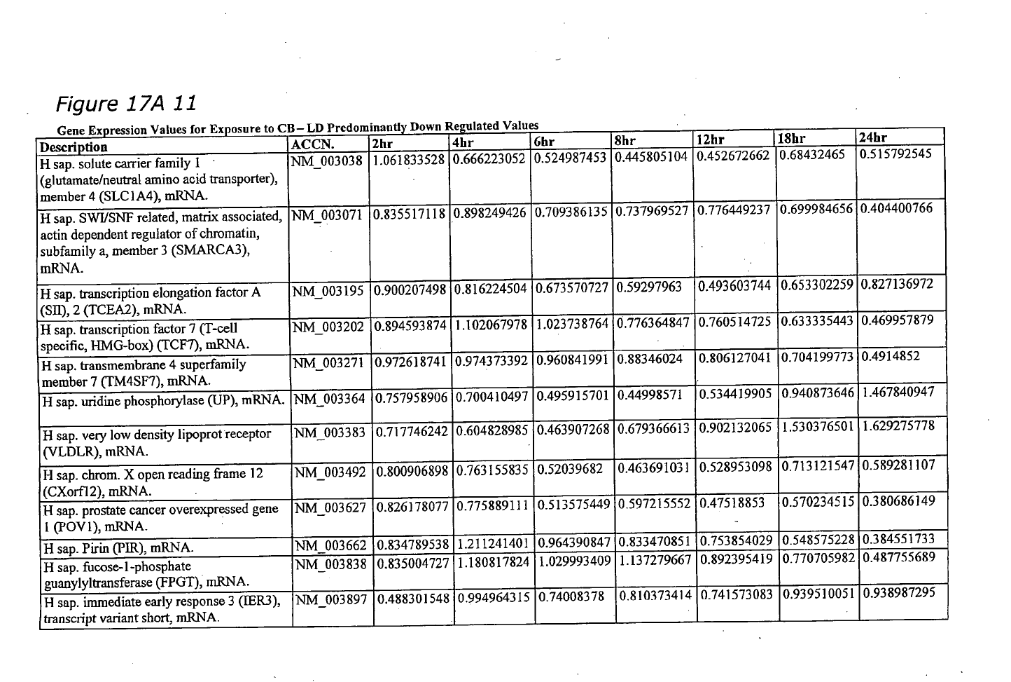

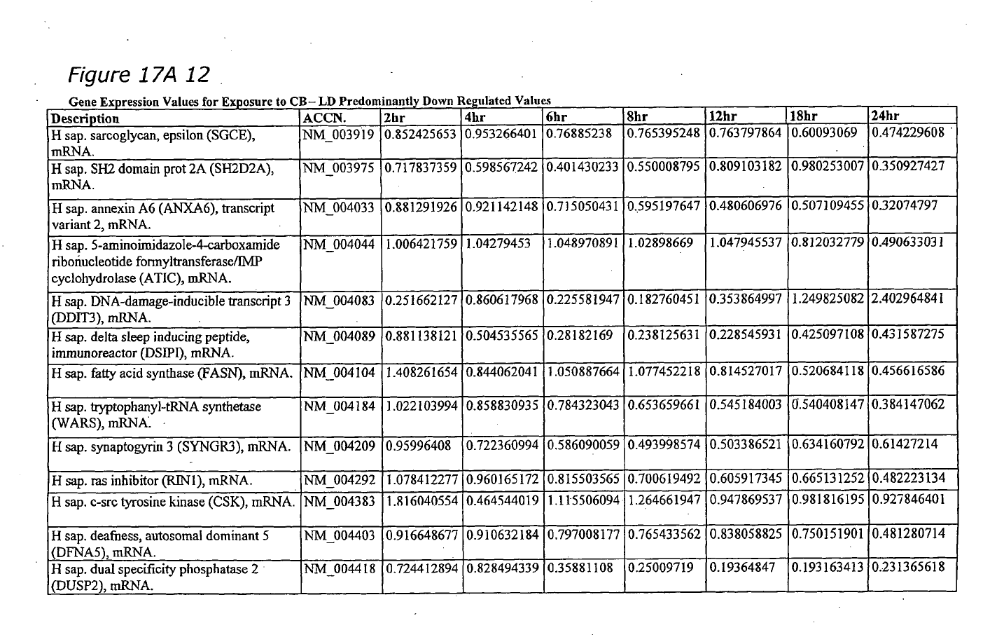

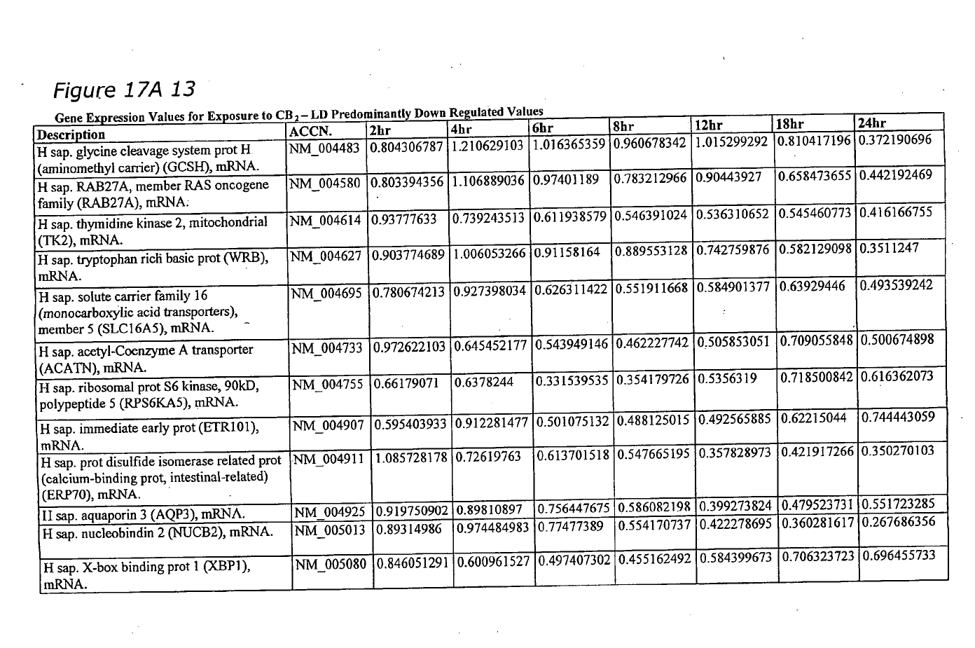

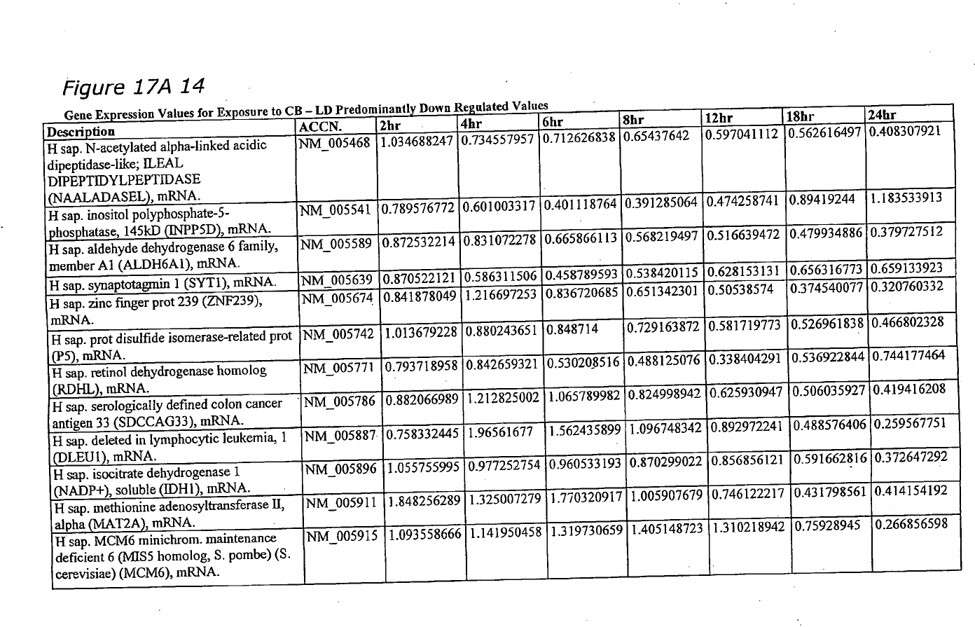

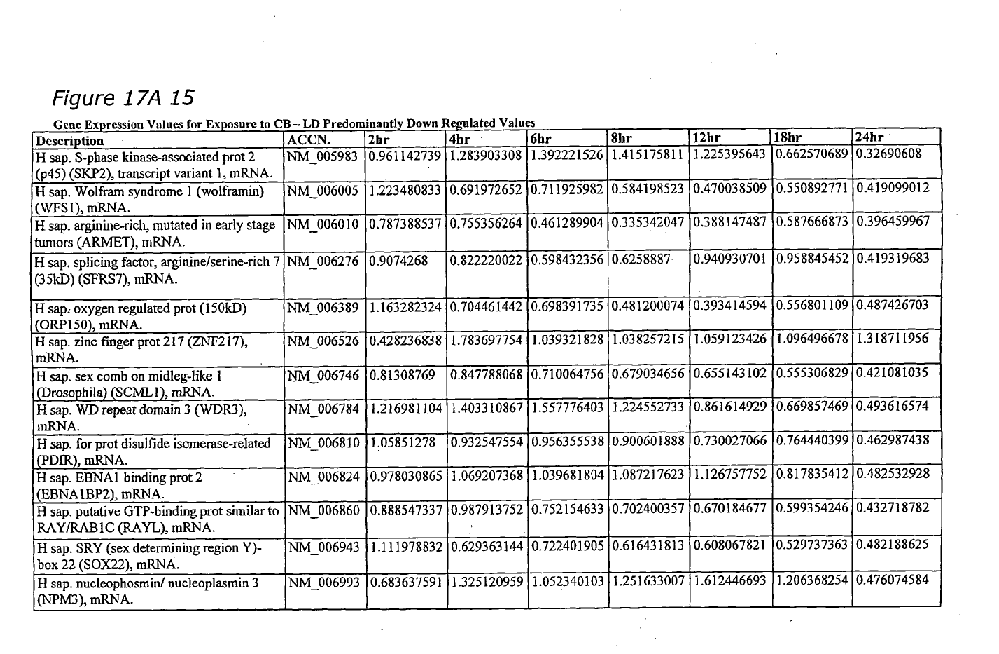

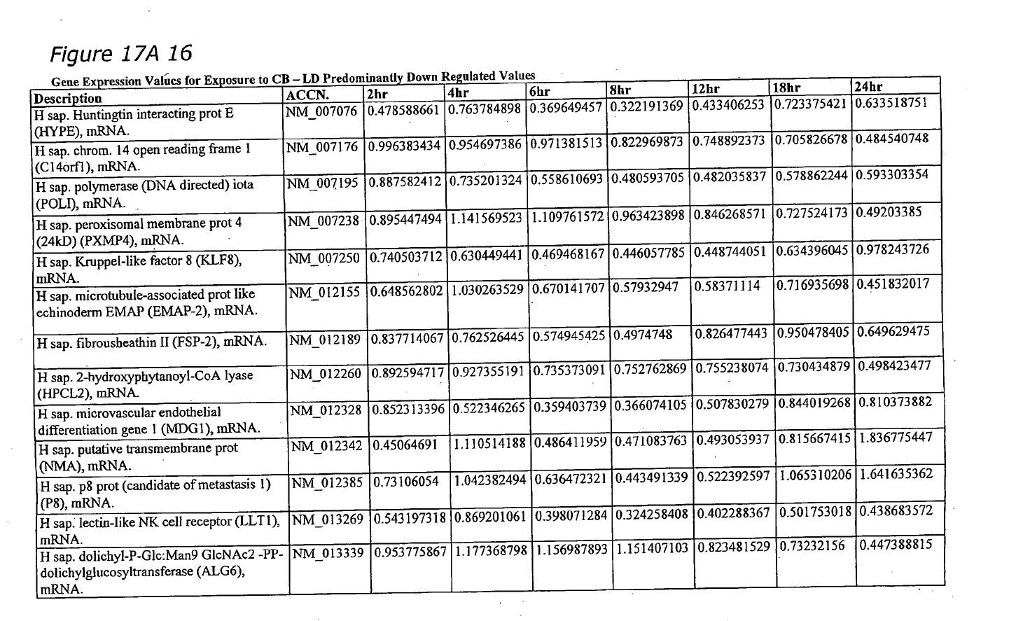

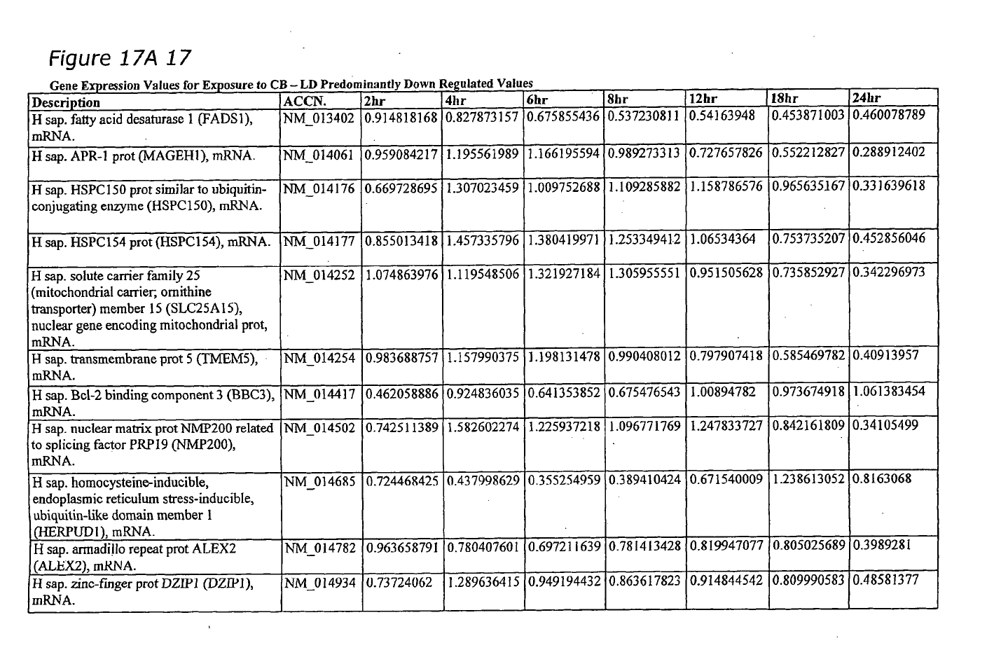

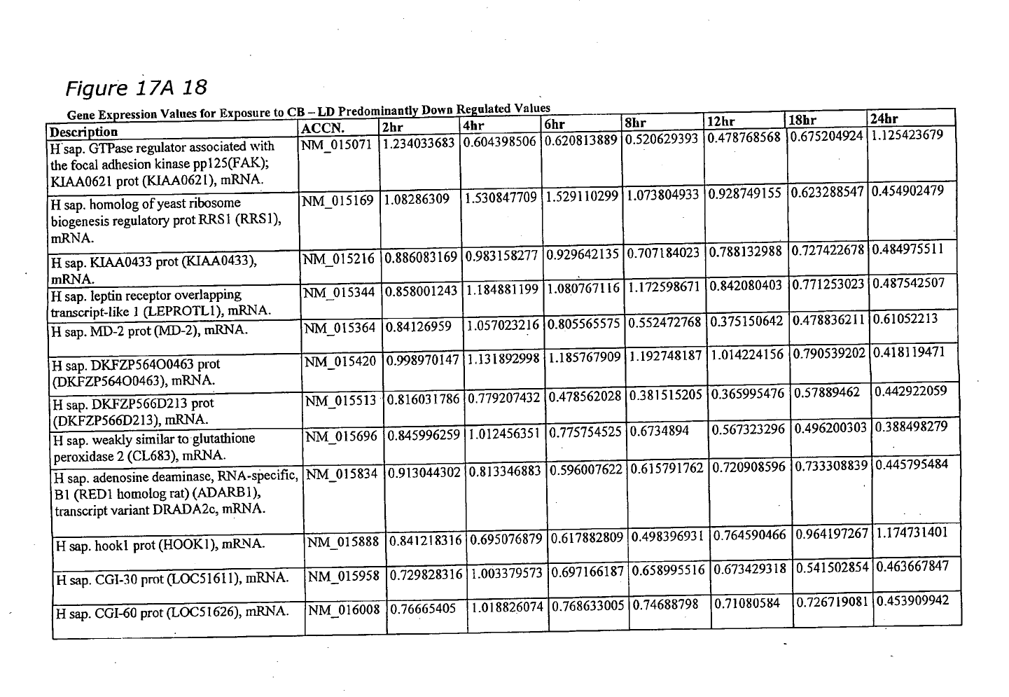

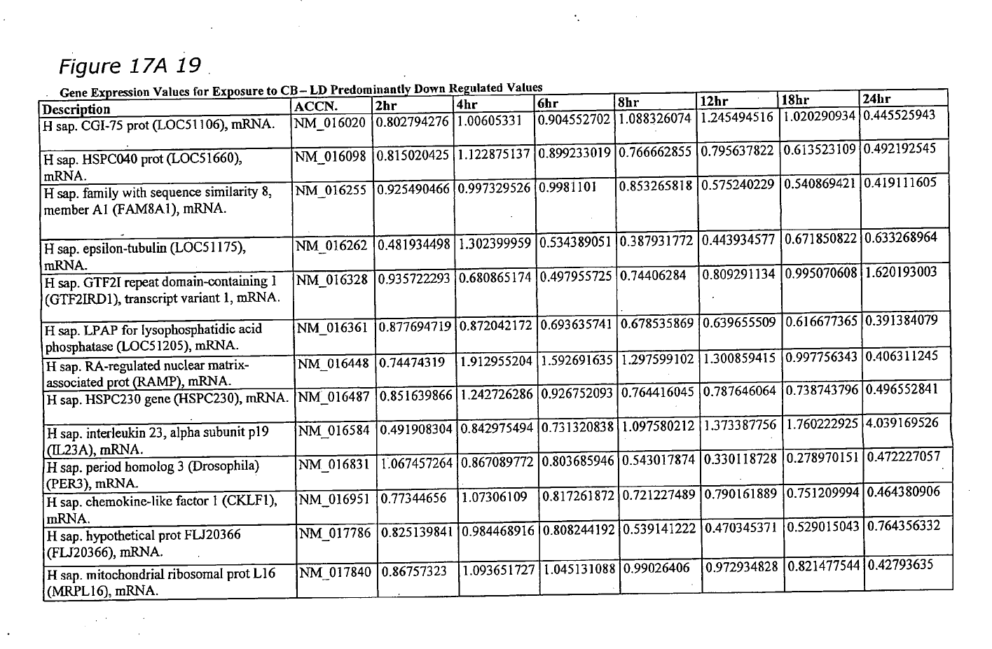

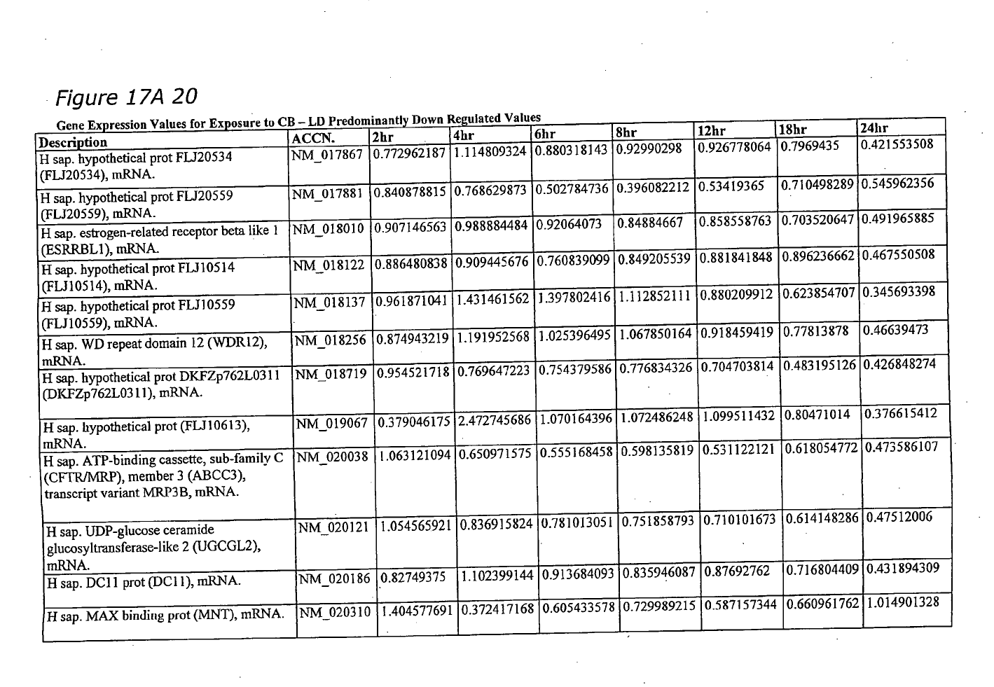

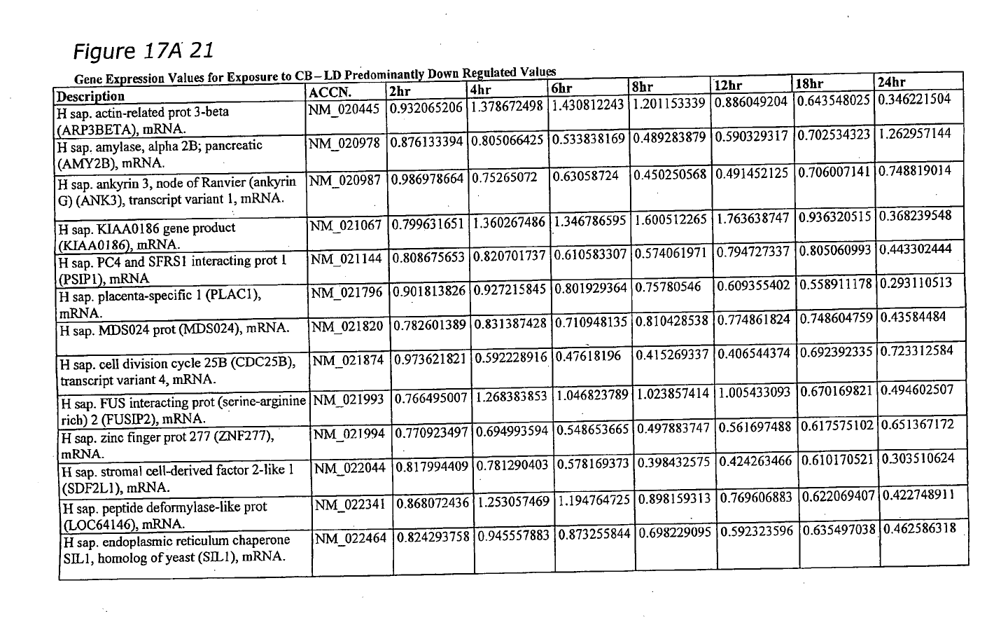

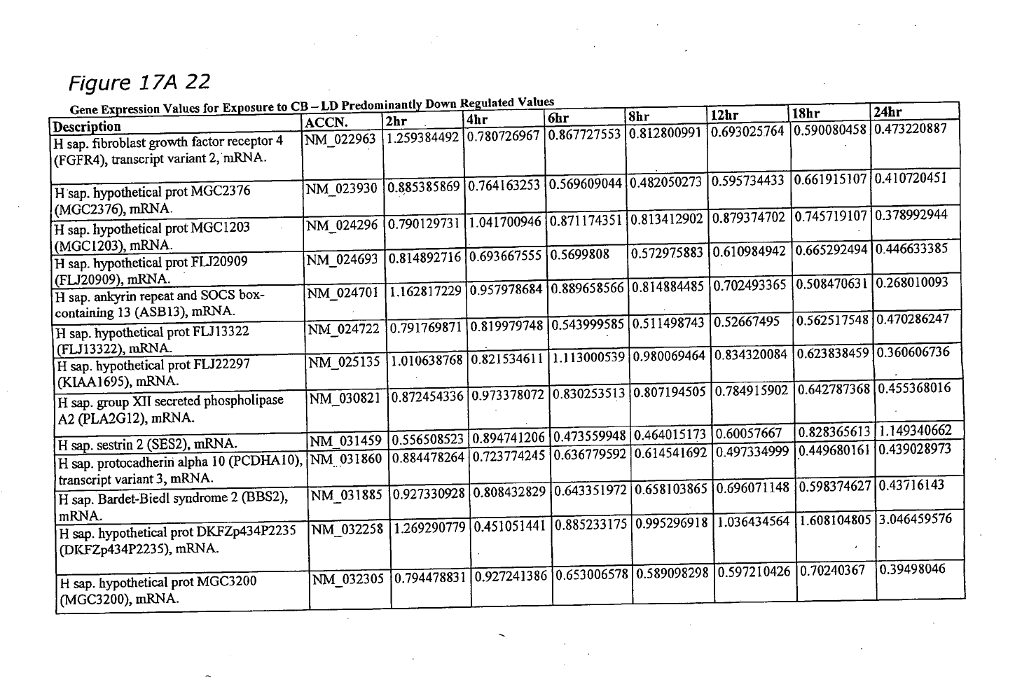

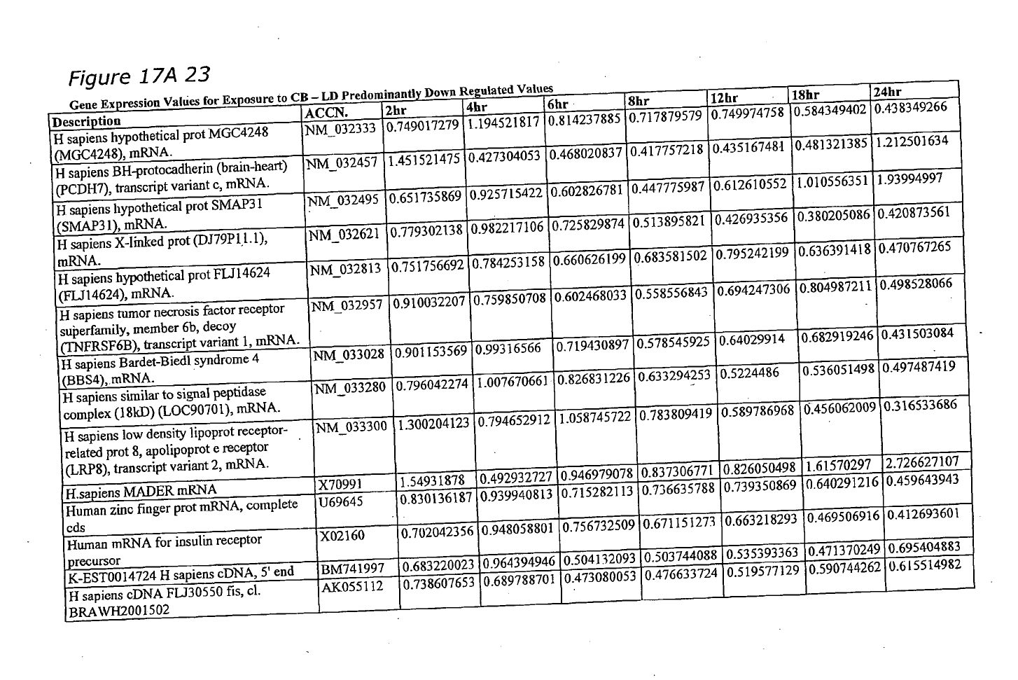

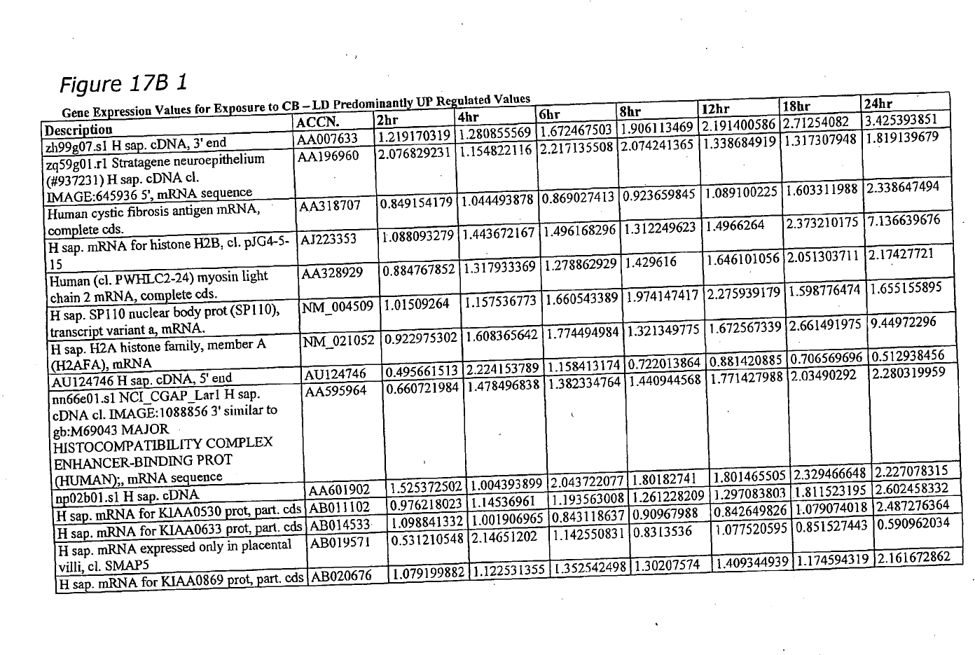

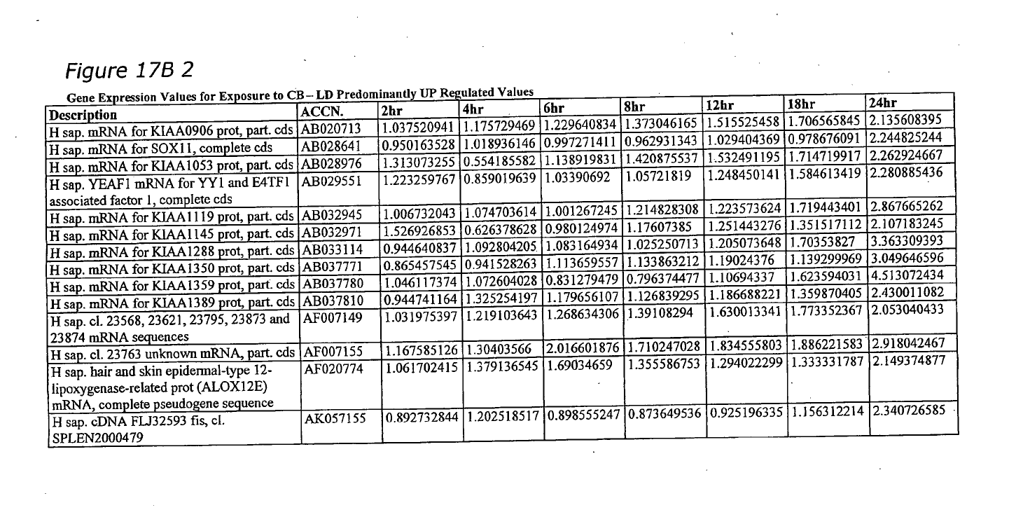

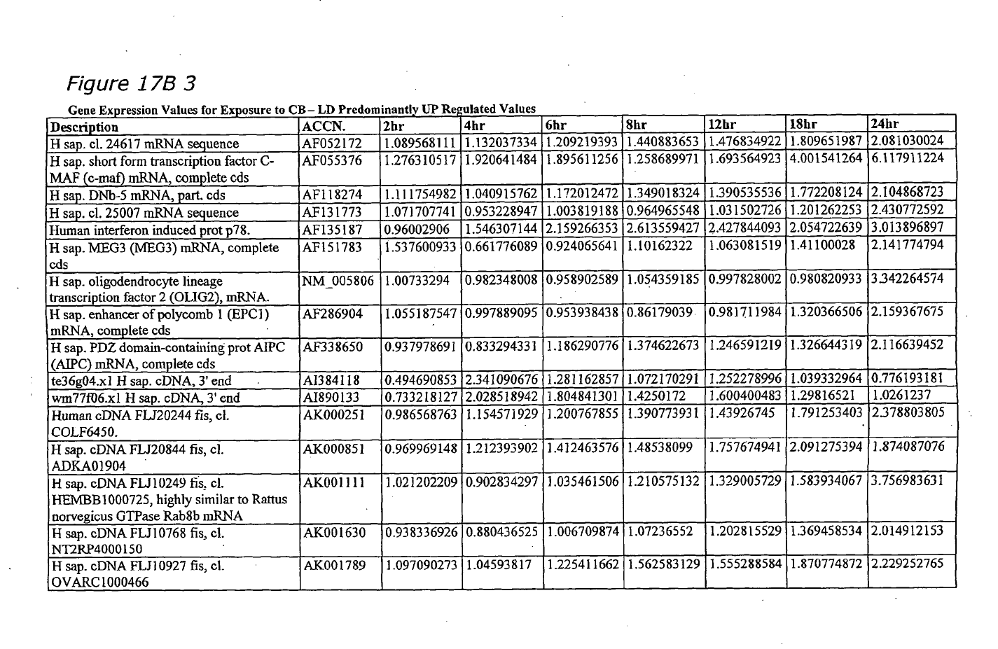

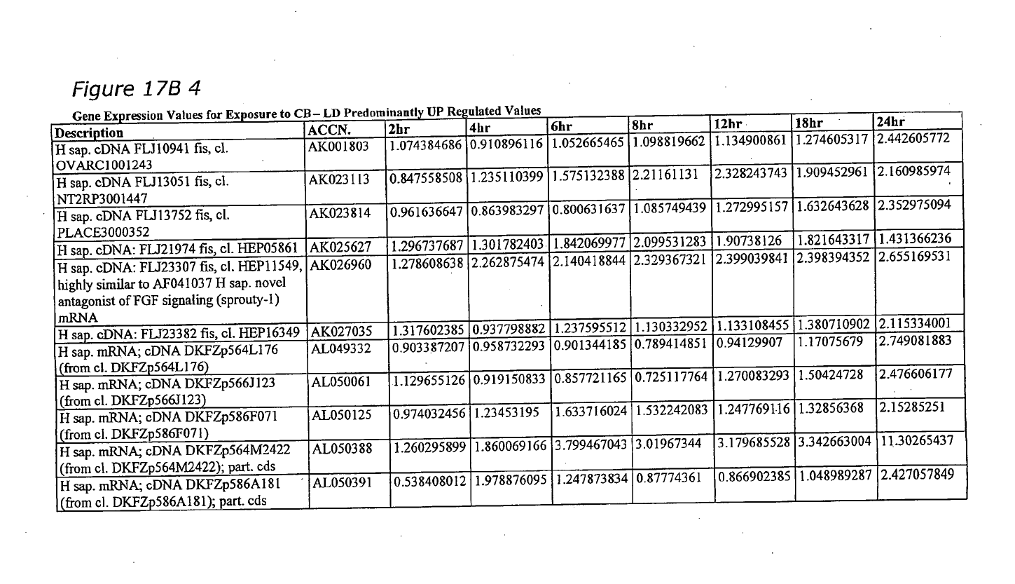

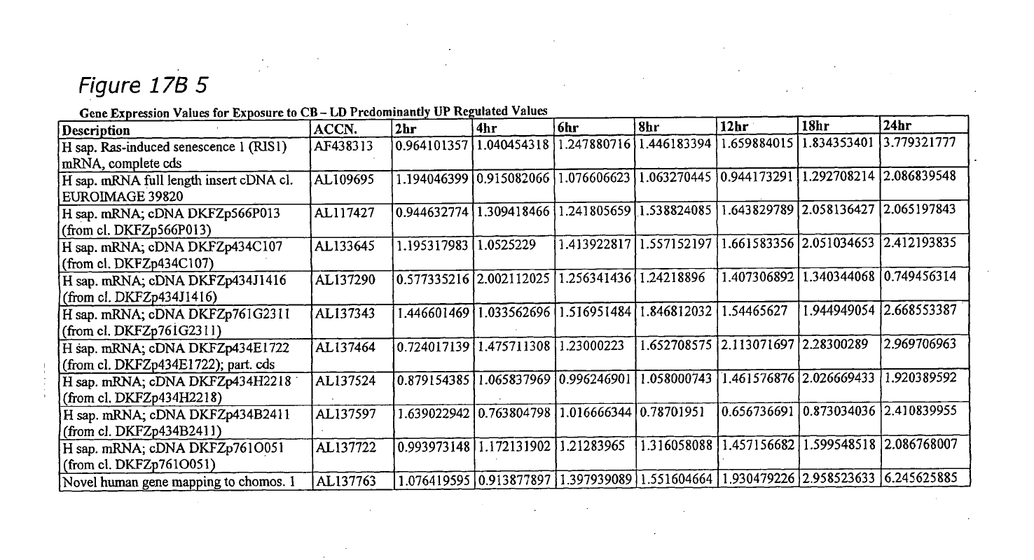

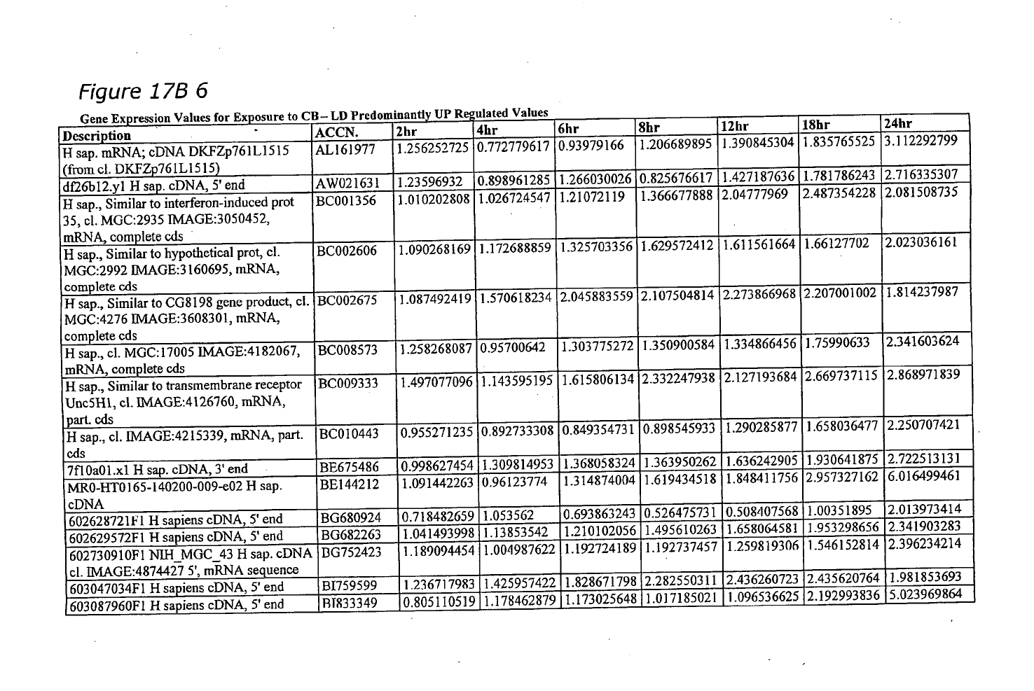

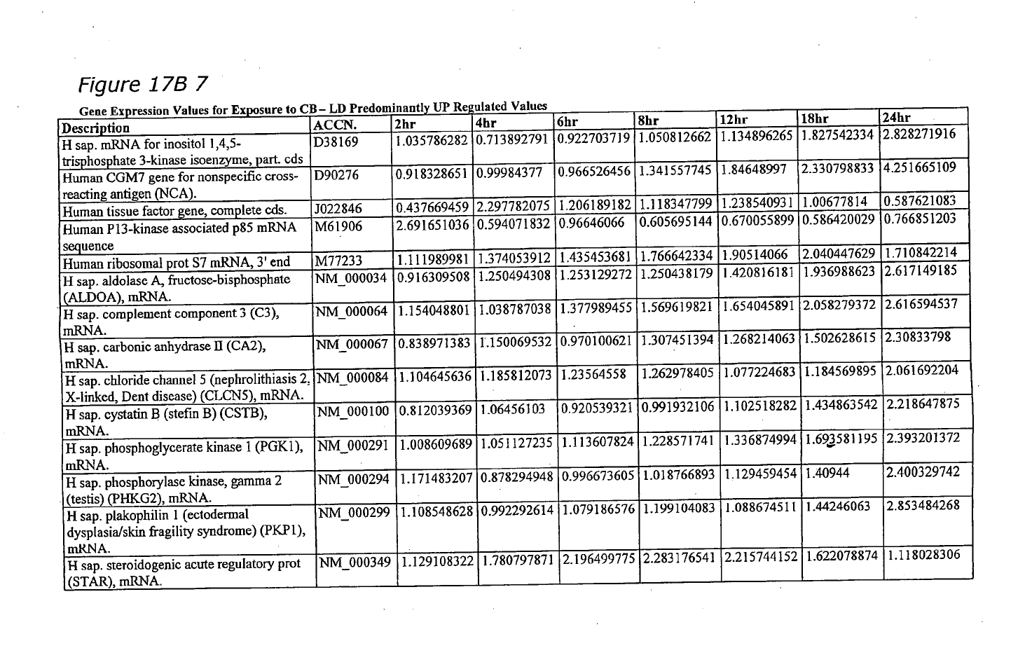

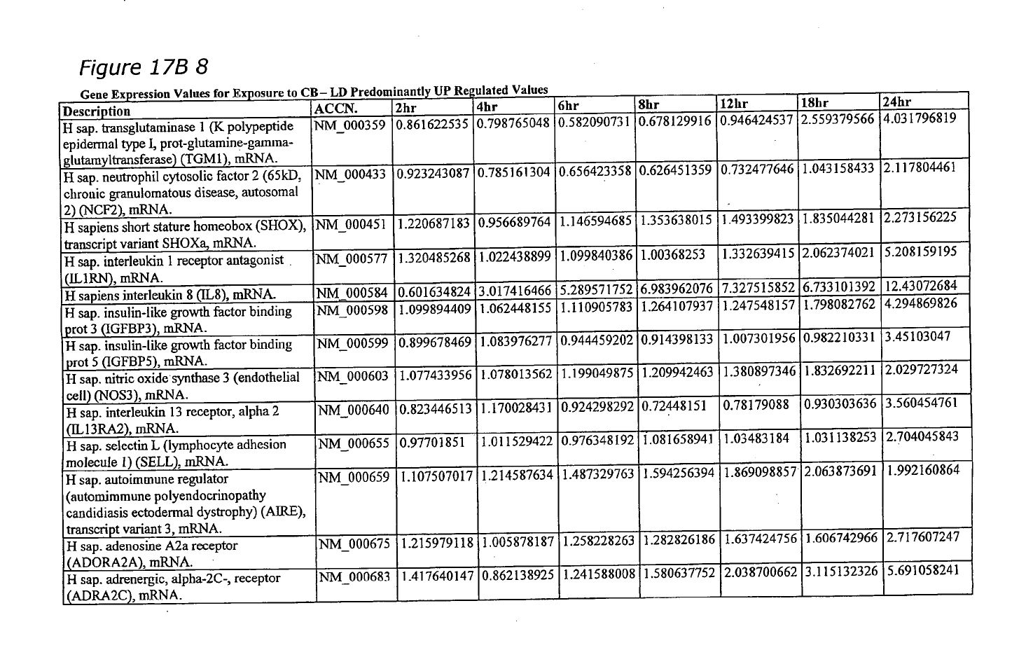

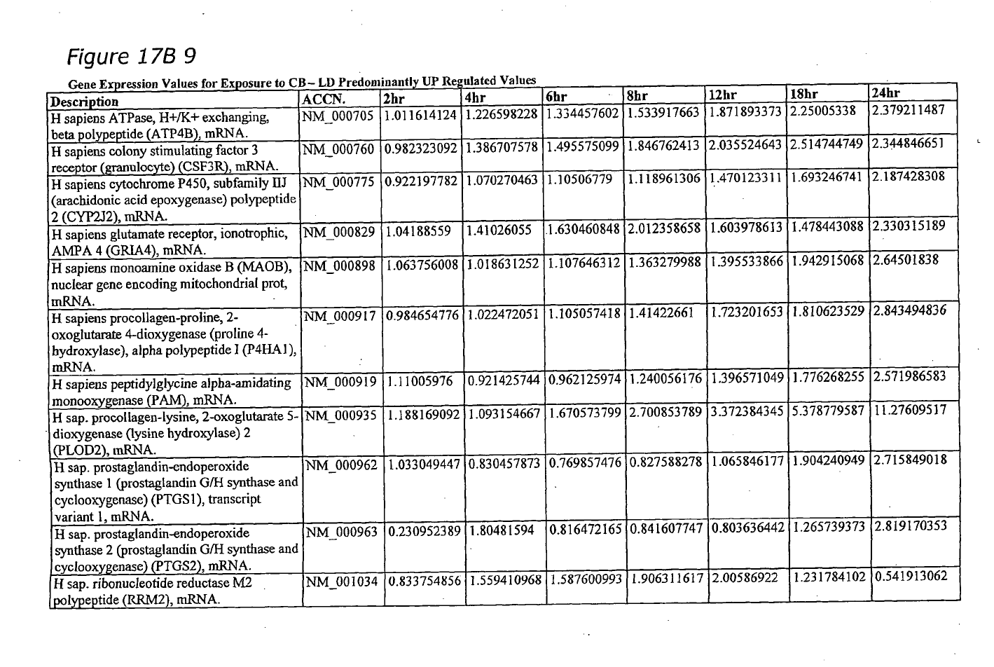

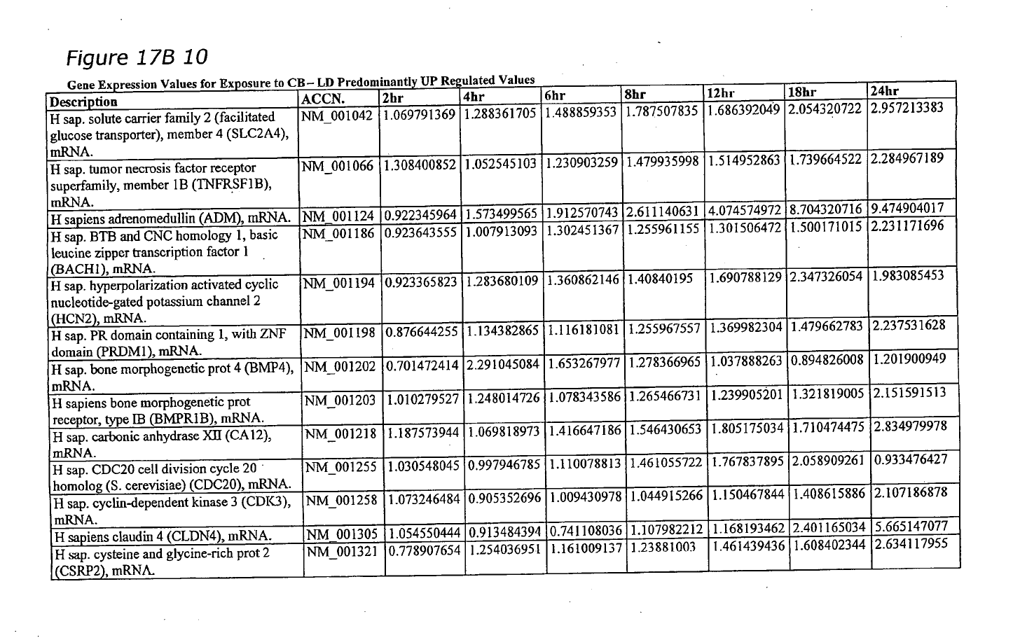

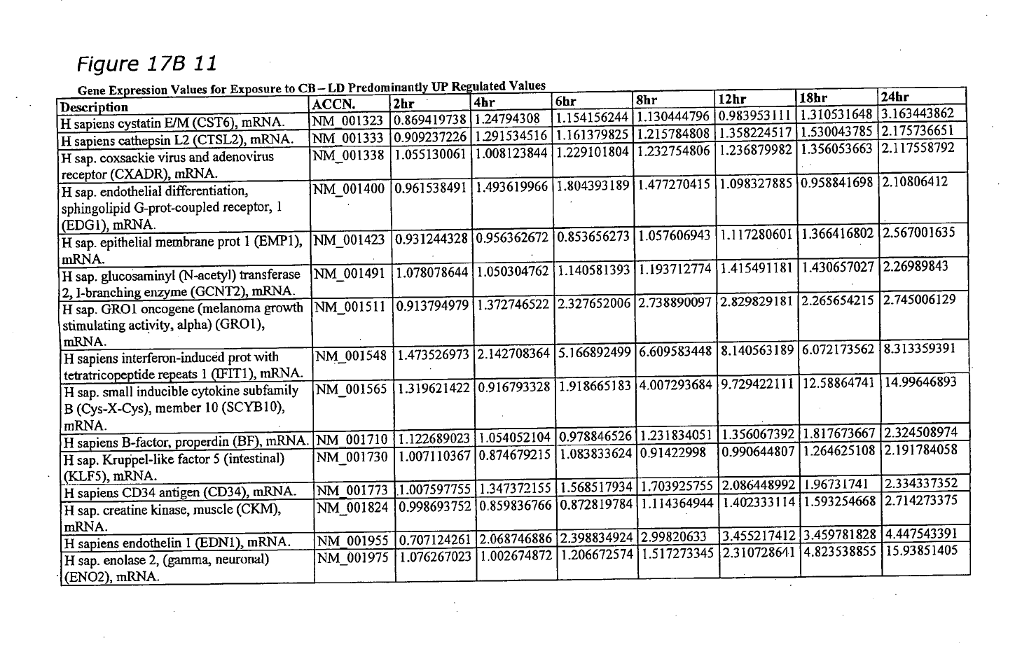

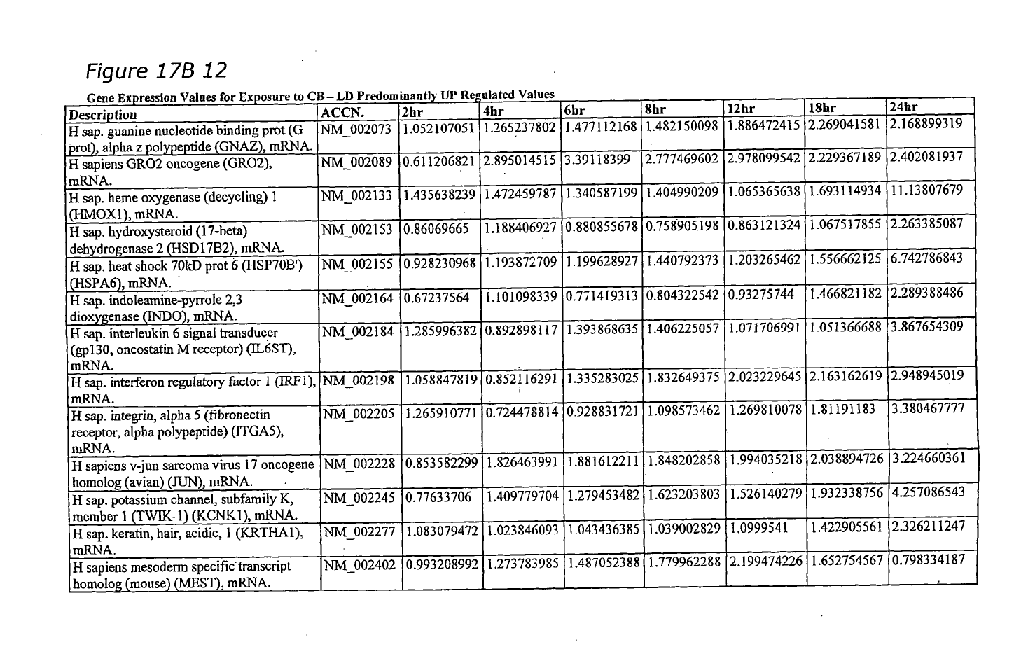

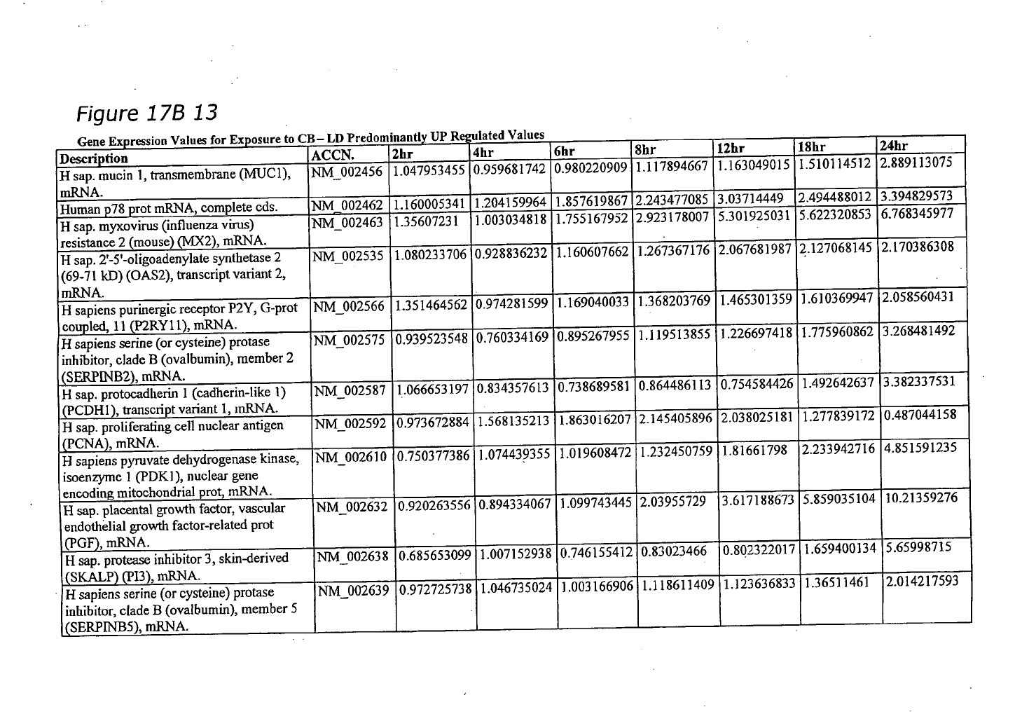

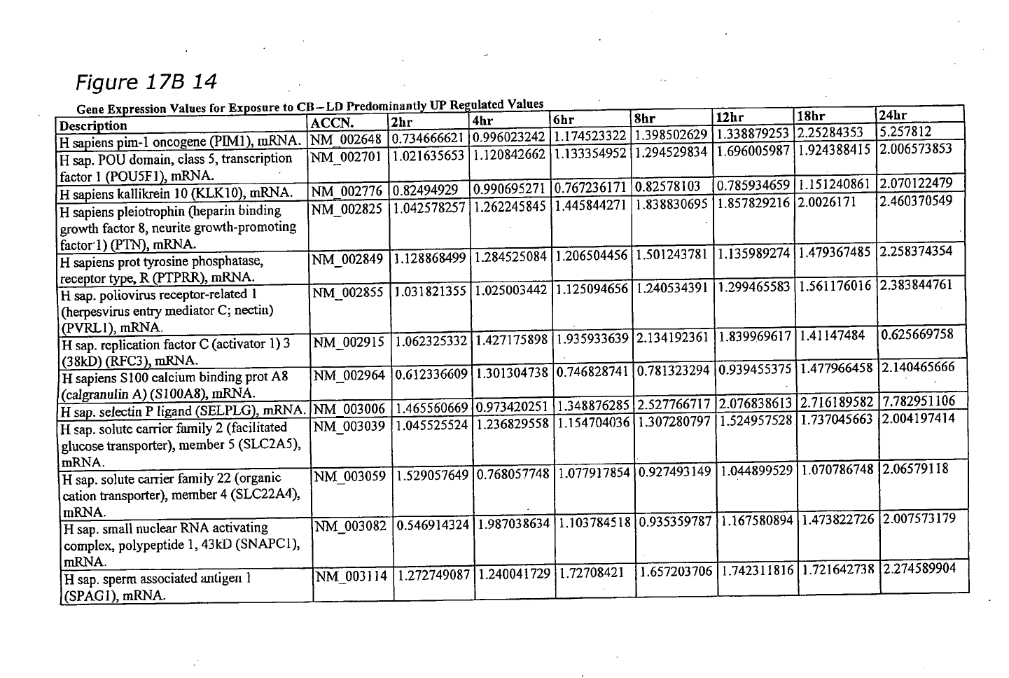

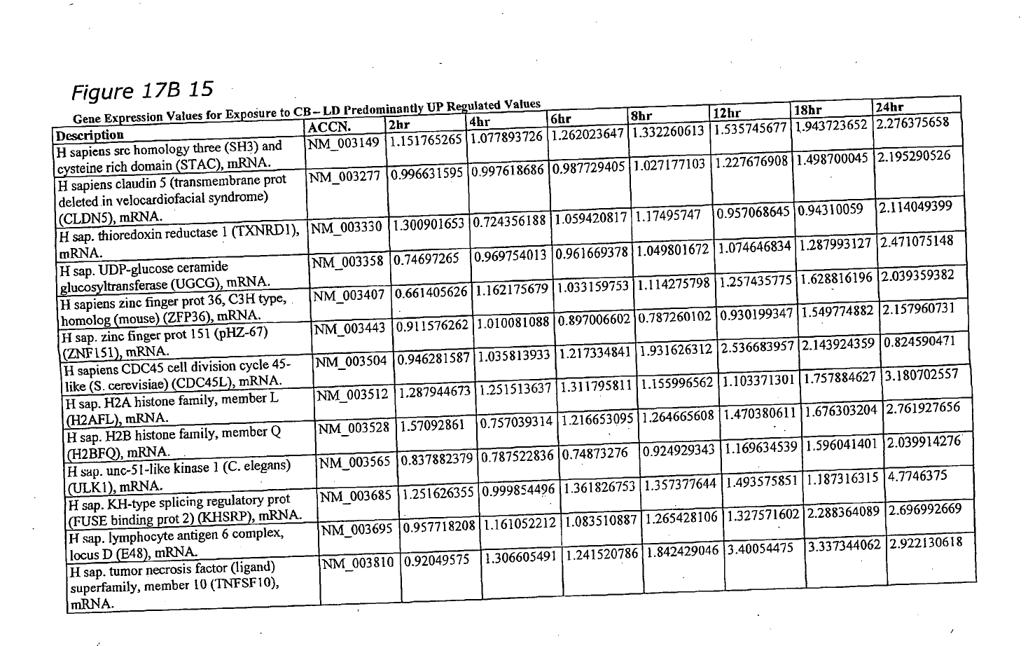

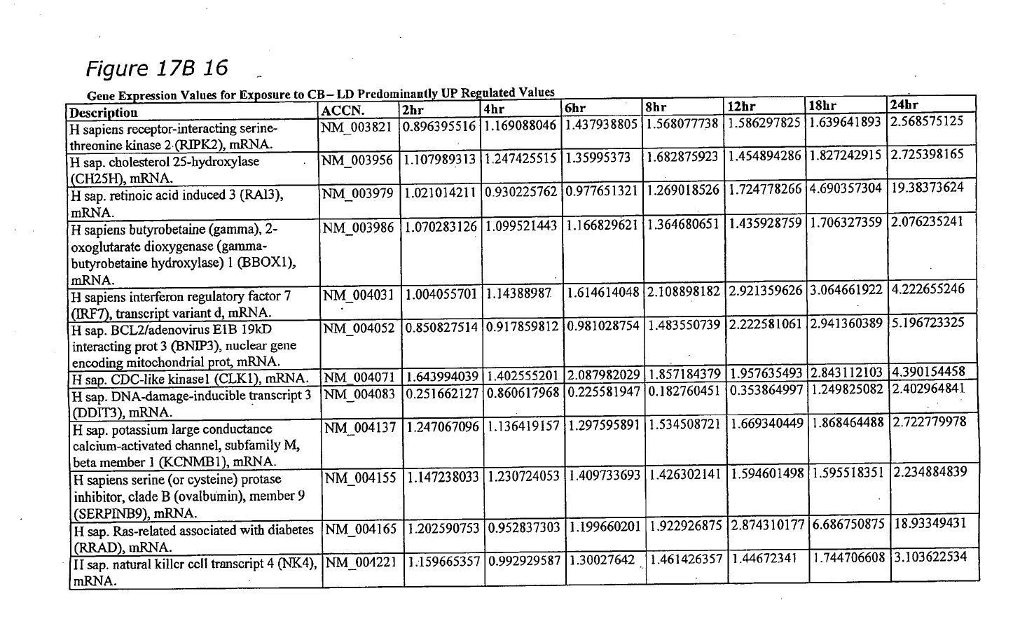

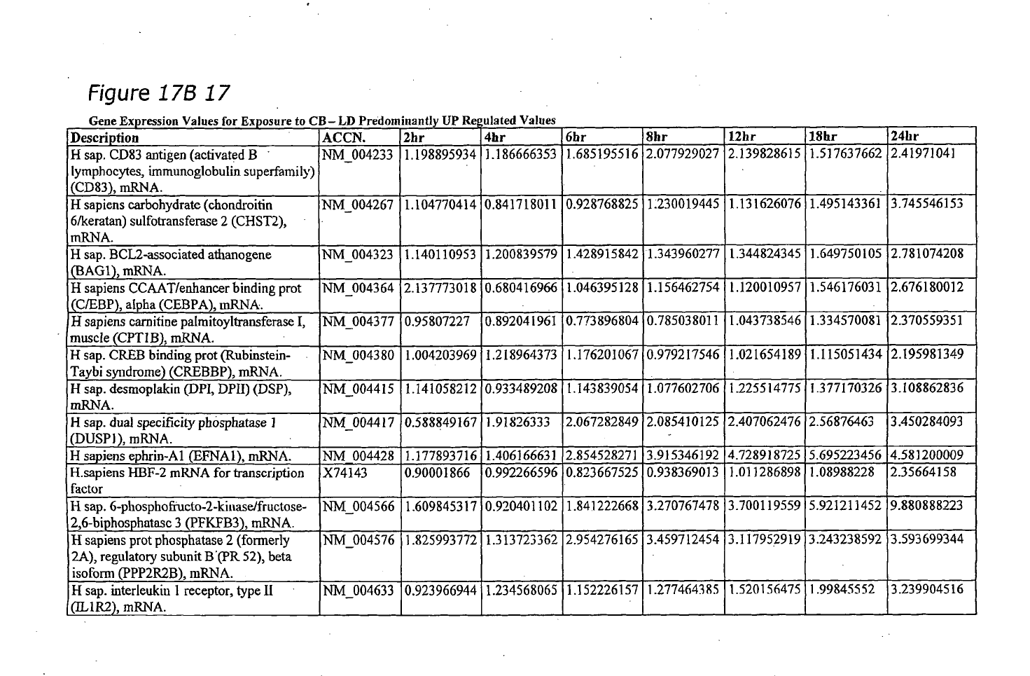

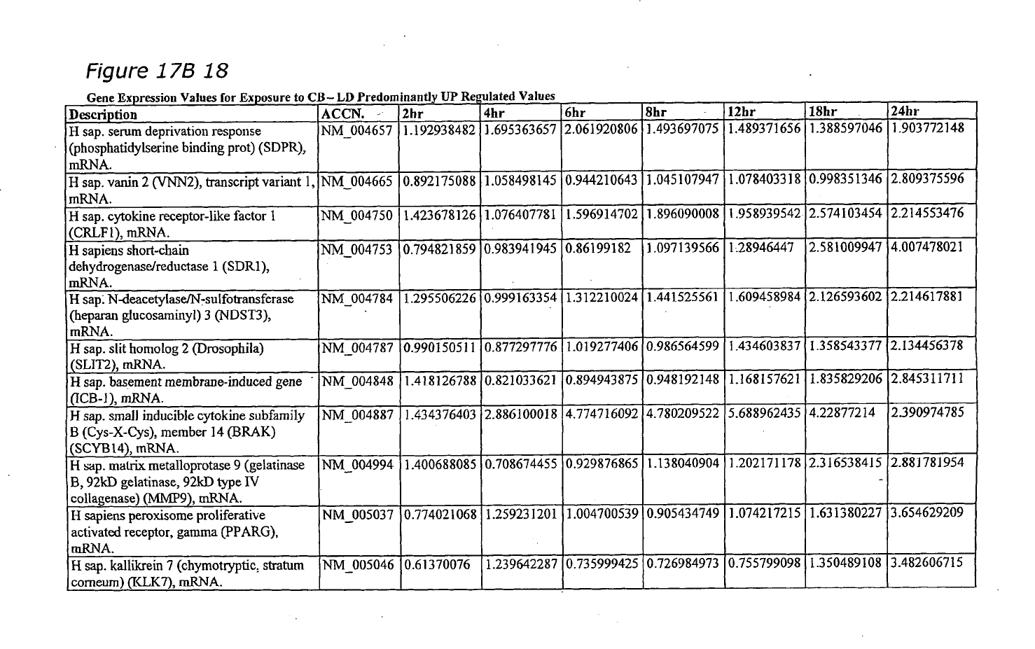

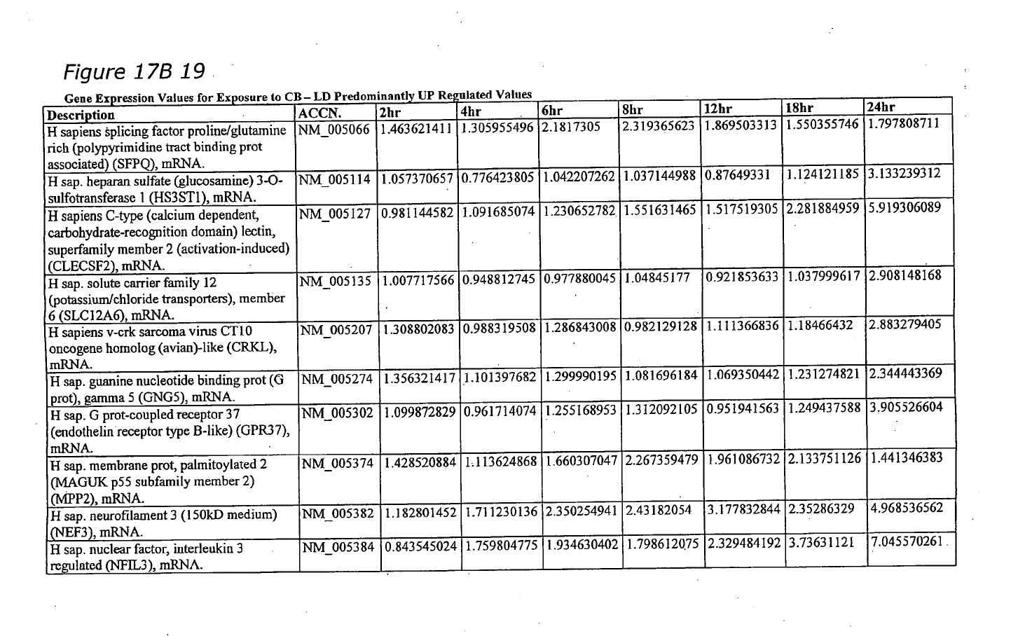

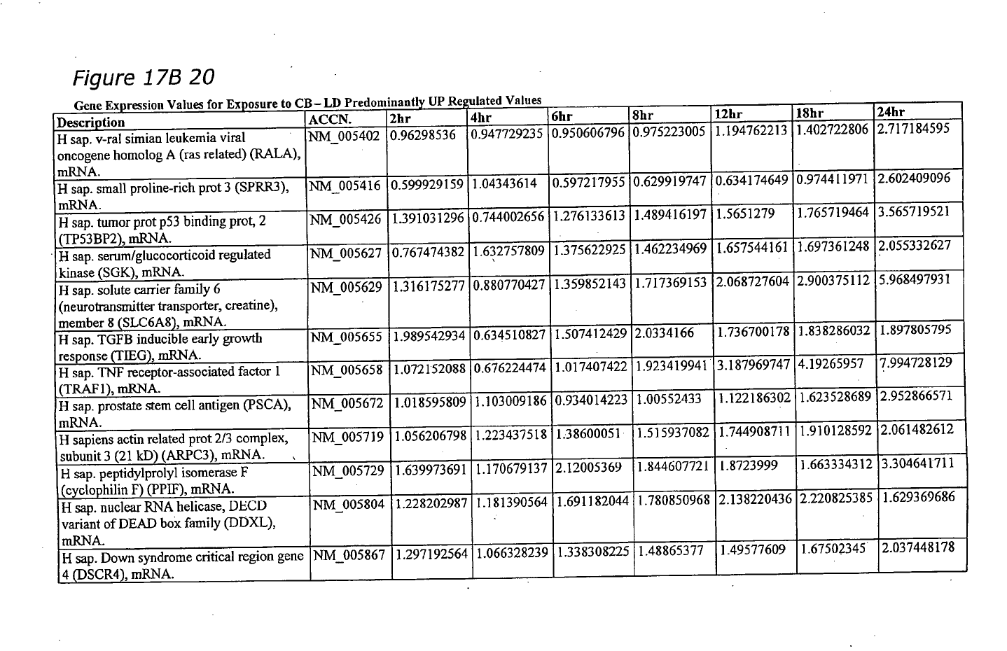

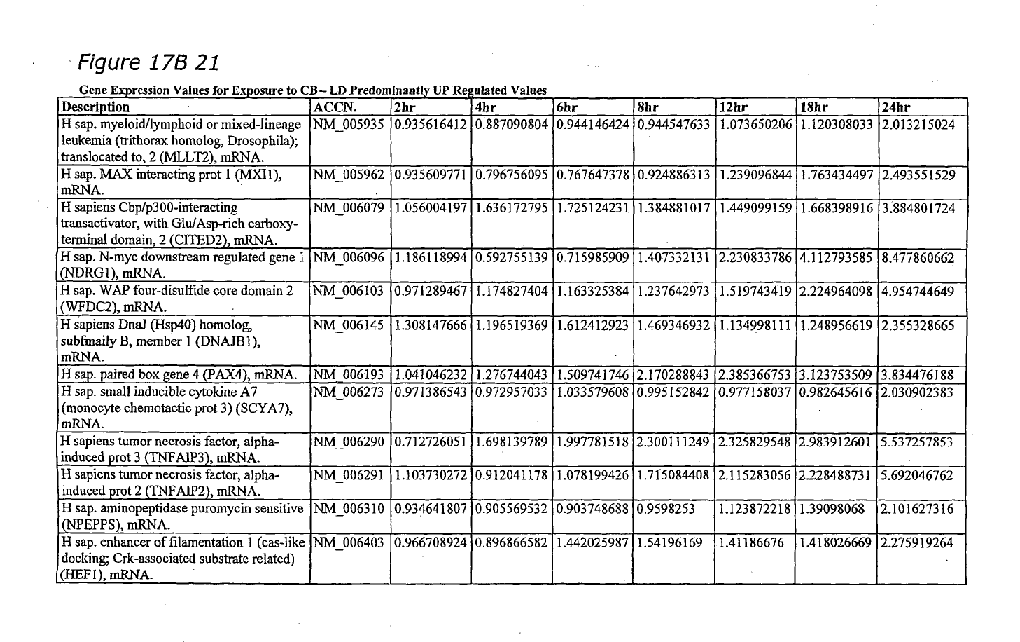

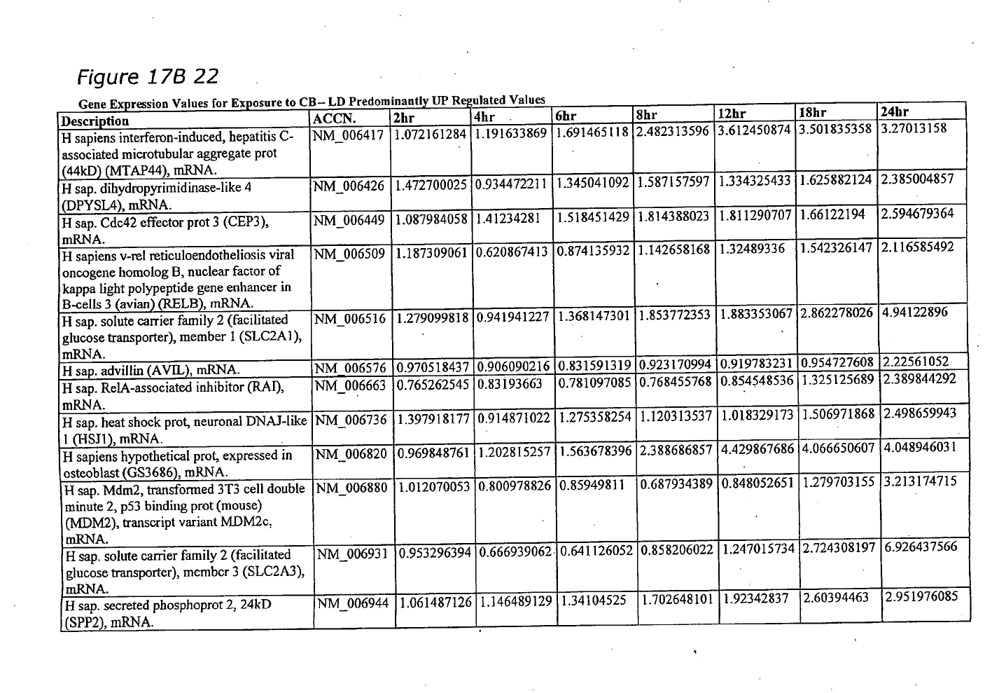

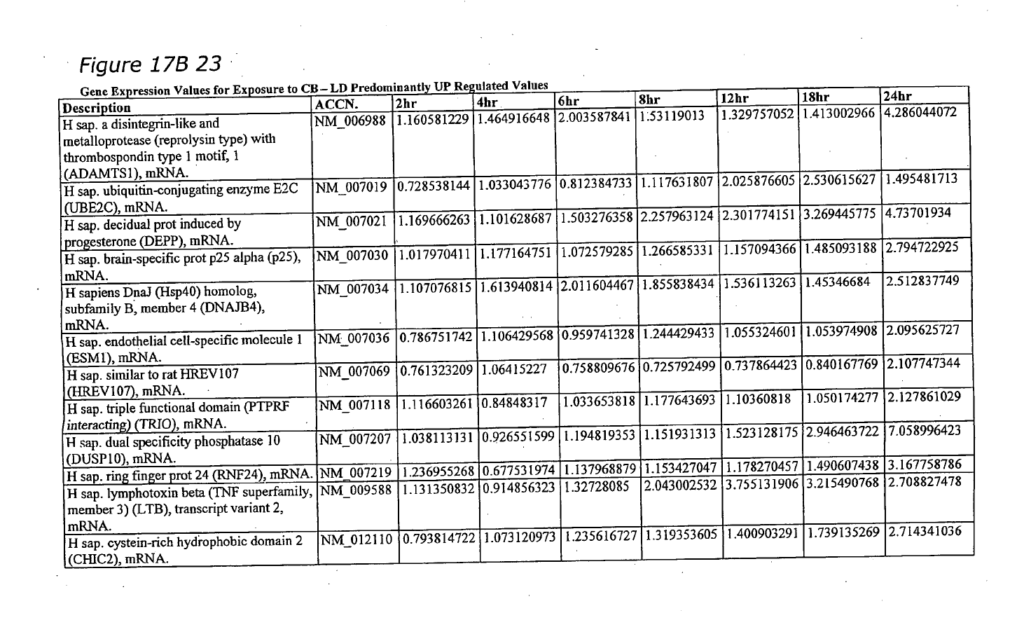

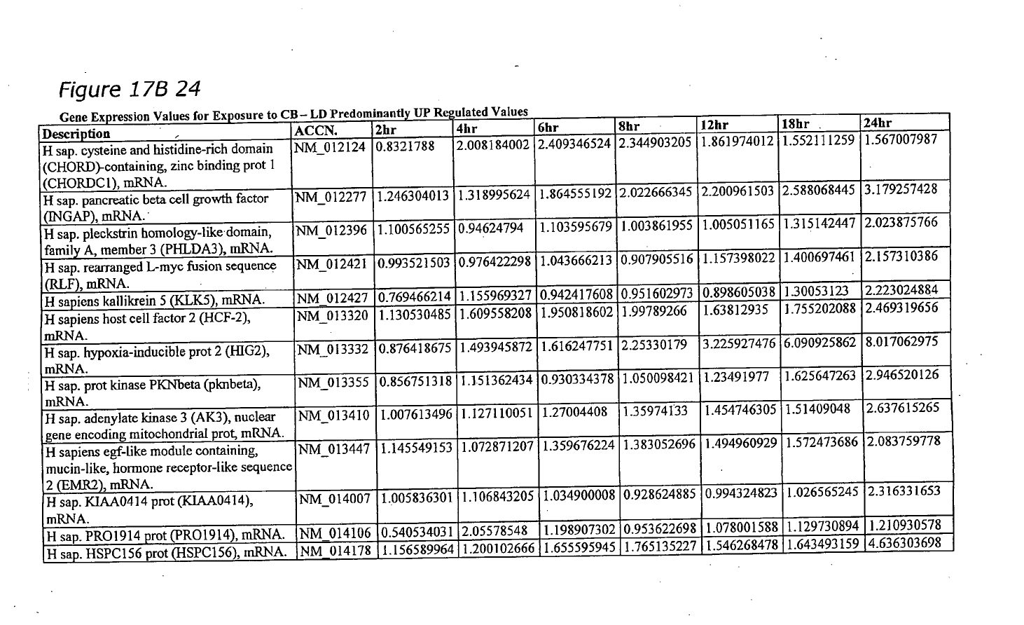

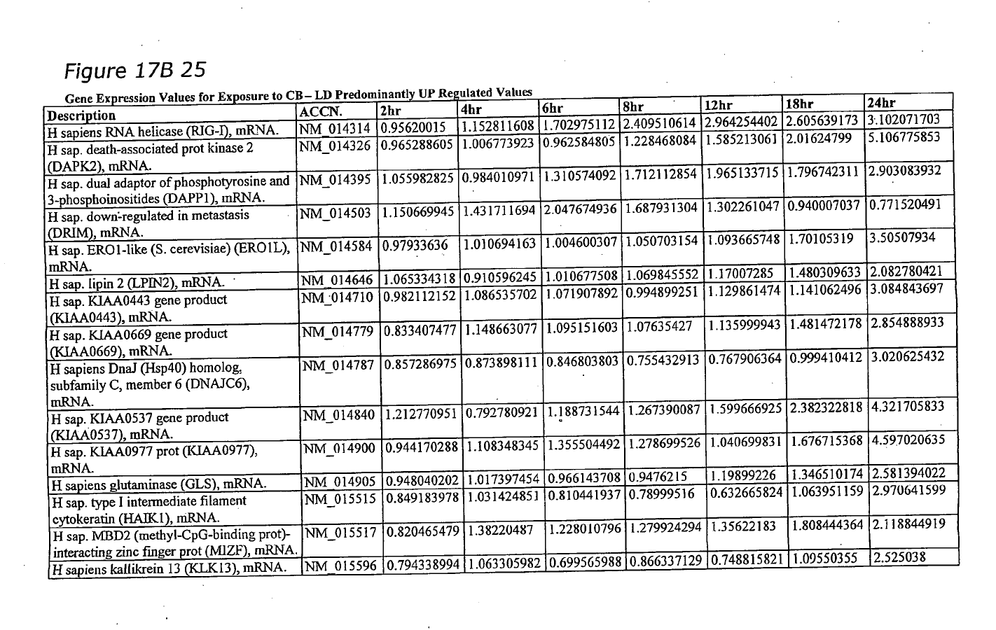

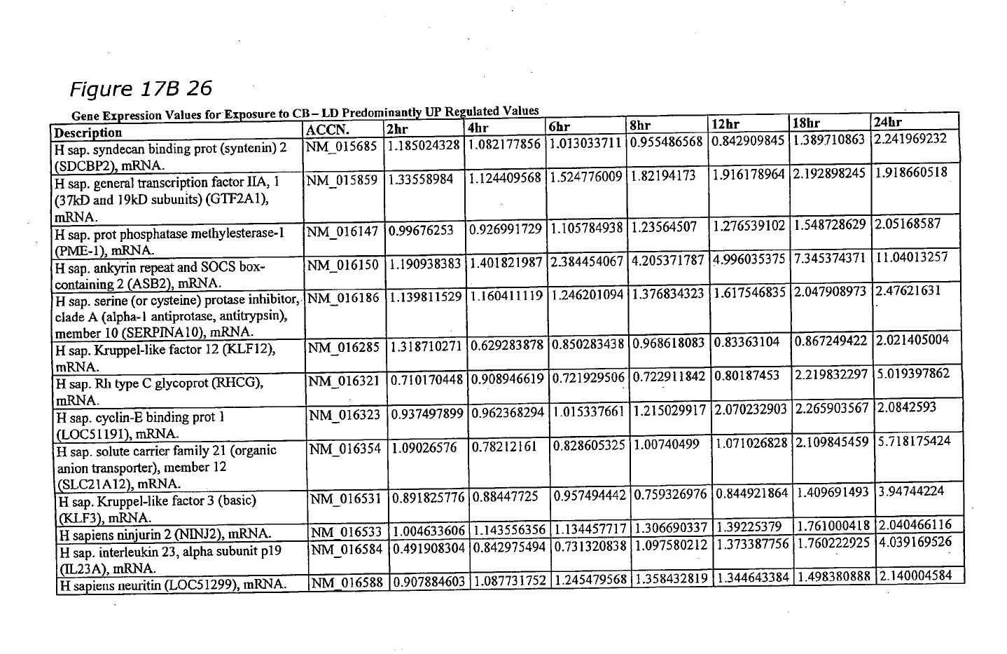

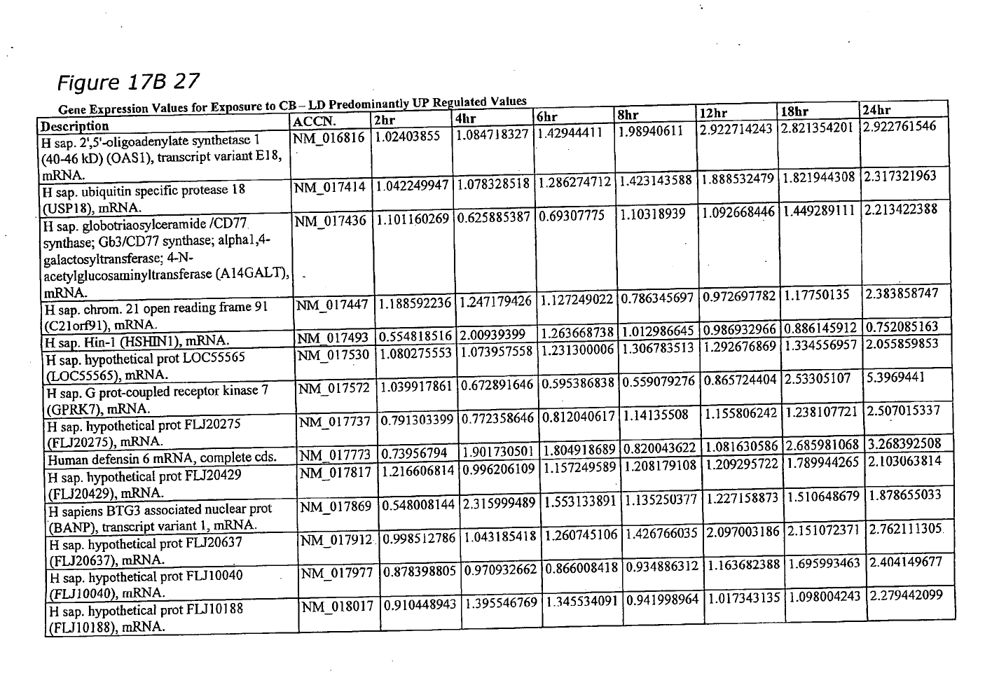

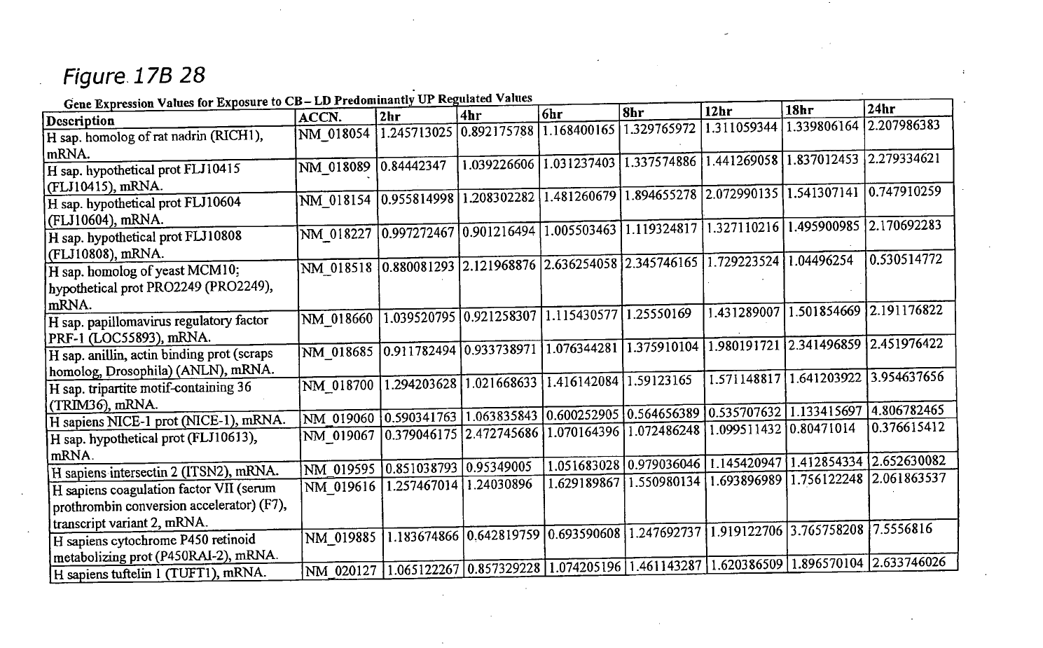

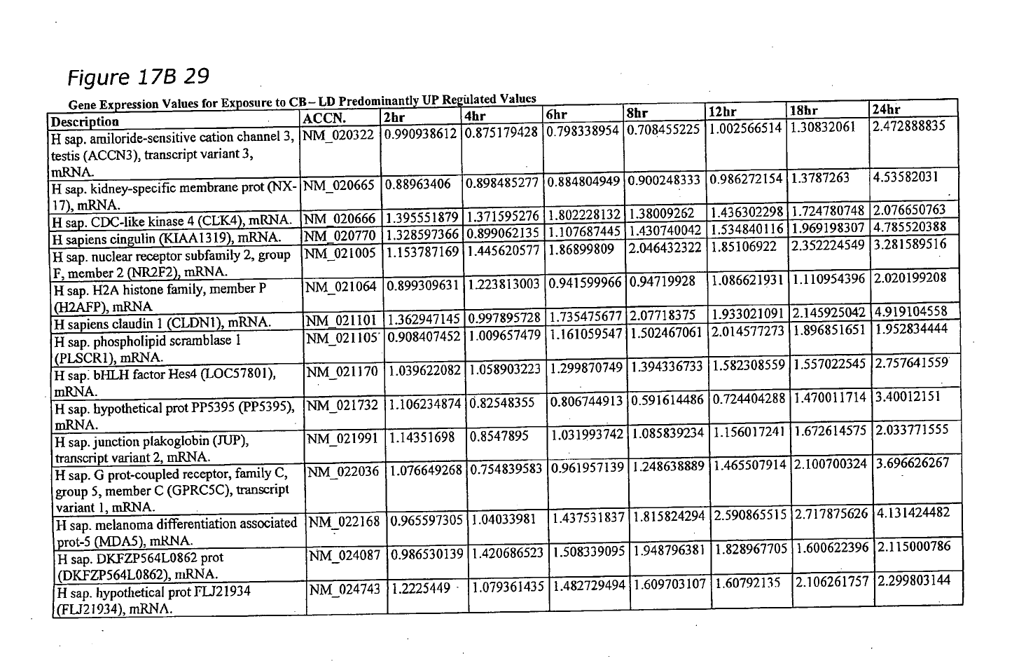

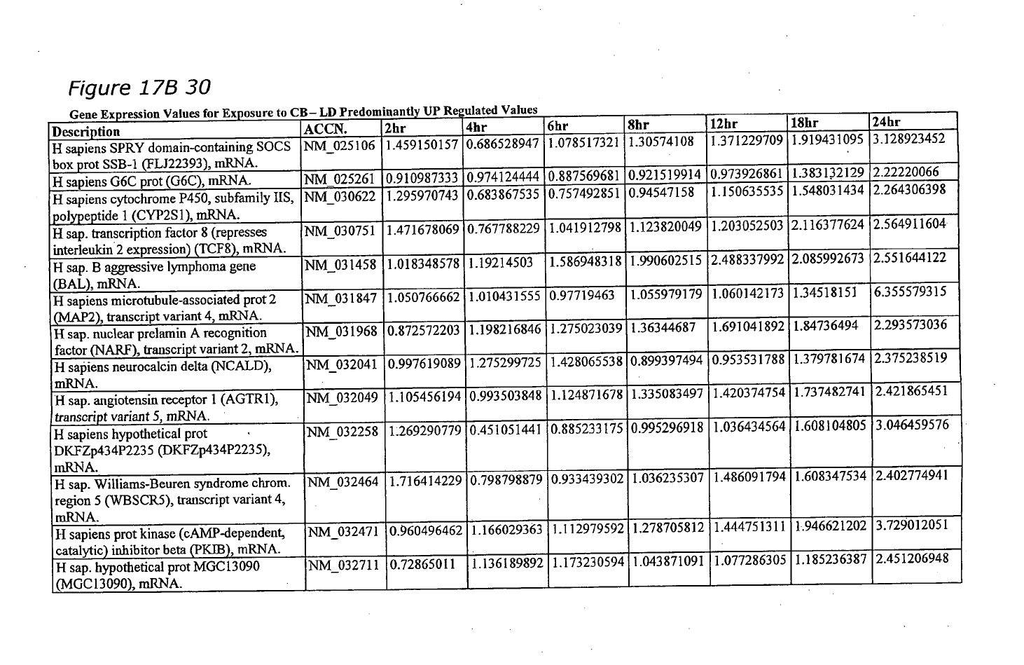

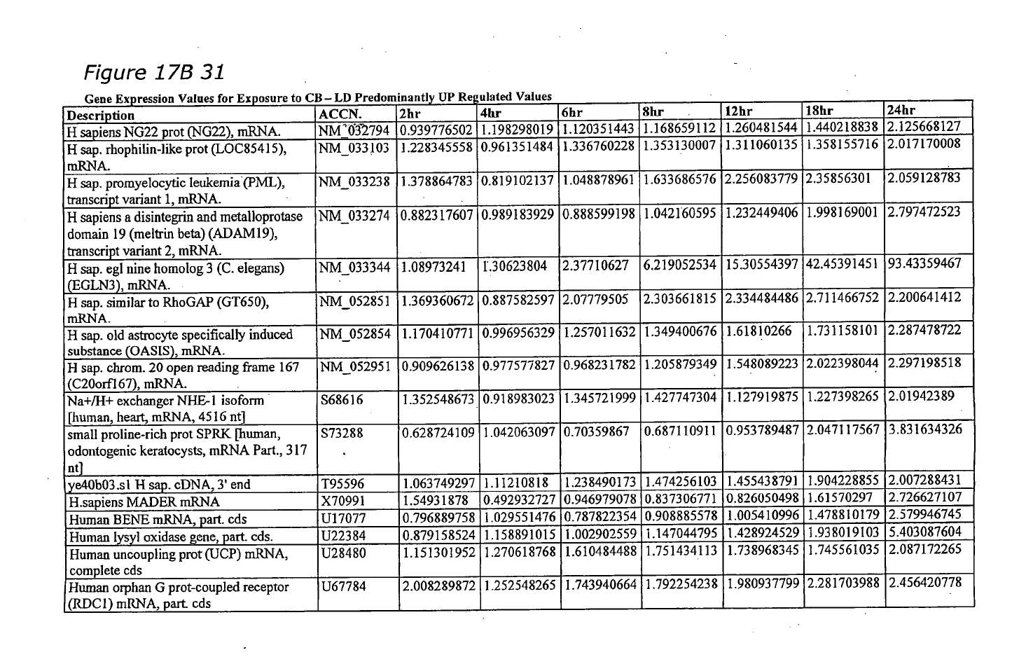

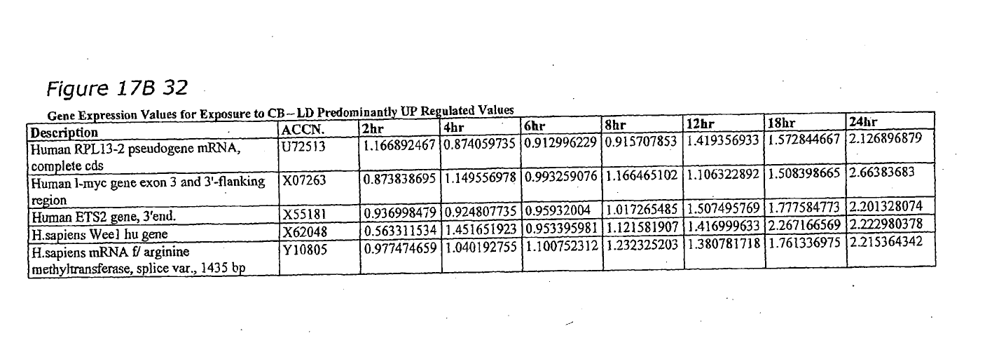

FIG. 17A1-23 presents GEM results for genes predominantly down-regulated in response to low dose CB nanoparticle exposure over time in HEK cells.

FIG. 17B1-32 presents GEM results for genes predominantly up-regulated in response to low dose CB nanoparticle exposure over time in HEK cells.

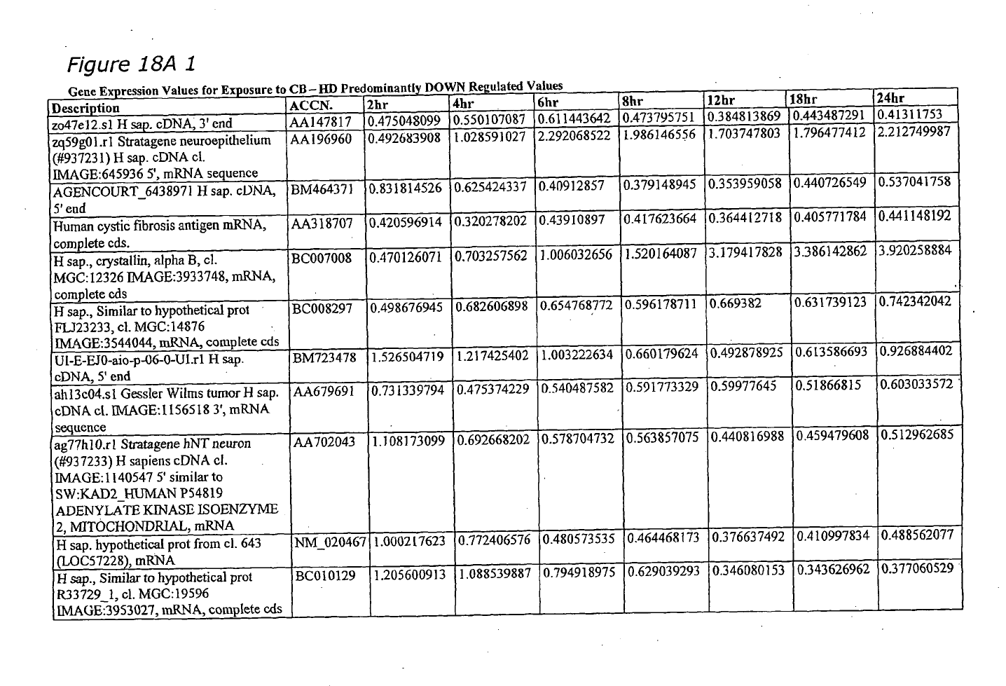

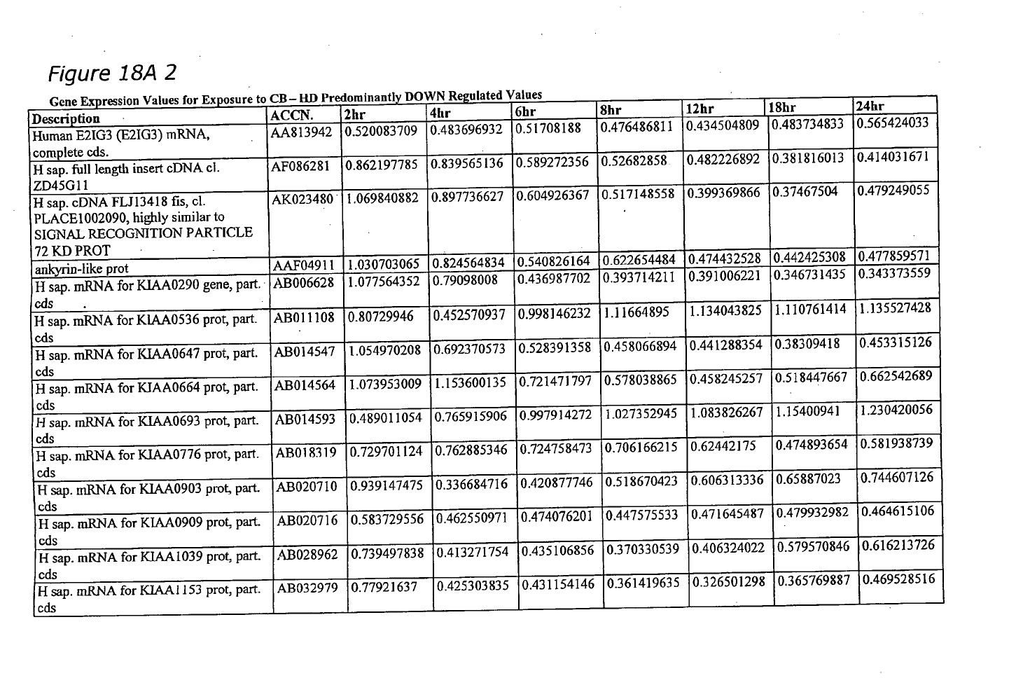

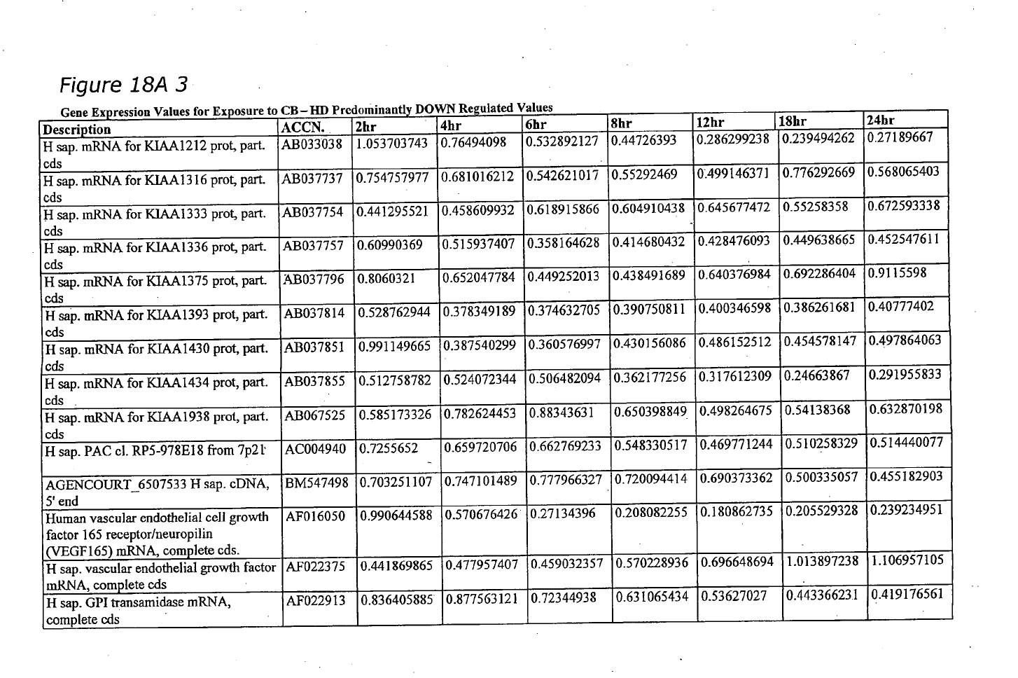

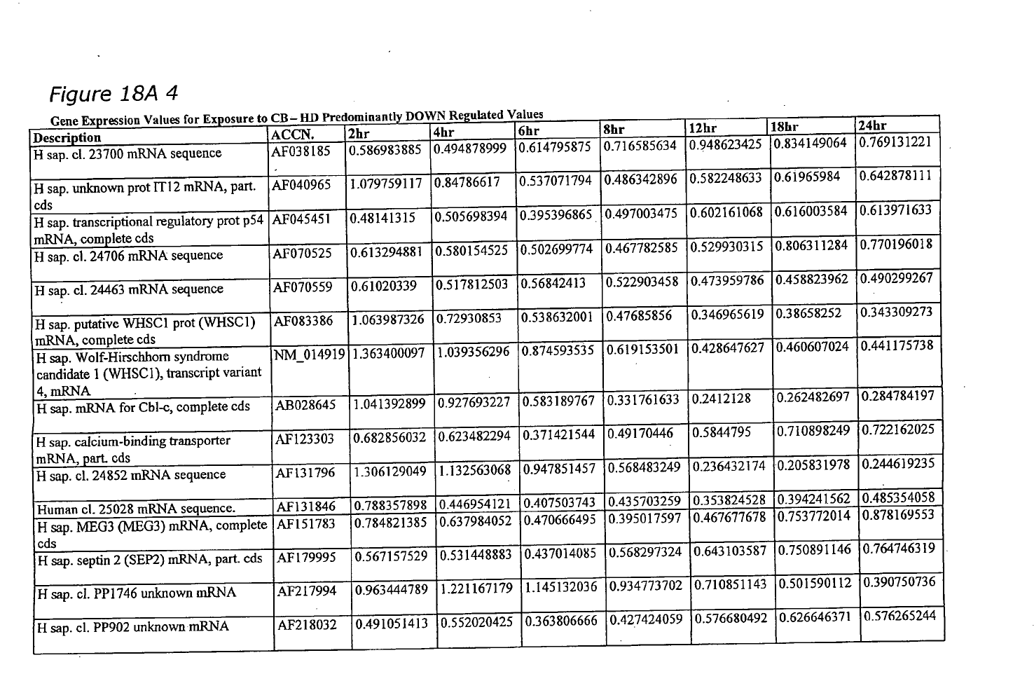

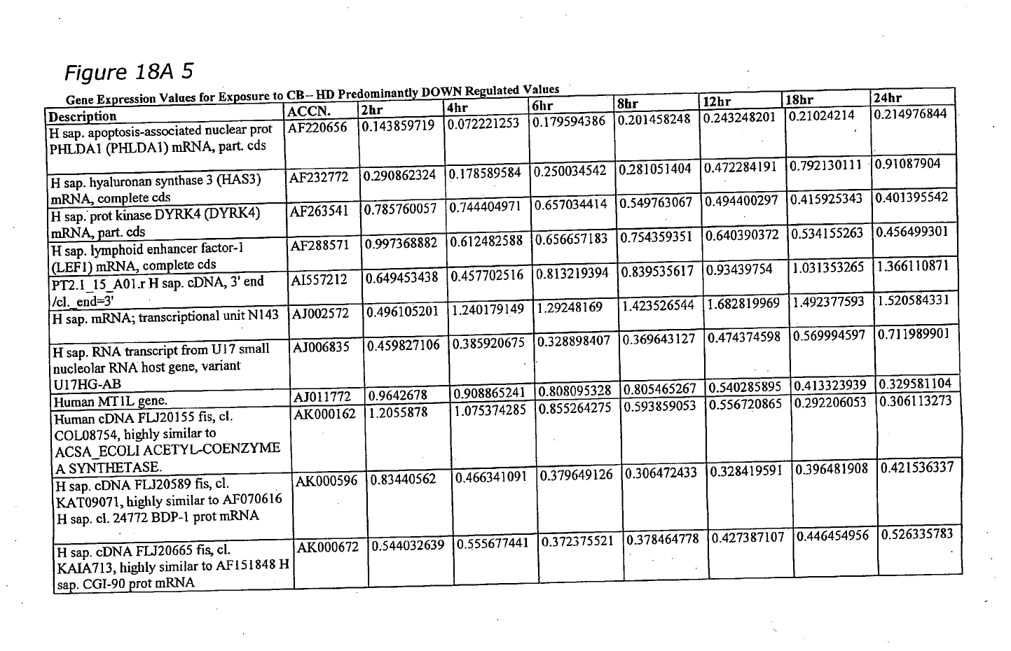

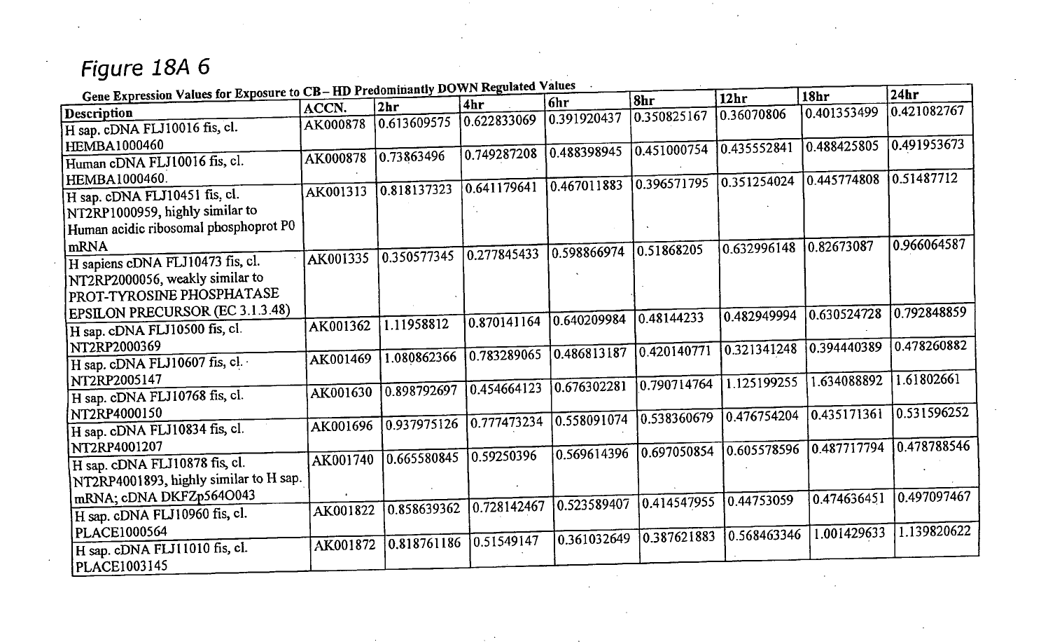

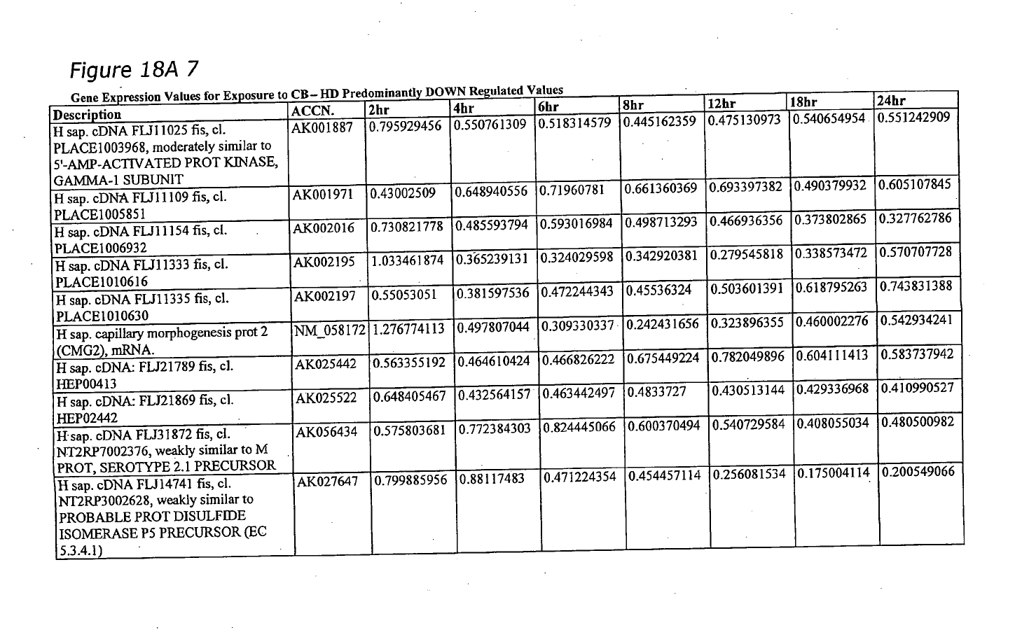

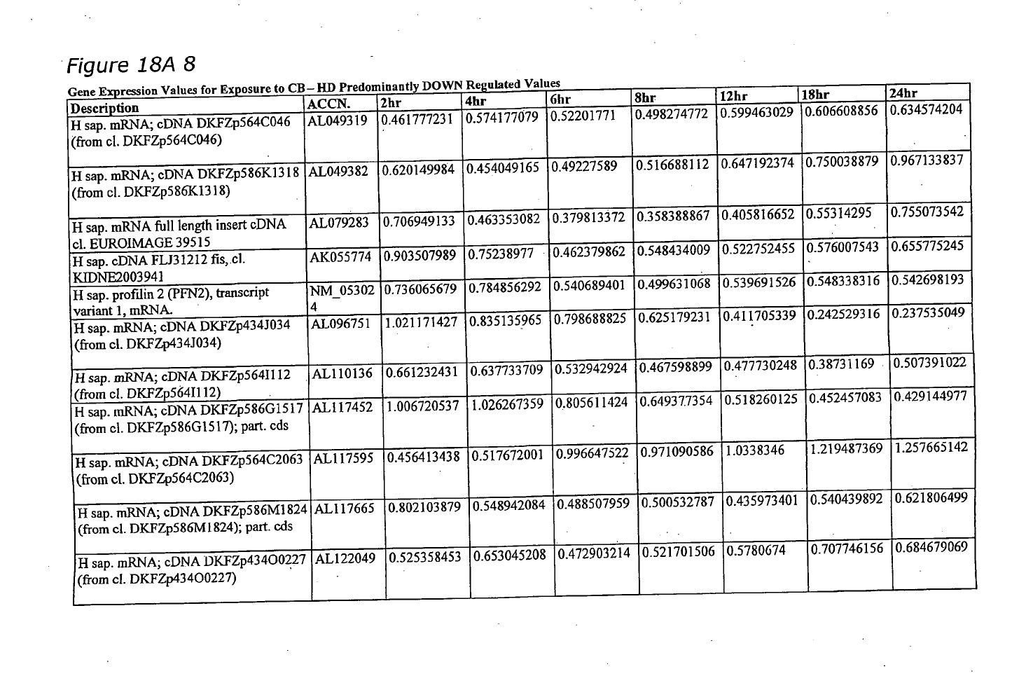

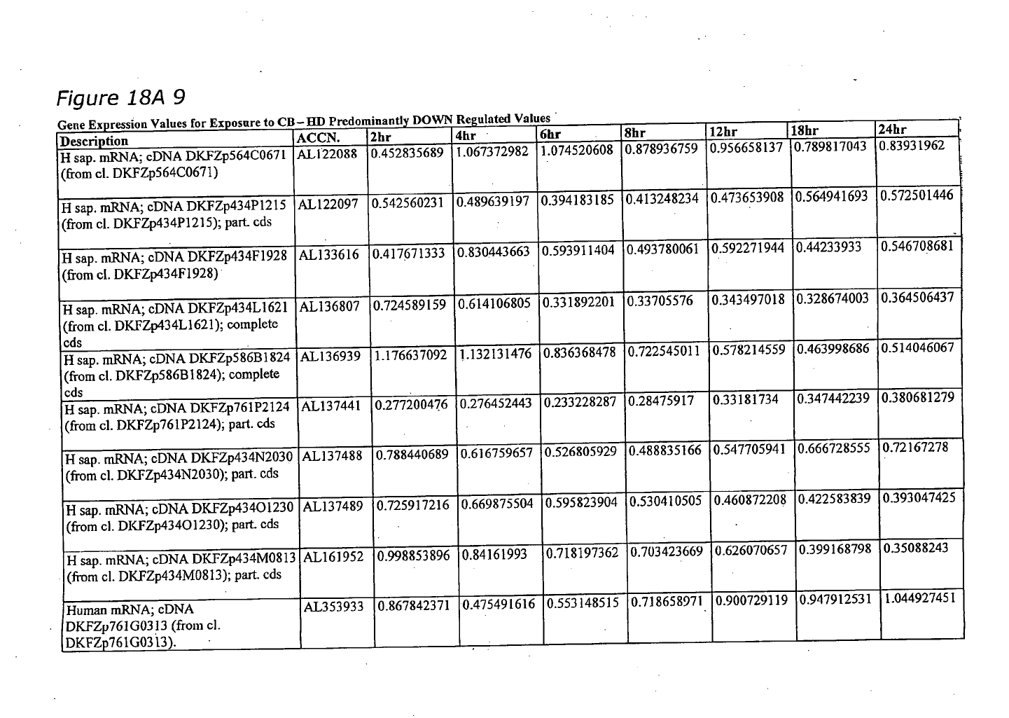

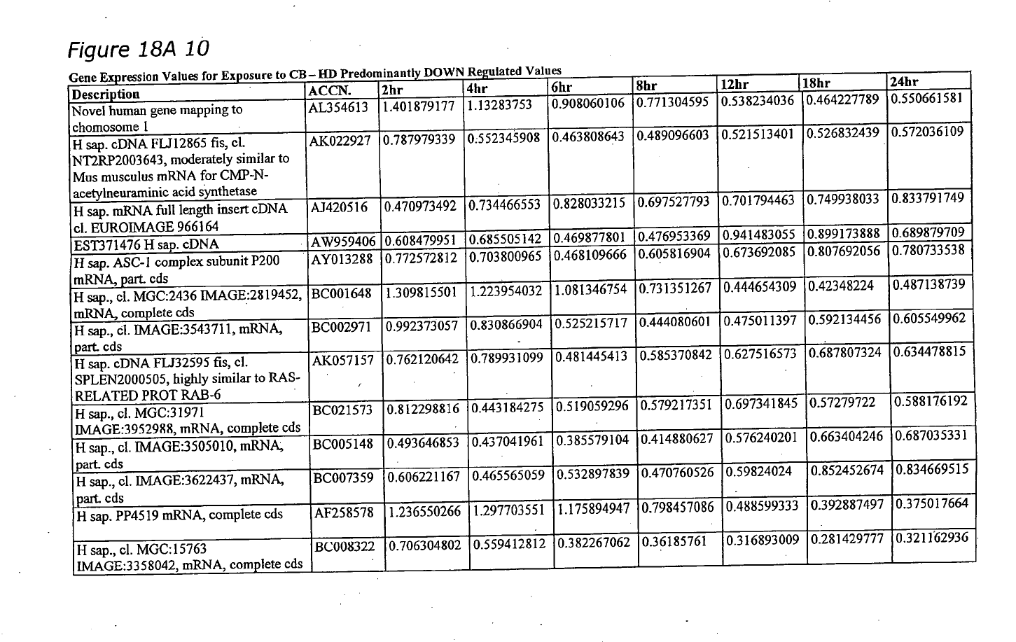

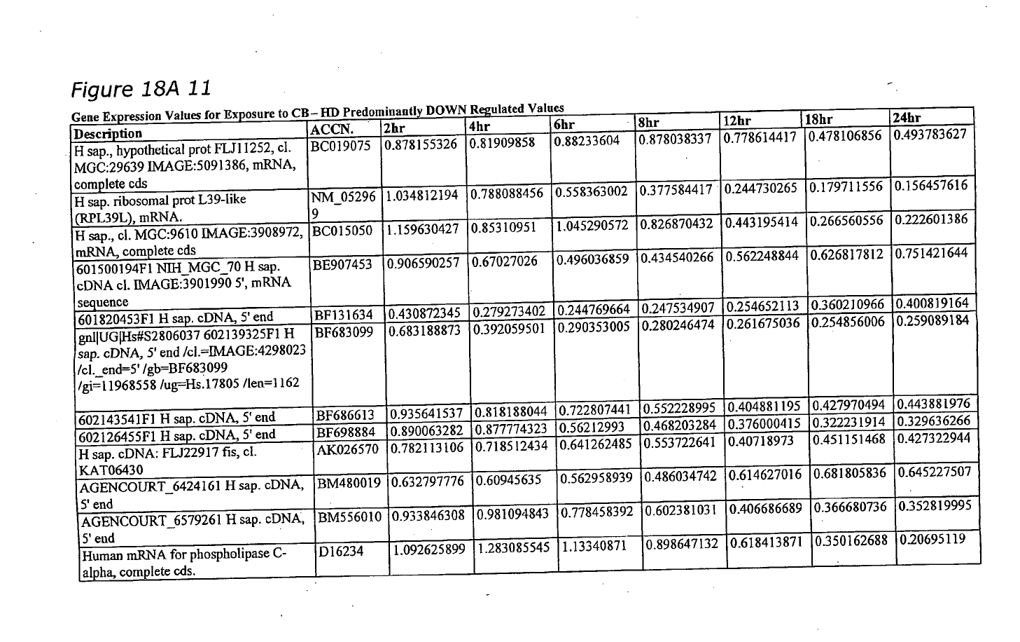

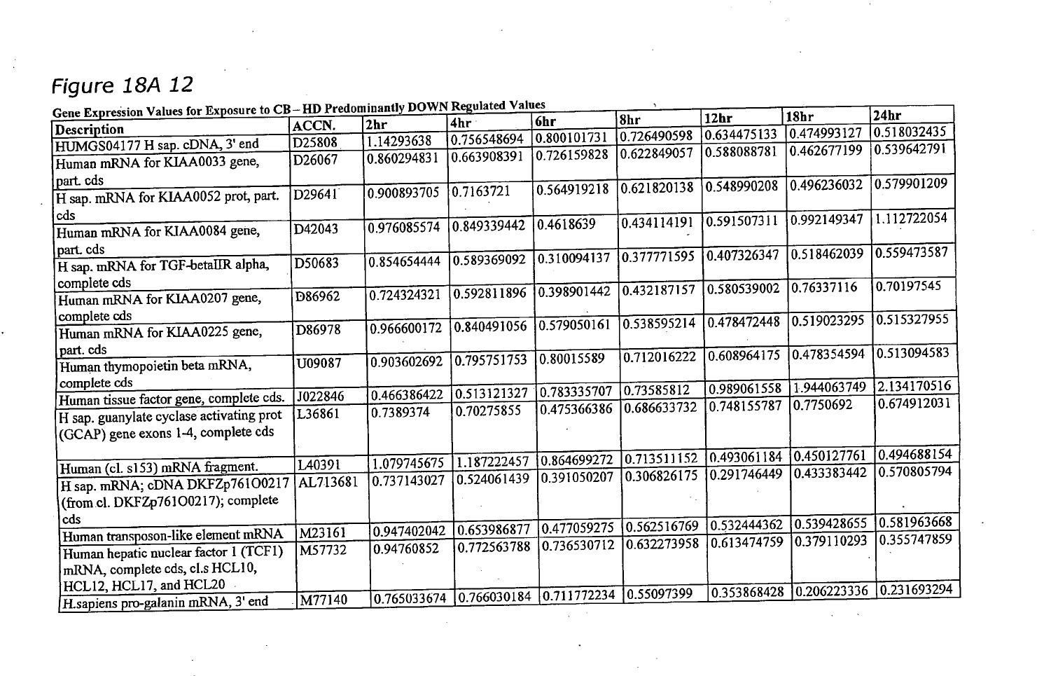

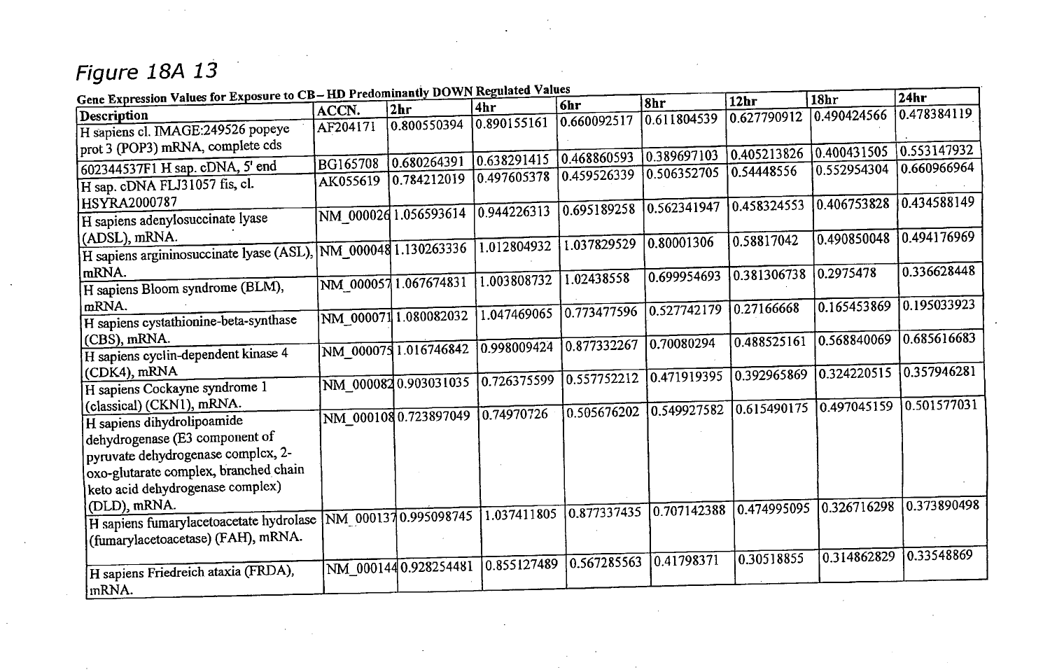

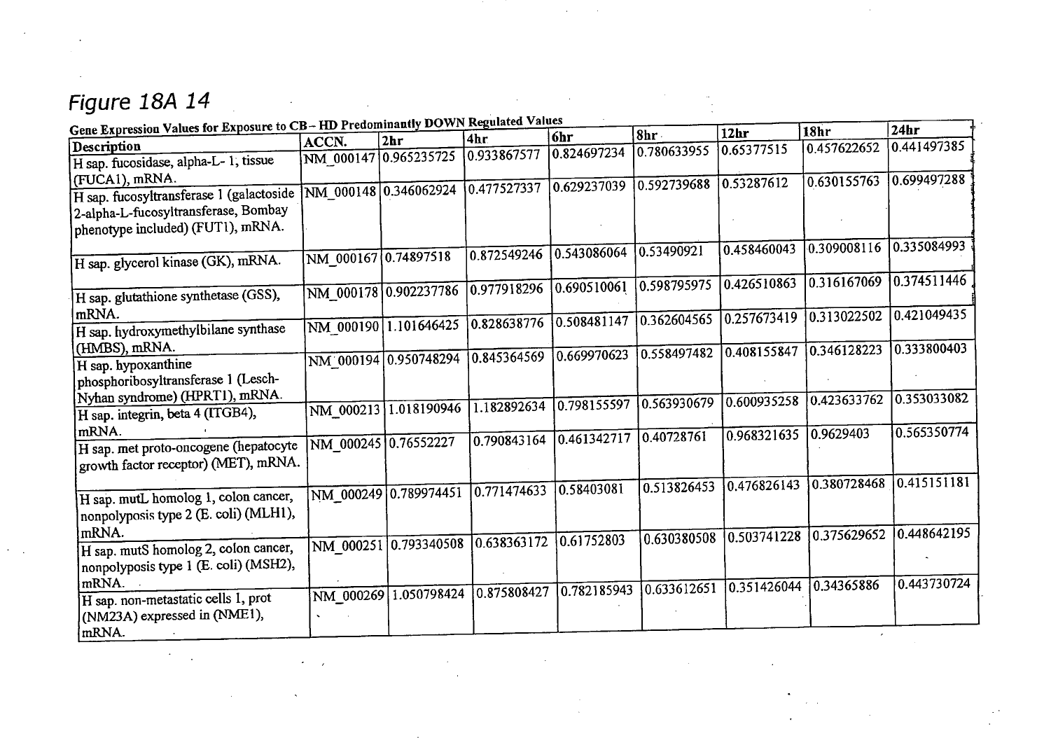

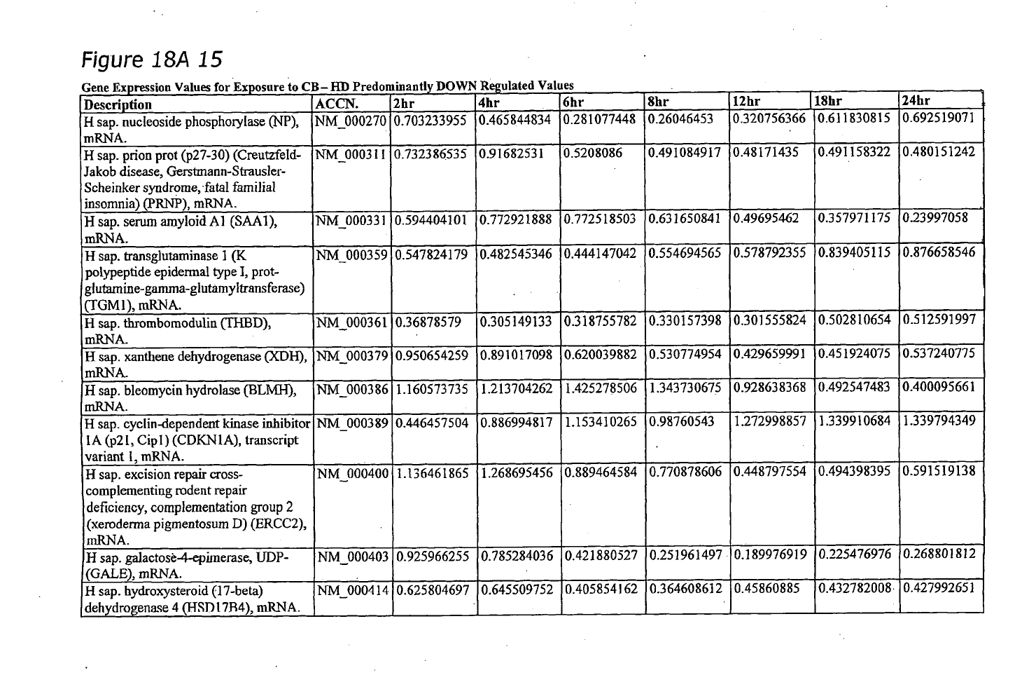

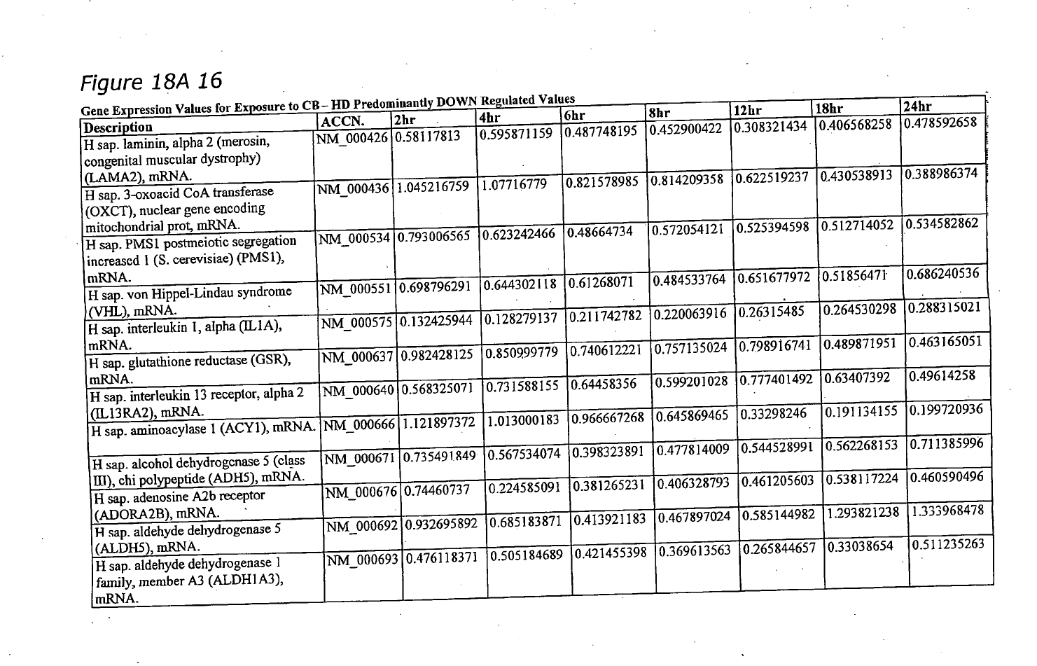

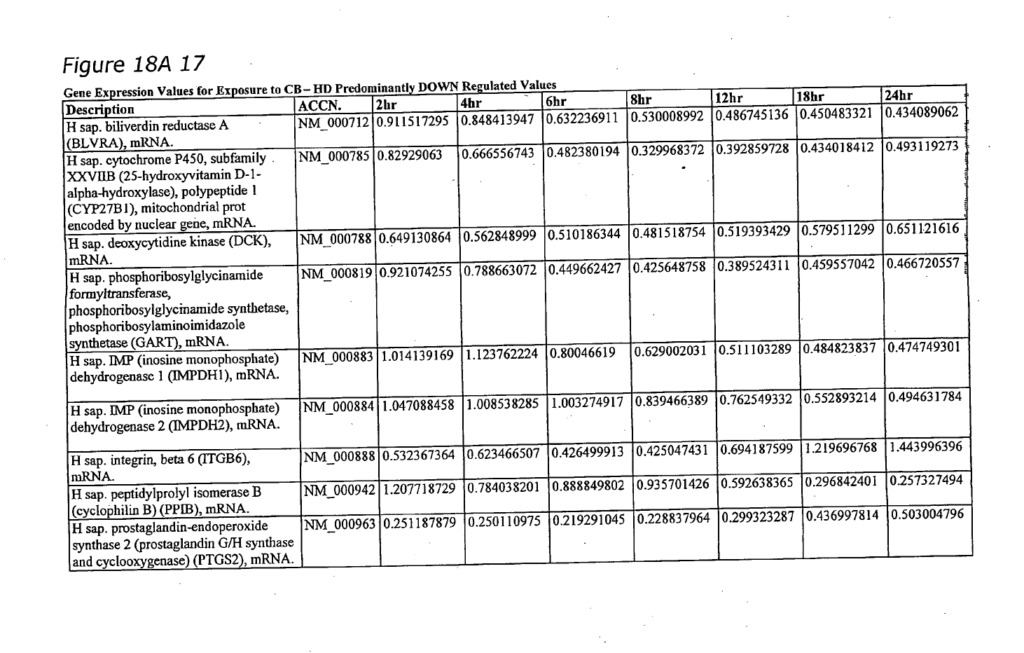

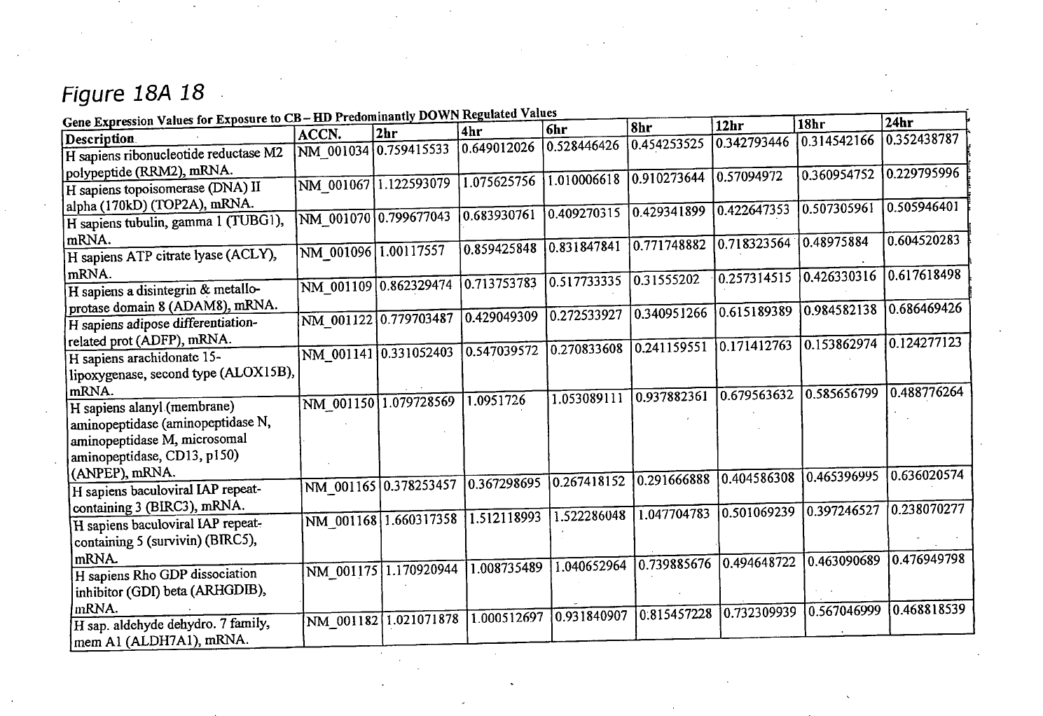

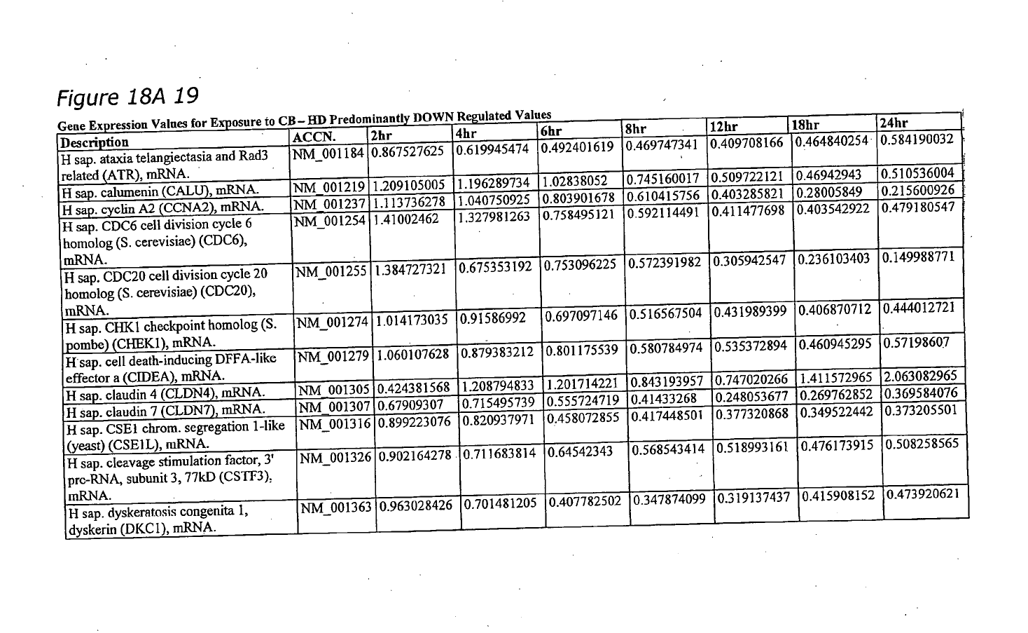

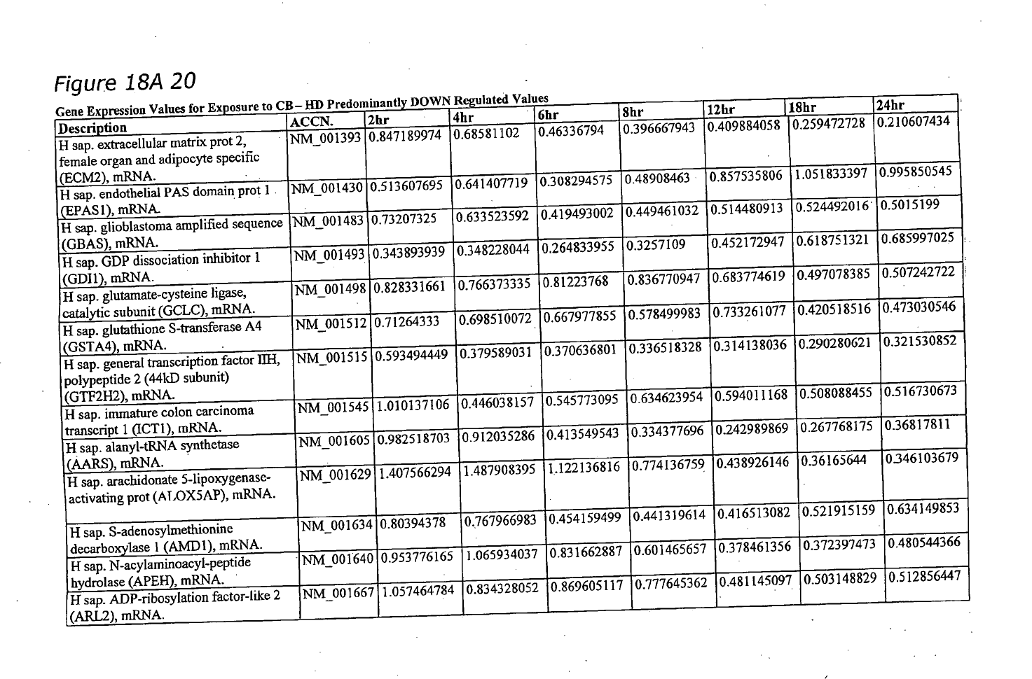

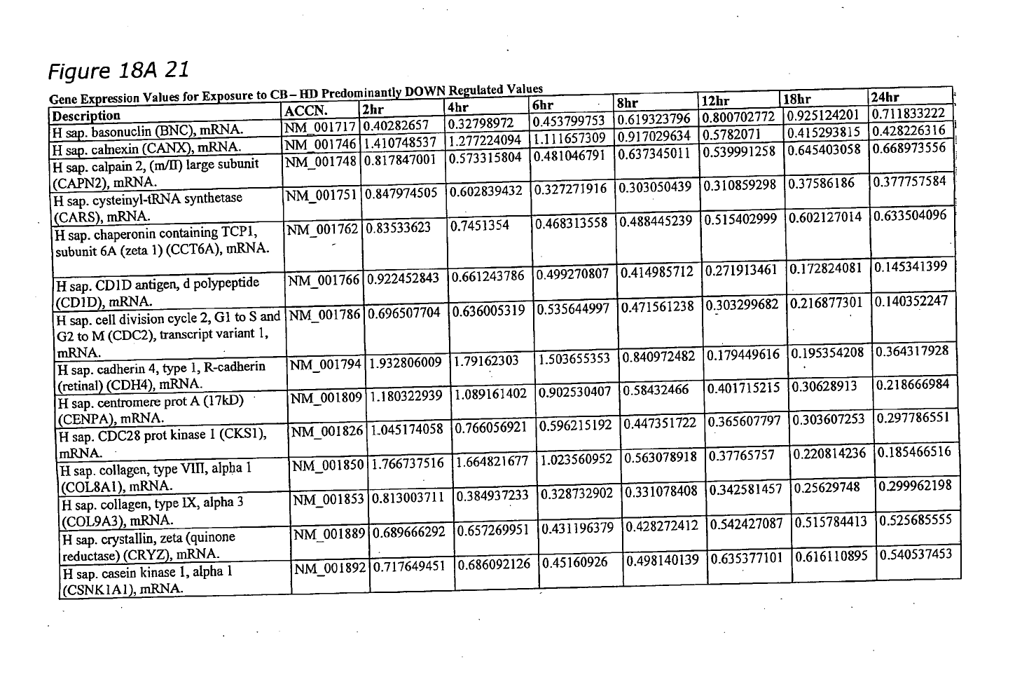

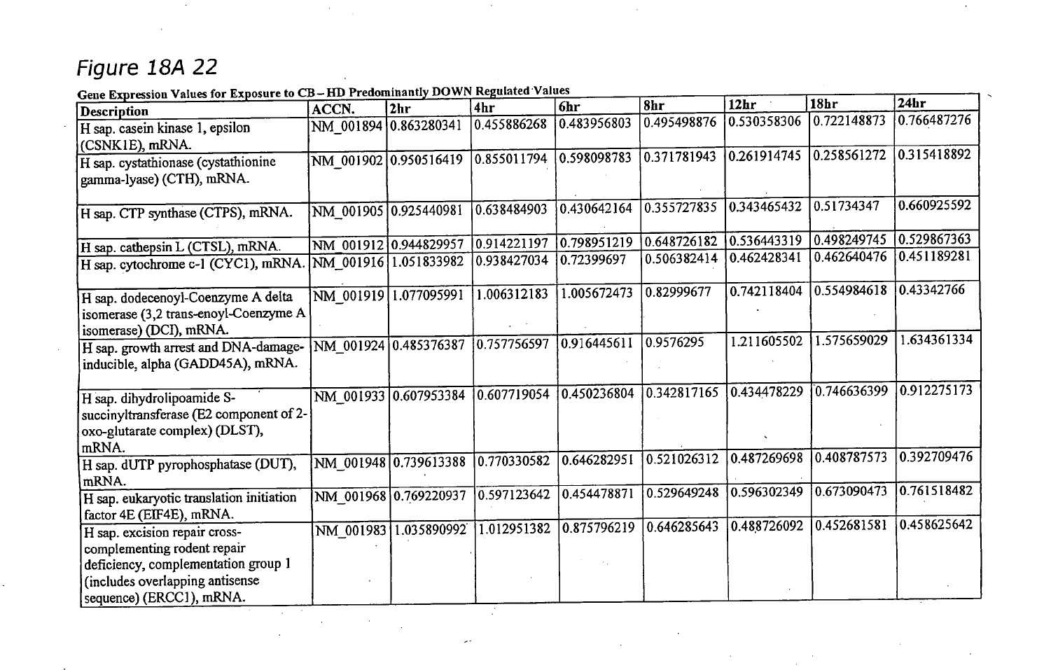

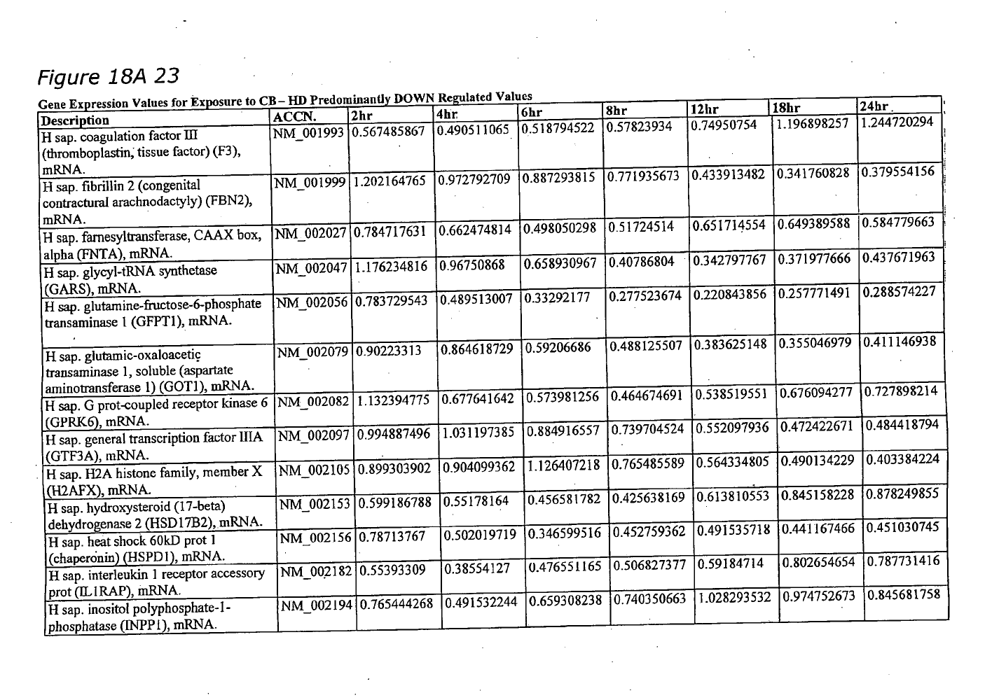

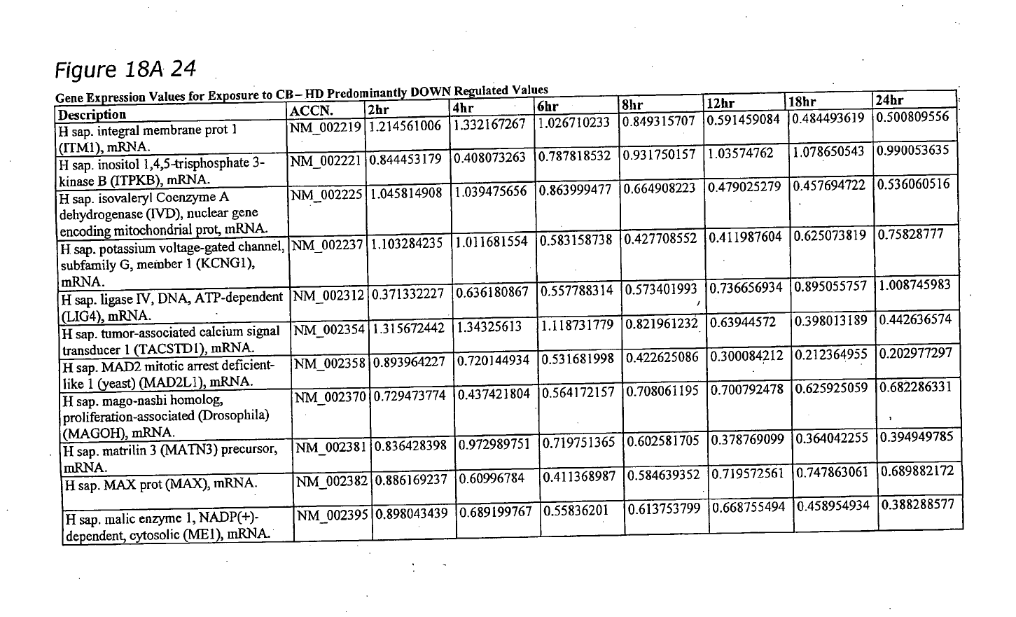

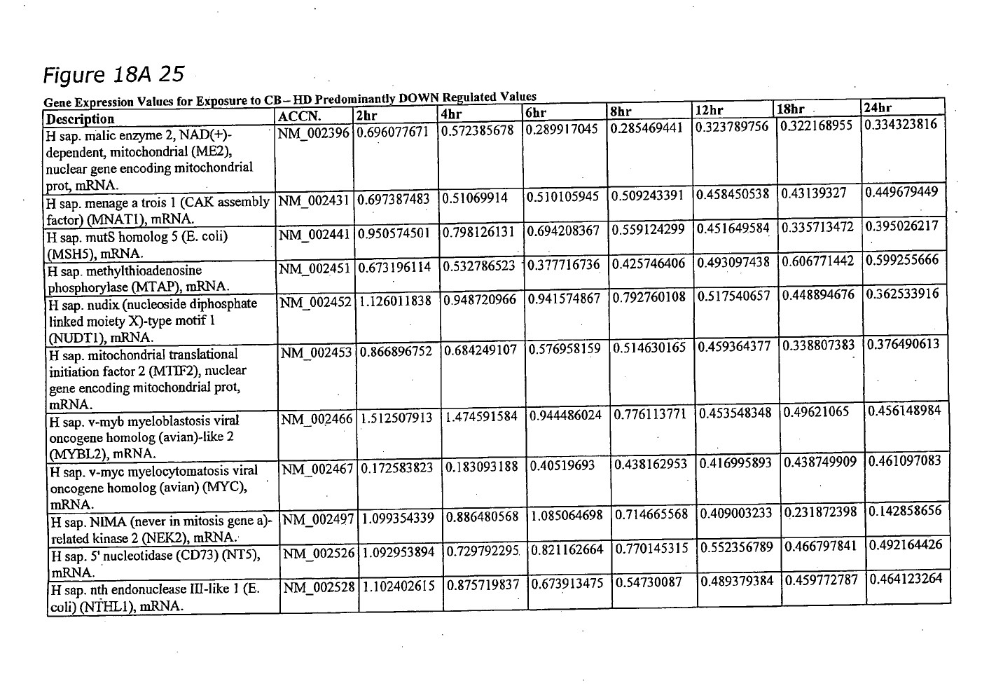

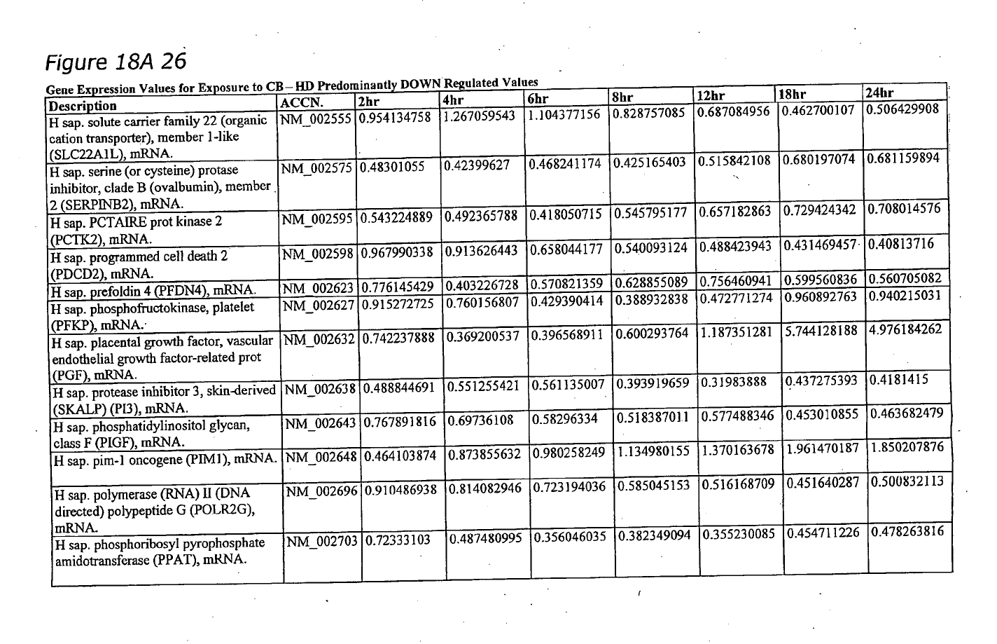

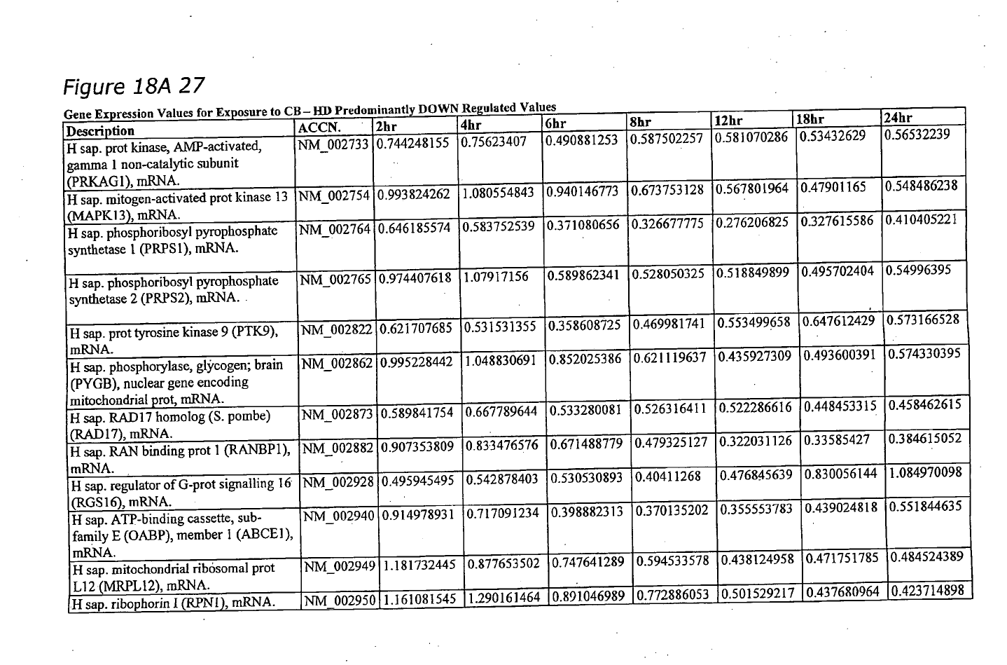

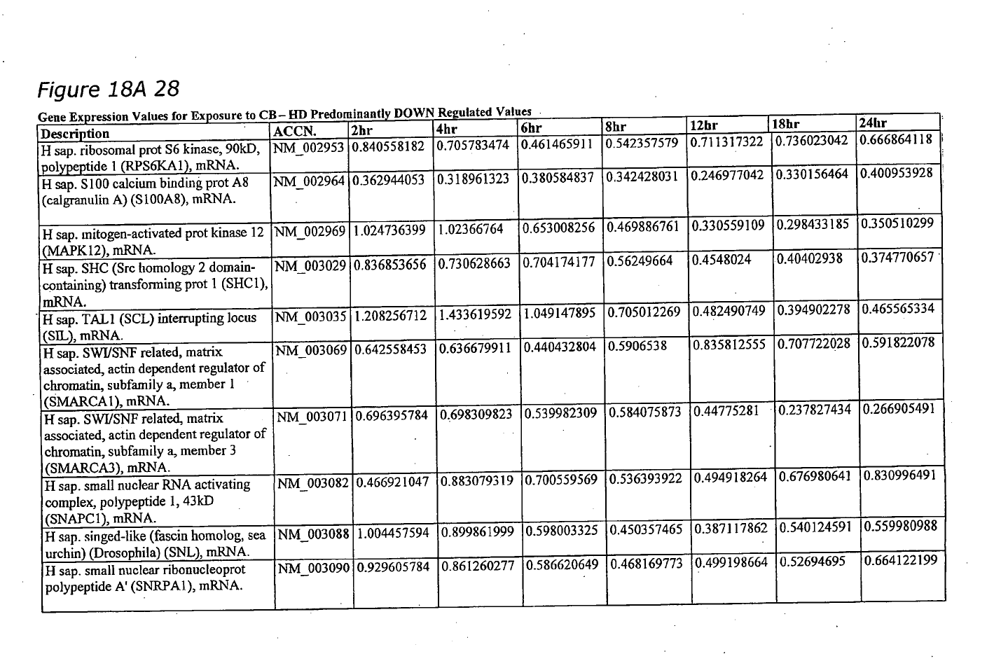

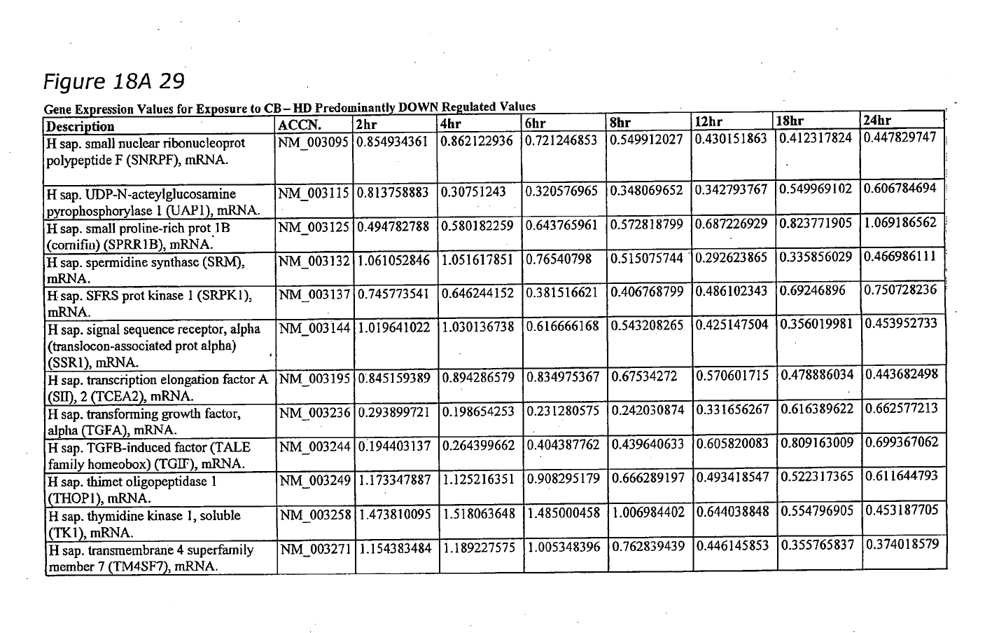

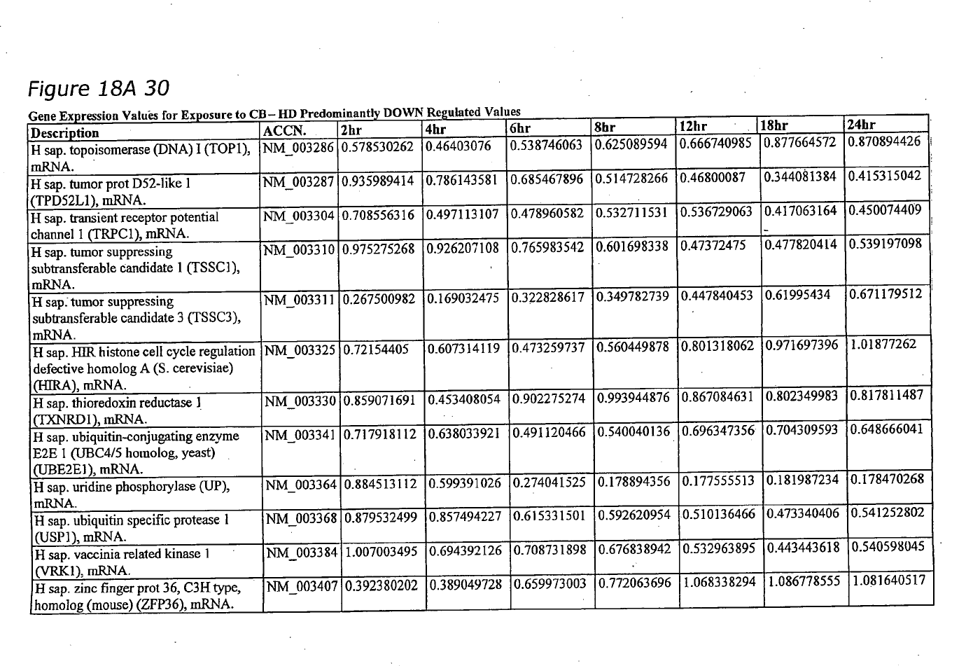

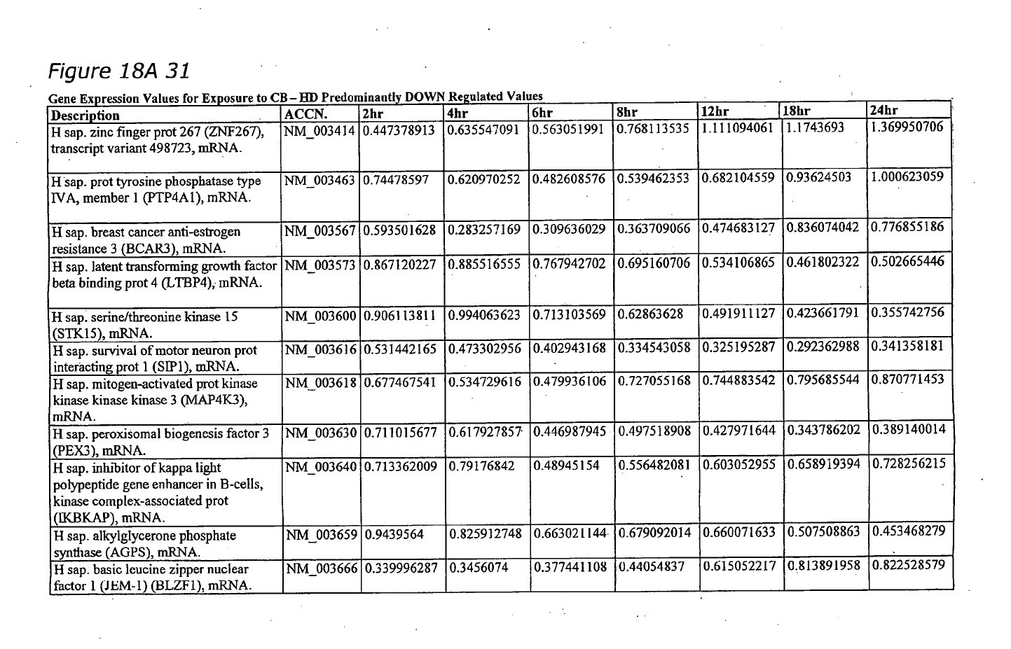

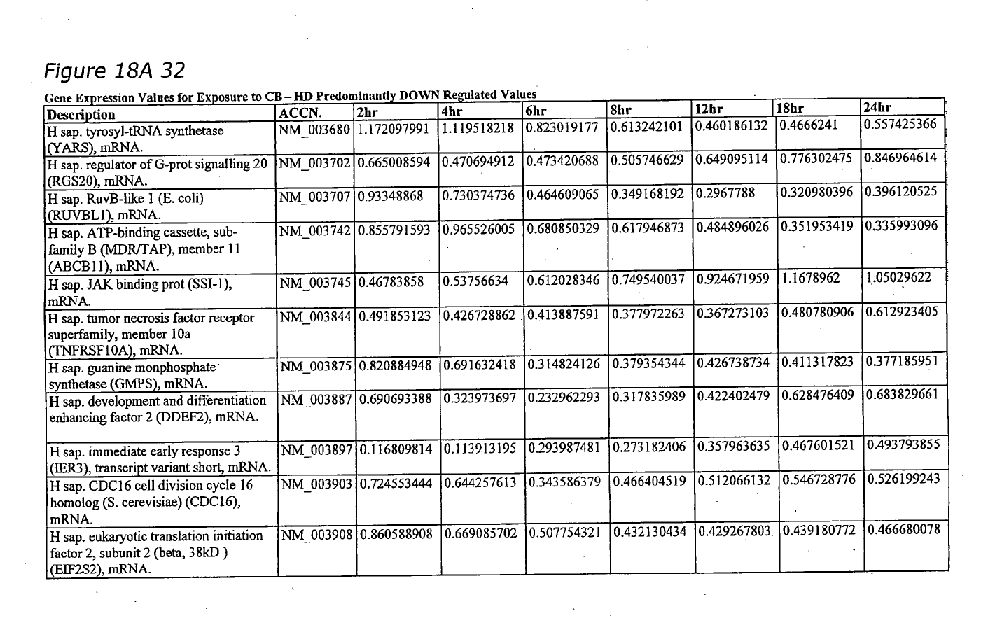

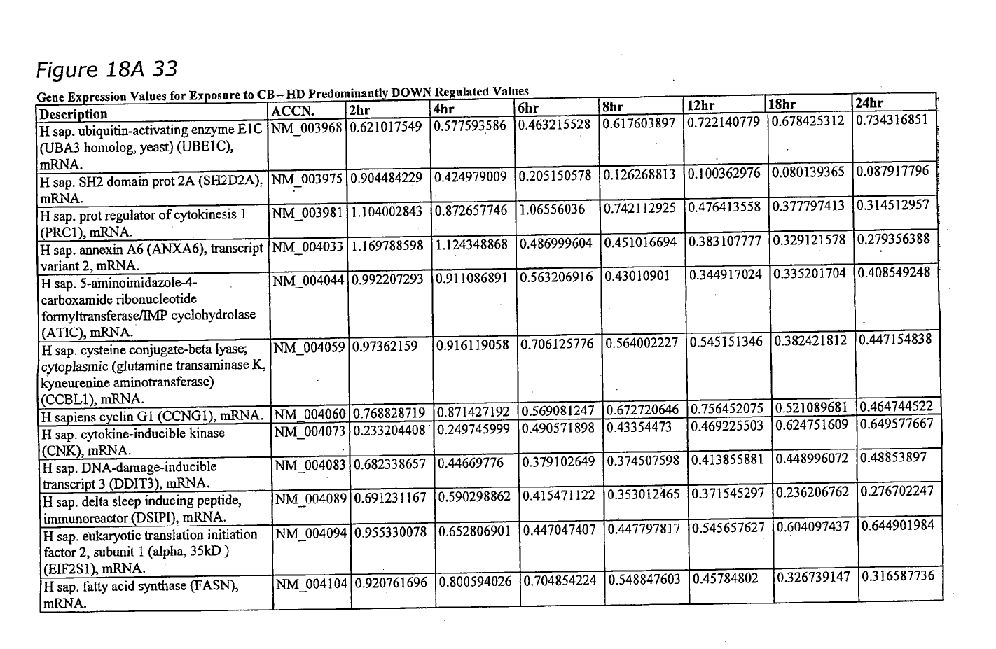

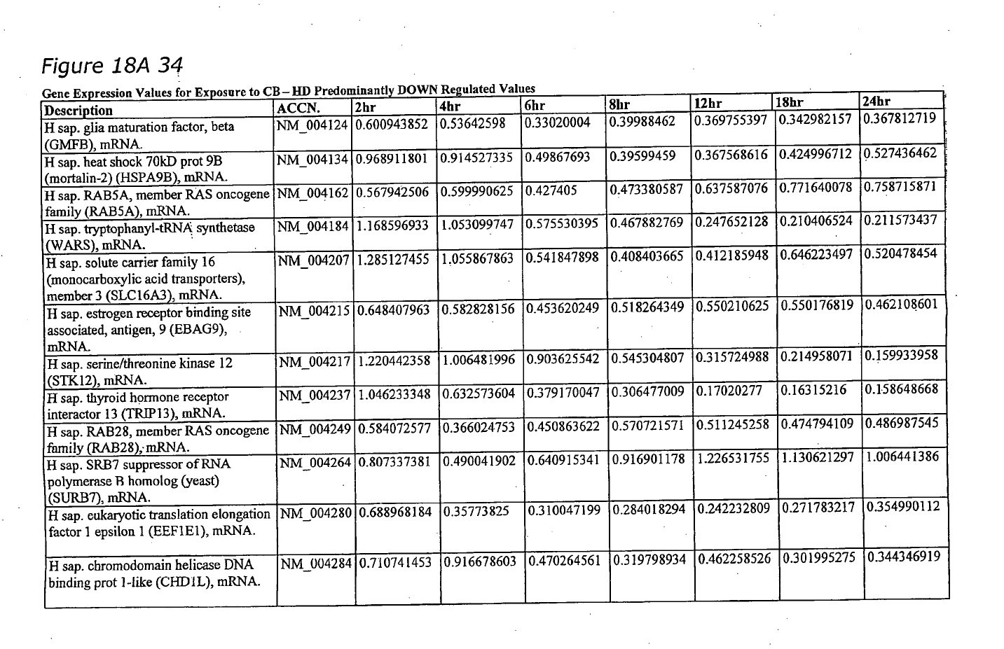

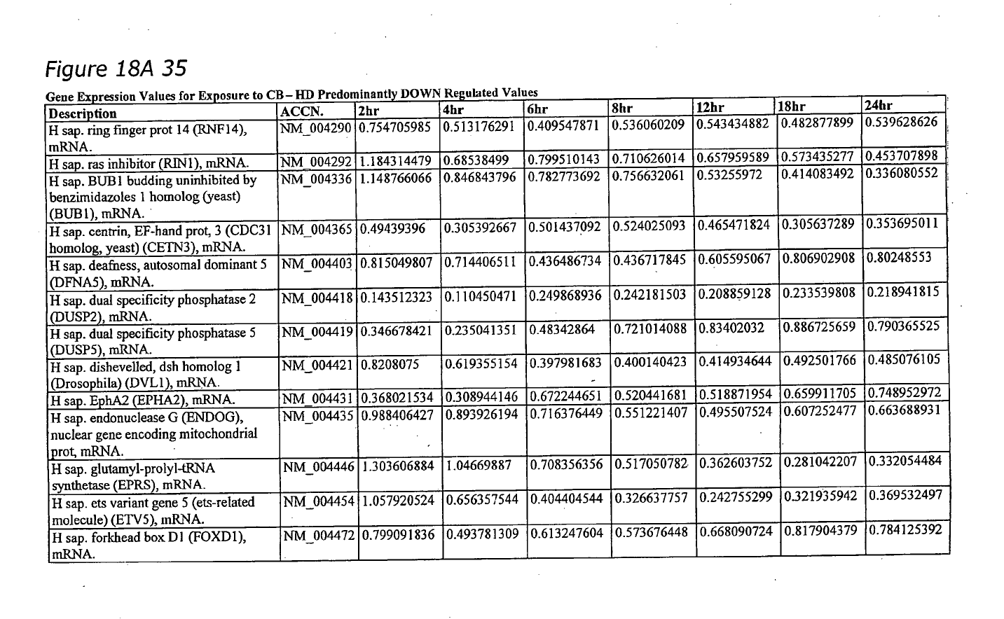

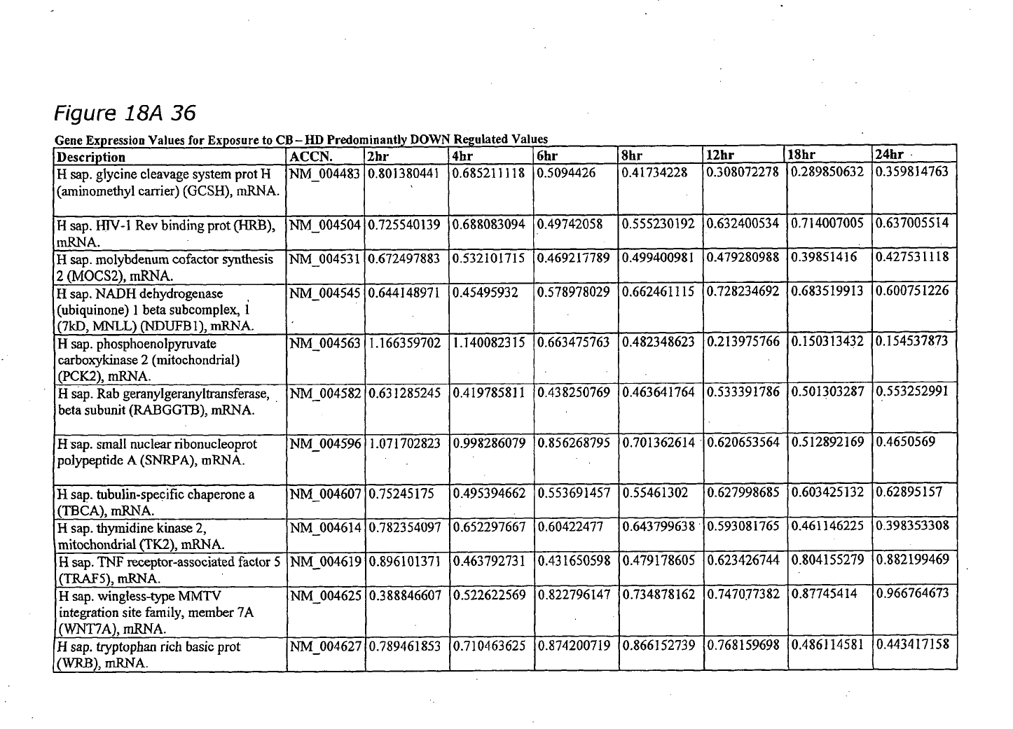

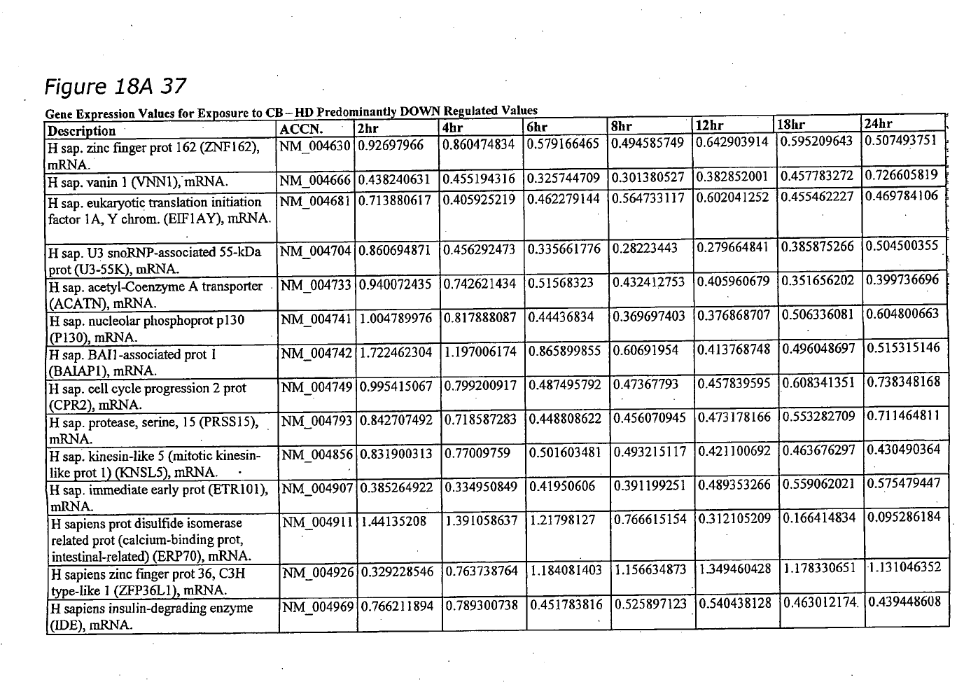

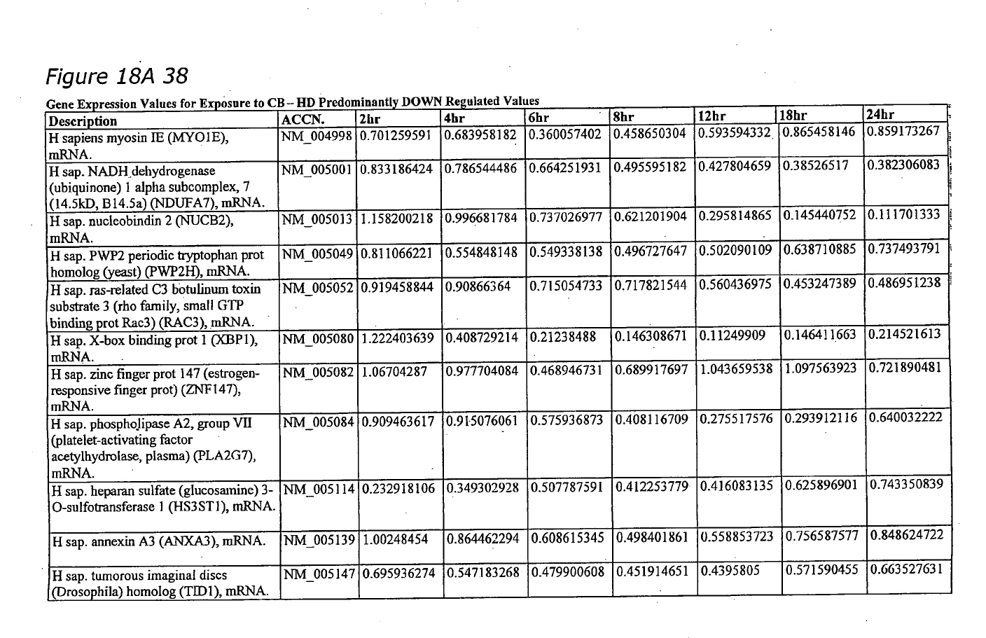

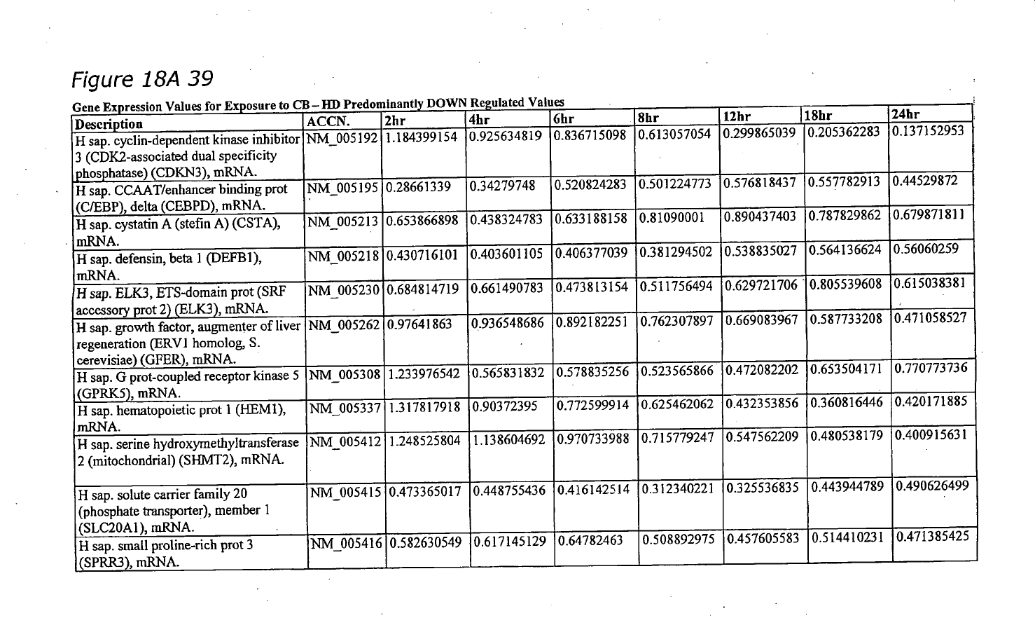

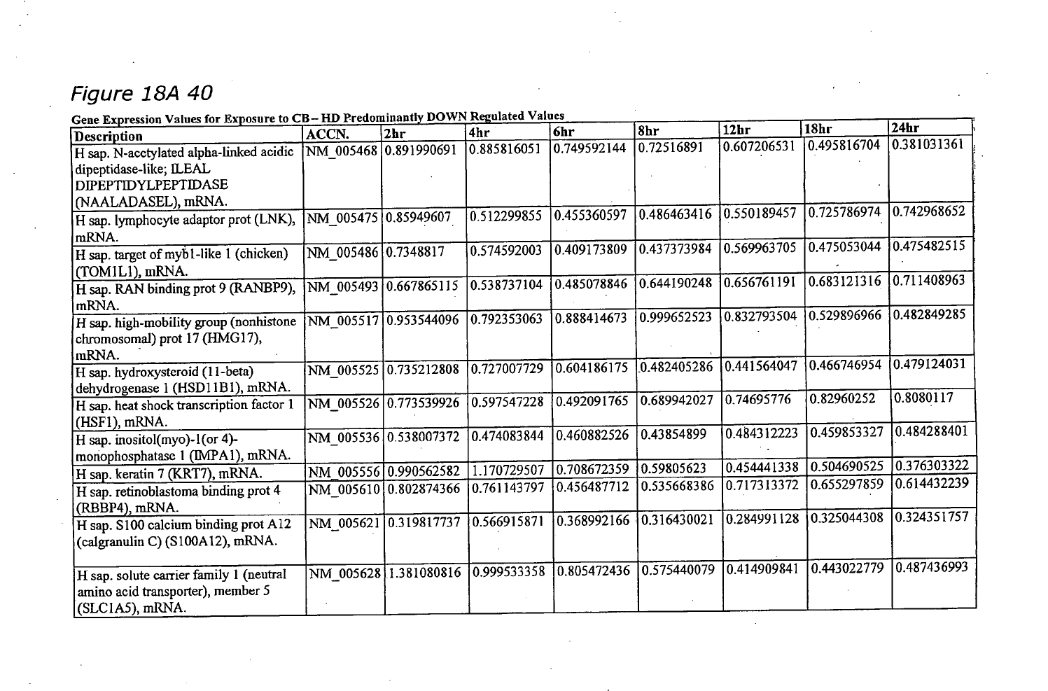

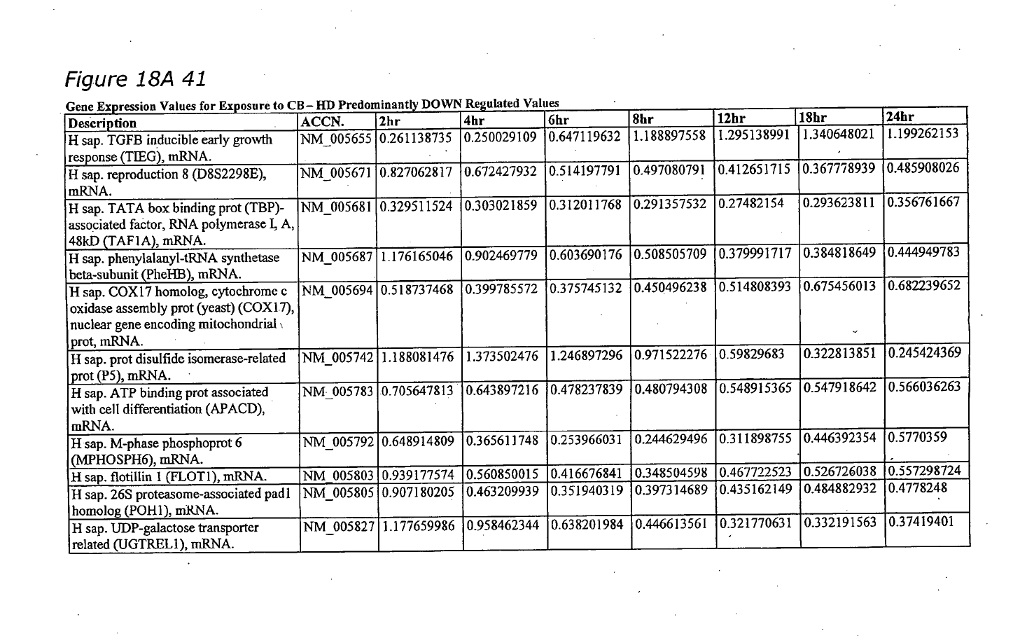

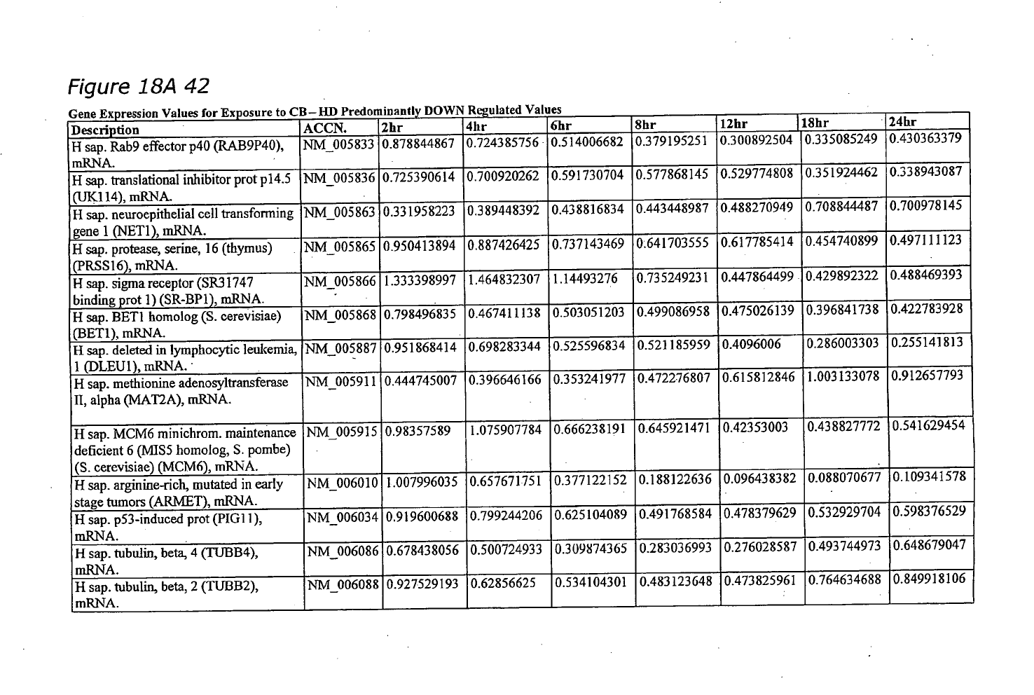

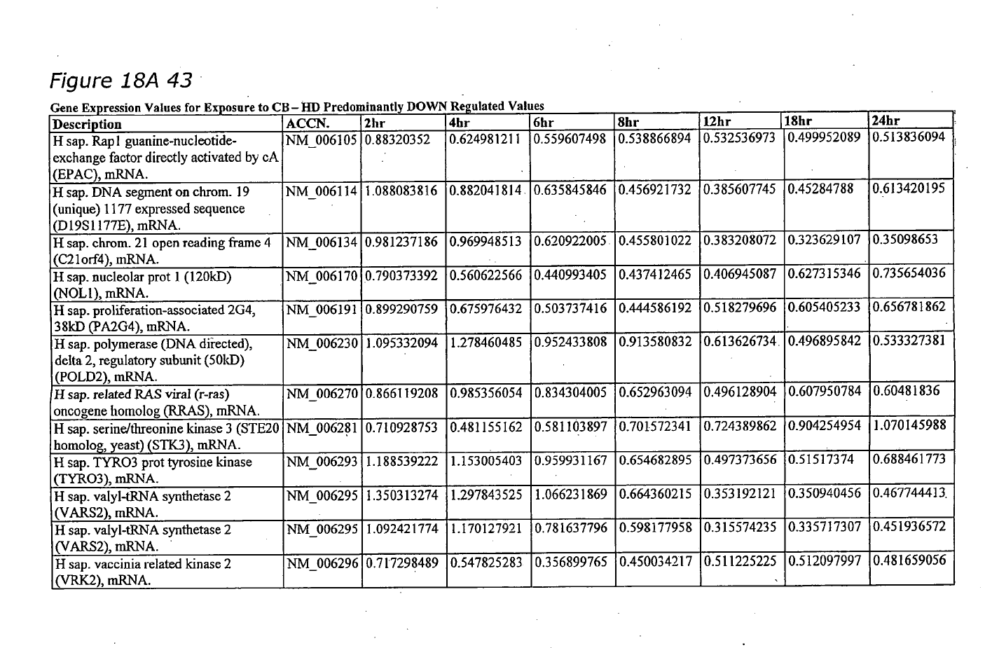

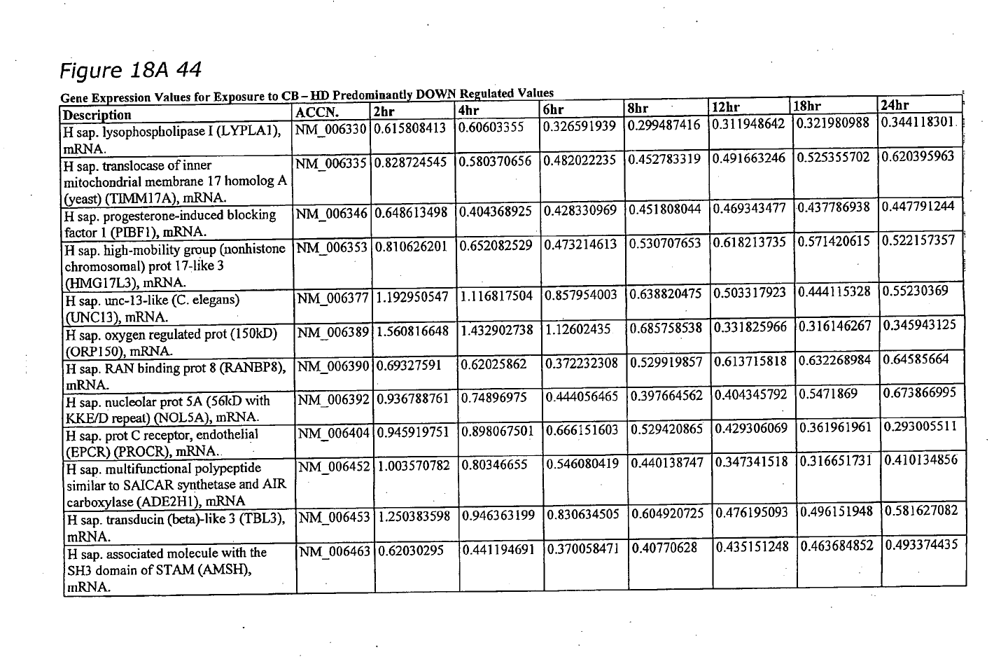

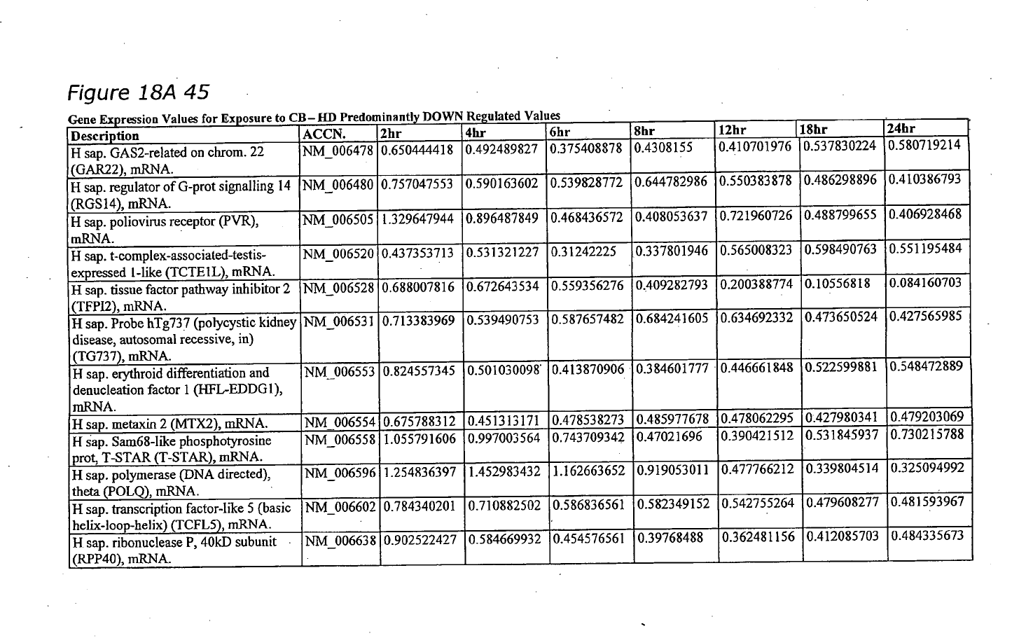

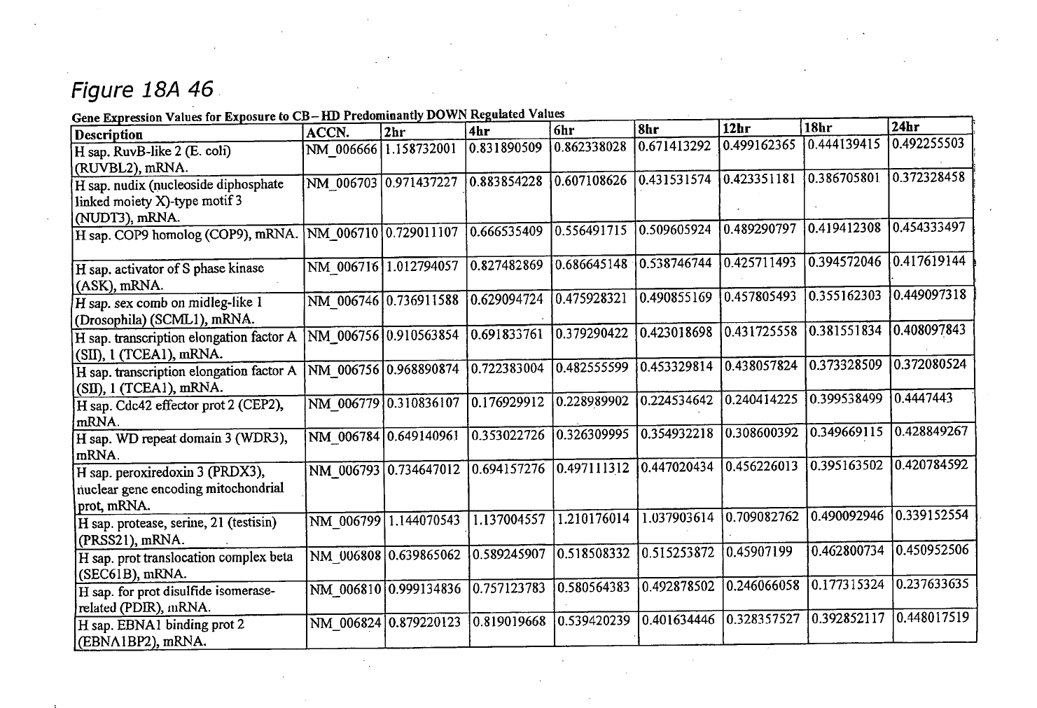

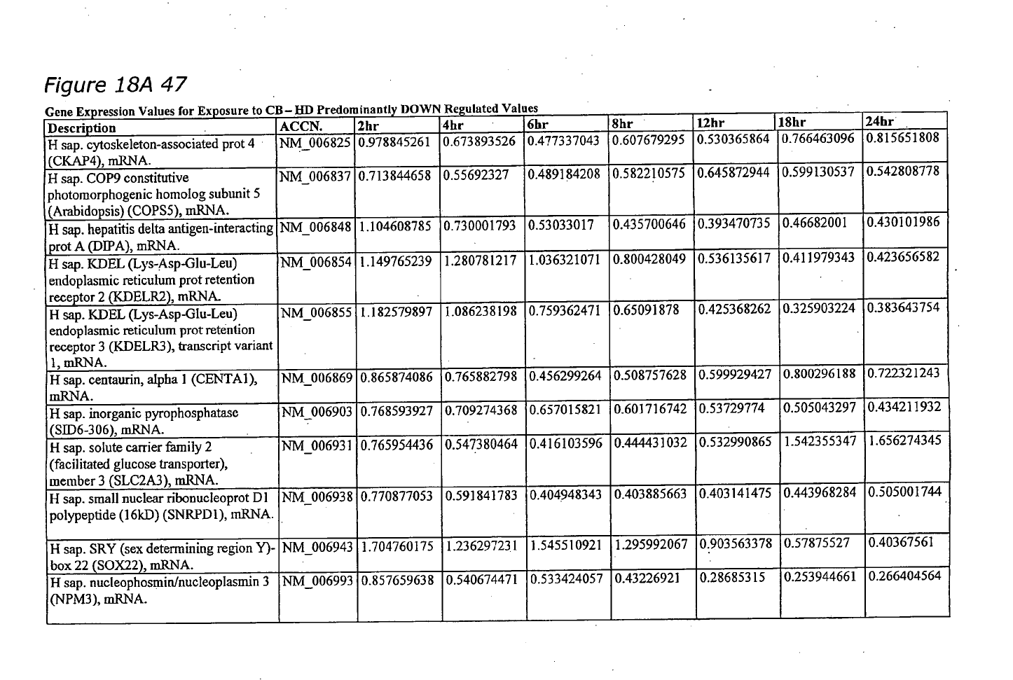

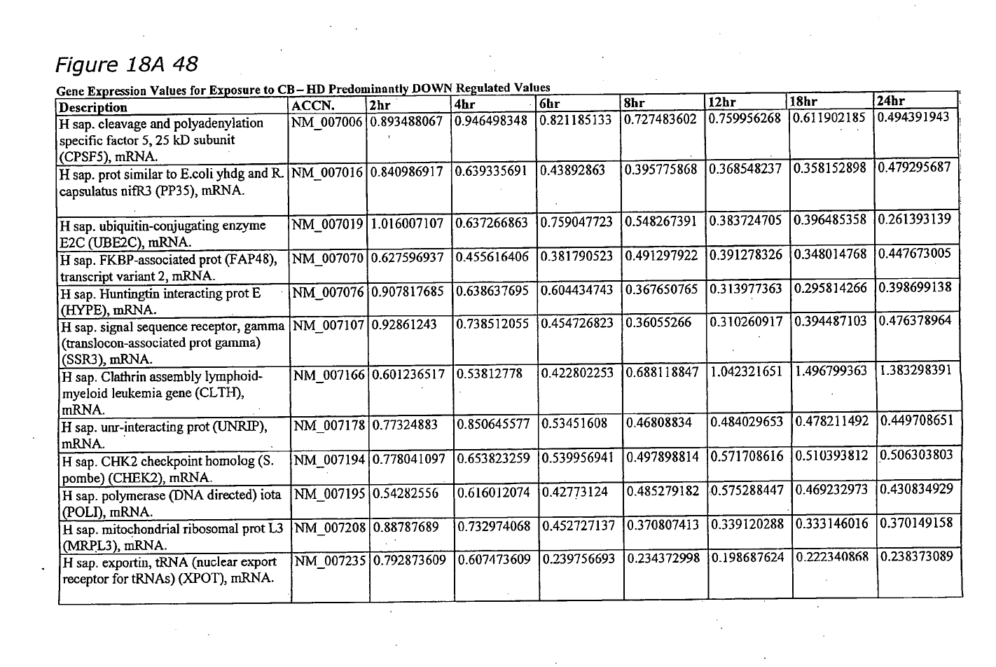

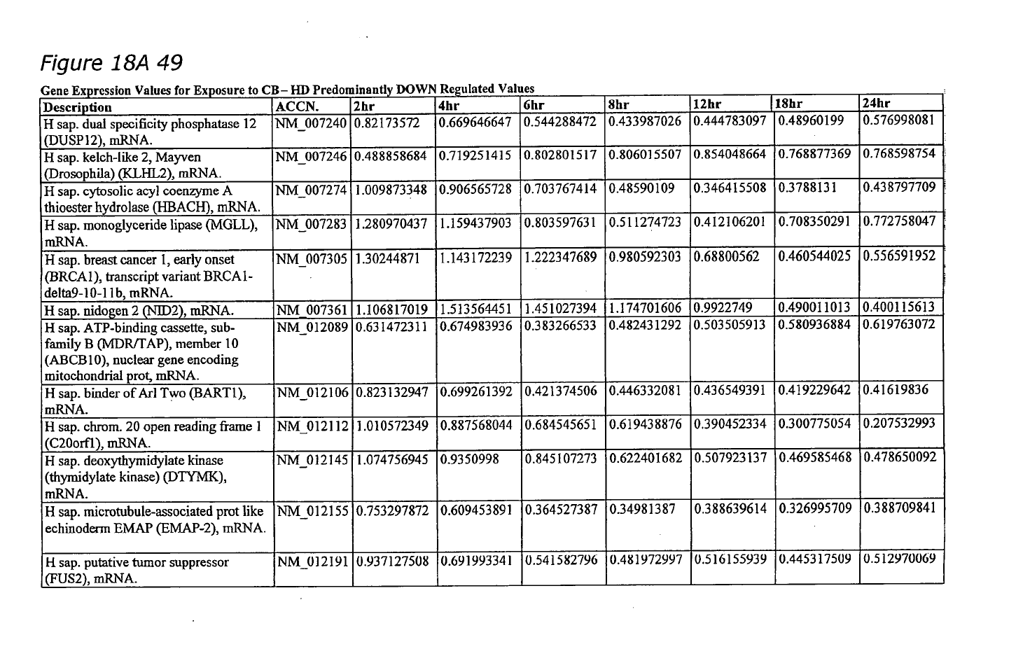

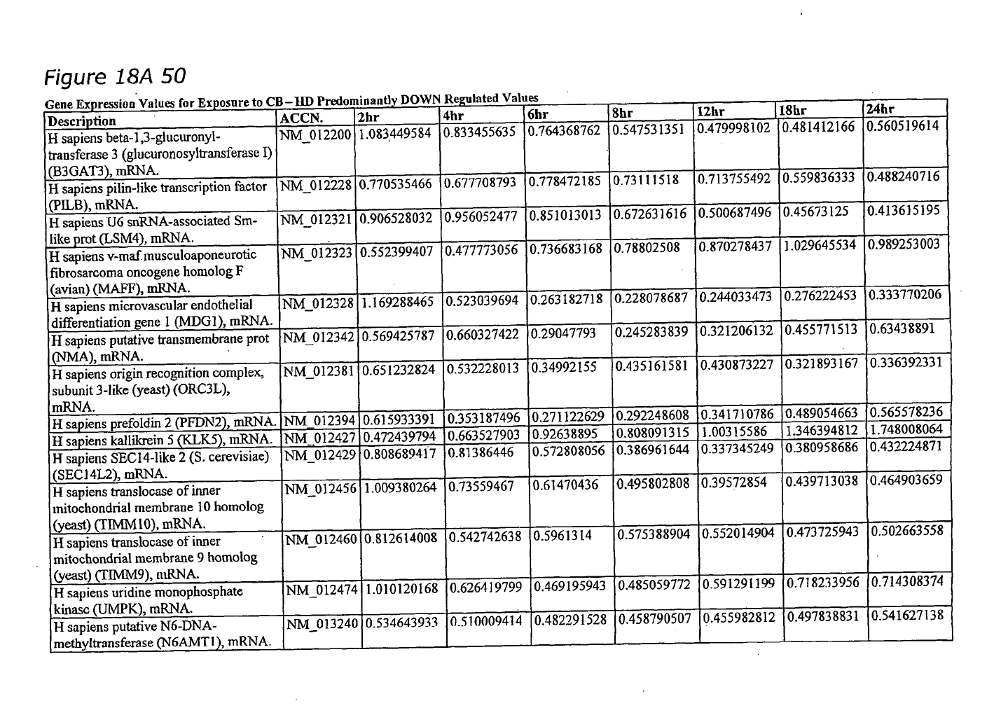

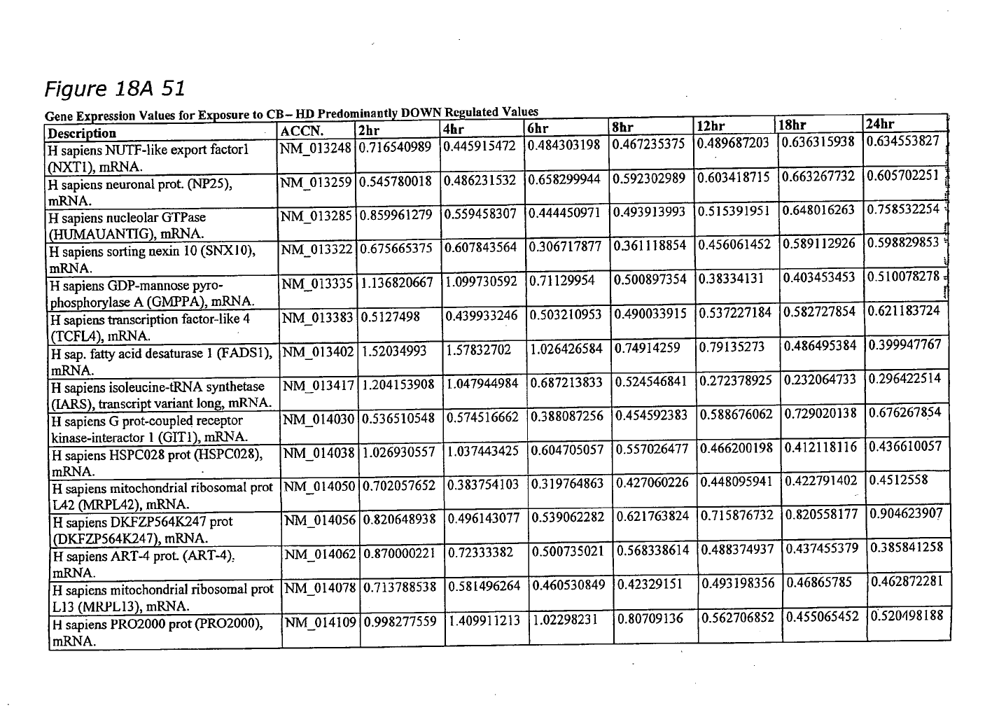

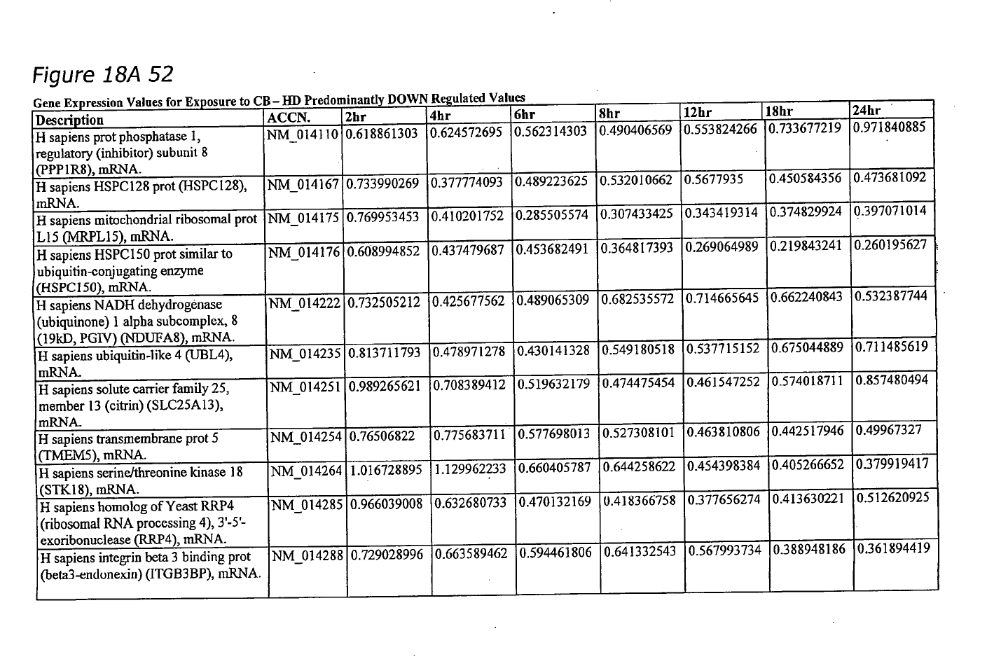

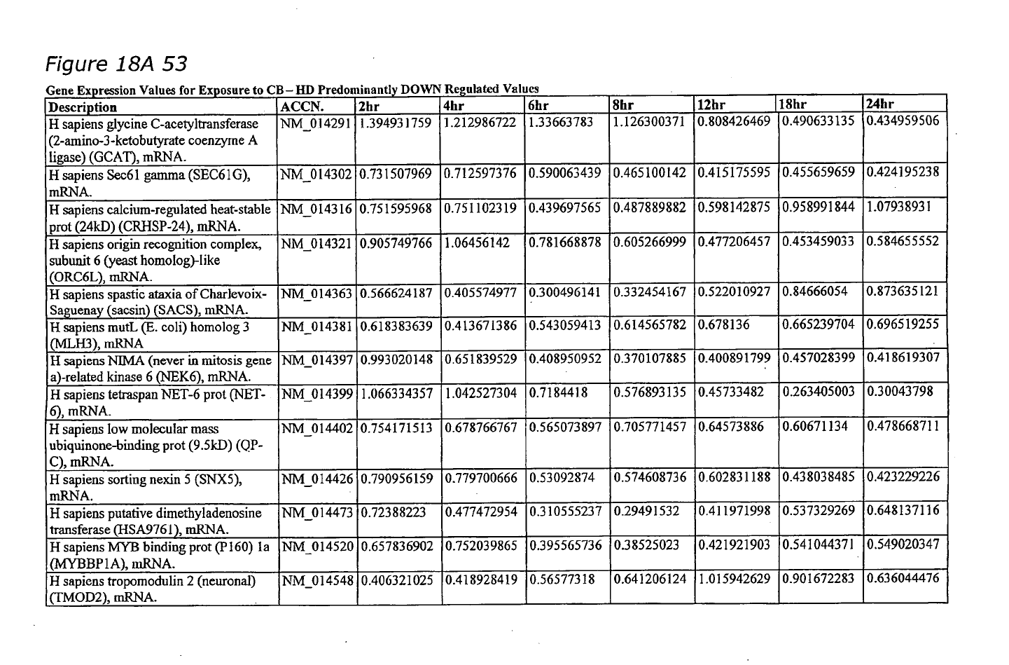

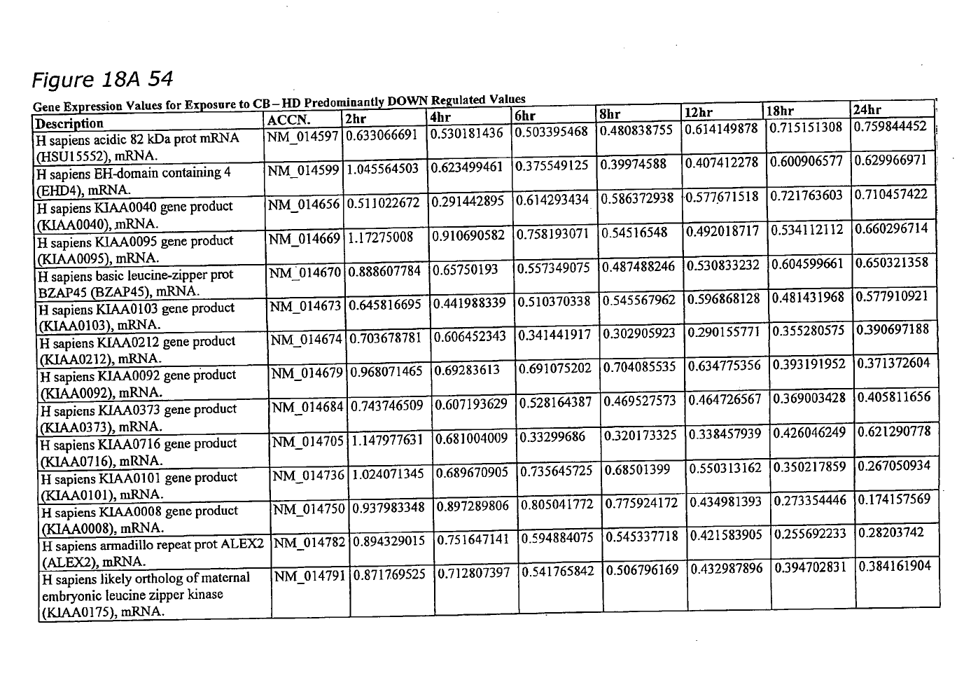

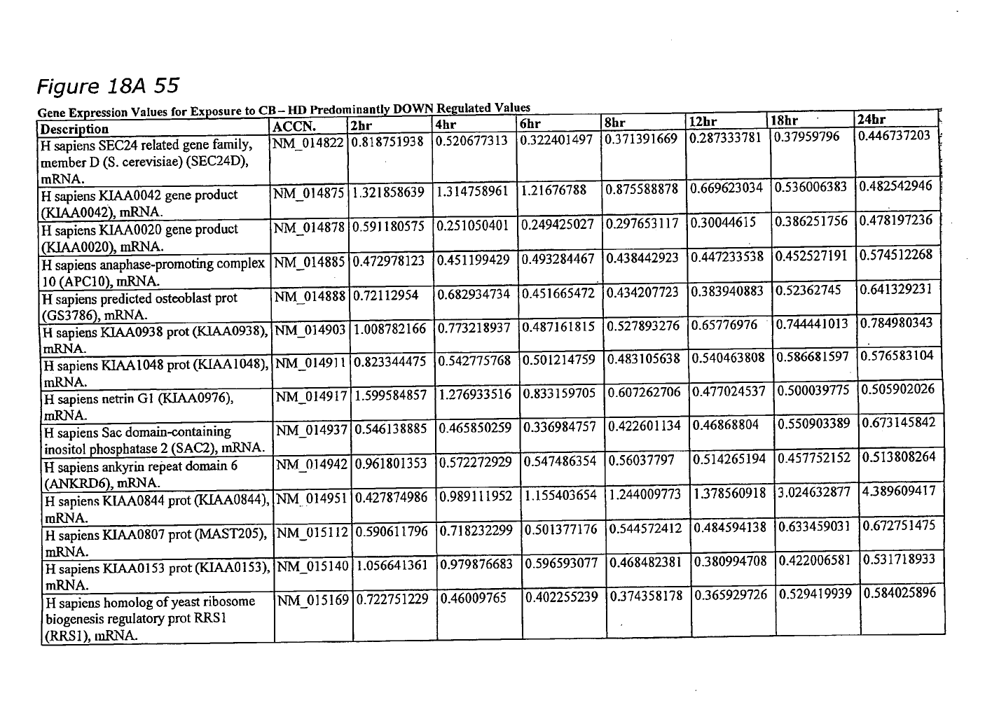

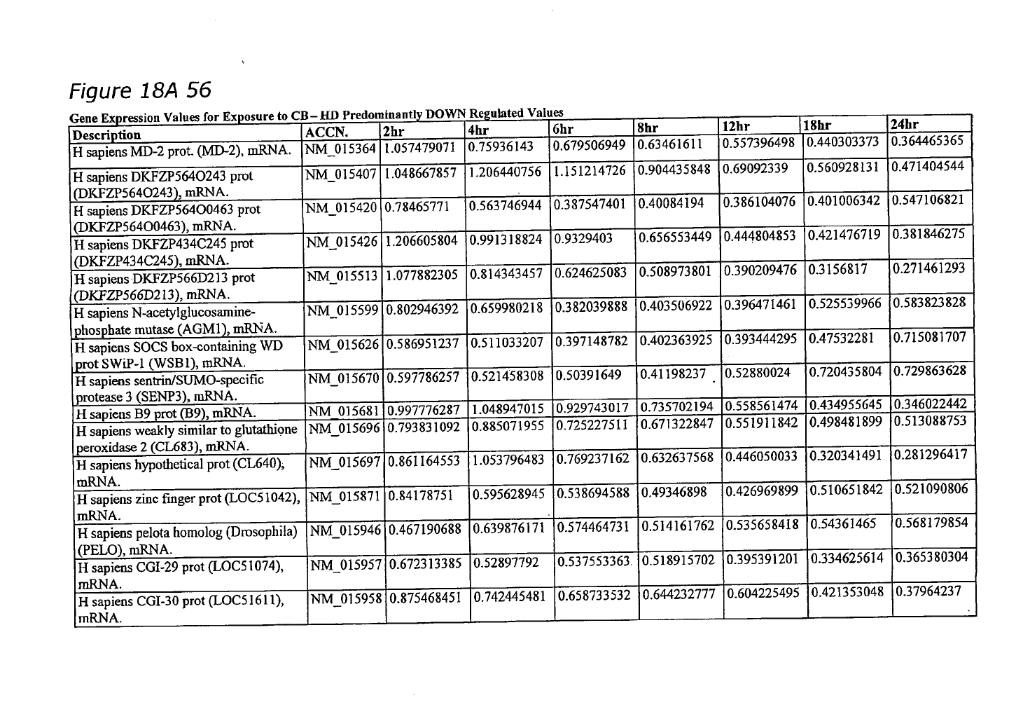

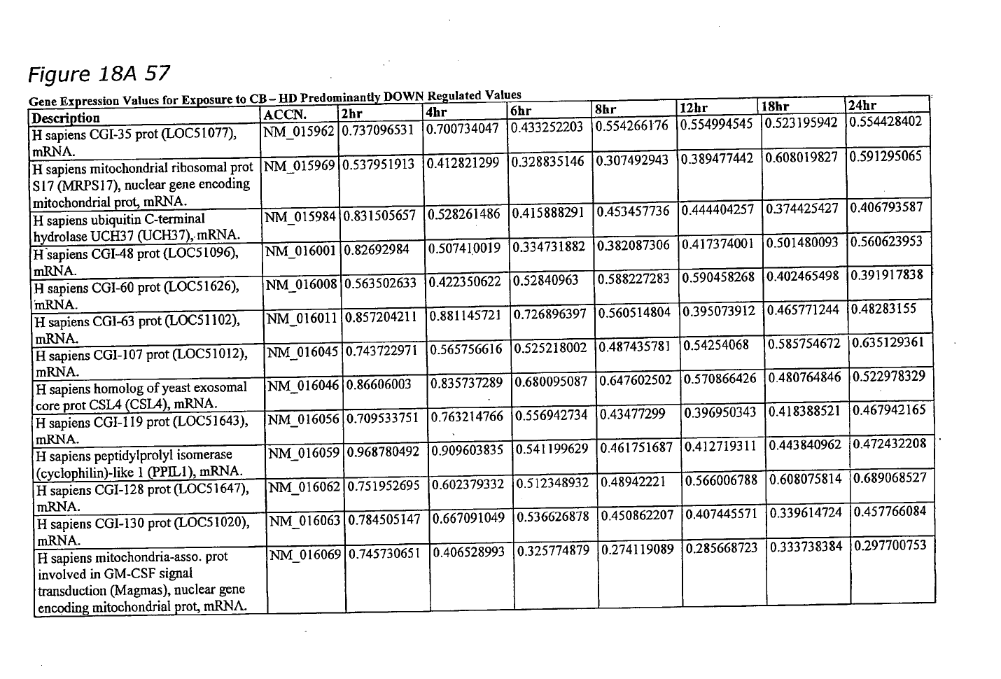

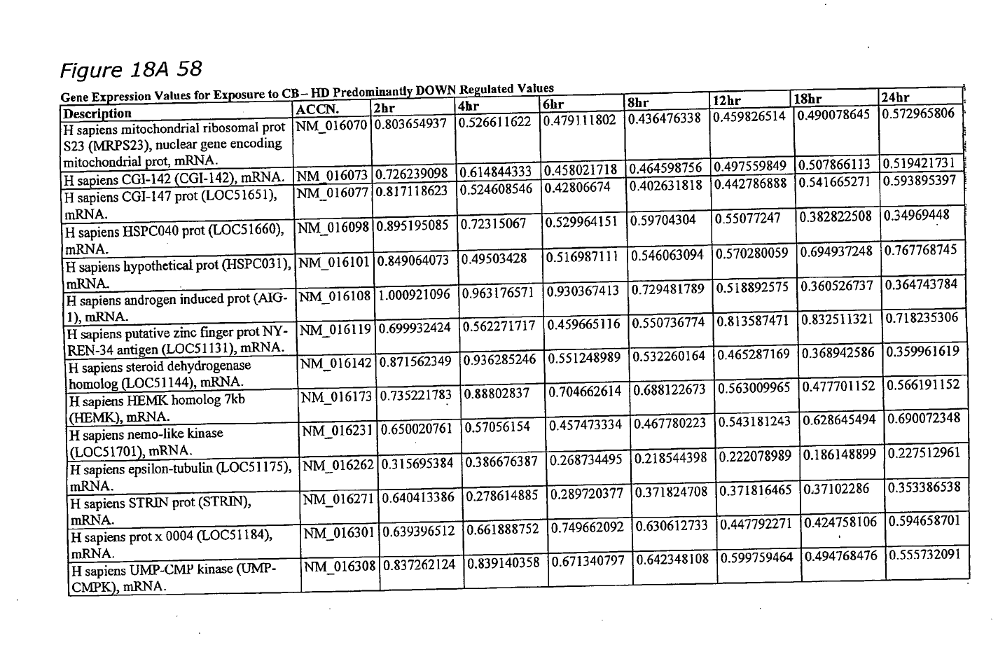

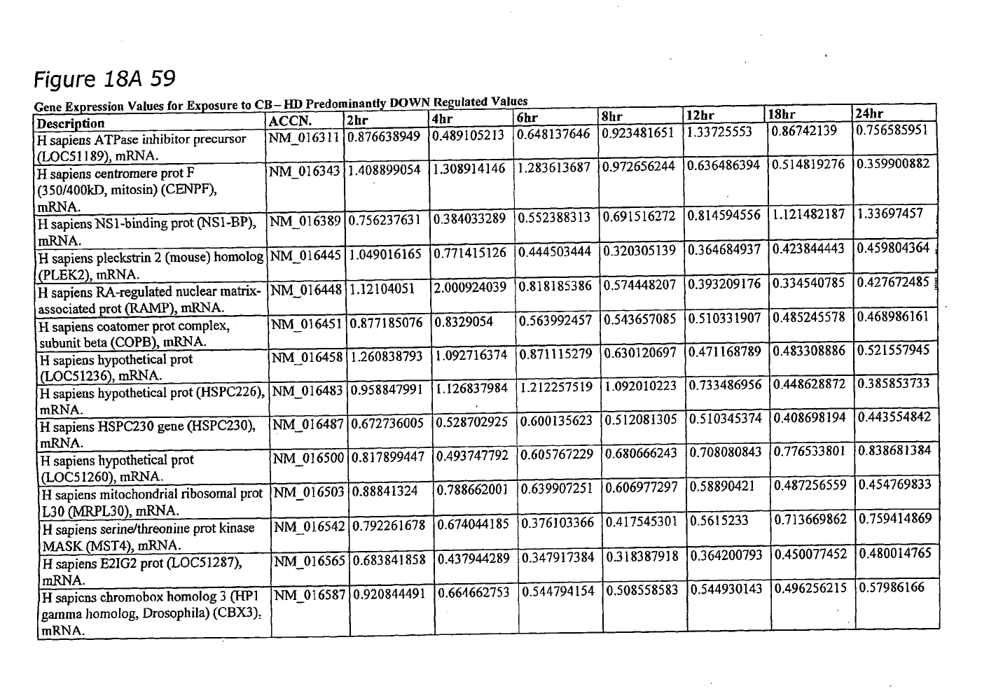

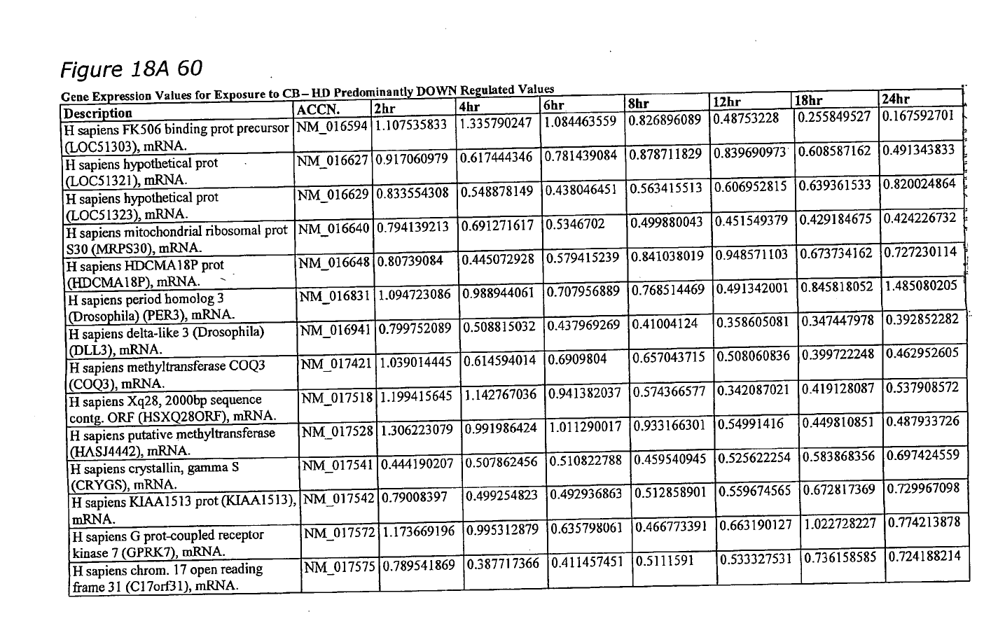

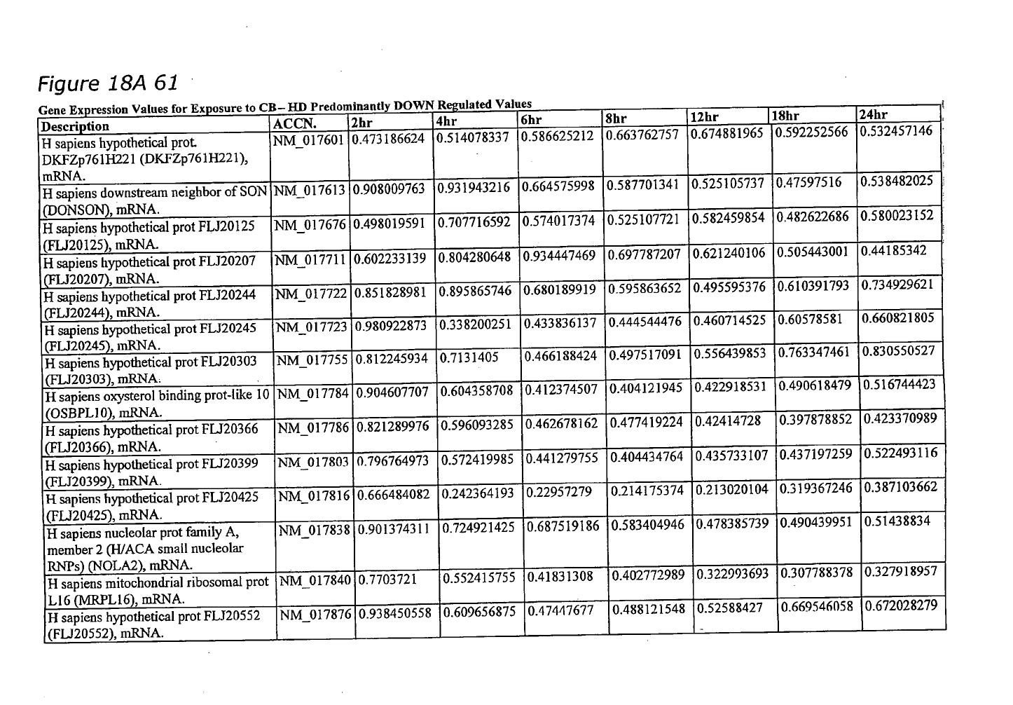

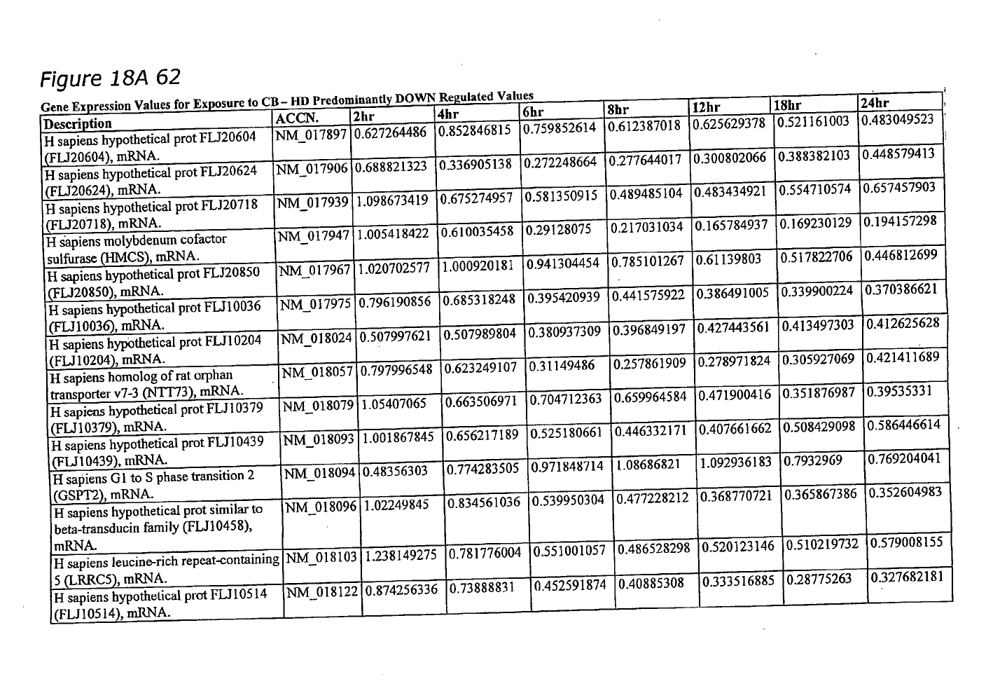

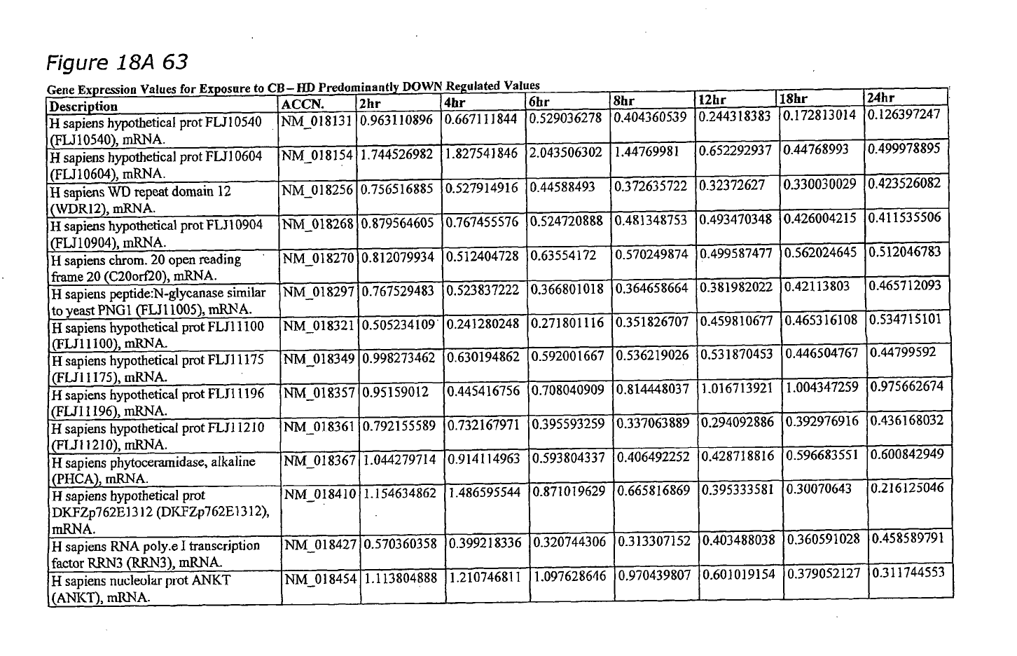

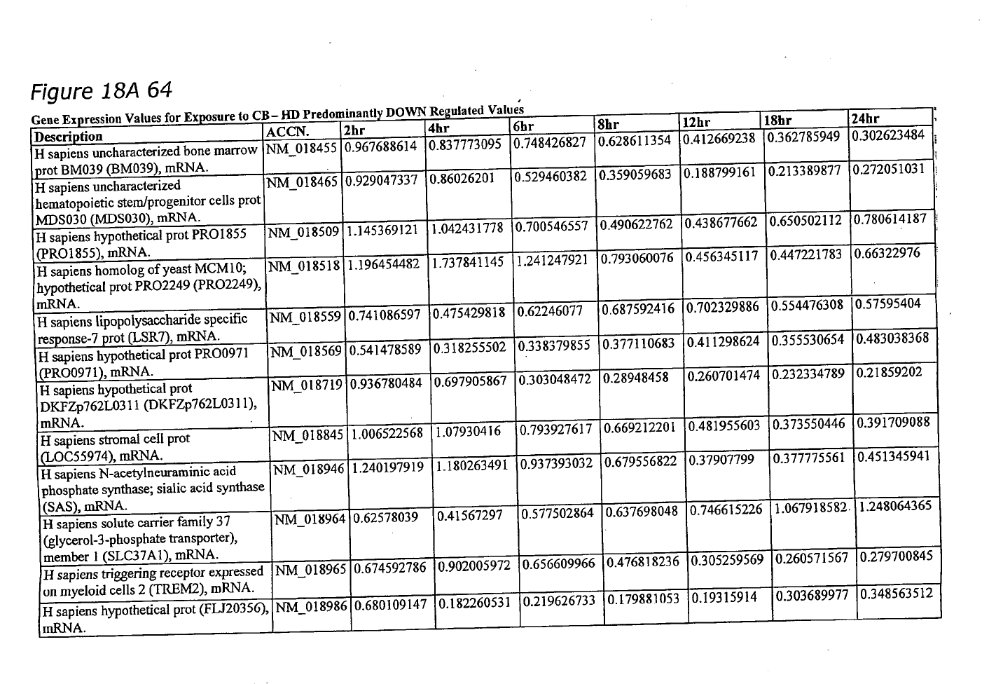

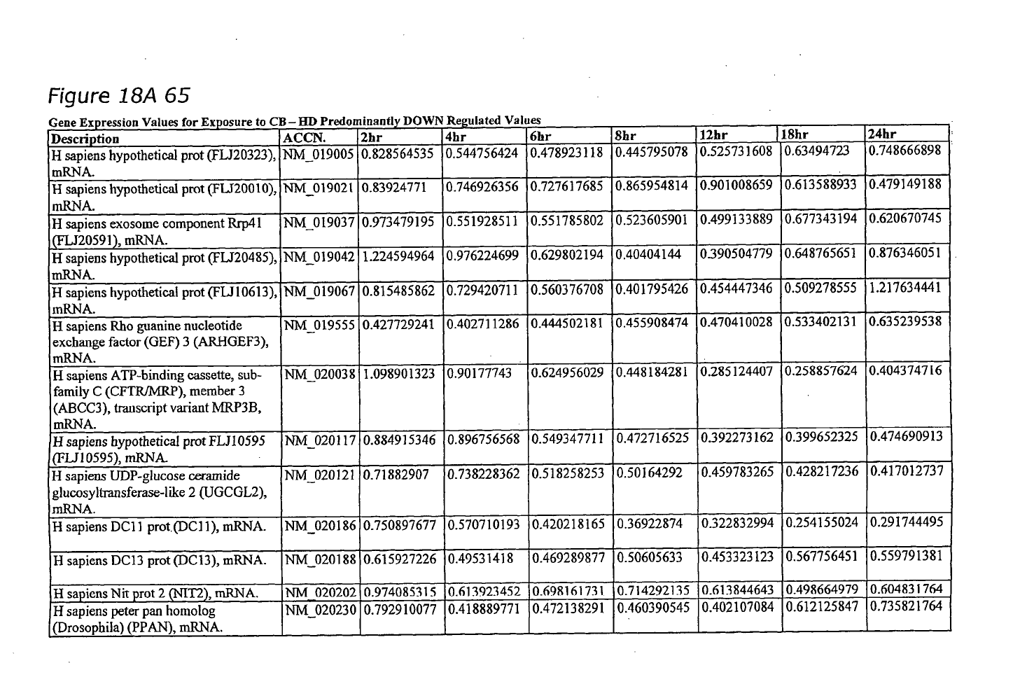

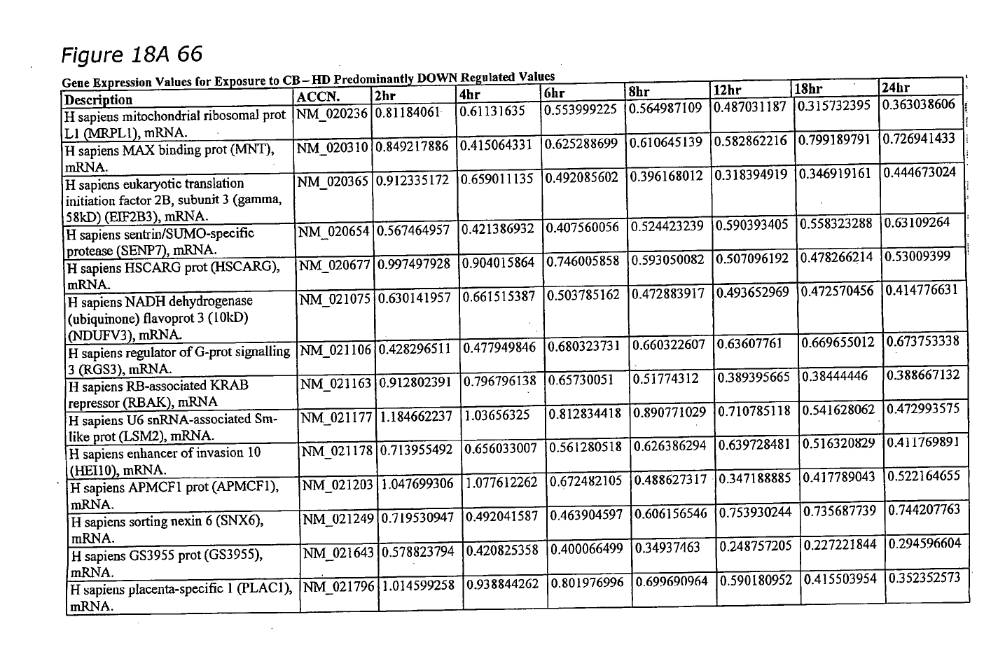

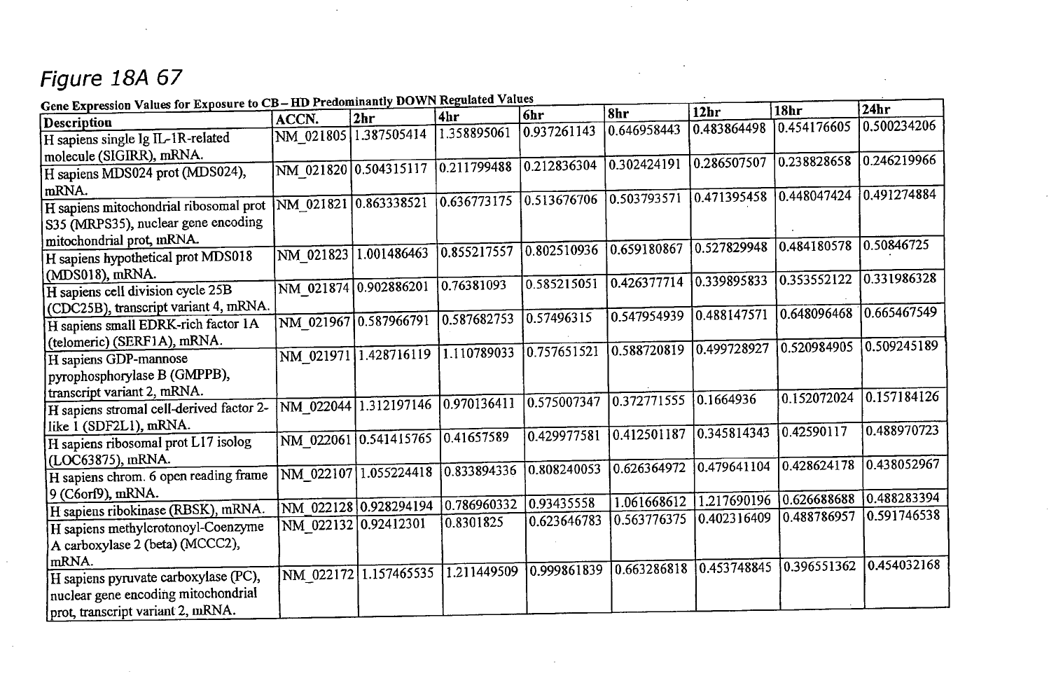

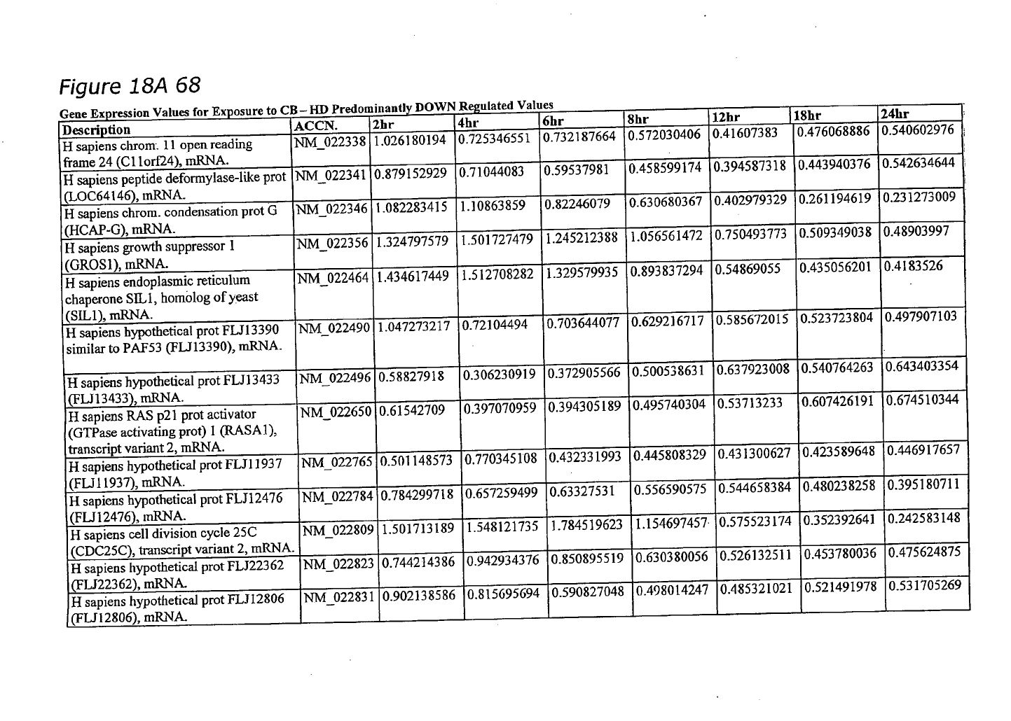

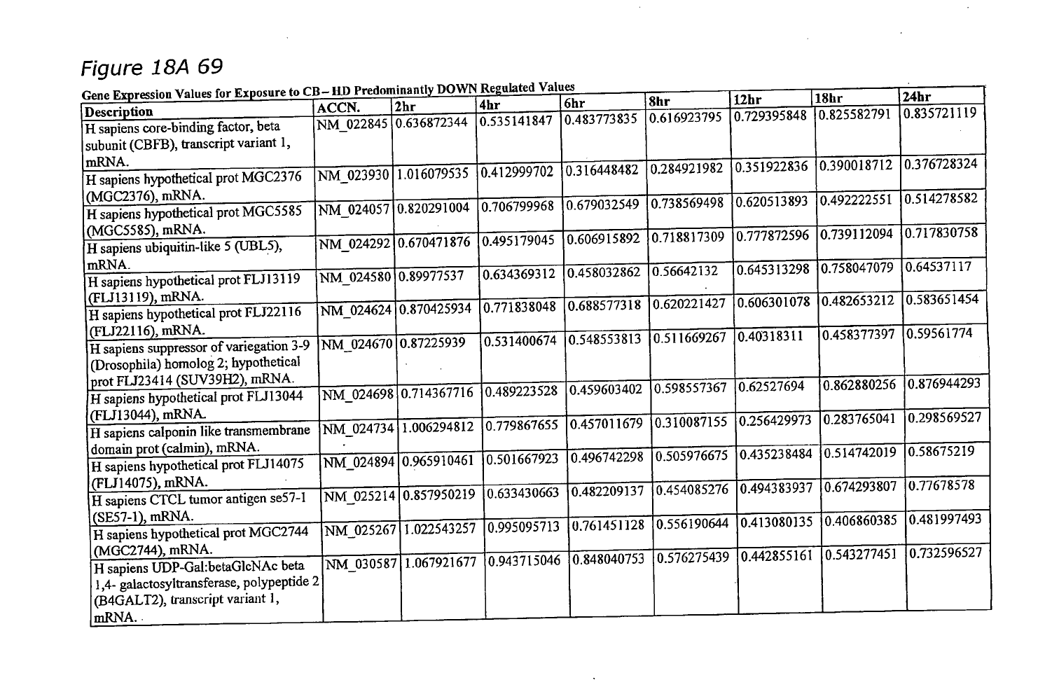

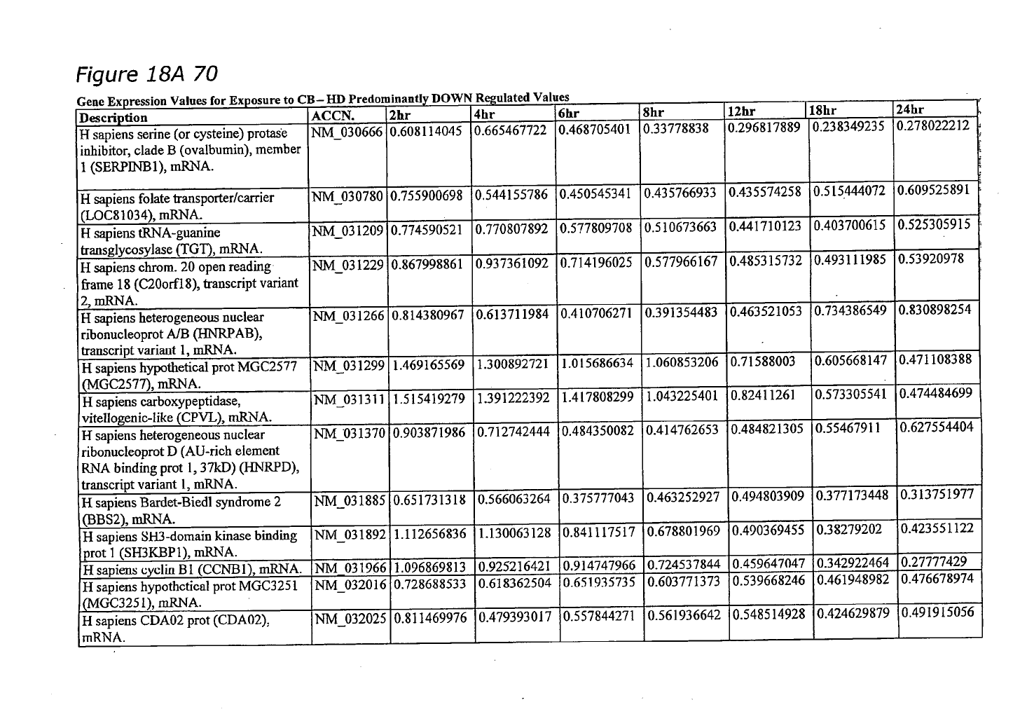

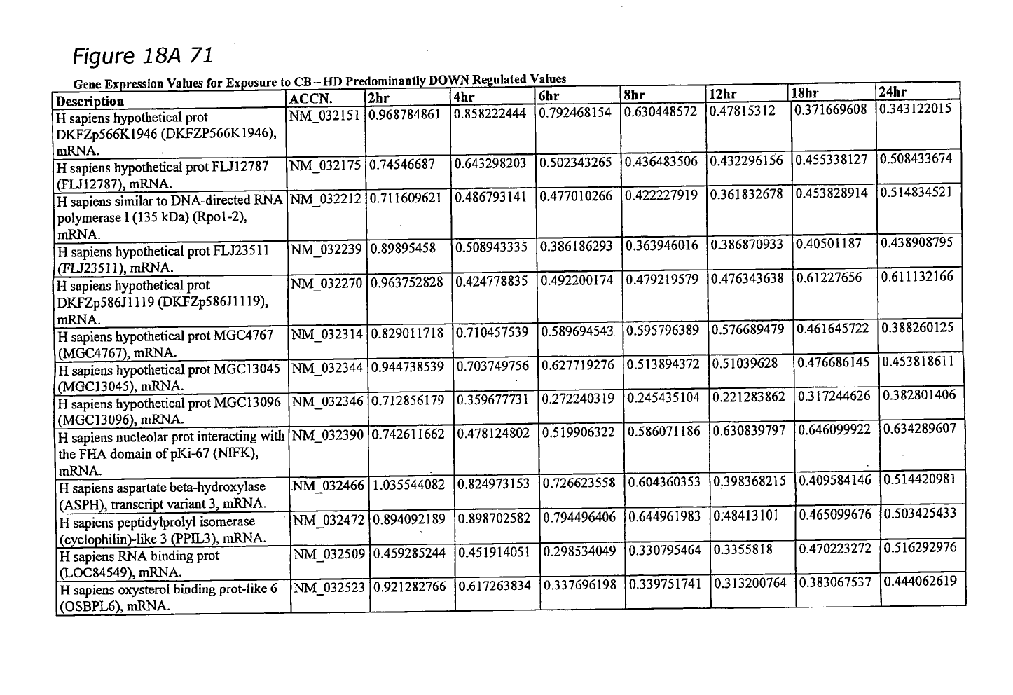

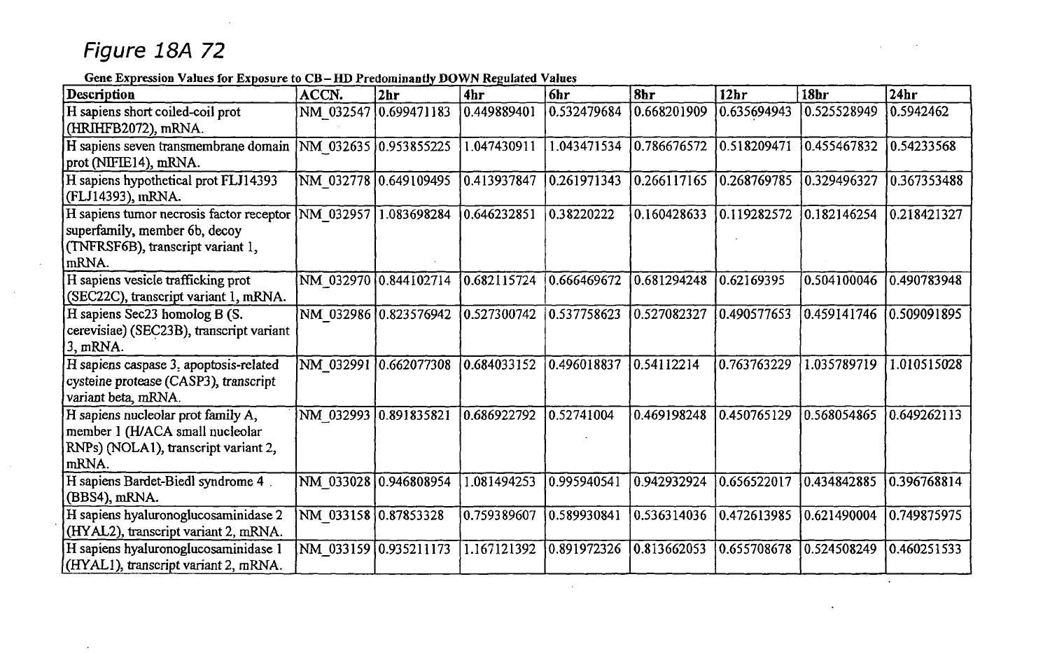

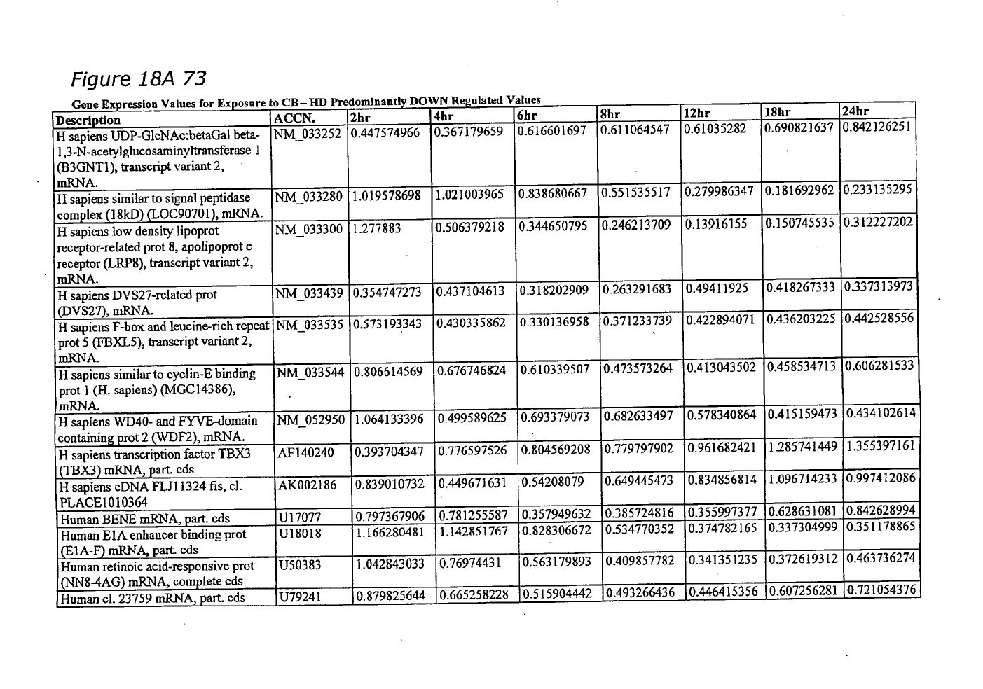

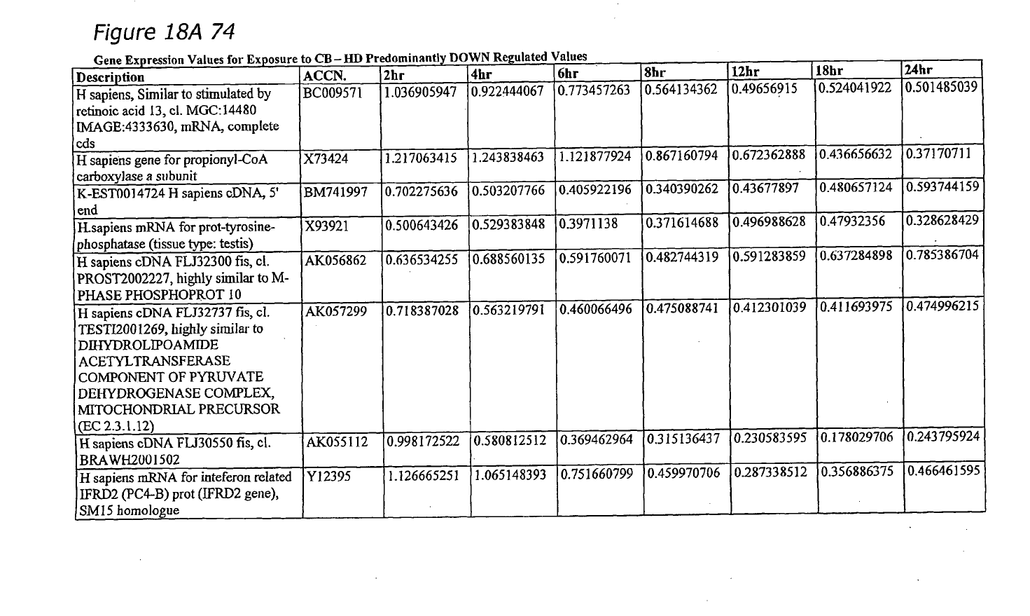

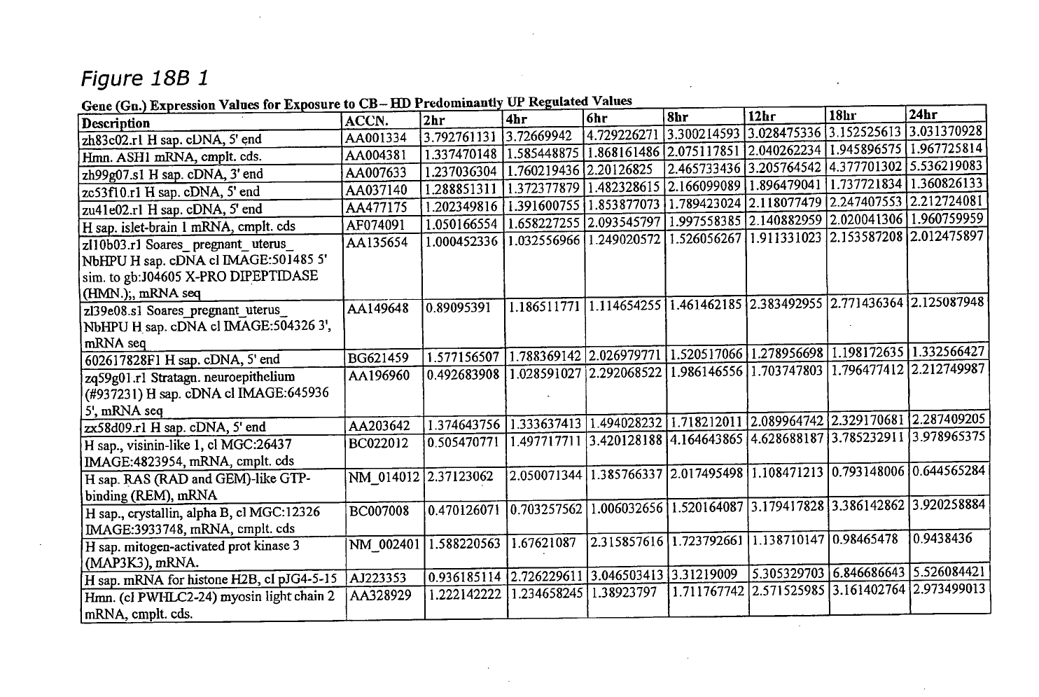

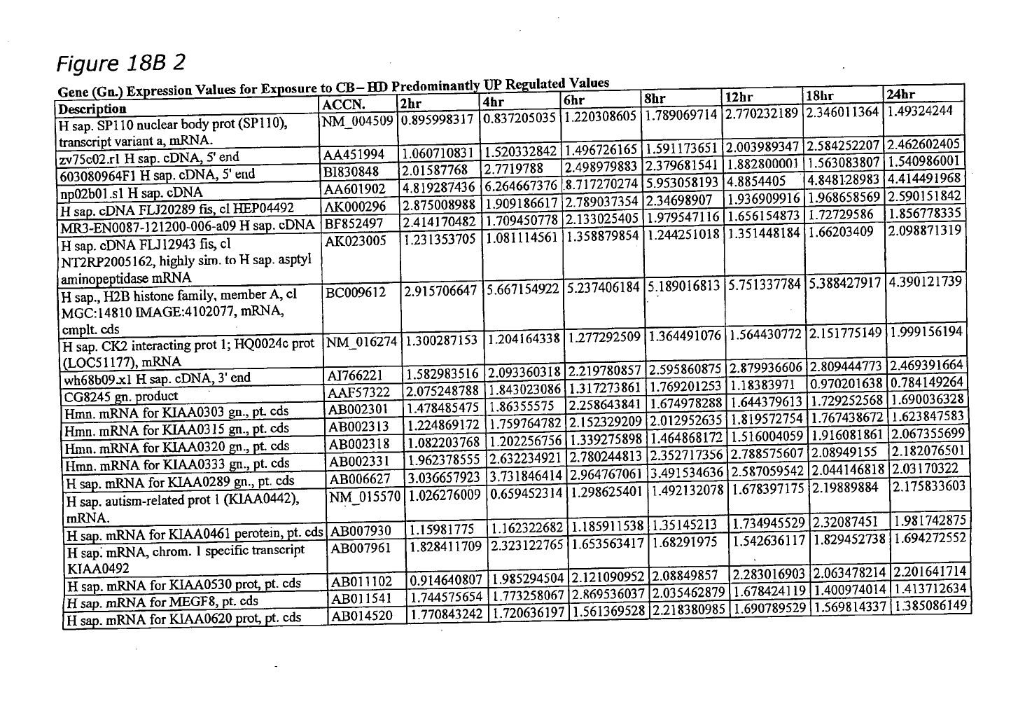

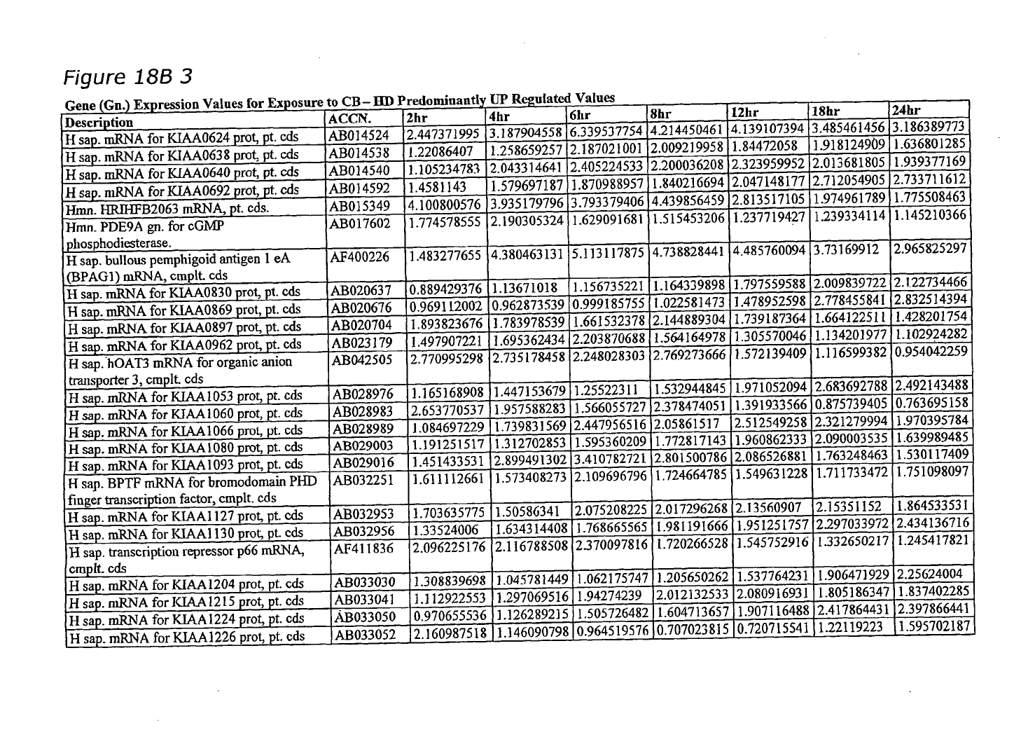

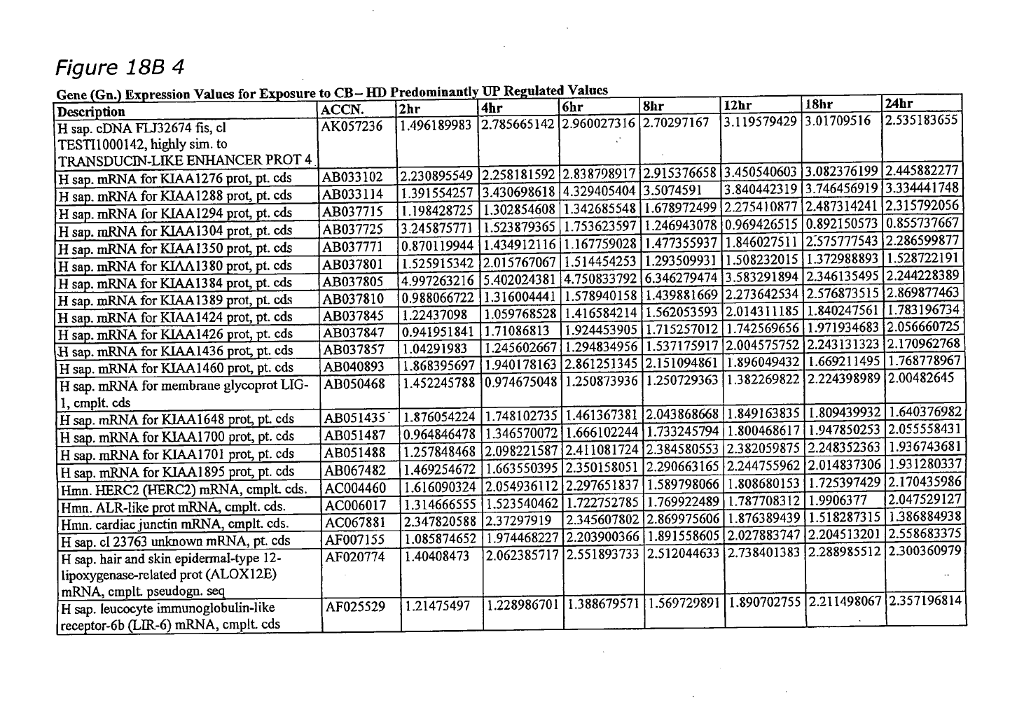

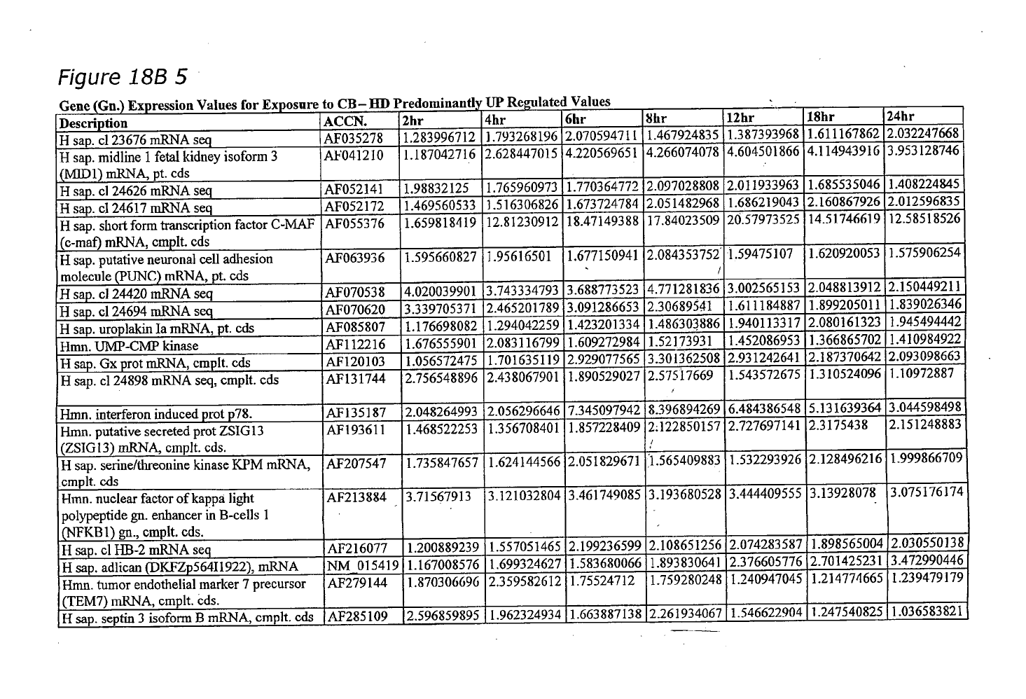

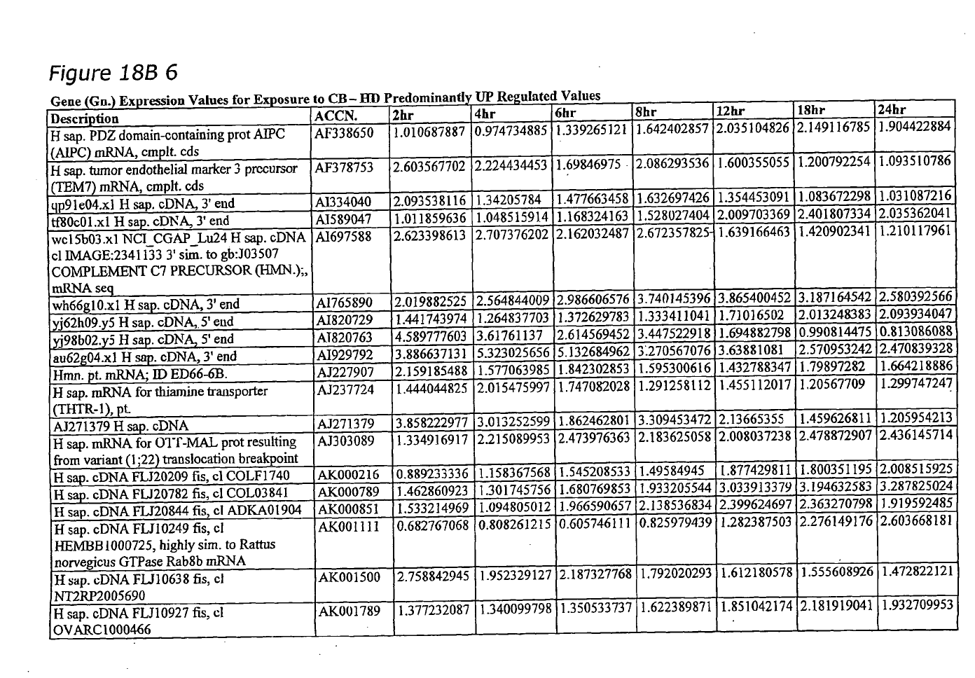

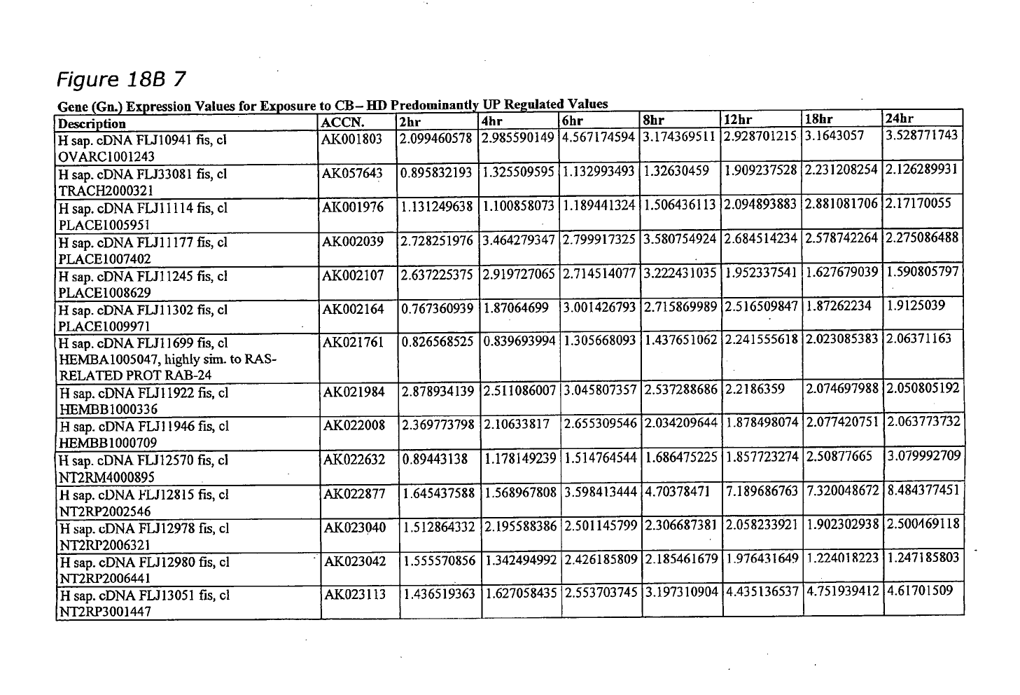

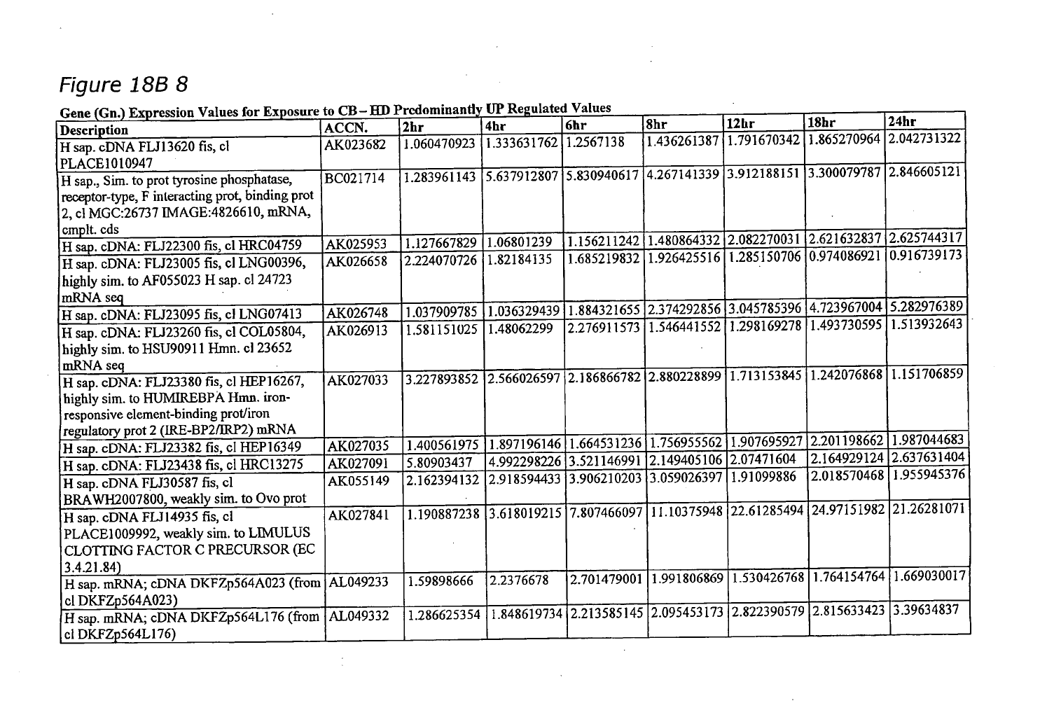

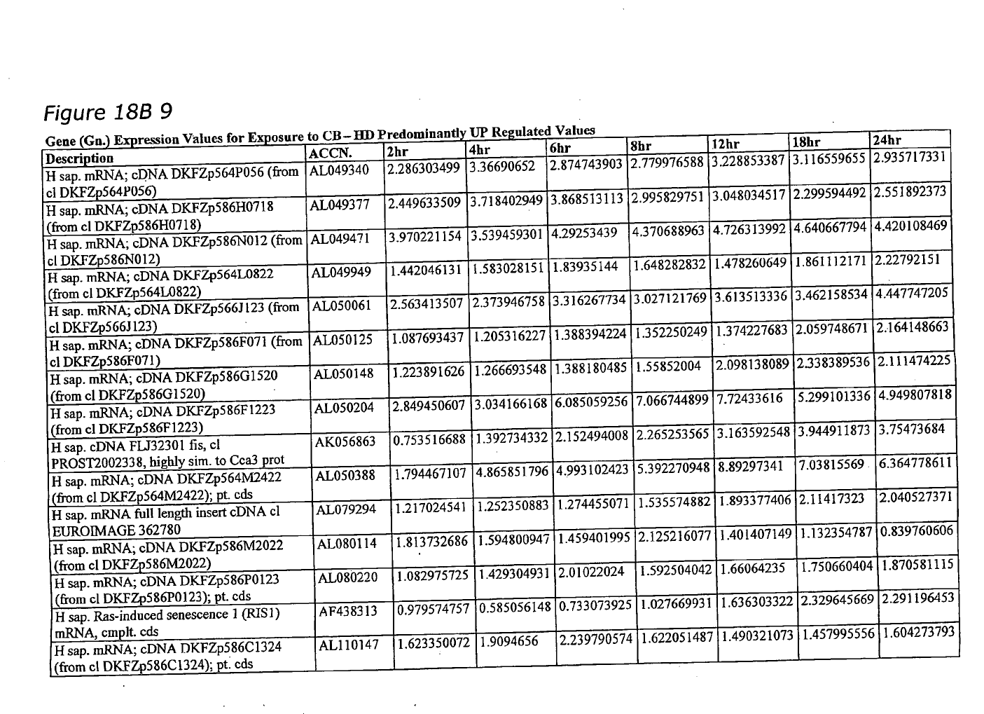

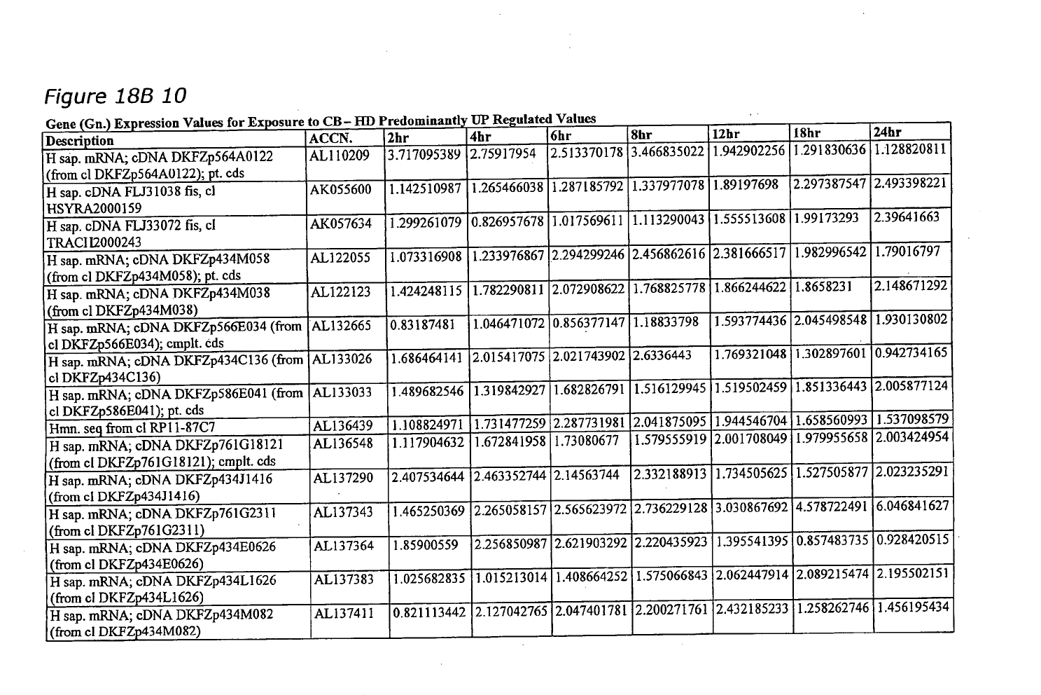

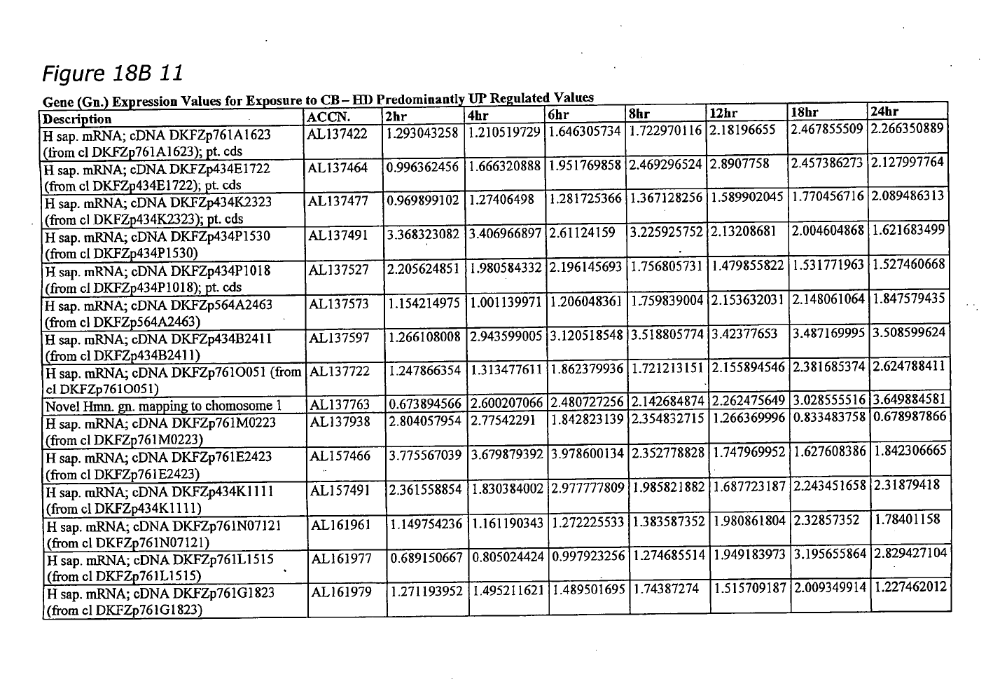

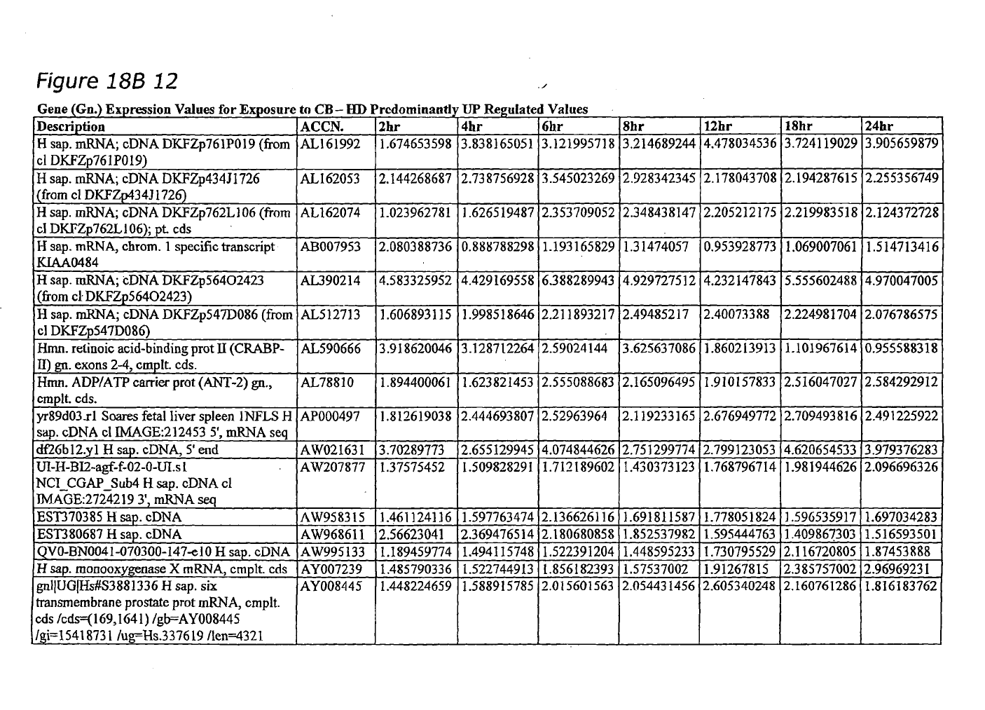

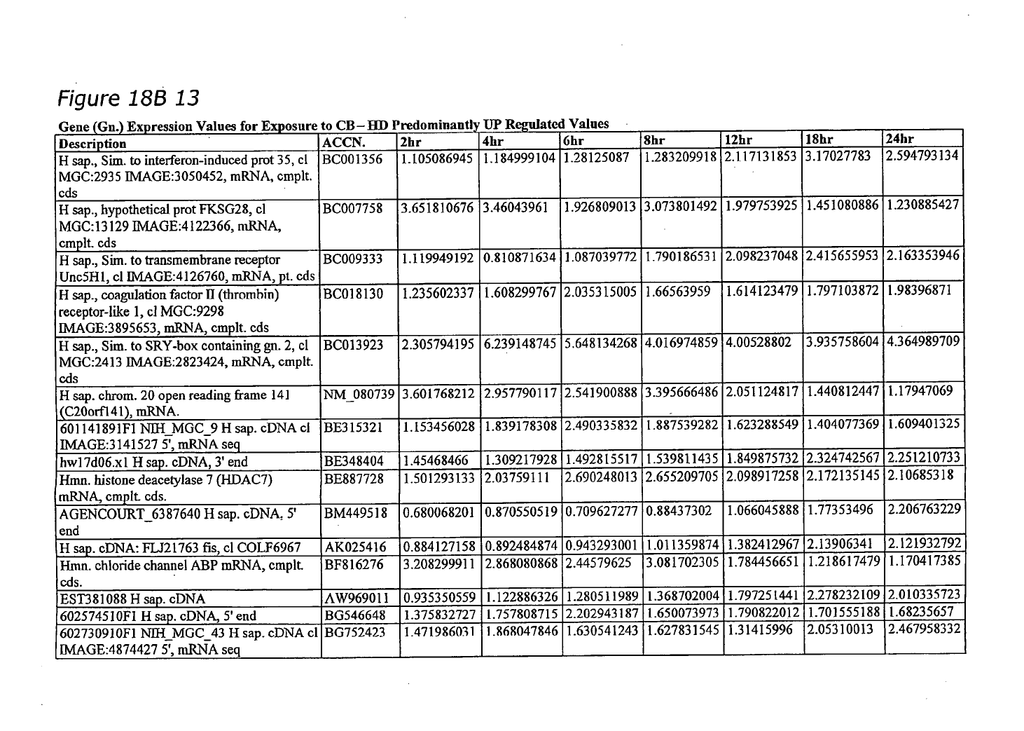

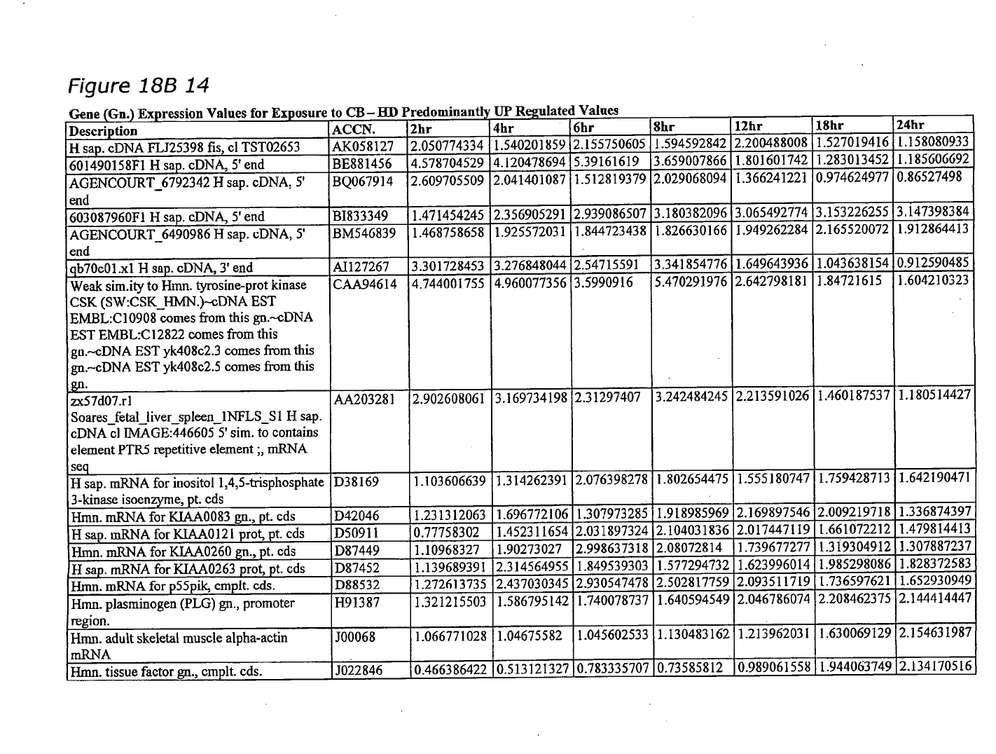

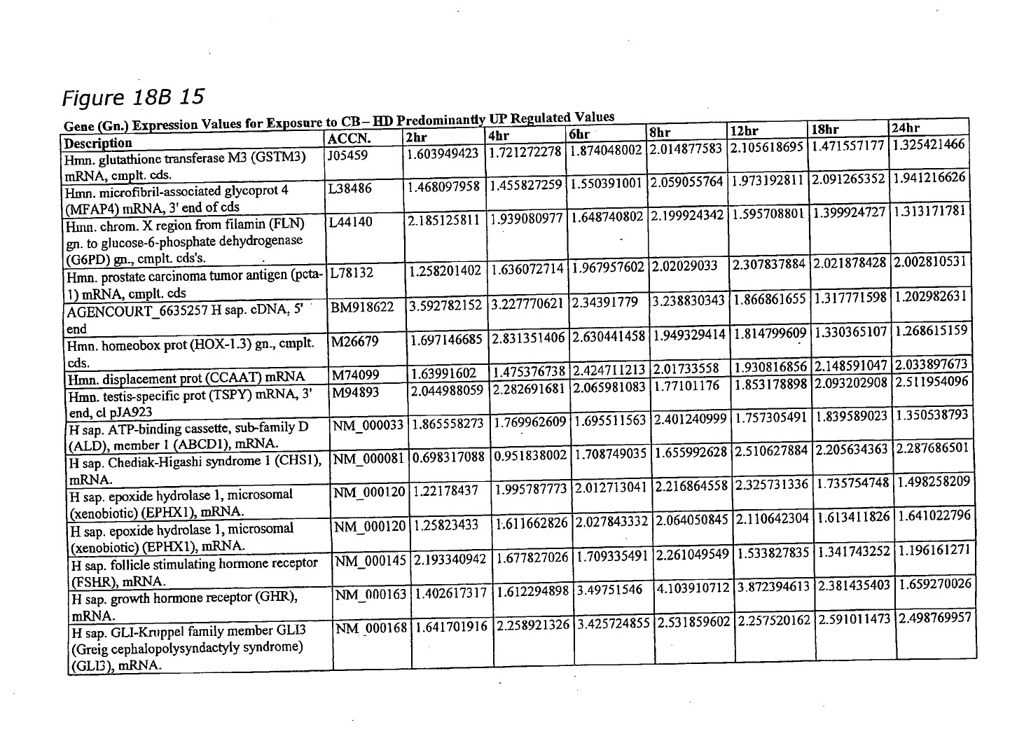

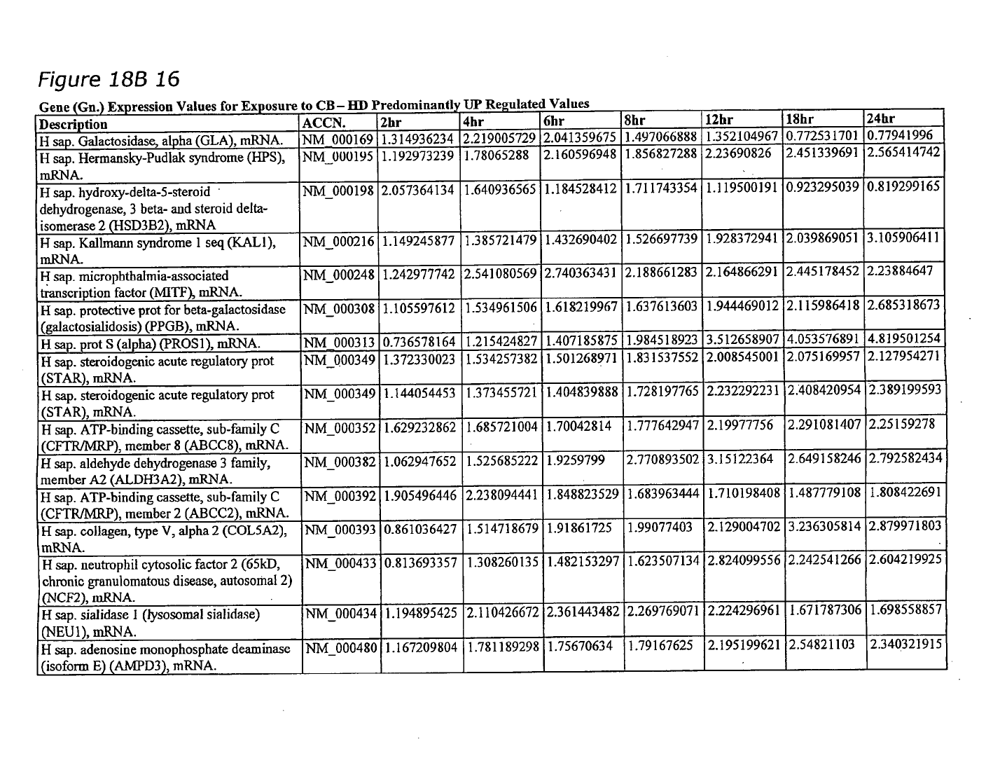

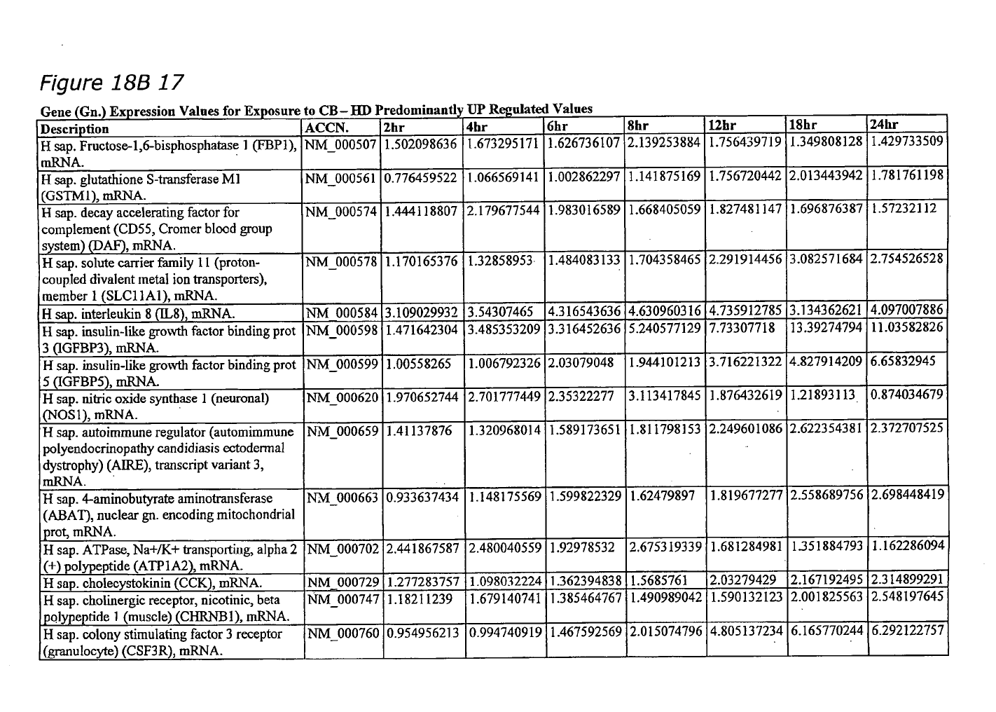

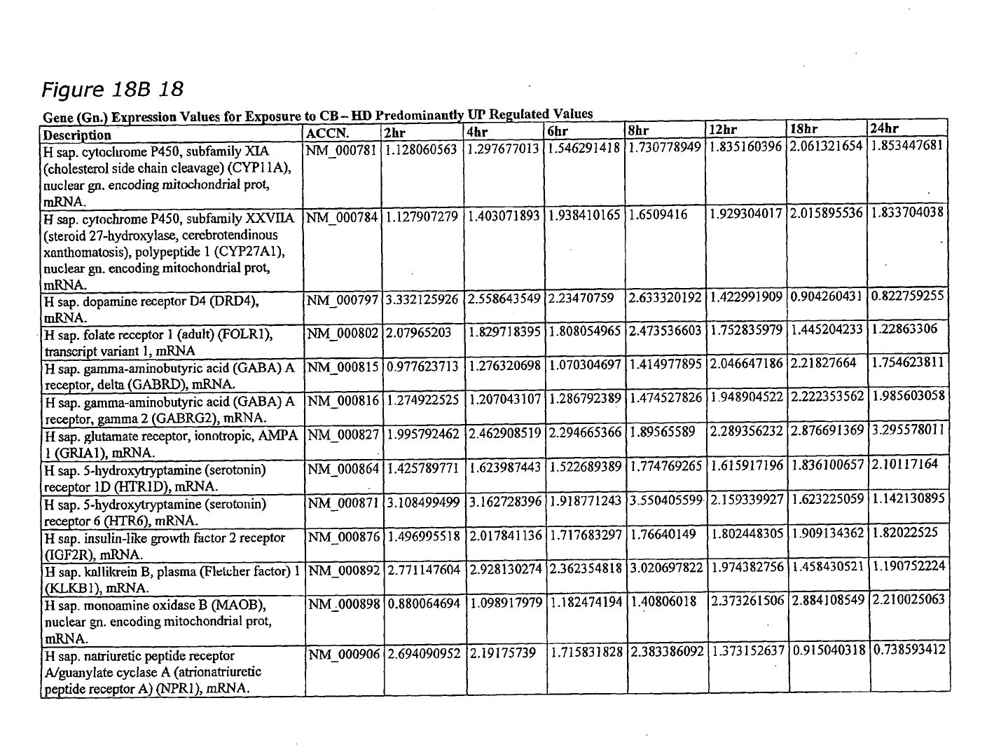

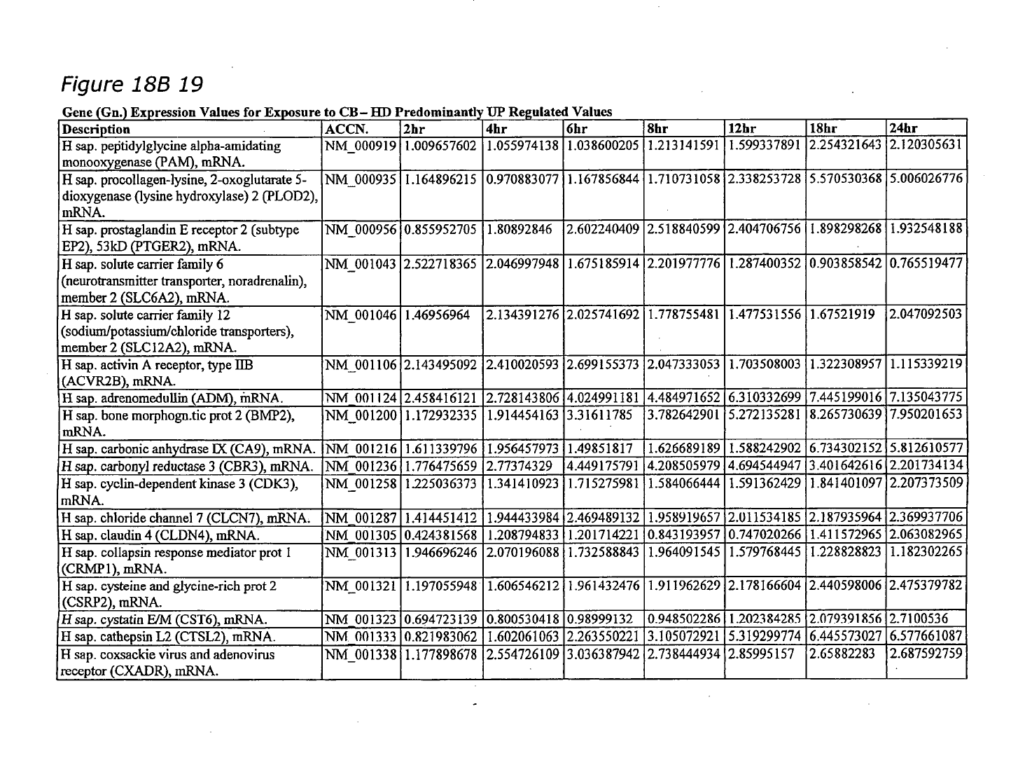

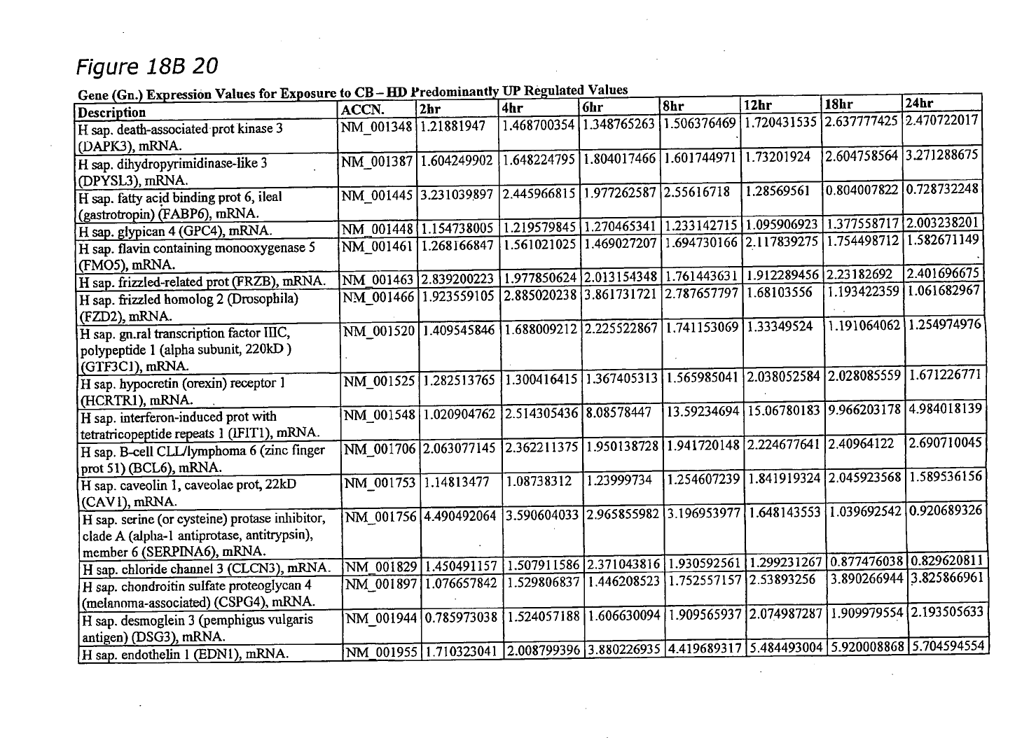

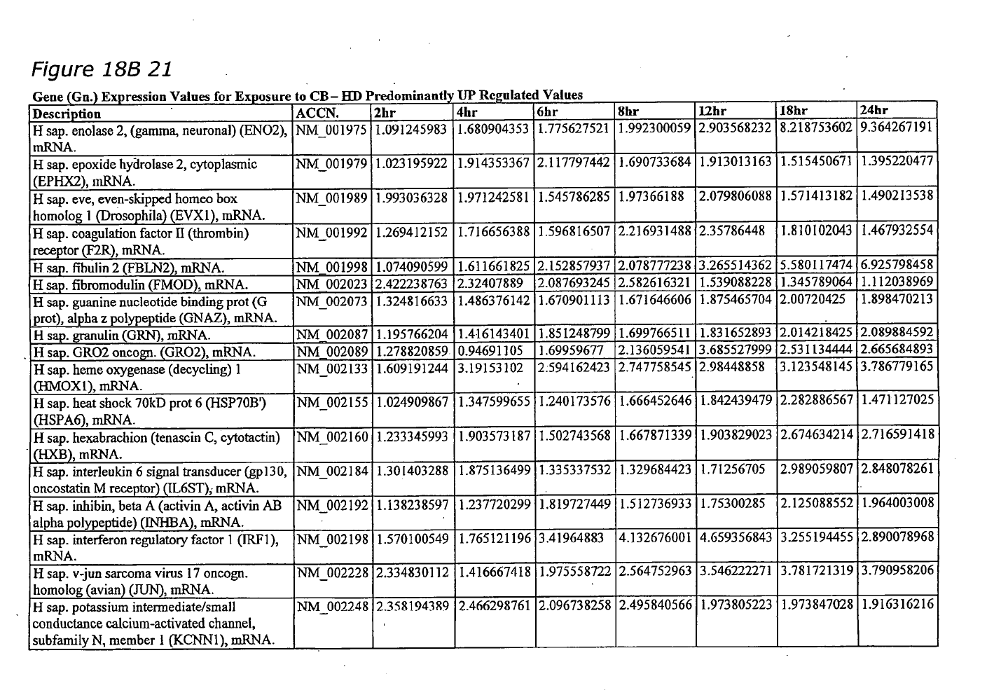

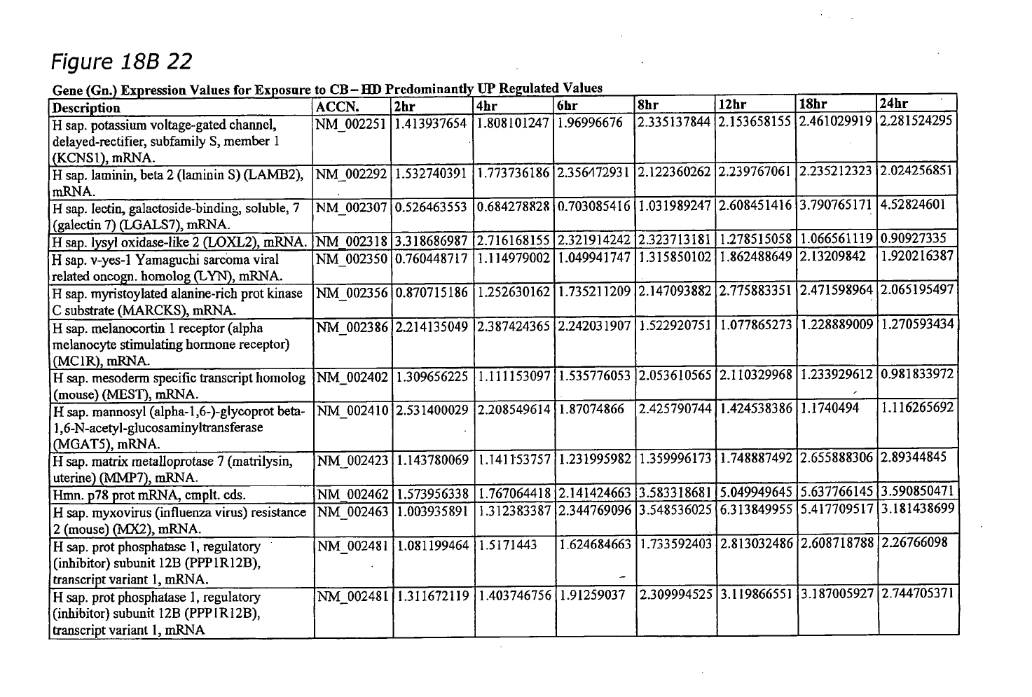

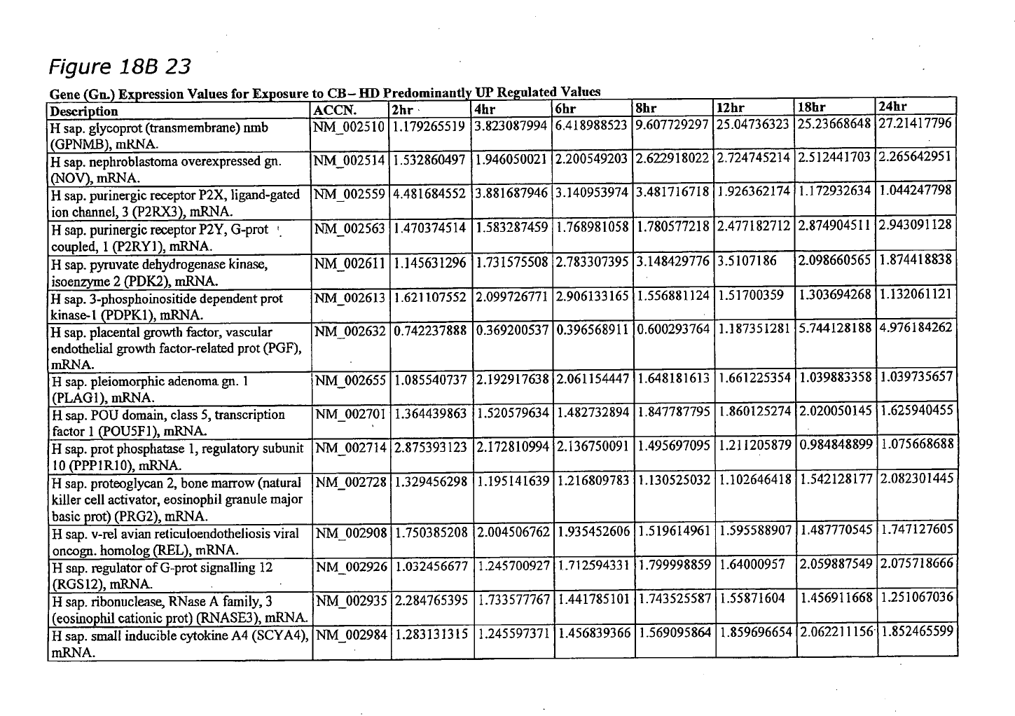

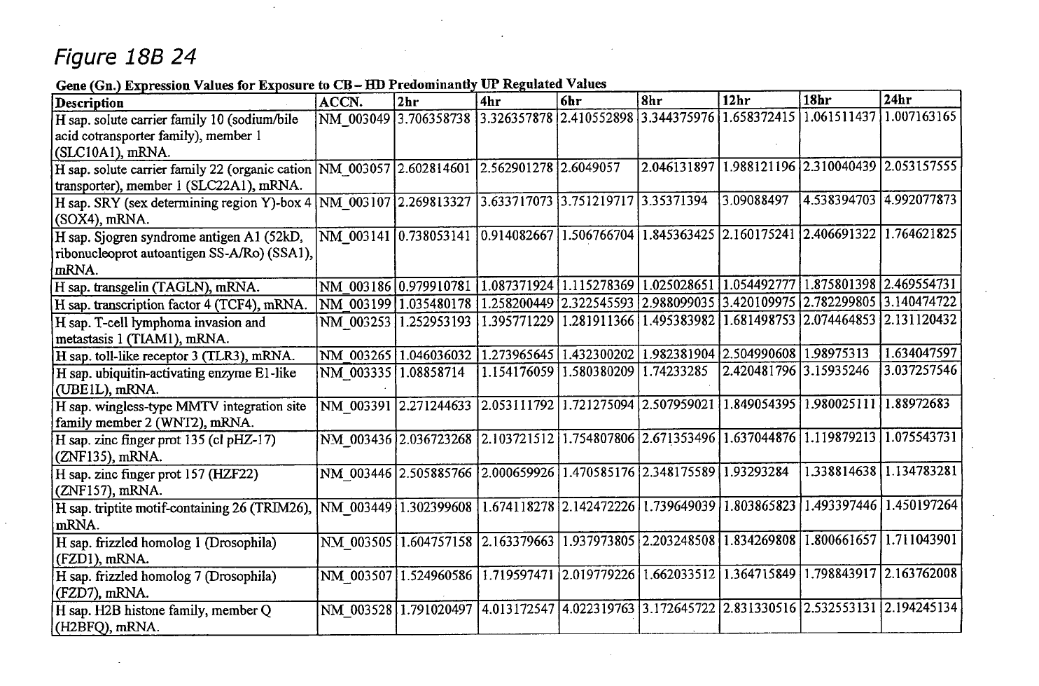

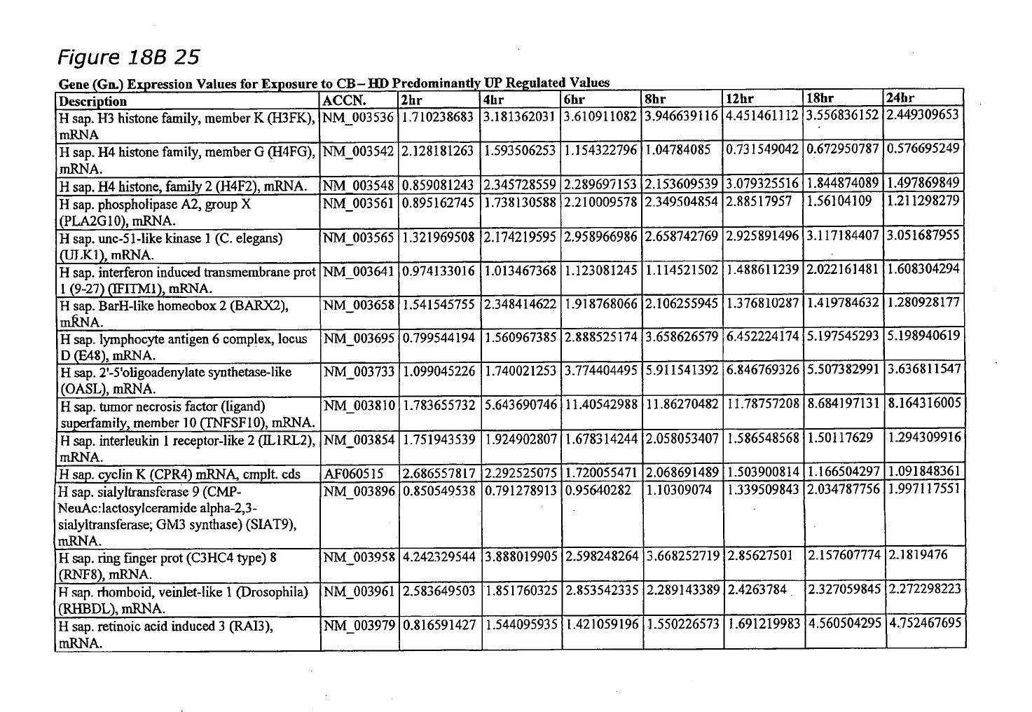

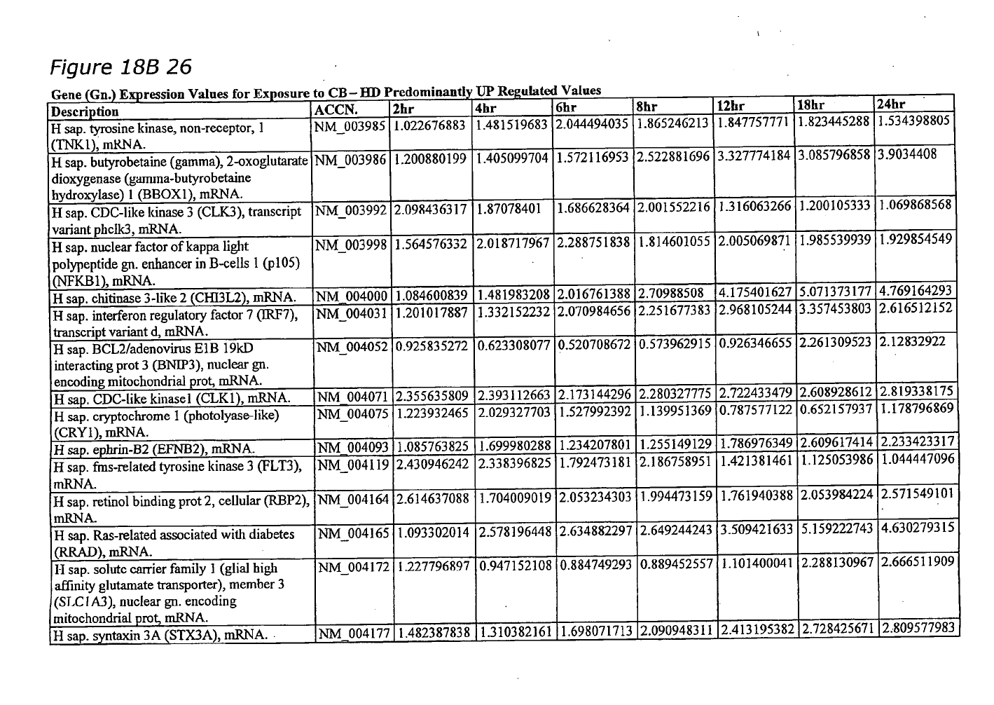

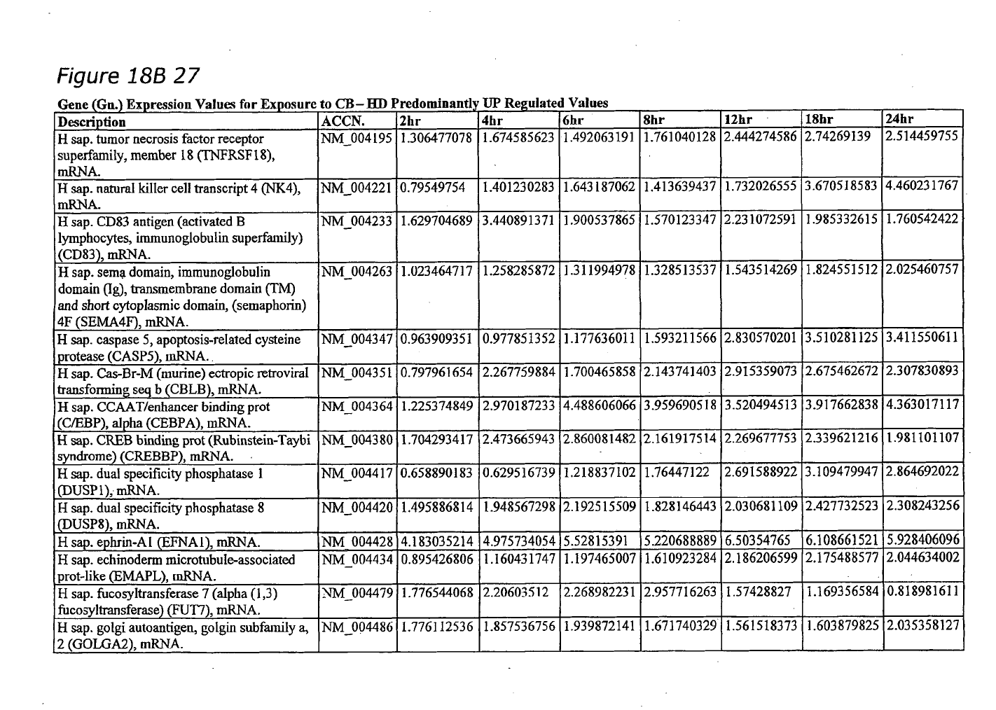

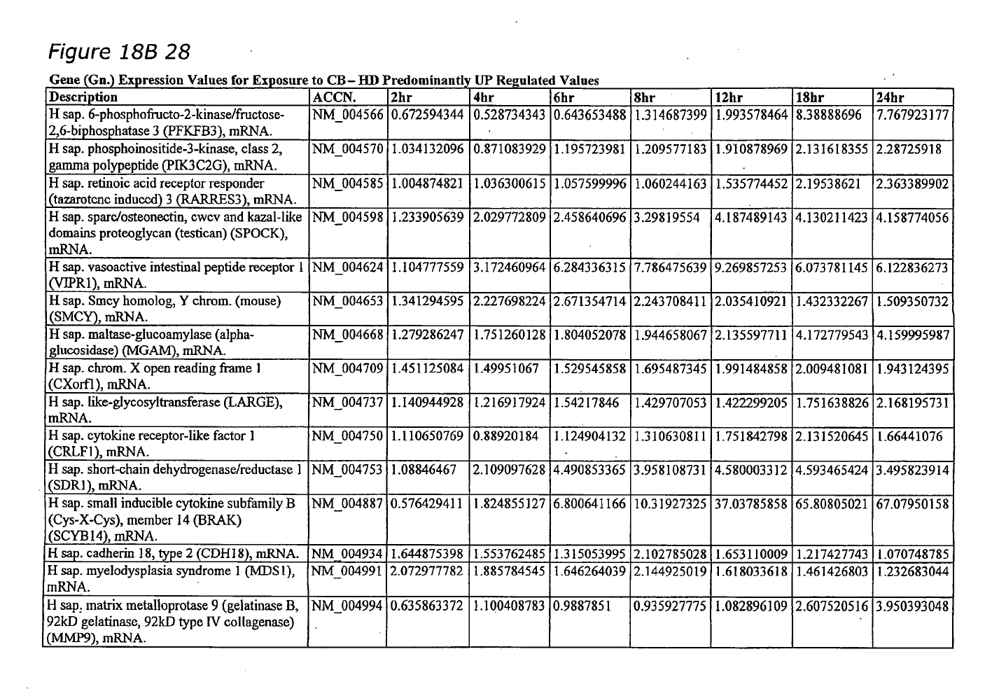

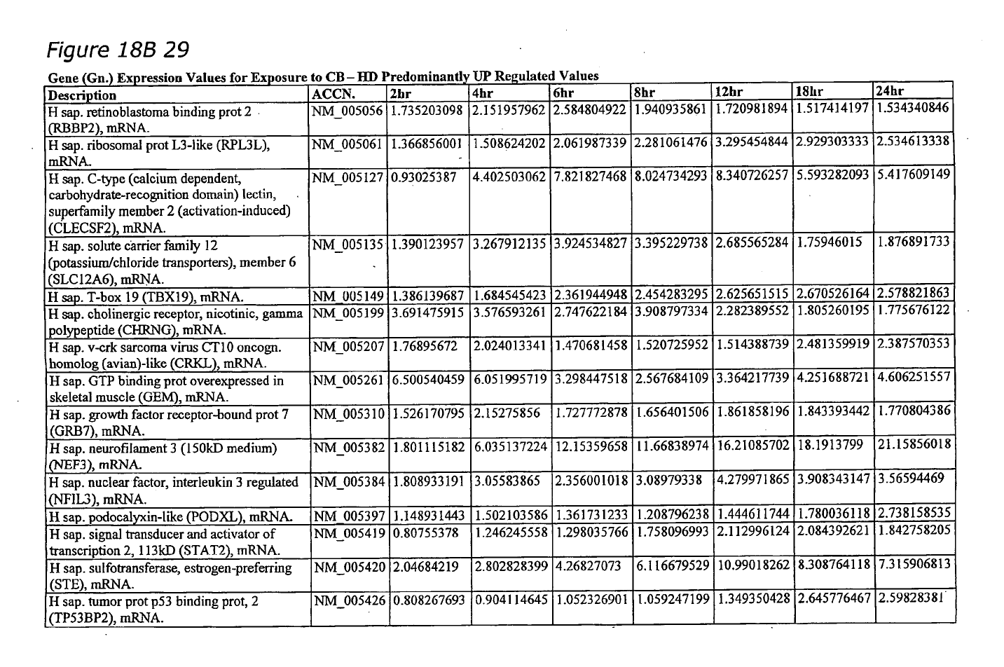

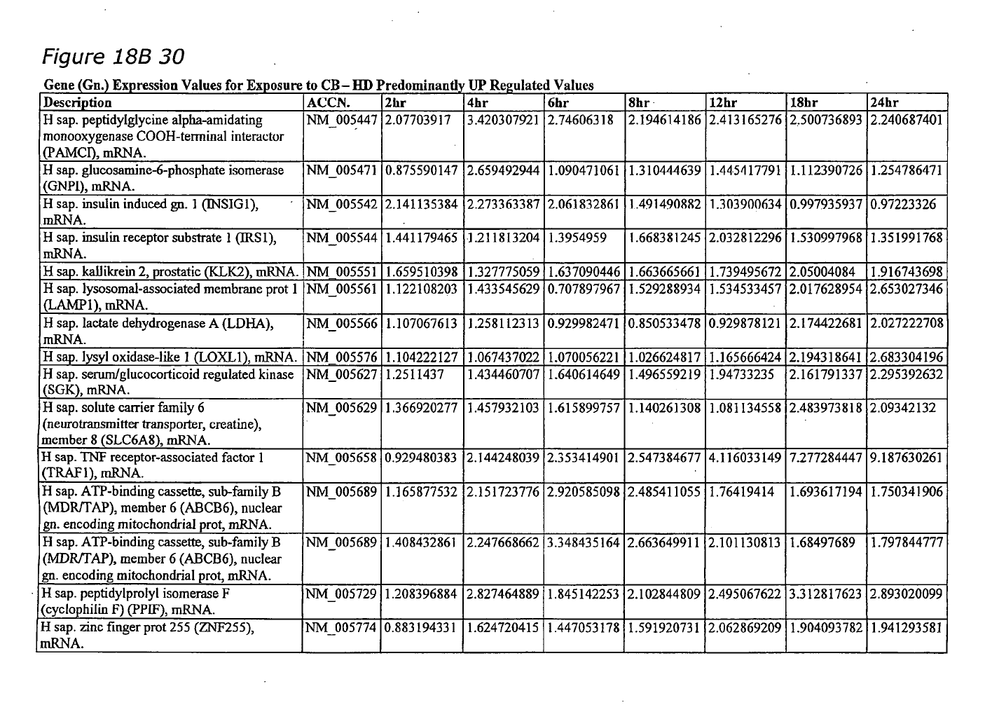

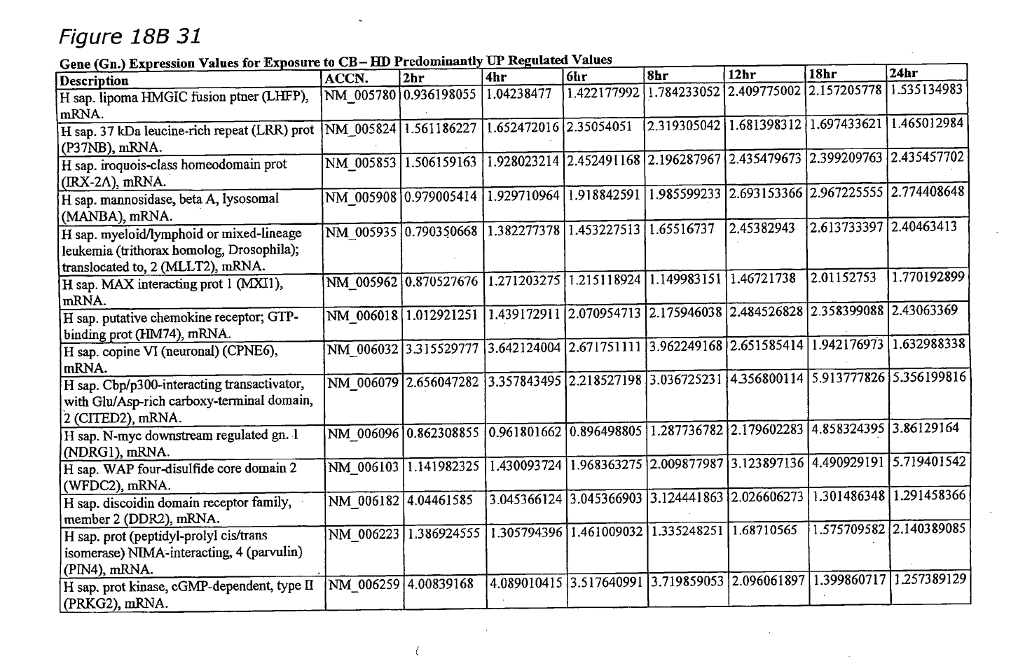

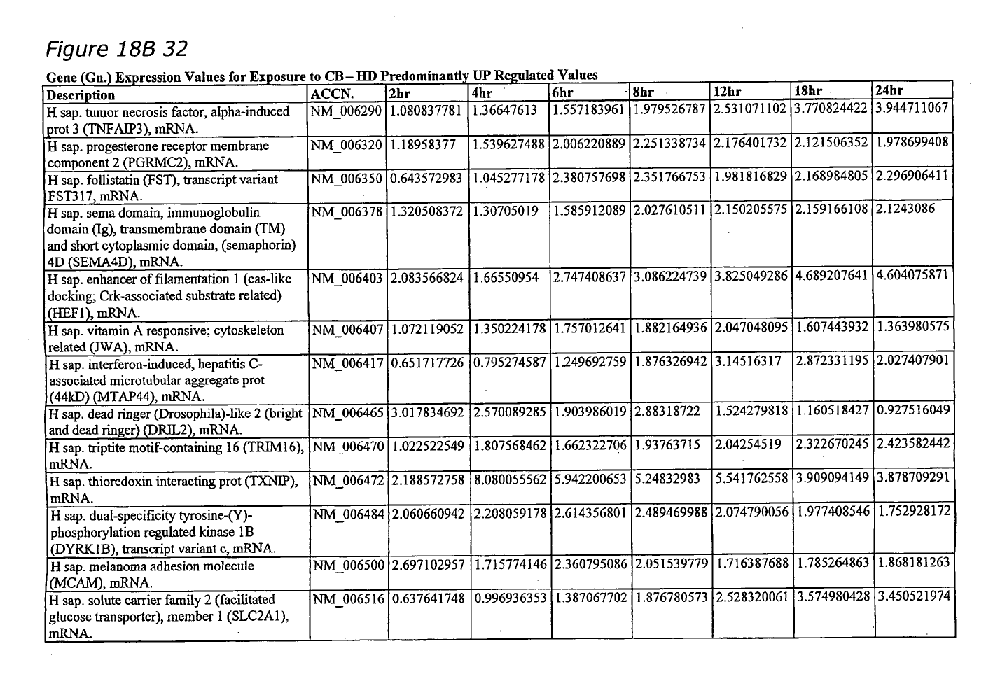

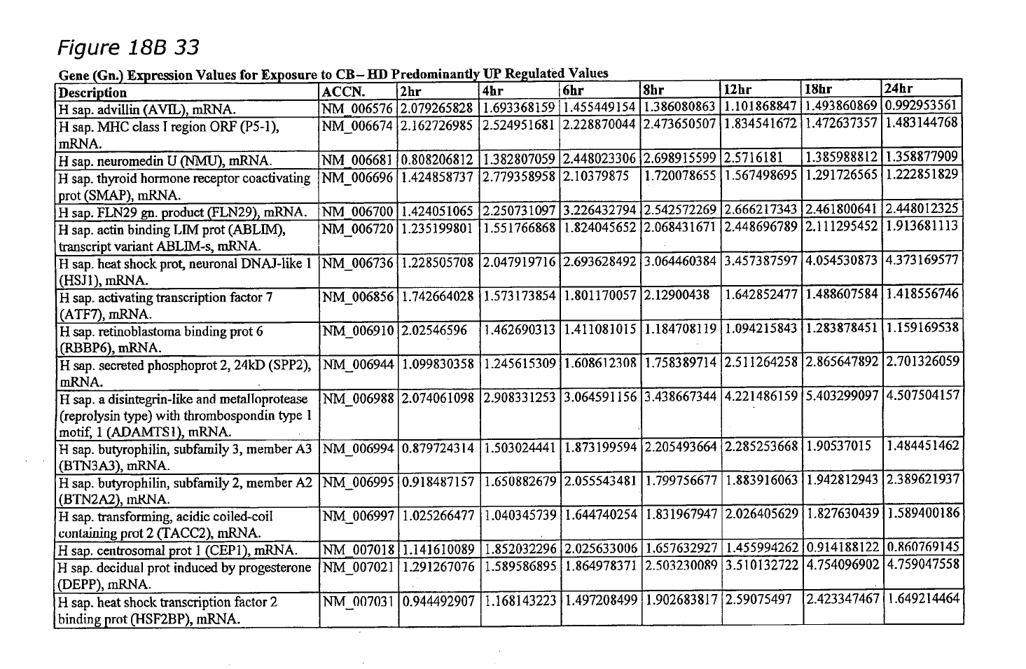

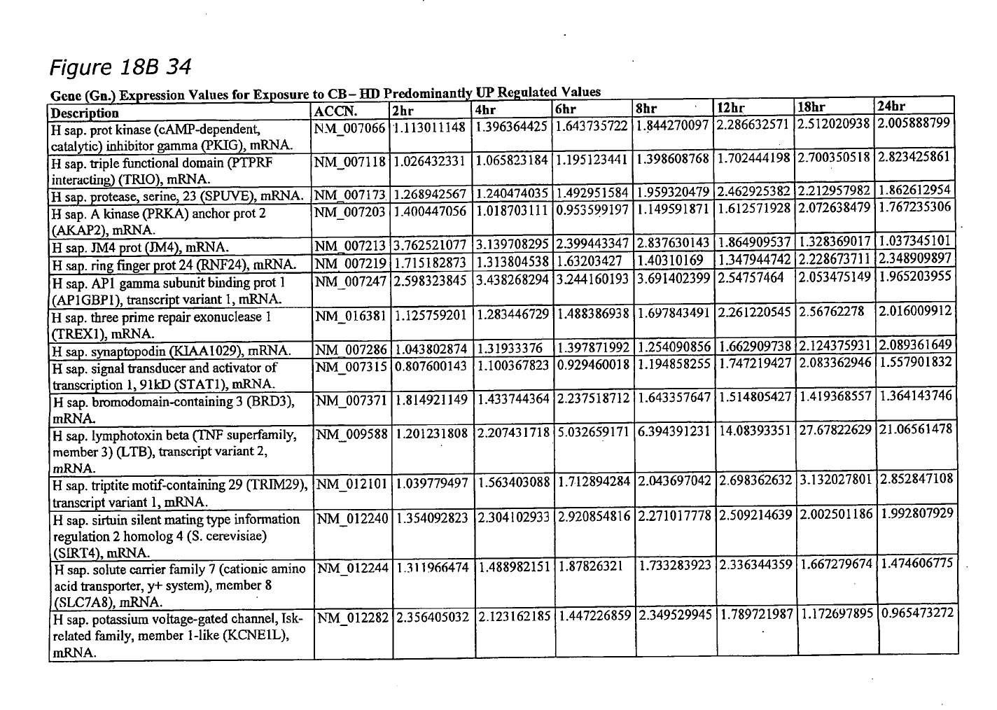

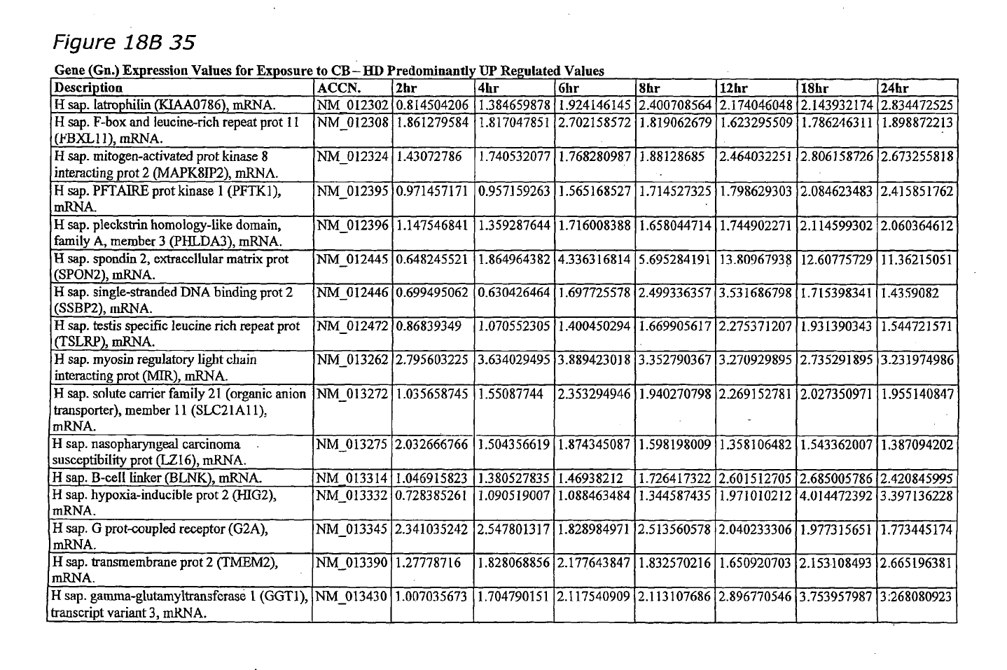

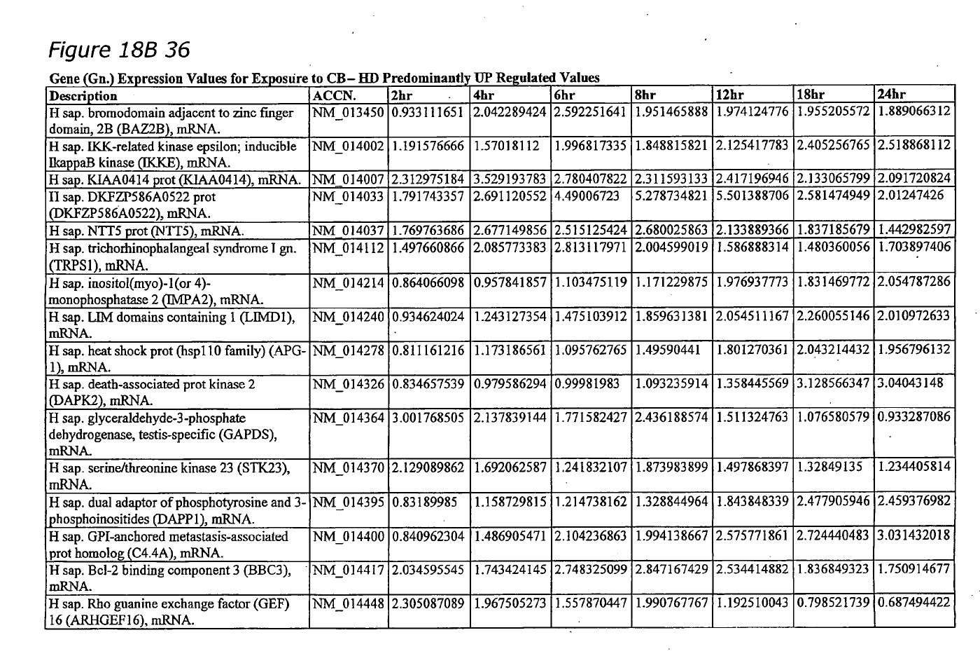

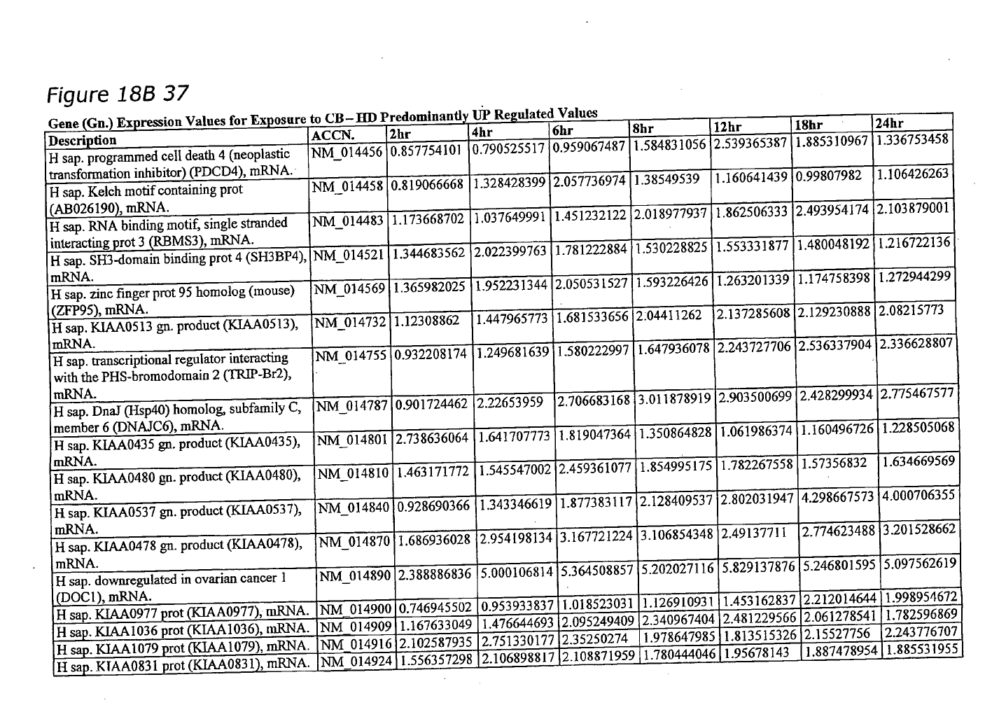

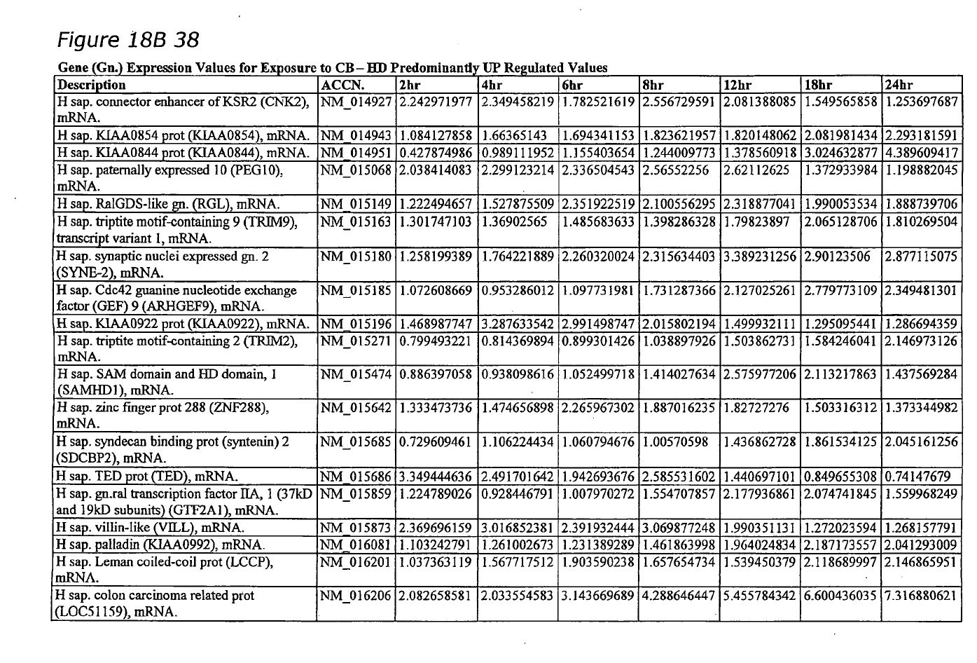

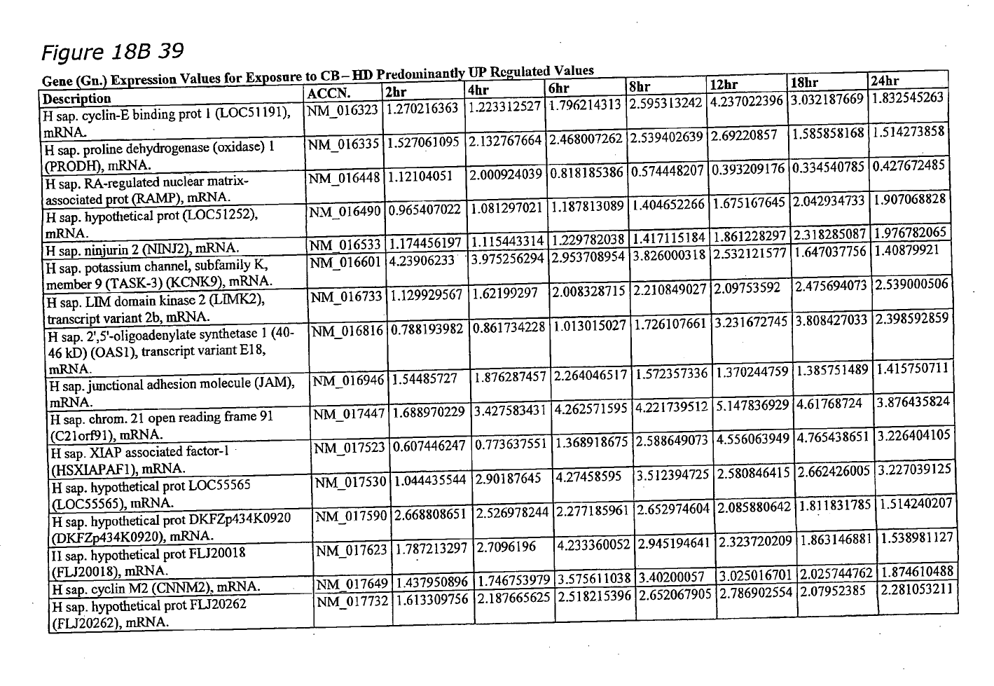

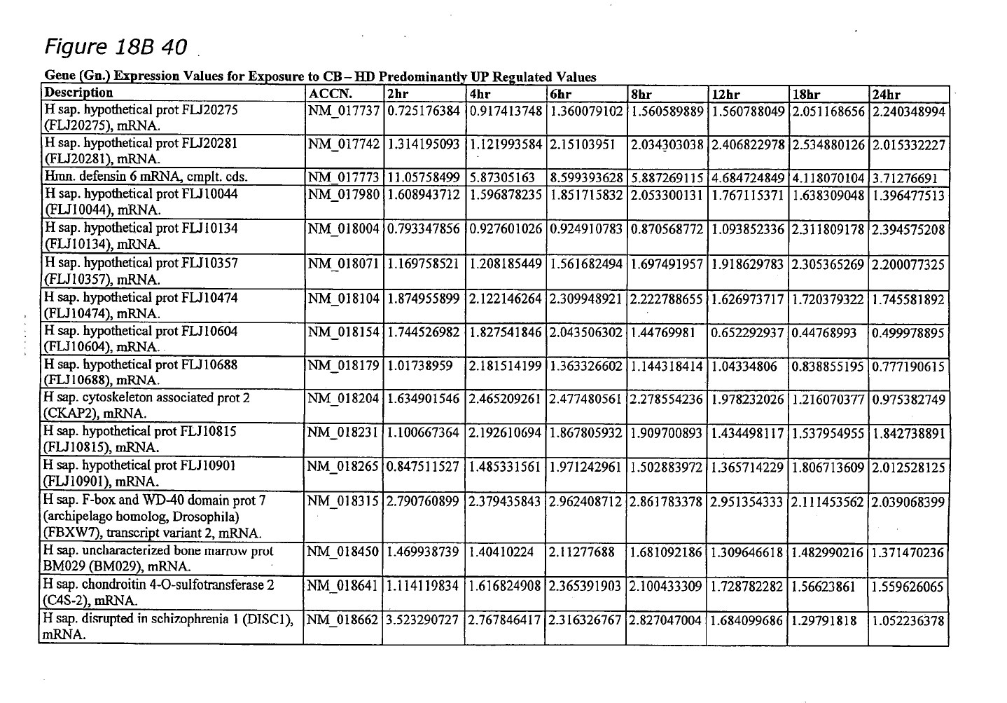

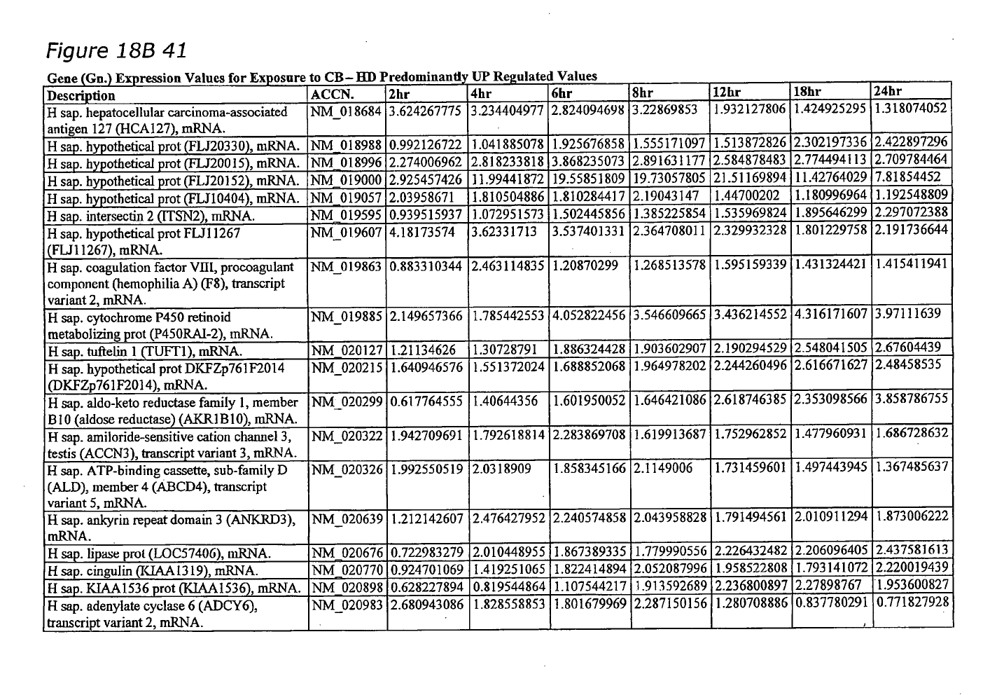

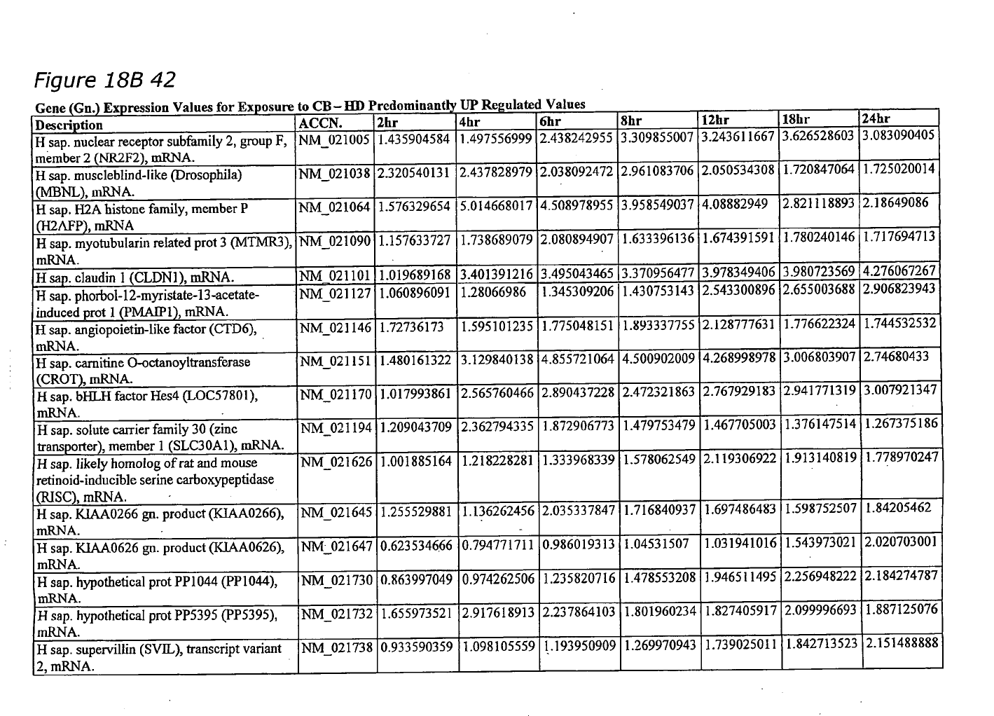

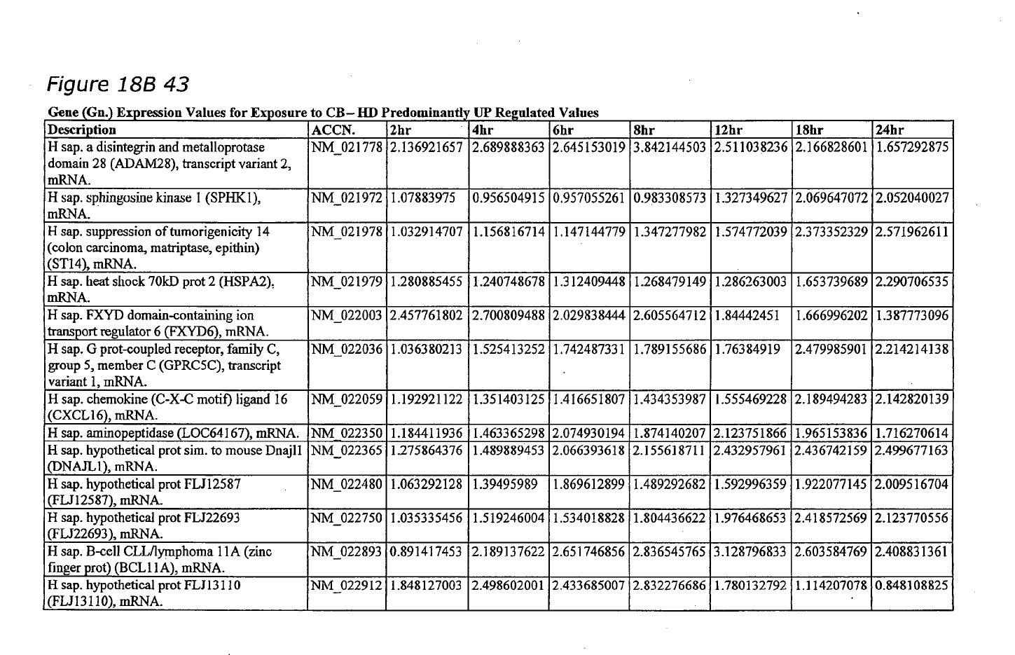

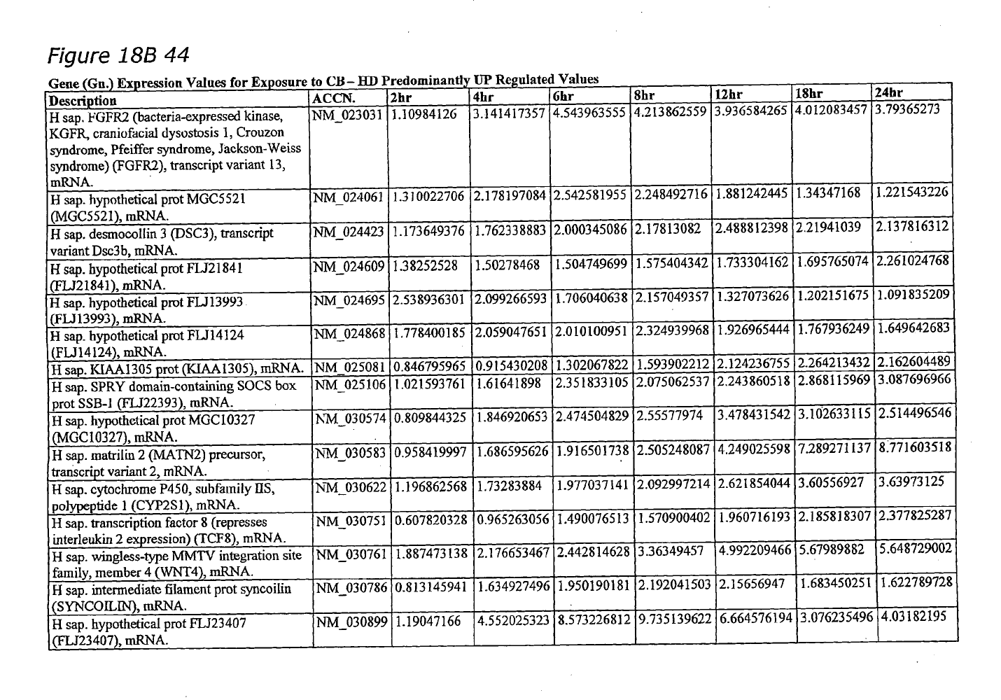

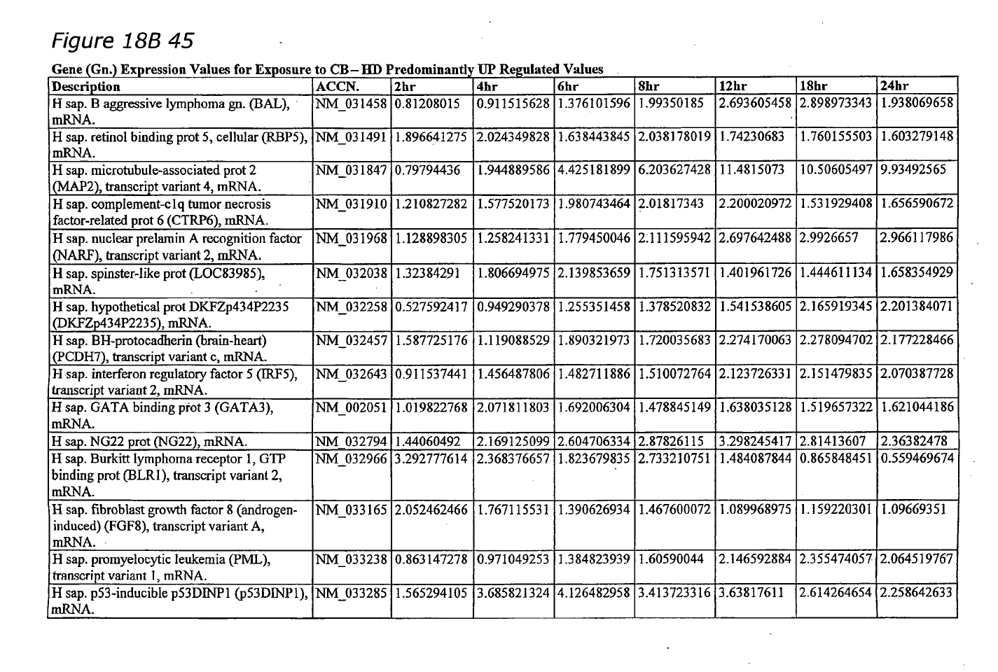

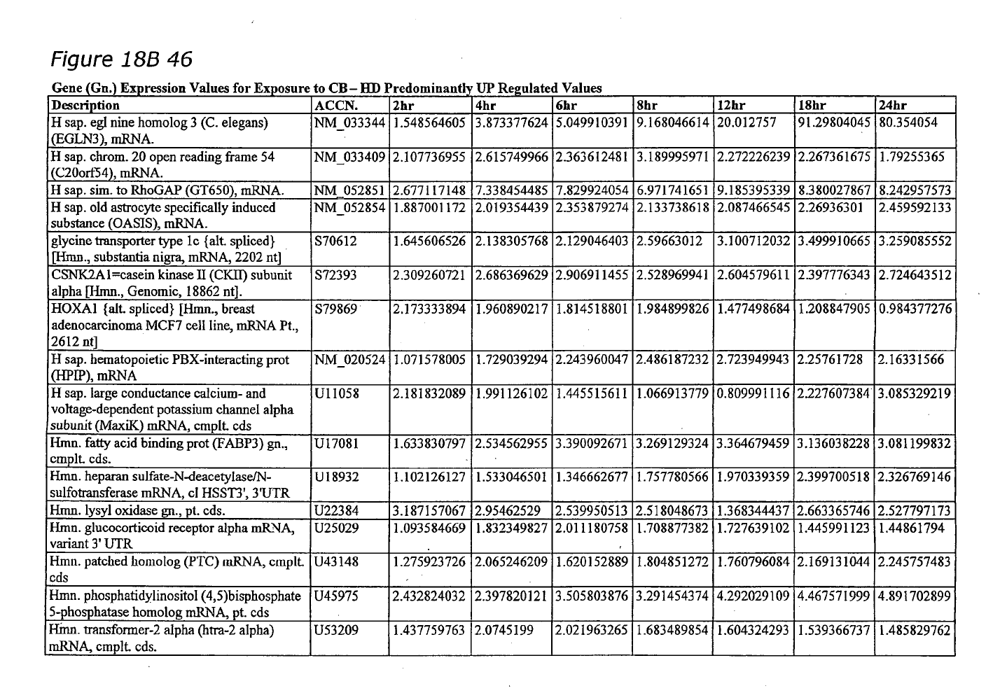

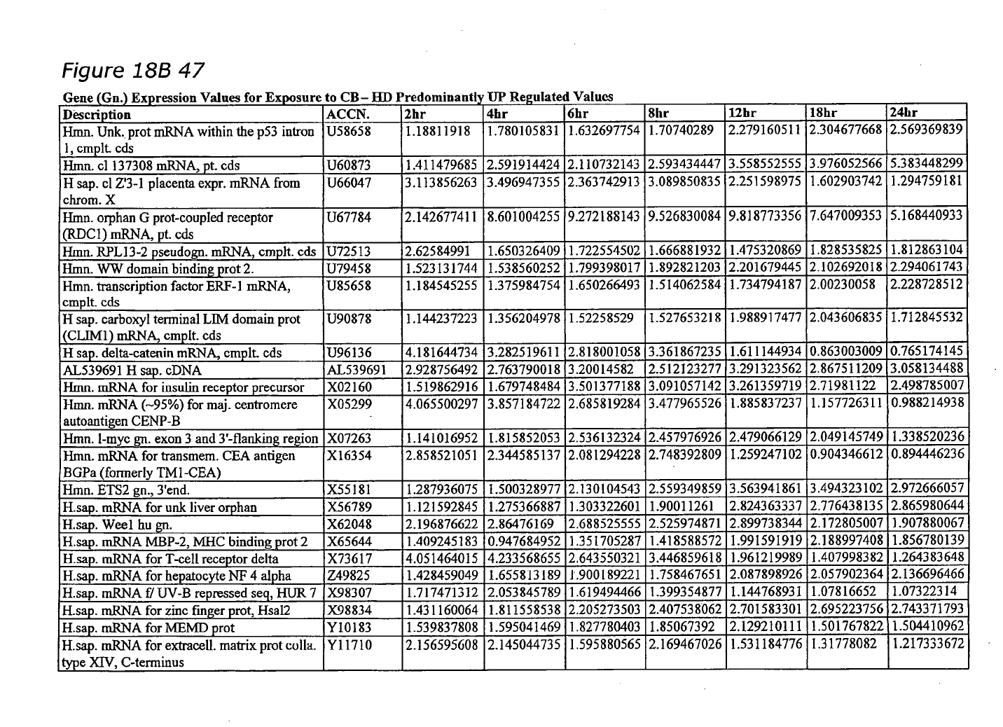

FIG. 18A1-74 presents GEM results for genes predominantly down-regulated in response to high dose CB nanoparticle exposure over time in HEK cells.

FIG. 18B1-47 presents GEM results for genes predominantly up-regulated in response to high dose CB nanoparticle exposure over time in HEK cells.

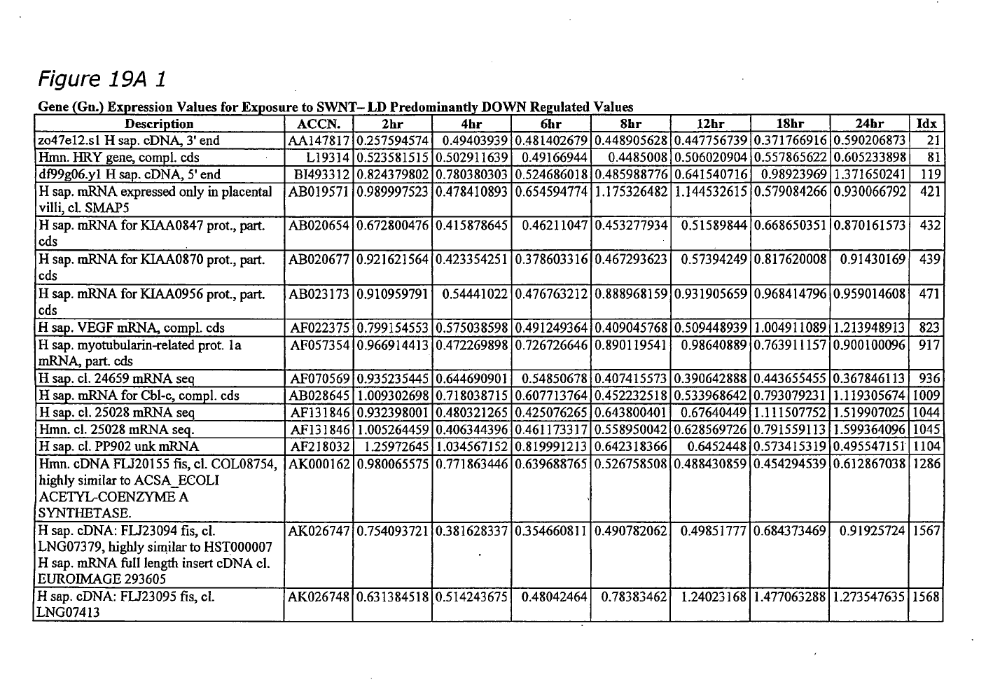

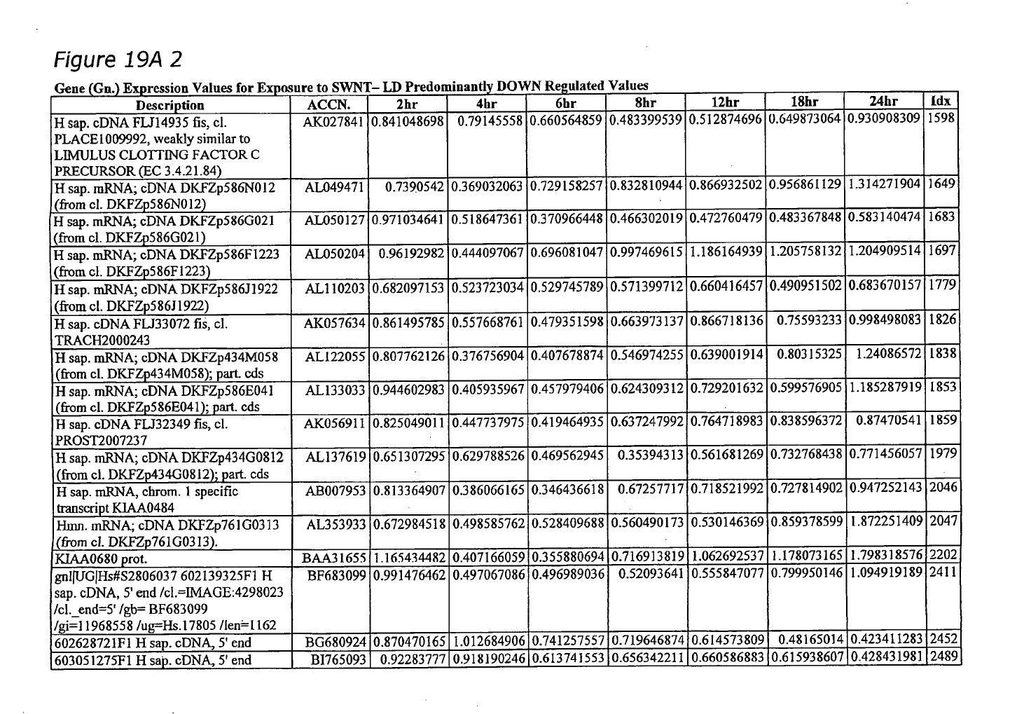

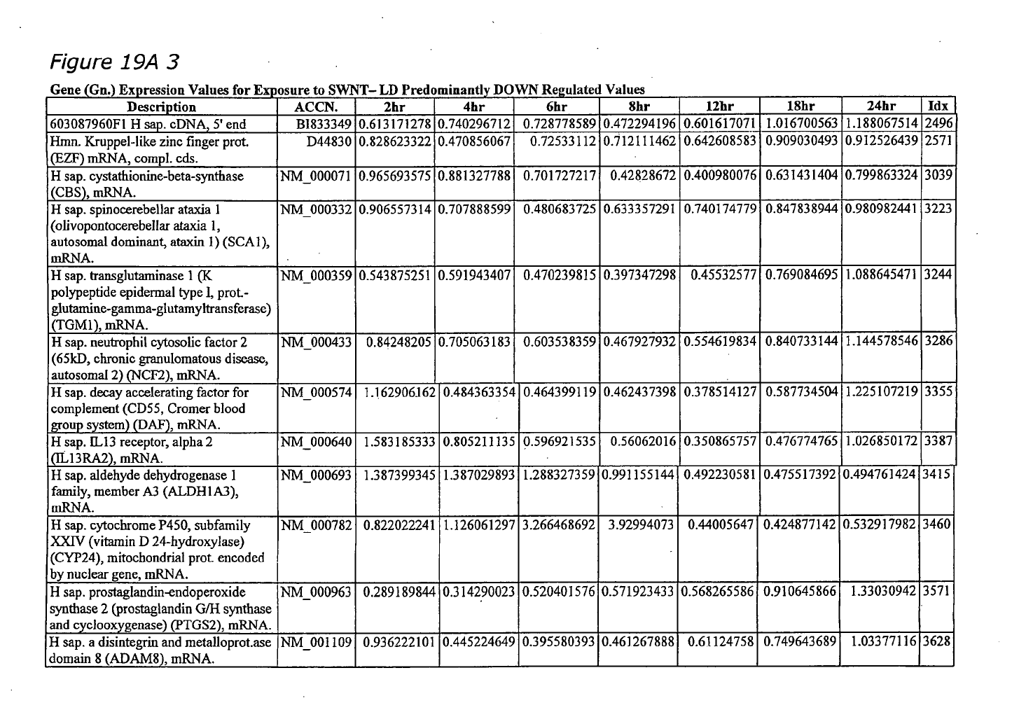

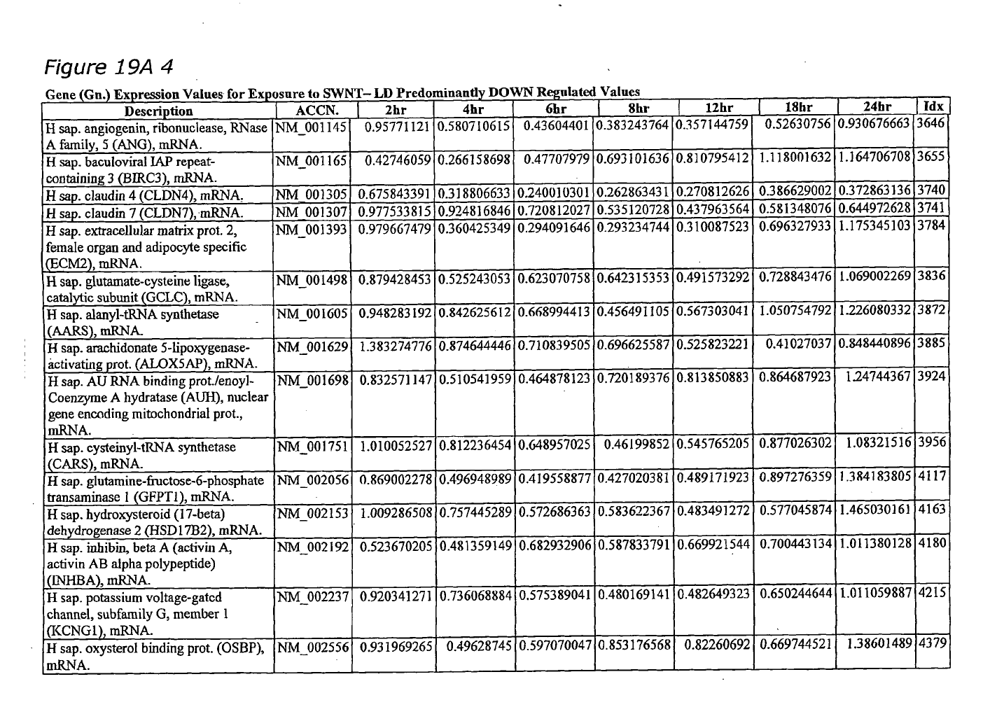

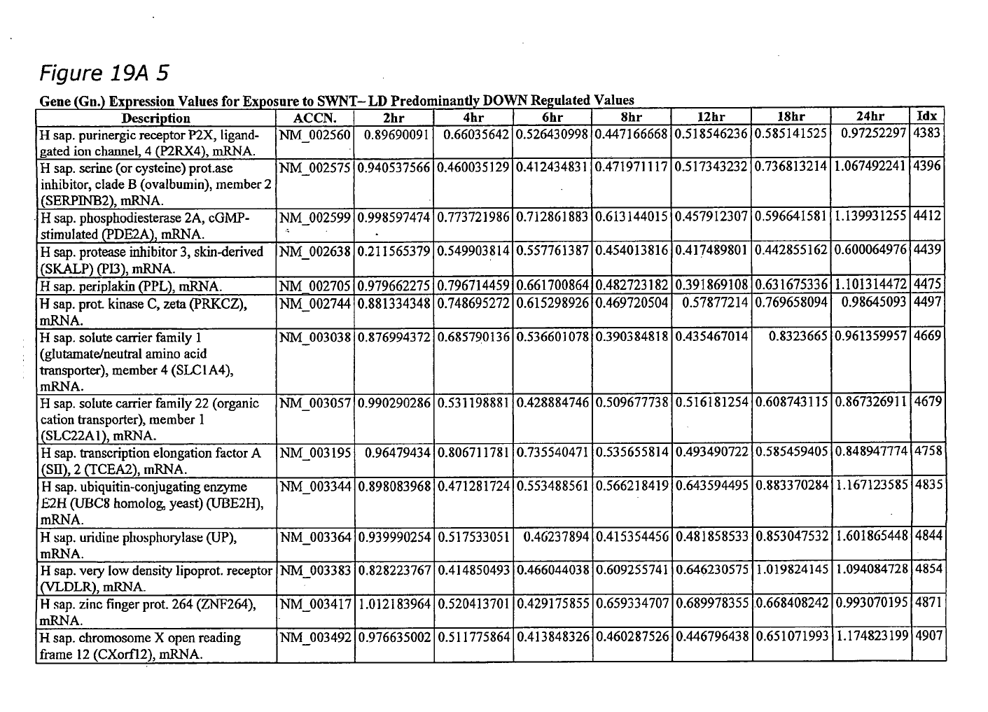

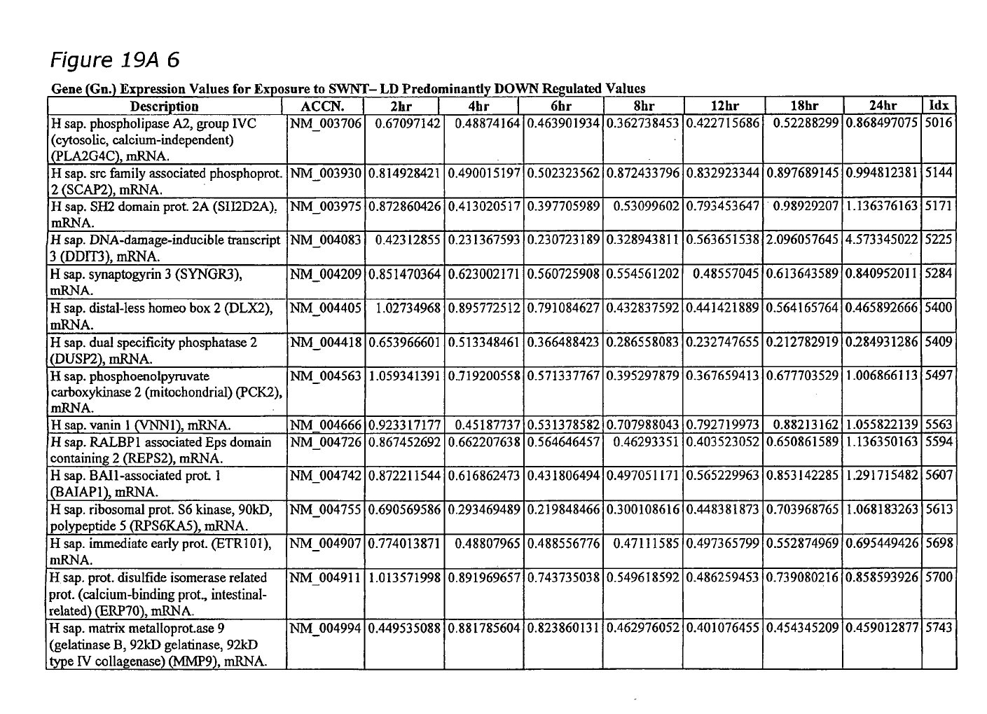

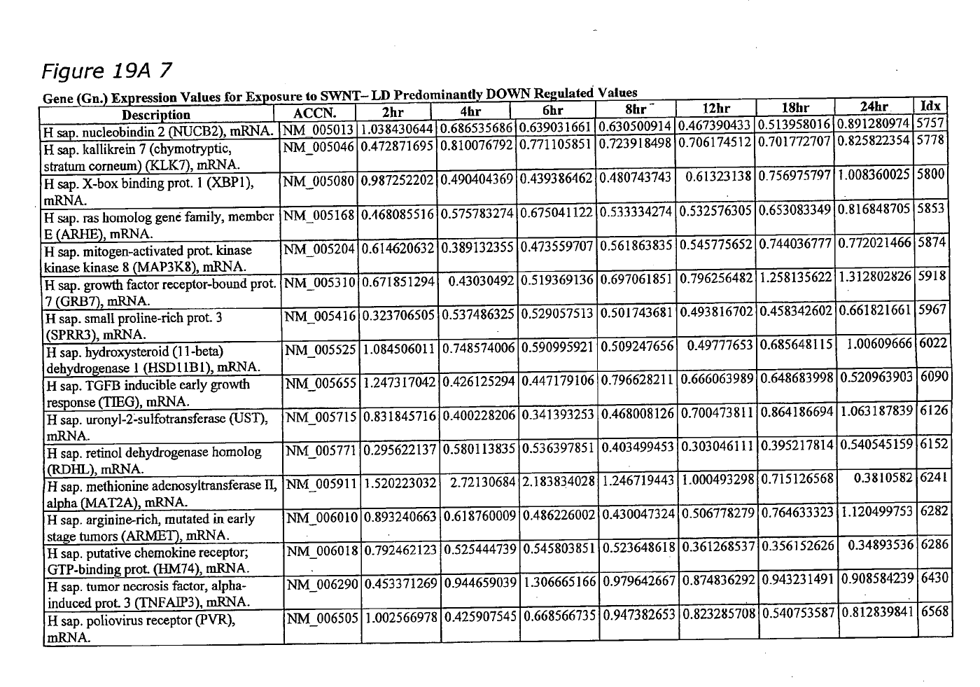

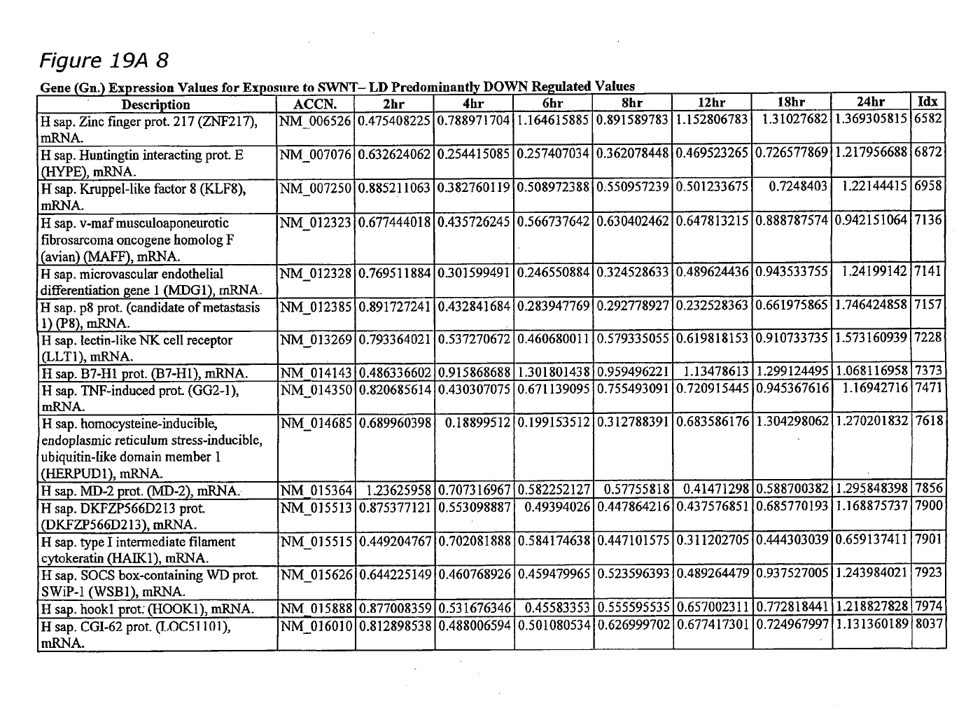

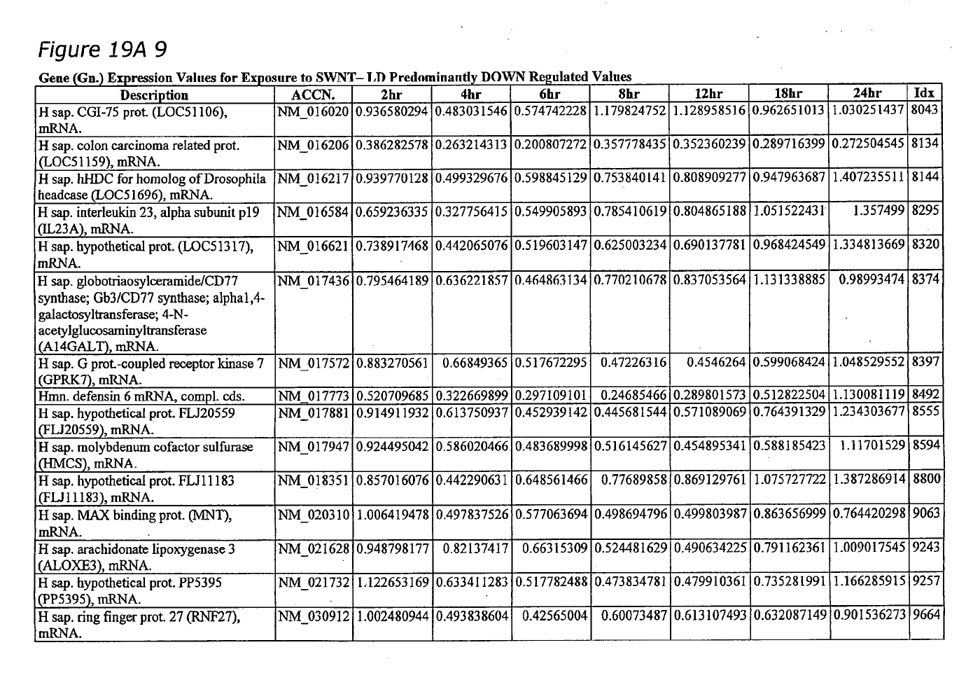

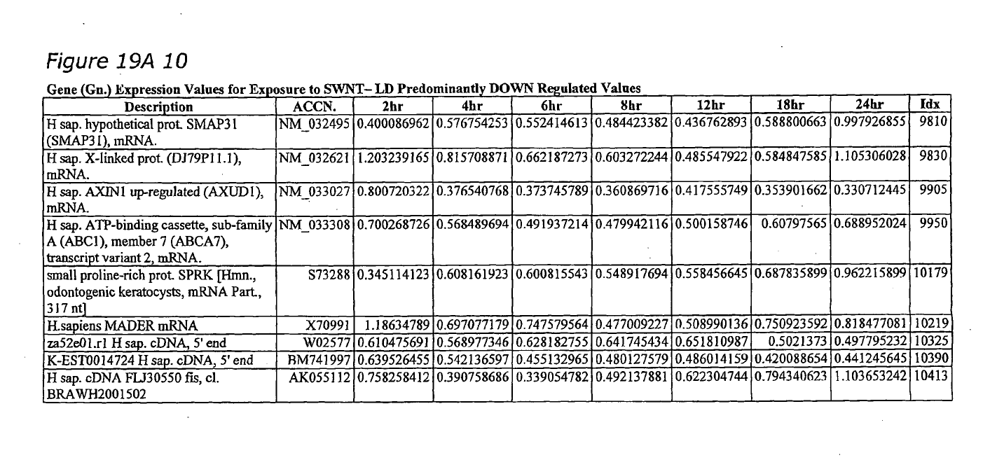

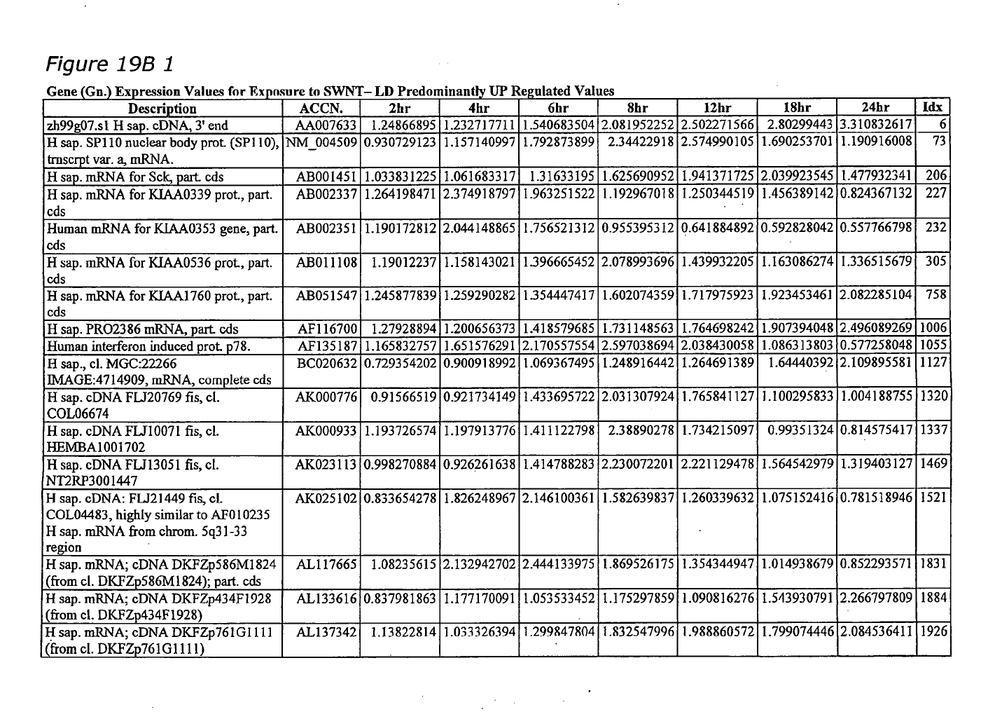

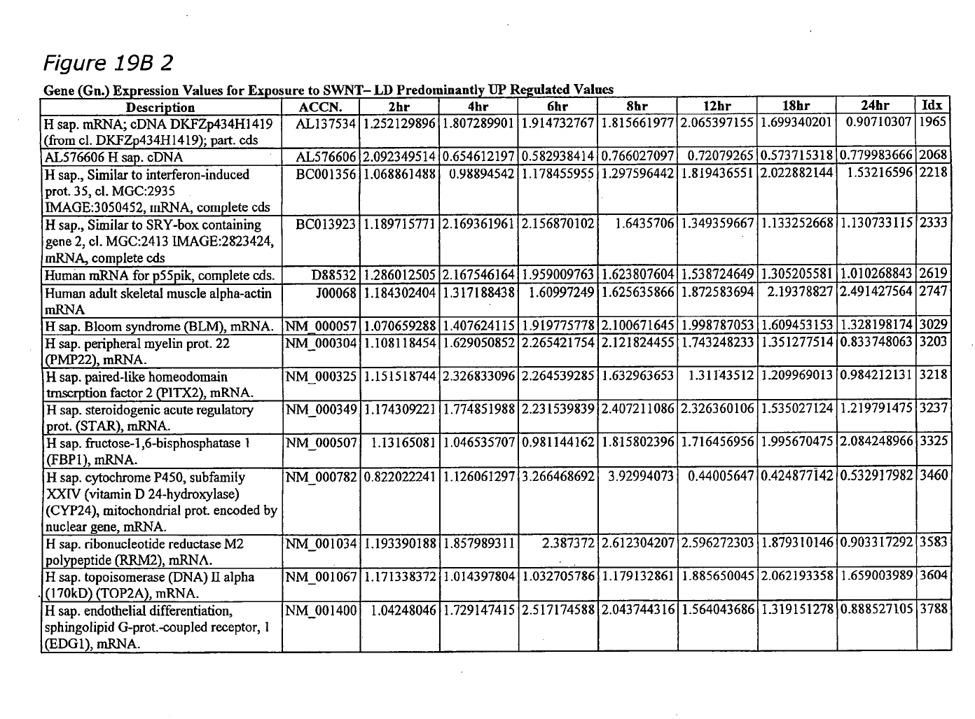

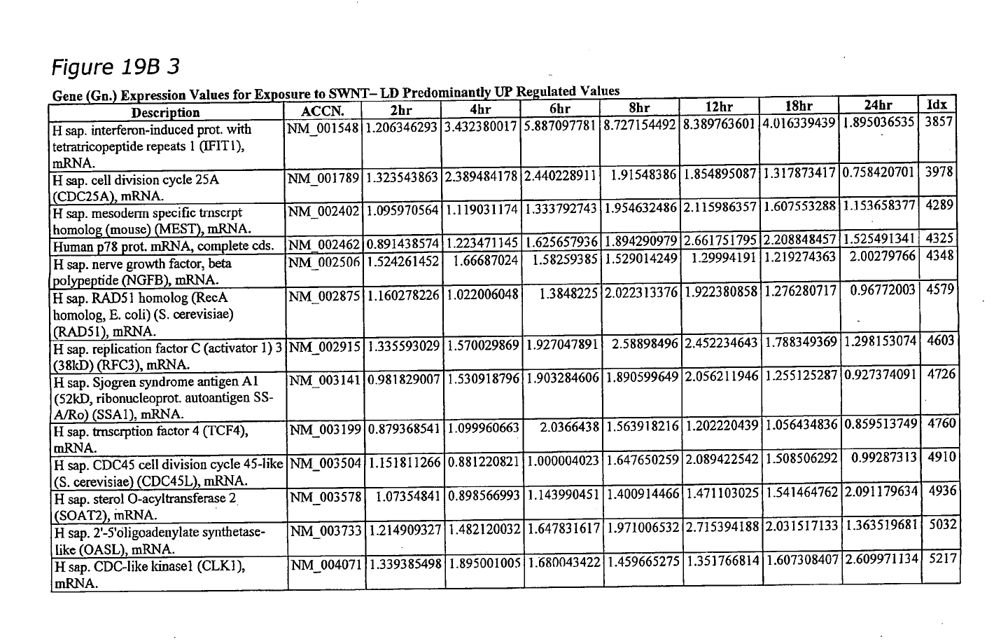

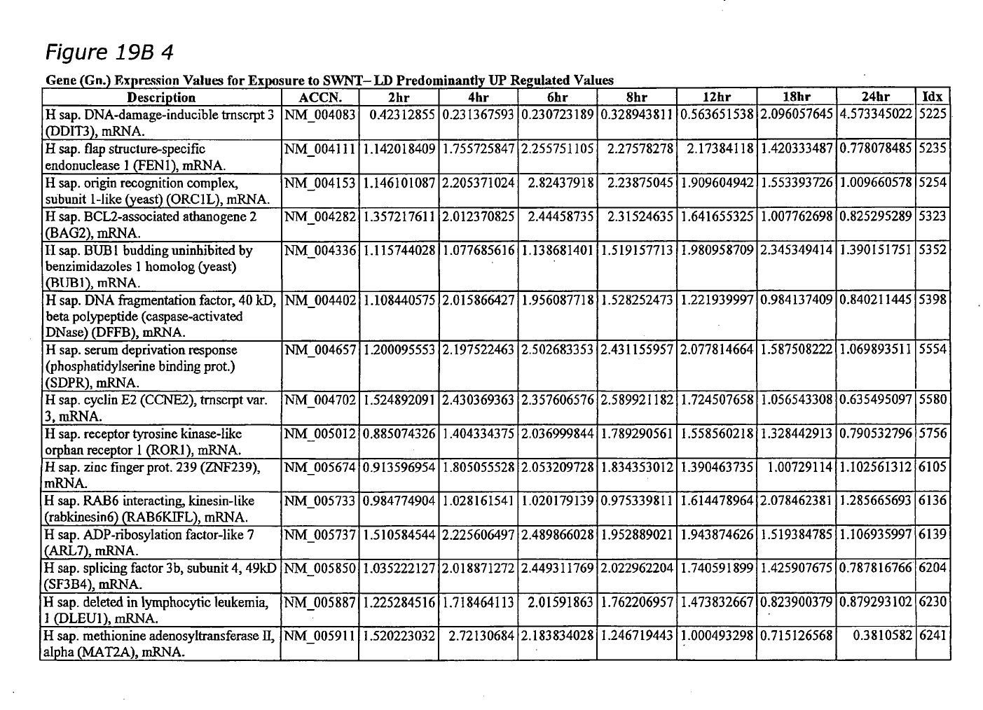

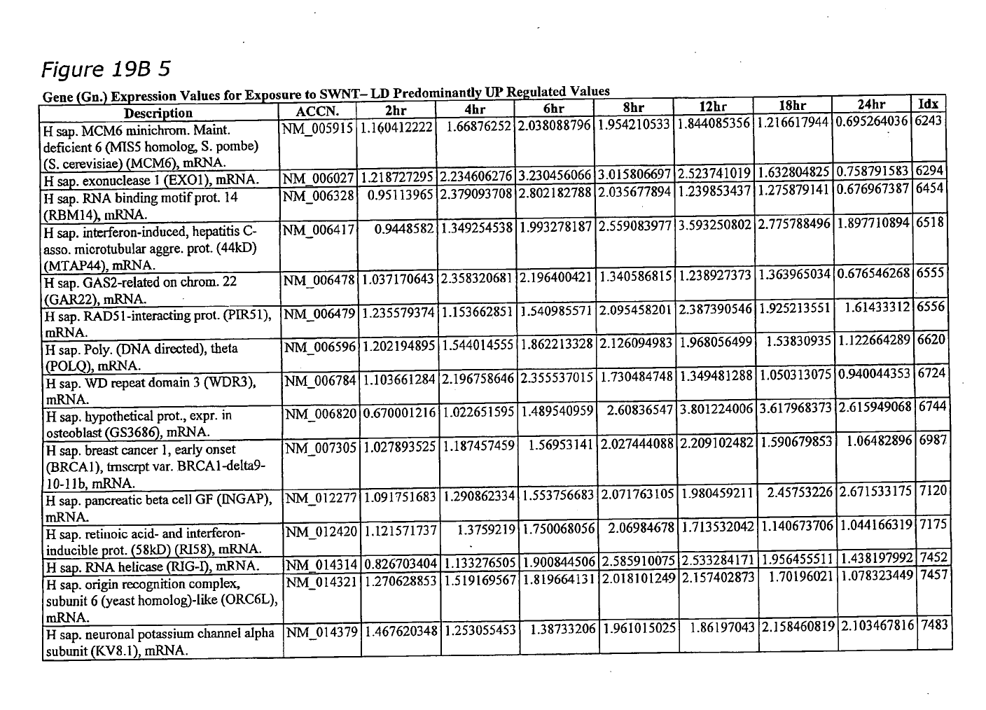

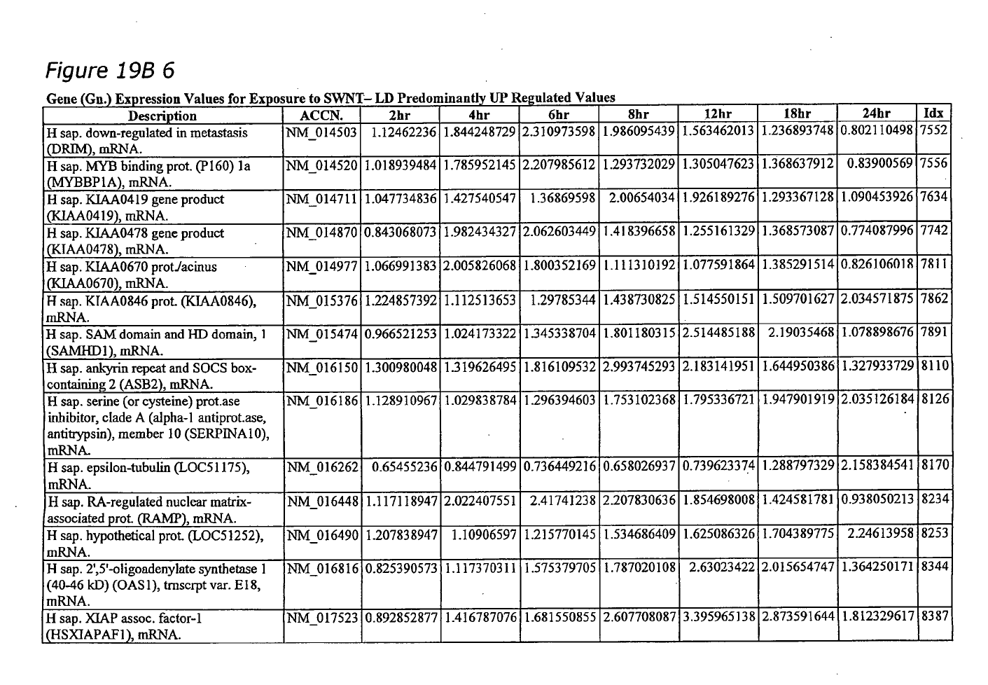

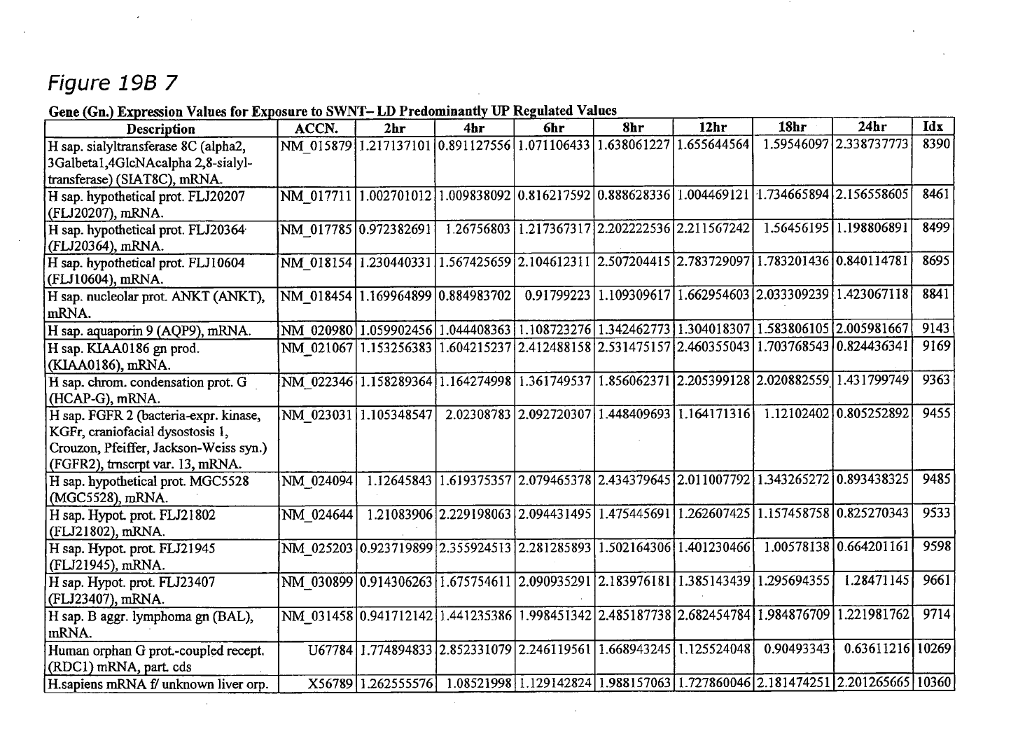

FIG. 19A1-10 presents GEM results for genes predominantly down-regulated in response to low dose SWNT nanoparticle exposure over time in HEK cells.

FIG. 19B1-7 presents GEM results for genes predominantly up-regulated in response to low dose SWNT nanoparticle exposure over time in HEK cells.

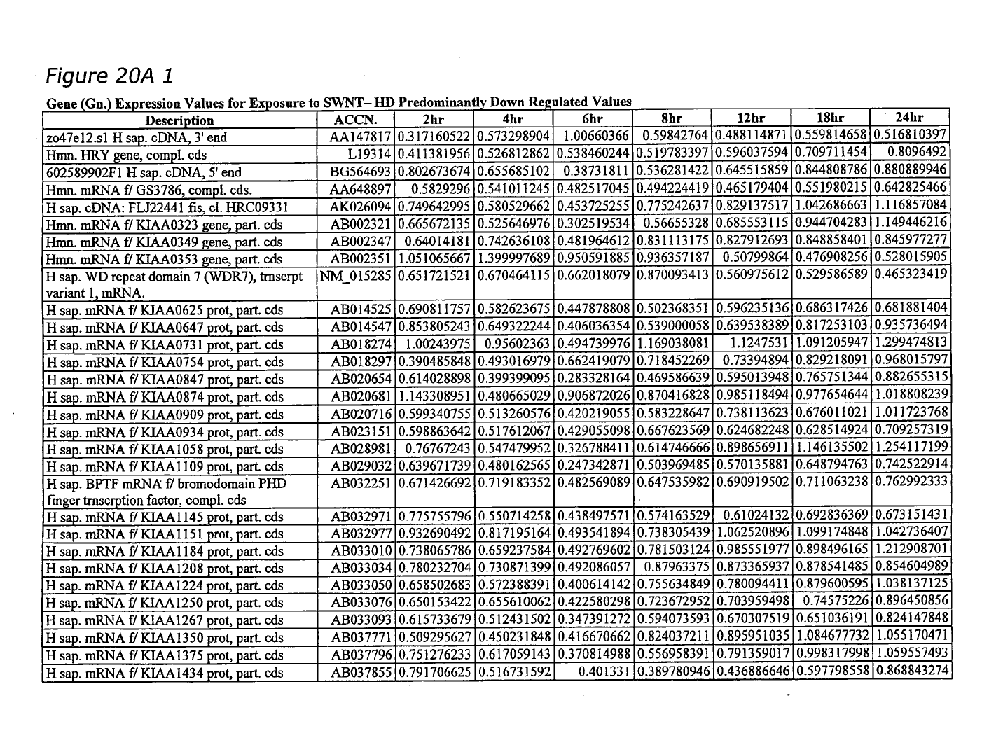

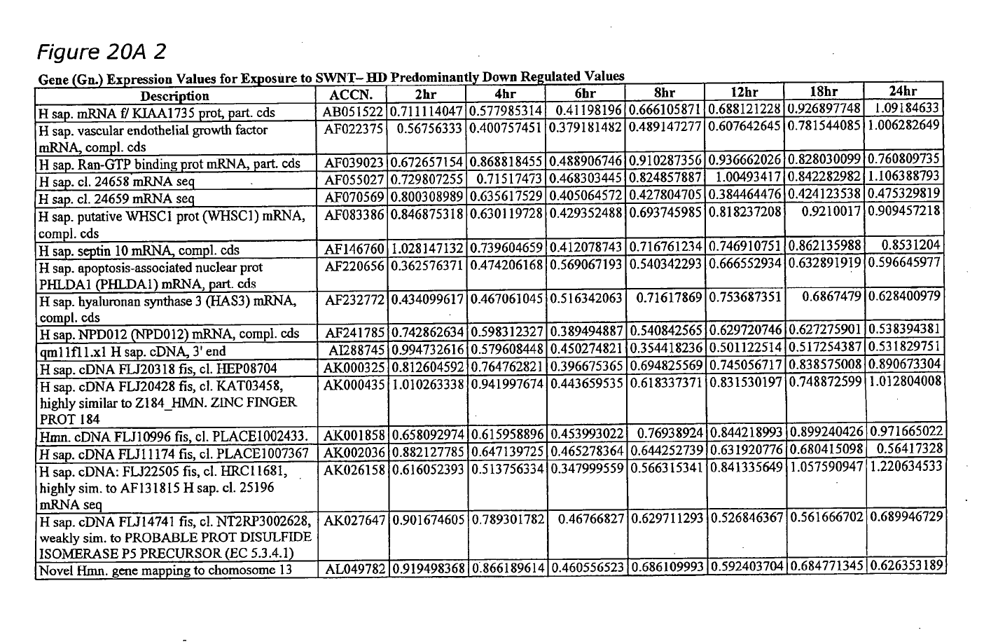

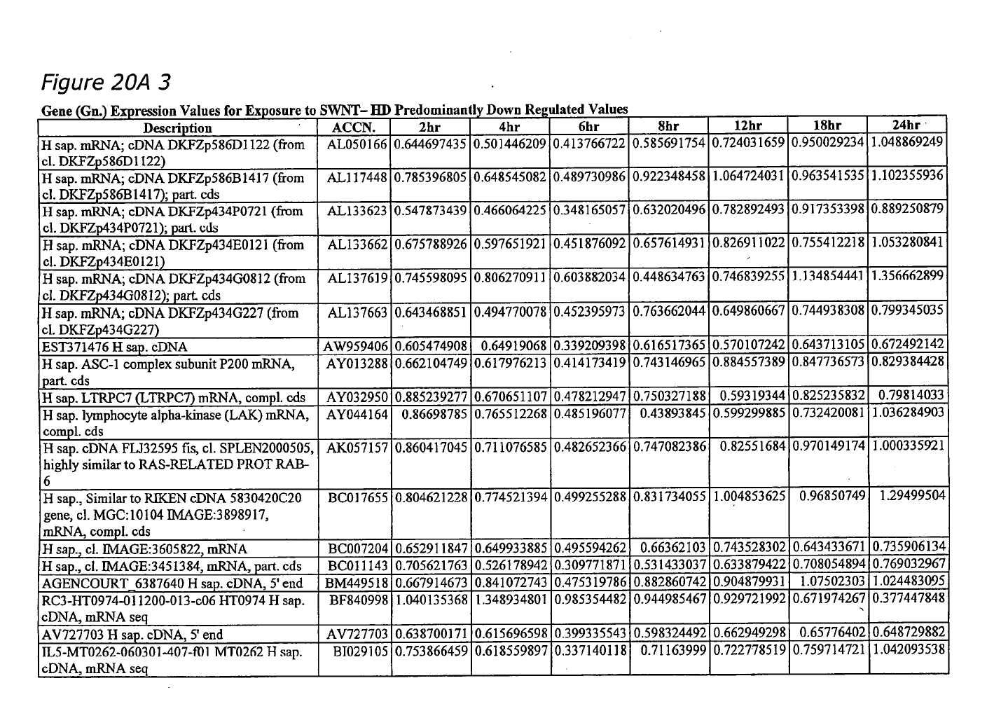

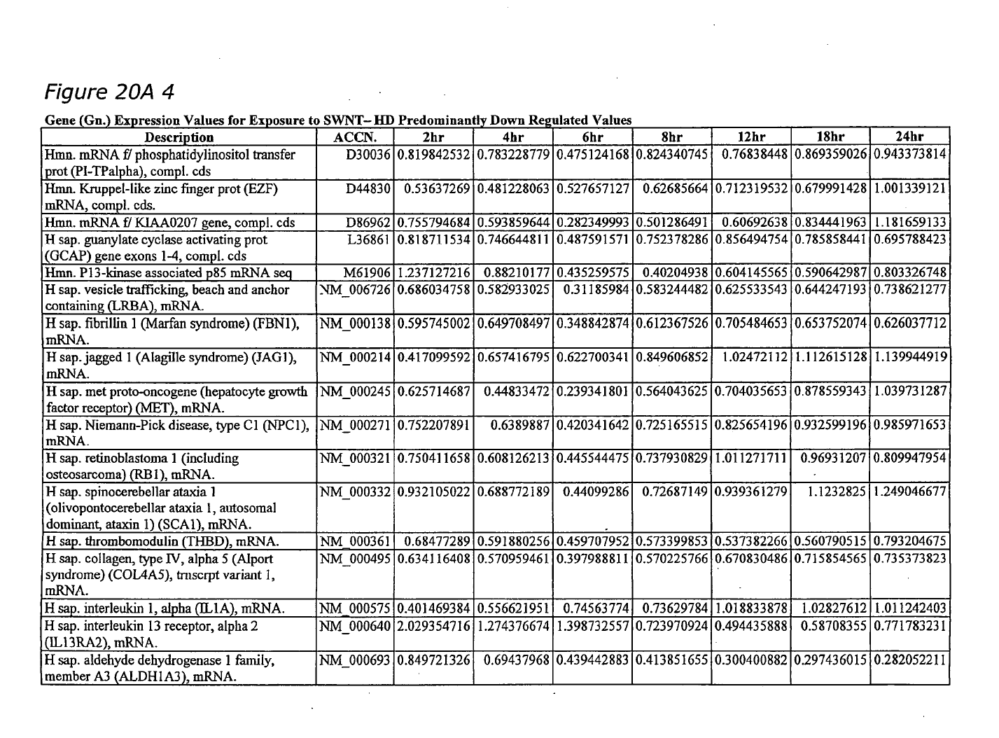

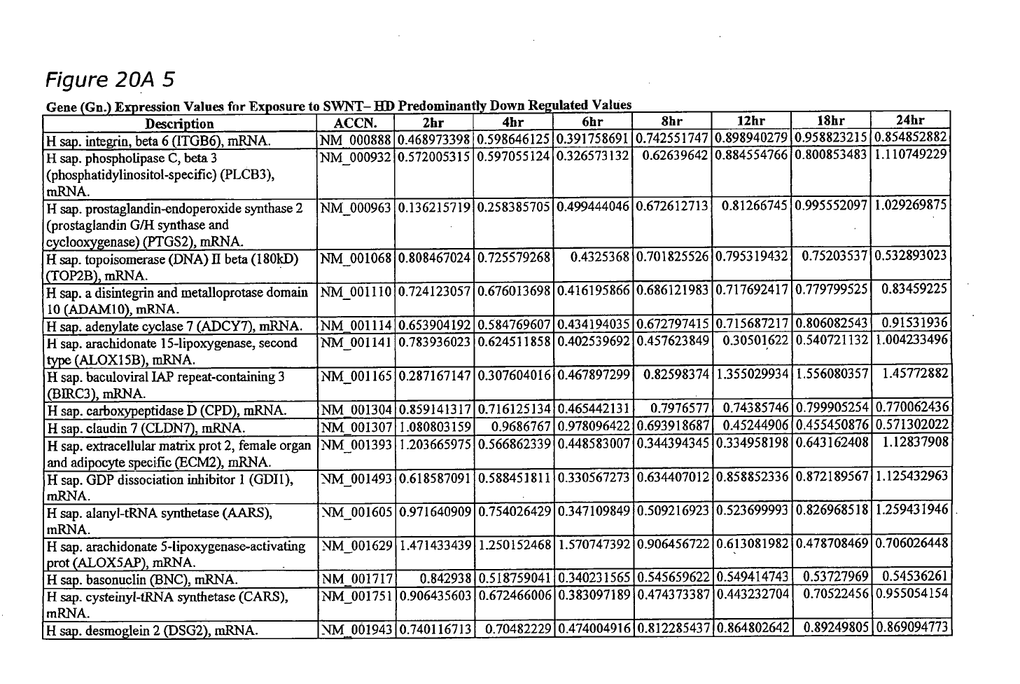

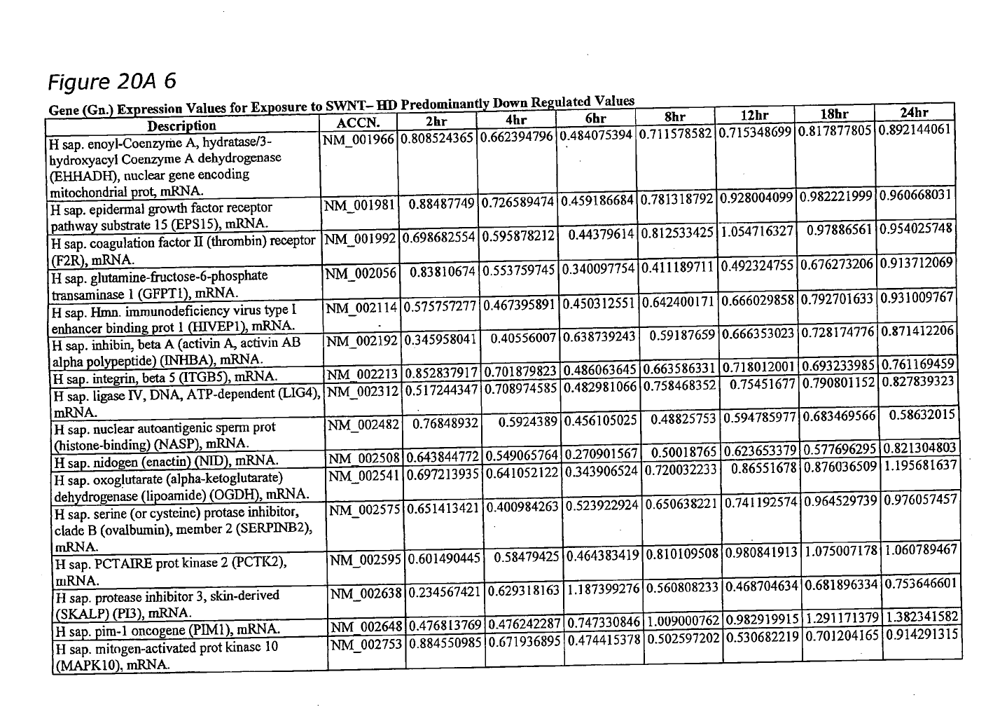

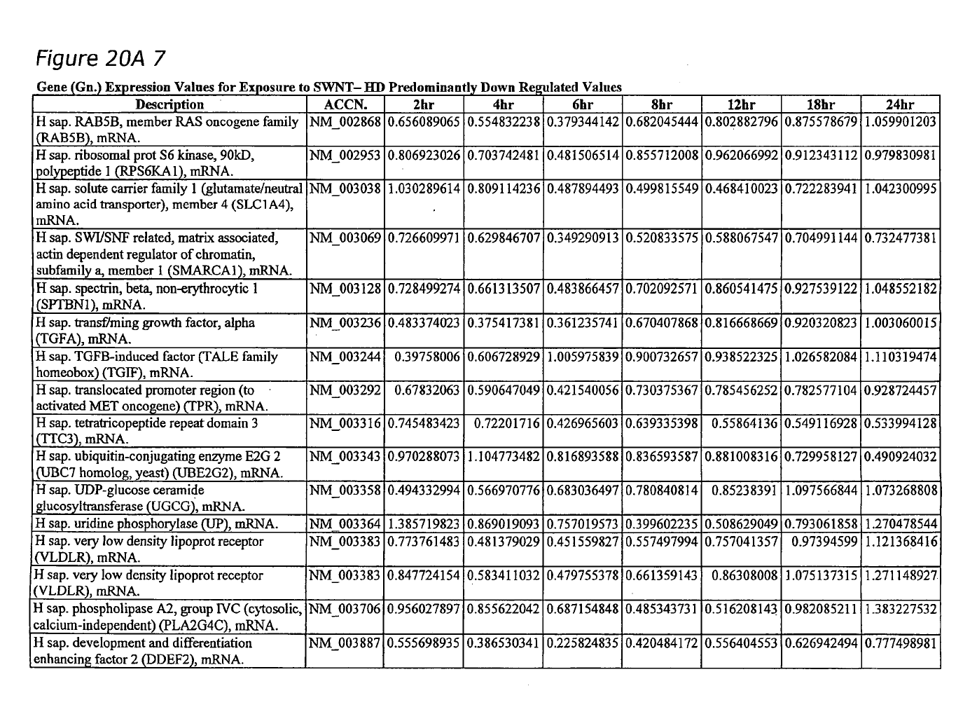

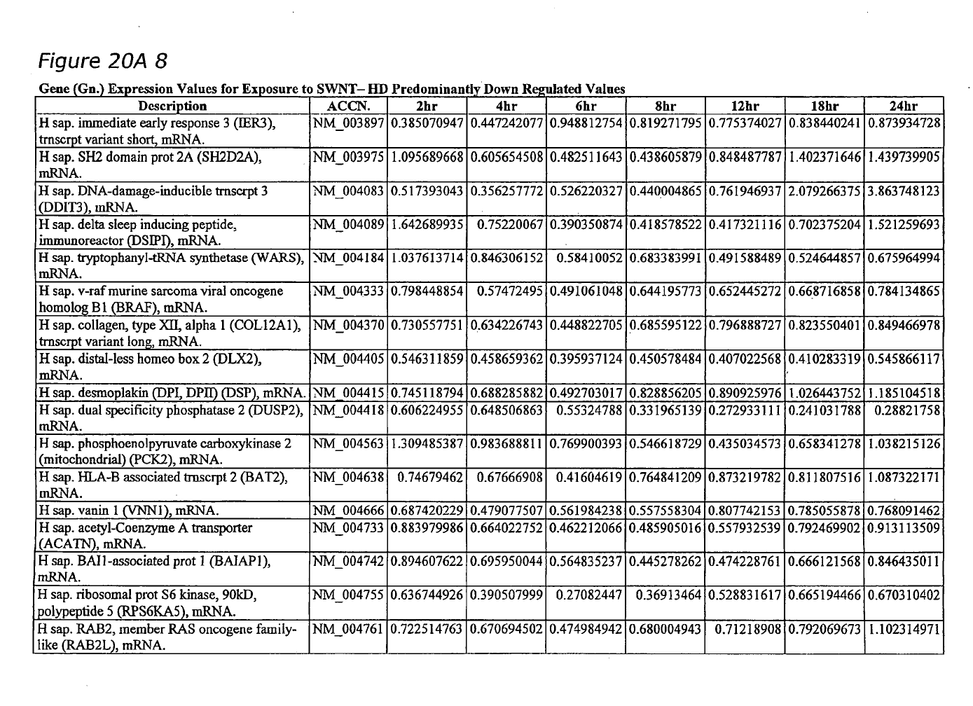

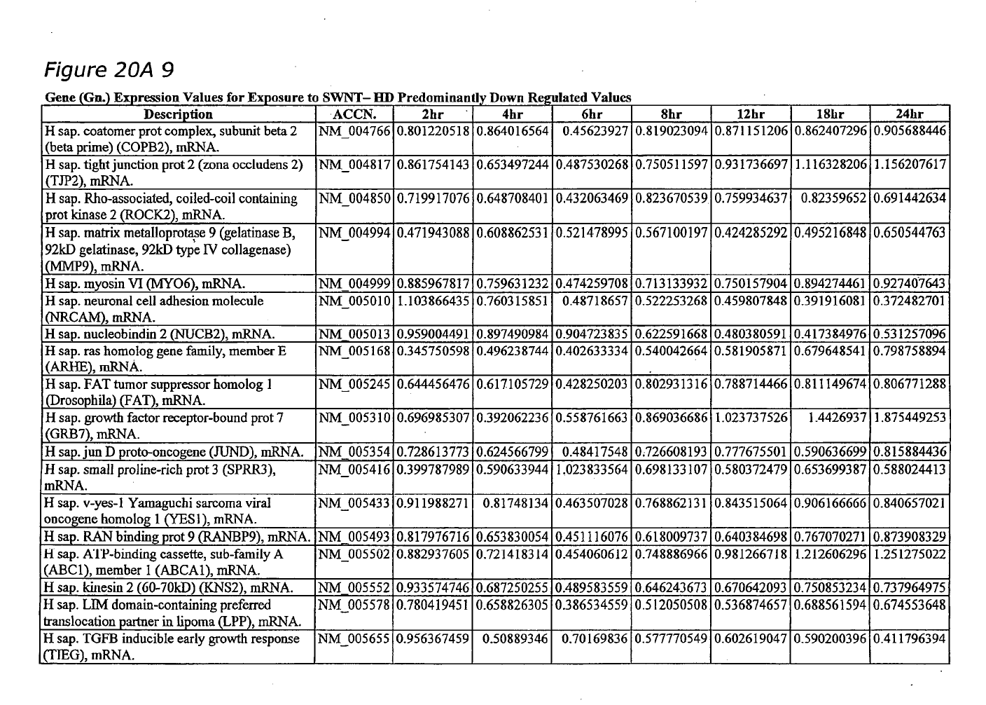

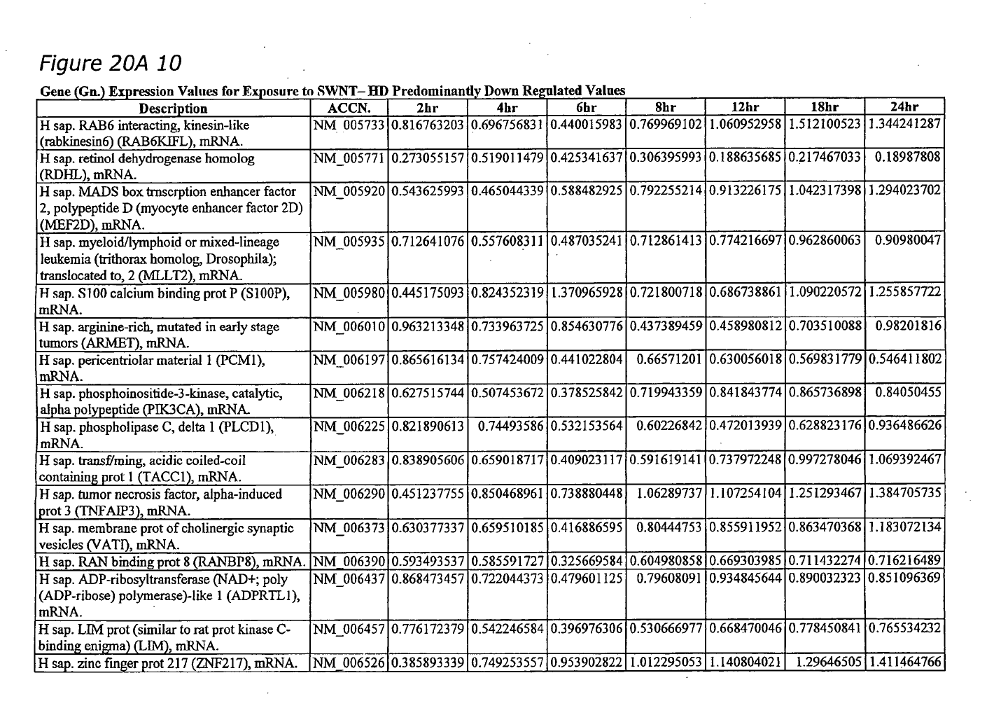

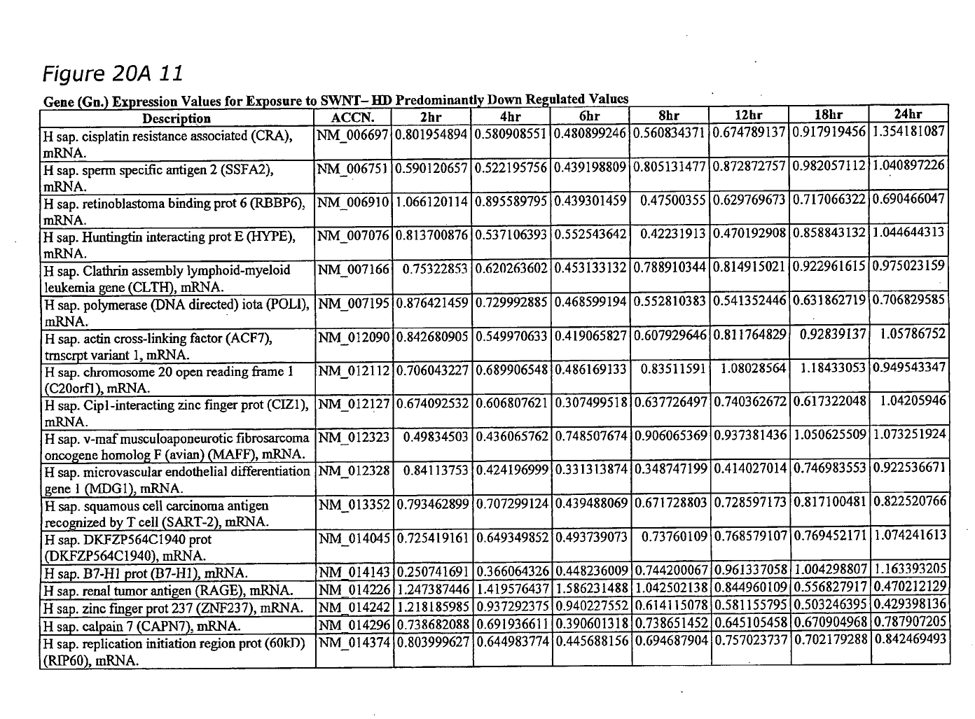

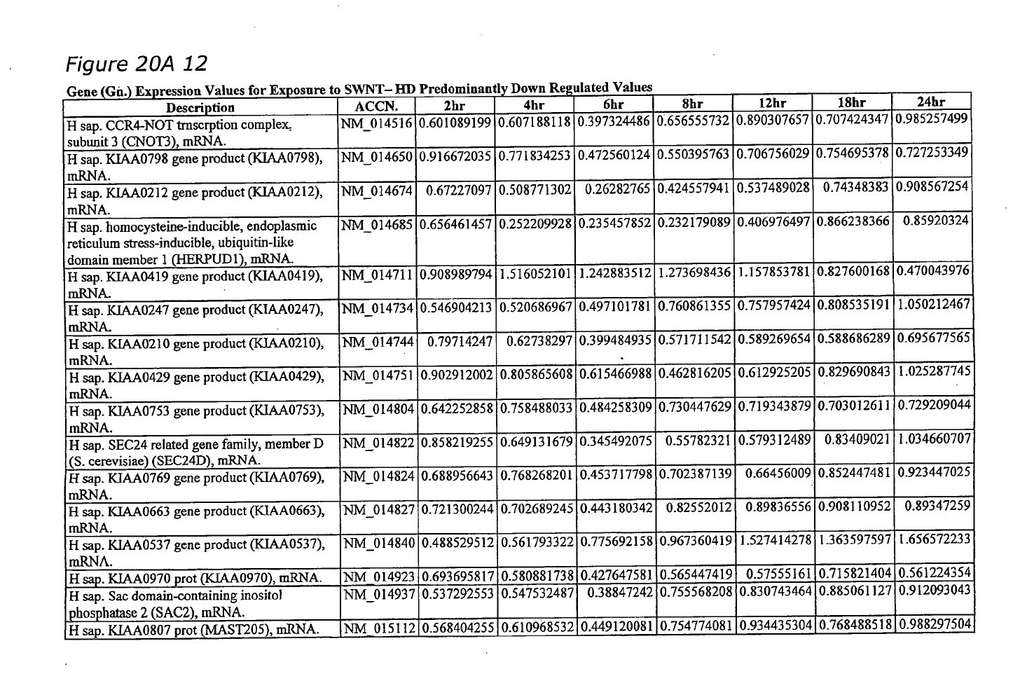

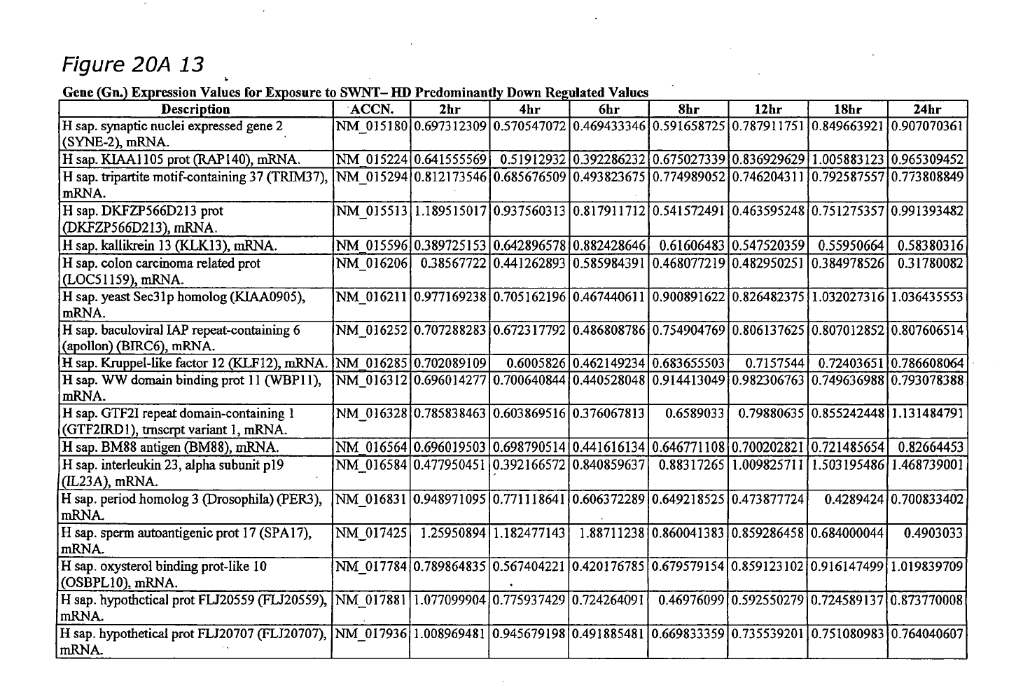

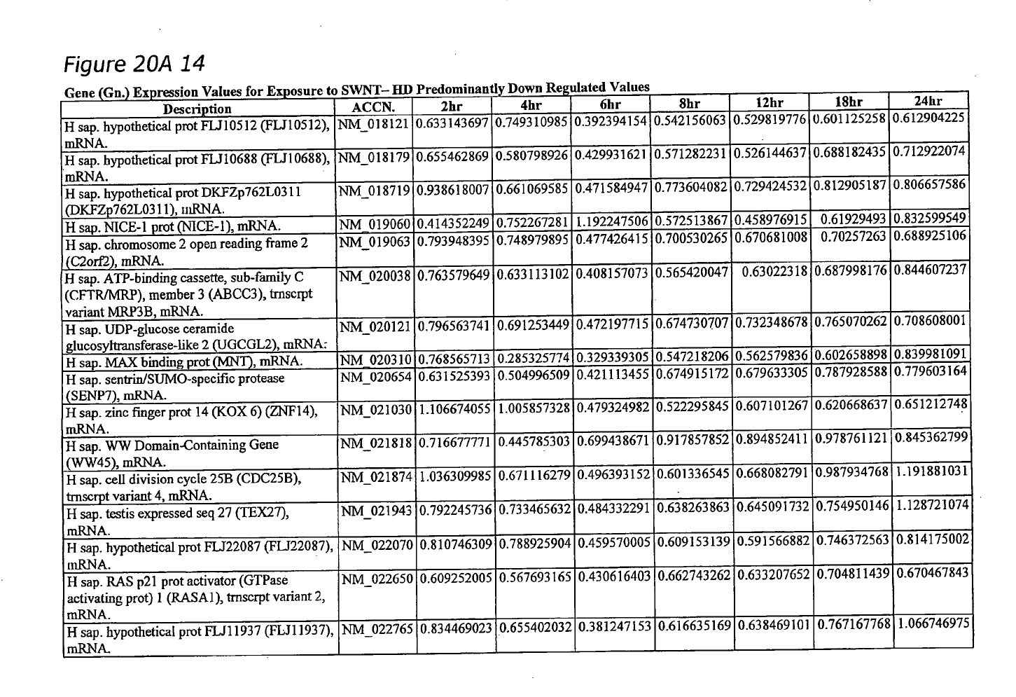

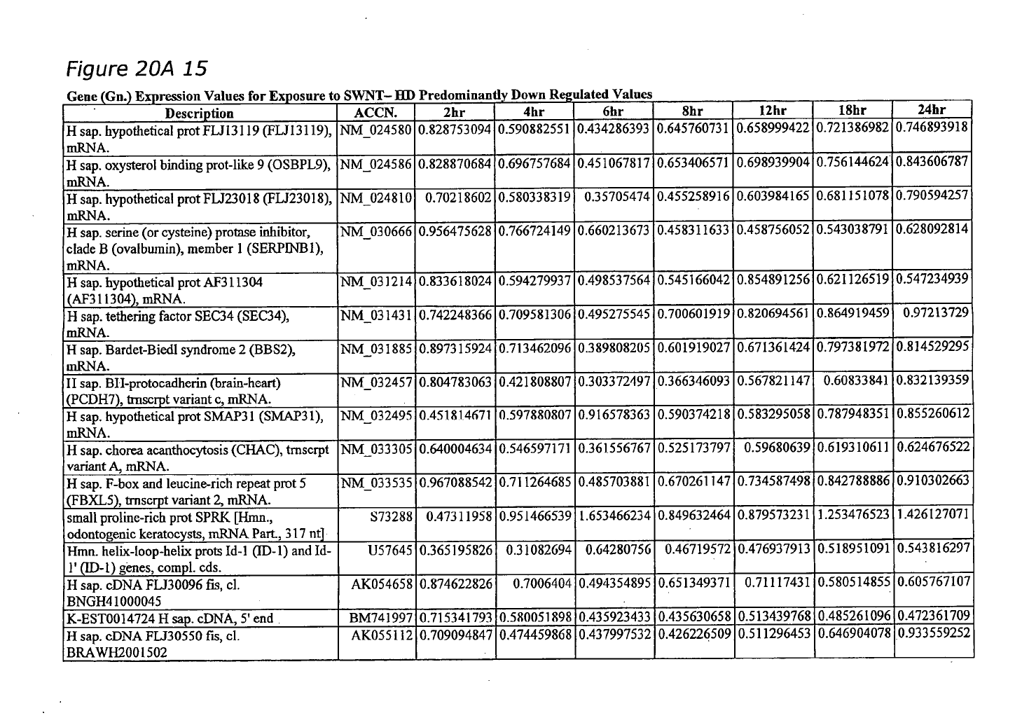

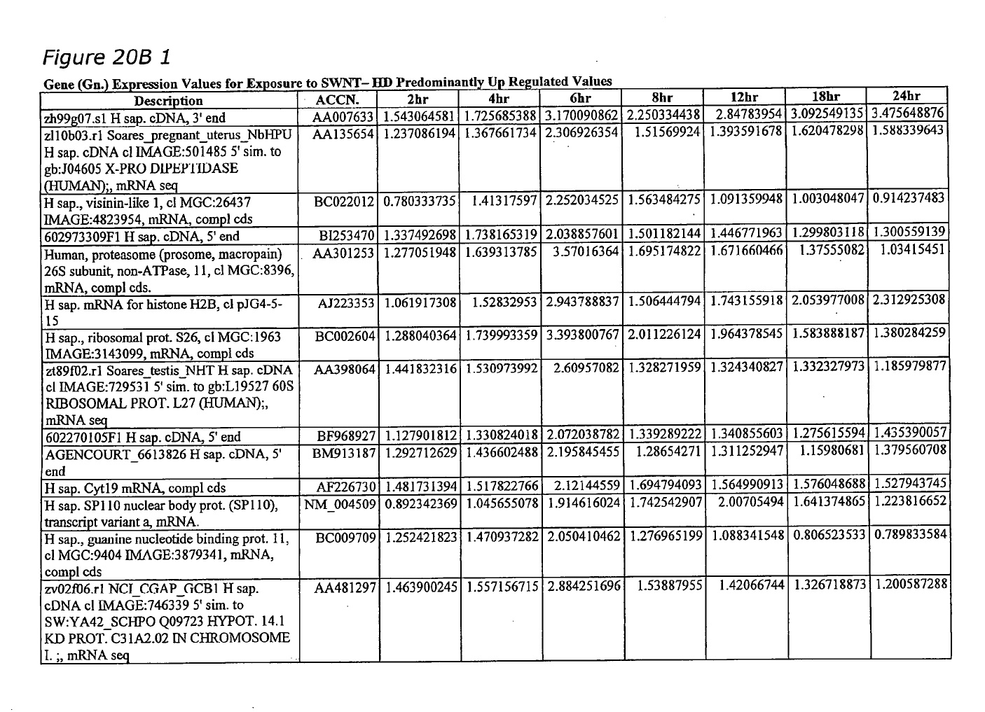

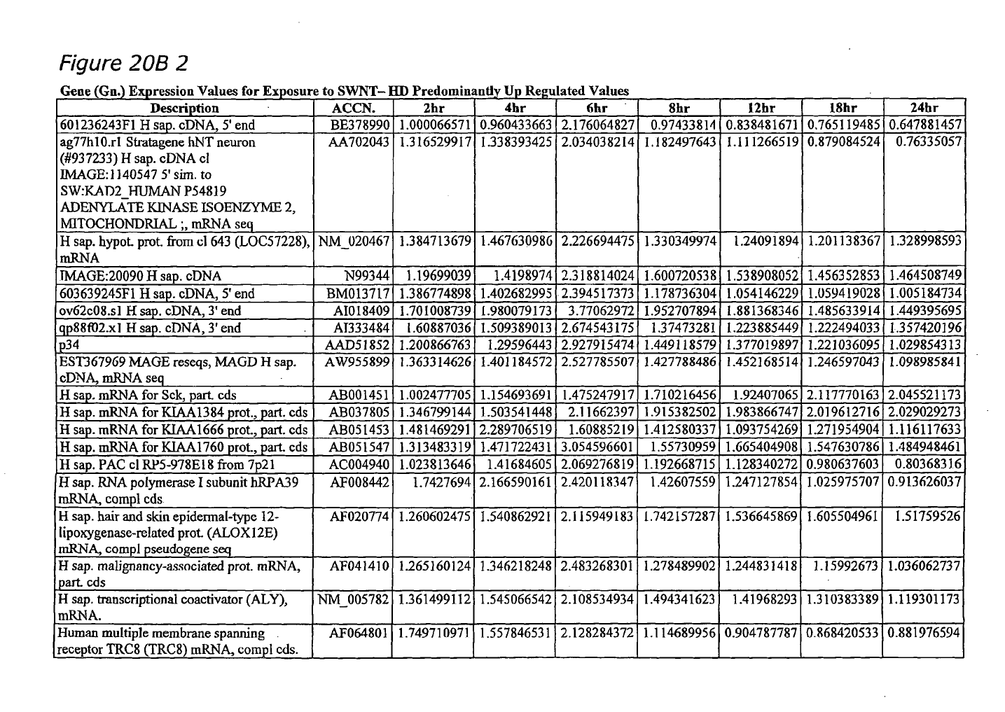

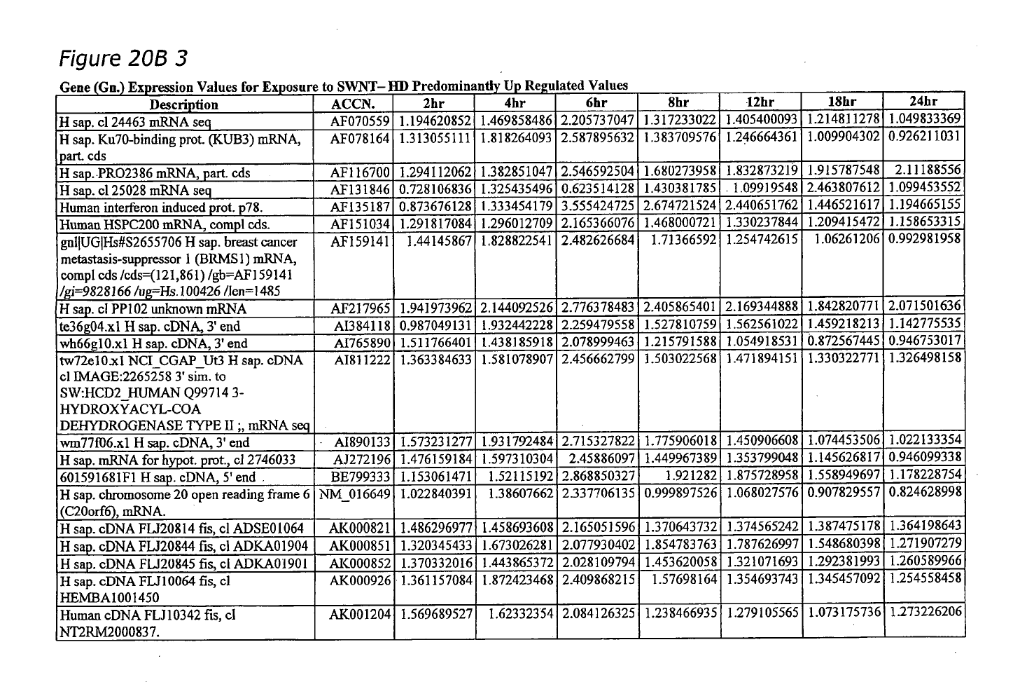

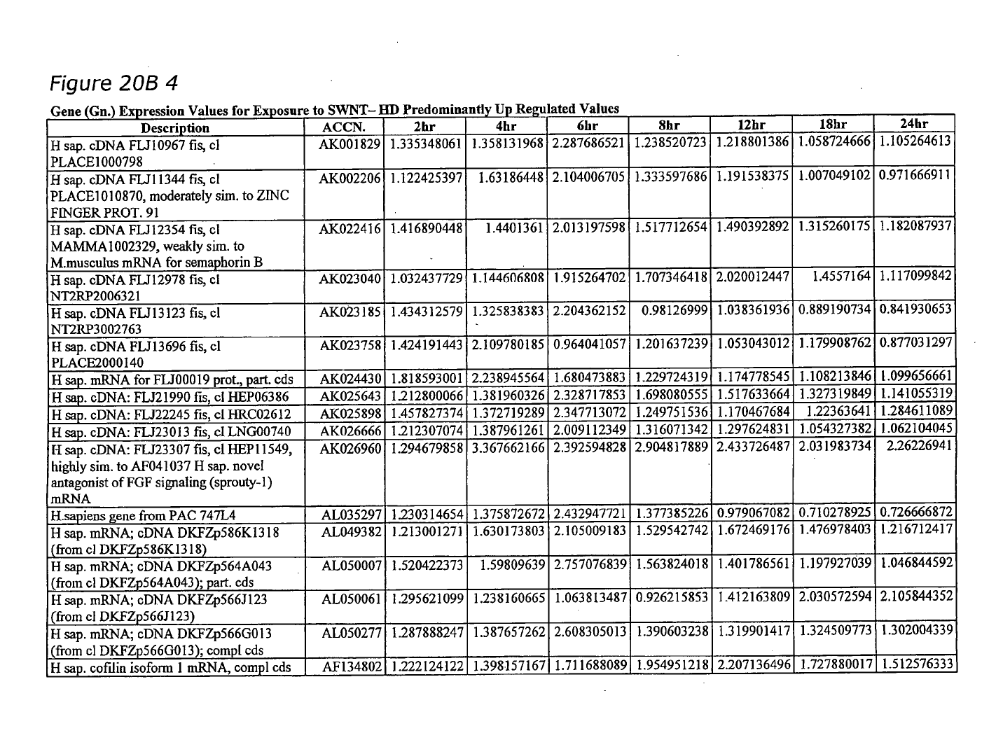

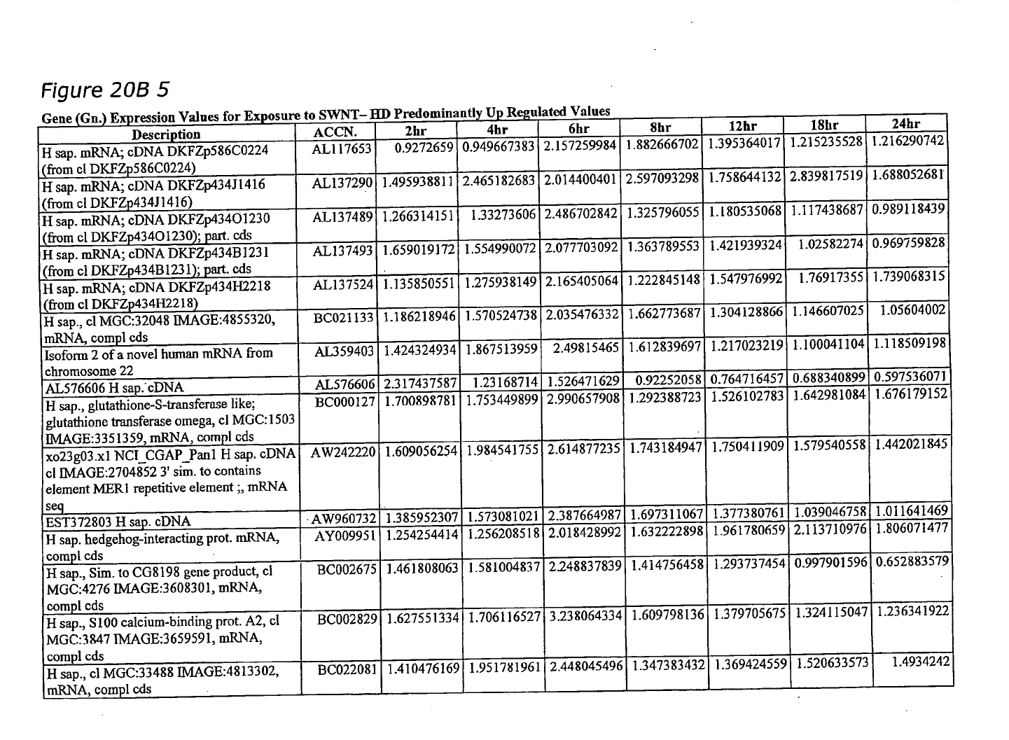

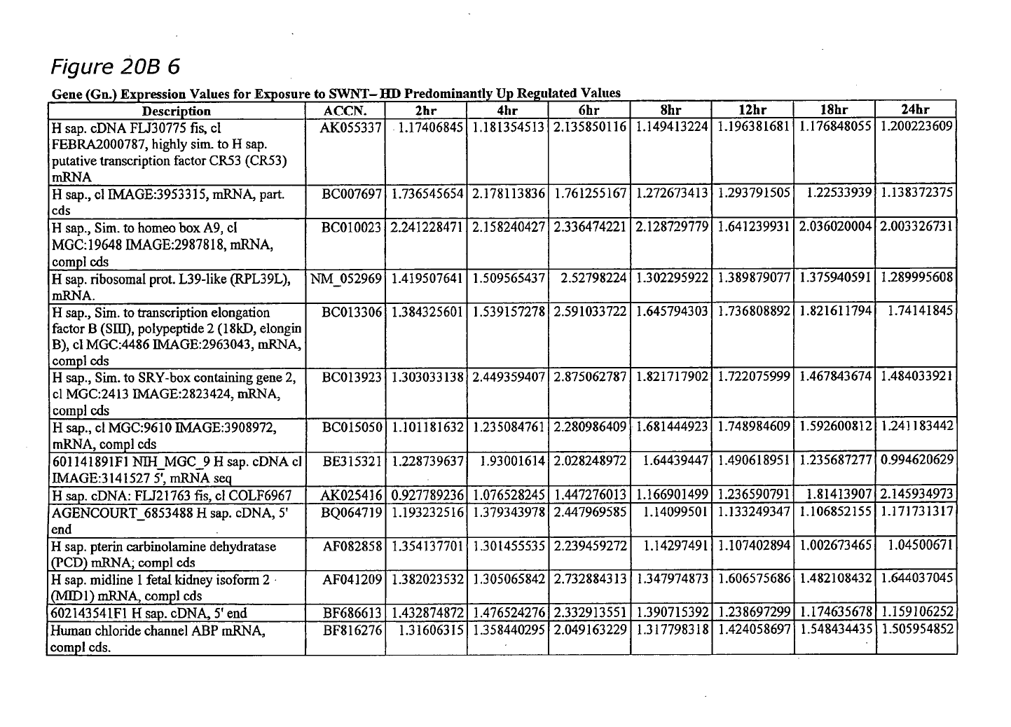

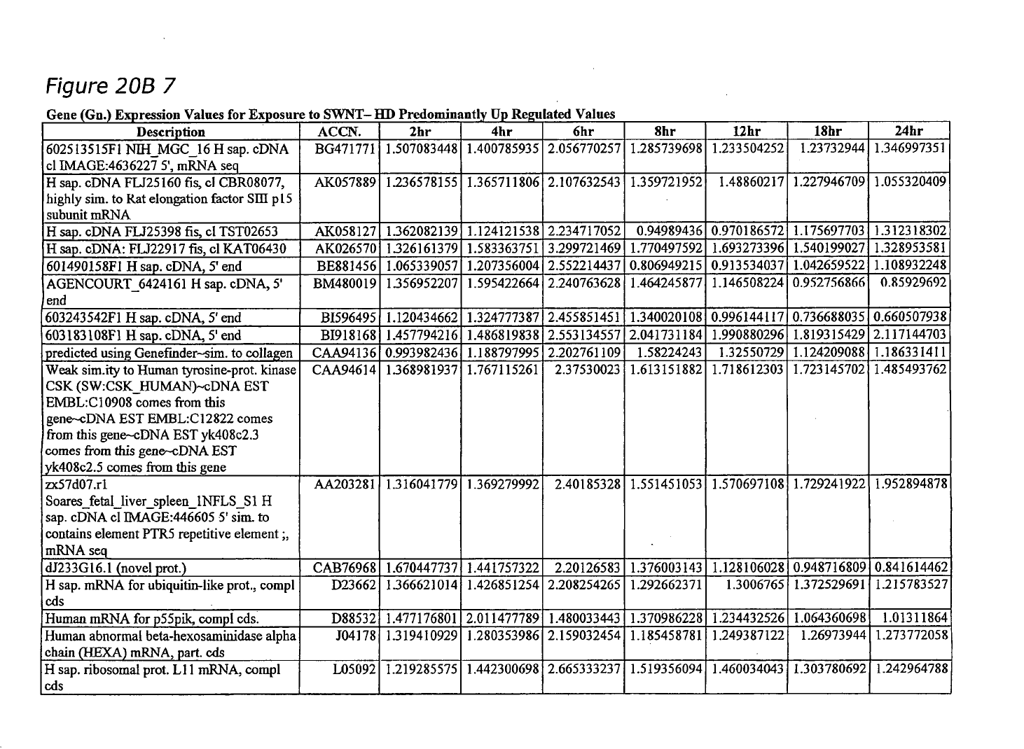

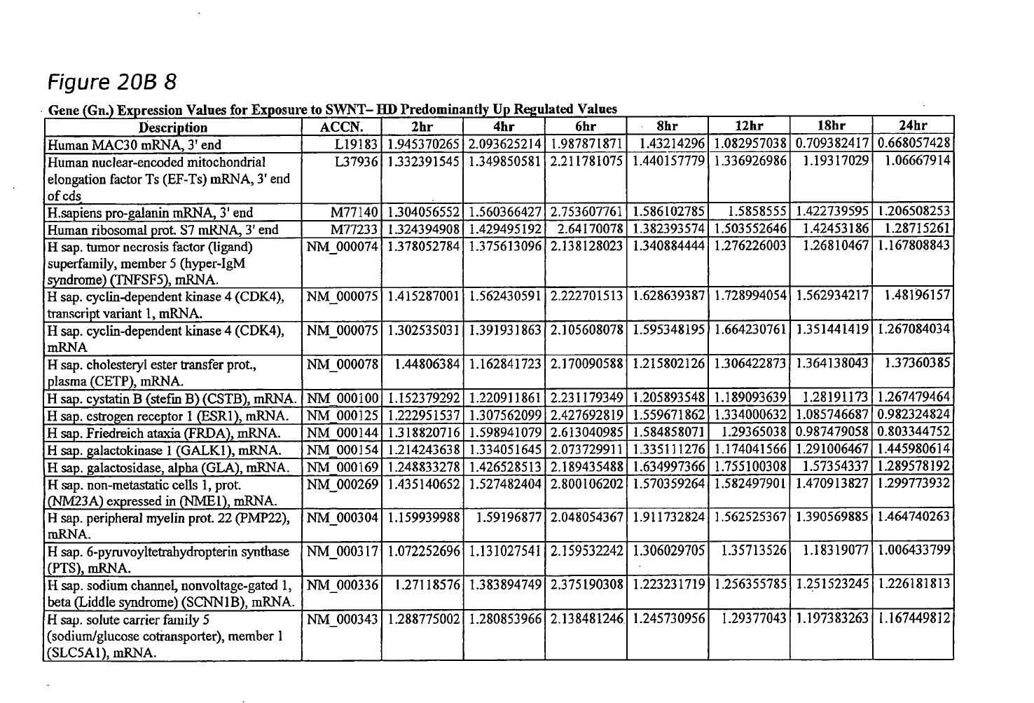

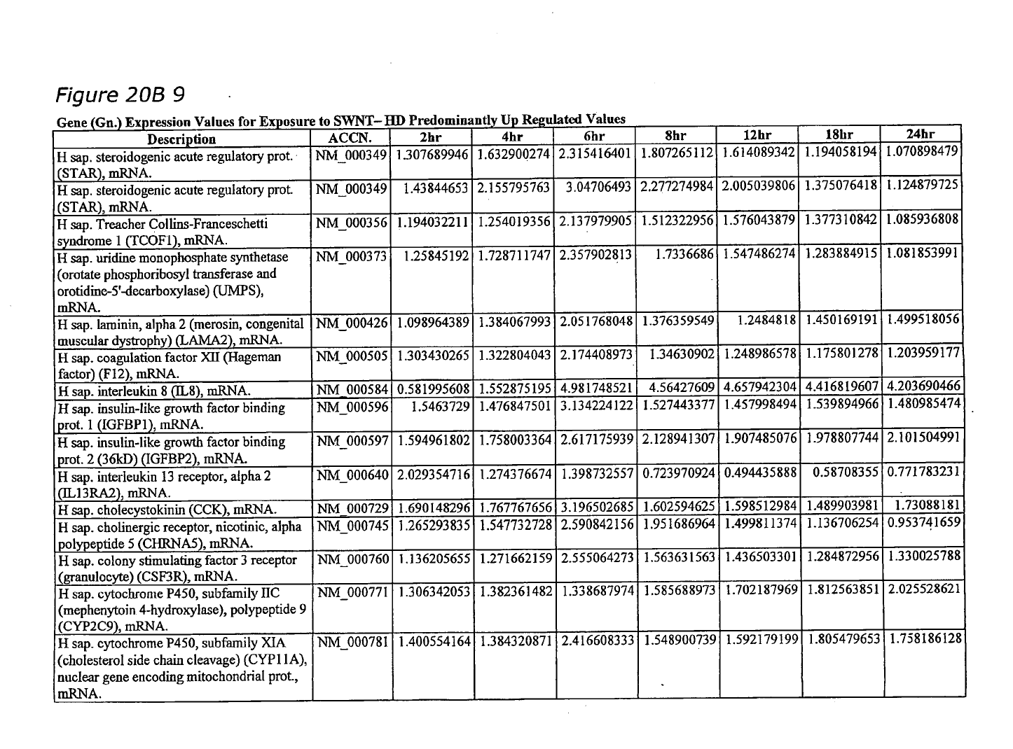

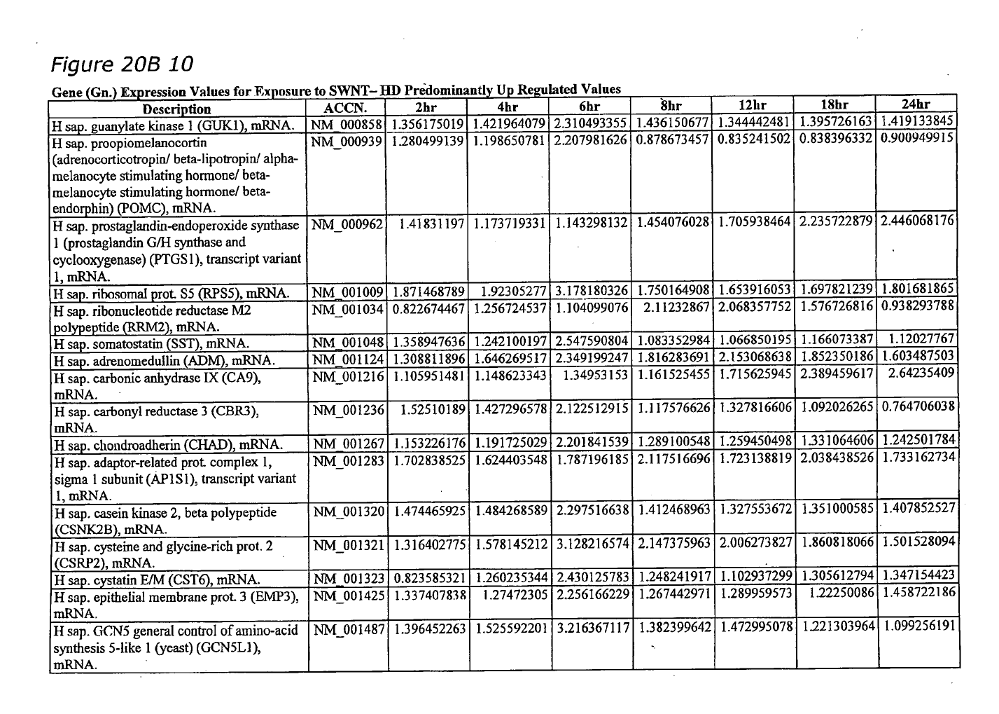

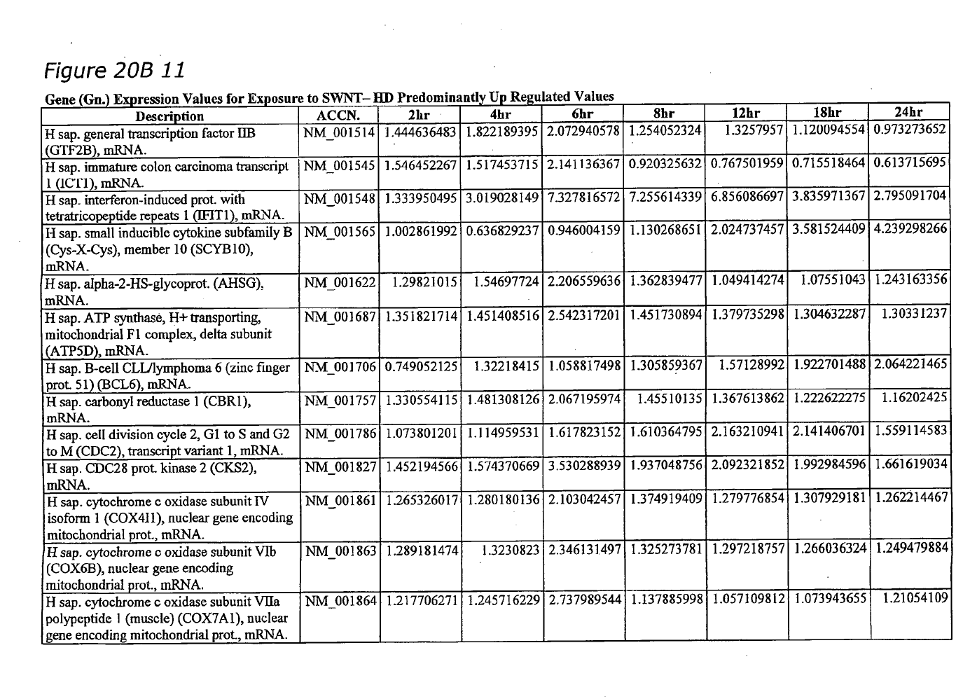

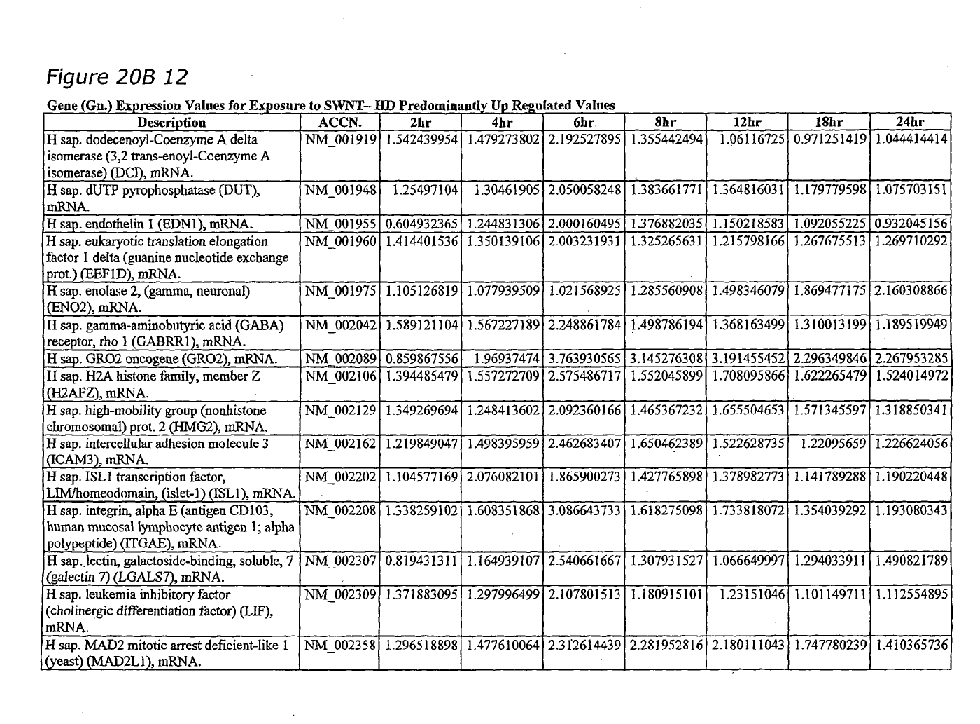

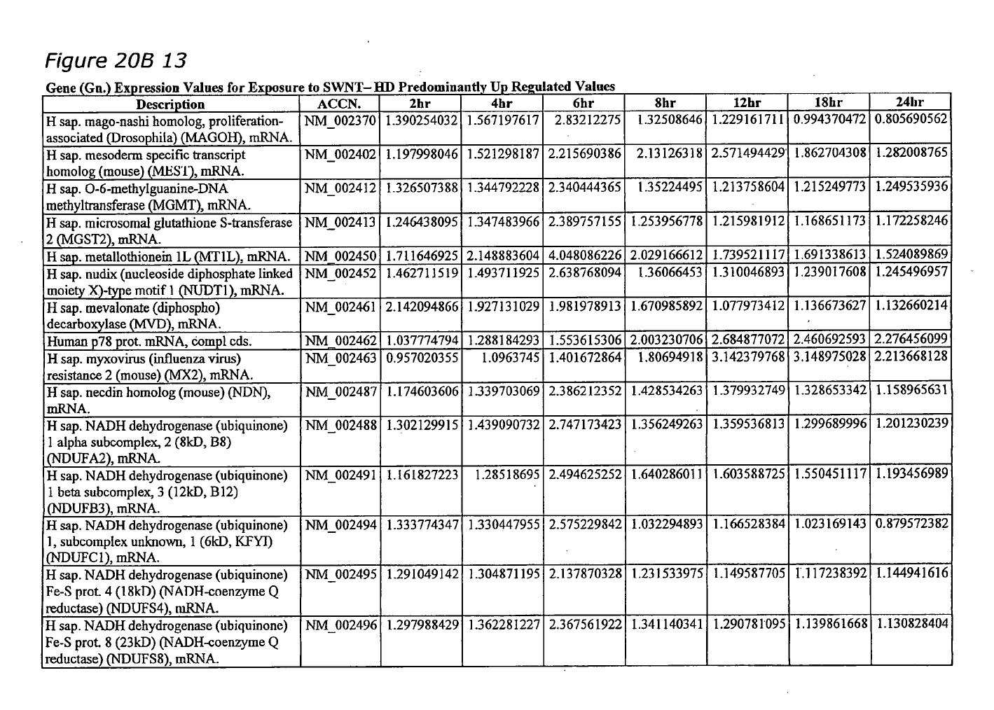

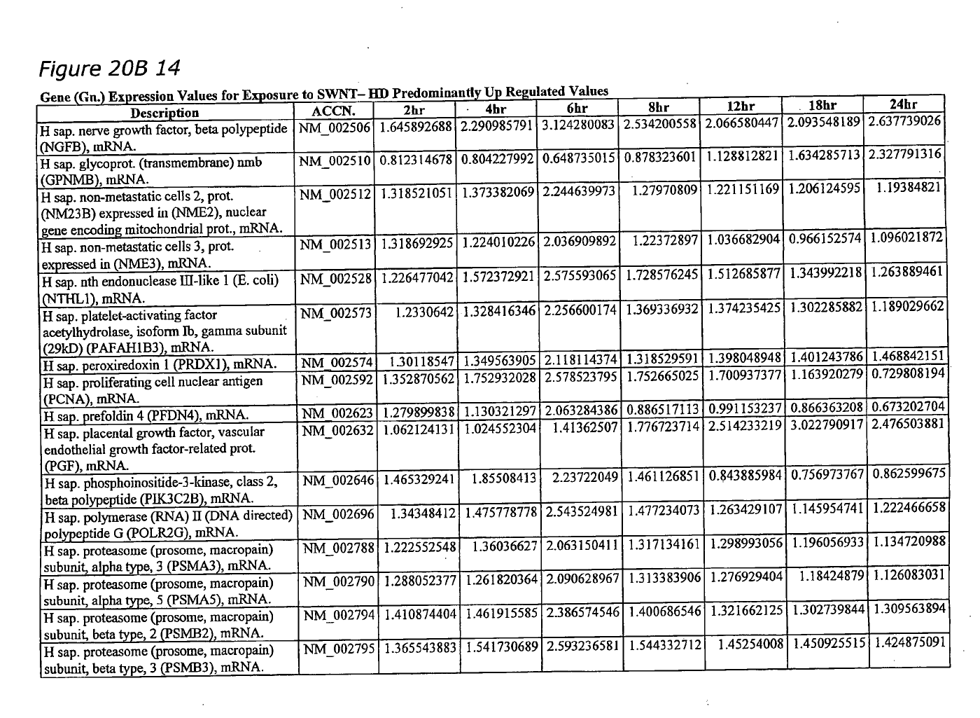

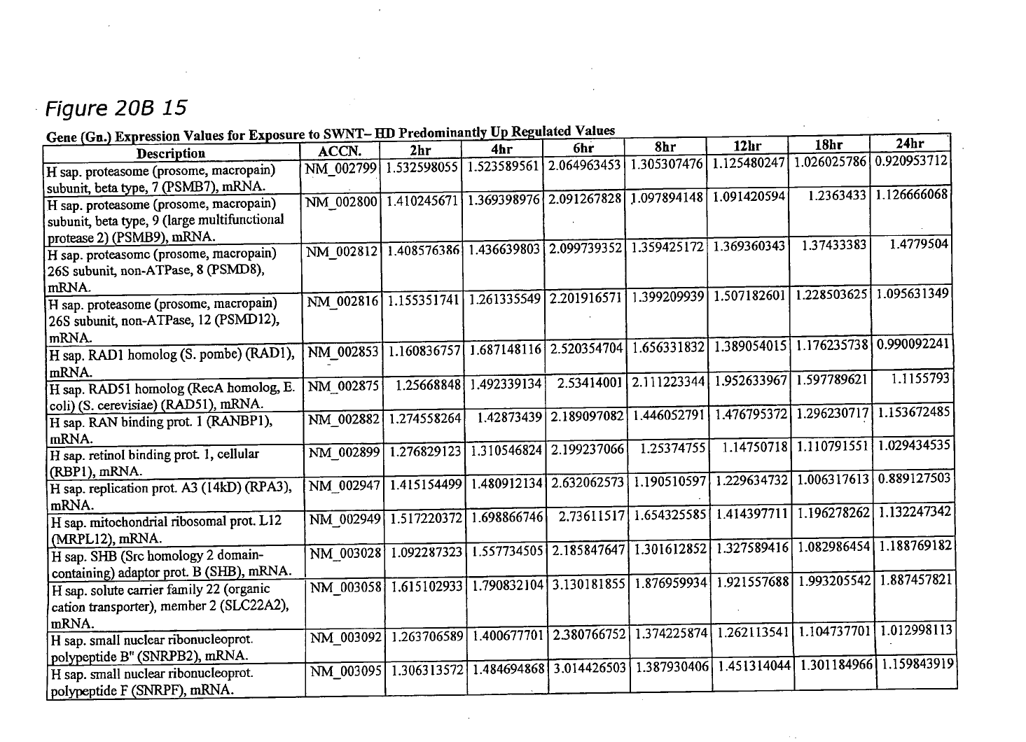

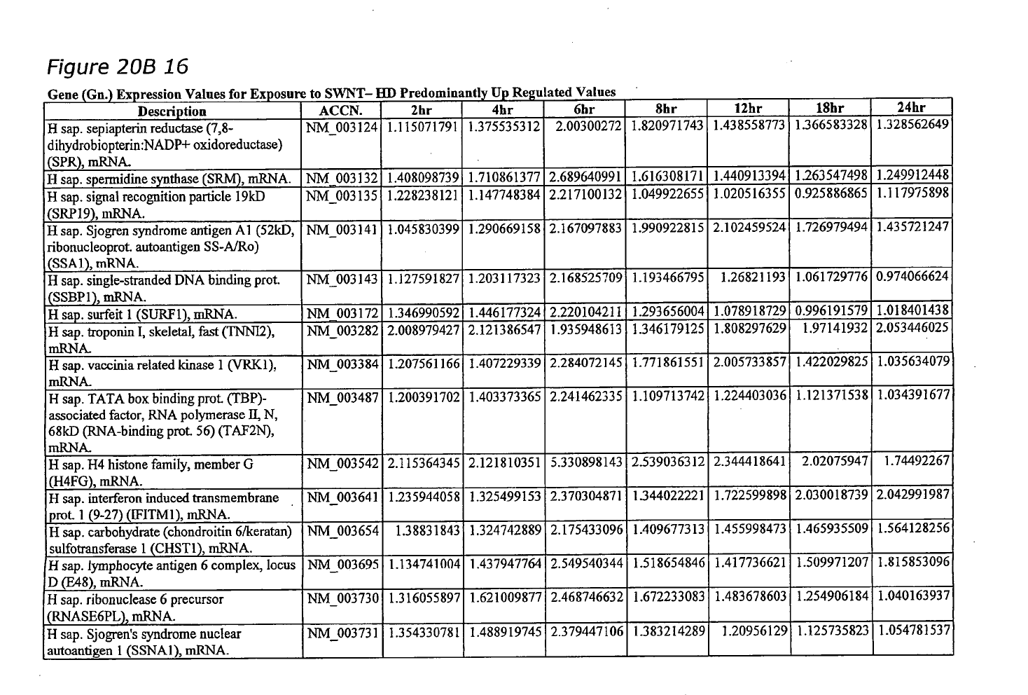

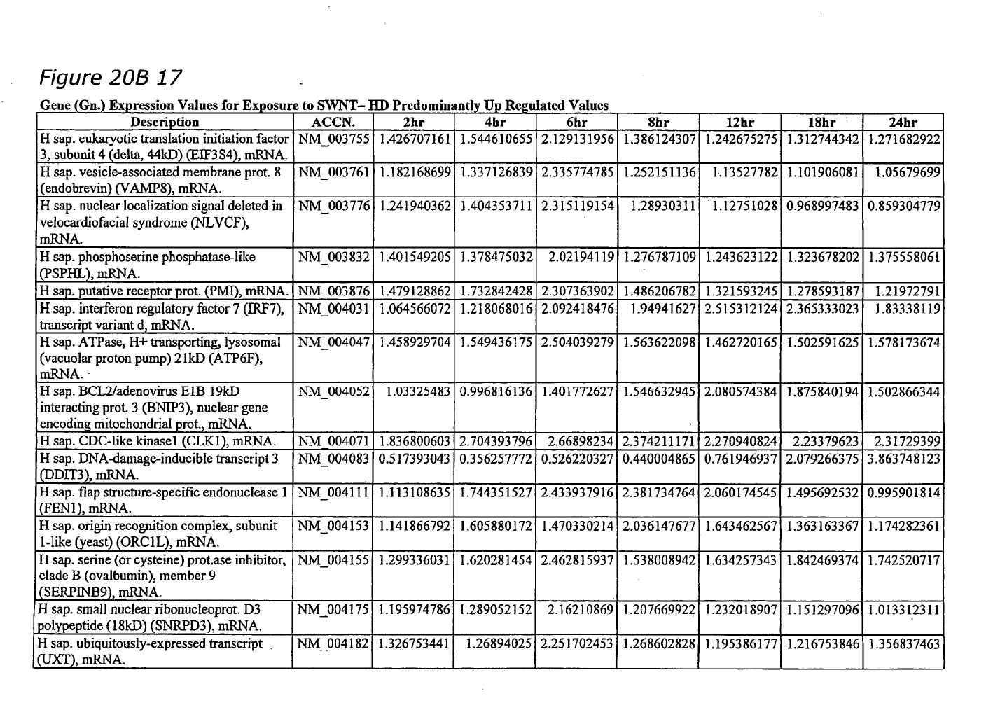

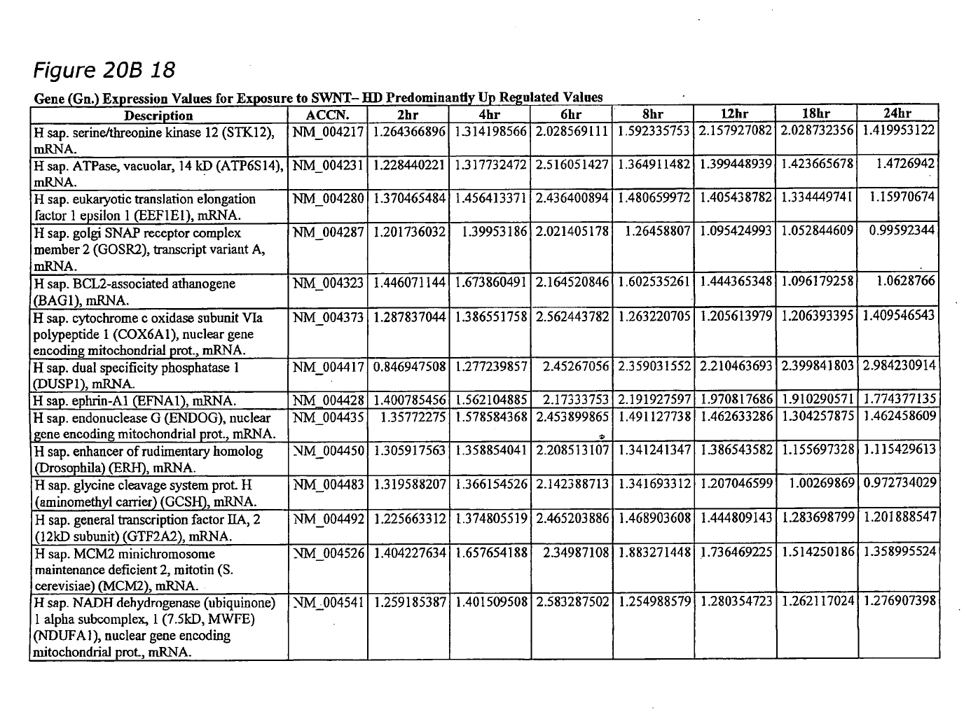

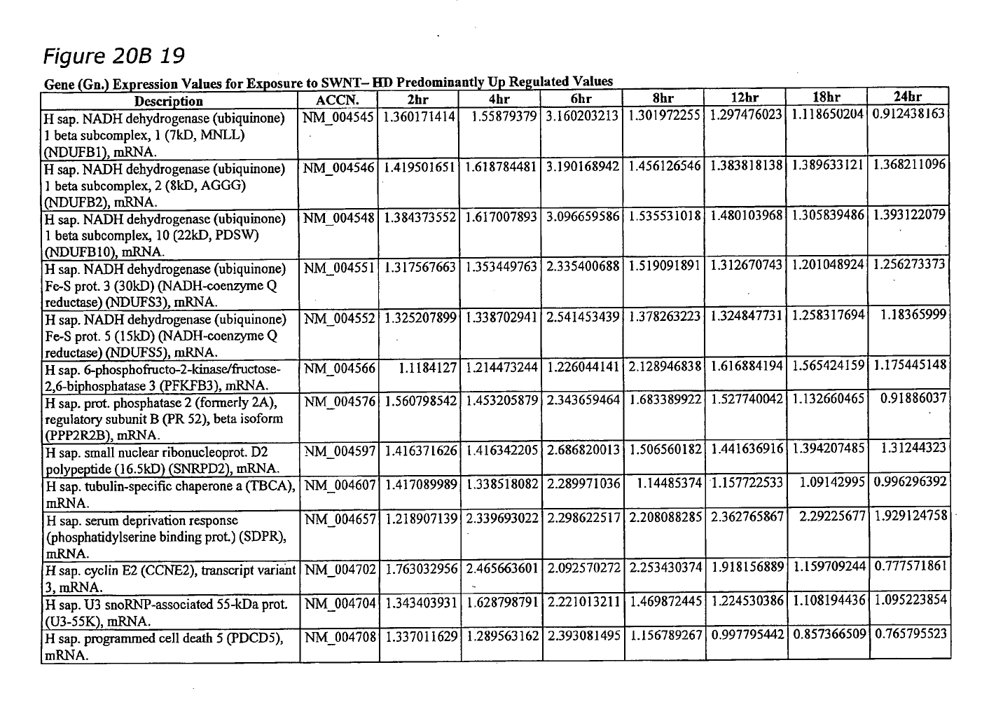

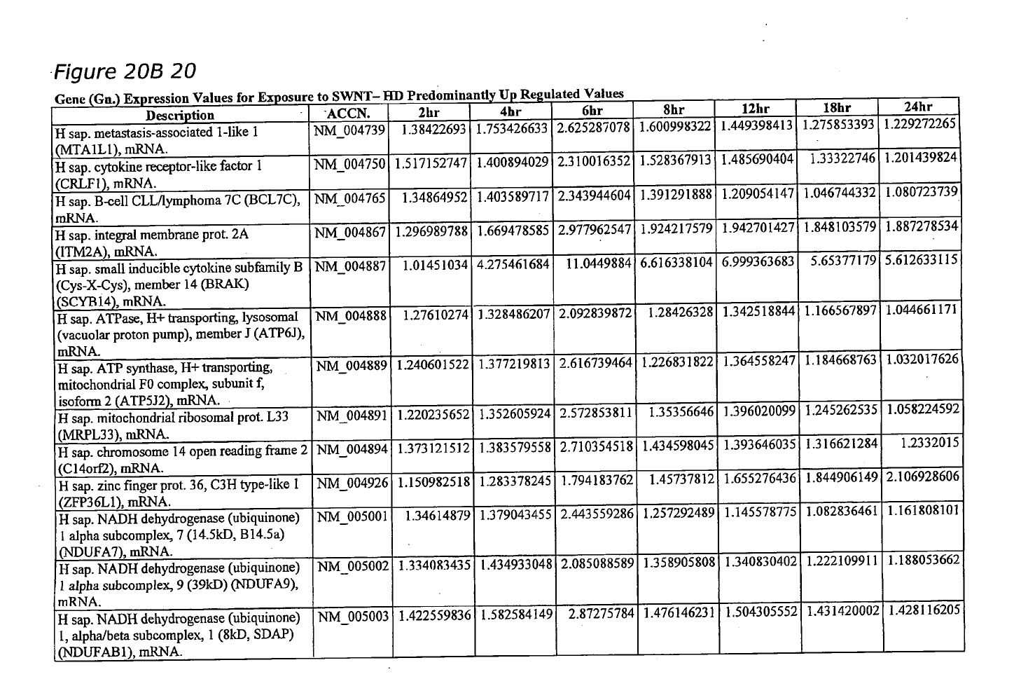

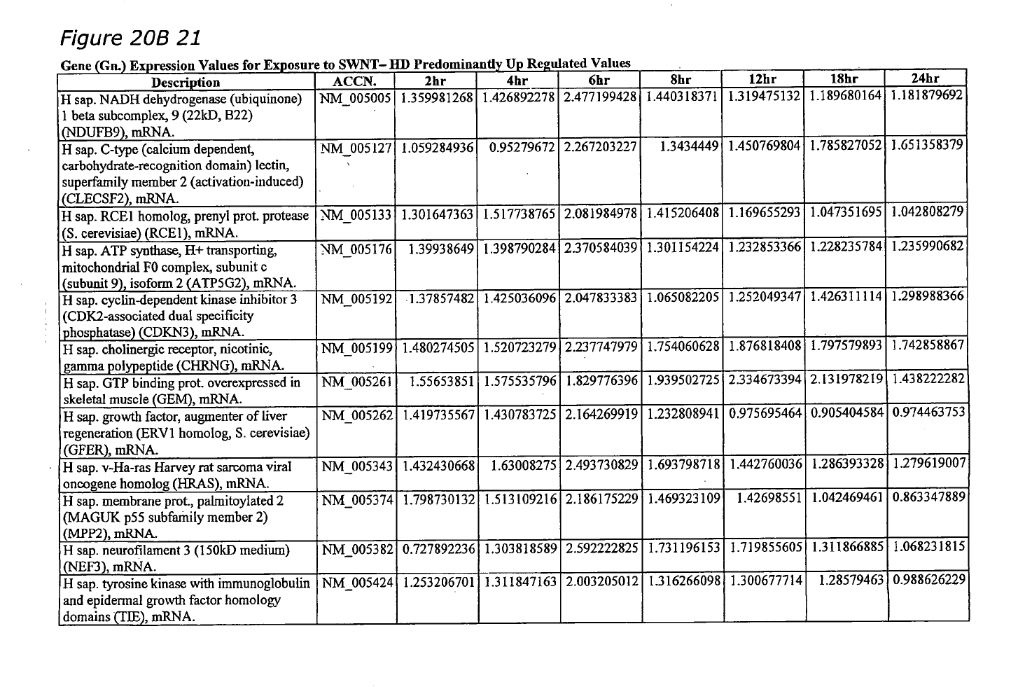

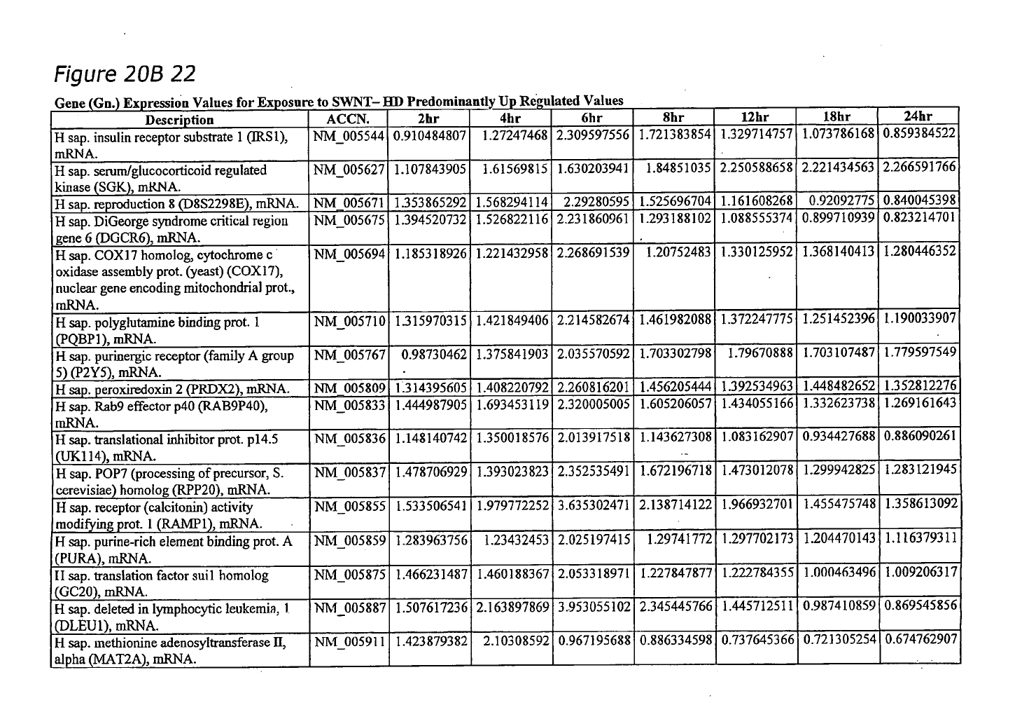

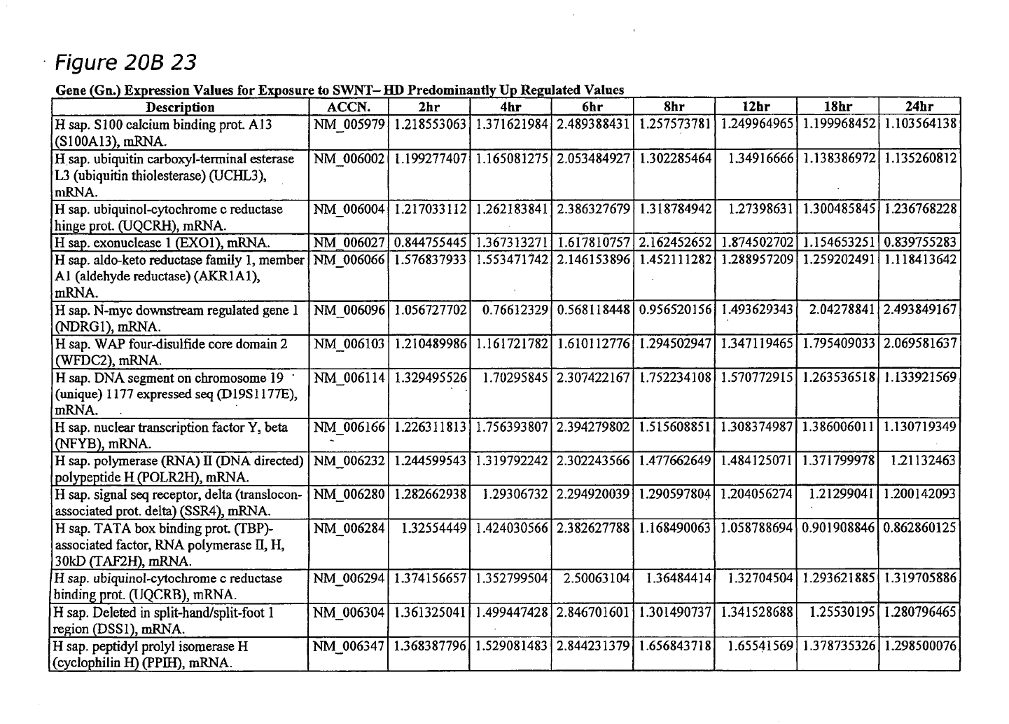

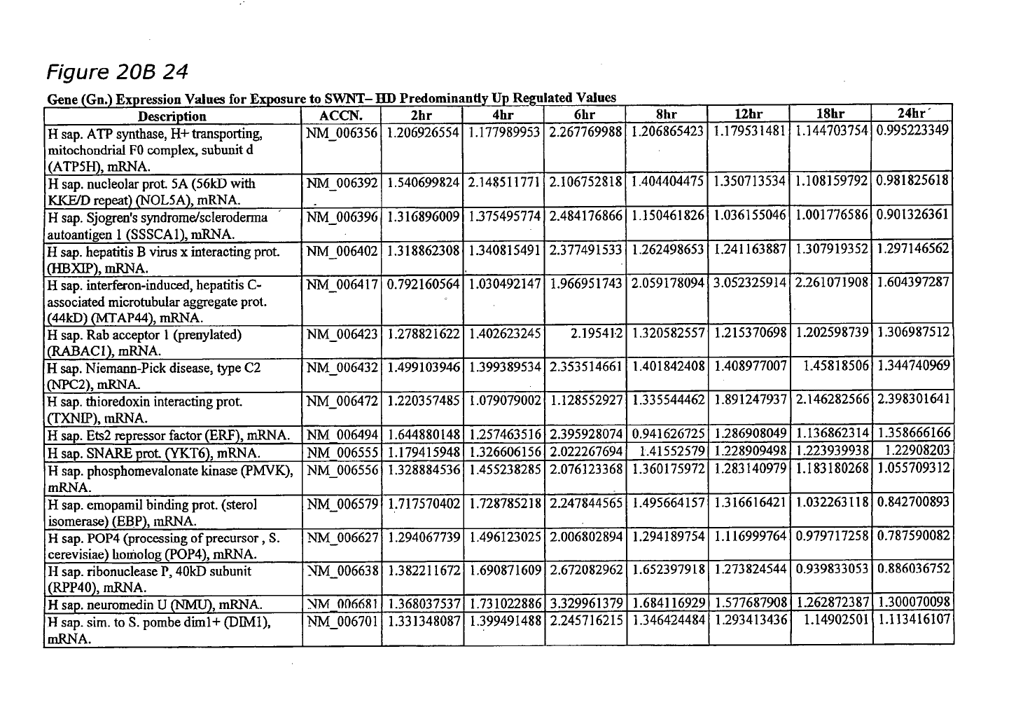

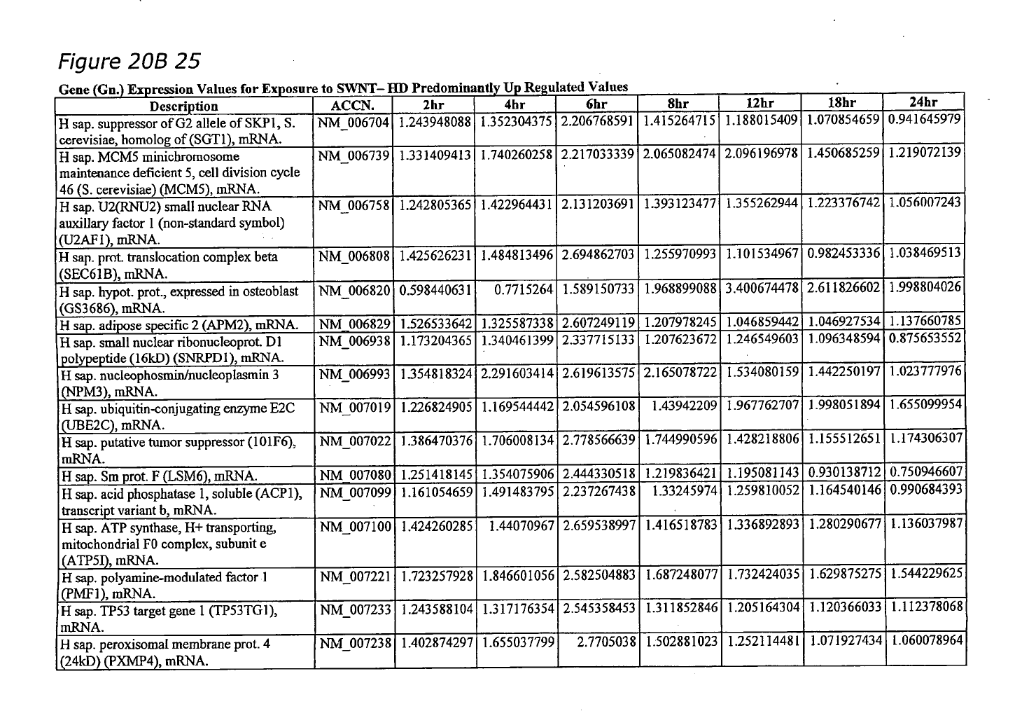

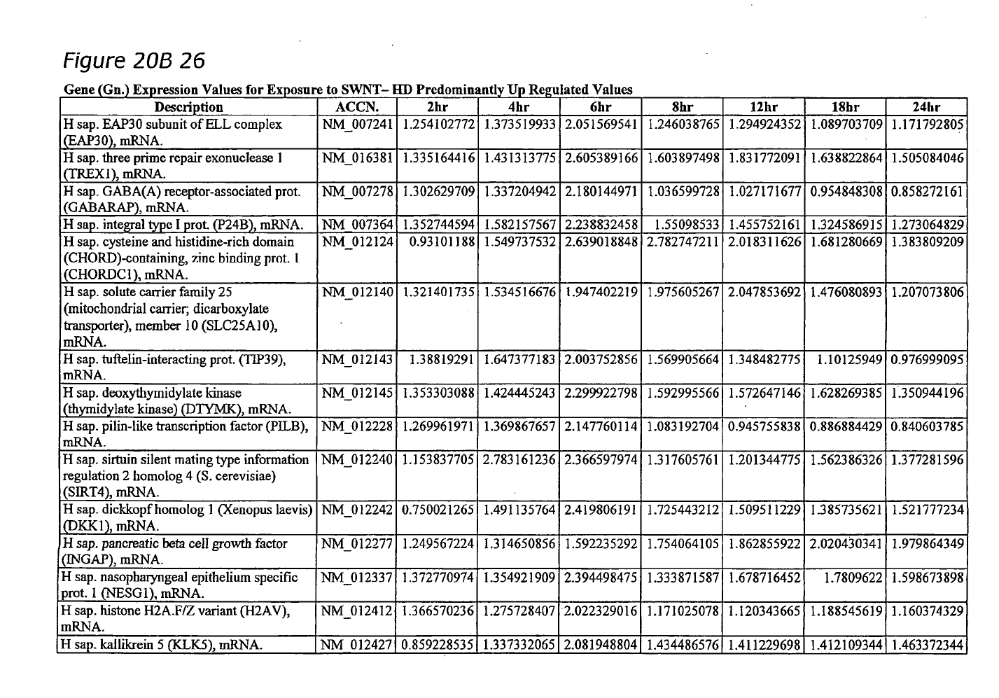

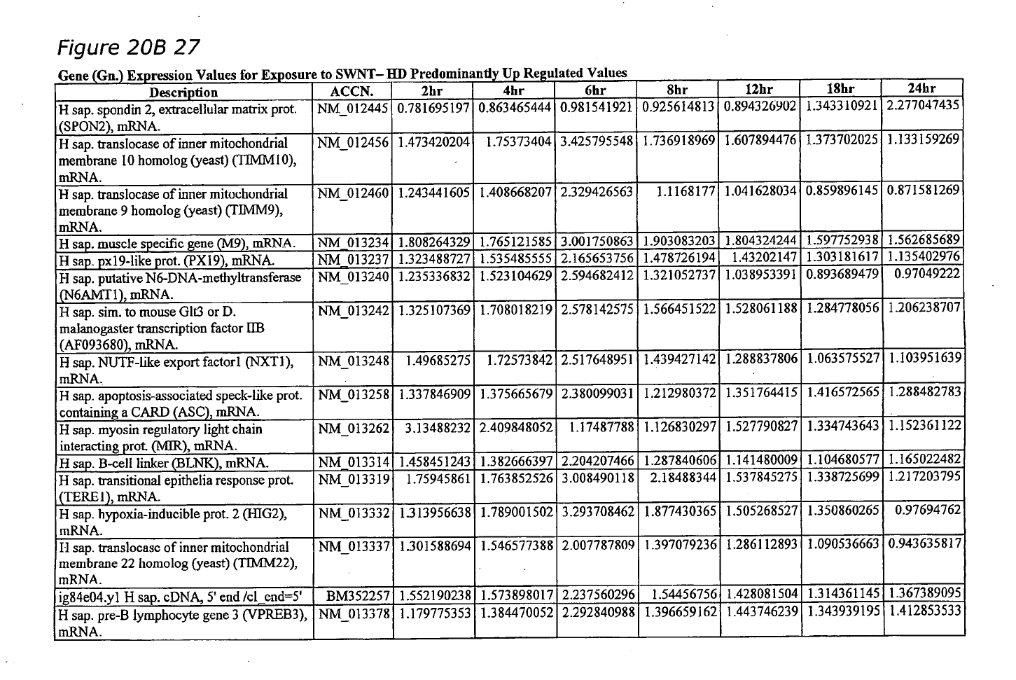

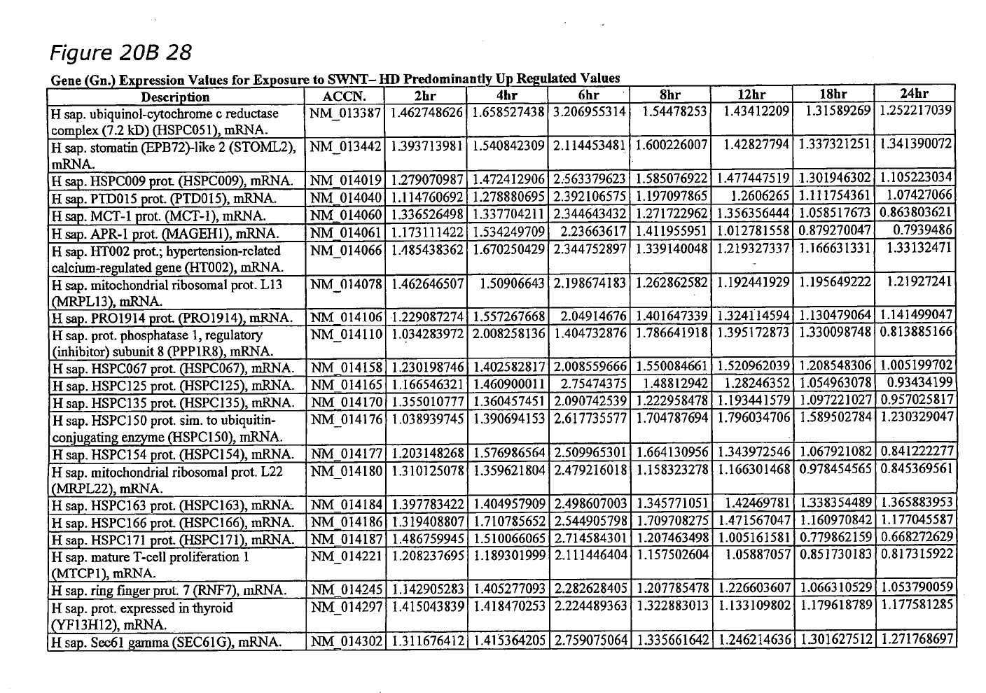

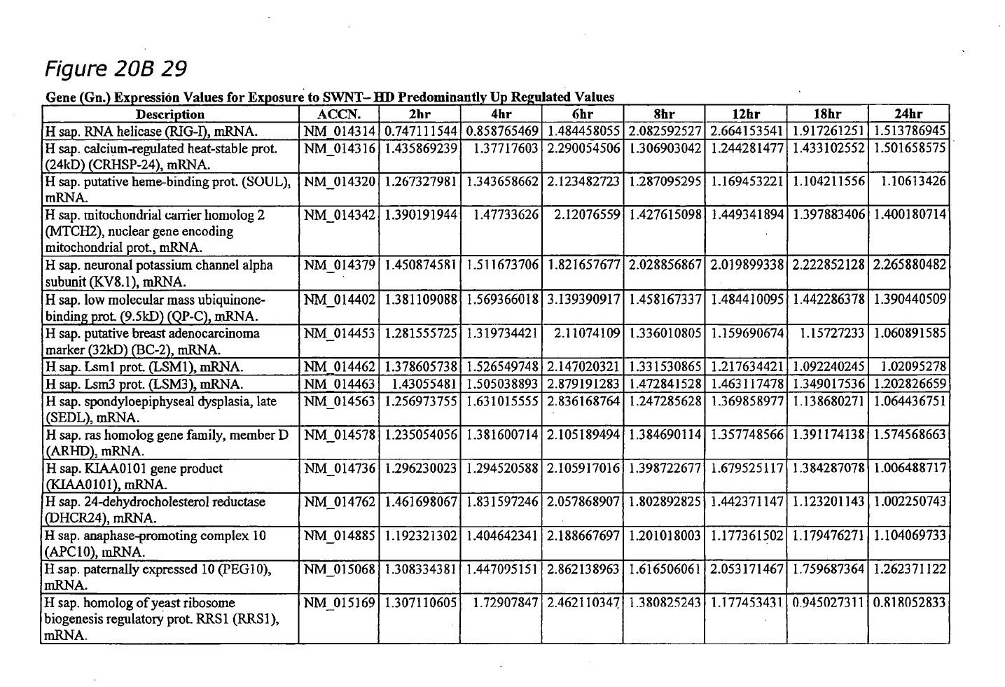

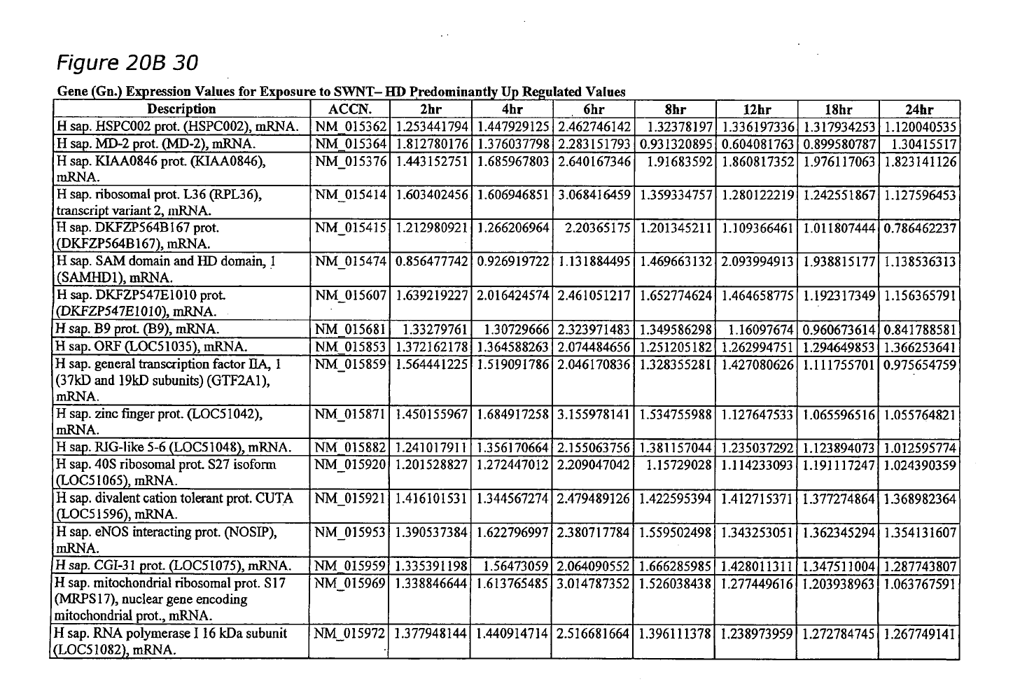

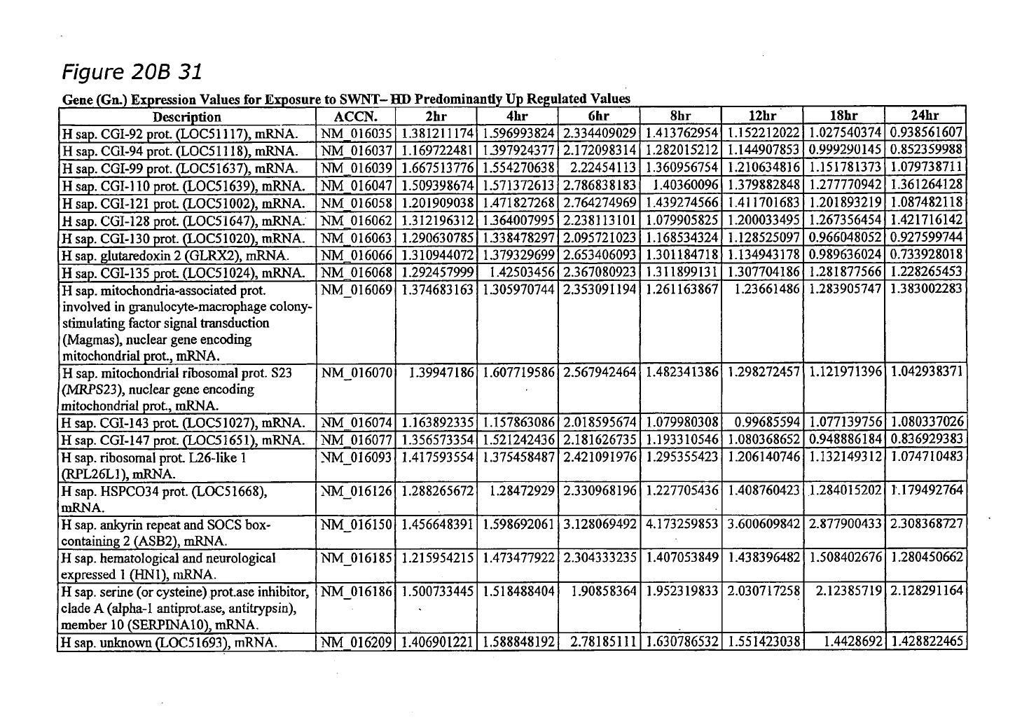

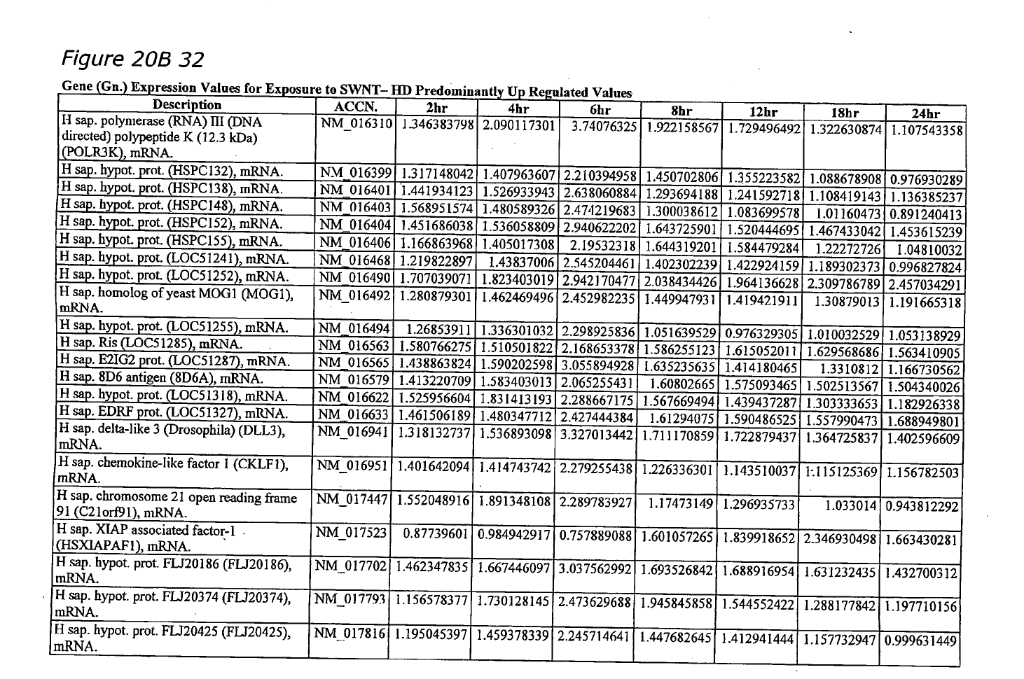

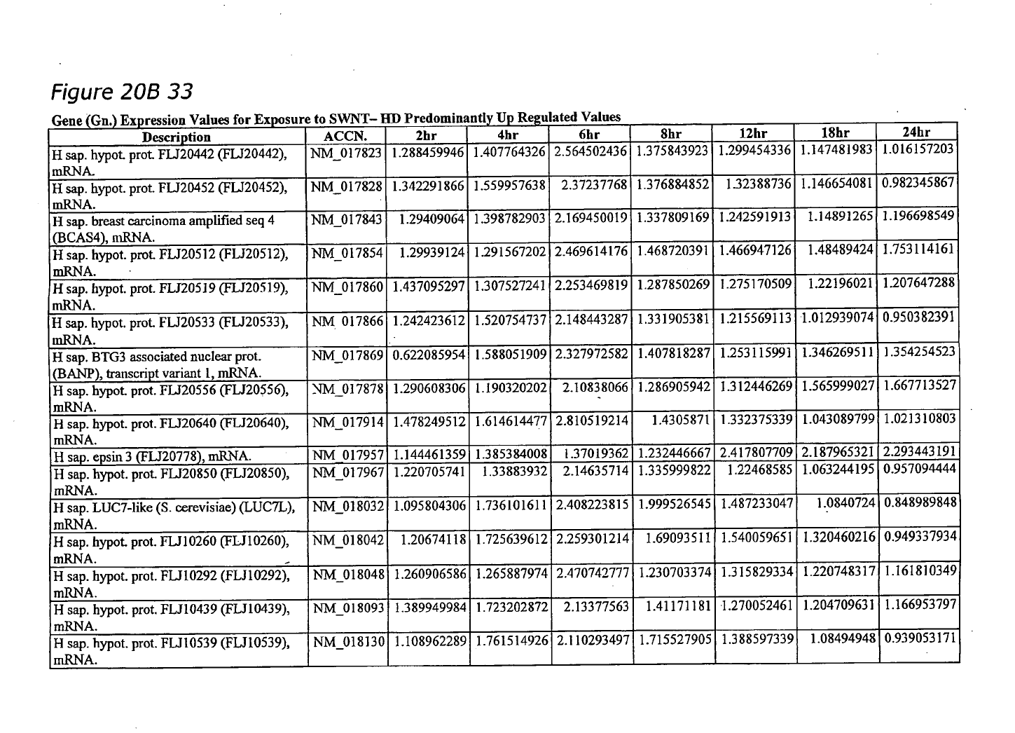

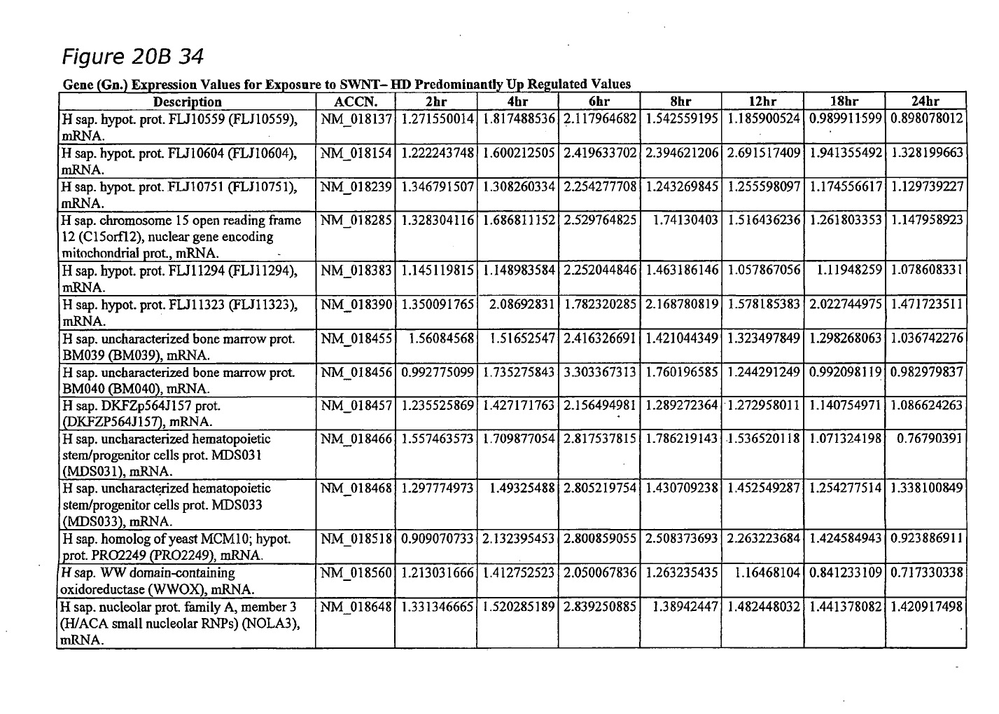

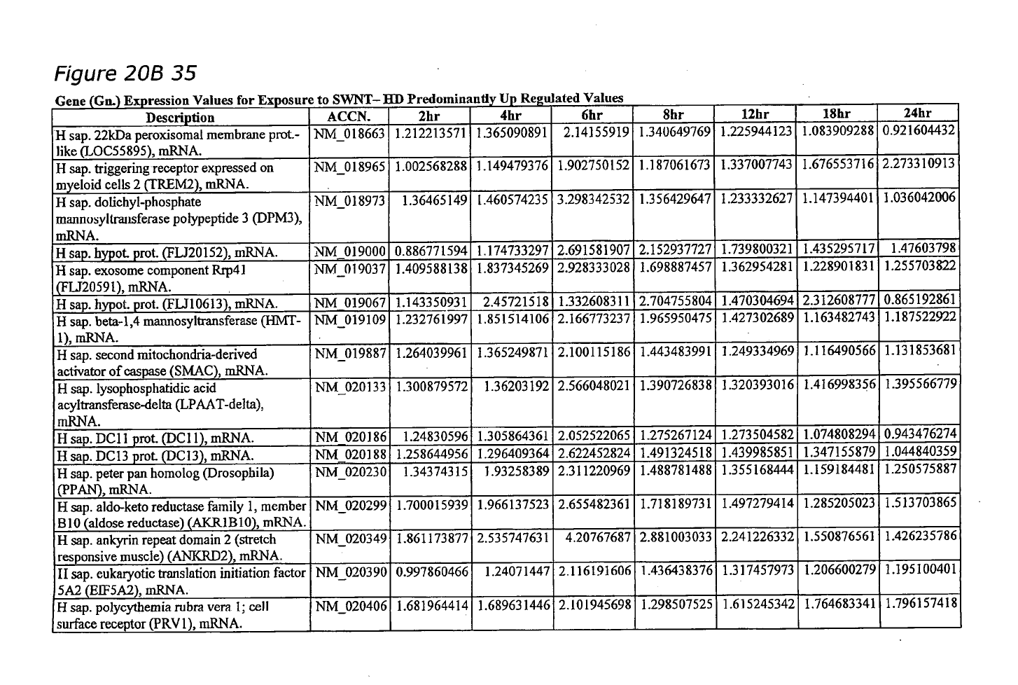

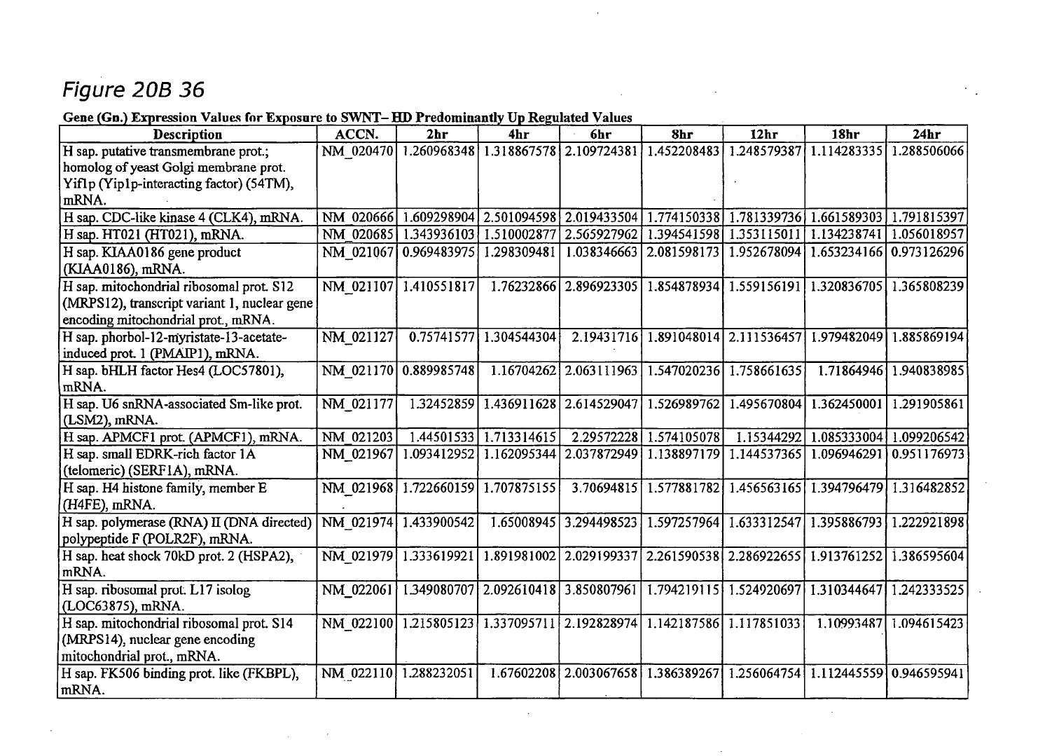

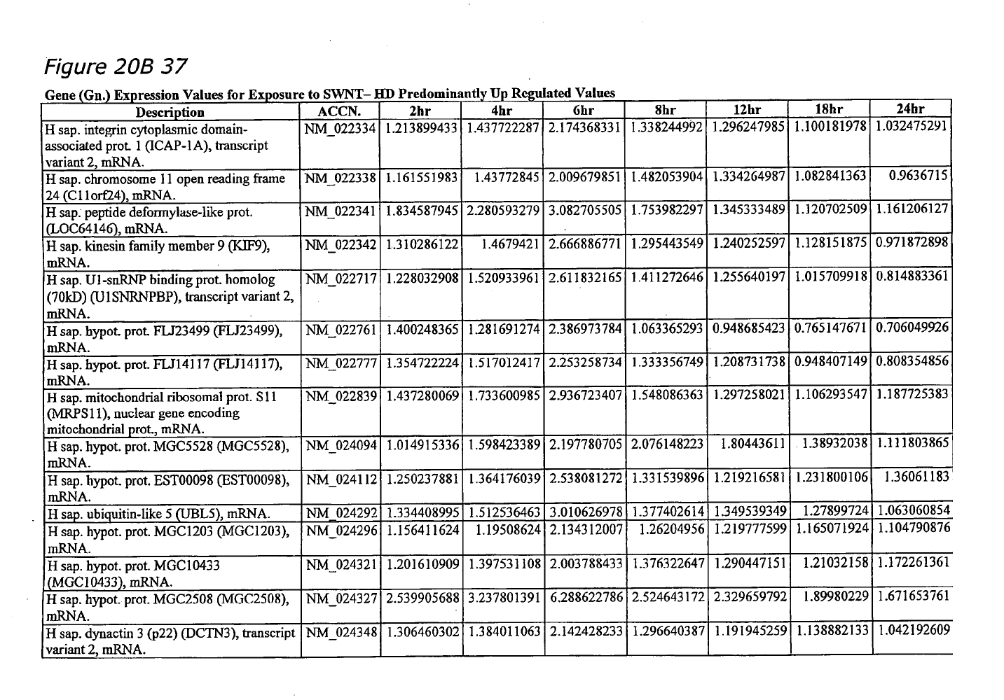

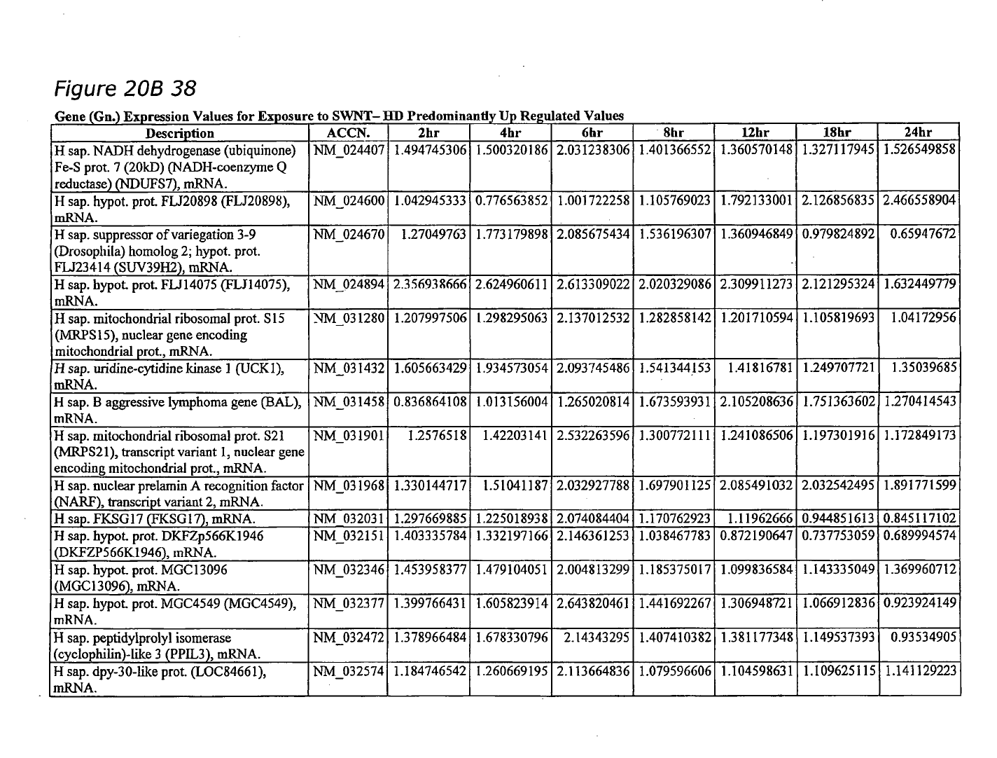

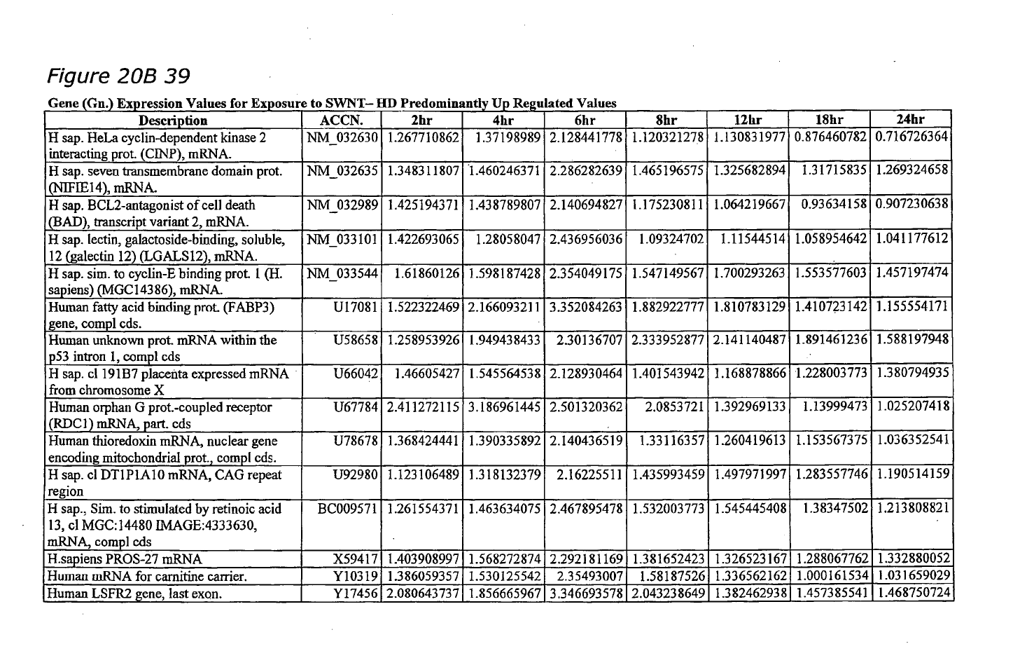

FIG. 20A1-15 presents GEM results for genes predominantly down-regulated in response to high dose SWNT nanoparticle exposure over time in HEK cells.

FIG. 20B1-39 presents GEM results for genes predominantly up-regulated in response to high dose SWNT nanoparticle exposure over time in HEK cells.

IBIS Analysis for LDS and QDA Tables from the CB, FC and SiO2 experiments above:

The data used for the analysis consisted of the normalized intensities (gene expression values) for all microarray probes annotated as “discovery” (non control probes) and with a quality flag of “good” (fluorescent signal for the probe spot on the array conformed to specifications, was not contaminated, irregular or low intensity). The gene expression values are from three microarrays run on the same biological sample (triplicates) according to the MIAME guidelines. The analysis was performed using IBIS (Integrated Bayesian Inference System, GeneLinker Platinum, ver. 4.6.1, Improved Outcomes Software, Inverary, Ontario, Canada), This method separates out genes which are predictive of specific class memberships (variables, user-specified). In this case, the variables were set to nontoxic, low toxicity and high toxicity. Two types of classifiers were used: linear discriminant analysis (LDA) and quadratic discriminant analysis (QDA) in one dimension. The parameters used were 10 committee members (using a modification of artificial neural networks), 66% of committee member votes required, and the random seed set to 999. In addition, the minimum standard deviation is set by the software to the appropriate smallest standard deviation of expression for any gene/sample pair over a number of replicate measurements for each data set analyzed.

The tabular results include gene description, gene ascension number, accuracy and mean squared error. The accuracy is how well the gene is able to be used as a discriminator and varies from 0-100%. The mean squared error (MSE) reflects the level to which the data matches the linear or quadratic model with lower values being the best.

It is in the pattern of expression over time that these genes can provide discrimination as to overall toxicity regardless of the compound. Thus, these genes are effectively stress indicators. FIG. 15A and B represents LDA Analysis of the data of FIGS. 12 (TiO₂), 13 (CB) and 14 (SiO₂)

FIG. 16A-D represents QDA Analysis of the data of FIGS. 12 (TiO₂), 13 (CB) and 14 (SiO₂)

The following LDA and QDA tables identify those markers that discriminate between high, low and non-toxic exposure at both high and low dose exposure. The toxicity responses are a surrogate for identification of the compounds based on their inherent toxicity: SiO₂ is defined based on the historical literature as high toxic, TiO₂ and CB are defined as low-toxic while ferronyl iron (AKA, FC or carbonyl iron) is defined as as non-toxic.

LDA 1D Low Dose

| | | Accuracy | Mean Squared |
|---|---|---|---|
| Description | ACCN | % | Error |
| Homo sapiens cDNA | AK001803 | 88 | 6.493E−2 |
| FLJ10941 fis, clone | | | |
| OVARC1001243 | | | |

QDA 1D Low Dose

| | | Accuracy | Mean Squared |
|---|---|---|---|
| Description | ACCN | % | Error |
| Homo sapiens cDNA | AK001803 | 88 | 5.817E−2 |
| FLJ10941 fis, clone | | | |
| OVARC1001243 | | | |

LDA 1D High Dose

| | | | Mean |
|---|---|---|---|
| | | Accuracy | Squared |
| Description | ACCN | % | Error |
| Homo sapiens neurofibromin 1 | NM_000267 | 93 | 4.352E−2 |
| (neurofibromatosis, von | | | |
| Recklinghausen disease, | | | |
| Watson disease) (NF1), mRNA. | | | |
| Homo sapiens CDC-like kinase1 | NM_004071 | 92 | 4.473E−2 |
| (CLK1), mRNA. | | | |
| Homo sapiens mRNA; cDNA | AL390214 | 96 | 6.323E−2 |
| DKFZp564O2423 (from clone | | | |
| DKFZp564O2423) | | | |

QDA 1D High Dose

| | | | Mean |
|---|---|---|---|
| | | Accuracy | Squared |
| Description | ACCN | % | Error |
| Homo sapiens mRNA for | AB014524 | 96 | 2.769E−2 |
| KIAA0624 protein, partial cds | | | |
| Homo sapiens cDNA: FLJ22917 | AK026570 | 93 | 2.612E−2 |
| fis, clone KAT06430 | | | |
| Homo sapiens mRNA; cDNA | AL390214 | 93 | 3.859E−2 |
| DKFZp564O2423 (from clone | | | |
| DKFZp564O2423) | | | |
| Homo sapiens neurofibromin 1 | NM_000267 | 93 | 4.36E−2 |
| (neurofibromatosis, von | | | |
| Recklinghausen disease, | | | |
| Watson disease) (NF1), mRNA. | | | |

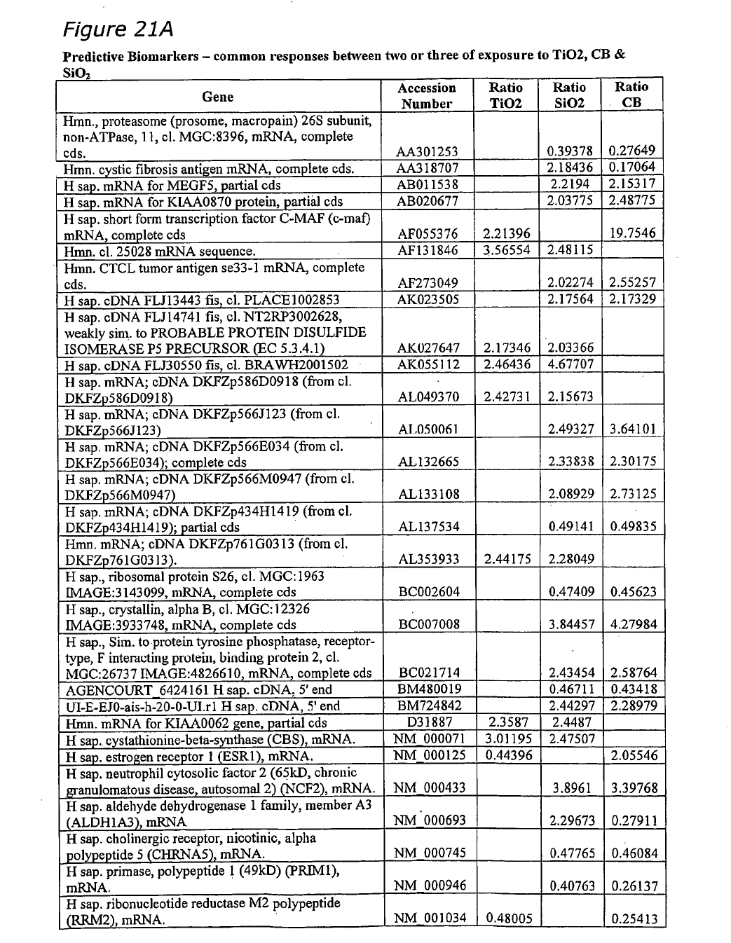

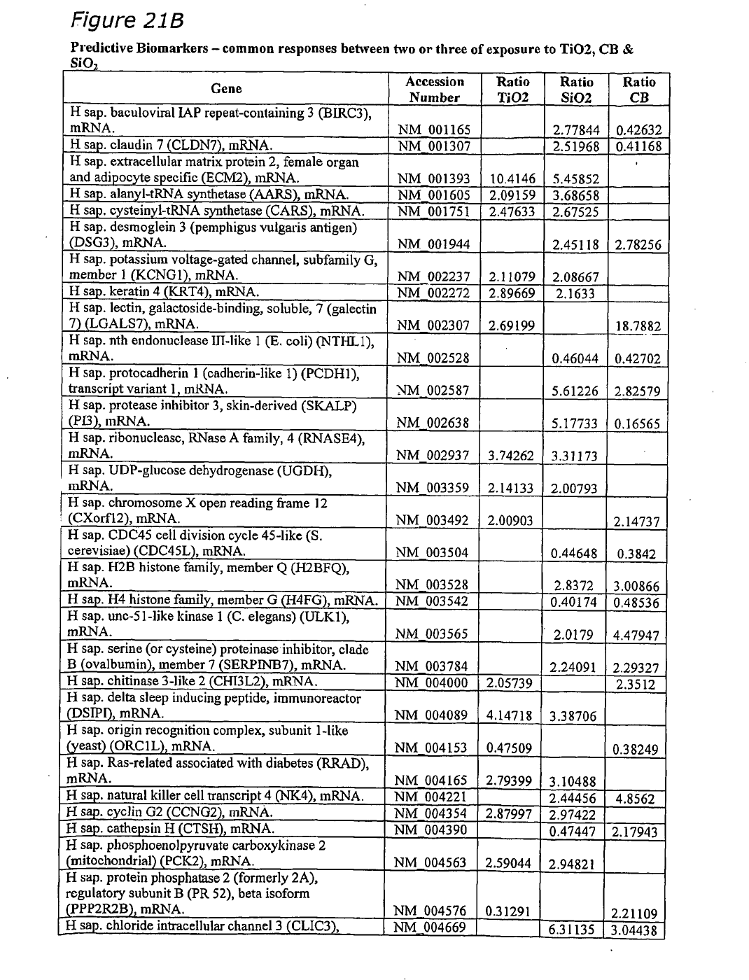

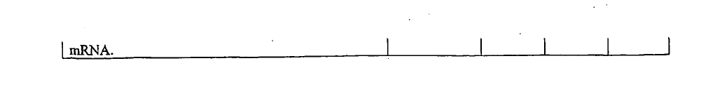

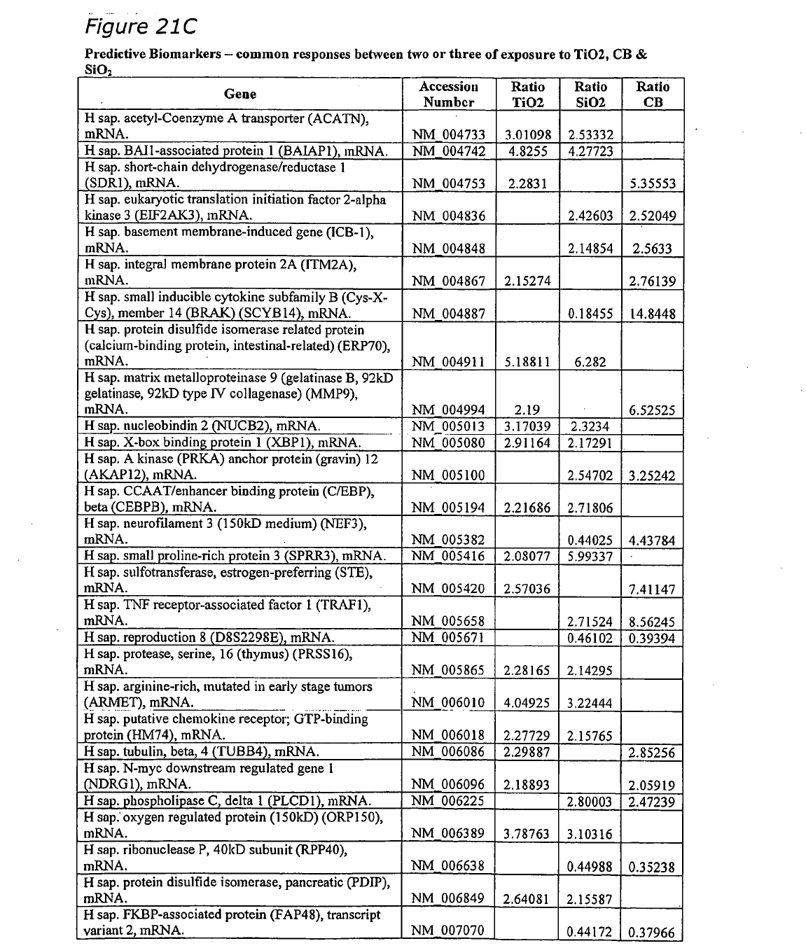

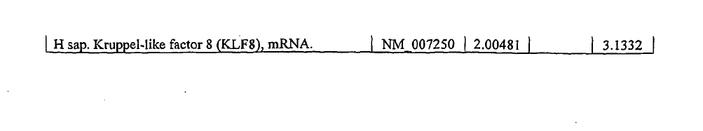

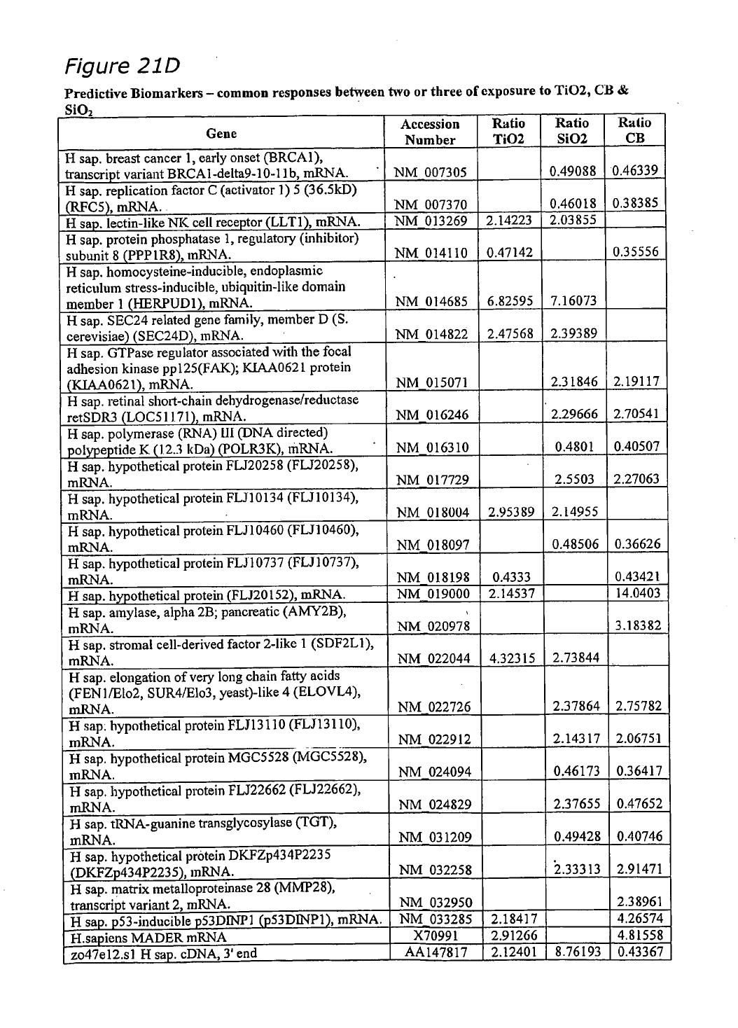

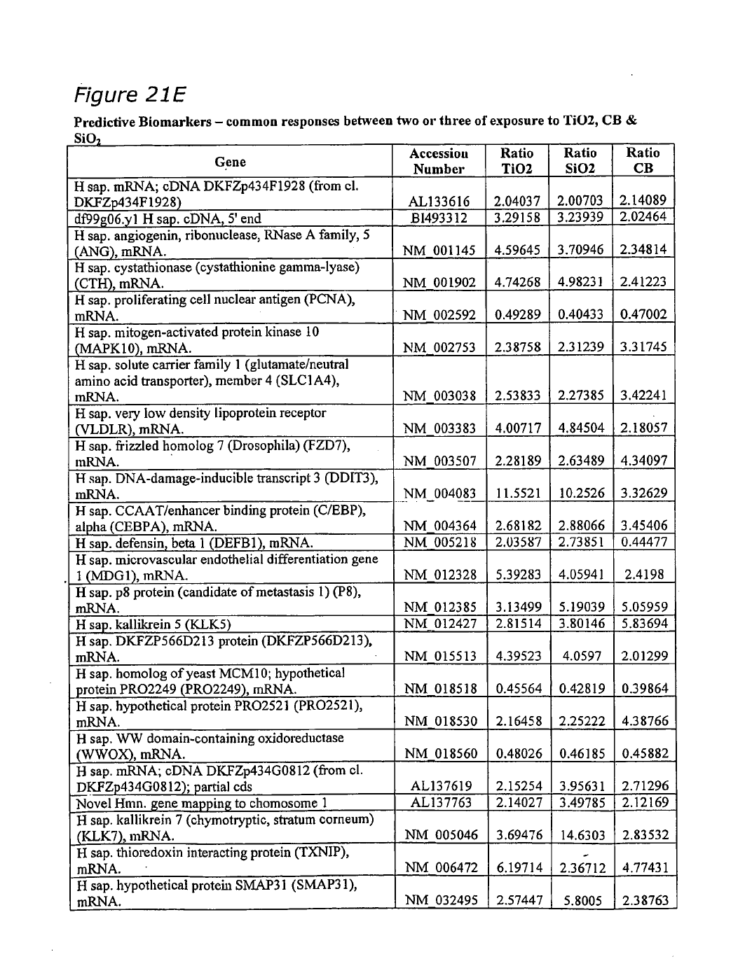

Predictive genes for exposure to TiO₂, CB and SiO₂: FIG. 21 depicts predictive biomarkers for nanomaterial exposure including genes significantly expressed up or down after exposure with two out of three of the three compounds, TiO₂, CB and SiO₂, or with all three based on the data presented in FIGS. 12A&B TiO₂,), 13A&B (CB), and 14A&B (SiO2).

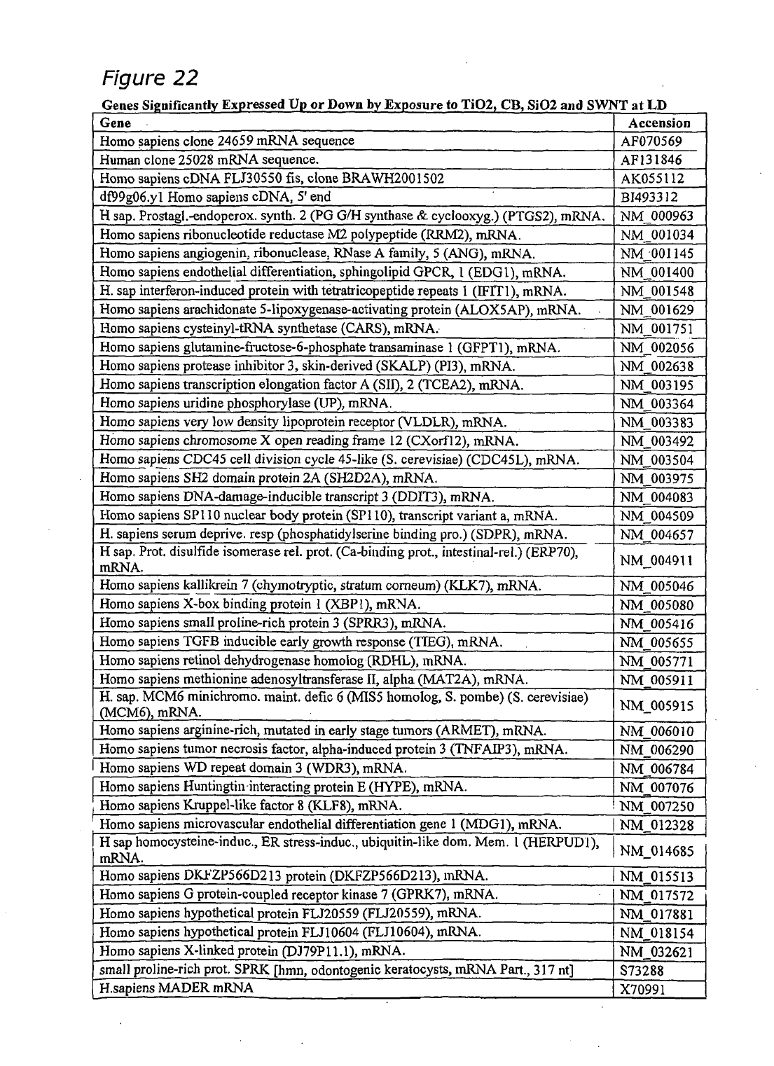

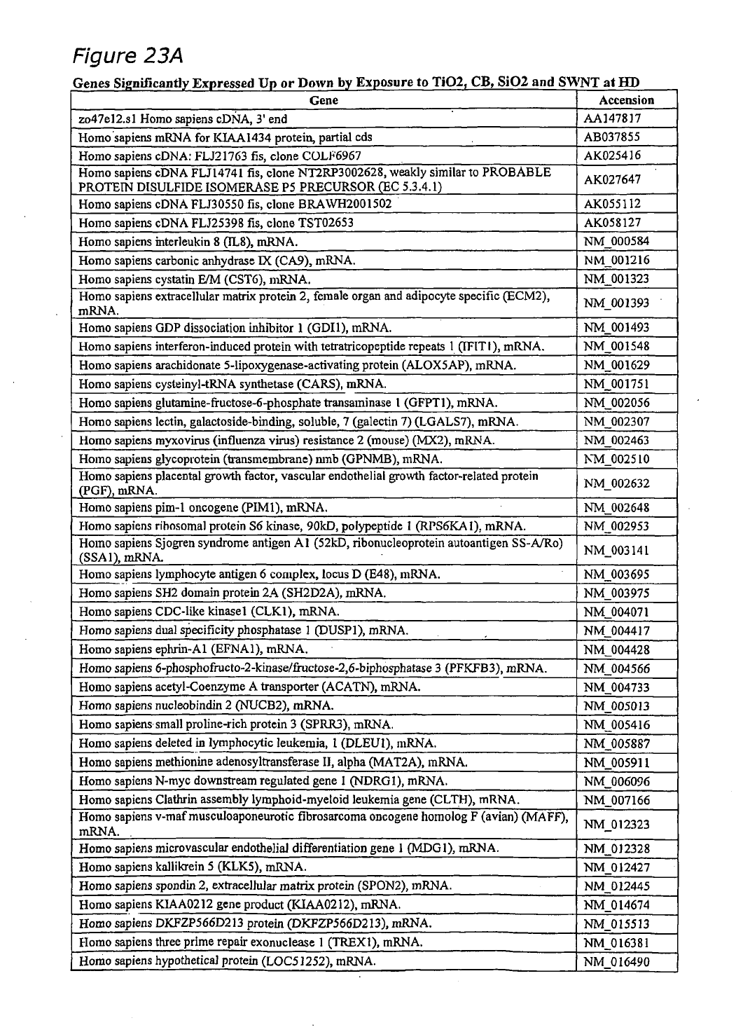

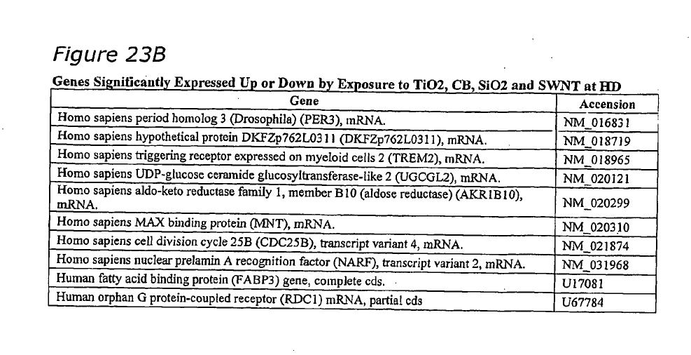

Predictive genes for exposure to TiO₂, CB, SiO₂ and SWNT: FIG. 22 is a table of genes significantly expressed across carbonyl iron, carbon black, silica and single-walled nanotubes at low dose (from the time coure studies). FIG. 23 A-B is a table of genes significantly expressed across carbonyl iron, carbon black, silica and single-walled nanotubes at high dose (from the time coure studies).

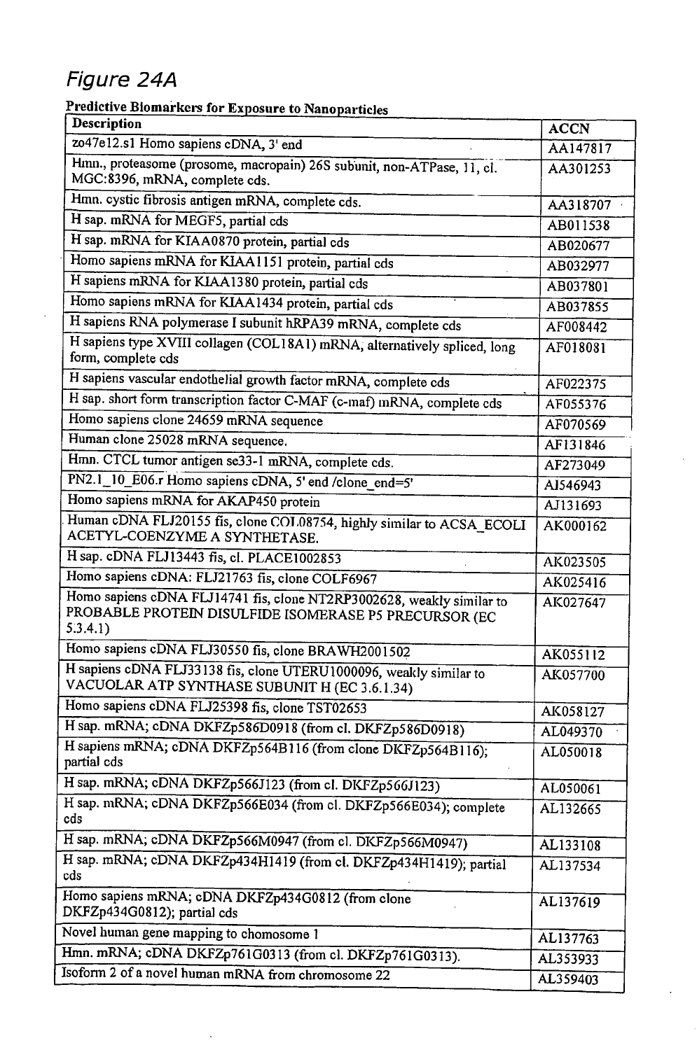

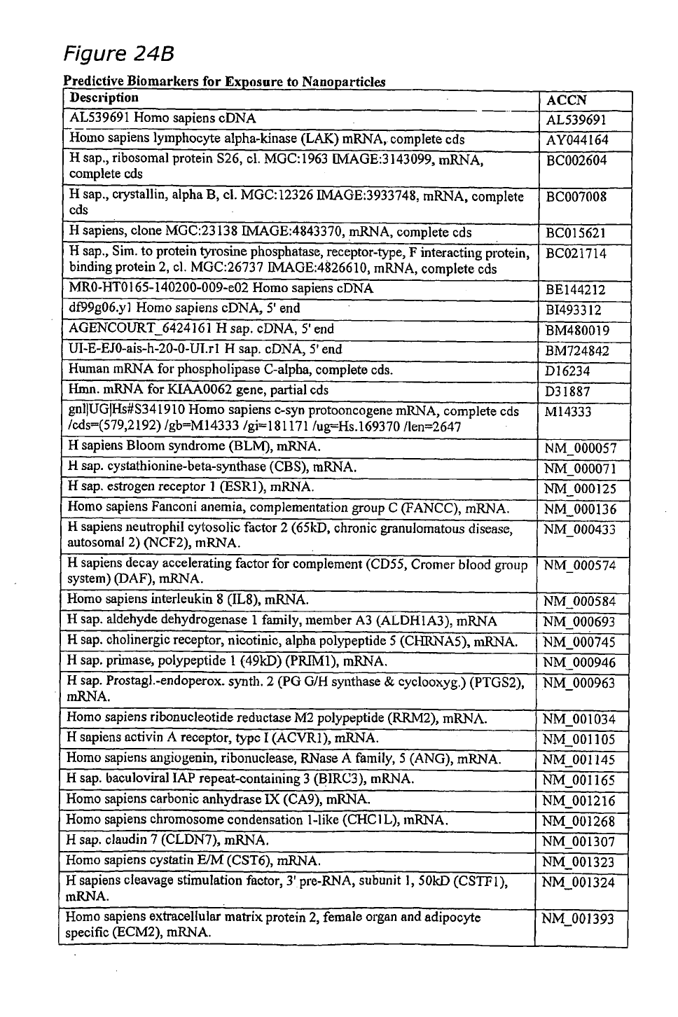

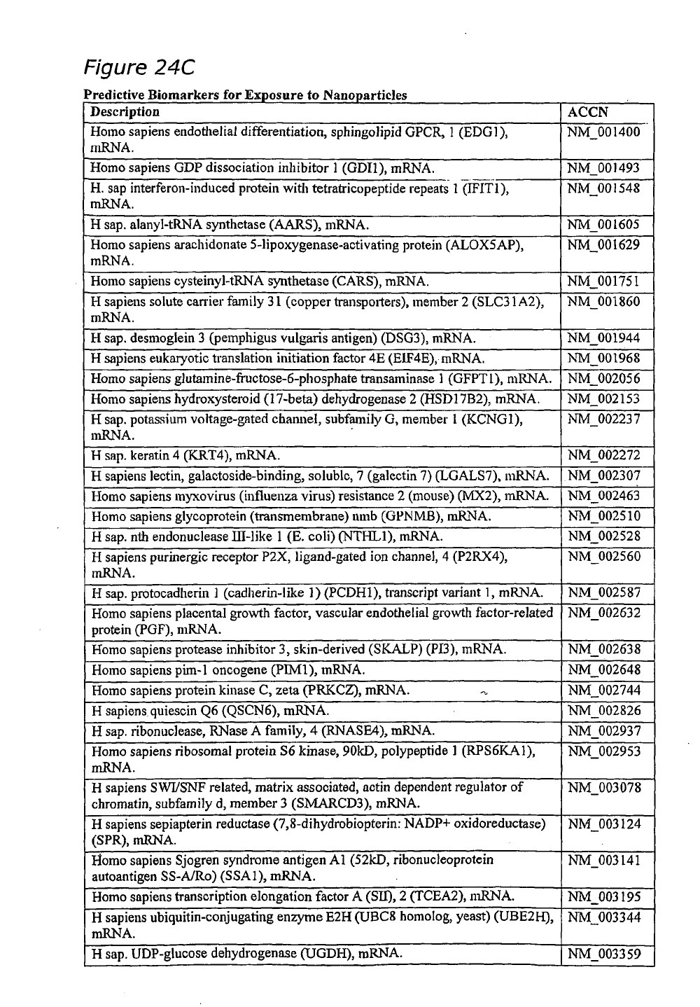

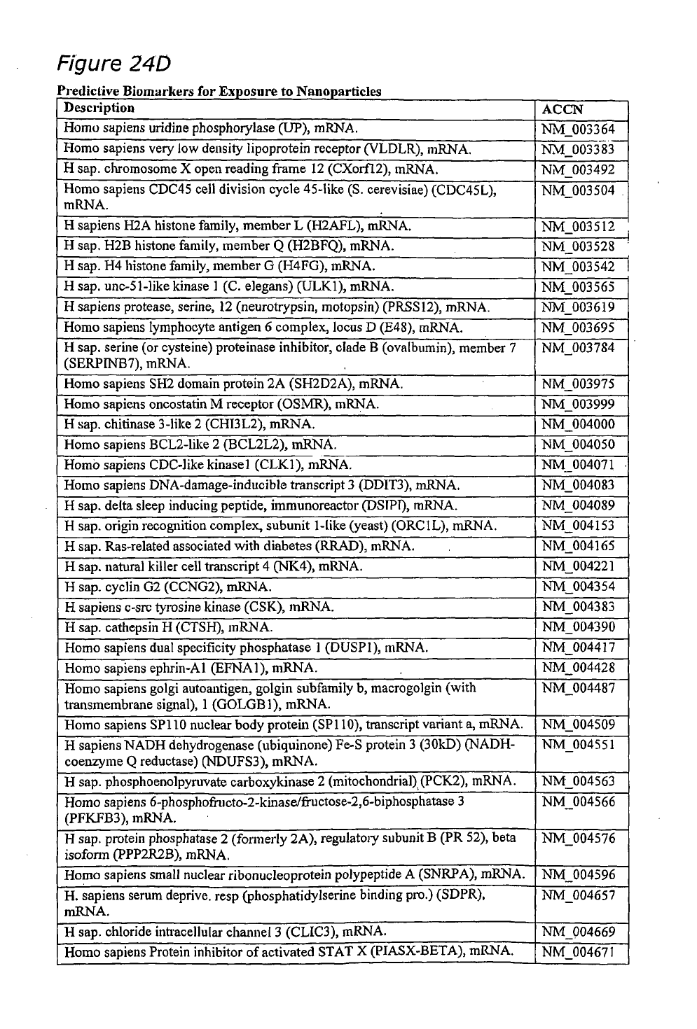

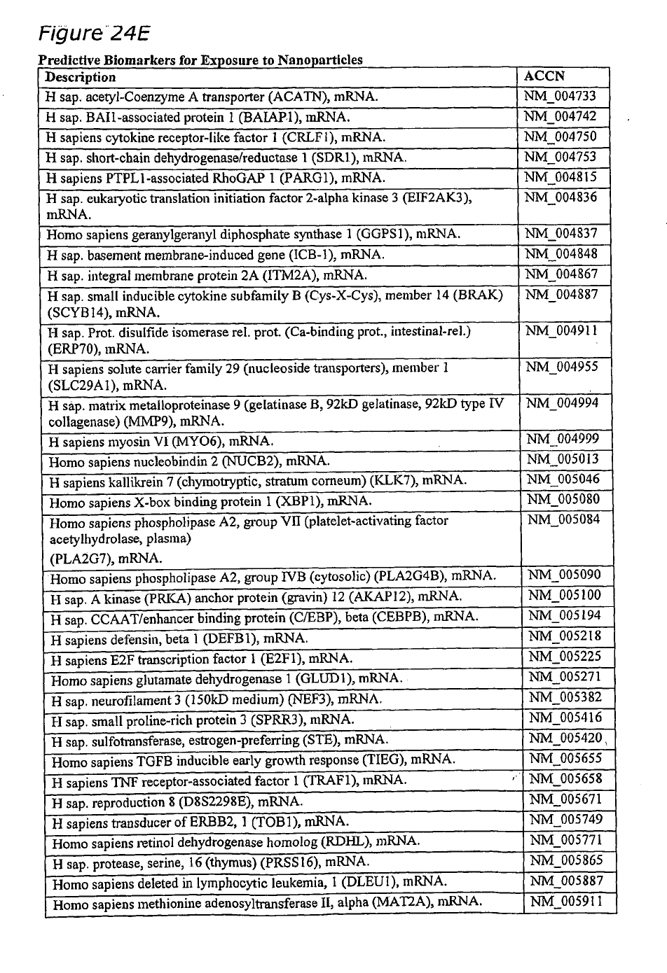

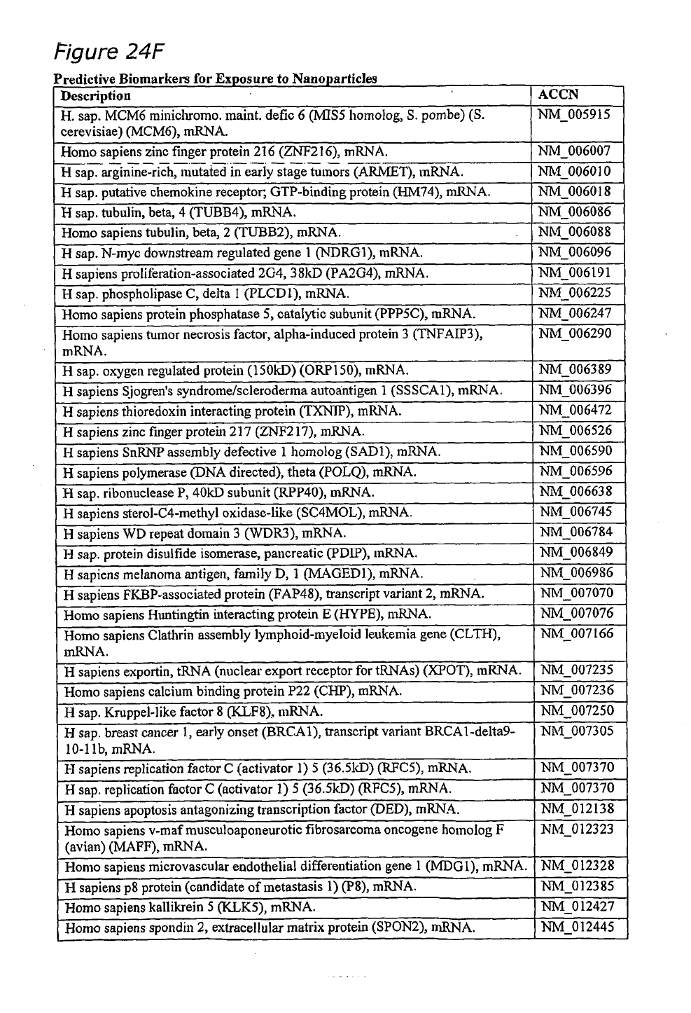

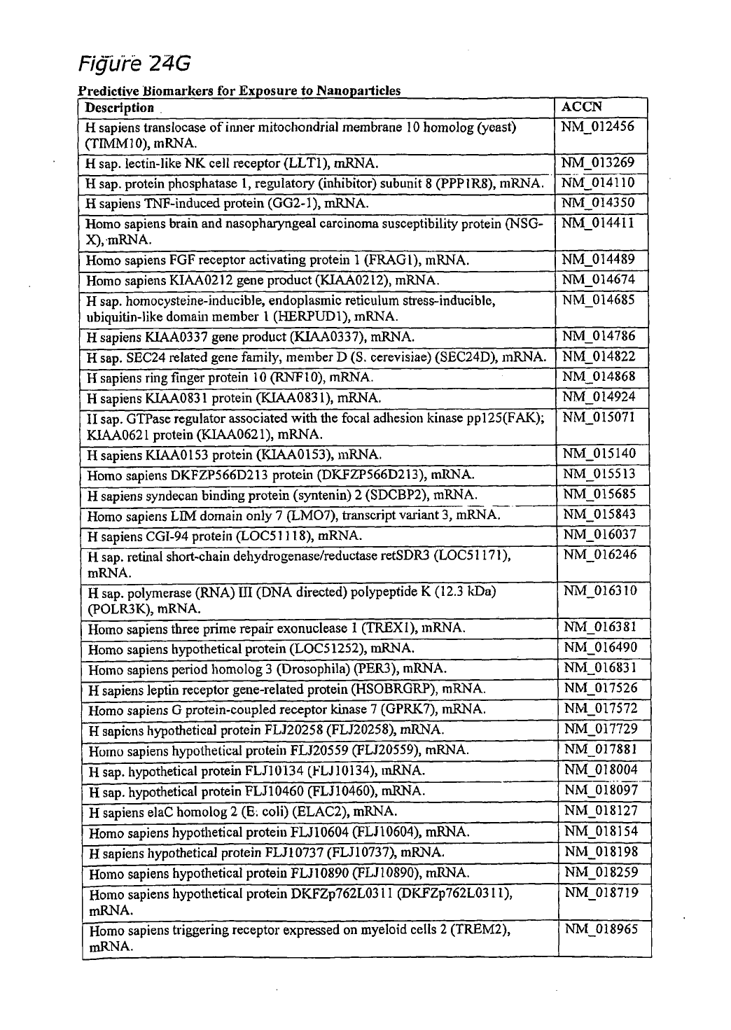

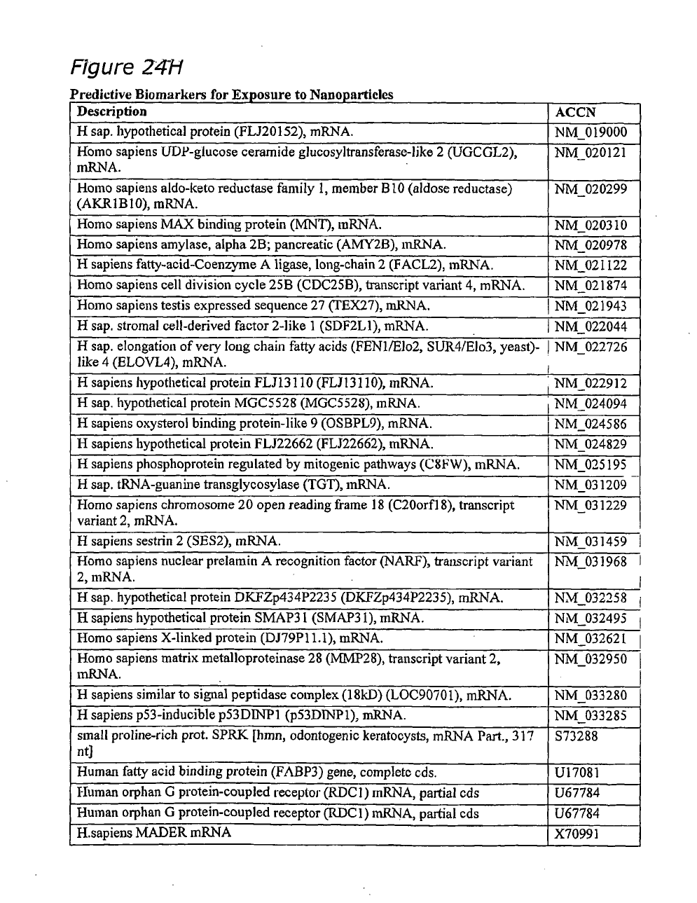

Predictive biomarkers for nanomaterial exposure: FIG. 24 is cumulative of genes identified in FIG. 21; genes listed in all LDA and QDA tables depicted in FIGS. 15 and 16, and genes common to all 4 compounds from time course series at both low (FIG. 22) and high dose (FIG. 23).

### EXAMPLE IV

### Testing of Exposure Unknowns

The biomarkers identified in the present studies can be used to identify exposure to nanoparticles in human and animal biology including, for example, in worker health exposure, consumer exposure to nanomaterials released over time or by damage to composite materials that include nanomaterials in their construction, and for detection in medical indications, including both toxicity and efficacy where the nanomaterial is used for drug delivery or as a pharmaceutical.

In one embodiment, cellular samples are obtained from the human or animal with possible exposure. Cellular lysates are produced and the samples are analysed for up or down regulation, or significantly changed expression of the genes identified herein. For example, epithelial cell derived samples may be obtained, for example by skin scrapings, bladder epithelia, needle biopsy, sputum samples, buccal scrapings, bronchilar lavage, etc. and processed for detection of the biomarkers disclosed herein.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A method for detecting exposure of a cell to a nanomaterial comprising:
a) generating a cDNA or cRNA population from a cell that has been in contact with, or is suspected of having been in contact with, a nanomaterial;
b) contacting the cDNA or cRNA under hybridization conditions with a microarray comprising a plurality of polynucleotide sequences that each represent genes or gene specific portions of genes, said microarray including one or more biomarker genes or gene specific portion of the biomarker genes that are up or down regulated by exposure to the nanomaterial; and
c) determining a relative degree of hybridization with the polynucleotide sequences comprising the microarray, as compared with a control sample;
wherein an increase or decrease relative degree of hybridization with the biomarker gene polynucleotide sequence indicates contact of the cell with the nanomaterial.

2. The method of claim 1, wherein the nanomaterial is selected from the group consisting of FC, SiO₂, CB, TiO₂, and CNT.

3. The method of claim 1 and 2, wherein the microarray includes polynucleotide sequences that each represent genes or gene specific portions of biomarker genes or gene families selected from the group set out on FIGS. 9A-C, and combinations thereof.

4. The method of claim 1, wherein the microarray includes polynucleotide sequences that each represent genes or gene specific portions of biomarker genes Kallikrein 5, Nice-1, and combinations thereof.

5. The method of claim 1, wherein the microarray includes polynucleotide sequences that each represent genes or gene specific portions of biomarker genes selected from the group consisting of: DNA-damage-inducible transcript 3 (DDIT3); serum/glucocorticoid regulated kinase (SGK); N-myc downstream regulated gene 1 (NDRG1); AXIN1 up-regulated (AXUD1); and combinations thereof.

6. The method of claim 3, wherein the biomarker genes are selected from the group consisting of: Kallikrein 5; Nice-1; Cystic fibrosis antigen Clone 24421; Hypothetical protein LOC221810; (LGALS7); S100 calcium binding protein A8 (S100A8); Uridine phosphorylase (UP); Bone morphogenetic protein receptor type IA (BMPR1A); Neurexin 2 (NRXN2); Rh type C glycoprotein (RHCG); Stromal cell-derived factor 2-like 1 (SDF2L1); Hypothetical protein SMAP31 (SMAP31); DNA-damage-inducible transcript 3 (DDIT3); serum/glucocorticoid regulated kinase (SGK); N-myc downstream regulated gene 1 (NDRG1); AXIN1 up-regulated (AXUD1); and combinations thereof.

7. The method of claim 1, wherein microarray includes a polynucleotide sequence that represents a biomarker gene or gene specific portion of the biomarker gene encoding Kallikrein 5 and one or more of the biomarker genes selected from the group consisting of: Nice-1; Cystic fibrosis antigen Clone 24421; Hypothetical protein LOC221810; (LGALS7); S100 calcium binding protein A8 (S100A8); Uridine phosphorylase (UP); Bone morphogenetic protein receptor type IA (BMPR1A); Neurexin 2 (NRXN2); Rh type C glycoprotein (RHCG); Stromal cell-derived factor 2-like 1 (SDF2L1); Hypothetical protein SMAP31 (SMAP31); DNA-damage-inducible transcript 3 (DDIT3); serum/glucocorticoid regulated kinase (SGK); N-myc downstream regulated gene 1 (NDRG1); AXIN1 up-regulated (AXUD1); and combinations thereof.

8. The method of claim 1, wherein microarray includes a polynucleotide sequence that represents a biomarker gene or gene specific portion of the biomarker gene encoding Nice-1 and one or more of the biomarker genes selected from the group consisting of: Kallikrein 5; Cystic fibrosis antigen Clone 24421; Hypothetical protein LOC221810; (LGALS7); S100 calcium binding protein A8 (S100A8); Uridine phosphorylase (UP); Bone morphogenetic protein receptor type IA (BMPR1A); Neurexin 2 (NRXN2); Rh type C glycoprotein (RHCG); Stromal cell-derived factor 2-like 1 (SDF2L1); Hypothetical protein SMAP31 (SMAP31); DNA-damage-inducible transcript 3 (DDIT3); serum/glucocorticoid regulated kinase (SGK); N-myc downstream regulated gene 1 (NDRG1); AXIN1 up-regulated (AXUD1); and combinations thereof.

9. The method of claim 1, wherein the microarray includes a polynucleotide sequence that represents a biomarker gene or gene specific portion of the biomarker gene or gene family encoding Nice-1 and one or more additional genes set out on FIGS. 9A-C, and combinations thereof.

10. The method of claim 1, wherein the microarray includes a polynucleotide sequence that represents a biomarker gene or gene specific portion of the biomarker gene or gene family encoding Kallikrein 5 and one or more additional genes set out on FIGS. 9A-C, and combinations thereof.

11. A method for detecting a toxicogenomic change in gene expression in cells exposed to a nanomaterial comprising:
a) generating a control cDNA or cRNA population from a population of control cells;
b) contacting a test cell population with a composition comprising a nanomaterial;
c) generating a test cDNA or cRNA population from the test cells after contact with the composition comprising the nanomaterial;
d) contacting the control and test cDNA or cRNA populations under hybridization conditions with microarrays comprising a plurality of polynucleotide sequences that each represent genes or gene specific portions of genes, said microarray including a nanomaterial biomarker set; and
e) determining a relative degree of microarray hybridization between with the control and test cDNA or cRNA;
wherein an increase or decrease relative degree of hybridization with one or more of the nanoparticle biomarker set between the control and test cDNA or cRNA indicates toxicogenomic change in gene expression in cells exposed one or more components of the composition comprising the nanomaterial.

12. A visual method for identification of nanoparticle exposure by cells, comprising comparing GEM profiles from exposed or putatively exposed cells with GEM profiles from control cells by three dimensional display of principal component analysis data.

13. The method of claim 11 wherein the biomarker set includes polynucleotide sequences representing genes or gene specific portions of genes identified on any one of FIGS. 9A-9C, FIG. 21, FIG. 22, FIG. 23 and FIG. 24.

14. The method of claim 11 wherein the biomarker set includes polynucleotide sequences representing genes or gene specific portions of a plurality of genes selected from the identified on any one of FIGS. 9A-9C and FIG. 24.

15. A biomarker set for identifying exposure of a cell to a nanomaterial wherein the biomarker set identifies up or down regulation of a plurality of the genes selected from the genes set out on any one of FIGS. 9A-C, 21, 22, 23 and 24.

16. A biomarker set for identifying nanoparticle exposure type on the basis of relative toxicity by up or down regulation of a plurality of genes selected from the genes set out on any one of FIGS. 15 and 16.

17. The biomarker set of claim 16, wherein the set comprises one or more of genes selected from the group consisting of: Homo sapiens cDNA FLJ10941 fis, clone OVARC1001243 (ACCN AK001803); Homo sapiens neurofibromin 1 (neurofibromatosis, von Recklinghausen disease, Watson disease) (NF1), mRNA (ACCN NM_{—}000267), Homo sapiens CDC-like kinasel (CLK1), mRNA (ACCN NM_{—}004071); Homo sapiens mRNA; cDNA DKFZp56402423 (from clone DKFZp56402423) (ACCN AL390214); Homo sapiens mRNA for KIAA0624 protein, partial cds (AB014524); and Homo sapiens cDNA: FLJ22917 fis, clone KAT06430 (AK026570).
